(19) United States
(12) Patent Application Publication
(10) Pub. No.: US 2024/0165259 A1
(43) Pub. Date: May 23, 2024

(54) BIOORTHOGONAL REACTION SUITABLE FOR CLICK/UNCLICK APPLICATIONS

(71) Applicant: DANA-FARBER CANCER INSTITUTE, INC., Boston, MA (US)

(72) Inventors: Justin Kim, Jamaica Plain, MA (US); Dahye Kang, Brookline, MA (US)

(73) Assignee: DANA-FARBER CANCER INSTITUTE, INC., Boston, MA (US)

(21) Appl. No.: 18/285,413

(22) PCT Filed: Apr. 4, 2022
(86) PCT No.: PCT/US2022/023325

§ 371 Date: Oct. 3, 2023

Related U.S. Application Data
(60) Provisional application No. 63/315,328, filed on Mar. 1, 2022.
(60) Provisional application No. 63/170,705, filed on Apr. 5, 2021.

(51) Int. Cl. A61K 47/68; A61K 47/62; A61K 49/00

(52) U.S. Cl. CPC A61K 47/6889; A61K 47/62; A61K 47/68031; A61K 47/6855; A61K 49/0056

(57) ABSTRACT

Disclosed are compounds and pharmaceutically acceptable salts and stereoisomers thereof that are suitable for cellular labeling or the treatment of cancer. Also disclosed are pharmaceutical compositions containing same, and methods of making and using the compounds.

### RELATED APPLICATIONS

This application claims the benefit of priority under 35 U.S.C. § 119(e) to U.S. Provisional Application No. 63/170,705, filed Apr. 5, 2021 and U.S. Provisional Application No. 63/315,328, filed Mar. 1, 2022, each of which are incorporated herein by reference in their entireties.

### GOVERNMENT SUPPORT

This invention was made with government support under grant number 1DP2 ES030448 awarded by The National Institutes of Health. The government has certain rights in the invention.

### SEQUENCE LISTING

The instant application contains a Sequence Listing which has been submitted electronically in ASCHII format and is hereby incorporated by reference in its entirety. Said ASCII copy, created on Apr. 4, 2022, is named 52095-726001WO_ST25.txt and is 6 KB bytes in size.

### BACKGROUND OF THE INVENTION

The advent of the copper-catalyzed azide-alkyne cycloaddition (CuAAC) reaction two decades ago (Rostovtsev et al., Angew. Chem., Int. Ed. 41(14):2596-2599 (2002)) has proven indispensable in fields ranging from materials science to chemical biology (Hein et al., Chem. Soc. Rev. 39:1302-1315 (2010); Neumann et al., Macromol. Rapid Commun. 41:1900359 (2020)). It is a reaction with widespread appeal for its rapid kinetics, ease of execution, and for the near universal adaptability of its reaction components. The relatively inert azide and terminal alkyne are two of the smallest functional groups available, and either component can be incorporated with ease into biological macromolecules, metabolites, and probes without imposing significant perturbations on the system under evaluation. The ability to incorporate these motifs into biological systems through unnatural amino acids using orthogonal tRNA systems and methionine auxotrophs (Kiick et al., Proc. Natl. Acad. Sci. U.S.A. 99(1):19-24 (2002); Prescher et al., Nat. Chem. Biol. 1(1):13-21 (2005); Plass et al., Angew. Chem., Int. Ed. 50(17):3878-3881 (2011)) through lipid and nucleotide modifications (Parker et al., Cell 180(4):605-632 (2020); Hang et al., Acc. Chem. Res. 44(9):699-708 (2011); Laguerre et al., Curr. Opin. Cell Biol. 53:97-104 (2018); Flores et al., Chem. Soc. Rev. 49:4602-4614 (2020)), or through metabolic engineering (Parker et al., Cell 180(4):605-632 (2020); Agard et al., Acc. Chem. Res. 42(6):788-797 (2009); Laughlin et al., Proc. Natl. Acad. Sci. U.S.A. 106(1):12-17 (2009)) have been transformational.

Bertozzi and co-workers have since expanded the application of the azide-alkyne cycloaddition to live cell and in vivo systems by employing strained cyclooctynes in lieu of copper catalysts (Baskin et al., Proc. Natl. Acad. Sci. USA 104(43):16793-16797 (2007)). Strain-promoted reactions are now a staple of the bioorthogonal compendium with notable examples featuring trans-cyclooctene (Blackman et al., J. Am. Chem. Soc. 130(41):13518-13519 (2008)), norbornene (Devaraj et al., Bioconjugate Chem. 19(12):2297-2299 (2008)), quadricyclane (Sletten et al., J. Am. Chem. Soc. 133(44):17570-17573 (2011)), and cyclopropane (Patterson et al., J. Am. Chem. Soc. 134(45):18638-18643 (2012); Row et al., J. Am. Chem. Soc. 139(21):7370-7375 (2017)) in inverse-electron demand Diels-Alder cycloadditions, dipolar cycloadditions, and phosphine ligations. Importantly, cyclooctyne has undergone substantial geometric (Dommerholt et al., Angew. Chem., Int. Ed. 49(49):9422-9425 (2010); Ning et al., Angew. Chem., Int. Ed. 47(12):2253-2255 (2008); Mbua et al., ChemBioChem 12(12):1912-1921 (2011); Jewett et al., J. Am. Chem. Soc. 132(11):3688-3690 (2010); de Almeida et al., Angew. Chem., Int. Ed. 51(10):2443-2447 (2012)) and electronic (Agard et al., ACS Chem. Biol. 1(10):644-648 (2006); Baskin etal., Proc. Natl. Acad. Sci. U.S.A. 104(43):16793-16797 (2007); Ni etal., Angew. Chem., Int. Ed. 54(4):1190-1194 (2015); Hu et al., J. Am. Chem. Soc. 142(44):18826-18835 (2020)) tuning in a bid to enhance reaction kinetics with azides (Agard et al., J. Am. Chem. Soc. 126(46):15046-15047 (2004)), tetrazines (Blackman et al., J. Am. Chem. Soc. 130(41):13518-13519 (2008); Yang et al., Angew. Chem., Int. Ed. 51(30):7476-7479 (2012)), sydnones (Tao et al., Chem. Commun. 54(40):5082-5085 (2018)), and diazo (Andersen et al., J. Am. Chem. Soc. 137(7):2412-2415 (2015)) compounds.

The growing compendium of bioorthogonal reactions has enabled the visualization, isolation, and manipulation of biomolecules in complex biological settings both in vitro and in vivo (Sletten et al., Angew. Chem., Int. Ed. 48(38):6974-6998 (2009); Parker et al., Cell 180(4):605-632 (2020); Takayama et al., Molecules 24(1):172 (2019)). These reactions have been instrumental in the study of primary and secondary metabolites such as sugars (Baskin et al., Proc. Natl. Acad. Sci. U.S.A. 104(43):16793-16797 (2007); Agard et al., Acc. Chem. Res. 42(6):788-797 (2009); Cioce et al., Curr. Opin. Chem. Biol. 60:66-78 (2021)) and lipids (Hang et al., Acc. Chem. Res. 44(9):699-708 (2011); Laguerre et al., Curr. Opin. Cell Biol. 53:97-104 (2018); Flores et al., Chem. Soc. Rev. 49:4602-4612 (2020)) as well as biomacromolecules (George et al., Chem. Commun.56:12307-12318 (2020)) whose modification by genetic means is neither practical nor possible. Consequently, demand continues to exist for additional bioorthogonal tools, particularly those that are more compact (Shih et al., J. Am. Chem. Soc. 137(32):10036-10039 (2015); Andersen et al., J. Am. Chem. Soc. 137(7):2412-2415 (2015)), rapid (Jewett et al., J. Am. Chem. Soc. 132(11):3688-3690 (2010); Darko et al., Chem. Sci. 5:3770-3776 (2014); Hu et al., J. Am. Chem. Soc. 142(44):18826-18835 (2020)), stable (Row et al., J. Am. Chem. Soc. 139(21):7370-7375 (2017); Tu et al., Angew. Chem., Int. Ed. 58(27):9043-9048 (2019)), regioselective (Grost et al., Org. Biomol. Chem. 13:3866-3870(2015)), functionally diverse (Volker et al., Angew. Chem., Int. Ed. 53(39):10536-10540 (2014); Li et al., Nat. Chem. Biol. 12(3):129-137 (2016); Versteegen et al., Angew. Chem., Int. Ed. 57(33):10494-10499 (2018); Carlson et al., J. Am. Chem. Soc. 140(10):3603-3612 (2018); Ji et al., Chem. Soc. Rev. 48:1077-1094 (2019)), and orthogonal both to biology and to themselves (Liang et al., J. Am. Chem. Soc. 134(43):17904-17907 (2012); Patterson et al., Curr. Opin. Chem. Biol. 28:141-149 (2015)).

The development of new reactions making simultaneous advances along not just one or two, but several of these axes, has been an enticing yet elusive aspiration (Row et al., Acc. Chem. Res. 51(5):1073-1081 (2018); Devaraj, N. K., ACS Cent. Sci. 4(8):952-959 (2018)).

### SUMMARY OF THE INVENTION

A first aspect of the present invention is directed to a compound represented by a structure of formula (I):

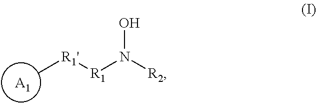

wherein R₁, R₁′, R₂, and A₁ are as defined herein, or a pharmaceutically acceptable salt or stereoisomer thereof.

Other aspects of the present invention are directed to compounds represented by formulas (II) and (III):

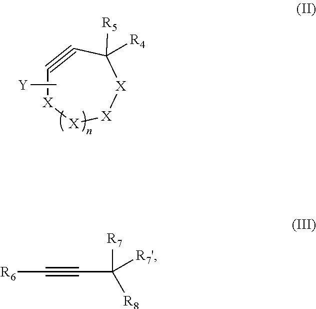

wherein R₄, R₅, R₆, R₇, R₇′, R₈, X, Y, and n are as defined herein, or a pharmaceutically acceptable salt or stereoisomer thereof.

Yet other aspects of the present invention are directed to enamine N-oxide compounds represented by formulas (IV) and (V):

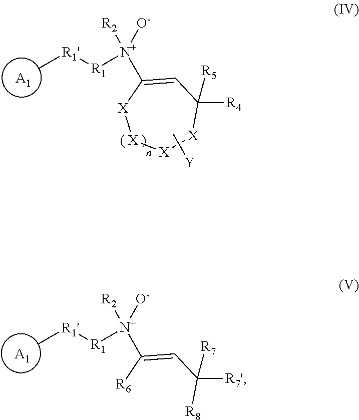

wherein R₁, R₁′, R₂, A₁, R₄, R₅, R₆, R₇, R₇′, R₈, X, Y, and n are as defined herein, or a pharmaceutically acceptable salt or stereoisomer thereof. Compounds of formulas (IV) and (V) each contain at least two active moieties.

Inventive compounds are particularly suited for clinical applications where delivering two active agents or moieties is advantageous. Therefore, in some embodiments, the compound of formula (IV) or (V) is an antibody-drug conjugate wherein one of the two active moieties is a binding moiety and the other active moiety is a therapeutic agent. In other embodiments, the compound of formula (IV) or (V) is a proteolysis-targeting chimera (also known as a PROTAC or degrader) that targets a given protein for selective degradation, wherein both of the active moieties are binding moieties. One of the binding moieties binds the target protein and the other binding moiety binds a cellular enzyme that catalyzes degradation of the target protein. Although both active moieties are binding moieties, the compound itself is therapeutic. In other embodiments, the compound of formula (IV) or (V) is a theranostic agent wherein one of the two active moieties is a diagnostic agent and the other active moiety is a therapeutic agent.

Further aspects of the present invention are directed to processes of preparing bifunctional enamine N-oxide compounds of formulas (IV) and (V) that carry two different active moieties. Processes for making compounds of formula (IV) entail reacting a compound of formula (I) with a compound of formula (II). Processes for making compounds of formula (V) entail reacting a compound of formula (I) with a compound of formula (III). The processes or synthetic methods by which compounds of formulas (IV) and (V) are made involve a bioorthogonal reaction between two reagents, namely compounds of formula (I) and compounds of formulas (II) and (III). More specifically, it is an uncatalyzed conjugative retro-Cope elimination reaction that enables the biorthogonal ligation of two active moieties.

Another aspect of the present invention is directed to a pharmaceutical composition that includes a therapeutically effective amount of a compound of formula (I-V) or a pharmaceutically acceptable salt or stereoisomer thereof, and a pharmaceutically acceptable carrier.

Further aspects of the present invention are directed to methods of diagnosing and treating diseases and disorders. In some embodiments, the disease is cancer. Other aspects of the present invention are directed to methods of protein labeling. In some embodiments, the methods are directed to labeling a cancer associated antigen.

The biorthogonal reaction is rapid and brings together (ligates) these two active moieties via a cleavable linker. The biorthogonal reaction may occur prior to administration to a subject or in vivo after administration of the individual reagents. That is, compounds (IV) and (V) may be administered to a subject. Alternatively, these compounds may be formed in vivo following administration of a compound of formula (I) and a compound of formula (II) or (III).

### BRIEF DESCRIPTION OF THE DRAWINGS

FIG. 1 is a schematic depicting a bioorthogonal retro-Cope elimination reaction between cyclooctynes and N,N-dialkylhydroxylamines.

FIG. 2A-FIG. 2D illustrate computational studies of the retro-Cope elimination reaction between cyclooctynes (COT) and N,N-dimethylhydroxylamine. Geometries were optimized at the M06-2X/6-31G(d,p) level of theory and single point energies were computed at the M06-2X/6-311G(2d,p) level of theory. FIG. 2A is a computational reaction model to evaluate the reactivity of cyclooctynes. FIG. 2B shows the calculated transition state structure and activation energy for cyclooctyne hydroamination. FIG. 2C shows that the additional ring strain of bicyclo[6.1.0]nonyne resulted in a lower activation barrier. FIG. 2D is a table of calculated free energies of activation (ΔG^{‡}) as well as distortion (ΔE^{‡}_{dist}.) and interaction energies (ΔE^{‡}ᵢₙₜ.) highlight the rapidity of the retro-Cope elimination reaction and the central role of hydroxylamine and alkyne distortion energies in lowering the activation barrier. R=p-NO₂Ph.

FIG. 3 shows the second-order rate constants for the hydroamination of cyclooctynes 2-10 by N,N-diethylhydroxylamine (1). Second-order kinetics studies were performed using equimolar concentrations of cyclooctyne and hydroxylamine at room temperature in CD₃CN. The rate constant for difluorocyclooctyne 10 was derived from competition experiments with carbamate 9.

FIG. 4A-FIG. 4E demonstrates protein labeling using the retro-Cope elimination reaction. FIG. 4A is a synthetic route for fluorophore-hydroxylamine conjugate 13. FIG. 4B shows that lysozyme was modified using N-Hydroxysuccinimide (NHS)-ester 14 to provide cyclooctyne-containing lysozyme 15. The modified protein lysozyme-COT 15 was labeled with fluorescent hydroxylamine 13. FIG. 4C is an in-gel fluorescence analysis of lysozyme-COT 15 (0.14 mg/mL) incubated with various concentrations of hydroxylamine 13 (10-200 μM) in phosphate-buffered saline (PBS) at room temperature for 2 hours. FIG. 4D is an in-gel fluorescence analysis of lysozyme-COT 15 (0.14 mg/mL) incubated with hydroxylamine 13 (200 μM) for 1-120 min in PBS at room temperature. FIG. 4E shows that complete conjugation was observed via intact mass spectrometry of lysozyme-fluorophore conjugate 16 obtained by incubation of lysozyme-COT 15 (0.58 mg/mL) and hydroxylamine 13 (200 μM) in PBS at room temperature for 6 hours.

FIG. 5A-FIG. 5D illustrates bioorthogonality. FIG. 5A shows the synthesis of enamine N-oxide 17. FIG. 5B is a bar graph showing the stability of hydroxylamine 13 and enamine N-oxide 17 that was studied in PBS at pH 7.4 in the presence of glutathione (5 mM), cell lysate (1 mg/mL), microsomes (0.2 mg/mL), or without additives. The protective effect of sodium ascorbate (5 mM) was additionally evaluated for hydroxylamine 13. FIG. 5C is an in-gel fluorescence analysis of the reaction between hydroxylamine 13 (200 μM) and lysozyme-COT 15 in the presence of cell lysate (2.5 mg/mL) for 2 hours showed exclusive labeling of lysozyme. FIG. 5D shows cross-reactivity between different sets of bioorthogonal components that were evaluated in CD₃CN at room temperature. R₁=CH₂NHBoc, R₂=C(O)NH(CH₂)₃NH₂, Ar=p-methylphenyl, R₃=C(O)NHCH(CH₃)₂, R₄=(CH₂)₂COOH.

FIG. 6A-FIG. 6H are reaction plots that were used to calculate second order rate constants between N,N-diethylhydroxylamines and cyclooctynes 2 (FIG. 6A), 3 (FIG. 6B), 4 (FIG. 6C), 5 (FIG. 6D), 6 (FIG. 6E), 7 (FIG. 6F), 8 (FIG. 6G), and 9 (FIG. 6H). Each panel shows n=3 separate experiments.

FIG. 7 shows the competition experiment performed between a 1:4 ratio of cyclooctyne carbamate 9 and difluorocyclooctyne 10 to determine the second order rate constant of the latter with N,N-diethylhydroxylamine.

FIG. 8 is an image of a full Coomassie stain (left) and in-gel fluorescence (right) image for concentration-dependent protein labeling experiments. Both images are from the same gel.

FIG. 9 is an image of a full Coomassie stain (left) and in-gel fluorescence (right) image for concentration-dependent protein labeling experiments. Both images are from the same gel.

FIG. 10A-FIG. 10C shows that mass spectrometry confirmed the bioorthogonal reaction between hydroxylamine 13 and lysozyme-COT 15. ESI mass spectra of unmodified lysozyme (FIG. 10A), lysozyme-cyclooctyne conjugate (FIG. 10B), and reaction mixture of hydroamination between hydroxylamine 13 and lysozyme-cyclooctyne conjugate 15 (FIG. 10C) (single adduct: expected 15073.3 Da, observed 15073.3 Da; double adduct: expected 15840.6 Da, observed 15841.7 Da).

FIG. 11A-FIG. 11C shows alkyne activation. FIG. 11A shows metal-catalyzed azide-alkyne cycloaddition. FIG. 11B shows train-promoted alkyne hydroamination. FIG. 11C shows hydroamination of a push-pull-activated linear alkyne.

FIG. 12A-FIG. 12B shows the effects of terminal and propargylic modification. FIG. 12A shows a reactivity screen using alkynes 8′-15′. NMR conversion with trifluorotoluene as internal standard. FIG. 12 B shows the synthesis of alkynes 9′-15′. R=OPMB. PMB=p-methoxybenzyl.

FIG. 13A-FIG. 13B shows reaction kinetics and stability of select alkynes and enamine N-oxides. FIG. 13A is a table of second-order rate constants of alkynes 11′-15′ in CD₃CN at room temperature. FIG. 13B is a graph showing the stability of alkynes 13′, 14′, 15′ and enamine N-oxide 20′ in 50% CD₃CN/PBS in the presence or absence of glutathione (GSH) or HEK293T cell lysate.

FIG. 14A-FIG. 14E shows in vitro and live cell labeling by bioorthogonal hydroamination. FIG. 14A shows that HaloTag protein was conjugated to chloroalkyne 21′ and modified by TAMRA-hydroxylamine 22′ then visualized by in-gel fluorescence or fluorescence microscopy. FIG. 14B depict structures of chloroalkyne 21′ and TAMRA-hydroxylamine 22′.

FIG. 14C is a time-dependent in-gel fluorescence analysis of hydroamination between alkyne 21′ and hydroxylamine 22′ (200 μM) for 1-60 min at room temperature. FIG. 14D is a concentration-dependent in-gel fluorescence analysis of hydroamination between alkyne 21′ (30 μM) and hydroxylamine 22′ (25-200 μM) upon incubation for 2 hours at room temperature. FIG. 14E is a series of images showing that cell surface HaloTag-GFP expressed on HEK293T cells was labeled with TAMRA by bioorthogonal hydroamination between alkyne 21′ and hydroxylamine 22′. Merge is a composite of Hoechst 33342, GFP, and TAMRA channels. Scale bar=50 μm. TAMRA=tetramethylrhodamine.

FIG. 15A-FIG. 15B depicts computational studies on the effects of alkyne halogenation.

FIG. 15A is a table of s-Characters (s-char) of alkyne sp-carbons that were analyzed, and activation free energies (ΔG^{‡}) were computed for the reaction of alkynes with hydroxylamine 24′. FIG. 15B is a graph showing the correlation of s-character and activation free energy.

FIG. 16A-FIG. 16E is a series of reaction plots that were used to calculate second order rate constants between alkynes (11′-15′) and N,N-diethylhydroxylamine. FIG. 16A is a graph for alkyne 11′ (2 mM) and hydroxylamine 2′ (20-40 mM). FIG. 16B is a graph for alkyne 12′ (2 mM) and hydroxylamine 2′ (18-37 mM). FIG. 16C is a graph for alkyne 13′ (10 mM) and hydroxylamine 2′ (10 mM). FIG. 16D is a graph for alkyne 15′ (10 mM) and hydroxylamine 2′ (10 mM). FIG. 16E is a graph for alkyne 14′ (10 mM) and hydroxylamine 2′ (10 mM). Each panel shows results for experiments performed in triplicate.

FIG. 17 is a series of ¹⁹F NMR spectra that shows compound 14′ (2 mM) is stable in 50% CD₃CN/PBS (pH 7.0) for 2 weeks.

FIG. 18 is a series of ¹⁹F NMR spectra that shows compound 14′ (500 μM) has a half-life of 14 hours in 50% CD₃CN/PBS (pH 7.0) in the presence of glutathione (2 mM) and is sufficiently stable for bioorthogonal transformations over 8 hours.

FIG. 19 is a series of ¹⁹F NMR spectra that shows compound 15′ (2 mM) is stable in 50% CD₃CN/PBS (pH 7.0) for 1 week.

FIG. 20 is a series of ¹⁹F NMR spectra that shows compound 15′ (500 μM) has a half-life of 43 hours in 50% CD₃CN/PBS (pH 7.0) in the presence of glutathione (2 mM) and is sufficiently stable for bioorthogonal transformations over 8 hours.

FIG. 21A-FIG. 21B is a full in-gel fluorescence image (FIG. 21A) and a Coomassie stain image (FIG. 21B) for time-dependent protein labeling experiments. Both images are from the same gel. The molecular weights for the ladder in the in-gel fluorescence image were identified and labeled using an image with increased contrast settings.

FIG. 22A-FIG. 22B is a full in-gel fluorescence image (FIG. 22A) and Coomassie stain image (FIG. 22B) for time-dependent protein labeling experiments. Both images are from the same gel. The molecular weights for the ladder in the in-gel fluorescence image were identified and labeled using an image with increased contrast settings.

FIG. 23A-FIG. 23C show that mass spectrometry confirmed the bioorthogonal reaction between hydroxylamine 22′ and alkyne S15′. ESI mass spectra of unmodified HaloTag protein (FIG. 23A), HaloTag-alkyne conjugate (expected 34981 Da, observed 34981 Da) (FIG. 23B), and reaction mixture of hydroamination between hydroxylamine 22′ and HaloTag-alkyne conjugate (expected 35527 Da, observed 35529 Da) (FIG. 23C).

FIG. 24 shows the s-Character of enamine N-oxide sp²-carbons (C₂).

FIG. 25A-FIG. 25D depict bioorthogonal transformations. FIG. 25A shows an associative bioorthogonal transformation. FIG. 25B shows a dissociative bioorthogonal transformation. FIG. 25C shows chemically reversible bioconjugation. FIG. 25D shows a rapid and complete sequential biorthogonal hydroamination and traceless release of biomolecules via enamine N-oxides.

FIG. 26A-FIG. 26B show the evaluation of the impact of hydroxylamine substitutents on the biorthogonal retro-Cope elimination reaction. FIG. 26A shows the synthetic route for accessing TAMRA-hydroxylamine conjugates 6″-9″. FIG. 26B shows a series of in-gel fluorescence images and Coomassie stain images for lysozyme-cyclooctyne conjugate 11″ (10 μM) which was incubated with TAMRA-hydroxylamine conjugates 6″_{conj}-10″_{conj} (200 μM) in PBS at room temperature for 1-72 h.

FIG. 27A-FIG. 27D illustrates the computational studies investigating the formation and degradation of enamine N-oxide structures. FIG. 27A shows a computational reaction model exploring the effect of steric hindrance on the hydroamination and Cope elimination reactions.

FIG. 27B shows the calculated Gibbs free energies and free energies of activation. Reaction coordinates for Path A and B are in blue and red, respectively. FIG. 27C shows the three-dimensional structures of 17″ and 18″ and Path A and B transition state structures 17″-TSa and 18″-TSb. FIG. 27D shows the reaction between cyclooctyne 22″ (2 mM) and hydroxylamine 4″ (2 mM) was monitored by A220 absorbance on LCMS.

FIG. 28A-FIG. 28E illustrates diboron-mediated enamine N-oxide reduction and payload release. FIG. 28A is a reaction scheme for enamine N-oxide-bearing lysozyme-TAMRA conjugates 6″_{conj}, 9″_{conj}, and 10″_{conj} treated with diboron reagents in PBS at room temperature to induce the release of the fluorophore. FIG. 28B is a series of in-gel fluorescence images and silver stain images for concentration-dependent cleavage of lysozyme-TAMRA conjugates 6″_{conj}, 9″_{conj}, and 10″_{conj}(480 nM) at room temperature over 1 h with B₂pin₂ (5-50 μM) was analyzed together with time-dependent cleavage over 5-60 min with 5 μM B₂pin₂ by in-gel fluorescence. Silver stain is provided as loading control. FIG. 28C is a series of graphs showing the quantification of the fluorescence in the bands from the time-dependent diboron-induced cleavage experiment. FIG. 28D shows complete conjugation and removal of TAMRA from lysozyme by mass spectrometry. Lys-COT 11″ (10 μM) featuring 0-3 modifications was combined with hydroxylamine 6″ (200 μM) in PBS at room temperature for 6 h. FIG. 28 E depicts the in-gel fluorescence images and silver stain images for structurally diverse diboron reagents 27″-31″ (5 or 50 μM) that were incubated with N-methyl lysozyme-TAMRA conjugate 6″_{conj}(240 nM) for 60 min at room temperature.

FIG. 29A-FIG. 29B shows the characterization of the diboron-mediated reductive cleavage of enamine N-oxides. FIG. 29A shows the progress of the reaction between 4 mM p-nitrophenol-derived enamine N-oxide 32″ and 10 mM B₂(OH)₄ in 10% DMSO-d₆/23% CD₃OD/67% d-PBS, pH 7.4 which was monitored by ¹H NMR spectroscopy over 24 h. FIG. 29B shows the progress of the reaction between p-nitrophenyl thioether 38″ and p-nitrophenylcarbamate 39″.

FIG. 30A-FIG. 30E shows the investigation of the reaction scope and kinetics of payload release for the diboron-mediated cleavage of enamine N-oxides. FIG. 30A shows the reaction scheme for the synthesis of lysozyme-fluorescein conjugate 41″ by hydroamination of Lys-COT 11″ with fluorescein hydroxylamine 40″ in PBS at room temperature. FIG. 30B shows the kinetics of diboron-mediated enamine N-oxide cleavage that was determined by fluorescence polarization under pseudo-first order conditions when lysozyme-fluorescein conjugate 41″ (500 nM) was treated with B₂pin₂ (25-200 μM) in PBS at room temperature. FIG. 30C is a graph that depicts the influence of buffer pH on cleavage rates. Lysozyme-fluorescein conjugate 41″ (500 nM) was reduced with B₂pin₂ (100 μM) in PBS, pH 4-10, and conversion was measured by fluorescence polarization. FIG. 30D is a graph that depicts the influence of buffer composition on cleavage rates. Lysozyme-fluorescein conjugate 41″ (500 nM) was reduced with B₂pin₂ (50 μM) in several buffers, and conversion was measured by fluorescence polarization. FIG. 30E is a series of graphs that shows the influence of leaving group composition on cleavage rates.

FIG. 31A-FIG. 31D shows the synthesis and cellular evaluation of chemically cleavable enamine N-oxide-linked antibody-drug conjugates. FIG. 31A shows the synthesis of ADCs 61″ and 62″. FIG. 31B is a graph of a cell viability assay of trastuzumab-derived ADC 61″ in the presence or absence of 50 μM B₂pin₂ on SK-BR-3 HER2⁺ breast cancer cells. FIG. 31C is a graph of a cell viability assay of trastuzumab-derived ADC 61″ in the presence or absence of 50 μM B₂pin₂ on MDA-MB-231 HER2⁻ breast cancer cells. FIG. 31D is a graph of a cell viability assay of IgG isotype control-derived ADC 62″ in the presence or absence of 50 μM B₂pin₂ on SK-BR-3 HER2⁺ breast cancer cells. ND=not determined. Error bars represent standard deviation (n=3).

FIG. 32A-FIG. 32B shows that protein modification using enamine N-oxide chemistry is traceless and reversible. FIG. 32A is a schematic illustration of sequential conjugation of removal of small molecules on lysozyme. FIG. 32B depicts that clean and complete click and release was observed by intact mass spectrometry.

FIG. 33 shows the reductive release of p-nitrothiophenol (S3″) from enamine N-oxide 38″ by B₂(OH)₄ at room temperature. ¹H NMR spectrum of the reaction in the presence of caffeine internal standard (A) before diboron addition, (B) 4 min after addition, and (C) 30 min after addition.

FIG. 34 shows the reductive release of p-nitroaniline (24″) from enamine N-oxide 39″ by B₂(OH)₄ at room temperature. ¹H NMR spectrum of the reaction in the presence of caffeine internal standard (A) before diboron addition, (B) 5 min after addition, and (C) 30 min after addition.

FIG. 35 is a full Coomassie stain (top) and in-gel fluorescence (bottom) image for time-dependent protein labeling experiments for compounds 6″ and 10″. Both images are from the same gel.

FIG. 36 is a full Coomassie stain (top) and in-gel fluorescence (bottom) images for time-dependent protein labeling experiments for compound 7″ and 8″. Both images are from the same gel.

FIG. 37 is a full Coomassie stain (left) and in-gel fluorescence (right) images for time-dependent protein labeling experiments for compound 9″. Both images are from the same gel.

FIG. 38 is a full in-gel fluorescence (left) and Oriole stain (right) images for the stability assays of enamine N-oxide protein conjugates 6″_{conj}, 9″_{conj}, and 10″_{conj} in PBS (pH 7.4). Both images are from the same gel.

FIG. 39 is a full in-gel fluorescence (left) and Oriole stain (right) images for the stability assays of enamine N-oxide protein conjugates 6″_{conj}, 9″_{conj}, and 10″_{conj} in RPMI. Both images are from the same gel.

FIG. 40 is a full in-gel fluorescence (left) and Oriole stain (right) images for the stability assays of enamine N-oxide protein conjugates 6″_{conj}, 9″_{conj}, and 10″_{conj} in RPMI supplemented with 10% fetal bovine serum. Both images are from the same gel.

FIG. 41A-FIG. 41B shows the evaluation of diboron reagents for the cleavage of enamine N-oxide-linked lysozyme-fluorophore conjugate 6″_{conj}. FIG. 41A depicts the structures of diboron substrates 27″-31″. FIG. 41B is an in-gel fluorescence and silver stain image of the diboron reagents shown in FIG. 41A. Both images are from the same gel.

FIG. 42A-FIG. 42C is a series of full in-gel fluorescence and quantification of each band for enamine N-oxide-linked lysozyme-fluorophore conjugate 6″_{conj}, 9″_{conj}, and 10″_{conj}. FIG. 42A is a full in-gel fluorescence and quantification of 6″_{conj}. FIG. 42B is a full in-gel fluorescence and quantification of 10″_{conj}. FIG. 42C is a full in-gel fluorescence and quantification of 9″_{conj}.

FIG. 43 shows the monitoring of a reaction between cyclooctyne 22″ (2 mM) and hydroxylamine 3″ (2 mM) by A220 absorbance on LCMS.

FIG. 44A-FIG. 44B shows the confirmation of the bioorthogonal click and release reaction of structurally diverse enamine N-oxides by mass spectrometry. FIG. 44A is a series of ESI mass spectra of unmodified lysozyme, lysozyme-cyclooctyne conjugate 11″, the hydroamination ligation reaction between hydroxylamine 6″ and lysozyme-COT 11″ (single adduct: expected 15073.1 Da, observed 15074.0 Da; double adduct: expected 15840.5 Da, observed 15842.7 Da), and the diboron-induced cleavage reaction of enamine N-oxide-linked conjugate 6″_{conj}(single adduct: expected 14376.8 Da, observed 14376.9 Da; double adduct: expected 14447.8 Da, observed 14447.3 Da). FIG. 44B is a series of ESI mass spectra of the hydroamination ligation reaction between hydroxylamine 9″ and lysozyme-COT 11″ (single adduct: expected 15041.1 Da, observed 15042.1 Da; double adduct: expected 15776.4 Da, observed 15778.5 Da), the diboron-induced cleavage reaction of enamine N-oxide-linked conjugate 9″_{conj}(single adduct: expected 14376.8 Da, observed 14377.7 Da; double adduct: expected 14447.8 Da, observed 14447.3 Da), the hydroamination ligation reaction between hydroxylamine 10″ and lysozyme-COT 11″ (single adduct: expected 15149.1 Da, observed 15149.7 Da; double adduct: expected 15992.5 Da, observed 15994.0 Da), and the diboron-induced cleavage reaction of enamine N-oxide-linked conjugate 10″_{conj}(single adduct: expected 14376.8 Da, observed 14376.1 Da; double adduct: expected 14447.8 Da, observed 14447.3 Da).

FIG. 45 is a series of gels showing that when enamine N-oxide bearing lysozyme-fluorescein conjugates 41″ and 48″-52″ were treated with B₂pin₂ in PBS at room temperature, it induced the release of the fluorophore.

FIG. 46 is a graph showing the influence of the structure of diboron reagent on cleavage rates.

FIG. 47 shows the structures of antibody-nitroaniline conjugates S22″-S24″.

FIG. 48A-FIG. 48B is a series of diboron reagent dose response curves from cell viability assays in SK-BR-3 cells. Cells were treated with B₂pin₂ (FIG. 48A) or B₂(OH)₄ (FIG. 48B) for 72 h. Error bars represent mean±SEM of data from biological replicates (n=3).

FIG. 49A-FIG. 49B is a series of diboron reagent dose response curves from cell viability assays in MDA-MB-231 cells. Cells were treated with B₂pin₂ (FIG. 49A) or B₂(OH)₄ (FIG. 49B) for 96 h. Error bars represent mean±SEM of data from biological replicates (n=3).

FIG. 50 is an in-gel fluorescence of enamine N-oxide bearing lysozyme-fluorescein conjugate 65″ that was treated with B₂pin₂ in PBS at room temperature to induce the release of the fluorophore.

FIG. 51 is a series of reaction coordinates for the bioorthogonal hydroamination reaction between cyclooctyne 12″ and hydroxylamines 14″ and 15″.

FIG. 52 is a graph showing the kinetic assay for enamine N-oxide-linked lysozyme-fluorescein conjugate 41″.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about”.

The transitional term “comprising,” which is synonymous with “including,” “containing,” or “characterized by,” is inclusive or open-ended and does not exclude additional, unrecited elements or method steps. When used in the context of the number of heteroatoms in a heterocyclic structure, it means that the heterocyclic group that that minimum number of heteroatoms. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “biorthogonal reaction” refers to any chemical reaction that can occur inside of a living system without interfering with native biochemical processes.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group, or a methyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O— alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C5-C6. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

The term “carbocyclic” also embraces “aryl” groups. As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene.

In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

The term “heterocyclic” also embraces “heteroaryl” groups. In some embodiments, a heterocyclyl refers to a heteroaryl ring system, such as a 5 to 14 membered heteroaryl ring system. As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may include alkyl, substituted alkyl (e.g., C₁-C₆, C₁-C₅, C₁-C₄, C₁-C₃, C₁-C₂, C1), alkoxy (e.g., C₁—C, C₁-C₅, C₁-C₄, C₁-C₃, C₁-C₂, C₁), substituted alkoxy (e.g., C₁—C, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁—C), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

As used herein, the term “π-electron withdrawing group” refers to functional group containing π-electrons which has a formal +ve or δ +ve charge, such as a carbonyl or nitro group, that attracts electron density.

As used herein, the term “inductive electron withdrawing group” refers to an atom or functional group containing an electronegative atom that attracts more electron density from the atoms to which they are attached, such as a fluoro or alkoxy group.

As used herein, the term “small molecule” refers to a molecule, whether naturally-occurring or artificially created (e.g., via chemical synthesis) that has a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.).

As used herein, the term active moiety refers to distinct, definable portion or unit of an inventive compound that performs some function or activity or that is reactive with other molecules. Representative types of active moieties include binding moieties, therapeutic moieties diagnostic moieties, and immobilizing moieties.

As used herein, the term “immobilizing moiety” refers to a portion of an inventive compound that is insoluble to which the rest of the inventive compound is bound to (e.g., through a covalent bond or encapsulation in a polymer matrix.

As used herein, the term “binding moiety” refers to a portion of an inventive compound that targets it to an appropriate site of action, e.g., a cancer associated antigen on a solid tumor cell.

As used herein, “therapeutic moiety” refers to a portion of an inventive compound that provides a therapeutic effect with respect to a disease or disorder when it reaches its intended site of action.

As used herein, the terms “diagnostic moiety” and “detectable moiety” are used interchangeably and refer to a portion of an inventive compound that provides a diagnostic effect in connection with a disease or disorder and permits visualization of cells or tissues in which inventive compounds accumulate.

In one aspect, compounds of the invention are represented by formula (I):

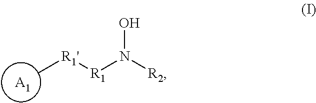

or a pharmaceutically acceptable salt or stereoisomer thereof,
wherein:
   R₁′ is a linking group;
   R₁ is absent, or
   R₁ and R₂, together with the nitrogen atom to which they are attached, form a heterocyclyl;
   R₂ is optionally substituted (C₁-C₈) alkyl, —C(O)R″, —C(O)OR″, —C(O)NR″R″, —S(O)R″, —S(O)₂R″, (C₃-C₁₀) carbocyclyl, 4- or 7-membered heterocyclyl, or a substituted polyethylene glycol chain, wherein each R″ is independently hydrogen, (C₁-C₆) alkyl, (C₃-C₁₀) carbocyclyl, 4- or 7-membered heterocyclyl, and wherein said alkyl, carbocyclyl, or heterocyclyl is optionally substituted; and
   A₁ is an active moiety as further defined herein below.

In some embodiments, R₁ is absent and R₁′ is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, 4- to 6-membered heterocyclyl, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —C(O)—. In some embodiments, the alkylene chain is interrupted by, and/or terminates (at either or both termini) with —C(O)O—. In some embodiments, the alkylene chain is interrupted by, and/or terminates (at either or both termini) with —C(O)N(R′)—. In some embodiments, the alkylene chain is interrupted by, and/or terminates (at either or both termini) with —N(R′)S(O)₂—. In some embodiments, the alkylene chain is interrupted by, and/or terminates (at either or both termini) with a 4- to 6-membered heterocyclyl. In some embodiments, the alkylene chain terminates with pyrrolidine-2,5-dione

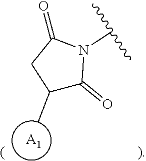

In some embodiments, R₁ is absent and R₁′ is a polyethylene glycol chain, which may be interrupted by, and/or terminate (at either or both termini) in at least one of —O—, —S—, —N(R′)—C≡C—, —C(O)—, —C(O)O—, —OC(O)—, —OC(O)O—, —C(NOR′)—, —C(O)N(R′)—, —C(O)N(R′)C(O)—R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the polyethylene glycol chain has 1 to 20 —(CH₂CH₂—O—)— units. In some embodiments, the polyethylene glycol chain has 1 to 15 —(CH₂CH₂—O—)— units. In some embodiments, the polyethylene glycol chain has 1 to 10 —(CH₂CH₂—O—)— units. In some embodiments, the polyethylene glycol chain has 1 to 5 —(CH₂CH₂—O—)— units. In some embodiments, the polyethylene glycol chain has 1 to 2 —(CH₂CH₂—O—)— units. In some embodiments, the polyethylene glycol is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, 4- to 6-membered heterocyclyl, or a combination thereof. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —C(O)—. In some embodiments, the polyethylene glycol chain is interrupted by, and/or terminates (at either or both termini) with —C(O)O—. In some embodiments, the polyethylene glycol chain is interrupted by, and/or terminates (at either or both termini) with —C(O)N(R′)—. In some embodiments, the polyethylene glycol chain is interrupted by, and/or terminates (at either or both termini) with —N(R′)S(O)₂—. In some embodiments, the polyethylene glycol chain is interrupted by, and/or terminates (at either or both termini) with 4- to 6-membered heterocyclyl. In some embodiments, the polyethylene glycol chain terminates with pyrrolidine-2,5-dione

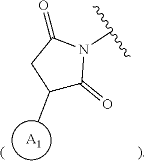

In some embodiments, R₁ and R₂, together with the nitrogen atom to which they are attached, form a 3- to 16-membered heterocyclyl containing 1-8 heteroatoms selected from N, O, and S. In some embodiments, R₁ and R₂, together with the nitrogen atom to which they are attached, form a 4- to 12-membered heterocyclyl containing 1-4 heteroatoms selected from N, O, and S. In some embodiments, R₁ and R₂, together with the nitrogen atom to which they are attached, form a 5- to 10-membered heterocyclyl containing 1-3 heteroatoms selected from N, O, and S. In some embodiments, R₁ and R₂, together with the nitrogen atom to which they are attached, form a 5- to 6-membered heterocyclyl containing 1-2 heteroatoms selected from N, O, and S. In some embodiments, R₁ and R₂, together with the nitrogen atom to which they are attached, form a piperazinyl group.

In some embodiments, R₁ is absent, R₁′ is a C₁-C₂₄ alkylene chain and R₂ is methyl, ethyl, isopropyl, or t-butyl. In some embodiments, R₁ is absent, R₁′ is a C₁-C₁₈ alkylene chain and R₂ is methyl, ethyl, isopropyl, or t-butyl. In some embodiments, R₁ is absent, R₁′ is a C1-C12 alkylene chain and R₂ is methyl, ethyl, isopropyl, or t-butyl. In some embodiments, R₁ is absent, R₁′ is a C₁-C₁₀ alkylene chain and R₂ is methyl, ethyl, isopropyl, or t-butyl. In some embodiments, R₁ is absent, R₁′ is a C₁-C₈ alkylene chain and R₂ is methyl, ethyl, isopropyl, or t-butyl. In some embodiments, R₁ is absent, R₁′ is a C₁-C₆ alkylene chain and R₂ is methyl, ethyl, isopropyl, or t-butyl. In some embodiments, R₁ is absent, R₁′ is a C₁-C₄ alkylene chain and R₂ is methyl, ethyl, isopropyl, or t-butyl. In some embodiments, R₁ is absent, R₁′ is a C₁-C₂ alkylene chain and R₂ is methyl, ethyl, isopropyl, or t-butyl. In some embodiments, R₁ is absent, R₁′ is 1 to 20 —(CH₂CH₂-0)— units and R₂ is methyl, ethyl, isopropyl, or t-butyl. In some embodiments, R₁ is absent, R₁′ is 1 to 15 —(CH₂CH₂—O—)— units and R₂ is methyl, ethyl, isopropyl, or t-butyl. In some embodiments, R₁ is absent, R₁′ is 1 to 10 —(CH₂CH₂—O—)— units and R₂ is methyl, ethyl, isopropyl, or t-butyl. In some embodiments, R₁ is absent, R₁′ is 1 to 5 —(CH₂CH₂—O—)— units and R₂ is methyl, ethyl, isopropyl, or t-butyl. In some embodiments, R₁ is absent, R₁′ is 1 to 2 —(CH₂CH₂—O—)— units and R₂ is methyl, ethyl, isopropyl, or t-butyl.

In some embodiments, the active moiety is a binding moiety. Representative examples of binding moieties include moieties that bind ubiquitin ligase enzymes or other cellular enzymes that catalyze degradation of cellular proteins. For example, the Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

In some embodiments, the binding moiety is a small molecule that binds the E3 ligase which is cereblon (CRBN). Representative examples of small molecules that bind CRBN are represented by any one of structures (D1-a) to (D1-d):

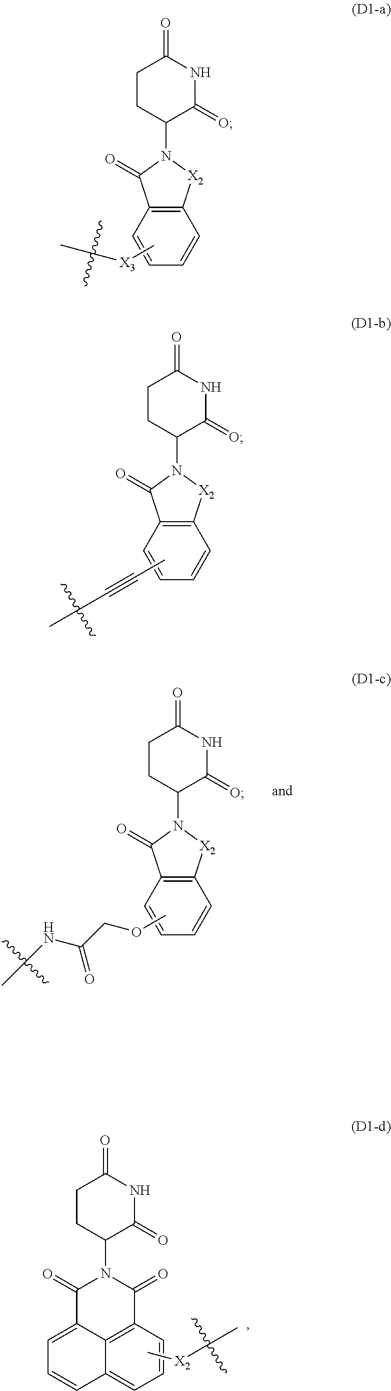

wherein X₂ is CH₂ or C(O) and X₃ is CR″₁R″₂, NR″₁, O, or S, wherein R″₁ and R″₂ are independently hydrogen, halogen, OH, NH₂, C₁-C₃ alkyl, C₁-C₃ alkoxy, or C₁-C₃ alkylamine, or R″₁ and R″₂, together with the atoms to which they are bound, form a C₃-C₇ carbocyclic or C3-C7 heterocyclic ring (e.g., azetidine, piperidine, pyrrolidine, cyclobutane, cyclohexane).

Yet other small molecules that bind cereblon and which may be suitable for use in the present invention are disclosed in U.S. Pat. No. 9,770,512, and U.S. Patent Application Publication Nos. 2018/0015087, 2018/0009779, 2016/0243247, 2016/0235731, 2016/0235730, and 2016/0176916, and International Patent Publications WO 2017/197055, WO 2017/197051, WO 2017/197036, WO 2017/197056 and WO 2017/197046.

In some embodiments, the binding moiety is a small molecule that binds the E3 ligase which is von Hippel-Lindau (VHL) tumor suppressor. Representative examples of small molecules that bind VHL are represented by any one of structures (D2-a) to (D2-j):

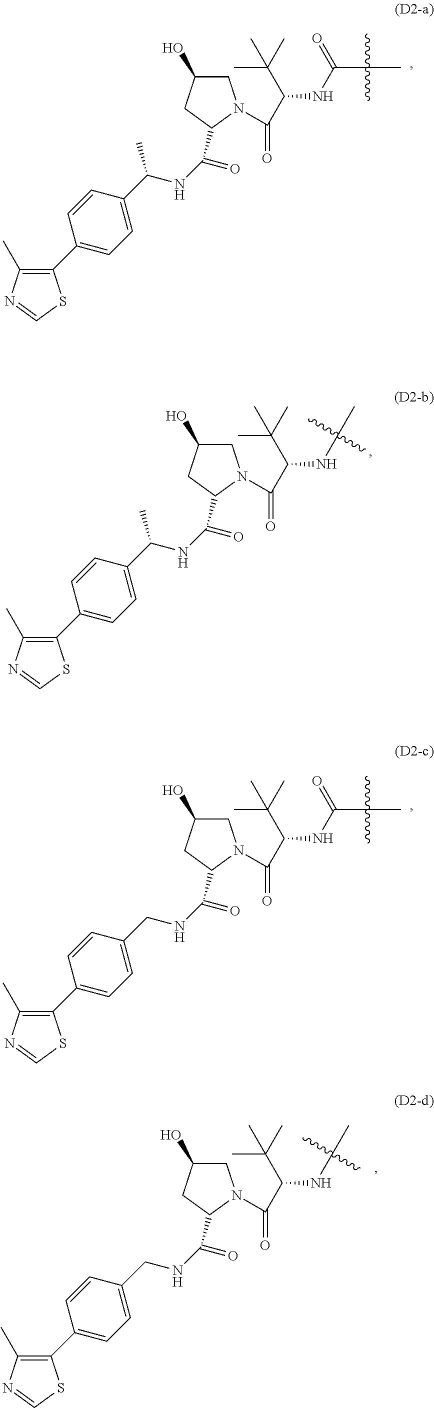

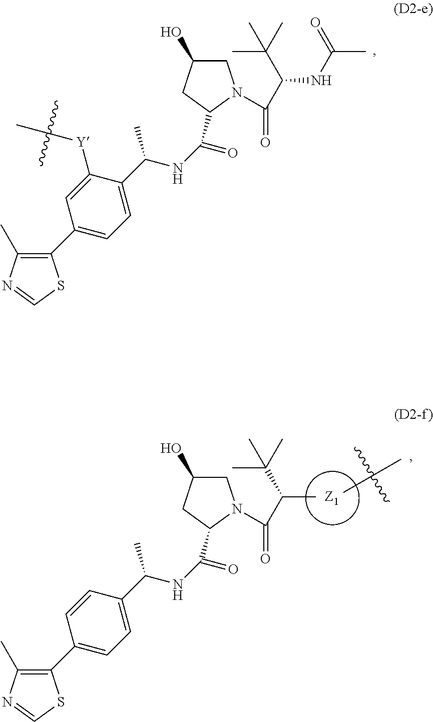

wherein Z₁ is a C₅-C₆ carbocyclic or C₅-C₆ heterocyclic group,

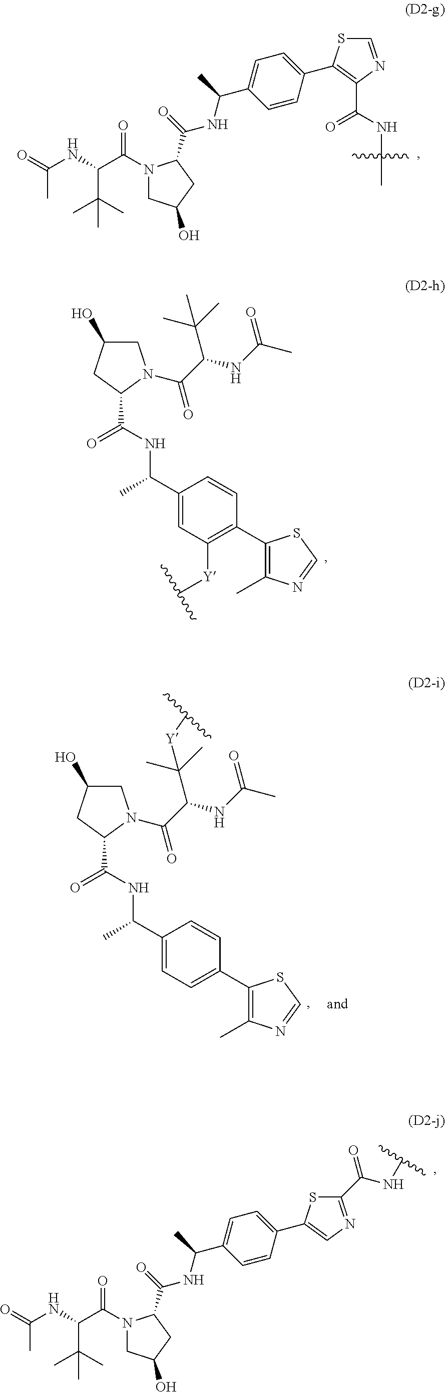

wherein Y′ is a bond, CH₂, NH, NMe, O, or S, or a stereoisomer thereof.

In some embodiments, Z₁ is phenyl, pyrrolyl, furanyl, thiophenyl, pyrazolyl, imidazolyl, oxazolyl, thiazolyl, pyridinyl, pyridazinyl, or pyrimidinyl. In certain embodiments, Z is

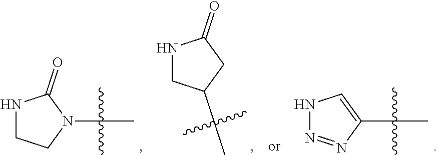 [SMILES: *N1CCNC1=O ; *C1CNC(=O)C1 ; *c1c[nH]nn1]

Yet other small molecules that bind VHL and which may be suitable for use in the present invention are disclosed in U.S. Patent Application Publication Nos. 2017/0121321 and 2014/0356322.

In some embodiments, the binding moiety is a small molecule that binds the E3 ligase which is an inhibitor of apoptosis protein (IAP). Representative examples of small molecules that bind IAP are represented by any one of structures (D3-a) to (D3-f).

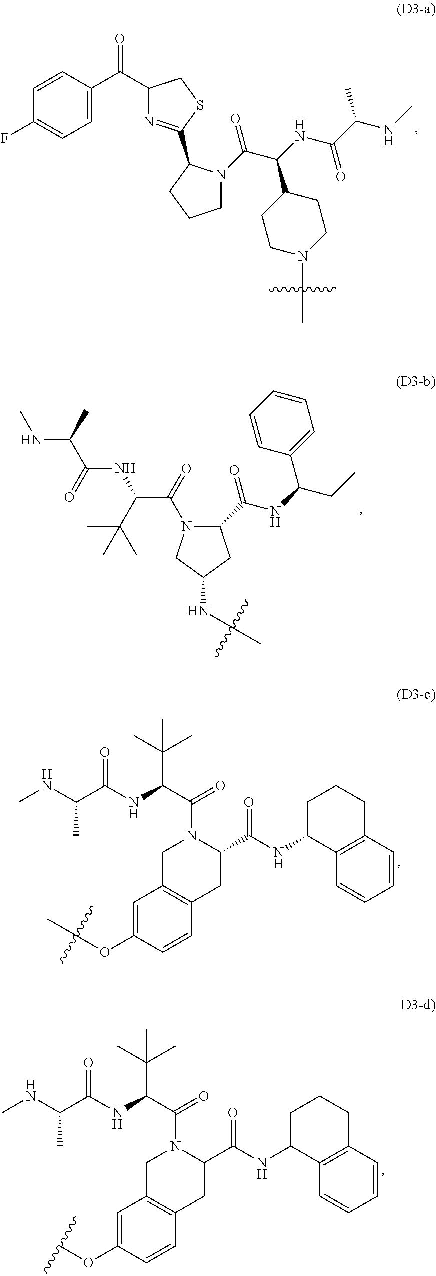

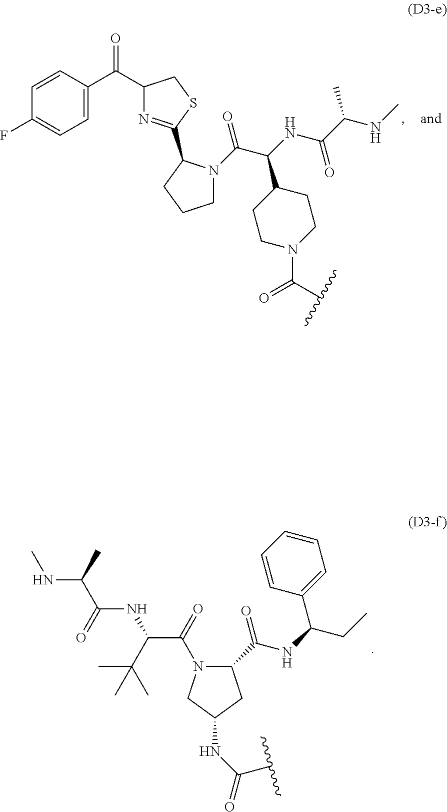

Yet other small molecules that bind IAP and which may be suitable for use in the present invention are disclosed in International Patent Application Publication Nos. WO 2008128171, WO 2008/016893, WO 2014/060768, and WO 2014/060767.

In some embodiments, the binding moiety is a small molecule that binds the E3 ligase which is murine double minute 2 (MDM2). Representative examples of small molecules that bind MDM2 are represented by structures (D4-a) and (D4-b):

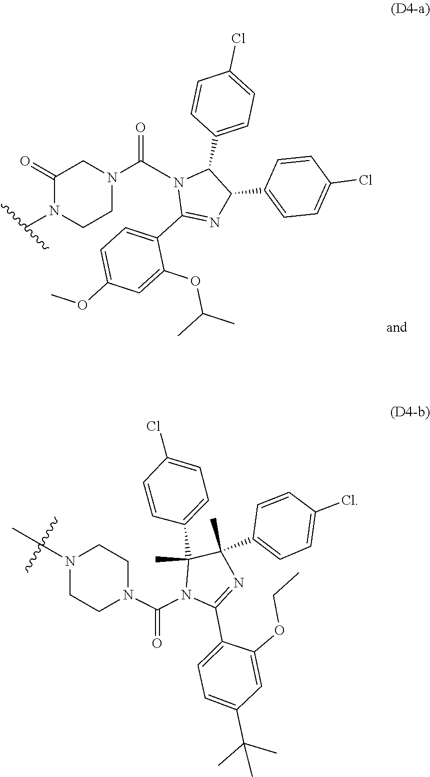

Yet other small molecules that bind MDM2 and which may be suitable for use in the present invention are disclosed in U.S. Pat. No. 9,993,472 B2. MDM2 is known in the art to function as an ubiquitin-E3 ligase.

In some embodiments, the binding moiety is a small molecule that binds the ubiquitin receptor RPN13. Representative examples of small molecules that bind RPN13 are represented by structures (D5-a), (D5-b), (D5-c), and (D5-d):

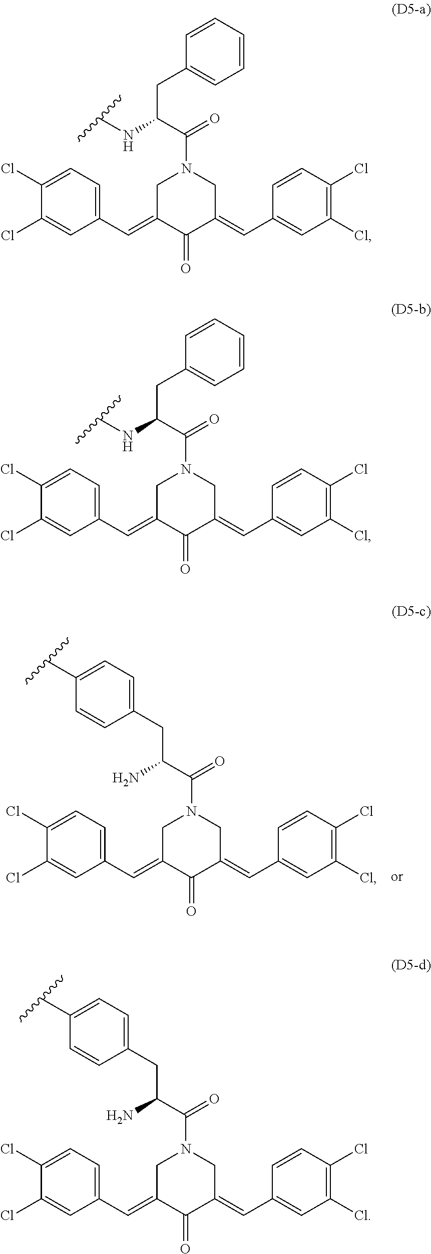 [SMILES: *N[C@H](Cc1ccccc1)C(=O)N1C/C(=C\c2ccc(Cl)c(Cl)c2)C(=O)/C(=C/c2ccc(Cl)c(Cl)c2)C1 ; *N[C@@H](Cc1ccccc1)C(=O)N1C/C(=C\c2ccc(Cl)c(Cl)c2)C(=O)/C(=C/c2ccc(Cl)c(Cl)c2)C1 ; *c1ccc(C[C@@H](N)C(=O)N2C/C(=C\c3ccc(Cl)c(Cl)c3)C(=O)/C(=C/c3ccc(Cl)c(Cl)c3)C2)cc1 ; "or" ; *c1ccc(C[C@H](N)C(=O)N2C/C(=C\c3ccc(Cl)c(Cl)c3)C(=O)/C(=C/c3ccc(Cl)c(Cl)c3)C2)cc1]

Yet other small molecules that bind RPN13 and which may be suitable for use in the present invention are disclosed in International Publication No. PCT/US2020/012825. RPN13 is known in the art to function as an ubiquitin receptor.

In some embodiments, A₁ is a binding moiety that binds a cellular protein other than a cellular enzyme that catalyzes degradation of cellular proteins (such as ubiquitin ligases). Representative examples of cellular proteins that may be targeted by inventive compounds that contain a binding moiety include kinases, BET bromodomain-containing protein, cytosolic signaling proteins (e.g., FKBP12), nuclear proteins, histone deacetylases (HDAC), lysine methyltransferase, aryl hydrocarbon receptors (AHR), estrogen receptors, androgen receptors, glucocorticoid receptors, and transcription factors (e.g., SMARCA4, SMARCA2, TRIM24).

In certain embodiments, the binding moiety binds a tyrosine kinase (e.g., AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFR3, FGFR4, FGR, FLT1, FLT3, FLT4, FRK, FYN, GSG2, HCK, IGF1R, ILK, INSR, INSRR, IRAK4, ITK, JAK1, JAK2, JAK3, KDR, KIT, KSR1, LCK, LMTK2, LMTK3, LTK, LYN, MATK, MERTK, MET, MLTK, MST1R, MUSK, NPR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TNK2, TNNI3K, TXK, TYK2, TYRO3, YES1, or ZAP70), a serine/threonine kinase (e.g., casein kinase 2, protein kinase A, protein kinase B, protein kinase C, Raf kinases, CaM kinases, AKT1, AKT2, AKT3, ALK1, ALK2, ALK3, ALK4, Aurora A, Aurora B, Aurora C, CHK1, CHK2, CLK1, CLK2, CLK3, DAPK1, DAPK2, DAPK3, DMPK, ERK1, ERK2, ERK5, GCK, GSK3, HIPK, KHS1, LKB1, LOK, MAPKAPK2, MAPKAPK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PIM2, PLK1, RIP2, RIP5, RSK1, RSK2, SGK2, SGK3, SIK1, STK33, TAO1, TAO2, TGF-beta, TLK2, TSSK1, TSSK2, ULK1, or ULK2), a cyclin dependent kinase (e.g., Cdk1-Cdk11), or a leucine-rich repeat kinase (e.g., LRRK2).

In certain embodiments, the binding moiety binds a bromodomain and extra terminal (BET) protein, representative examples of which include ATPase family AAA domain-containing protein 2 (ATAD2), bromodomain adjacent to zinc finger domain protein 1A (BAZ1A), BAZ1B, BAZ2A, BAZ2B, bromodomain containing protein 1 (BRD1), BRD2, BRD3, BRD4, BRD5, BRD6, BRD7, BRD8, BRD9, BRD10, bromodomain testis-specific protein (BRDT), romodomain and PHD finger-containing protein 1 (BRPF1), BRPF3, bromodomain And WD Repeat Domain Containing 3 (BRWD3), cat eye syndrome critical region protein 2 (CECR2), CREB binding protein (CREBBP), E1A binding protein P300 (EP300), general control of amino-acid synthesis 5-like 2 (GCN5L2), histone-lysine N-methyltransferase 2A (KMT2A), P300/CBP-associated factor (PCAF), PH-interacting protein (PHIP), protein kinase C binding protein 1 (PRKCBP1), SWI/SNF Related, Matrix Associated, Actin Dependent Regulator Of Chromatin, Subfamily A, Member 2 (SMARCA2), SMARCA4, Sp100 nuclear body protein (SP100), SP110, SP140, transcription initiation factor TFIID subunit 1 (TAF1), TAF1L, TIF1a, tripartite motif-containing 28 (TRIM28), TRIM33, TRIM66, WD repeat protein 9 (WDR9), zinc finger MYND domain-containing protein 11 (ZMYND11), and mixed lineage leukemia-like protein 4 (MLL4). In certain embodiments, the BET bromodomain-containing protein is BRD4.

In certain embodiments, the binding moiety binds to BRD2, BRD3, BRD4, Antennapedia Homeodomain Protein, BRCA1, BRCA2, a CCAAT-Enhanced-Binding Protein, histone, a Polycomb-group protein, a High Mobility Group Protein, a Telomere Binding Protein, FANCA, FANCD2, FANCE, FANCF, HDAC1, HDAC2, HDAC3, HDAC4, HDAC5, HDAC6, HDAC7, HDAC8, HDAC9, HDAC10, HDAC11, a hepatocyte nuclear factor, Mad2, NF-kappa B, a Nuclear Receptor Coactivator, CREB-binding protein, p55, p107, p130, p53, c-fos, c-jun, c-mdm2, c-myc, or c-rel.

In some embodiments, the binding moiety binds to BRD. Representative examples of small molecules that bind BRD include:

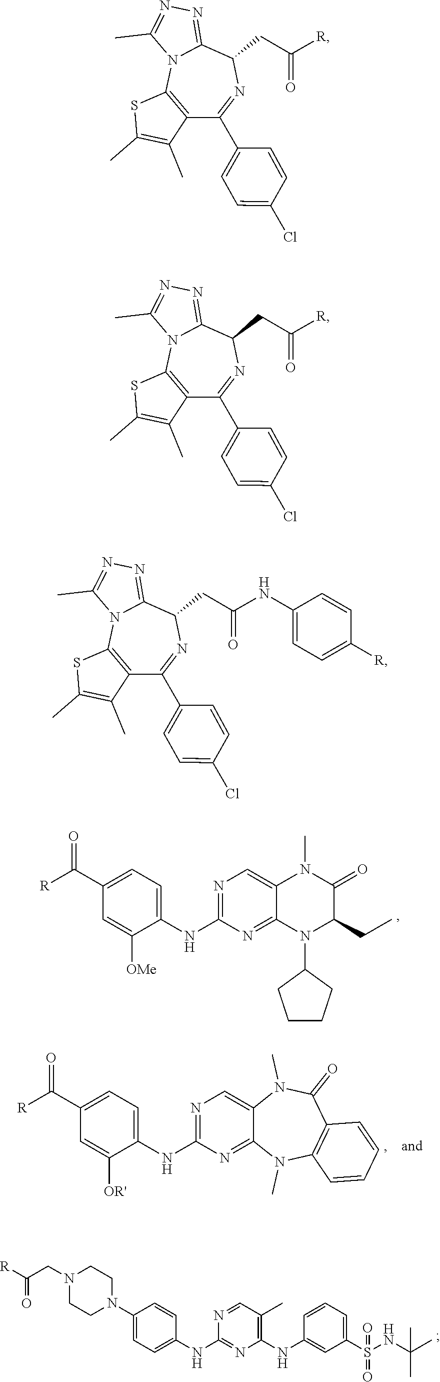

wherein:
R is the point at which the linking group is attached; and
R′ is methyl or ethyl.

In some embodiments, the binding moiety binds to CREBBP. Representative examples of small molecules that bind CREBBP include:

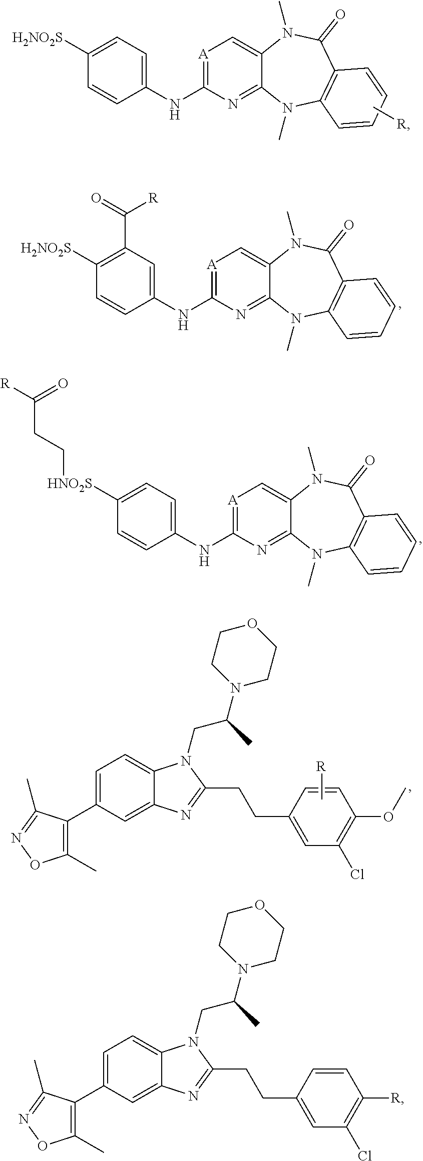

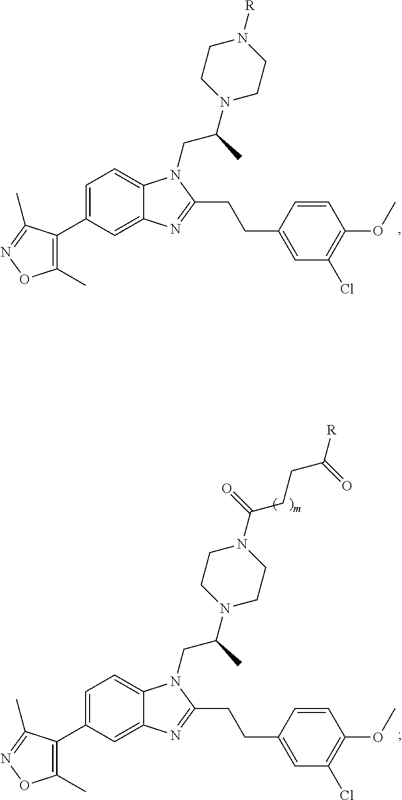

wherein:
R is the point at which the linking group is attached;
A is N or CH; and
m is 0, 1, 2, 3,4, 5, 6,7, or 8.

In some embodiments, the binding moiety binds to SMARCA4/PB1/SMARCA2. Representative examples of small molecules that bind SMARCA4/PB1/SMARCA2 include:

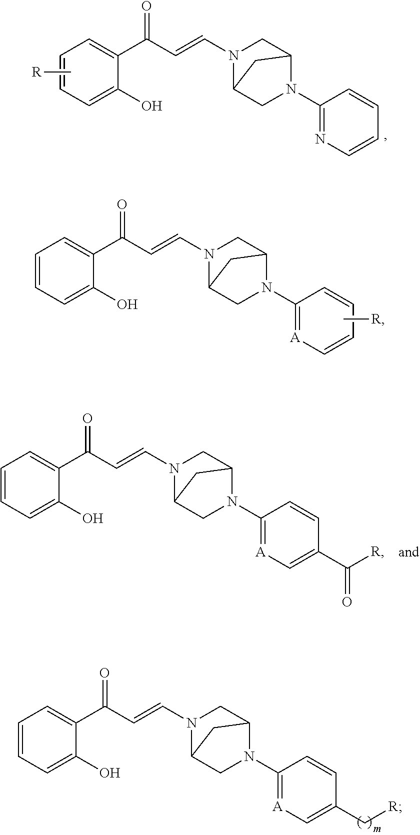

wherein:
R is the point at which the linking group is attached;
A is N or CH; and
m is 0, 1, 2, 3, 4, 5, 6, 7, or 8.

In some embodiments, the binding moiety binds to TRIM24/BRPF1. Representative examples of small molecules that bind TRIM24/BRPF1 include:

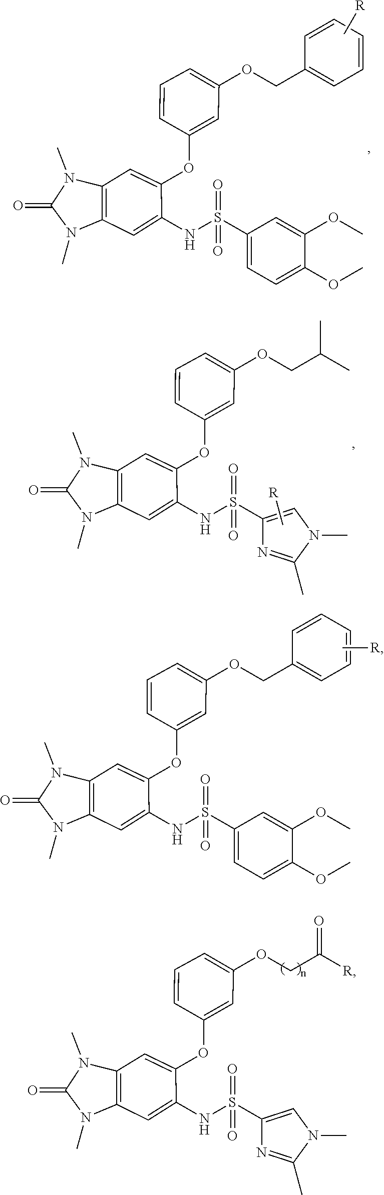

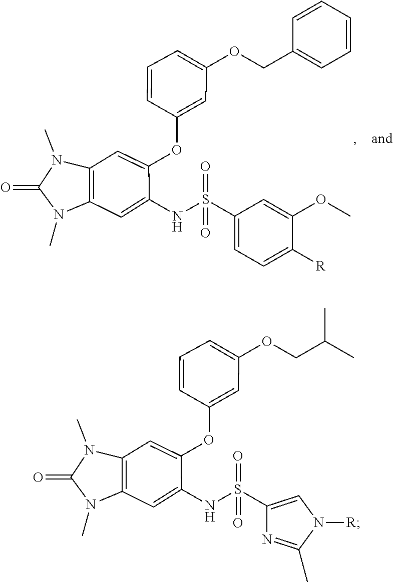

wherein:
R is the point at which the linking group is attached; and
m is 0, 1, 2, 3, 4, 5, 6, 7, or 8.

In some embodiments, the binding moiety binds to a glucocorticoid receptor.

Representative examples of small molecules that bind a glucocorticoid receptor include:

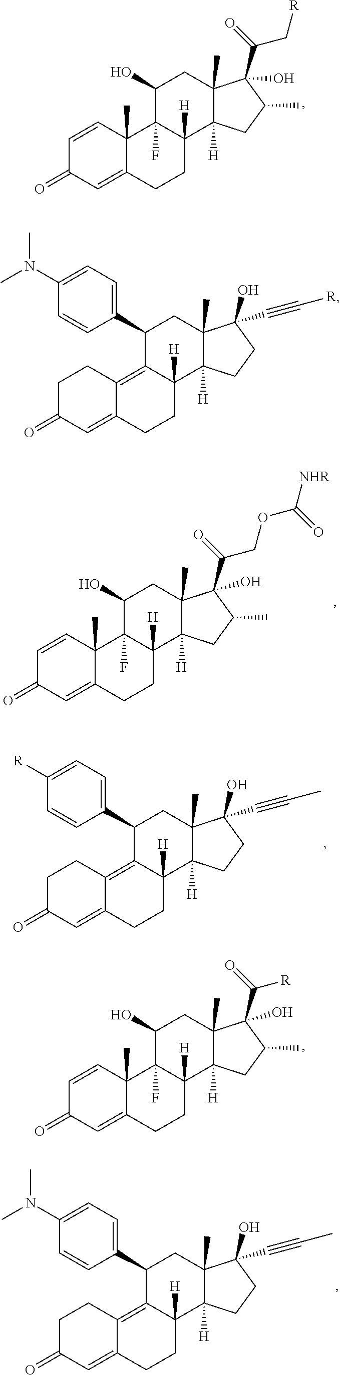

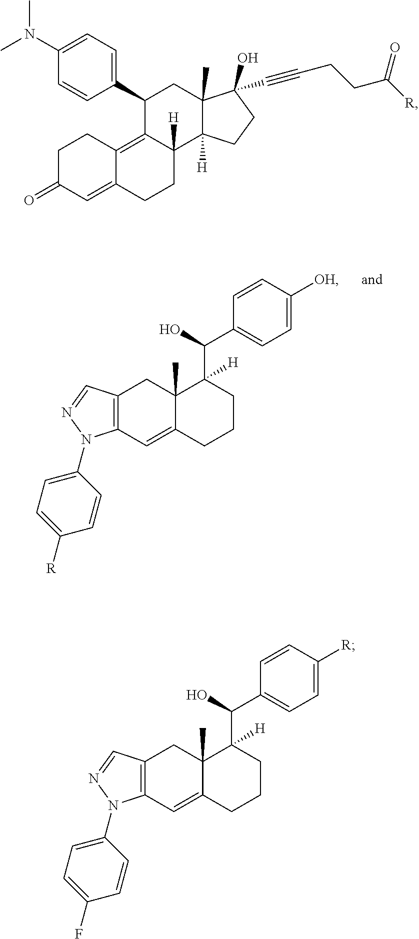

wherein:
R is the point at which the linking group is attached.

In some embodiments, the binding moiety binds to an estrogen/androgen receptor. Representative examples of small molecules that bind an estrogen/androgen receptor include:

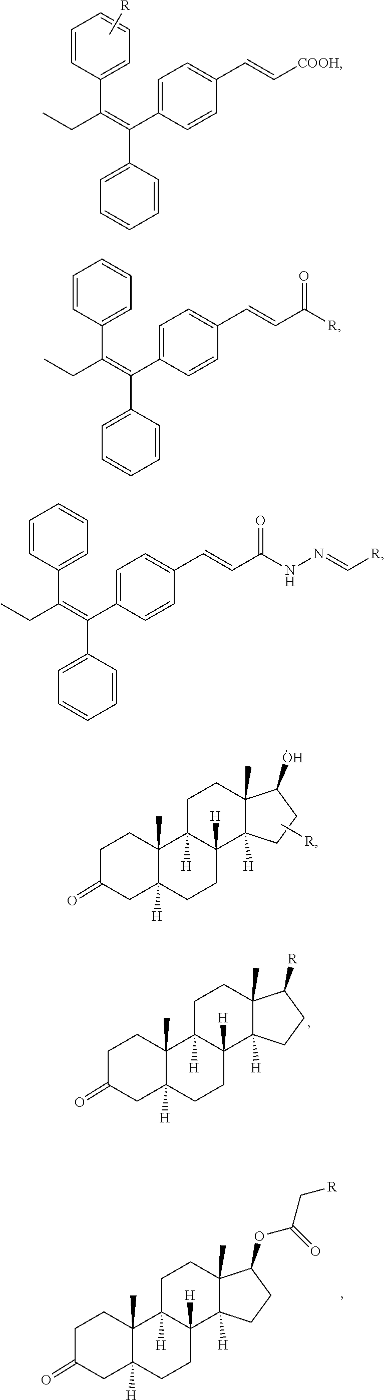

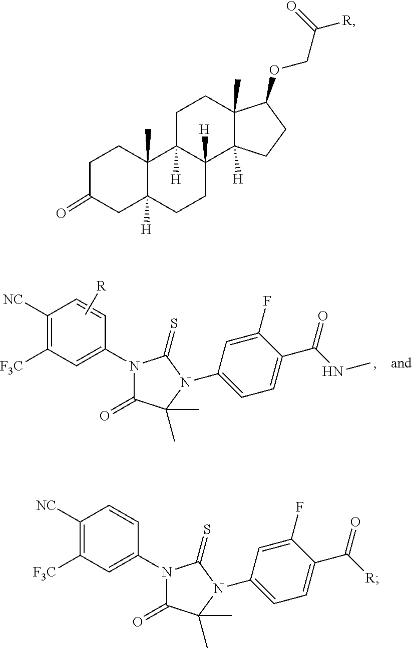

wherein:
R is the point at which the linking group is attached.

In some embodiments, the binding moiety binds to DOT1L. Representative examples of small molecules that bind DOT1L:

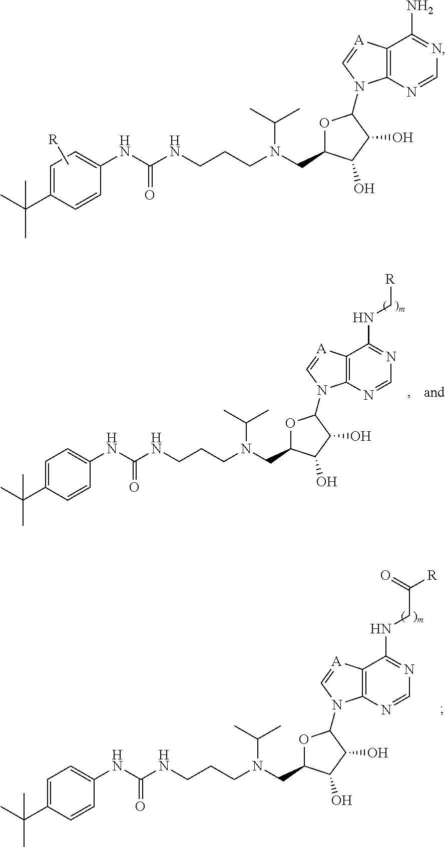

wherein:
R is the point at which the linking group is attached;
A is N or CH; and
m is 0, 1, 2, 3, 4, 5, 6, 7, or 8.

In some embodiments, the binding moiety binds to Ras. Representative examples of small molecules that bind Ras:

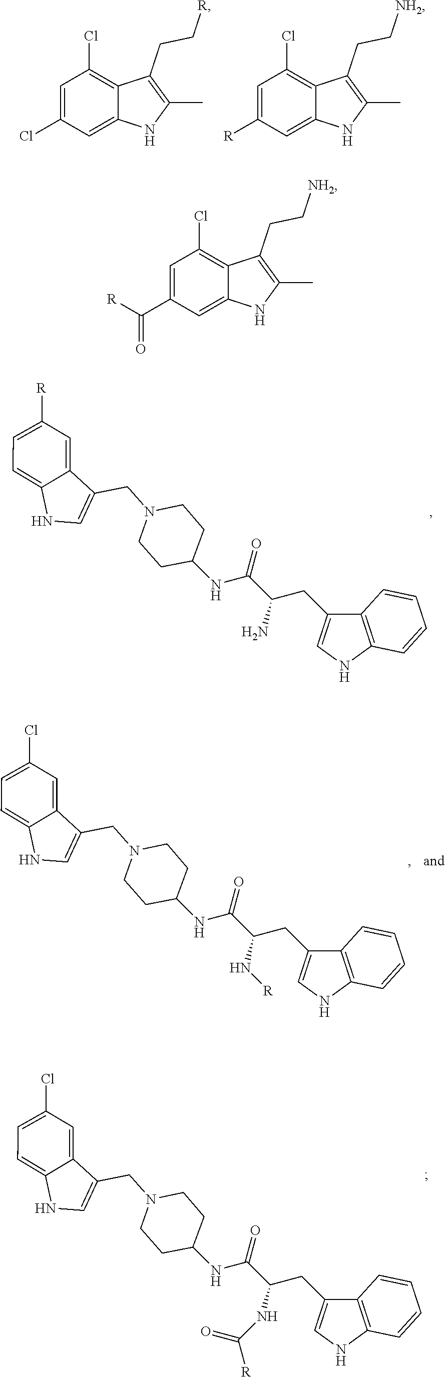

wherein:
R is the point at which the linking group is attached.

In some embodiments, the binding moiety binds to RasG12C. Representative examples of small molecules that bind RasG12C:

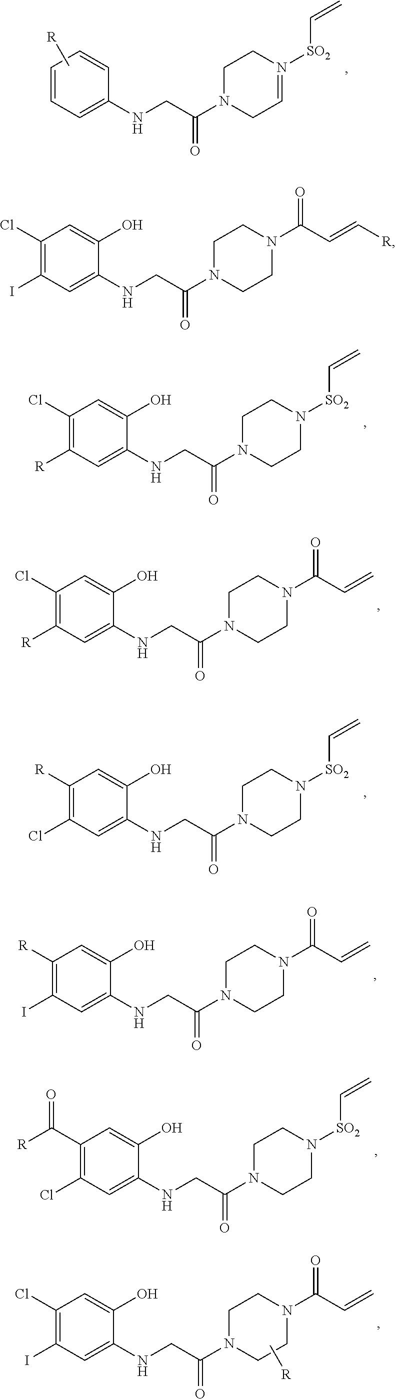

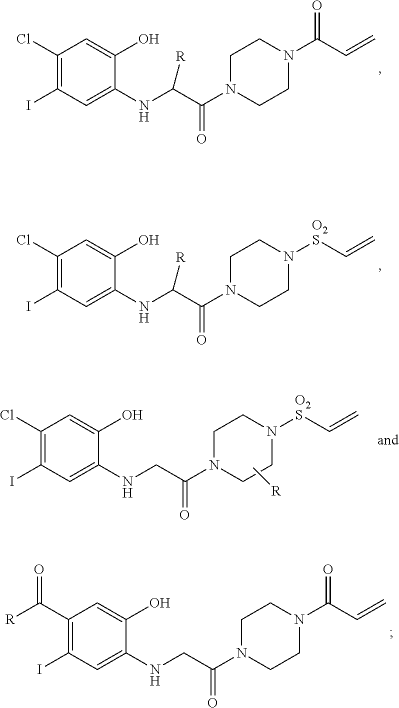

wherein:
R is the point at which the linking group is attached.

In some embodiments, the binding moiety binds to Her3. Representative examples of small molecules that bind Her3:

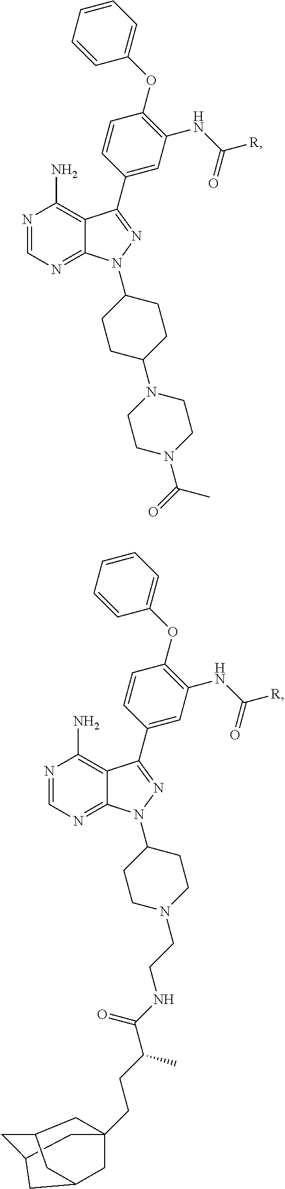

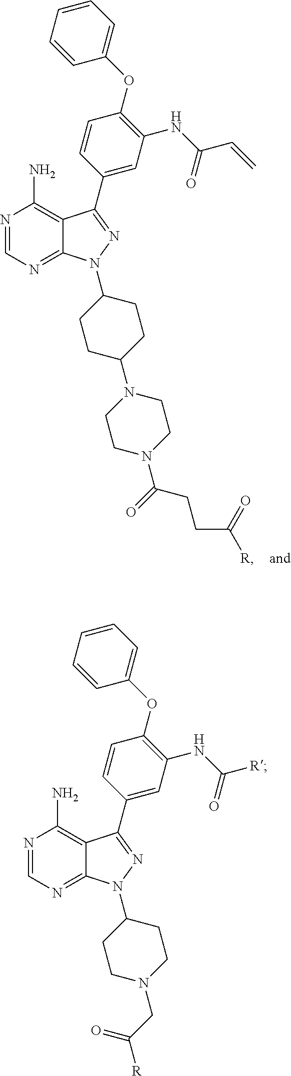

wherein:
R is the point at which the linking group is attached; and
R′ is

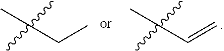

In some embodiments, the binding moiety binds to Bcl-2/Bcl-XL. Representative examples of small molecules that bind Bcl-2/Bcl-XL:

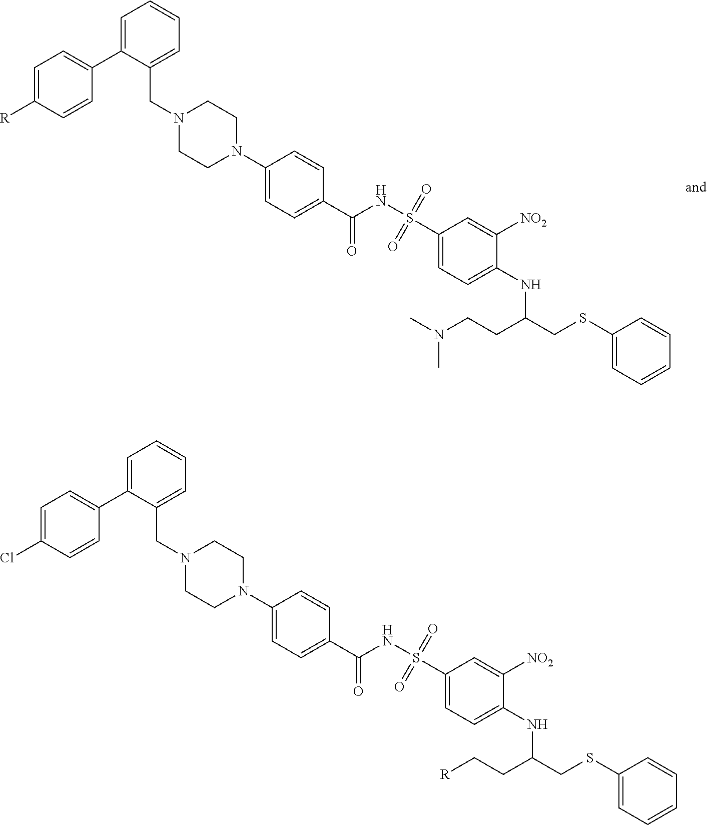

wherein:
R is the point at which the linking group is attached.

In some embodiments, the binding moiety binds to HDAC. Representative examples of small molecules that bind HDAC:

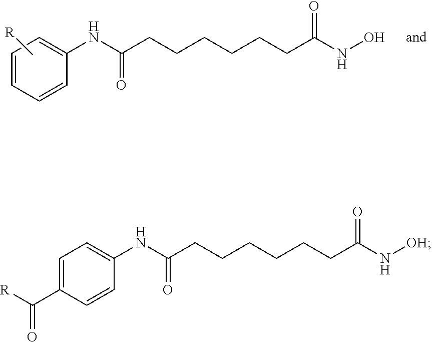

wherein:
R is the point at which the linking group is attached.

In some embodiments, the binding moiety binds to PPAR-gamma. Representative examples of small molecules that bind PPAR-gamma:

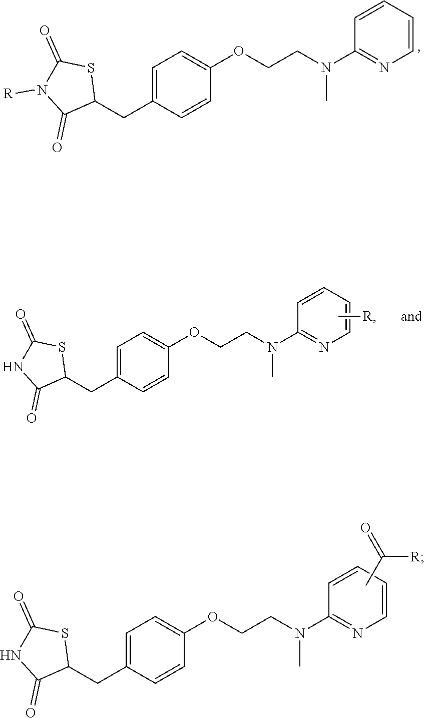

wherein:
R is the point at which the linking group is attached.

In some embodiments, the binding moiety binds to RXR. Representative examples of small molecules that bind RXR:

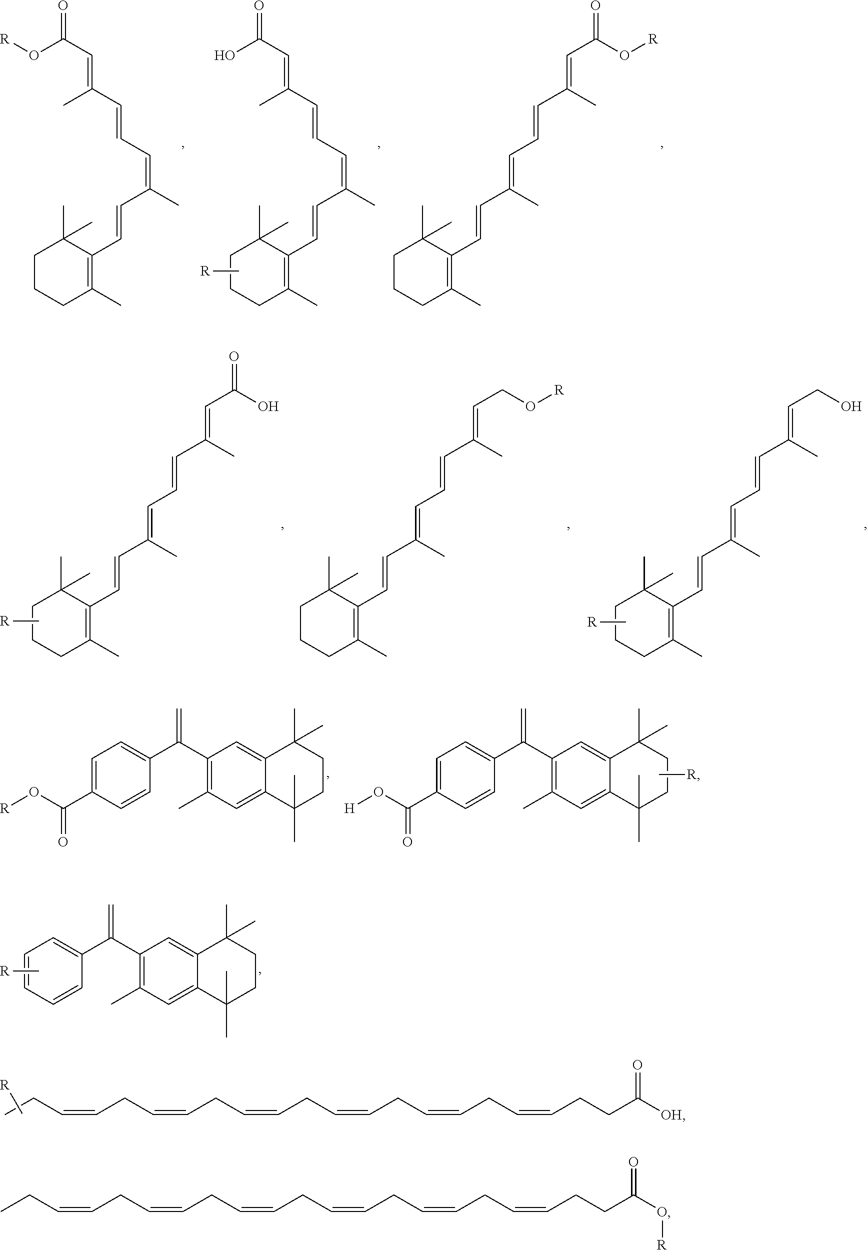

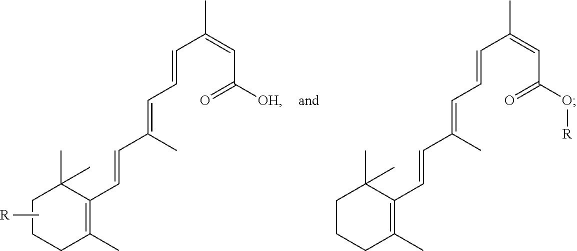

wherein:
R is the point at which the linking group is attached.

In some embodiments, the binding moiety binds to DHFR. Representative examples of small molecules that bind DHFR:

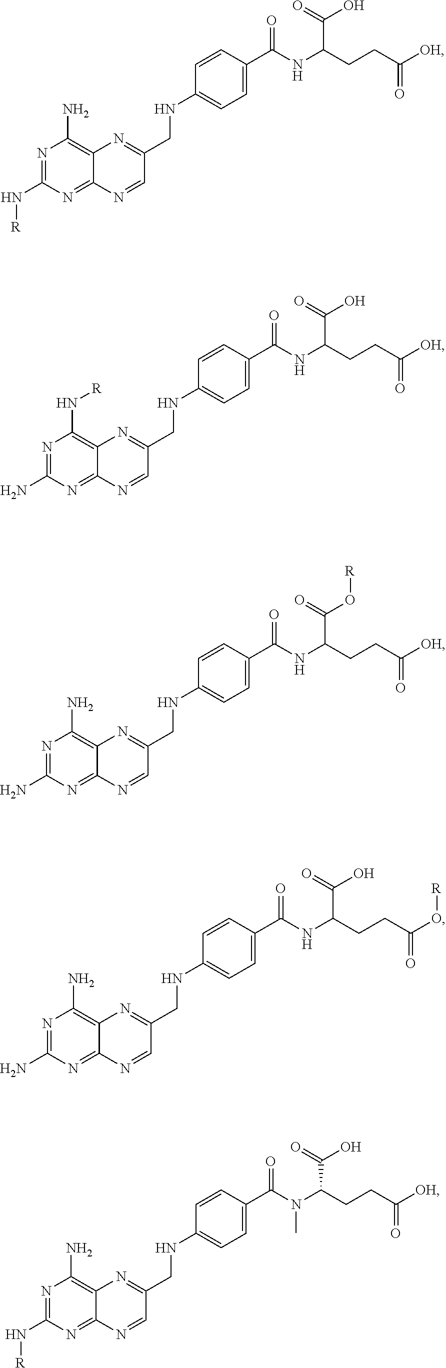

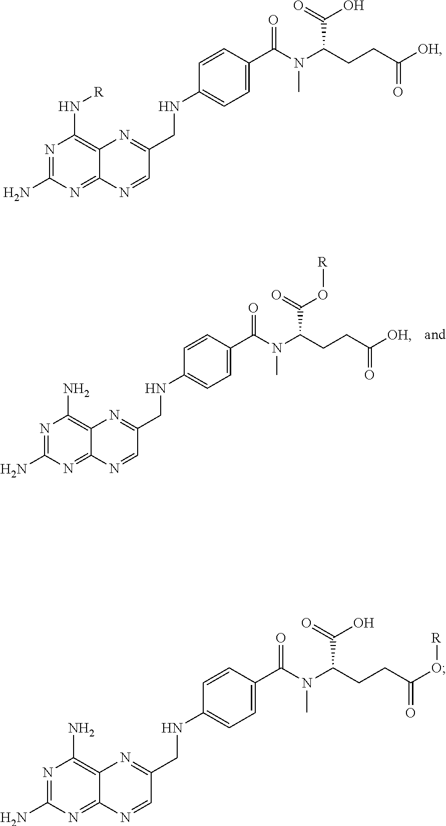

wherein:
R is the point at which the linking group is attached.

In some embodiments, the binding moiety binds to BCL2. Representative examples of small molecules that bind BCL2:

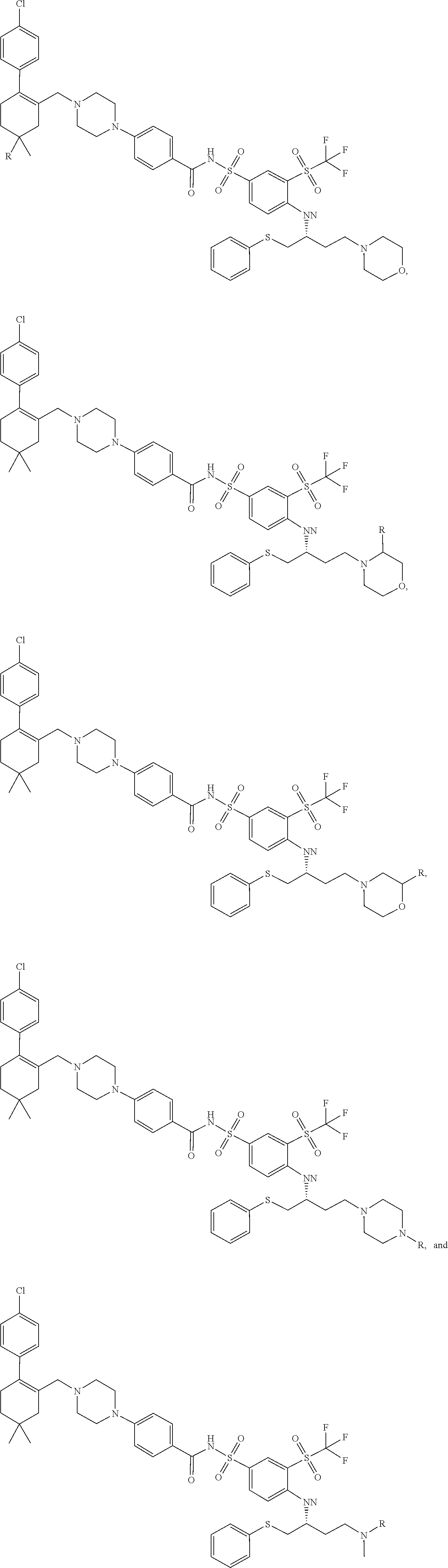

wherein:
R is the point at which the linking group is attached.

Yet other small molecules that bind cellular proteins and which may be suitable for use as binding moieties in the present invention are disclosed in U.S. Patent Application Publication Nos. 2017/0121321 and 2014/0356322.

In some embodiments, the binding moiety is biotin or a biotin derivative. Biotin derivatives are known in the art. See, e.g., Molecular Probes Handbook, A Guide to Fluorescent Probes and Labeling Technologies, 11^{th} Ed., Life Technologies Corporation, 2010. Biotin and its derivatives have been widely used as molecular labels in the biotechnology industry for many years. Representative examples of biotin derivatives that may be suitable for use in the present invention include desthiobiotin, pyrimethamine biotin, rac selenobiotin, biocytin, 2-iminobiotin, biocytin-L-proline, biotinyl cystamine, and biotinyl tobramycin amide. Other biotin derivatives that may be suitable for use in the present invention are described in the art, e.g., U.S. Pat. No. 8,318,696 and U.S. Patent Application Publication No. 2007/0020206, each of which is incorporated by reference.

In some embodiments, the binding moiety is short peptide sequence (e.g., 2 to 50 amino acids in length, e.g., 4 to 20 amino acids in length, wherein the amino acid residues in the peptide may be the same or different). Representative examples include α-amanitin, antipain, ceruletide, glutathione, leupeptin, netropsin, pepstatin, peptide T, phalloidin, teprotide, tuftsin, ALFA-tag, AviTag, C-tag, calmodulin-tag, polyglutamate tag, poly arginine tag, E-tag, FLAG-tag, HA-tag, His-tag, Myc-tag, NE-tag, Rho1D4-tag, S-tag, SBP-tag, softag 1, softag 3, Spot-tag, Strep-tag, T7-tag, TC tag, Ty tag, V5 tag, VSV-tag, and Xpress tag.

In some embodiments, the binding moiety is a protein. Representative examples of proteinaceous binding moieties include chitin binding protein (CBP), maltose binding protein (MBP), glutathione-S-transferase (GST), thioredoxin, poly(NANP), biotin carboxyl carrier protein (BCCP), green fluorescent protein (GFP), HaloTag, SNAP-tag, CLIP-tag, HUH-tag, Nus-tag, Fc-tag, and carbohydrate recognition domain-tag. In some embodiments, the binding moiety is a HaloTag.

In yet other embodiments, the binding moiety is an antibody (e.g., a monoclonal antibody) or a fragment thereof that binds an intended target. In some embodiments, the monoclonal antibody binds a cell surface receptor present on a diseased cell. In some embodiments, the monoclonal antibody binds a tumor associated antigen on a cancer cell such as a solid tumor cell. Representative examples of monoclonal antibodies include muromonab-CD3, abciximab, rituximab, palivizumab, infliximab, trastuzumab, alemtuzumab, adalimumab, ibritumomab, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab, pertuzumab, raxibacumab, obinutuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab, brodalumab, guselkumab, dupilumab, sarilumab, avelumab, ocrelizumab, emicizumab, benralizumab, gemtuzumab, durvalumab, burosumab, lanadelumab, mogamulizumab, erenumab, galcanezumab, tildrakizumab, cemiplimab, emapalumab, fremanezumab, ibalizumab, moxetumomab, ravulizumab, caplacizumab, romosozumab, risankizumab, polatuzumab, brolucizumab, crizanlizumab, sacituzumab, belantamab, or enfortumab or a fragment thereof. In some embodiments, the monoclonal antibody or binding fragment thereof is gemtuzumab, brentuximab, trastuzumab, inotuzumab, moxetumomab, polatuzumab, enfortumab, or belantamab.

In some embodiments, the binding moiety is a fragment of an antibody e.g., a monoclonal antibody. For example, the fragment may be a variable fragment such as a single chain variable fragment (scFv) of the monoclonal antibody. Representative examples of scFvs include pexelizumab, duvortuxizumab, efungumab, gancotamab, letolizumab, oportuzumab monatox, vobarilizumab, and brolucizumab.

In some embodiments, the active moiety is a binding moiety which is a solubility enhancing group. Examples of solubilizing groups include substituents containing a group succeptible to being ionized in water at a pH range from 0 to 14, ionizable groups capable of forming salts, and highly polar substituents having a high dipolar moment and capable of forming strong interaction with water molecules. In some embodiments, the solubility enhancing group is alpha-chloro acetyl.

In some embodiments, the active moiety is a therapeutic moiety. The therapeutic moiety may, in some embodiments, be a small molecule. In certain embodiments, the molecular weight of the small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of the small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. In certain embodiments, therapeutic moiety is a therapeutically active agent such as a drug (e.g., a molecule approved by the U.S. Food and Drug Administration as provided in the Code of Federal Regulations (C.F.R.)).

In some embodiments, the therapeutic moiety is an anti-cancer agent. Representative types of anti-cancer agents include anti-angiogenic agents, alkylating agents, antimetabolites, microtubulin polymerization perturbers, platinum coordination complexes, anthracenediones, substituted ureas, methylhydrazine derivatives, adrenocortical suppressants, hormones and antagonists, anti-cancer polysaccharides and anthracycline (e.g., an aclarubicin, daunorubicin, doxorubicin, epirubicin, idarubicin, mitoxantrone, pirarubicin, valrubicine and derivatives and analogs thereof), and kinase inhibitors (e.g., pan-Her inhibitors (e.g., HKI-272, BIBW-2992, PF299, SN29926 and PR-509E)).

In some embodiments, the therapeutic moiety is a non-targeted cancer agent, which as known in the art refers to agents with relatively broad modes of action. Representative examples of non-targeted anti-cancer agents include alkylating agents (e.g., busulfan, chlorambucil, cyclophosphamide, ifosfamide, mechlorethamine, melphalan, carmustine, streptozocin, dacarbazine, temozolomide, altretamine, and thioTEPA), antimetabolites (e.g., capecitabine, cytarabine, 5′-fluorouracil, gemcitabine, cladribine, fludarabine, 6-mercaptopurine, and pentostatin), folate antagonists (e.g., methotrexate and pemetrexed), mitotic inhibitors (e.g., ocetaxel, paclitaxel, vinblastine, vincristine, vindesine, and vinorelbine), DNA inhibitors (e.g., hydroxyurea, carboplatin, cisplatin, oxaliplatin, mitomycin C, and pyrrolobenzodiazepine), topoisomerase inhibitors (e.g., topotecan, irinotecan, daunorubican, doxorubicin, etoposide, teniposide, and mitoxantrone), inducers of DNA breaks (e.g., bleomycin), ozogamicin, vedotin, emtansine, pasudotox, deruxtecan, govitecan, and mafodotin, or derivatives thereof.

In some embodiments, the therapeutic moiety is a targeted anti-cancer agent, which as known in the art, refers to agents with specific modes of action. Representative examples of non-targeted anti-cancer agents include afatinib (EGFR, HER2), axitinib (KIT, PDGFRP, VEGFR1/2/3), bosutinib (ABL), cabozantinib (FLT3, KIT, MET, RET, VEGFR2), ceritinib (ALK), crizotinib (ALK, MET), dabrafenib (ABL), erlotinib (EGFR), ibrutinib (BTK), idelalisib (PI3K6), imatinib (KIT, PDGFR, ABL), lapatinib (HER2, EGFR), lenvatinib (VEGFR2), nilotinib (ABL), olaparib (PARP), palbociclib (CDK4, CDK6), panobinostat (HDAC), pazopanib (VEGFR, PDGFR, KIT), ponatinib (ABL, FGFR1-3, FLT3, VEGFR2), regorafenib (KIT, PDGFRP, RAF, RET, VEGFR1/2/3), romidepsin (HDAC), ruxolitinib (JAK1/2), sorafenib (VEGFR, PDGFR, KIT, RAT), temsirolimus (mTOR), trametinib (MEK), vandetanib (EGFR, RET, VEGFR2), vemurafenib (BRAF), vismodegib (PTCH), and vorinostat (HDAC). In some embodiments, the targeted anti-cancer agent is a kinase inhibitor. Representative examples of kinase inhibitors include abemaciclib, acalabrutinib, afatinib, alectinib, avapritinib, axitinib, baricitinib, benimetinib, bosutinib, brigatinib, cabozantinib, ceritinib, capmatinib, cobimetinib, crizotinib, dabrafenib, dacomitinib, dasatinib, encorafenib, entrectinib, erdafitinib, erlotinib, everolimus, fedratinib, fostamatinib, gefitinib, gilteritinib, ibrutinib, icotinib, imatinib, lapatinib, larotrectinib, lenvatinib, lorlatinib, midostaurin, neratinib, netarsudil, nilotinib, nintedanib, osimertinib, palbociclib, pazopanib, pemigatinib, pexidartinib, ponatinib, pralsetinib, regorafenib, ribociclib, ripretinib, ruxolitinib, selpercatinib, selumetinib, sirolimus, sorafenib, sunitinib, temsirolimus, tofacitinib, tremetinib, tucatinib, upadacitinib, vandetanib, vemurafenib, and zanubrutinib.

In some embodiments, the therapeutic moiety is an anti-bacterial agent. Representative examples of antibacterial agents include plazomicin, eravacycline, sarecycline, omadacycline, rifamycin, imipenem, cilastatin, relebactam, pretomanid, lefamulin, cefiderocol, sulfaquinoxaline, oxytetracycline, hygromycin B, tylosin, chlortetracycline, virginiamycin, neomycin, luncomycin, pyrantel, melengestrol, lasalocid, fenbendazole, semduramicin, decoquinate, ractopamine, laidlomycin, diclazuril, halifuginone, robenidine, clopidol, zilpaterol, monensin, zoalene, lubabegron, and bacitracin.

In some embodiments, the therapeutic moiety is a non-steroidal anti-inflammatory drug (NSAID). Representative examples of NSAIDs agents include celecoxib, diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, sulindac, and tolmetin.

In some embodiments, the therapeutic moiety is a corticosteroid. Representative examples of corticosteroids agents include deflazacort, dexamethasone, betamethasone, triamcinolone, hydrocortisone, methylprednisolone and prednisone.

In some embodiments, the therapeutic moiety is a disease-modifying antirheumatic drug (DMARD). Representative examples of DMARDs include hydroxychloroquine, leflunomide, methotrexate, sulfasalazine, minocycline, penicillamine, cyclophosphamide, azathiopurine, cyclosporine, apremilast, and mycophenolate mofetil.

In some embodiments, the active moiety is a diagnostic moiety. Diagnostic moieties typically contain a detectable moiety such as a label. Representative examples of diagnostic moieties include dyes, chromogenic agents, positron emission tomography (PET) tracers, and magnetic resonance imaging (MRI) contrast agents. The term “label” includes any moiety that allows the compound to which it is attached to be captured, detected, or visualized. A label may be directly detectable (i.e., it does not require any further reaction or manipulation to be detectable, e.g., a fluorophore or chromophore is directly detectable) or it may be indirectly detectable (i.e., it is made detectable through reaction with or binding to another entity that is detectable, e.g., a hapten is detectable by immunostaining after reaction with an appropriate antibody comprising a reporter such as a fluorophore). Representative examples of types of labels include affinity tags, radiometric labels (e.g., radionuclides (such as, for example, ³²P ³⁵S, ³H, ¹⁴C, ¹²⁵I, ¹³¹I, and the like)), fluorescent dyes, phosphorescent dyes, chemiluminescent agents (such as, for example, acridinium esters, stabilized dioxetanes, and the like), spectrally resolvable inorganic fluorescent semiconductor nanocrystals (i.e., quantum dots), metal nanoparticles (e.g., gold, silver, copper, and platinum) or nanoclusters, enzymes (such as, for example, those used in an ELISA, i.e., horseradish peroxidase, beta-galactosidase, luciferase, alkaline phosphatase), colorimetric labels (such as, for example, dyes, colloidal gold, and the like), magnetic labels (such as, for example, Dynabeads™), and haptens.

In certain embodiments, the label comprises a fluorescent dye. Representative examples of fluorescent dyes include fluorescein and fluorescein dyes (e.g., fluorescein isothiocyanine (FITC), naphthofluorescein, 4′,5′-dichloro-2′,7′-dimethoxy-fluorescein, 6-carboxyfluorescein or FAM), carbocyanine, merocyanine, styryl dyes, oxonol dyes, phycoerythrin, erythrosin, eosin, rhodamine dyes (e.g., 5-carboxytetramethylrhodamine (TAMRA), carboxyrhodamine 6G, carboxy-X-rhodamine (ROX), lissamine rhodamine B, rhodamine 6G, rhodamine Green, rhodamine Red, or tetramethylrhodamine (TMR)), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g. Cy-3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, TRD 700, IRD 800), and the like. For more examples of suitable fluorescent dyes and methods for coupling fluorescent dyes to other chemical entities see, for example, The Handbook of Fluorescent Probes and Research Products, 9th Ed., Molecular Probes, Inc., Eugene, Oregon and Molecular Probes Handbook, A Guide to Fluorescent Probes and Labeling Technologies, 11^{th} Ed., Life Technologies.

In some embodiments, the diagnostic moiety includes a rhodamine dye. In some embodiments, the diagnostic moiety includes tetramethylrhodamine (TAMRA) or a derivative thereof.

In some embodiments, the diagnostic moiety is a chromogenic agent, which as known in the art refers to a chemical compound that induces a color reaction. Representative examples of chromogenic agents include azo reagents such as methyl orange and methyl red, nitrophenols, phthaleins such as phenolphthalein or thymolphthalein, sulfonephthaleins such as bromophenol blue or bromocresol green, indophenols such as 2,6-dichlorophenolindophenol, azine reagents such as thiazine dye methylene blue, indigo carmine, derivatives of diphenylamine such as diphenylamine-4-sulfonic acid and variamine blue, arsenazo III, catechol violet, dithizone, 1-(2′-pyridylazo)-2-naphthol, 4-(2′-pyridylazo)resorcinol, chrome azurol S, eriochrome black T, eriochrome blue-black B, pyrogallol red, alizarin complexone, methylthymol blue, and xylenol orange.

In some embodiments, the diagnostic moiety is a PET tracer, which as known in the art refers to a radioligand used for imaging purposes. Representative examples include acetate (C-11), chline (C-11), fludeoxyglucose (F-18), sodium fluoride (F-18), fluoro-ethyl-spirpersone (F-18), methionine (C-11), prostate-specific membrane antigen (PSMA) (Ga-68), DOTATOC/DOTANOC/DOTATATE (Ga-68), florbetaben/florbetapir (F-18), rubidium (Rb-82), and FDDNP (F-18).

In some embodiments, the diagnostic moiety is a MRI contrast agent, which as known in the art refers to an agent that is used to improve the visibility of internal body structures.

Representative examples include gadoterate, gadodiamide, gadobenate, gadopentetate, gadoteridol, gadofosveset, gadoveresetamide, gadoxetate, and gadobutrol.

Labels suitable for use in the present invention may be detectable by any of a variety of means including spectroscopic, photochemical, biochemical, immunochemical, electrical, optical, and chemical means.

In some embodiments, the active moiety is an immobilizing moiety. Representative examples of immobilizing moieties include polystyrene beads, magnetic agarose beads, crosslinked agarose beads, and TENTAGEL® beads.

In some embodiments, R₂ is methyl, ethyl, isopropyl, or t-butyl.

In some embodiments, the compound of formula (I) is Me

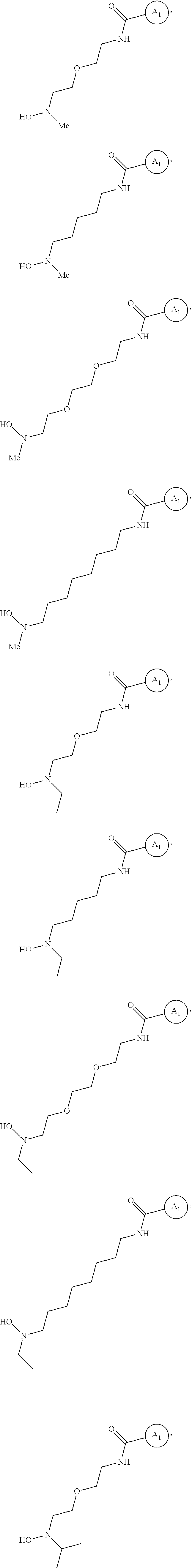

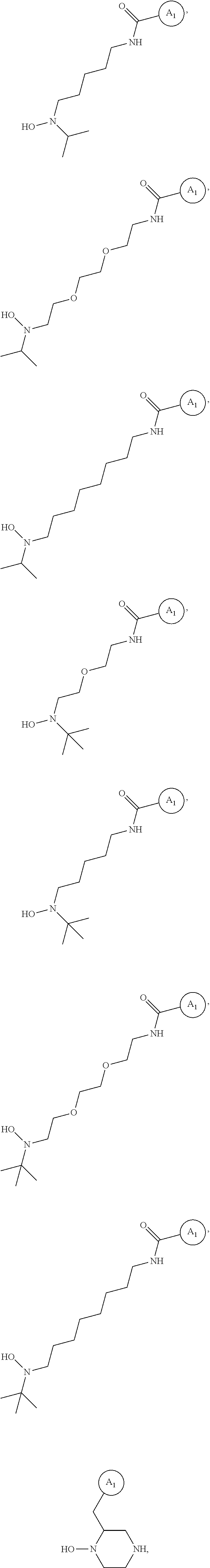

or a pharmaceutically acceptable salt or stereoisomer thereof.

In some embodiments, the optional substituent for a compound of formula (I) is selected from the group comprising of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, and azido.

In some embodiments, the compound of formula (I) is of formula Ia′, Ib, or Ic′:

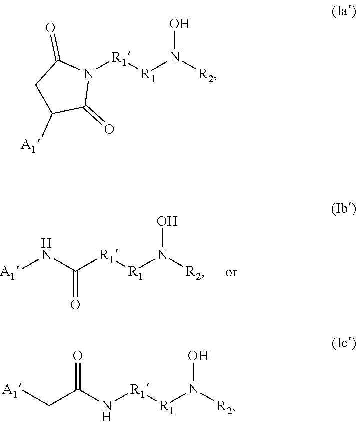

or a pharmaceutically acceptable salt or stereoisomer thereof,
wherein:
R₁′ is a linking group;
R₁ is absent, or
R₁ and R₂, together with the nitrogen atom to which they are attached, form a heterocyclyl;
R₂ is optionally substituted (C₁-C₈) alkyl, —C(O)R′, —C(O)OR′, —C(O)NR′R′, —S(O)R′, —S(O)₂R′, (C₃-C₁₀) carbocyclyl, or 4- or 7-membered heterocyclyl, wherein each R′ is independently hydrogen, (C₁-C₆) alkyl, (C₃-C₁₀) carbocyclyl, 4- or 7-membered heterocyclyl, and wherein said alkyl, carbocyclyl, or heterocyclyl is optionally substituted; and A₁′ is an antibody or an antibody fragment.

In some embodiments, R₁ is absent.

In some embodiments, R₁ is absent and R₁′ is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C1-C4 alkylene chain. In some embodiments, the alkylene chain is a C₁-C₂ alkylene chain. In some embodiments, the alkylene chain is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —C(O)—. In some embodiments, the alkylene chain is interrupted by, and/or terminates (at either or both termini) with —C(O)O—. In some embodiments, the alkylene chain is interrupted by, and/or terminates (at either or both termini) with —C(O)N(R′)—. In some embodiments, the alkylene chain is interrupted by, and/or terminates (at either or both termini) with —N(R′)S(O)₂—.

In some embodiments, R₁ is absent and R₁′ is a polyethylene glycol chain, which may be interrupted by, and/or terminate (at either or both termini) in at least one of —O—, —S—, —N(R′)—C≡C—, —C(O)—, —C(O)O—, —OC(O)—, —OC(O)O—, —C(NOR′)—, —C(O)N(R′)—, —C(O)N(R′)C(O)—R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the polyethylene glycol chain has 1 to 20 —(CH₂CH₂—O—)— units. In some embodiments, the polyethylene glycol chain has 1 to 15 —(CH₂CH₂—O—)— units. In some embodiments, the polyethylene glycol chain has 1 to 10 —(CH₂CH₂—O—)— units. In some embodiments, the polyethylene glycol chain has 1 to 5 —(CH₂CH₂—O—)— units. In some embodiments, the polyethylene glycol chain has 1 to 2 —(CH₂CH₂—O—)— units. In some embodiments, the polyethylene glycol is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —C(O)—. In some embodiments, the polyethylene glycol chain is interrupted by, and/or terminates (at either or both termini) with —C(O)O—. In some embodiments, the polyethylene glycol chain is interrupted by, and/or terminates (at either or both termini) with —C(O)N(R′)—. In some embodiments, the polyethylene glycol chain is interrupted by, and/or terminates (at either or both termini) with —N(R′)S(O)₂—.

In some embodiments, R₁ and R₂, together with the nitrogen atom to which they are attached, form a 3- to 16-membered heterocyclyl containing 1-8 heteroatoms selected from N, O, and S. In some embodiments, R₁ and R₂, together with the nitrogen atom to which they are attached, form a 4- to 12-membered heterocyclyl containing 1-4 heteroatoms selected from N, O, and S. In some embodiments, R₁ and R₂, together with the nitrogen atom to which they are attached, form a 5- to 10-membered heterocyclyl containing 1-3 heteroatoms selected from N, O, and S. In some embodiments, R₁ and R₂, together with the nitrogen atom to which they are attached, form a 5- to 6-membered heterocyclyl containing 1-2 heteroatoms selected from N, O, and S. In some embodiments, R₁ and R₂, together with the nitrogen atom to which they are attached, form a piperazinyl group.

In some embodiments, R₁ is absent, R₁′ is a C₁-C₂₄ alkylene chain and R₂ is methyl or benzyl. In some embodiments, R₁ is absent, R₁′ is a C₁-C₁₈ alkylene chain and R₂ is methyl or benzyl. In some embodiments, R₁ is absent, R₁′ is a C₁-C₁₂ alkylene chain and R₂ is methyl or benzyl. In some embodiments, R₁ is absent, R₁′ is a C₁-C₁₀ alkylene chain and R₂ is methyl or benzyl. In some embodiments, R₁ is absent, R₁′ is a C1-C₈ alkylene chain and R₂ is methyl or benzyl. In some embodiments, R₁ is absent, R₁′ is a C1-C₆ alkylene chain and R₂ is methyl or benzyl. In some embodiments, R₁ is absent, R₁′ is a C₁-C₄ alkylene chain and R₂ is methyl or benzyl. In some embodiments, R₁ is absent, R₁′ is a C₁-C₂ alkylene chain and R₂ is methyl or benzyl. In some embodiments, R₁ is absent, R₁′ is 1 to 20 —(CH₂CH₂—O—)— units and R₂ is methyl or benzyl. In some embodiments, R₁ is absent, R₁′ is 1 to 15 —(CH₂CH₂—O—)— units and R₂ is methyl or benzyl. In some embodiments, R₁ is absent, R₁′ is 1 to 10 —(CH₂CH₂—O—)— units and R₂ is methyl or benzyl. In some embodiments, R₁ is absent, R₁′ is 1 to 5 —(CH₂CH₂—O—)— units and R₂ is methyl or benzyl. In some embodiments, R₁ is absent, R₁′ is 1 to 2 —(CH₂CH₂—O—)— units and R₂ is methyl or benzyl.

In some embodiments, A₁′ is muromonab-CD3, abciximab, rituximab, palivizumab, infliximab, trastuzumab, alemtuzumab, adalimumab, ibritumomab, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab, pertuzumab, raxibacumab, obinutuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab, brodalumab, guselkumab, dupilumab, sarilumab, avelumab, ocrelizumab, emicizumab, benralizumab, gemtuzumab, durvalumab, burosumab, lanadelumab, mogamulizumab, erenumab, galcanezumab, tildrakizumab, cemiplimab, emapalumab, fremanezumab, ibalizumab, moxetumomab, ravulizumab, caplacizumab, romosozumab, risankizumab, polatuzumab, brolucizumab, crizanlizumab, sacituzumab, belantamab, or enfortumab or an antigen-binding fragment thereof. In some embodiments, A₁′ is trastuzumab.

In some embodiments, the compound of formula (Ia′) is:

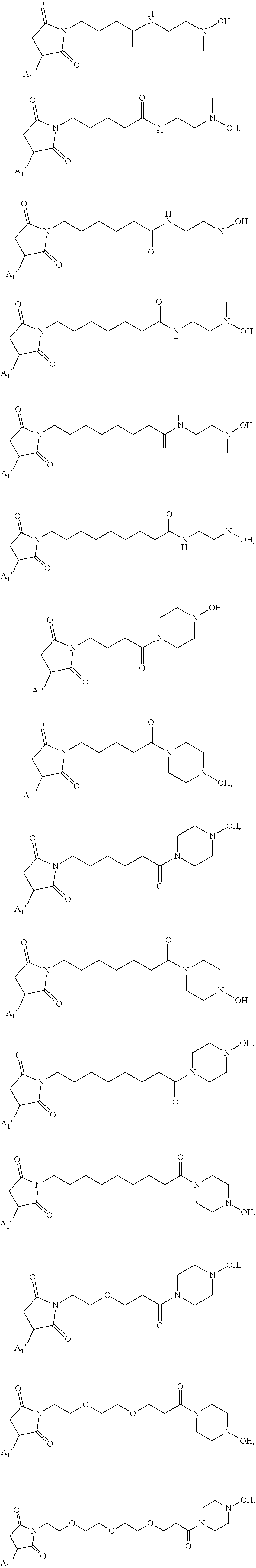

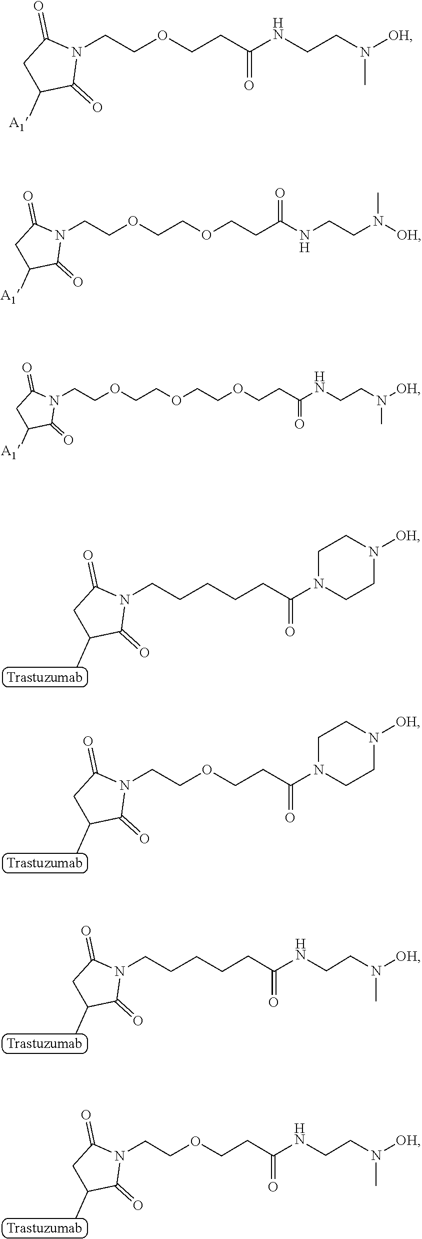

or a pharmaceutically acceptable salt or stereoisomer thereof.

In some embodiments, the compound of formula (Ib′) is:

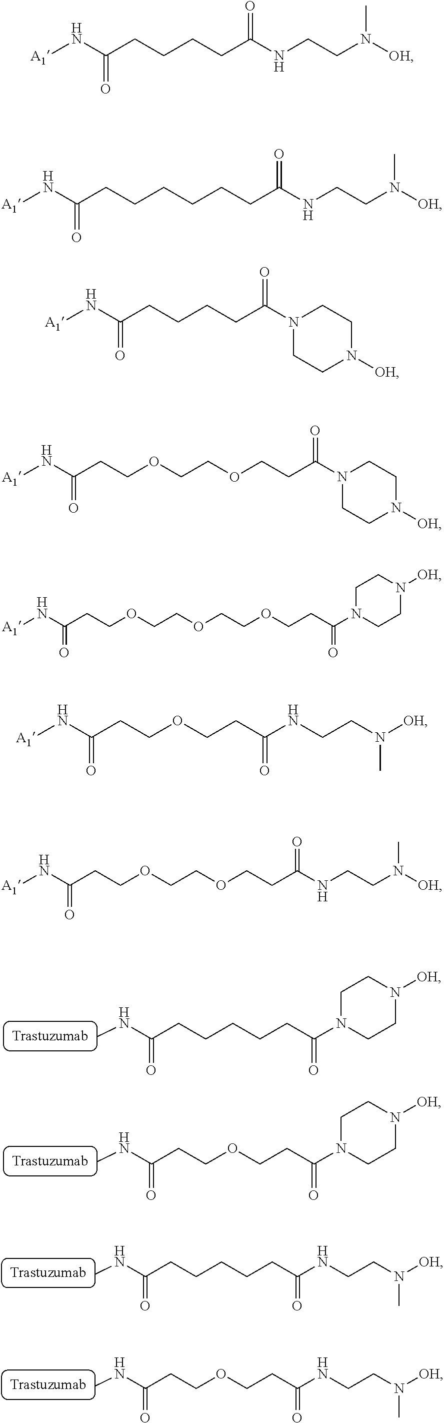

or a pharmaceutically acceptable salt or stereoisomer thereof.

In some embodiments, the compound of formula (Ic′) is:

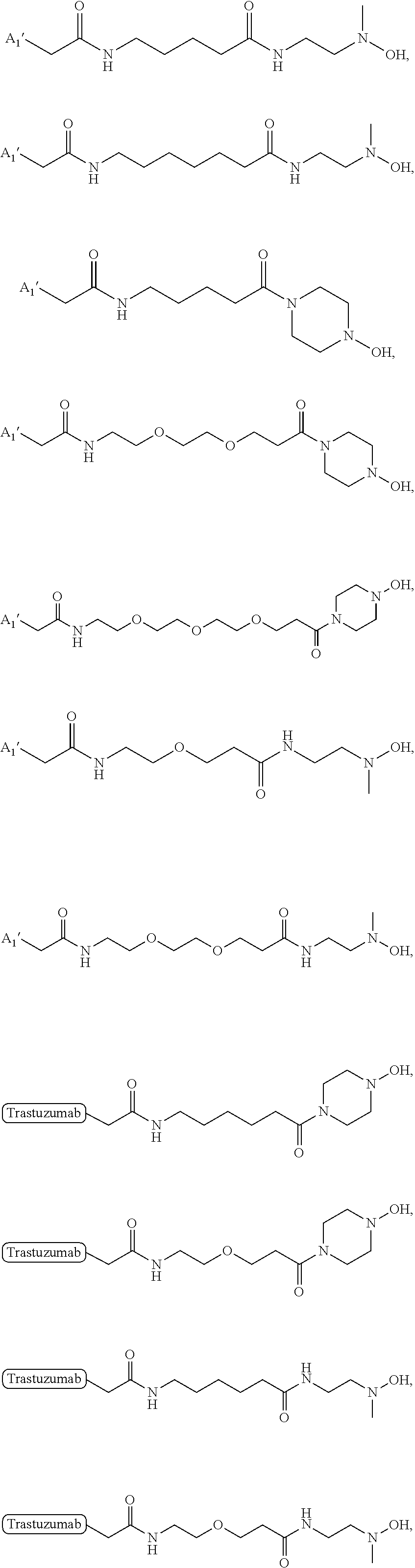

or a pharmaceutically acceptable salt or stereoisomer thereof.

Other inventive compounds of the invention are represented by formulas (II) and (III):

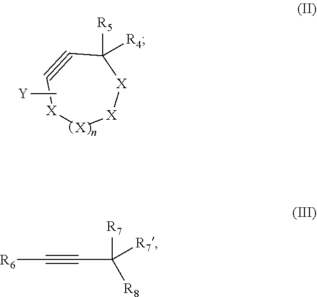

or a pharmaceutically acceptable salt or stereoisomer thereof,
wherein:
   each X is independently CR₉R₉′, NR₉, O, S, C(O), S(O), or SO₂, wherein the ring system contains 0-3 heteroatoms;
      R₉ and R₉′ are independently hydrogen or a substituent;
   Y is absent or

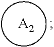

A₂ is an active moiety;
R₄ is hydrogen, a substituent or a linking group bound to an

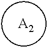

group, or
R₄ and R₅, together with the carbon atom to which they are attached, form a carbocyclyl or a heterocyclyl, wherein R₄ is also bound to an

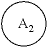

group;
R₅ is hydrogen or an electron withdrawing group;
R₆ is hydrogen, a π-electron donor group, or a linking group bound to an

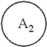

group;
R₇ and R₇′ are independently hydrogen or an electron withdrawing group, or
R₇ and R₇′, together with the carbon atom to which they are attached, form C(O);
   R₈ is hydrogen, a substituent, or a linking group bound to an

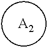

group; and
n is 1, 2, or 3,
provided that each of formulas II and III contains an

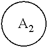

group.

In some embodiments, n is 2.

In some embodiments, X is CR₉R₉′. In some embodiments, R₉ and R₉′ are each hydrogen.

In some embodiments, R₉ and R₉′ are independently hydrogen, (C₁-C₆)alkyl, (C₁-C₆)alkoxy, (C₁-C₆)haloalkyl, (C₁-C₆)haloalkoxy, —C(O)R₁₀, —NR₁₀R₁₀, —C(O)NR₁₀R₁₀, —OC(O)NR₁₀R₁₀, —NR₁₀C(O)R₁₀, —NR₁₀C(O)OR₁₀, halogen, OH, CN, amino, (C₃-C₁₀)carbocyclyl, 4- or 7-membered heterocyclyl, —O(CH₂)₀₋₃(C₃-C₁₀)carbocyclyl, —O(CH₂)₀₋₃-4- or 7-membered heterocyclyl comprising 1 to 3 heteroatoms selected from O, N, and S, wherein each R₁₀ is independently hydrogen or (C₁-C₆) alkyl; wherein said alkyl, carbocyclyl or heterocyclyl is further optionally substituted.

In some embodiments, R₄ is a linking group bound to an

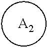

group. In some embodiments, R₄ is O. In some embodiments, R₄ is S. In some embodiments, R₄ is NR₁₁, wherein R₁₁ is hydrogen or (C₁-C₆) alkyl. In some embodiments, R₄ is OPh. In some embodiments, R₄ is OC(O). In some embodiments, R₄ is OC(O)NR₁₁, wherein R₁₁ is hydrogen or (C₁-C₆) alkyl.

In some embodiments, R₄ is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —C(O)—. In some embodiments, the alkylene chain is interrupted by, and/or terminates (at either or both termini) with —C(O)O—. In some embodiments, the alkylene chain is interrupted by, and/or terminates (at either or both termini) with —C(O)N(R′)—. In some embodiments, the alkylene chain is interrupted by, and/or terminates (at either or both termini) with —N(R′)S(O)₂—.

In some embodiments, R₄ is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the polyethylene glycol chain has 1 to 20 —(CH₂CH₂—O—)— units. In some embodiments, the polyethylene glycol chain has 1 to 15 —(CH₂CH₂—O—)— units. In some embodiments, the polyethylene glycol chain has 1 to 10 —(CH₂CH₂—O—)— units. In some embodiments, the polyethylene glycol chain has 1 to 5 —(CH₂CH₂—O—)— units. In some embodiments, the polyethylene glycol chain has 1 to 2 —(CH₂CH₂—O—)— units. In some embodiments, the polyethylene glycol is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —C(O)—. In some embodiments, the polyethylene glycol chain is interrupted by, and/or terminates (at either or both termini) with —C(O)O—. In some embodiments, the polyethylene glycol chain is interrupted by, and/or terminates (at either or both termini) with —C(O)N(R′)—. In some embodiments, the polyethylene glycol chain is interrupted by, and/or terminates (at either or both termini) with —N(R′)S(O)₂—.

In some embodiments, R₄ and R₅, together with the carbon atom to which they are attached, form a 3- to 16-membered carbocyclyl or a 3- to 16-membered heterocyclyl containing 1-8 heteroatoms selected from N, O, and S. In some embodiments, R₄ and R₅, together with the carbon atom to which they are attached, form a 4- to 12-membered carbocyclyl or 4- to 12-membered heterocyclyl containing 1-4 heteroatoms selected from N, O, and S. In some embodiments, R₄ and R₅, together with the carbon atom to which they are attached, form a 5- to 10-membered carbocyclyl or 5- to 10-membered heterocyclyl containing 1-3 heteroatoms selected from N, O, and S. In some embodiments, R₄ and R₅, together with the carbon atom to which they are attached, form a 5- to 6-membered carbocyclyl or 5- to 6-membered heterocyclyl containing 1-2 heteroatoms selected from N, O, and S.

In some embodiments, R₄ and R₅, together with the carbon atom to which they are attached, form a 5-membered heterocyclyl containing 2-oxygen atoms.

In some embodiments, R₅ is hydrogen.

In some embodiments, R₅ is an electron withdrawing group.

In some embodiments, R₅ is an inductive electron withdrawing group. In some embodiments, the inductive electron withdrawing group is halogen, OR_{5′}, SR_{5′}, or NR_{5′}R_{5′}, wherein each R_{5′} is independently hydrogen, C₁-C₆ alkyl, C₆-C₁₂ aryl, 5- to 10-membered heteroaryl, carbonyl, sulfonyl, sulfinyl, or phosphoryl.

In some embodiments, R₅ is a 7-electron withdrawing group. In some embodiments, the π-electron withdrawing group is —C(O)R_{5″}, —C(O)NR_{5″}R_{5″}, —C(O)NR_{5″}R_{5″}, —C(O)OR₅″, NO₂, CN, N₃, —S(O)R₅″, —S(O)₂R₅″, —S(O)OR₅″, —S(O)₂OR₅″, —S(O)NR_{5″}R₅″, —S(O)₂NR₅″R₅″, —OP(O)OR₅—OR₅″, —P(O)NR_{5″}R_{5″}NR_{5″}R₅″, wherein each R_{5″} is independently hydrogen, C₁-C₆ alkyl, C₆-C₁₂ aryl, 5- to 10-membered heteroaryl.

In some embodiments, R₆ is hydrogen.

In some embodiments, R₆ is a π-electron donor group.

In some embodiments, R₆ is OR₁₂, SR₁₂, NR₁₂NR₁₂, or a cyclic or acyclic amide, wherein each R₁₂ is independently hydrogen, (C₁-C₆) alkyl, (C₃-C₁₀) carbocyclyl, 4- or 7-membered heterocyclyl, wherein said alkyl, carbocyclyl, or heterocyclyl is optionally substituted.

In some embodiments, R₇ and R₇′ are independently hydrogen or an inductive electron withdrawing group. In some embodiments, the inductive electron withdrawing group is halogen, OR_{5′}, SR_{5′}, or NR_{5″}R_{5′}, wherein each R_{5′} is independently hydrogen, C₁-C₆ alkyl, C₆-C₁₂ aryl, 5 to 10-membered heteroaryl, carbonyl, sulfonyl, sulfinyl, or phosphoryl.

In some embodiments, R₇ and R₇′ are independently hydrogen or a R-electron withdrawing group. In some embodiments, the π-electron withdrawing group is —C(O)R_{5″}, —C(O)NR_{5″}R_{5″}, —C(O)NR_{5″}R_{5″}, —C(O)OR_{5″}, NO₂, CN, N₃, —S(O)R_{5″}, —S(O)₂R₅″, —S(O)OR_{5″}, —S(O)₂OR₅″, —S(O)NR_{5″}R_{5″}, —S(O)₂NR_{5″}R_{5″}, —OP(O)OR_{5″}OR_{5″}, —P(O)NR_{5″}R_{5″}NR_{5″}R_{5″}, wherein each R_{5″} is independently hydrogen, C₁-C₆ alkyl, C₆-C₁₂ aryl, 5- to 10-membered heteroaryl.

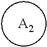

In some embodiments, R₈ is a linking group bound to an group. In some embodiments, R₈ is CH₂. In some embodiments, R₈ is C₆-C₁₂ aryl or 5- to 10-membered heteroaryl. In some embodiments, R₈ is O.

In some embodiments, R₈ is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —C(O)—. In some embodiments, the alkylene chain is interrupted by, and/or terminates (at either or both termini) with —C(O)O—. In some embodiments, the alkylene chain is interrupted by, and/or terminates (at either or both termini) with —C(O)N(R′)—. In some embodiments, the alkylene chain is interrupted by, and/or terminates (at either or both termini) with —N(R′)S(O)₂—.

In some embodiments, R₈ is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the polyethylene glycol chain has 1 to 20 —(CH₂CH₂—O—)— units. In some embodiments, the polyethylene glycol chain has 1 to 15 —(CH₂CH₂—O—)— units. In some embodiments, the polyethylene glycol chain has 1 to 10 —(CH₂CH₂—O)— units. In some embodiments, the polyethylene glycol chain has 1 to 5 —(CH₂CH₂—O)— units. In some embodiments, the polyethylene glycol chain has 1 to 2 —(CH₂CH₂—O—)— units. In some embodiments, the polyethylene glycol is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —C(O)—. In some embodiments, the polyethylene glycol chain is interrupted by, and/or terminates (at either or both termini) with —C(O)O—. In some embodiments, the polyethylene glycol chain is interrupted by, and/or terminates (at either or both termini) with —C(O)N(R′)—. In some embodiments, the polyethylene glycol chain is interrupted by, and/or terminates (at either or both termini) with —N(R′)S(O)₂—.

The A₂ moiety is an active moiety defined identically as for A₁, above in connection with compounds of formula (I).

In some embodiments, the compound of formula (II) is represented by a compound of formula (IIa):

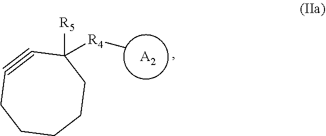

(IIa), or a pharmaceutically acceptable salt or stereoisomer thereof. In some embodiments, R₄ is O, S, NR₁₁, OPh, OC(O), OC(O)NR₁₁, wherein R₁₁ is hydrogen or (C₁-C₆) alkyl, an optionally substituted alkylene chain, or an optionally substituted polyethylene glycol chain; and/or R₅ is hydrogen, fluoro, or OR_{5′}, wherein OR_{5′} is hydrogen or (C₁-C₆) alkyl; and/or A₂ is a binding moiety, a therapeutic moiety or a diagnostic moiety.

In some embodiments, the compound of formula (II) is

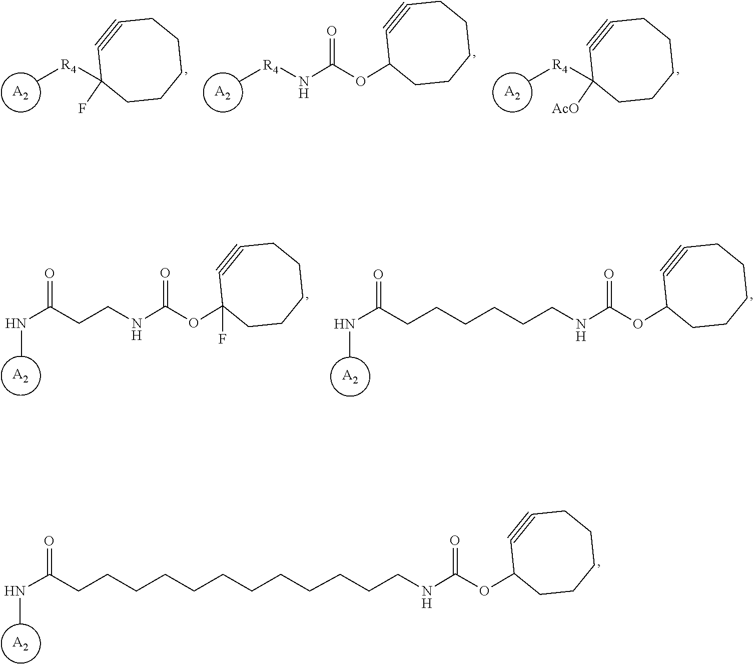

or a pharmaceutically acceptable salt or stereoisomer thereof.

In some embodiments, the compound of formula (II′) is represented by a compound of formula (II′):

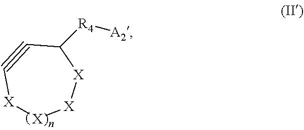

or a pharmaceutically acceptable salt or stereoisomer thereof,
wherein:
each X is independently CR₉R₉′, NR₉, O, S, C(O), S(O), or SO₂, wherein the ring system contains 0-3 heteroatoms;
   R₉ and R₉′ are independently hydrogen or a substituent;
R₄ is a linking group;
A₂′ is a therapeutic small molecule; and
n is 1, 2, or 3.

In some embodiments, X is CR₉R₉′. In some embodiments, R₉ and R₉′ are each hydrogen. In some embodiments, R₉ and R₉′ are independently hydrogen, (C₁-C₆)alkyl, (C₁-C₆)alkoxy, (C₁-C₆)haloalkyl, (C₁-C₆)haloalkoxy, —C(O)R₁₀, —NR₁₀R₁₀, —C(O)NR₁₀R₁₀, —OC(O)NR₁₀R₁₀, —NR₁₀C(O)R₁₀, —NR₁₀C(O)OR₁₀, halogen, OH, CN, amino, (C₃-C₁₀)carbocyclyl, 4- or 7-membered heterocyclyl, —O(CH₂)₀₋₃(C₃-C₁₀)carbocyclyl, —O(CH₂)₀₋₃-4- or 7-membered heterocyclyl comprising 1 to 3 heteroatoms selected from O, N, and S, wherein each R₁₀ is independently hydrogen or (C₁-C₆) alkyl; wherein the alkyl, carbocyclyl or heterocyclyl is further optionally substituted.

In some embodiments, R₄ is O, S, NR₁₀, OC(O), NR₁₀C(O), or OC(O)NR₅, wherein R₁₀ is hydrogen or C₁-C₆ alkyl.

In some embodiments, R₄ is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —C(O)—. In some embodiments, the alkylene chain is interrupted by, and/or terminates (at either or both termini) with —C(O)O—. In some embodiments, the alkylene chain is interrupted by, and/or terminates (at either or both termini) with —C(O)N(R′)—. In some embodiments, the alkylene chain is interrupted by, and/or terminates (at either or both termini) with —N(R′)S(O)₂—.

In some embodiments, R₄ is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the polyethylene glycol chain has 1 to 20 —(CH₂CH₂—O)— units. In some embodiments, the polyethylene glycol chain has 1 to 15 —(CH₂CH₂—O—)— units. In some embodiments, the polyethylene glycol chain has 1 to 10 —(CH₂CH₂—O—)— units. In some embodiments, the polyethylene glycol chain has 1 to 5 —(CH₂CH₂—O)— units. In some embodiments, the polyethylene glycol chain has 1 to 2 —(CH₂CH₂—O)— units. In some embodiments, the polyethylene glycol is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —C(O)—. In some embodiments, the polyethylene glycol chain is interrupted by, and/or terminates (at either or both termini) with —C(O)O—. In some embodiments, the polyethylene glycol chain is interrupted by, and/or terminates (at either or both termini) with —C(O)N(R′)—. In some embodiments, the polyethylene glycol chain is interrupted by, and/or terminates (at either or both termini) with —N(R′)S(O)₂—.

In some embodiments, n is 2. In some embodiments, n is 2 and each X is CH₂, and the structure represented by formula II′a:

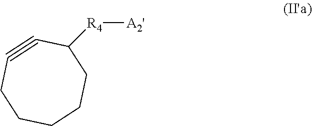

or a pharmaceutically acceptable salt or stereoisomer thereof.

In some embodiments, A₂′ is an anti-cancer agent. In some embodiments, A₂′ is an auristatin, a maytansinoid, a tubulysin, an anthracycline, paclitaxel or docetaxel or derivative thereof, calicheamicin or a derivative thereof, pyrrolobenzodiazepine dimer (PBD) or a derivative thereof, duocarmycin or a derivative thereof, eribulin or a derivative thereof, camptothecin or a derivative thereof, or exatecan or a derivative thereof.

Representative examples of auristatins include dolastatin

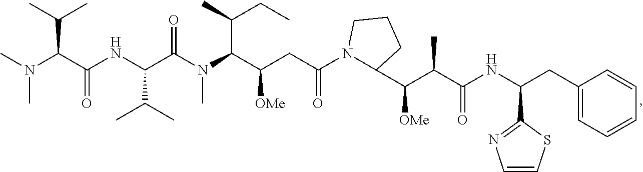 [SMILES: CC[C@H](C)[C@@H]([C@@H](CC(=O)N1CCCC1[C@H](OC)[C@@H](C)C(=O)N[C@@H](Cc1ccccc1)c1nccs1)OC)N(C)C(=O)[C@@H](NC(=O)[C@H](C(C)C)N(C)C)C(C)C]

monomethyl auristatin E (MMAE)

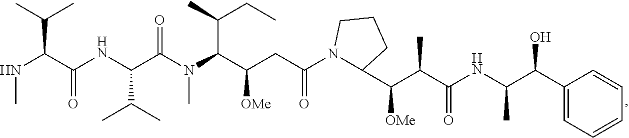 [SMILES: CC[C@H](C)[C@@H]([C@@H](CC(=O)N1CCCC1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

monomethyl auristatin F (MMAF)

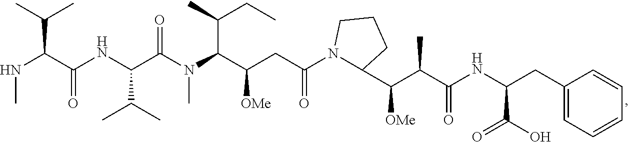 [SMILES: CC[C@H](C)[C@@H]([C@@H](CC(=O)N1CCCC1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

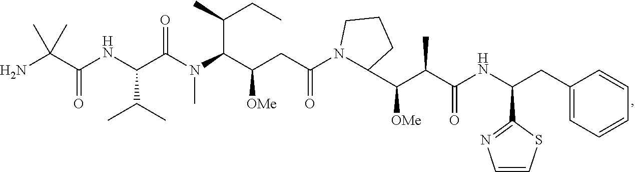 [SMILES: CC[C@H](C)[C@@H]([C@@H](CC(=O)N1CCCC1[C@H](OC)[C@@H](C)C(=O)N[C@@H](Cc1ccccc1)c1nccs1)OC)N(C)C(=O)[C@@H](NC(=O)C(C)(C)N)C(C)C]

and azastatin-OMe

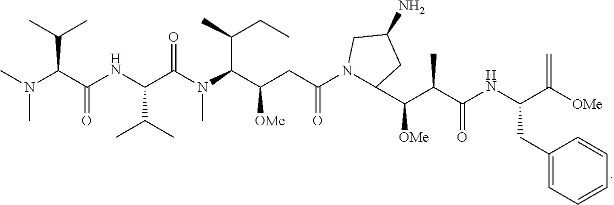 [SMILES: C=C(OC)[C@H](Cc1ccccc1)NC(=O)[C@H](C)[C@@H](OC)C1C[C@H](N)CN1C(=O)C[C@@H](OC)[C@H]([C@@H](C)CC)N(C)C(=O)[C@@H](NC(=O)[C@H](C(C)C)N(C)C)C(C)C]

Representative examples of maytansinoids include maytansine

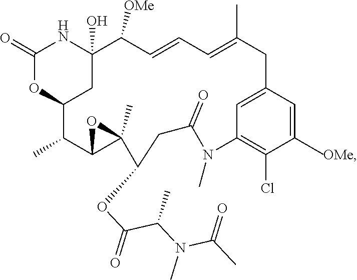 [SMILES: CO[C@@H]1/C=C/C=C(\C)Cc2cc(C)c(Cl)c(c2)N(C)C(=O)C[C@H](OC(=O)[C@H](C)N(C)C(C)=O)[C@]2(C)O[C@H]2[C@H](C)[C@@H]2C[C@@]1(O)NC(=O)O2]

### DM1

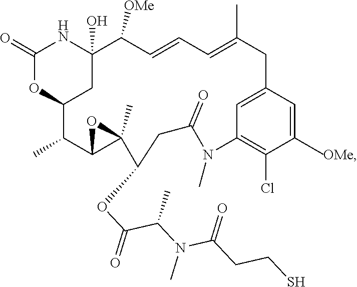 [SMILES: CO[C@@H]1/C=C/C=C(\C)Cc2cc(C)c(Cl)c(c2)N(C)C(=O)C[C@H](OC(=O)[C@H](C)N(C)C(=O)CCS)[C@]2(C)O[C@H]2[C@H](C)[C@@H]2C[C@@]1(O)NC(=O)O2]

### DM3

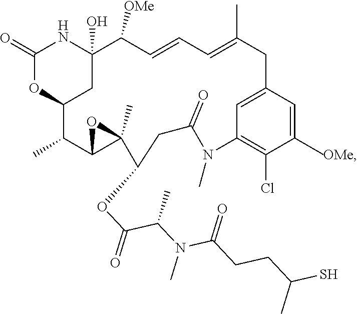 [SMILES: CO[C@@H]1/C=C/C=C(\C)Cc2cc(C)c(Cl)c(c2)N(C)C(=O)C[C@H](OC(=O)[C@H](C)N(C)C(=O)CCC(C)S)[C@]2(C)O[C@H]2[C@H](C)[C@@H]2C[C@@]1(O)NC(=O)O2]

### and DM4

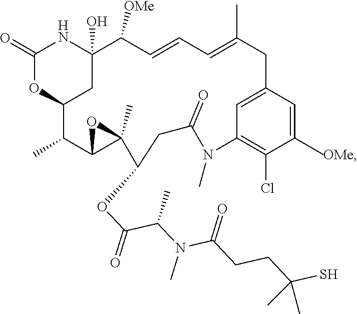 [SMILES: CO[C@@H]1/C=C/C=C(\C)Cc2cc(C)c(Cl)c(c2)N(C)C(=O)C[C@H](OC(=O)[C@H](C)N(C)C(=O)CCC(C)(C)S)[C@]2(C)O[C@H]2[C@H](C)[C@@H]2C[C@@]1(O)NC(=O)O2]

Representative examples of tubulysins include tubulysin A

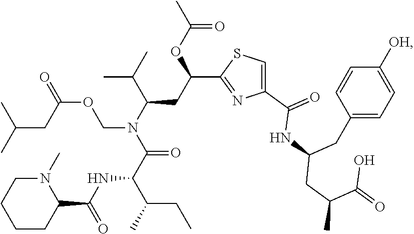

tubulysin B

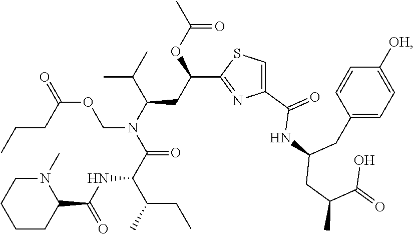

tubulysin C

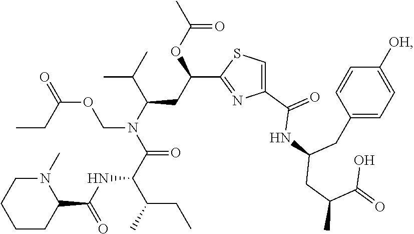

tubulysin G

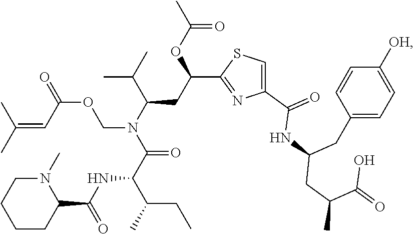

tubulysin I

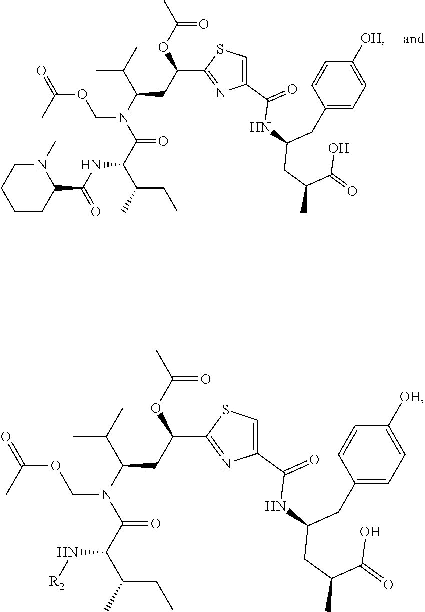

and wherein
R₁ is CH₂CH(CH₃)₂, CH₂CH₂CH₃, CH₂CH₃, CH═C(CH₃)₂, or CH₃; and
R₂ is

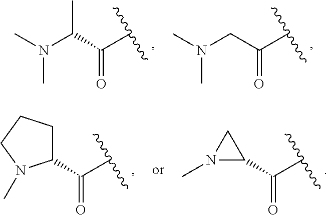

Representative examples of anthracyclines include doxorubicin

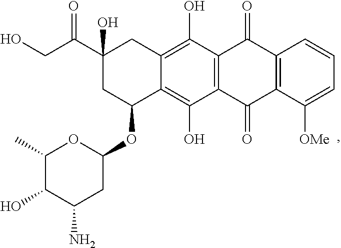 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

### PNU-159682

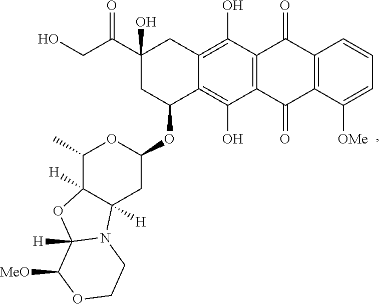 [SMILES: COc1cccc2c1C(=O)c1c(O)c3c(c(O)c1C2=O)C[C@@](O)(C(=O)CO)C[C@@H]3O[C@H]1C[C@@H]2[C@@H](O[C@H]3[C@H](OC)OCCN23)[C@H](C)O1]

and ladirubicin

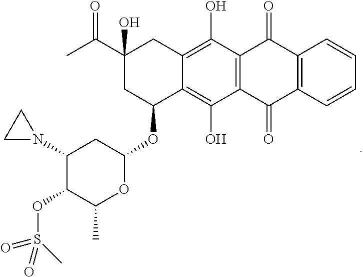 [SMILES: CC(=O)[C@]1(O)Cc2c(O)c3c(c(O)c2[C@@H](O[C@H]2C[C@@H](N4CC4)[C@@H](OS(C)(=O)=O)[C@@H](C)O2)C1)C(=O)c1ccccc1C3=O]

Suitable sites for conjugation on the anti-cancer agents, e.g., as described above, are readily identified by persons skilled in the art and are otherwise described in the literature. See, Kostova et al., Pharmaceuticals, 14:442 (2021).

In some embodiments, the compound of formula (IIa′) is:

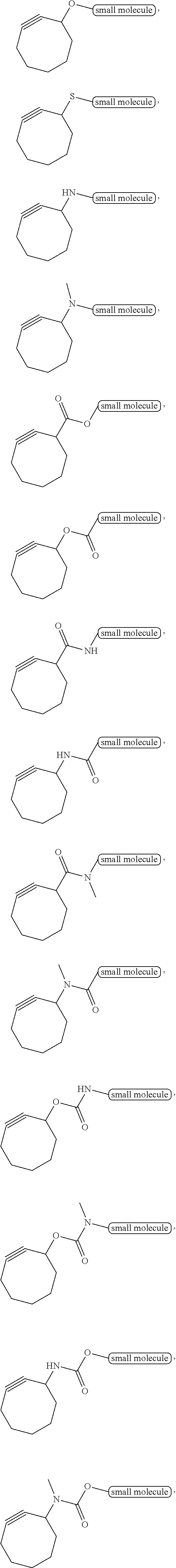

or a pharmaceutically acceptable salt or stereoisomer thereof.

In some embodiments, the compound of formula (III) is represented by a compound of formula (IIIa):

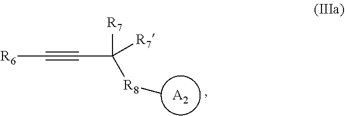

or a pharmaceutically acceptable salt or stereoisomer thereof. In some embodiments, R₆ is hydrogen, chloro, bromo, iodo, OR₁₂, or SR₁₂, wherein each R₁₂ is independently hydrogen, (C₁-C₆) alkyl, (C₃-C₁₀) carbocyclyl, 4- or 7-membered heterocyclyl; and/or R₇ is hydrogen, fluoro, or OR_{5′}, wherein R_{5′} is hydrogen or (C₁-C₆) alkyl; and/or R_{7′} is hydrogen, fluoro, or OR_{5′}, wherein R_{5′} is hydrogen or (C₁-C₆) alkyl; and R₈ is CH₂, O, C₆-C₁₂ aryl, 5- to 10-membered heteroaryl, an optionally substituted alkylene chain, or an optionally substituted polyethylene glycol chain; and/or A₂ is a binding moiety, a therapeutic moiety or a diagnostic moiety.

In some embodiments, the compound of formula (III) is

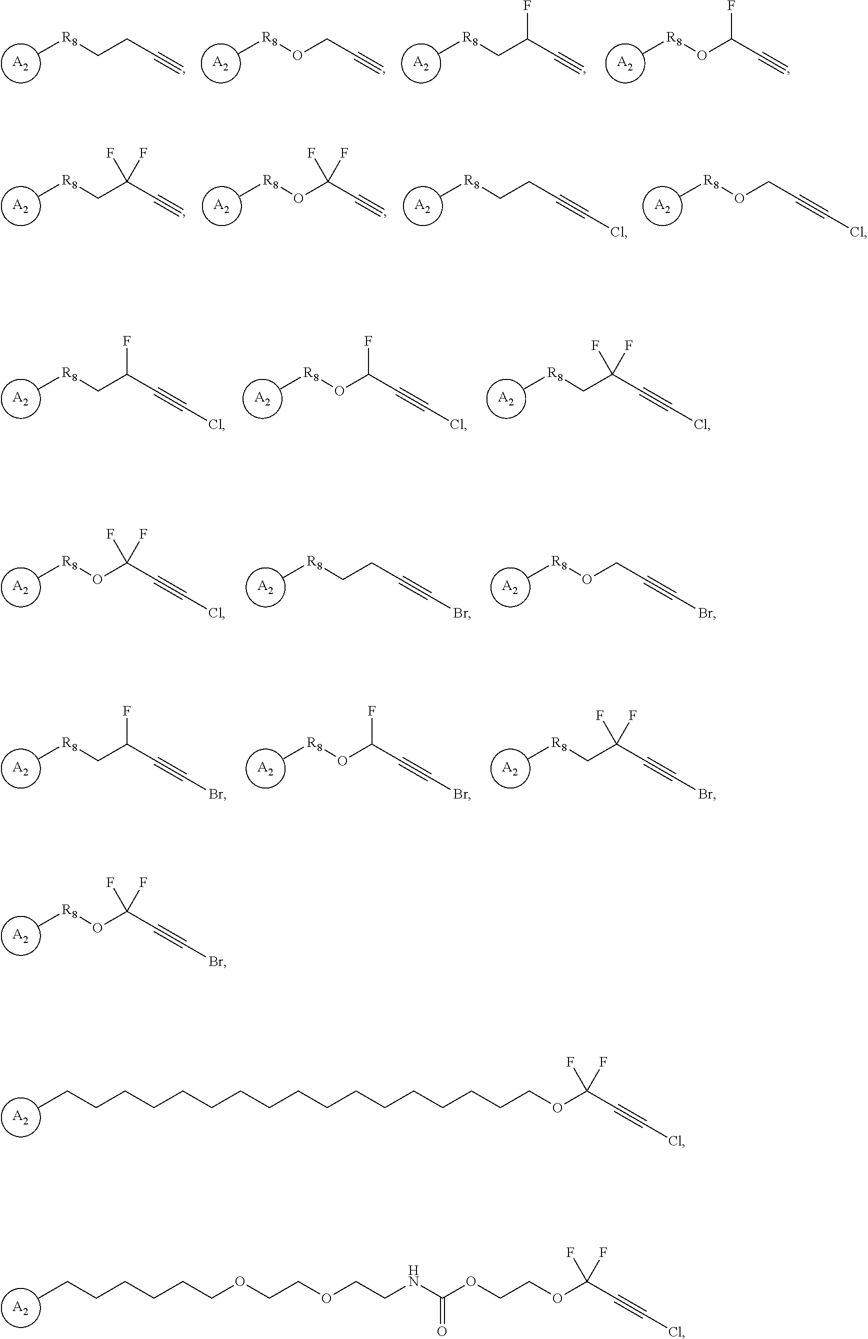

or a pharmaceutically acceptable salt or stereoisomer thereof.

In some embodiments, the optionally substituent for a compound of formula (II) or (III) is selected from the group comprising of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, and azido.

Yet other inventive compounds are represented by formulas (IV) and (V):

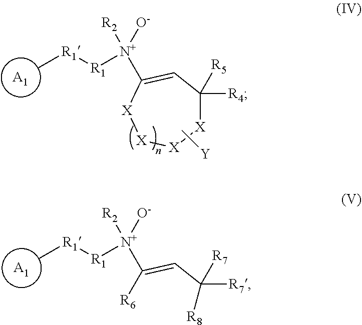

or a pharmaceutically acceptable salt or stereoisomer thereof,
wherein:
   R₁′ is a linking group;
   R₁ is absent, or
   R₁ and R₂, together with the nitrogen atom to which they are attached, form a heterocyclyl;
   R₂ is optionally substituted (C₁-C₅) alkyl, —C(O)R″, —C(O)OR″, —C(O)NR″R″, —S(O)R″, —S(O)₂R″, (C₃-C₁₀) carbocyclyl, 4- or 7-membered heterocyclyl, or a substituted polyethylene glycol chain, wherein each R″ is independently hydrogen, (C₁-C₆) alkyl, (C₃-C₁₀) carbocyclyl, 4- or 7-membered heterocyclyl, and wherein said alkyl, carbocyclyl, or heterocyclyl is optionally substituted;
   each X is independently CR₉R₉′, NR₉, O, S, C(O), S(O), or SO₂, wherein the ring system contains 0-3 heteroatoms;
      R₉ and R₉′ are independently hydrogen or a substituent;
   A₁ is an active moiety as defined above in connection with compounds of formula (I);
   Y is absent or

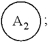

A₂ is an active moiety as defined above with respect to A₁;
R₄ is hydrogen, a substituent or a linking group bound to an

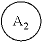

group, or
R₄ and R₅, together with the carbon atom to which they are attached, form a carbocyclyl or a heterocyclyl, wherein R₄ is also bound to an

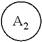

group;
R₅ is hydrogen or an electron withdrawing group;
R₆ is hydrogen, a π-electron donor group, or a linking group bound to an

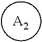

group;
R₇ and R₇′ are independently hydrogen or an electron withdrawing group, or
R₇ and R₇′, together with the carbon atom to which they are attached, form C(O);
R₈ is hydrogen, a substituent, or a linking group bound to an

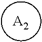

group; and
n is 1, 2, or 3;
provided that each of compounds of formulas (IV) and (V) contain at least one

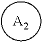

group.

In some embodiments, R₁ is absent and R₁′ is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, 4- to 6-membered heterocyclyl, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —C(O)—. In some embodiments, the alkylene chain is interrupted by, and/or terminates (at either or both termini) with —C(O)O—. In some embodiments, the alkylene chain is interrupted by, and/or terminates (at either or both termini) with —C(O)N(R′)—. In some embodiments, the alkylene chain is interrupted by, and/or terminates (at either or both termini) with —N(R′)S(O)₂—. In some embodiments, the alkylene chain is interrupted by, and/or terminates (at either or both termini) with a 4- to 6-membered heterocyclyl.

In some embodiments, the alkylene chain terminates with pyrrolidine-2,5-dione

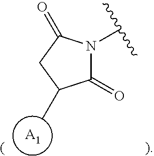

In some embodiments, R₁ is absent and R₁′, is a polyethylene glycol chain, which may be interrupted by, and/or terminate (at either or both termini) in at least one of —O—, —S—, —N(R′)—C≡C—, —C(O)—, —C(O)O—, —OC(O)—, —OC(O)O—, —C(NOR′)—, —C(O)N(R′)—, —C(O)N(R′)C(O)—R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the polyethylene glycol chain has 1 to 20 —(CH₂CH₂—O—)— units. In some embodiments, the polyethylene glycol chain has 1 to 15 —(CH₂CH₂—O—)— units. In some embodiments, the polyethylene glycol chain has 1 to 10 —(CH₂CH₂—O—)— units. In some embodiments, the polyethylene glycol chain has 1 to 5 —(CH₂CH₂—O—)— units. In some embodiments, the polyethylene glycol chain has 1 to 2 —(CH₂CH₂—O—)— units. In some embodiments, the polyethylene glycol is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, 4- to 6-membered heterocyclyl, or a combination thereof. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —C(O)—. In some embodiments, the polyethylene glycol chain is interrupted by, and/or terminates (at either or both termini) with —C(O)O—. In some embodiments, the polyethylene glycol chain is interrupted by, and/or terminates (at either or both termini) with —C(O)N(R′)—. In some embodiments, the polyethylene glycol chain is interrupted by, and/or terminates (at either or both termini) with —N(R′)S(O)₂—. In some embodiments, the polyethylene glycol chain is interrupted by, and/or terminates (at either or both termini) with a 4- to 6-membered heterocyclyl. In some embodiments, the polyethylene glycol chain terminates with pyrrolidine-2,5-dione

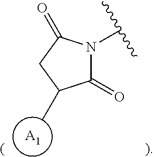

In some embodiments, R₁ and R₂, together with the nitrogen atom to which they are attached, form a 3- to 16-membered heterocyclyl containing 1-8 heteroatoms selected from N, O, and S. In some embodiments, R₁ and R₂, together with the nitrogen atom to which they are attached, form a 4- to 12-membered heterocyclyl containing 1-4 heteroatoms selected from N, O, and S. In some embodiments, R₁ and R₂, together with the nitrogen atom to which they are attached, form a 5- to 10-membered heterocyclyl containing 1-3 heteroatoms selected from N, O, and S. In some embodiments, R₁ and R₂, together with the nitrogen atom to which they are attached, form a 5- to 6-membered heterocyclyl containing 1-2 heteroatoms selected from N, O, and S.

In some embodiments, R₂ is methyl, ethyl, isopropyl, or t-butyl.

In some embodiments, the R₁ is a C₁-C₂₄ alkylene chain and R₂ is methyl, ethyl, isopropyl, or t-butyl. In some embodiments, R₁ is a C₁-C₁₈ alkylene chain and R₂ is methyl, ethyl, isopropyl, or t-butyl. In some embodiments, R₁ is a C₁-C₁₂ alkylene chain and R₂ is methyl, ethyl, isopropyl, or t-butyl. In some embodiments, R₁ is a C₁-C₁₀ alkylene chain and R₂ is methyl, ethyl, isopropyl, or t-butyl. In some embodiments, R₁ is a C₁-C₈ alkylene chain and R₂ is methyl, ethyl, isopropyl, or t-butyl. In some embodiments, R₁ is a C₁-C₆ alkylene chain and R₂ is methyl, ethyl, isopropyl, or t-butyl. In some embodiments, R₁ is a C₁-C₄ alkylene chain and R₂ is methyl, ethyl, isopropyl, or t-butyl. In some embodiments, R₁ is a C₁-C₂ alkylene chain and R₂ is methyl, ethyl, isopropyl, or t-butyl. In some embodiments R₁ is 1 to 20 —(CH₂CH₂—O—)— units and R₂ is methyl, ethyl, isopropyl, or t-butyl. In some embodiments, R₁ is 1 to 15 —(CH₂CH₂—O—)— units and R₂ is methyl, ethyl, isopropyl, or t-butyl. In some embodiments, R₁ is 1 to 10 —(CH₂CH₂—O—)— units and R₂ is methyl, ethyl, isopropyl, or t-butyl. In some embodiments, R₁ is 1 to 5 —(CH₂CH₂—O—)— units and R₂ is methyl, ethyl, isopropyl, or t-butyl. In some embodiments, R₁ is 1 to 2 —(CH₂CH₂-0)-units and R₂ is methyl, ethyl, isopropyl, or t-butyl.

In some embodiments, n is 2.

In some embodiments, X is CR₉R₉′. In some embodiments, R₉ and R₉′ are each hydrogen.

In some embodiments, R₉ and R₉′ are independently hydrogen, (C₁-C₆)alkyl, (C₁-C₆)alkoxy, (C₁-C₆)haloalkyl, (C₁-C₆)haloalkoxy, —C(O)R₁₀, —NR₁₀R₁₀, —C(O)NR₁₀R₁₀, —OC(O)NR₁₀R₁₀, —NR₁₀C(O)R₁₀, —NR₁₀C(O)OR₁₀, halogen, OH, CN, amino, (C₃-C₁₀)carbocyclyl, 4- or 7-membered heterocyclyl, —O(CH₂)₀₋₃(C₃-C₁₀)carbocyclyl, —O(CH₂)₀₋₃-4- or 7-membered heterocyclyl comprising 1 to 3 heteroatoms selected from O, N, and S, wherein each R₁₀ is independently hydrogen or (C₁-C₆) alkyl; wherein said alkyl, carbocyclyl or heterocyclyl is further optionally substituted.

In some embodiments, R₄ is a linking group bound to an

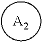

group. In some embodiments, R₄ is O. In some embodiments, R₄ is S. In some embodiments, R₄ is NR₁₁, wherein Ru is hydrogen or (C₁-C₆) alkyl. In some embodiments, R₄ is OPh. In some embodiments, R₄ is OC(O). In some embodiments, R₄ is OC(O)NR₁₁, wherein R₁ is hydrogen or (C₁-C₆) alkyl.

In some embodiments, R₄ is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂₀—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —C(O)—. In some embodiments, the alkylene chain is interrupted by, and/or terminates (at either or both termini) with —C(O)O—. In some embodiments, the alkylene chain is interrupted by, and/or terminates (at either or both termini) with —C(O)N(R′)—. In some embodiments, the alkylene chain is interrupted by, and/or terminates (at either or both termini) with —N(R′)S(O)₂—.

In some embodiments, R₄ is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the polyethylene glycol chain has 1 to 20 —(CH₂CH₂—O—)— units. In some embodiments, the polyethylene glycol chain has 1 to 15 —(CH₂CH₂—O—)— units. In some embodiments, the polyethylene glycol chain has 1 to 10 —(CH₂CH₂—O—)— units. In some embodiments, the polyethylene glycol chain has 1 to 5 —(CH₂CH₂—O—)— units. In some embodiments, the polyethylene glycol chain has 1 to 2 —(CH₂CH₂—O—)— units. In some embodiments, the polyethylene glycol is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —C(O)—. In some embodiments, the polyethylene glycol chain is interrupted by, and/or terminates (at either or both termini) with —C(O)O—. In some embodiments, the polyethylene glycol chain is interrupted by, and/or terminates (at either or both termini) with —C(O)N(R′)—. In some embodiments, the polyethylene glycol chain is interrupted by, and/or terminates (at either or both termini) with —N(R′)S(O)₂—.

In some embodiments, R₄ and R₅, together with the carbon atom to which they are attached, form a 3- to 16-membered carbocyclyl or a 3- to 16-membered heterocyclyl containing 1-8 heteroatoms selected from N, O, and S. In some embodiments, R₄ and R₅, together with the carbon atom to which they are attached, form a 4- to 12-membered carbocyclyl or 4- to 12-membered heterocyclyl containing 1-4 heteroatoms selected from N, O, and S. In some embodiments, R₄ and R₅, together with the carbon atom to which they are attached, form a 5- to 10-membered carbocyclyl or 5- to 10-membered heterocyclyl containing 1-3 heteroatoms selected from N, O, and S. In some embodiments, R₄ and R₅, together with the carbon atom to which they are attached, form a 5- to 6-membered carbocyclyl or 5- to 6-membered heterocyclyl containing 1-2 heteroatoms selected from N, O, and S.

In some embodiments, R₄ and R₅, together with the carbon atom to which they are attached, form a 5-membered heterocyclyl containing 2-oxygen atoms.

In some embodiments, R₅ is hydrogen.

In some embodiments, R₅ is an electron withdrawing group.

In some embodiments, R₅ is an inductive electron withdrawing group. In some embodiments, the inductive electron withdrawing group is halogen, OR_{5′}, SR_{5′}, or NR_{5′}R_{5′}, wherein each R_{5′} is independently hydrogen, C₁-C₆ alkyl, C₆-C₁₂ aryl, 5- to 10-membered heteroaryl, carbonyl, sulfonyl, sulfinyl, or phosphoryl.

In some embodiments, R₅ is a 7-electron withdrawing group. In some embodiments, the π-electron withdrawing group is —C(O)R_{5″}, —C(O)NR_{5″}R_{5″}, —C(O)NR_{5″}R_{5″}, —C(O)OR_{5″}, NO₂, CN, N₃, —S(O)R_{5″}, —S(O)₂R_{5″}, —S(O)OR_{5″}, —S(O)₂OR_{5″}, —S(O)NR_{5″}R_{5″}, —S(O)₂NR_{5″}R_{5″}, —OP(O)OR_{5″}OR_{5″}, —P(O)NR_{5″}R_{5″}NR_{5″}R_{5″}, wherein each R_{5″} is independently hydrogen, C₁-C₆ alkyl, C₆-C₁₂ aryl, 5- to 10-membered heteroaryl.

In some embodiments, R₆ is hydrogen.

In some embodiments, R₆ is a π-electron donor group.

In some embodiments, R₆ is OR₁₂, SR₁₂, NR₁₂NR₁₂, or a cyclic or acyclic amide, wherein each R₁₂ is independently hydrogen, (C₁-C₆) alkyl, (C₃-C₁₀) carbocyclyl, 4- or 7-membered heterocyclyl, wherein said alkyl, carbocyclyl, or heterocyclyl is optionally substituted.

In some embodiments, R₇ and R₇′ are independently hydrogen or an inductive electron withdrawing group. In some embodiments, the inductive electron withdrawing group is halogen, OR_{5′}, SR_{5′}, or NR_{5″}R_{5″}, wherein each R_{5′} is independently hydrogen, C₁-C₆ alkyl, C₆-C₁₂ aryl, 5 to 10-membered heteroaryl, carbonyl, sulfonyl, sulfinyl, or phosphoryl.

In some embodiments, R₇ and R₇′ are independently hydrogen or a R-electron withdrawing group. In some embodiments, the π-electron withdrawing group is —C(O)R_{5″}, —C(O)NR_{5″}R_{5″}, —C(O)NR_{5″}R_{5″}, —C(O)OR_{5″}, NO₂, CN, N₃, —S(O)R_{5″}, —S(O)₂R₅″, —S(O)OR_{5″}, —S(O)₂OR₅″, —S(O)NR_{5″}R_{5″}, —S(O)₂NR_{5″}R_{5″}, —OP(O)OR_{5″}OR_{5″}, —P(O)NR_{5″}R_{5″}NR_{5″}R_{5″}, wherein each R_{5″}is independently hydrogen, C₁-C₆ alkyl, C₆-C₁₂ aryl, 5- to 10-membered heteroaryl.

In some embodiments, R₈ is a linking group bound to an

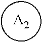

group. In some embodiments, R₈ is CH₂. In some embodiments, R₈ is aryl. In some embodiments, R₈ is O.

In some embodiments, R₈ is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —C(O)—. In some embodiments, the alkylene chain is interrupted by, and/or terminates (at either or both termini) with —C(O)O—. In some embodiments, the alkylene chain is interrupted by, and/or terminates (at either or both termini) with —C(O)N(R′)—. In some embodiments, the alkylene chain is interrupted by, and/or terminates (at either or both termini) with —N(R′)S(O)₂—.

In some embodiments, R₈ is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the polyethylene glycol chain has 1 to 20 —(CH₂CH₂—O—)— units. In some embodiments, the polyethylene glycol chain has 1 to 15 —(CH₂CH₂—O—)— units. In some embodiments, the polyethylene glycol chain has 1 to 10 —(CH₂CH₂—O—)— units. In some embodiments, the polyethylene glycol chain has 1 to 5 —(CH₂CH₂—O—)— units. In some embodiments, the polyethylene glycol chain has 1 to 2 —(CH₂CH₂—O—)— units. In some embodiments, the polyethylene glycol is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —C(O)—. In some embodiments, the polyethylene glycol chain is interrupted by, and/or terminates (at either or both termini) with —C(O)O—. In some embodiments, the polyethylene glycol chain is interrupted by, and/or terminates (at either or both termini) with —C(O)N(R′)—. In some embodiments, the polyethylene glycol chain is interrupted by, and/or terminates (at either or both termini) with —N(R′)S(O)₂—.

In some embodiments, A₁ is a therapeutic moiety and A₂ is a diagnostic moiety.

In some embodiments, A₁ is a diagnostic moiety and A₂ is a therapeutic moiety.

In some embodiments, A₁ is a therapeutic moiety and A₂ is a binding moiety.

In some embodiments, A₁ is a binding moiety and A₂ is a therapeutic moiety.

In some embodiments, A₁ is a binding moiety and A₂ is a binding moiety.

In some embodiments, the compound of formula (IV) is represented by a compound of formula (IVa):

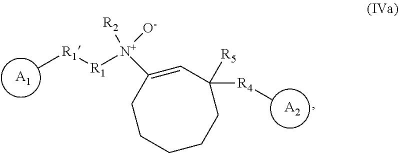

or a pharmaceutically acceptable salt or stereoisomer thereof. In some embodiments, R₁ is absent and R₁′ is an optionally substituted alkylene chain or an optionally substituted polyethylene glycol chain; and/or R₂ is methyl, ethyl, isopropyl, or t-butyl; or R₁ and R₂, together with the nitrogen atom to which they are attached, form a 3- to 16-membered heterocyclyl containing 1-8 heteroatoms selected from N, O, and S; R₁′ is CH₂; and/or R₄ is O, S, NR₁₁, OPh, OC(O), OC(O)NR₁₁, wherein R₁ is hydrogen or (C₁-C₆) alkyl, an optionally substituted alkylene chain, or an optionally substituted polyethylene glycol chain; and/or R₅ is hydrogen, fluoro, or OR_{5′}, wherein OR_{5′} is hydrogen or (C₁-C₆) alkyl; and/or A₁ is a binding moiety, a therapeutic moiety or a diagnostic moiety; and/or A₂ is a binding moiety, a therapeutic moiety or a diagnostic moiety.

In some embodiments, the compound of formula (IV) is

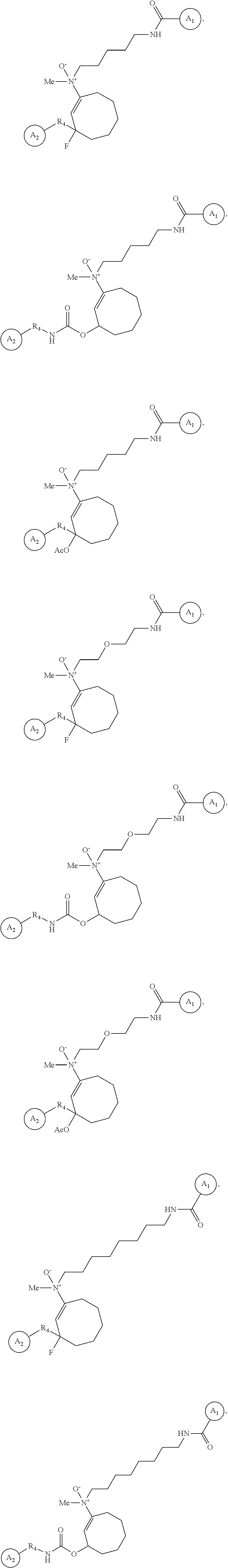

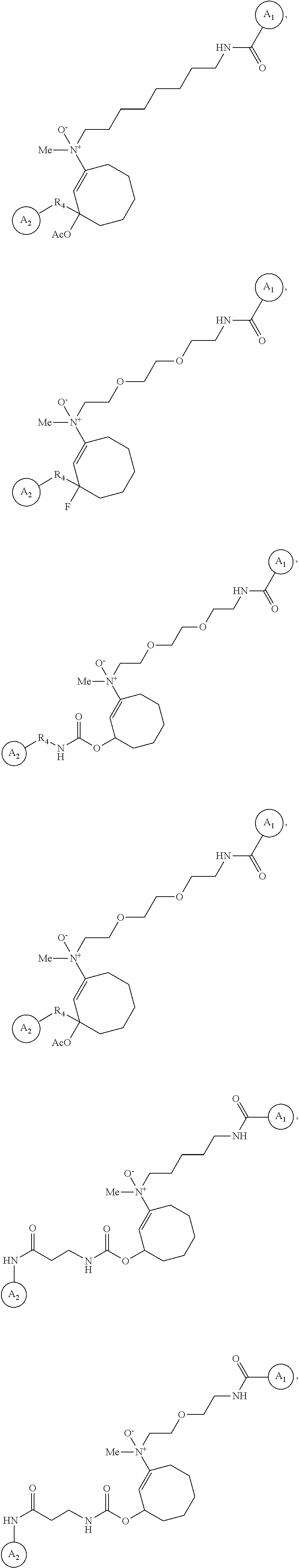

or a pharmaceutically acceptable salt or stereoisomer thereof.

In some embodiments, the compound of formula (IV) is

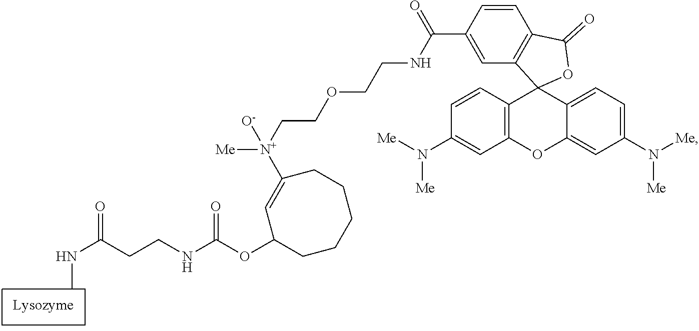 [SMILES: CNC(=O)CCNC(=O)OC1/C=C(/[N+](C)([O-])CCOCCNC(=O)c2ccc3c(c2)C2(OC3=O)c3ccc(N(C)C)cc3Oc3cc(N(C)C)ccc32)CCCCC1]

or a pharmaceutically acceptable salt or stereoisomer thereof.

In some embodiments, the compound of formula (IV) is represented by a compound of formula IVa′, IVb′, or IVc′:

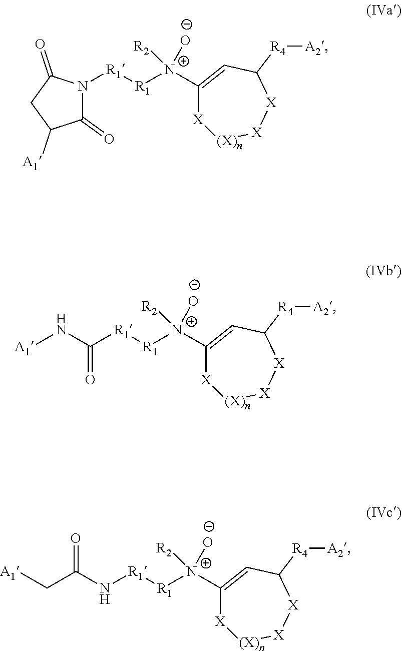

or a pharmaceutically acceptable salt or stereoisomer thereof,
wherein:
   R₁′ is a linking group;
   R₁ is absent, or
R₁ and R₂, together with the nitrogen atom to which they are attached, form a heterocyclyl;
   R₂ is optionally substituted (C₁-C₈) alkyl, —C(O)R′, —C(O)OR′, —C(O)NR′R′, —S(O)R′, —S(O)₂R′, (C₃-C₁₀) carbocyclyl, or 4- or 7-membered heterocyclyl, wherein each R′ is independently hydrogen, (C₁-C₆) alkyl, (C₃-C₁₀) carbocyclyl, 4- or 7-membered heterocyclyl, and wherein said alkyl, carbocyclyl, or heterocyclyl is optionally substituted;
A₁′ is an antibody or an antibody fragment;
each X is independently CR₉R₉′, NR₉, O, S, C(O), S(O), or SO₂, wherein the ring system contains 0-3 heteroatoms;
   R₉ and R₉′ are independently hydrogen or a substituent;
R₄ is a linking group;
A₂′ is a therapeutic small molecule; and
n is 1, 2, or 3.

In some embodiments, R₁ is absent and R₁′ is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂₀—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —C(O)—. In some embodiments, the alkylene chain is interrupted by, and/or terminates (at either or both termini) with —C(O)O—. In some embodiments, the alkylene chain is interrupted by, and/or terminates (at either or both termini) with —C(O)N(R′)—. In some embodiments, the alkylene chain is interrupted by, and/or terminates (at either or both termini) with —N(R′)S(O)₂—.

In some embodiments, R₁ is absent and R₁′ is a polyethylene glycol chain, which may be interrupted by, and/or terminate (at either or both termini) in at least one of —O—, —S—, —N(R′)—C≡C—, —C(O)—, —C(O)O—, —OC(O)—, —OC(O)O—, —C(NOR′)—, —C(O)N(R′)—, —C(O)N(R′)C(O)—R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the polyethylene glycol chain has 1 to 20 —(CH₂CH₂—O—)— units. In some embodiments, the polyethylene glycol chain has 1 to 15 —(CH₂CH₂—O—)— units. In some embodiments, the polyethylene glycol chain has 1 to 10 —(CH₂CH₂—O—)— units. In some embodiments, the polyethylene glycol chain has 1 to 5 —(CH₂CH₂—O—)— units. In some embodiments, the polyethylene glycol chain has 1 to 2 —(CH₂CH₂—O—)— units. In some embodiments, the polyethylene glycol is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —C(O)—. In some embodiments, the polyethylene glycol chain is interrupted by, and/or terminates (at either or both termini) with —C(O)O—. In some embodiments, the polyethylene glycol chain is interrupted by, and/or terminates (at either or both termini) with —C(O)N(R′)—. In some embodiments, the polyethylene glycol chain is interrupted by, and/or terminates (at either or both termini) with —N(R′)S(O)₂—.

In some embodiments, R₁ and R₂, together with the nitrogen atom to which they are attached, form a 3- to 16-membered heterocyclyl containing 1-8 heteroatoms selected from N, O, and S; and R₁′ is a C₁-C₂₄ alkylene chain or 1 to 20 —(CH₂CH₂—O—)— units, wherein R₁′ is optionally substituted. In some embodiments, R₁ and R₂, together with the nitrogen atom to which they are attached, form a 4- to 12-membered heterocyclyl containing 1-4 heteroatoms selected from N, O, and S; and R₁′ is a C₁-C₁₈ alkylene chain or 1 to 15 —(CH₂CH₂—O—)— units, wherein R₁′ is optionally substituted. In some embodiments, R₁ and R₂, together with the nitrogen atom to which they are attached, form a 5- to 10-membered heterocyclyl containing 1-3 heteroatoms selected from N, O, and S; and R₁′ is a C₁-C₁₂ alkylene chain or 1 to 10 —(CH₂CH₂—O—)— units, wherein R₁′ is optionally substituted. In some embodiments, R₁ and R₂, together with the nitrogen atom to which they are attached, form a 5- to 6-membered heterocyclyl containing 1-2 heteroatoms selected from N, O, and S; and R₁′ is a C₁-C₁₀ alkylene chain or 1 to 5 —(CH₂CH₂—O—)— units, wherein R₁′ is optionally substituted. In some embodiments, R₁ and R₂, together with the nitrogen atom to which they are attached, form a piperazinyl group; and R₁′ is a C₁-C₁₀ alkylene chain or 1 to 5 —(CH₂CH₂-0)-units, wherein R₁′ is optionally substituted.

In some embodiments, R₁ is absent, R₁′ is a C₁-C₂₄ alkylene chain and R₂ is methyl or benzyl. In some embodiments, R₁ is absent, R₁′ is a C₁-C₁₈ alkylene chain and R₂ is methyl or benzyl. In some embodiments, R₁ is absent, R₁′ is a C₁-C₁₂ alkylene chain and R₂ is methyl or benzyl. In some embodiments, R₁ is absent, R₁′ is a C₁-C₁₀ alkylene chain and R₂ is methyl or benzyl. In some embodiments, R₁ is absent, R₁′ is a C₁-C₈ alkylene chain and R₂ is methyl or benzyl. In some embodiments, R₁ is absent, R₁′ is a C₁-C₆ alkylene chain and R₂ is methyl or benzyl. In some embodiments, R₁ is absent, R₁′ is a C₁-C₄ alkylene chain and R₂ is methyl or benzyl. In some embodiments, R₁ is absent, R₁′ is a C₁-C₂ alkylene chain and R₂ is methyl or benzyl. In some embodiments, R₁ is absent, R₁′ is 1 to 20 —(CH₂CH₂—O—)— units and R₂ is methyl or benzyl. In some embodiments, R₁ is absent, R₁′ is 1 to 15 —(CH₂CH₂—O—)— units and R₂ is methyl or benzyl. In some embodiments, R₁ is absent, R₁′ is 1 to 10 —(CH₂CH₂—O—)— units and R₂ is methyl or benzyl. In some embodiments, R₁ is absent, R₁′ is 1 to 5 —(CH₂CH₂—O—)— units and R₂ is methyl or benzyl. In some embodiments, R₁ is absent, R₁′ is 1 to 2 —(CH₂CH₂—O—)— units and R₂ is methyl or benzyl.

In some embodiments, A₁′ is muromonab-CD3, abciximab, rituximab, palivizumab, infliximab, trastuzumab, alemtuzumab, adalimumab, ibritumomab, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab, pertuzumab, raxibacumab, obinutuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab, brodalumab, guselkumab, dupilumab, sarilumab, avelumab, ocrelizumab, emicizumab, benralizumab, gemtuzumab, durvalumab, burosumab, lanadelumab, mogamulizumab, erenumab, galcanezumab, tildrakizumab, cemiplimab, emapalumab, fremanezumab, ibalizumab, moxetumomab, ravulizumab, caplacizumab, romosozumab, risankizumab, polatuzumab, brolucizumab, crizanlizumab, sacituzumab, belantamab, or enfortumab or an antigen-binding fragment thereof. In some embodiments, A₁′ is trastuzumab.

In some embodiments, X is CR₉R₉′. In some embodiments, R₉ and R₉′ are each hydrogen. In some embodiments, R₉ and R₉′ are independently hydrogen, (C₁-C₆)alkyl, (C₁-C₆)alkoxy, (C₁-C₆)haloalkyl, (C₁-C₆)haloalkoxy, —C(O)R₁₀, —NR₁₀R₁₀, —C(O)NR₁₀R₁₀, —OC(O)NR₁₀R₁₀, —NR₁₀C(O)R₁₀, —NR₁₀C(O)OR₁₀, halogen, OH, CN, amino, (C₃-C₁₀)carbocyclyl, 4- or 7-membered heterocyclyl, —O(CH₂)₀₋₃(C₃-C₁₀)carbocyclyl, —O(CH₂)₀₋₃-4- or 7-membered heterocyclyl comprising 1 to 3 heteroatoms selected from O, N, and S, wherein each R₁₀ is independently hydrogen or (C₁-C₆) alkyl; wherein said alkyl, carbocyclyl or heterocyclyl is further optionally substituted.

In some embodiments, R₄ is O, S, NR₁₀, OC(O), NR₁₀C(O), or OC(O)NR₅, wherein R₁₀ is hydrogen or C₁-C₆ alkyl.

In some embodiments, R₄ is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —C(O)—. In some embodiments, the alkylene chain is interrupted by, and/or terminates (at either or both termini) with —C(O)O—. In some embodiments, the alkylene chain is interrupted by, and/or terminates (at either or both termini) with —C(O)N(R′)—. In some embodiments, the alkylene chain is interrupted by, and/or terminates (at either or both termini) with —N(R′)S(O)₂—.

In some embodiments, R₄ is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the polyethylene glycol chain has 1 to 20 —(CH₂CH₂—O—)— units. In some embodiments, the polyethylene glycol chain has 1 to 15 —(CH₂CH₂—O—)— units. In some embodiments, the polyethylene glycol chain has 1 to 10 —(CH₂CH₂—O—)— units. In some embodiments, the polyethylene glycol chain has 1 to 5 —(CH₂CH₂—O—)— units. In some embodiments, the polyethylene glycol chain has 1 to 2 —(CH₂CH₂—O)— units. In some embodiments, the polyethylene glycol is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —C(O)—. In some embodiments, the polyethylene glycol chain is interrupted by, and/or terminates (at either or both termini) with —C(O)O—. In some embodiments, the polyethylene glycol chain is interrupted by, and/or terminates (at either or both termini) with —C(O)N(R′)—. In some embodiments, the polyethylene glycol chain is interrupted by, and/or terminates (at either or both termini) with —N(R′)S(O)₂—.

In some embodiments, n is 2. In some embodiments, n is 2 and each X is CH₂.

In some embodiments, A₂′ is an anti-cancer agent. In some embodiments, A₂′ is an auristatin, a maytansinoid, a tubulysin, an anthracycline, paclitaxel or docetaxel or derivative thereof, calicheamicin or a derivative thereof, pyrrolobenzodiazepine dimer (PBD) or a derivative thereof, duocarmycin or a derivative thereof, eribulin or a derivative thereof, camptothecin or a derivative thereof, or exatecan or a derivative thereof.

In some embodiments, the antibody is a monoclonal antibody, R₁ and R₂, together with the nitrogen atom to which they are attached, form a piperazinyl, and the compound has a structure represented by formula IVa′1:

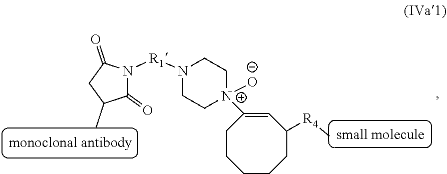

or a pharmaceutically acceptable salt or stereoisomer thereof.

In some embodiments, the antibody is a monoclonal antibody, R₁ is absent and R₂ is methyl, and the compound has a structure represented by formula IVa′2:

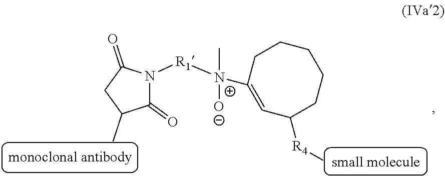

or a pharmaceutically acceptable salt or stereoisomer thereof.

In some embodiments, the compound of formula (V) is represented by a compound of formula (Va):

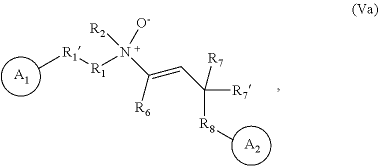

or a pharmaceutically acceptable salt or stereoisomer thereof. In some embodiments, R₁ is absent; R₁′ is an optionally substituted alkylene chain or an optionally substituted polyethylene glycol chain; and/or R₂ is methyl, ethyl, isopropyl, or t-butyl; or R₁′ is CH₂ and R₁ and R₂, together with the nitrogen atom to which they are attached, form a 3- to 16-membered heterocyclyl containing 1-8 heteroatoms selected from N, O, and S; and/or R₆ is hydrogen, chloro, bromo, iodo, OR₁₂, or SR₁₂, wherein each R₁₂ is independently hydrogen, (C₁-C₆) alkyl, (C₃-C₁₀) carbocyclyl, 4- or 7-membered heterocyclyl; and/or R₇ is hydrogen, fluoro, or OR_{5′}, wherein R_{5′} is hydrogen or (C₁-C₆) alkyl; and/or R_{T} is hydrogen, fluoro, or OR_{5′}, wherein R_{5′} is hydrogen or (C₁-C₆) alkyl; and R₈ is CH₂, O, C₆-C₁₂ aryl, or 5- to 10-membered heteroaryl; and/or R₈ is O, S, NR₁₁, OPh, OC(O), OC(O)NR₁₁, wherein R₁₁ is hydrogen or (C₁-C₆) alkyl, an optionally substituted alkylene chain, or an optionally substituted polyethylene glycol chain and/or A₁ is a binding moiety, a therapeutic moiety or a diagnostic moiety; and/or A₂ is a binding moiety, a therapeutic moiety or a diagnostic moiety.

In some embodiments, the compound of formula (V) is

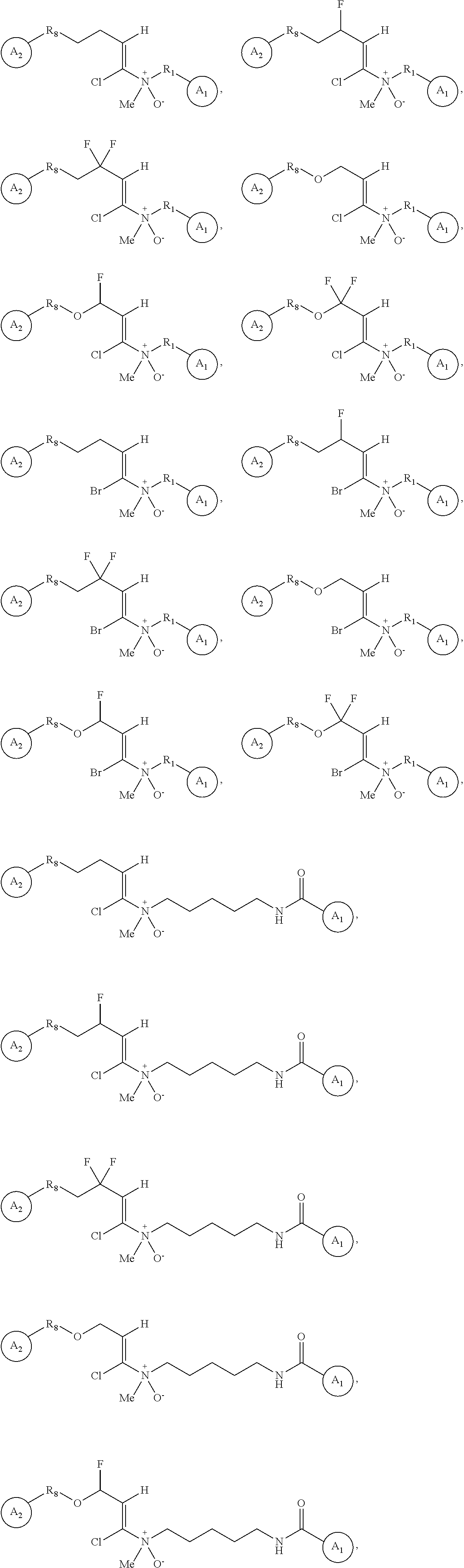

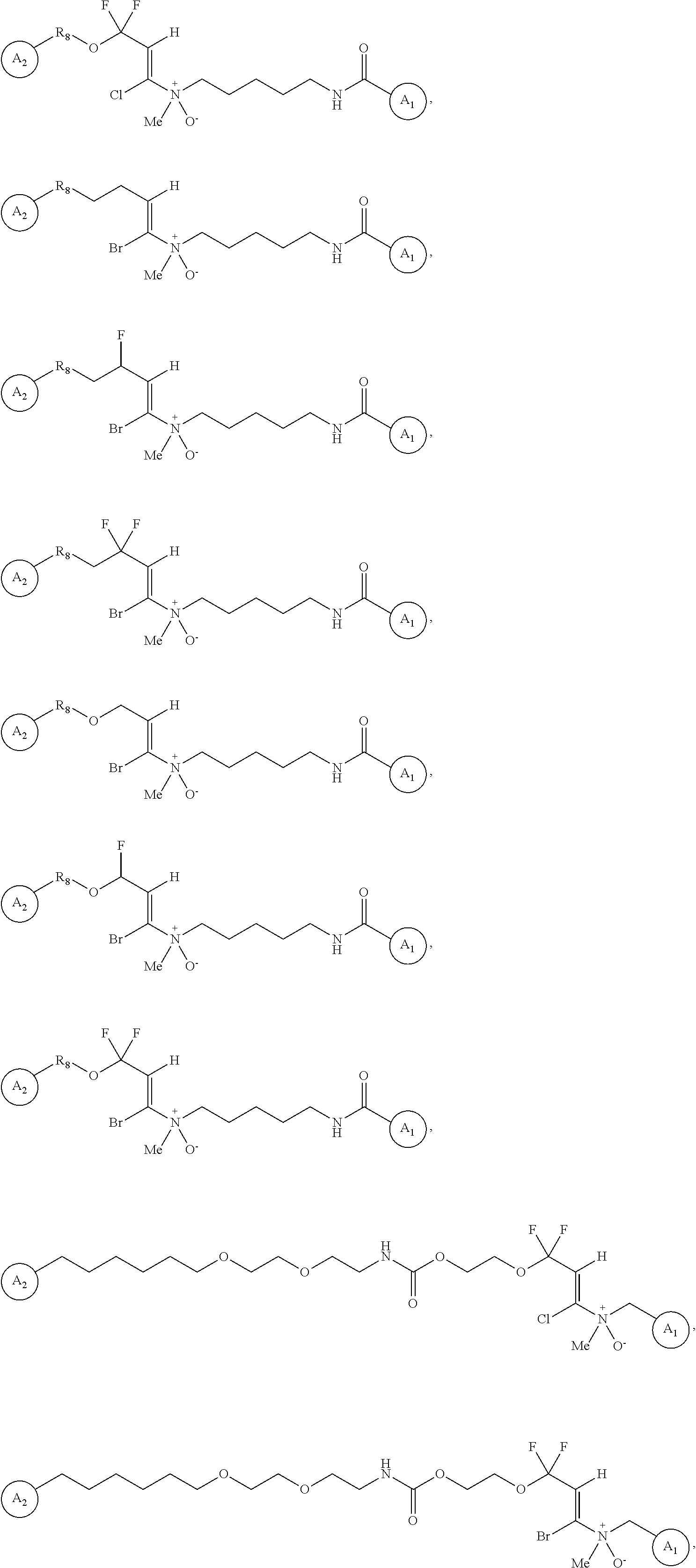

or a pharmaceutically acceptable salt or stereoisomer thereof.

In some embodiments, the compound of formula (V) is

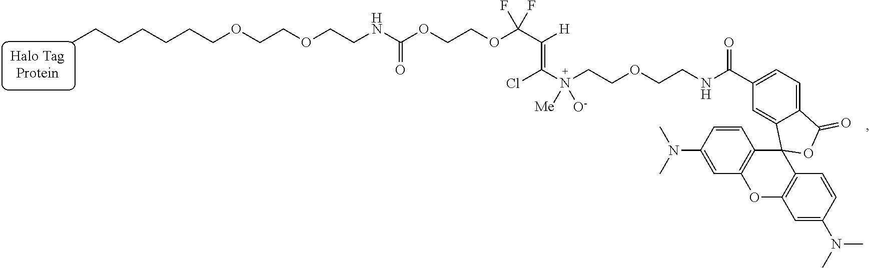

or a pharmaceutically acceptable salt or stereoisomer thereof.

In some embodiments, the optionally substituent for a compound of formula (IV) or (V) is selected from the group comprising of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, and azido.

In embodiments, wherein one of

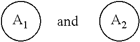

and is a therapeutic moiety and the other is a diagnostic moiety, compounds of formulas (IV) and (V) may be referred to as “theranostic” agents.

In some embodiments, the diagnostic moiety is a fluorophore, and the therapeutic moiety is an anti-cancer agent.

In some embodiments, the diagnostic moiety is a fluorophore, and the therapeutic moiety is a non-targeted anti-cancer agent.

In some embodiments, the diagnostic moiety is a fluorophore, and the therapeutic moiety is a targeted anti-cancer agent.

In some embodiments, the diagnostic moiety is a fluorophore, and the therapeutic moiety is a kinase inhibitor.

In some embodiments, the diagnostic moiety is a fluorophore, and the therapeutic moiety is an anti-bacterial agent.

In some embodiments, the diagnostic moiety is a fluorophore, and the therapeutic moiety is a NSAID.

In some embodiments, the diagnostic moiety is a fluorophore, and the therapeutic moiety is a DMARD.

In some embodiments, the diagnostic moiety is a chromogenic agent, and the therapeutic moiety is an anti-cancer agent.

In some embodiments, the diagnostic moiety is a chromogenic agent, and the therapeutic moiety is a non-targeted anti-cancer agent.

In some embodiments, the diagnostic moiety is a chromogenic agent, and the therapeutic moiety is a targeted anti-cancer agent.

In some embodiments, the diagnostic moiety is a chromogenic agent, and the therapeutic moiety is a kinase inhibitor.

In some embodiments, the diagnostic moiety is a chromogenic agent, and the therapeutic moiety is an anti-bacterial agent.

In some embodiments, the diagnostic moiety is a chromogenic agent, and the therapeutic moiety is a NSAID.

In some embodiments, the diagnostic moiety is a chromogenic agent, and the therapeutic moiety is a DMARD.

In some embodiments, the diagnostic moiety is a PET tracer, and the therapeutic moiety is an anti-cancer agent.

In some embodiments, the diagnostic moiety is a PET tracer, and the therapeutic moiety is a non-targeted anti-cancer agent.

In some embodiments, the diagnostic moiety is a PET tracer, and the therapeutic moiety is a targeted anti-cancer agent.

In some embodiments, the diagnostic moiety is a PET tracer, and the therapeutic moiety is a kinase inhibitor.

In some embodiments, the diagnostic moiety is a PET tracer, and the therapeutic moiety is an anti-bacterial agent.

In some embodiments, the diagnostic moiety is a PET tracer, and the therapeutic moiety is a NSAID.

In some embodiments, the diagnostic moiety is a PET tracer, and the therapeutic moiety is a DMARD.

In some embodiments, the diagnostic moiety is a MRI contrast agent, and the therapeutic moiety is an anti-cancer agent.

In some embodiments, the diagnostic moiety is a MRI contrast agent, and the therapeutic moiety is a non-targeted anti-cancer agent.

In some embodiments, the diagnostic moiety is a MRI contrast agent, and the therapeutic moiety is a targeted anti-cancer agent.

In some embodiments, the diagnostic moiety is a MRI contrast agent, and the therapeutic moiety is a kinase inhibitor.

In some embodiments, the diagnostic moiety is a MRI contrast agent, and the therapeutic moiety is an anti-bacterial agent.

In some embodiments, the diagnostic moiety is a MRI contrast agent, and the therapeutic moiety is a NSAID.

In some embodiments, the diagnostic moiety is a MRI contrast agent, and the therapeutic moiety is a DMARD.

In some embodiments, wherein one of

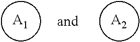

is a binding moiety and the other is a different binding moiety, compounds of formulas (IV) and (V) may be referred to as a proteolysis-targeting chimera (also known as a PROTAC or degrader) that targets a given protein for selective degradation.

In some embodiments, the first binding moiety binds an E3 ubiquitin ligase and the second binding moiety binds ALK. In some embodiments, the E3 ubiquitin ligase is cereblon. In some embodiments, the E3 ubiquitin ligase is VHL. In some embodiments, the E3 ligase is IAP. In some embodiments, the E3 ligase is MDM2.

In some embodiments, the first binding moiety binds an E3 ubiquitin ligase and the second binding moiety binds BTK. In some embodiments, the E3 ubiquitin ligase is cereblon. In some embodiments, the E3 ubiquitin ligase is VHL. In some embodiments, the E3 ligase is IAP. In some embodiments, the E3 ligase is MDM2.

In some embodiments, the first binding moiety binds an E3 ubiquitin ligase and the second binding moiety binds BET. In some embodiments, the E3 ubiquitin ligase is cereblon. In some embodiments, the E3 ubiquitin ligase is VHL. In some embodiments, the E3 ligase is IAP. In some embodiments, the E3 ligase is MDM2.

In some embodiments, the first binding moiety binds an E3 ubiquitin ligase and the second binding moiety binds BRD4. In some embodiments, the E3 ubiquitin ligase is cereblon. In some embodiments, the E3 ubiquitin ligase is VHL. In some embodiments, the E3 ligase is IAP. In some embodiments, the E3 ligase is MDM2.

In some embodiments, the first binding moiety binds an E3 ubiquitin ligase and the second binding moiety binds HDAC. In some embodiments, the E3 ubiquitin ligase is cereblon. In some embodiments, the E3 ubiquitin ligase is VHL. In some embodiments, the E3 ligase is IAP. In some embodiments, the E3 ligase is MDM2.

In some embodiments, the first binding moiety binds an E3 ubiquitin ligase and the second binding moiety binds estrogen receptor. In some embodiments, the E3 ubiquitin ligase is cereblon. In some embodiments, the E3 ubiquitin ligase is VHL. In some embodiments, the E3 ligase is IAP. In some embodiments, the E3 ligase is MDM2.

In some embodiments, the first binding moiety binds an E3 ubiquitin ligase and the second binding moiety binds androgen receptor. In some embodiments, the E3 ubiquitin ligase is cereblon. In some embodiments, the E3 ubiquitin ligase is VHL. In some embodiments, the E3 ligase is IAP. In some embodiments, the E3 ligase is MDM2.

In some embodiments, wherein one of

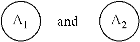

and is a therapeutic moiety and the other is a binding moiety that includes an antibody or a (cellular target) binding fragment thereof, compounds of formulas (IV and IV′) and (V) may be referred to as antibody-drug conjugates. In some embodiments, the therapeutic moiety of the antibody-drug conjugate is an anti-cancer agent.

In some embodiments, the binding moiety of the antibody-drug conjugate is a monoclonal antibody or a fragment thereof, and the therapeutic moiety is a non-targeted anti-cancer agent.

In some embodiments, the binding moiety of the antibody-drug conjugate is a monoclonal antibody or a fragment thereof, and the therapeutic moiety is a targeted anti-cancer agent.

In some embodiments, the binding moiety of the antibody-drug conjugate is a monoclonal antibody or a binding fragment thereof, and the therapeutic moiety is a kinase inhibitor.

In some embodiments the binding moiety of the antibody-drug conjugate is a monoclonal antibody or a binding fragment thereof, and the therapeutic moiety is an anti-bacterial agent.

In some embodiments, the binding moiety of the antibody-drug conjugate is a monoclonal antibody or a binding fragment thereof, and the therapeutic moiety is a NSAID.

In some embodiments, the binding moiety of the antibody-drug conjugate is a monoclonal antibody or a binding fragment thereof, and the therapeutic moiety is a DMARD.

In some embodiments, the binding moiety is biotin or a derivative thereof, and the therapeutic moiety is a targeted anti-cancer agent.

In some embodiments, the binding moiety is biotin or a derivative thereof, and the therapeutic moiety is a kinase inhibitor.

In some embodiments, the binding moiety is biotin or a derivative thereof, and the therapeutic moiety is an anti-bacterial agent.

In some embodiments, the binding moiety is biotin or a derivative thereof, and the therapeutic moiety is a NSAID.

In some embodiments, the binding moiety is biotin or a derivative thereof, and the therapeutic moiety is a DMARD.

Compounds of the present invention may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin. Suitable base salts include aluminum, calcium, lithium, magnesium, potassium, sodium, or zinc salts.

Compounds of the present invention may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

In some embodiments, the compound is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

The compounds of the present invention may be prepared by crystallization under different conditions and may exist as one or a combination of polymorphs of the compound. For example, different polymorphs may be identified and/or prepared using different solvents, or different mixtures of solvents for recrystallization, by performing crystallizations at different temperatures, or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other known techniques.

In some embodiments, the pharmaceutical composition comprises a co-crystal of an inventive compound. The term “co-crystal”, as used herein, refers to a stoichiometric multi-component system comprising a compound of the invention and a co-crystal former wherein the compound of the invention and the co-crystal former are connected by non-covalent interactions. The term “co-crystal former”, as used herein, refers to compounds which can form intermolecular interactions with a compound of the invention and co-crystallize with it. Representative examples of co-crystal formers include benzoic acid, succinic acid, fumaric acid, glutaric acid, trans-cinnamic acid, 2,5-dihydroxybenzoic acid, glycolic acid, trans-2-hexanoic acid, 2-hydroxycaproic acid, lactic acid, sorbic acid, tartaric acid, ferulic acid, suberic acid, picolinic acid, salicyclic acid, maleic acid, saccharin, 4,4′-bipyridine p-aminosalicyclic acid, nicotinamide, urea, isonicotinamide, methyl-4-hydroxybenzoate, adipic acid, terephthalic acid, resorcinol, pyrogallol, phloroglucinol, hydroxyquinol, isoniazid, theophylline, adenine, theobromine, phenacetin, phenazone, etofylline, and phenobarbital.

### Methods of Synthesis

In another aspect, the present invention is directed to a method for making an inventive compound, or a pharmaceutically acceptable salt or stereoisomer thereof. Broadly, the inventive compounds and their pharmaceutically acceptable salts and stereoisomers may be prepared by any process known to be applicable to the preparation of chemically related compounds. The compounds of the present invention will be better understood in connection with the synthetic schemes that are described in various working examples and which illustrate non-limiting methods by which the compounds may be prepared, e.g. compounds of Formulas I-III.

In one of these aspects, the present invention is directed to methods for preparing compounds of formula IV:

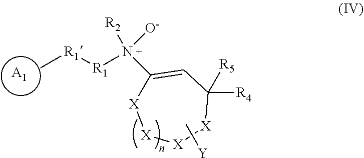

comprising reacting a compound of formula I:

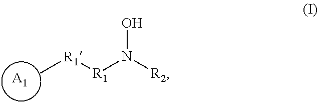

with a compound of formula II:

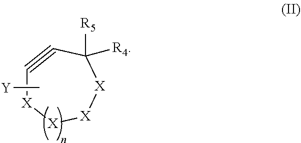

In some embodiments, a compound of formula (I) can be administered together with a compound of formula (II) to form a compound of formula (IV) in vivo.

In one of these aspects, the present invention is directed to methods for preparing compounds of formula V:

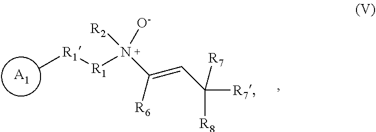

comprising reacting a compound of formula I:

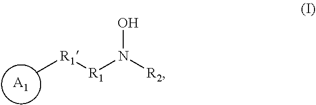

with a compound of formula III:

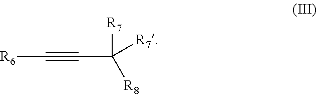

In some embodiments, a compound of formula (I) can be administered together with a compound of formula (III) to form a compound of formula (V) in vivo. Synthetic schemes for attaching active moieties to chemical compounds are known in the art. See, e.g., Agarwal et al., Bioconjugate Chem. 26(2):176-192 (2015).

In one of these aspects, the present invention is directed to methods for preparing compounds of formula IVa′:

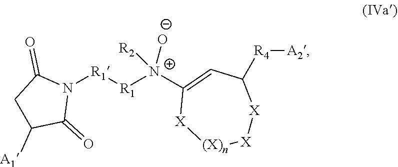

comprising reacting a compound of formula Ia′:

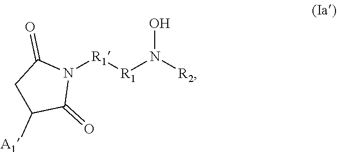

with a compound of formula II′:

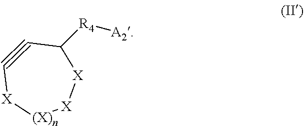

In one of these aspects, the present invention is directed to methods for preparing compounds of formula IVb′:

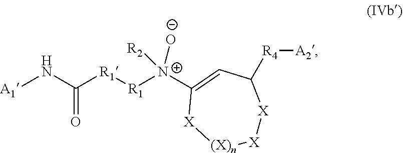

comprising reacting a compound of formula Ib′:

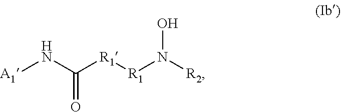

with a compound of formula II′:

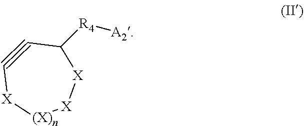

In one of these aspects, the present invention is directed to methods for preparing compounds of formula IVc′:

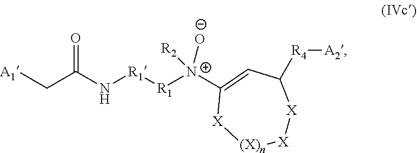

comprising reacting a compound of formula Ic′:

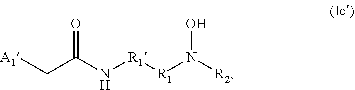

with a compound of formula II′:

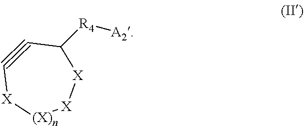

In some embodiments, the reacting is carried out in the presence of a solvent.

In some embodiments, the solvent is an aprotic solvent. In some embodiments, the aprotic solvent is DCM, CHCl₃, CCl₄, DCE, toluene, MeCN, or THF.

In some embodiments, the solvent is a protic solvent. In some embodiments, the protic solvent is MeOH, EtOH, iPrOH, nBuOH, TFE, or HFIP.

In some embodiments, the solvent is a solvent mixture. In some embodiments, the solvent mixture is a mixture of an aprotic solvent and a protic solvent. In some embodiments, the solvent mixture is 0-100% protic to aprotic. In some embodiments, the solvent mixture is 0-1⁰⁰% TFE in CHCl₃. In some embodiments, the solvent mixture is about 20% TFE in CHCl₃.

In some embodiments, the reaction is carried out in the presence of an aqueous buffer.

In some embodiments, the aqueous buffer is an acidic buffer. In some embodiments, the aqueous buffer is an alkaline buffer.

In some embodiments, the reaction is carried out in the presence of a biological fluid.

In some embodiments, the biological fluid is blood, synovial fluid, lymph, or vitrious fluid.

In some embodiments, the reaction is carried out in the presence of an aqueous solution with biological components such as cell lysate, proteins, nucleic acids, or lipids.

In some embodiments, the reaction is carried out with the addition of a buffering reagent. Representative examples of buffered reagents include ascorbic acid, glutathione, citric acid, acetic acid, monopotassium phosphate, N-cyclohexyl-2-aminoethanesulfonic acid (CHES), and borate. In some embodiments, the buffering reagent which is ascorbic acid or glutathione.

In some embodiments, the reaction is carried out at a temperature from about −40° C. to 80° C. In some embodiments, the reacting is carried out at a temperature between 0° C.-60° C. In some embodiments, the reaction is carried out at a temperature of about 60° C. In some embodiments, the reacting is carried out at a temperature is about 20° C.-25° C.

In some embodiments, the compound of formula (I) is in excess of the compound of formula (II) or (III). In some embodiments, the excess is about 10 equivalents. In some embodiments, the excess is about 5 equivalents.

In some embodiments, the reaction is carried out over a week. In some embodiments, the reaction is carried out over five days. In some embodiments, the reaction is carried out over three days. In some embodiments, the reaction is carried out over a period of 24 hours. In some embodiments, the reaction is carried out over a period of 18 hours. In some embodiments, the reaction is carried out over a period of 12 hours. In some embodiments, the reaction is carried out over a period of 6 hours. In some embodiments, the reaction is carried out over a period of 3 hours. In some embodiments, the reaction is carried out over a period of 2 hours. In some embodiments, the reaction is carried out over a period of 1 hour. In some embodiments, the reaction is carried out over a period of 45 minutes. In some embodiments, the reaction is carried out over a period of 30 minutes. In some embodiments, the reaction is carried out over a period of 15 minutes. In some embodiments, the reaction is carried out over a period of 5 minutes. In some embodiments, the reaction is carried out over a period of 1 minute.

### Pharmaceutical Compositions

Another aspect of the present invention is directed to a pharmaceutical composition that includes a therapeutically effective amount of an inventive compound or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compounds of the invention and their pharmaceutically acceptable salts, or stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds are formulated for oral or intravenous administration (e.g., systemic intravenous injection).

Accordingly, compounds of the invention may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of the invention may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of the invention may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The compounds of the invention may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of the invention may be formulated for topical administration which as used herein, refers to administration intradermally by invention of the formulation to the epidermis. These types of compositions are typically in the form of ointments, pastes, creams, lotions, gels, solutions and sprays.

Representative examples of carriers useful in formulating compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of an inventive compound (that contains a therapeutic moiety or which is therapeutic), or a pharmaceutically acceptable salt or stereoisomer thereof that is effective in producing the desired therapeutic response in a patient. The term “therapeutic response” includes amounts of the inventive compound or a pharmaceutically acceptable salt or stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or inhibits the growth of diseased cells.

As used herein, the term, “diagnostically effective amount” refers to an amount of an inventive compound (that contains an amount of the diagnostic moiety), or a pharmaceutically acceptable salt or stereoisomer thereof that is effective in producing the desired detectable response in a patient.

The total daily dosage of the compounds and usage thereof may be decided in accordance with standard medical practice, e.g., by an attending physician using sound medical judgment. The specific therapeutically effective dose for any particular subject will depend upon a variety of factors, including the following: the disease or disorder being treated and the severity thereof (e.g., its present status); the activity of the compound employed; the specific composition employed; the age, body weight, general health, sex and diet of the subject; the time of administration, route of administration, and rate of excretion of the compound employed; the duration of the treatment; drugs used in combination or coincidental with the specific compound employed; and like factors well known in the medical arts (see, for example, Hardman et al., eds., Goodman and Gilman's The Pharmacological Basis of Therapeutics, 10th Edition, McGraw-Hill Press, 155-173, 2001).

Compounds of the invention may be effective over a wide dosage range. In some embodiments, the total daily dosage (e.g., for adult humans) may range from about 0.001 to about 1600 mg, from 0.01 to about 1000 mg, from 0.01 to about 500 mg, from about 0.01 to about 100 mg, from about 0.5 to about 100 mg, from 1 to about 100-400 mg per day, from about 1 to about 50 mg per day, from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, the compound may be administered at a dose in range from about 0.01 mg to about 200 mg/kg of body weight per day. In some embodiments, a dose of from 0.1 to 100, e.g., from 1 to 30 mg/kg per day in one or more dosages per day may be effective. By way of example, a suitable dose for oral administration may be in the range of 1-30 mg/kg of body weight per day, and a suitable dose for intravenous administration may be in the range of 1-10 mg/kg of body weight per day.

### Methods of Use

In some aspects, the present invention is directed to methods of treating a disease or disorder, that entails administration of a therapeutically effective amount of a compound of formula (IV or V) wherein one of

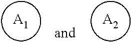

and is a therapeutic agent, or wherein the compound is therapeutic or a pharmaceutically acceptable salt or stereoisomer thereof, to a subject in need thereof. In some embodiments, the disease is cancer.

In some aspects, the present invention is directed to methods of treating cancer, that entail administration of a therapeutically effective amount of a compound of formula IV′ or a pharmaceutically acceptable salt or stereoisomer thereof and a diboron reagent, to a subject in need thereof. In some embodiments, the diboron reagent is a symmetrical diboron reagent. In some embodiments, the diboron reagent is an unsymmetrical diboron reagent. In some embodiments, the diboron reagent is B₂(OH)₄, B₂pin₂,

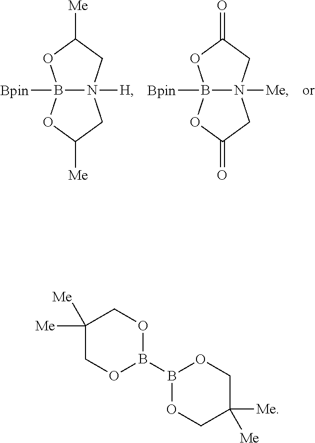

Other representative examples of diboron reagents include bis(catecholato)diboron, bis(hexylene glycolato)diboron, bis[(−)pinanediolato]diboron, bis(diisopropyl-1-tartrate glycolato)diboron, bis(N,N,N′,N′-tetramethyl-d-tartaramide glycolato)diboron, and 2,2′-bi-1,3,2-dioxaborinane. Yet other diboron reagents which may be suitable for use in the present invention are disclosed in Ali et al., Studies in Inorganic Chemistry, “Chapter 1—Chemistry of the diboron compounds” 22:1-57 (2005); Neeve et al., Chem. Rev. 116(16):9091-9161 (2016); Ding et al., Molecules 24(7):1325 (2019). In some embodiments, the diboron reagent is administered at a concentration of about 1 μM to about 1 M. In some embodiments, the diboron reagent is administered at a concentration of about 1 μM to about 100 mM. In some embodiments, the diboron reagent is administered at a concentration of about 1 μM to about 10 mM. In some embodiments, the diboron reagent is administered at a concentration of about 1 μM to about 1 mM. In some embodiments, the diboron reagent is administered at a concentration of about 1 μM to about 100 μM. In some embodiments, the diboron reagent is administered at a concentration of about 1 μM to about 10 μM. In some embodiments, the diboron reagent is administered at a concentration of about 1 μM to about 1 μM. In some embodiments, the diboron reagent is administered at a concentration of about 1 μM to about 100 nM. In some embodiments, the diboron reagent is administered at a concentration of about 1 μM to about 10 nM. In some embodiments, the diboron reagent is administered at a concentration of about 1 μM to about 1 nM. In some embodiments, the diboron reagent is administered at a concentration of about 1 μM to about 100 μM.

In some embodiments, the present methods entail administration of a compound of formula (I) and a compound of formula (II or III), or their pharmaceutically acceptable salts or stereoisomers, wherein one of

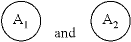

and is a therapeutic agent or wherein the compound formed by reaction between compounds of formulas (I) and (II) and between compounds of formulas (I) and (III) is therapeutic, to a subject in need thereof. The compound of formula (I) and the compounds of formula (II or III) and their pharmaceutically acceptable salts and stereoisomers may be used in combination or concurrently in treating a disease or disorder. The terms “in combination” and “concurrently” in this context mean that the compounds are co-administered, which includes substantially contemporaneous administration, by way of the same or separate dosage forms, and by the same or different modes of administration, or sequentially, e.g., as part of the same regimen. The sequence and time interval may be determined such that they can react together. For example, the compounds may be administered at the same time or sequentially in any order at different points in time; however, if not administered at the same time, they may be administered sufficiently close in time so as to provide the desired therapeutic effect. Therefore, the terms are not limited to the administration of the active agents at exactly the same time. In some embodiments, the methods are directed to treating cancer.

In some aspects, the present invention is directed to methods of both treating and diagnosing a disease or disorder that entail administering a compound of formula (IV or V), or a pharmaceutically acceptable salt or stereoisomer thereof, to a subject in need thereof, wherein the compound is in the form of a theranostic agent. In some embodiments, the disease is cancer.

In some aspects, the present invention is directed to theranostic agents used to treat and diagnose a disease or disorder such as cancer, that entails administration of a compound of formula (I) and a compound of formula (II or III), or their pharmaceutically acceptable salts or stereoisomers, to a subject in need thereof.

In some aspects, the present invention is directed to methods of protein labeling, that entails administration of a compound of formula (IV or V), or a pharmaceutically acceptable salt or stereoisomer thereof, to a subject in need thereof, wherein the compound of formula (IV or V) contains a diagnostic moiety and a binding moiety. In some embodiments, the methods are directed to labeling a cancer associated antigen. Tumor-associated antigens which may be suitable for use in the present invention are disclosed in Ilyas et al., J. Immunol. 195(11):5117-5122 (2015) and Haen et al., Nat. Rev. Clin. Oncol. 17:595-610 (2020).

A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health. In some embodiments, inventive compounds may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

Inventive compounds may be used to treat and/or diagnose a wide variety of diseases and disorders, including cancer and non-cancerous conditions alike. Exemplary types of non-cancerous (e.g., cell proliferative) diseases or disorders that may be amenable to treatment with the compounds of the present invention include inflammatory diseases and conditions, autoimmune diseases, heart diseases, viral diseases, chronic and acute kidney diseases or injuries, metabolic diseases, and allergic and genetic diseases.

Representative examples of specific non-cancerous diseases and disorders include rheumatoid arthritis, alopecia areata, lymphoproliferative conditions, autoimmune hematological disorders (e.g. hemolytic anemia, aplastic anemia, anhidrotic ectodermal dysplasia, pure red cell anemia and idiopathic thrombocytopenia), cholecystitis, acromegaly, rheumatoid spondylitis, osteoarthritis, gout, scleroderma, sepsis, septic shock, dacryoadenitis, cryopyrin associated periodic syndrome (CAPS), endotoxic shock, endometritis, gram-negative sepsis, keratoconjunctivitis sicca, toxic shock syndrome, asthma, adult respiratory distress syndrome, chronic obstructive pulmonary disease, chronic pulmonary inflammation, chronic graft rejection, hidradenitis suppurativa, inflammatory bowel disease, Crohn's disease, Behcet's syndrome, systemic lupus erythematosus, glomerulonephritis, multiple sclerosis, juvenile-onset diabetes, autoimmune uveoretinitis, autoimmune vasculitis, thyroiditis, Addison's disease, lichen planus, appendicitis, bullous pemphigus, pemphigus vulgaris, pemphigus foliaceus, paraneoplastic pemphigus, myasthenia gravis, immunoglobulin A nephropathy, Hashimoto's disease, Sjogren's syndrome, vitiligo, Wegener granulomatosis, granulomatous orchitis, autoimmune oophoritis, sarcoidosis, rheumatic carditis, ankylosing spondylitis, Grave's disease, autoimmune thrombocytopenic purpura, psoriasis, psoriatic arthritis, eczema, dermatitis herpetiformis, ulcerative colitis, pancreatic fibrosis, hepatitis, hepatic fibrosis, CD14 mediated sepsis, non-CD14 mediated sepsis, acute and chronic renal disease, irritable bowel syndrome, pyresis, restenosis, cervicitis, stroke and ischemic injury, neural trauma, acute and chronic pain, allergic rhinitis, allergic conjunctivitis, chronic heart failure, congestive heart failure, acute coronary syndrome, cachexia, malaria, leprosy, leishmaniosis, Lyme disease, Reiter's syndrome, acute synovitis, muscle degeneration, bursitis, tendonitis, tenosynovitis, herniated, ruptured, or prolapsed intervertebral disk syndrome, osteopetrosis, rhinosinusitis, thrombosis, silicosis, pulmonary sarcosis, bone resorption diseases, such as osteoporosis, fibromyalgia, AIDS and other viral diseases such as Herpes Zoster, Herpes Simplex I or II, influenza virus and cytomegalovirus, diabetes Type I and II, obesity, insulin resistance and diabetic retinopathy, 22q11.2 deletion syndrome, Angelman syndrome, Canavan disease, celiac disease, Charcot-Marie-Tooth disease, color blindness, Cri du chat, Down syndrome, cystic fibrosis, Duchenne muscular dystrophy, haemophilia, Klinefleter's syndrome, neurofibromatosis, phenylketonuria, Prader-Willi syndrome, sickle cell disease, Tay-Sachs disease, Turner syndrome, urea cycle disorders, thalassemia, otitis, pancreatitis, parotitis, pericarditis, peritonitis, pharyngitis, pleuritis, phlebitis, pneumonitis, uveitis, polymyositis, proctitis, interstitial lung fibrosis, dermatomyositis, atherosclerosis, arteriosclerosis, amyotrophic lateral sclerosis, asociality, varicosis, vaginitis, depression, and Sudden Infant Death Syndrome.

In some embodiments, the methods are directed to treating subjects having cancer. Generally, the compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), polycythemia vera, lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), germ cell tumor, ovarian germ cell tumor, head and neck cancer, Hodgkin's lymphoma, leukemia, lymphoma, multiple myeloma, hepatocellular carcinoma, hypopharyngeal cancer, intraocular melanoma, ocular cancer, islet cell tumors (endocrine pancreas), renal cancer (e.g., Wilm's Tumor, clear cell renal cell carcinoma), liver cancer, lung cancer (e.g., non-small cell lung cancer and small cell lung cancer), Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, essential thrombocythemia, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, pituitary tumor, plasma cell neoplasm, pleuropulmonary blastoma, prostate cancer, retinoblastoma rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, transitional cell cancer of the renal pelvis and ureter and other urinary organs, urethral cancer, gestational trophoblastic tumor, vaginal cancer and vulvar cancer.

Sarcomas that may be treatable with compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue) and mesenchymous or mixed mesodermal tumor (mixed connective tissue types).

In some embodiments, methods of the present invention entail treatment of subjects having cell proliferative diseases or disorders of the hematological system, liver, brain, lung, colon, pancreas, prostate, ovary, breast, skin, and endometrium.

As used herein, “cell proliferative diseases or disorders of the hematological system” include lymphoma, leukemia, myeloid neoplasms, mast cell neoplasms, myelodysplasia, benign monoclonal gammopathy, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, compounds of the present invention may be used to treat non-metastatic or metastatic lung cancer (e.g., NSCLC, ALK-positive NSCLC, NSCLC harboring ROS1 Rearrangement, Lung Adenocarcinoma, and Squamous Cell Lung Carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell types).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

The compounds of the present invention and their pharmaceutically acceptable salts and stereoisomers may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which have been unsuccessful, or partially successful but who became non-responsive or intolerant to the particular treatment. Therapy may also be given as an adjuvant treatment, i.e., to prevent reoccurrence of cancer in patients with no currently detectable disease or after surgical removal of a tumor. Thus, in some embodiments, the compound may be administered to a patient who has received prior therapy, such as chemotherapy, radioimmunotherapy, surgical therapy, immunotherapy, radiation therapy, targeted therapy or any combination thereof.

The methods of the present invention may entail administration of an inventive compound or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) followed by a 7-day off period. In other embodiments, the compound may be dosed twice a day (BID) over the course of two and a half days (for a total of 5 doses) or once a day (QD) over the course of two days (for a total of 2 doses). In other embodiments, the compound may be dosed once a day (QD) over the course of five days.

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a compound of the present invention or a pharmaceutical composition which contains the compound and a pharmaceutically acceptable carrier wherein the compound and the carrier may be disposed in the same or separate containers. The kits or pharmaceutical systems of the invention may also include printed instructions for using the compounds and compositions.

These and other aspects of the present invention will be further appreciated upon consideration of the following Examples, which are intended to illustrate certain particular embodiments of the invention but are not intended to limit its scope, as defined by the claims.

### Examples

### Example 1: General Information, Materials, and Instrumentations

### General Information

All reactions were conducted in flame-dried round-bottom flasks under a positive pressure of nitrogen unless otherwise stated. Gas-tight syringes with stainless steel needles or cannulae were used to transfer air- and moisture-sensitive liquids. Flash column chromatography was performed using granular silica gel (60-Å pore size, 40-63 μm, Silicycle). Analytical thin layer chromatography (TLC) was performed using glass plates pre-coated with 0.25 mm silica gel impregnated with a fluorescent indicator (254 nm, Silicycle). TLC plates were visualized by exposure to short wave ultraviolet light (254 nm) and/or an aqueous solution of potassium permanganate (KMnO₄). Organic solutions were concentrated at 20° C. on rotary evaporators capable of achieving a minimum pressure of ˜2 torr unless otherwise stated. Room temperature is defined as 22.5±2.5° C. Reaction heating was performed using a UCON™ fluid heating bath.

### General Chemical Materials

All solvents were purchased from Fisher Scientific or Sigma-Aldrich. Unless otherwise stated chemical reagents were purchased from Fisher Scientific, Sigma-Aldrich, Alfa Aesar, Oakwood Chemical, Acros Organics, Combi-Blocks, or TCI America. CMA refers to a solution of 80:18:2 v/v/v chloroform:methanol (MeOH):ammonium hydroxide (28-30% ammonia solution). Chloroform used in CMA solutions and as co-eluents in silica gel column chromatography were stabilized with 0.75% v/v ethanol. Chloroform used in all hydroamination reactions were stabilized with pentene.

### General Chemical Instrumentation

Proton nuclear magnetic resonance (H NMR) spectra, recorded with a 500 MHz Avance III Spectrometer with multi-nuclear Smart probe, are reported in parts per million on the 6 scale, and are referenced from the residual protium in the NMR solvent (CDCl₃: δ 7.24, CD₃OD: δ 3.31 (CHD₂OD), CD₃CN: δ 1.94). Data are reported as follows: chemical shift [multiplicity (s=singlet, d=doublet, t=triplet, dd=doublet of doublets, dt=doublet of triplets, dq=doublet of quartets, ddd=doublet of doublets of doublets, tt=triplet of triplets, td=triplet of doublets, tq=triplet of quatets, m=multiplet), coupling constant(s) in Hertz, integration, assignment]. Carbon-13 nuclear magnetic resonance (¹³C NMR) spectra are referenced from the carbon resonances of the solvent (CDCl₃: δ 77.23, CD₃OD: δ 49.15, CD₃CN: δ 1.37). Fluorine-19 nuclear magnetic resonance (¹⁹F NMR) is calibrated from the fluorine resonances of benzotrifluoride (CDCl₃: δ −62.76, CD₃OD: δ −64.24, CD₃CN: 6-63.22). Data are reported as follows: chemical shift (assignment). Infrared data (IR) were obtained with a Cary 630 Fourier transform infrared spectrometer equipped with a diamond ATR objective and are reported as follows: frequency of absorption (cm⁻¹), intensity of absorption (s=strong, m=medium, w=weak, br=broad). High resolution mass spectra (HIRMS) were recorded on a Q Exactive™ Plus Hybrid Quadrupole-Orbitrap™ Mass Spectrometer using an electrospray ionization (ESI), atmospheric pressure ionization (API), or electron ionization (EI) source. Automated C₁₈ reverse phase chromatography was performed using an Isolera™ One (Biotage®) purification system. High performance liquid chromatography (HPLC) purification was performed using an Agilent 1260 Infinity system. In-gel fluorescence imaging was performed on a GE Healthcare Life Sciences Typhoon™ FLA 9500. Images were processed with Fiji ImageJ software.

### General Biological Materials and Methods

All solvents and reagents were purchased from commercial suppliers and used as received. Deionized water (>18.2 μΩ) was used to prepare all aqueous buffers and solutions. Short oligonucleotide primers (<80 bp) were synthesized by MilliporeSigma (St. Louis, MO) while gene blocks (>80 bp) were synthesized by Twist Bioscience (South San Francisco, CA). Oligonucleotides were used as received without further desalting. Chemically competent E. coli DH5α and BL21(DE3) cells were purchased from New England Biolabs®. All plasmid isolations were performed with a miniprep or midiprep kit from Zymo Research. DNA clean and concentrator and DNA gel purification kits were purchased from Zymo Research. All enzymes used for standard restriction enzyme cloning (Q5@ Hot Start DNA polymerase, restriction enzymes, T4 DNA ligase, and Antarctic phosphatase), the NEBuilder® HiFi DNA Assembly master mix for Gibson Assembly® cloning, and the Q5@ mutagenesis kit used to perform all site-directed mutagenesis reactions were purchased from New England Biolabs®. DNA sequencing service was performed by Quintarabio (Cambridge, MA). TransIT®-293 transfection reagent was purchased from Mirus Bio™.

### General Biological Instrumentation

All polymerase chain reactions (PCR) were performed on a Bio-Rad Laboratories C1000 thermal cycler. Cells were lysed using a Fisherbrand™ Sonicator Model 505. Proteins were purified by a Bio-Rad NGC Chromatography System. UV/vis absorbance measurements for protein A280 determination were acquired on an Agilent Technologies Cary 60 UV-Vis. In-gel fluorescence imaging was performed on a GE Healthcare Life Sciences Typhoon™ FLA 9500. Coomassie stained gels were analyzed on a Bio-Rad Molecular Imager Gel Doc XR+ Imaging System.

### Example 2: Bioorthogonal Reactions of Cycloalkynes

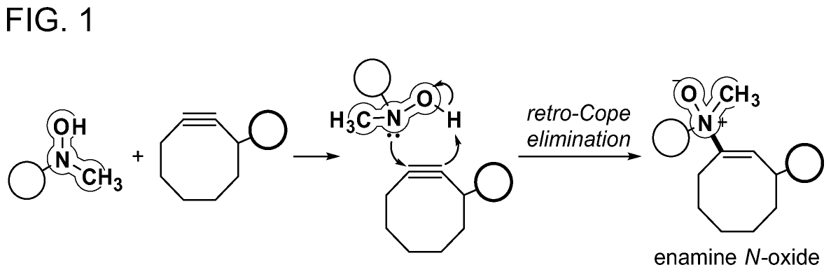

The retro-Cope elimination reaction proved to be useful in biorthogonal reactions (FIG. 1) (Bourgeois et al., J. Am. Chem. Soc. 131(3):874-875 (2009); Beauchemin, A. M., Org. Biomol. Chem. 11:7039-7050 (2013); O'Neil et al., Chem. Commun. 50:7336-7339 (2014)). The bioorthogonal reaction of N,N-dialkyhydroxylamines and cyclooctynes to form stable enamine N-oxide ligation products in a rapid and regioselective manner with reaction components comprising as few as three non-hydrogen atoms is described below.

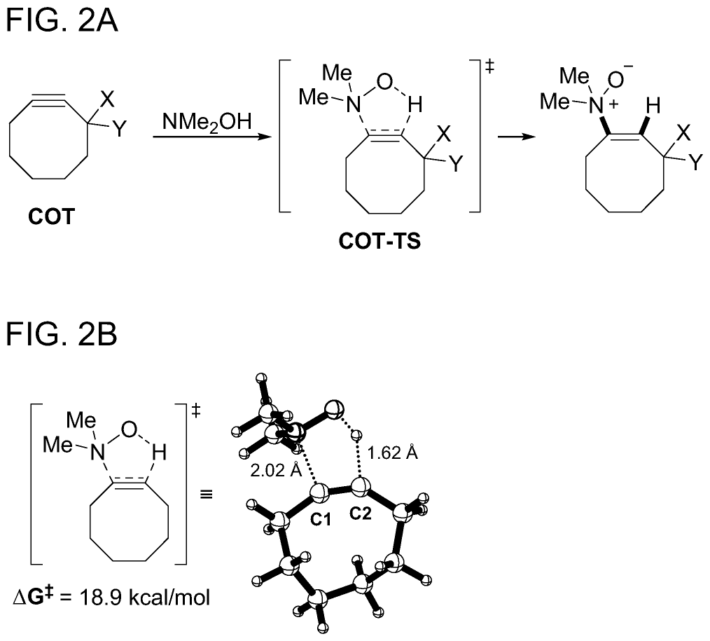

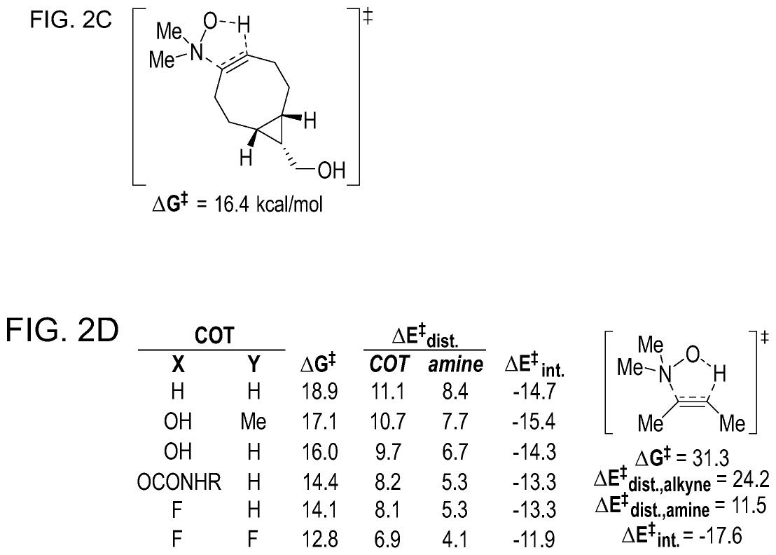

The retro-Cope elimination reaction was evaluated through density functional theory calculations (Zhao et al., Theor. Chem. Acc. 120:215-241 (2008)) and ascertained the activation barriers for the reaction of N,N-dimethylhydroxylamine with a variety of cyclooctynes (FIG. 2A). The calculated activation energy of unmodified cyclooctyne was 18.9 kcal/mol—which was sufficiently low enough for the reaction to proceed at room temperature. The absence of steric factors impinging on the incipient O·H·C2 bond in the transition state structure was equally noteworthy as it portended the importance that steric ambivalence toward propargylic substituents would have on the adaptability, mutual orthogonality, and reactivity of the cyclooctynes.

Calculations of bicyclo[6.1.0]nonyne showed that additional strain could be harnessed to the same effect as for cycloaddition reactions (FIG. 2C) (Dommerholt et al., Angew. Chem. Int. Ed. 49(49):9422-9425 (2010)). Instead, the electronic modulation (Baskin et al., Proc. Natl. Acad. Sci. U.S.A. 104(43):16793-16797 (2007); Agard et al., ACS Chem. Biol. 1(10):644-648 (2006)) of cyclooctyne proved to be more profound (FIG. 2D). Further distortion/interaction energy analysis indicated a sizable reduction in distortion energy (Ess et al., Org. Lett. 10(8):1633-1636 (2008); Liu et al., Acc. Chem. Res. 50(9):2297-2308 (2017)) for the cyclooctynes versus their linear counterpart, but unlike for cycloadditions, this reaction does not benefit from enhancement of interaction energy upon addition of electronegative substituents; the rate acceleration was driven by a decrease in the distortion energy of both components. The counterintuitive increase in the interaction energy was likely due to the reactant-ward shift of the transition state in accordance with Hammond's postulate. Commensurate increases in the lengths of the Cl·N and C2·H bonds further supported this interpretation (Example 30).

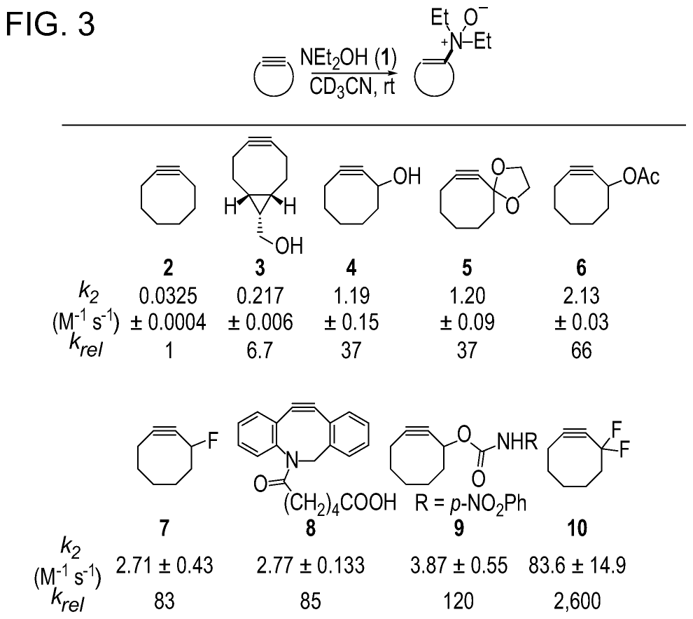

Kinetics experiments corroborated the reactivity trends predicted by computation. Using NMR spectroscopy to monitor reaction progress, the second order rate constants for the reaction of N,N-diethylhydroxyl amine (1) with a panel of cyclooctynes 2-10 in d₃-acetonitrile was determined at room temperature (FIG. 3). Cyclooctyne (2) proved remarkably reactive, displaying a second order rate constant of 3.25×10⁻² M⁻¹s⁻¹ —an order of magnitude faster than its reaction with benzyl azide (Agard et al., J. Am. Chem. Soc. 126(46):15046-15047 (2004)). Further strain enhancements provided a 6.7-fold rate acceleration as predicted for bicyclo[6.1.0]nonyne 3, but far from the 100-fold increase observed for analogous azide-alkyne cycloadditions (Dommerholt et al., Angew. Chem. Int. Ed. 49(49):9422-9425 (2010)). Still, the second order rate constant of 2.17×10⁻¹ M⁻¹s⁻¹ compared favorably with the fastest azide-based reactions involving BARAC (Jewett et al., J. Am. Chem. Soc. 132(11):3688-3690 (2010)).

The hydroamination reaction of cyclooctynes was particularly sensitive to the inductive effects of propargylic substituents, and progressive rate enhancements were observed with increasing electronegativity (FIG. 3). The most reactive of the cyclooctynol-derived substrates was carbamate 9 featuring a rate constant of 3.87 M⁻¹s⁻¹, a 120-fold improvement over that of cyclooctyne (2). Importantly, the minimalistic cyclooctyn-1-ol substructure proved versatile, being both easy to synthesize and derivatize; it was amenable to conjugation via an ester, carbamate, or a ketal linkage without incurring significant costs in size or reactivity. Indeed, elaborate functionalization of the core cyclooctyne was not only unnecessary, it at times proved deleterious. Reaction of N,N-diethylhydroxylamine with dibenzoazacyclooctyne 8 (DIBAC) (Debets et al., Chem. Commun. 46:97-99 (2010)) was rapid, yet still inferior in reaction rate to carbamate 9 and not appreciably superior to its more austere counterparts. Notably, the ligation product of the dibenzoazacyclooctyne 8 plus N,N-diethylhydroxylamine was prone to degradation, being uniquely unstable to purification by both standard and reverse phase flash chromatography.

Prior reports that difluorocyclooctyne 10 operates at the limits of bioorthogonality (Baskin et al., Proc. Natl. Acad. Sci. U.S.A. 104(43):16793-16797 (2007); Kim et al., Carbohydr. Res. 377:18-27 (2013)) and this provided a reasonable upper bound on hydroamination kinetics that could be achieved using electronically-tuned cyclooctynes in biological settings.

Astoundingly, a competition experiment with cyclooctyne 9 revealed its rate constant to be 83.6 M⁻¹s⁻¹.

The retro-Cope elimination reaction is highly directed by substrate electronics and produced only a single observable regioisomer for cyclooctynes 2-7, 9, and 10. Accordingly, when a symmetrical N,N-dialkylhydroxylamine was employed, a single product formed selectively.

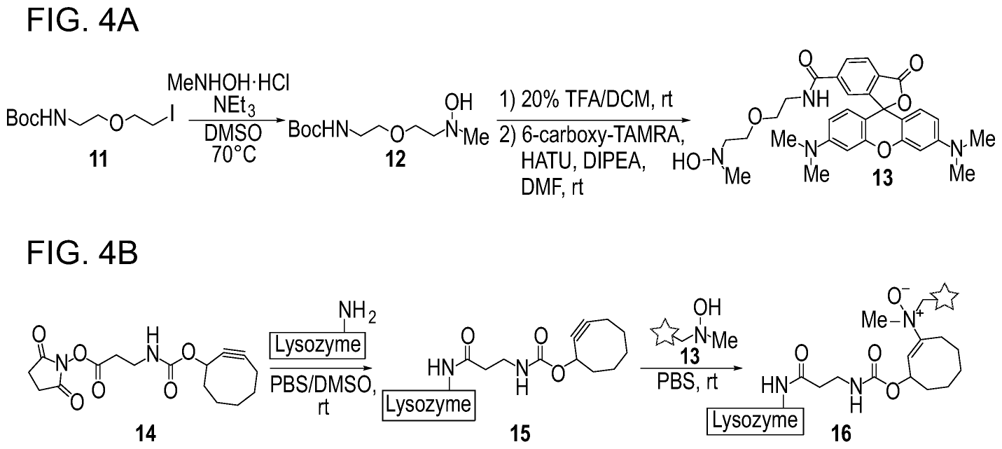

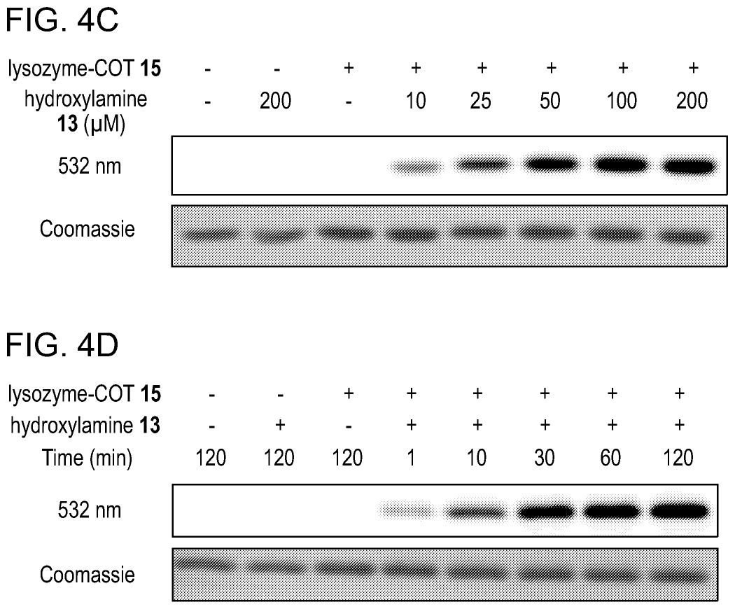

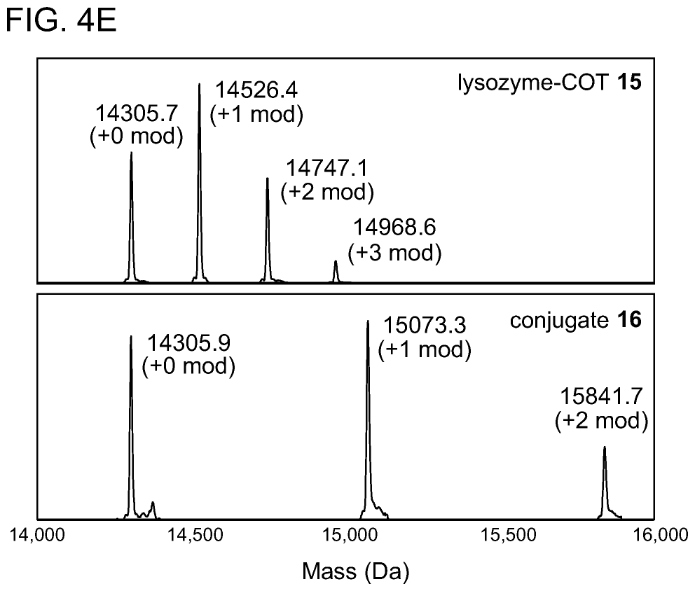

To test the bioorthogonality of the hydroamination reaction, in vitro protein labeling experiments were performed. Fluorophore-conjugated hydroxylamine 13 was first assembled from 6-carboxytetramethylrhodamine and hydroxylamine 12, which was in turn synthesized by nucleophilic displacement of iodide 11 with N-methylhydroxylamine hydrochloride (FIG. 4A). Separately, lysozyme was functionalized with cyclooctyne via N-hydroxysuccinimide ester 14 (FIG. 4B). With both reaction components in hand, cyclooctyne-functionalized lysozyme 15 was treated with hydroxylamine 13 (0-200 μM) in PBS for 2 hours and analyzed by in-gel fluorescence (FIG. 4C). Labeling occurred in a concentration-dependent manner, and labeling was saturated at 100 μM hydroxylamine. The reaction occurred in a time-dependent manner (FIG. 4D). Modified lysozyme 15 was treated with hydroxylamine 13 (200 μM) and quenched with N,N-diethylhydroxylamine (20 mM) at various time points. In-gel fluorescence analysis revealed signal saturation by 1 hour. The desired adducts that formed on the protein were verified by mass spectrometry. Lysozyme 15 was incubated with hydroxylamine 13 (100 μM) in PBS, and the complete conversion of mono- and dicyclooctyne functionalized lysozymes 15 to mono- and dienamine N-oxides 16 was verified by ESI-MS (FIG. 4E).

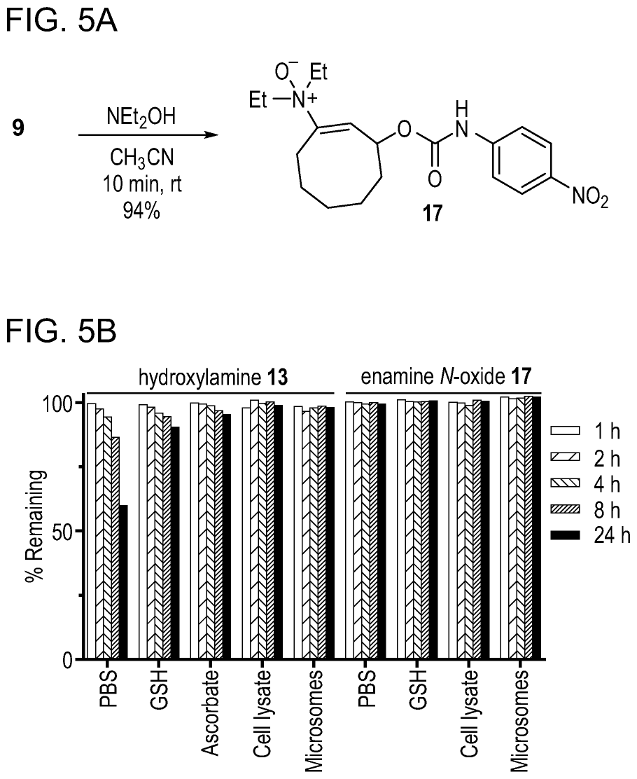

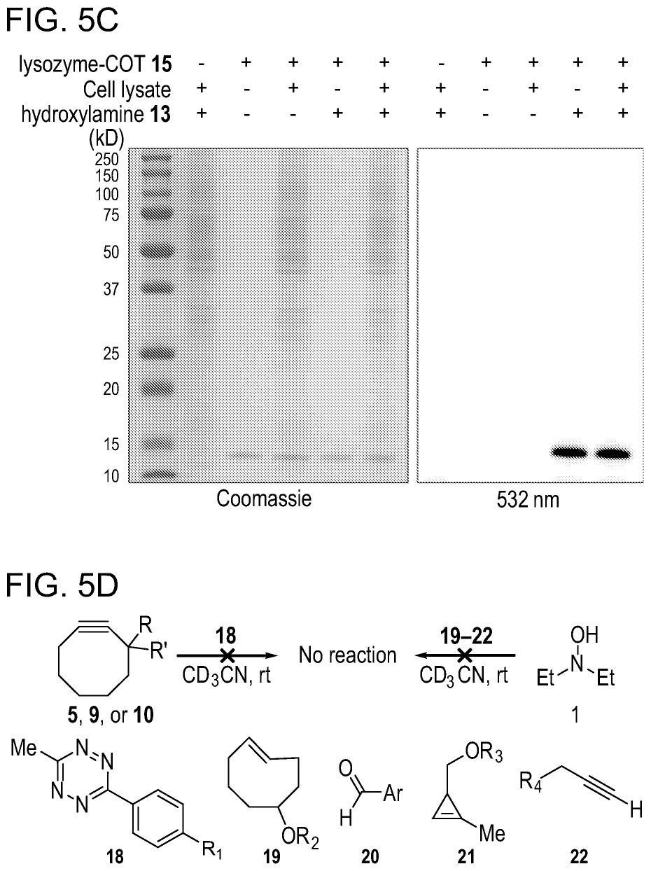

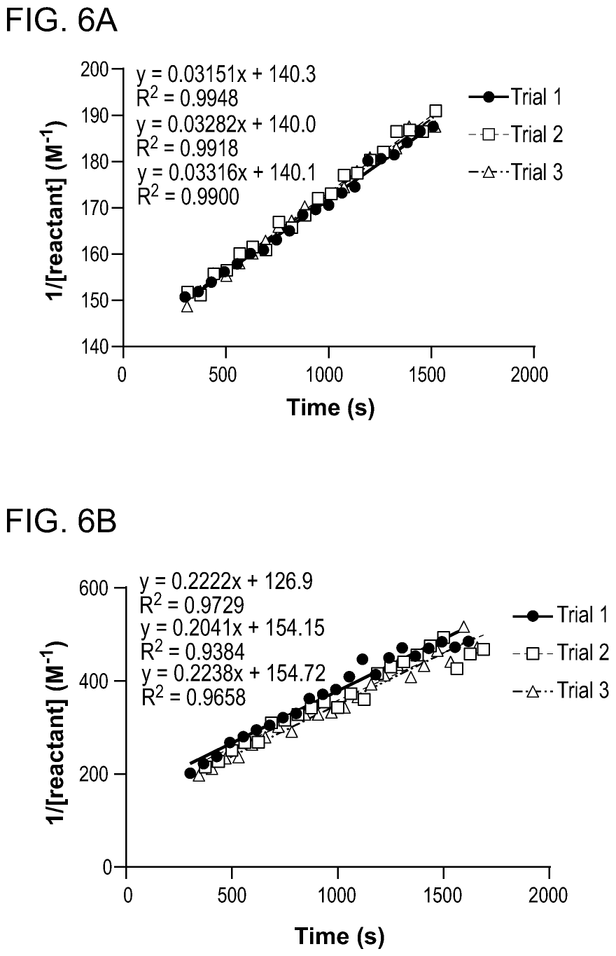

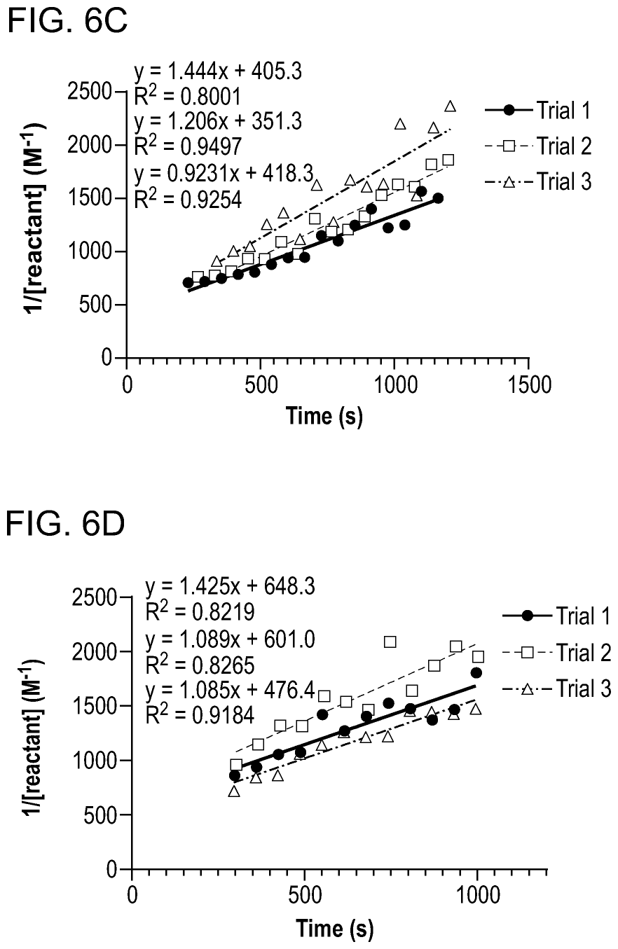

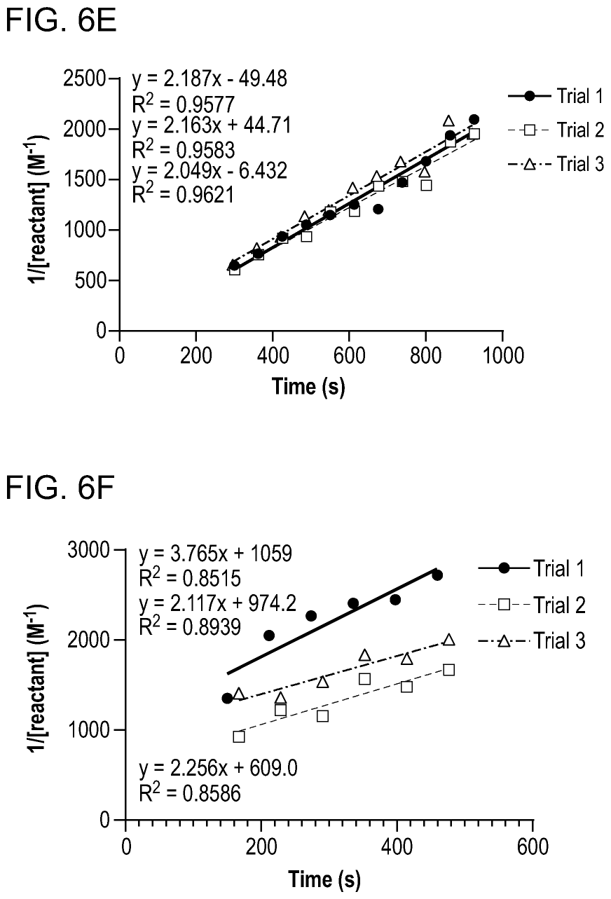

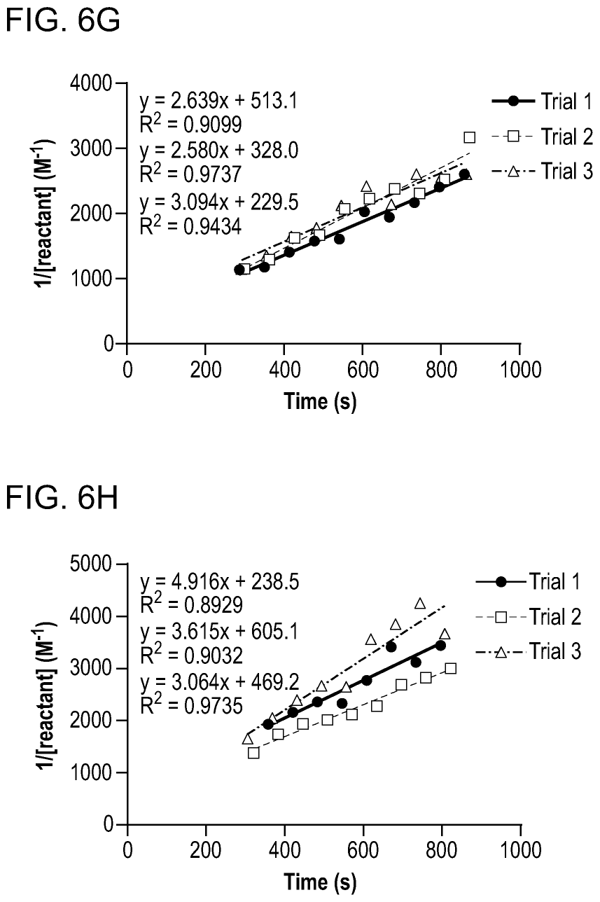

The stability of both the enamine N-oxide and hydroxylamine species under a variety of biologically relevant conditions were verified at various time points (FIG. 5A-FIG. 5B). Hydroxylamine 13 was first incubated in PBS at room temperature, and HPLC analysis of the solution indicated that the compound was >86% intact for up to 8 hours. However, approximately 40% of the hydroxylamine had decomposed by the 24 hour time point. The primary degradation products were consistent with hydrolysis of the regioisomeric nitrones that were likely generated by autoxidation. Consequently, this degradation pathway could be abrogated by the addition of cellular reductants such as ascorbic acid (5 mM) or glutathione (5 mM) (Bobko et al., Free Radical Biol. Med. 42(3):404-412 (2007)). Negligible degradation was observed over 24 hours. Hydroxylamine 13 was stable in HEK293T cell lysate (1 mg/mL), which experienced no degradation above background over 24 hours even when unbuffered with exogenous reductants.

As with the hydroxylamine, the stability of enamine N-oxide 17 was evaluated by HPLC under biologically relevant conditions. It showed no evidence of degradation alone or in the presence of 5 mM glutathione in PBS at room temperature over the course of 24 hours. Furthermore, while N-oxides do undergo reduction in a hemeprotein-dependent manner under hypoxic conditions, this process is sufficiently inhibited by aerobic conditions (Raleigh et al., Int. J. Radiat. Oncol. Biol. Phys. 42(4):763-767 (1998)). Incubation of enamine N-oxide 17 with human liver microsomes (0.2 mg/mL) under ambient air resulted in negligible degradation after 24 hours.

Further demonstrating the bioorthogonality of the reaction, TAMRA-hydroxylamine 13 and lysozyme-COT 15 was combined in the presence and absence of HEK293T cell lysate in PBS for 2 hours (FIG. 5C). In-gel fluorescence showed that the lysozyme was labeled exclusively and that the degree of labeling was unperturbed by the presence of lysate. There appeared to be no cross-reactivity between dialkylhydroxylamine and other proteins under these conditions.

Finally, the cross-compatibility of this reaction with other bioorthogonal systems was explored to identify mutually orthogonal substrate combinations that could be used in tandem (FIG. 5D) (Patterson et al., Curr. Opin. Chem. Biol. 28:141-149 (2015)). Tetrazines were first evaluate to see if they would be compatible with sterically congested cyclooctynes featuring tetrasubstitution at the propargylic position. Indeed, no product could be detected when cyclooctyne ketal 5 and tetrazine 18 were combined at 5 mM concentrations for 1 hour in d₃-acetonitrile. To determine whether steric constraints imposed by the fully substituted carbon (Liu et al., J. Am. Chem. Soc. 136(32):11483-11493 (2014)) or the electronics of the ketal were primarily responsible for inhibiting the inverse-electron demand cycloaddition, electron-deficient cyclooctynes 9 and 10 were evaluated under the same conditions to similar effect. Electronics, alone or in combination with sterics, render the two reactions orthogonal. The hydroxylamine reagents were evaluated to see if they would be compatible with strained alkenes. 5 mM N,N-diethylhydroxylamine (1) combined with 5 mM cyclopropene 21 (Patterson et al., J. Am. Chem. Soc. 134(45):18638-18643 (2012)) or trans-cyclooctene 19 (Blackman et al., J. Am. Chem. Soc. 130(41):13518-13519 (2008)) proved unreactive in d₃-acetonitrile. N,N-dialkylhydroxylamines do not react with aldehydes or engage in the copper-catalyzed azide-alkyne cycloaddition (Example 29) (Hein et al., Chem. Soc. Rev. 39:1302-1315 (2010)).

A new bioorthogonal ligation reaction between N,N-dialkylhydroxylamines and cyclooctynes was identified. The reaction featured rapid kinetics with second order rate constants as high as 84 M⁻¹s⁻¹, exquisite regioselectivity, and small reaction components. The N,N-dialkylhydroxylamine reagent can be pared down to as few as three non-hydrogen atoms, and the cyclooctyne was supremely effective even when unfunctionalized. Cyclooctynes can be attached conveniently at their propargylic positions without incurring costs to reactivity. The hydroxylamine reagent and enamine N-oxide product were sufficiently stable under aqueous conditions in the presence of thiols or components of the cellular milieu found in the cell lysate, particularly on timescales that are germane to the ligation of small molecules to biomolecules. Both components, however, have their sensitivities: hydroxylamines to air and enamine N-oxides to microsomes absent oxygen. Factors that mitigate against these processes were identified and ensured the bioorthogonality of the reaction.

### Example 3: Synthesis of (E)-(cyclooct-1-en-1-yloxy)trimethylsilane

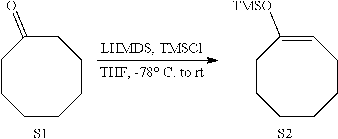

A round-bottom flask was charged sequentially with tetrahydrofuran (THF, 200 mL) and a solution of lithium bis(trimethylsilyl)amide (1 M in THF, 52.3 mL, 52.3 mmol) then cooled to −78° C. A solution of cyclooctanone S1 (6.00 g, 47.5 mmol) in THF (200 mL) was added to the solution at −78° C. via cannula over 20 minutes. After 1.5 hours, chlorotrimethylsilane (TMSCl, 5.94 g, 54.7 mmol) was added and the dry ice bath was removed. The solution was allowed to warm to room temperature. After 1 hour, the reaction was quenched with saturated aqueous ammonium chloride (500 mL) and diluted with hexanes (500 mL). The organic layer was washed with brine (200 mL), dried over anhydrous magnesium sulfate, filtered, and concentrated under reduced pressure. Crude S2 was used in the next step without further purification.

### Example 4: Synthesis of 2-((trimethylsilyl)oxy)cyclooctan-1-one (S3)

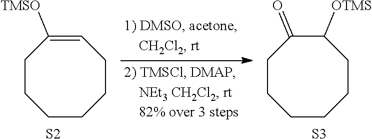

A round-bottom flask was charged with crude S2 from the previous step (47.5 mmol) and dissolved in dichloromethane (DCM, 200 mL). A solution of dimethyldioxirane (DMDO, 0.11 M in acetone, 595 mL, 60.0 mmol) was added to the solution at room temperature. After 15 minutes, the reaction mixture was concentrated and azeotroped with MeOH (2×200 mL). The resulting oil was dissolved in DCM (500 mL). 4-Dimethylaminopyridine (DMAP, 581 mg, 4.75 mmol), triethylamine (9.94 mL, 71.3 mmol), and chlorotrimethylsilane (7.24 mL, 57.1 mmol) were sequentially added to the solution at room temperature. After 2.5 hours, the reaction mixture was washed with aqueous HCl (1 N, 500 mL), the organic layer was separated, and the aqueous layer was extracted with DCM (75 mL). The combined organic layers were dried over anhydrous sodium sulfate, filtered, and concentrated under reduced pressure. The crude mixture was purified by flash column chromatography on silica gel (eluent: 5% ethyl acetate in hexanes) to provide ketone S3 (8.35 g, 82% over 3 steps). ¹H NMR (500 MHz, CDCl₃, 25° C.): δ 4.15 (dd, J=6.9, 3.4 Hz, 1H), 2.65-2.54 (m, 1H), 2.32-2.20 (m, 1H), 2.13-2.02 (m, 1H), 2.03-1.93 (m, 1H), 1.86-1.79 (m, 1H), 1.78-1.63 (m, 2H), 1.57-1.37 (m, 4H), 1.30-1.16 (m, 1H), 0.08 (s, 9H). ¹³C NMR (126 MHz, CDCl₃, 25° C.): δ 217.6, 77.7, 39.1, 34.7, 27.2, 26.1, 25.3, 21.4, 0.2. FTIR (thin film) cm⁻¹: 2930 (b), 1707 (w), 1252 (m), 1111 (m), 1051 (m), 835 (s). HRMS (ESI) (m/z): calc'd for C₁₁H₂₃O₂Si [M+H]⁺: 215.1467, found: 215.1463. TLC (5% ethyl acetate in hexanes), Rf: 0.70 (I₂).

### Example 5: Synthesis of (E)-8-((trimethylsilyl)oxy)cyclooct-1-en-1-yl trifluoromethanesulfonate (S4)

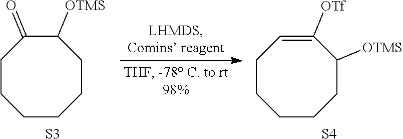

A round-bottom flask was charged sequentially with cyclooctanone S3 (2.06 g, 9.61 mmol) and THF (100 mL) then cooled to −78° C. A solution of lithium bis(trimethylsilyl)amide (1 M in THF, 11.5 mL, 11.5 mmol) at −78° C. was added to the mixture via cannula. After 1 hour, N-(5-chloro-2-pyridyl)bis(trifluoromethanesulfonimide) (4.15 g, 10.6 mmol) was added, and the dry ice bath was removed. After 2 hours, the reaction mixture was diluted with hexanes (200 mL) and washed sequentially with aqueous sodium hydroxide (1 M, 2×150 mL) and brine (100 mL). The resulting organic layer was dried over anhydrous sodium sulfate, filtered, and concentrated under reduced pressure. The crude mixture was purified by flash column chromatography on silica gel (eluent: 7.5% DCM in hexanes) to provide vinyl triflate S4 (3.1 g, 95%) as a colorless oil. ¹H NMR (500 MHz, CDCl₃, 25° C.): δ 5.68 (t, J=9.0 Hz, 1H), 4.66 (dd, J=10.3, 5.4 Hz, 1H), 2.37-2.21 (m, 1H), 2.11-1.97 (m, 1H), 1.83-1.67 (m, 4H), 1.65-1.53 (m, 1H), 1.52-1.29 (m, 3H), 0.14 (s, 9H). ¹³C NMR (126 MHz, CDCl₃, 25° C.): δ 150.6, 118.8 (q, J=319.6 Hz), 120.0, 67.3, 37.0, 29.9, 26.2, 24.8, 23.6, −0.1. ¹⁹F NMR (471 MHz, CDCl₃, 25° C.): 6-75.2. FTIR (thin film) cm⁻¹: 2933 (w), 1416 (m), 1200 (s), 1144 (m), 932 (m), 839 (s). HRMS (ESI) (m/z): calc'd for C₁₂H₂₁F₃NaO₄SSi [M+Na]⁺: 369.0774, found: 369.0776. TLC (100% hexanes), Rf: 0.42 (I₂).

### Example 6: Synthesis of cyclooct-2-yn-1-ol (4)

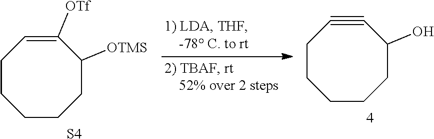

A round-bottom flask was charged sequentially with vinyl triflate S4 (3.03 g, 8.75 mmol) and THF (88 mL) then cooled to −78° C. A solution of lithium diisopropylamide (2.0 M in THF/heptane/ethylbenzene, 8.75 mL, 17.5 mmol) was then added to the solution via syringe. The dry ice bath was removed, and the solution was allowed to warm to room temperature. After 2.5 hours, tetrabutylammonium fluoride (1 M in THF, 17.5 mL, 17.5 mmol) was added to the reaction mixture via syringe. After 1 hour, the reaction mixture was diluted with hexanes (100 mL) and washed with saturated aqueous ammonium chloride (100 mL) and brine (100 mL). The organic layer was dried over anhydrous sodium sulfate, filtered, and concentrated under reduced pressure. The crude mixture was purified by flash column chromatography on silica gel (eluent: 100% DCM) to provide cyclooctynol 4 (566 mg, 52%) as a clear, colorless oil. The physical properties and spectral data were identical to those reported in the literature (Hagendorn, T., Eur. J. Org. Chem. 2014(6):1280-1286 (2014)). TLC (100% DCM), Rf: 0.31 (KMnO₄).

### Example 7: Synthesis of (E)-8-oxocyclooct-1-en-1-yl trifluoromethanesulfonate (S5)

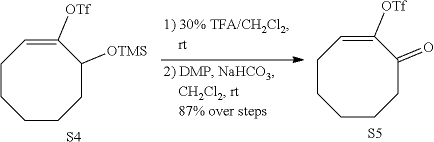

A round-bottom flask was charged sequentially with vinyl triflate S4 (94.3 mg, 272 μmol) and DCM (1.4 mL). Trifluoroacetic acid (600 μL) was added to the solution at room temperature. After 30 minutes, the reaction mixture was concentrated under reduced pressure. The crude mixture was dissolved in DCM (2.7 mL). Sodium bicarbonate (68.6 mg, 817 μmol) and Dess-Martin periodinane (DMP, 231 mg, 544 μmol) were sequentially added at room temperature. After 30 minutes, the reaction mixture was diluted with hexanes (2 mL) and purified by flash column chromatography on silica gel (eluent: 15% ethyl acetate in hexanes) to provide cyclooctenone S5 (64.1 mg, 87%) as a clear thin film. ¹H NMR (500 MHz, CDCl₃, 25° C.): δ 6.58 (t, J=9.2 Hz, 1H), 2.85 (t, J=7.3 Hz, 2H), 2.70 (dt, J=9.3, 7.0 Hz, 2H), 1.84-1.75 (m, 2H), 1.74-1.68 (m, 2H), 1.61-1.54 (m, 2H). ¹³C NMR (126 MHz, CDCl₃, 25° C.): δ 192.8, 149.7, 133.6, 118.8 (q, J=320.1 Hz), 40.7, 25.3, 23.5, 23.1, 22.0. ¹⁹F NMR (471 MHz, CDCl₃, 25° C.): 6-74.2. FTIR (thin film) cm⁻¹: 2937 (w), 1685 (m), 1416 (s), 1200 (s), 1141 (s), 1062 (s), 969 (s). HRMS (ESI) (m/z): calc'd for C₉H₁₂F₃O₄S [M+H]⁺: 273.0403, found: 273.0402. TLC (15% ethyl acetate in hexanes), Rf: 0.30 (KMnO₄).

### Example 8: Synthesis of (E)-1,4-dioxaspiro[4.7]dodec-6-en-6-yl trifluoromethanesulfonate (S6)

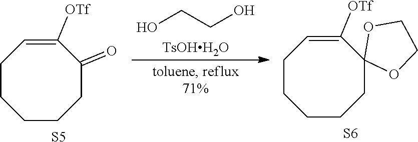

A round-bottom flask was sequentially charged with cyclooctenone S5 (150 mg, 551 μmol), ethylene glycol (302 μL, 5.51 mmol), and benzene (10 mL) at room temperature. p-Toluenesulfonic acid monohydrate (10.5 mg, 55.1 μmol) was then added to the solution. The flask was fitted with a Dean-Stark trap and reflux condenser, and the reaction mixture was heated to reflux. After 23 hours, the reaction mixture was cooled to room temperature and diluted with hexanes. The crude mixture was purified by flash column chromatography on silica gel (eluent: 5% ethyl acetate in hexanes) to provide ketal S6 (125 mg, 71%) as a clear, colorless oil. ¹H NMR (500 MHz, CDCl₃, 25° C.): δ 5.75 (t, J=9.5 Hz, 1H), 4.14-3.91 (m, 4H), 2.48-2.36 (m, 2H), 2.04-1.98 (m, 2H), 1.65-1.52 (m, 6H). ¹³C NMR (126 MHz, CDCl₃, 25° C.): δ 150.1, 123.0, 118.7 (q, J=319.5 Hz), 107.3, 65.7, 37.7, 27.2, 23.1, 22.7, 21.9. ¹⁹F NMR (471 MHz, CDCl₃, 25° C.): 6-75.4. FTIR (thin film) cm⁻¹: 2930 (w), 1409 (s), 1245 (w), 1200 (s), 1141 (s), 977 (s). HRMS (ESI) (m/z): calc'd for C₁₁H₁₆F₃O₅S [M+H]⁺: 317.0665, found: 317.0664.

### Example 9: Synthesis of 1,4-dioxaspiro[4.7]dodec-6-yne (5)

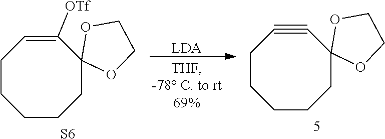

A round-bottom flask was sequentially charged with ketal S6 (70.1 mg, 222 μmol) and THE (4 mL) then cooled to −78° C. A solution of lithium diisopropylamide (LDA, 2 M in THF/heptane/ethylbenzene, 222 μL, 443 μmol) was added to the solution via syringe. The dry ice bath was immediately removed, and the solution was allowed to warm to room temperature. After 2.5 hours, the solution was cooled to −78° C. and additional lithium diisopropylamide solution (2 M in THF/heptane/ethylbenzene, 111 μL, 222 μmol) was added via syringe. The dry ice bath was removed, and the solution was allowed to warm to room temperature. After 1.5 hours, the reaction was quenched with MeOH (1.0 mL) and concentrated under reduced pressure. The crude mixture was purified by flash column chromatography on silica gel (eluent: 5% ethyl acetate in hexanes) to provide cyclooctyne 5 (25.5 mg, 69%) as a clear thin film. ¹H NMR (500 MHz, CDCl₃, 25° C.): δ 3.98-3.84 (m, 4H), 2.22 (t, J=6.4 Hz, 2H), 2.18-2.11 (m, 2H), 1.95-1.87 (m, 2H), 1.77-1.71 (m, 2H), 1.68-1.61 (m, 2H). ¹³C NMR (126 MHz, CDCl₃, 25° C.): δ 107.4, 105.3, 89.8, 64.7, 47.4, 34.2, 29.8, 27.0, 20.6. FTIR (thin film) cm⁻¹: 2926 (m), 2214 (w), 1446 (w) 1275 (w), 1170 (m), 1129 (s), 1029 (s). HRMS (ESI) (m/z): calc'd for C₁₀H₁₅O₂[M+H]⁺: 167.1067, found: 167.1067. TLC (5% ethyl acetate in hexanes) Rf: 0.38 (KMnO₄).

### Example 10: Synthesis of cyclooct-2-yn-1-yl acetate (6)

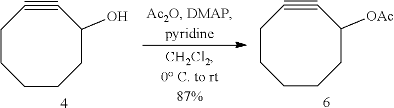

A round-bottom flask was sequentially charged with cyclooctynol 4 (80.5 mg, 648 μmol), 4-dimethylaminopyridine (6.3 mg, 51.9 μmol), and DCM (3.0 mL) at room temperature. The solution was then cooled to 0° C. with an ice-water bath, and pyridine (261 μL, 3.24 mmol) was added dropwise to the solution. Acetic anhydride (73.5 μL, 778 μmol) was then added dropwise to the solution. The ice bath was removed, and the solution was allowed to warm to room temperature. After 16 hours, the reaction was quenched with saturated aqueous ammonium chloride (1 mL) and diluted with DCM (50 mL). The organic layer was washed with water (50 mL), dried over anhydrous magnesium sulfate, and concentrated under reduced pressure. The crude mixture was purified by flash column chromatography on silica gel (eluent: 50% DCM in hexanes) to provide cyclooctyne 6 (95.5 mg, 87%) as a clear, colorless oil. ¹H NMR (500 MHz, CDCl₃, 25° C.): δ 5.34-5.26 (m, 1H), 2.31-2.21 (m, 1H), 2.21-2.08 (m, 2H), 2.02 (s, 3H), 2.02-1.93 (m, 1H), 1.94-1.83 (m, 2H), 1.83-1.72 (m, 1H), 1.72-1.57 (m, 2H), 1.57-1.46 (m, 1H). ¹³C NMR (126 MHz, CDCl₃, 25° C.): δ 170.4, 102.0, 90.8, 66.7, 41.7, 34.4, 29.8, 26.4, 21.3, 20.9. FTIR (thin film) cm⁻¹: 2930 (m), 1737 (s), 1450 (w), 1230 (s), 1025 (m), 969 (m). HRMS (ESI) (m/z): calc'd for C₁₀H₁₅O₂[M+H]⁺: 167.1067, found: 167.1068. TLC (100% CH₂Cl₂), Rf: 0.57 (I₂).

### Example 11: Synthesis of 3-fluorocyclooct-1-yne (7)

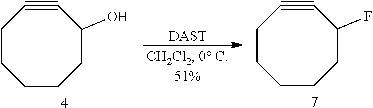

A round-bottom flask was sequentially charged with cyclooctynol 4 (40.8 mg, 329 μmol) and DCM (3.0 mL) then cooled to 0° C. Diethylaminosulfur trifluoride (DAST, 45.6 μL, 345 μmol) was then added to the solution via syringe. After 1 hour, the reaction mixture was concentrated under reduced pressure. The crude mixture was purified by flash column chromatography on silica gel (eluent: 100% pentane) to provide fluorocyclooctyne 7 (21.0 mg, 51%) as a clear, colorless oil. ¹H NMR (500 MHz, CDCl₃, 25° C.): δ 5.12 (dt, J=50.5, 5.1 Hz, 1H), 2.34-2.01 (m, 4H), 1.94-1.86 (m, 2H), 1.82-1.68 (m, 2H), 1.64-1.44 (m, 2H). ¹³C NMR (126 MHz, CDCl₃, 25° C.): δ 104.9 (d, J=10.5 Hz), 90.5 (d, J=30.0 Hz), 84.7 (d, J=171.2 Hz), 43.0 (d, J=22.9 Hz), 34.3 (d, J=1.9 Hz), 29.6, 25.5 (d, J=2.9 Hz), 20.9 (d, J=2.9 Hz). ¹⁹F NMR (471 MHz, CDCl₃, 25° C.): 6-172.2. FTIR (thin film) cm⁻¹: 2930 (s), 2855 (m), 2214 (w), 1450 (m), 1353 (m), 1029 (m), 988 (s). HRMS (ESI) (m/z): calc'd for C₈H₁₂F [M+H]⁺: 127.0918, found: 127.0916. TLC (100% pentane), Rf: 0.26 (KMnO₄).

### Example 12: Synthesis of cyclooct-2-yn-1-yl (4-nitrophenyl)carbamate (9)

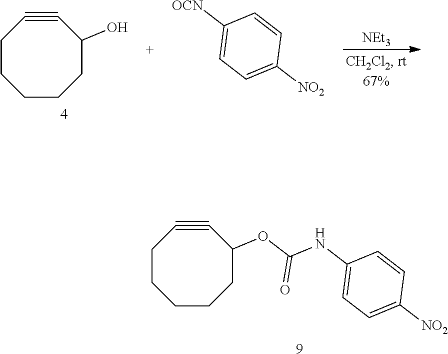

A round-bottom flask was sequentially charged with cyclooctynol 4 (10.8 mg, 87.0 μmol) and DCM (1 mL). 1-Isocyanato-4-nitrobenzene (14.3 mg, 87.0 μmol) and triethylamine (1.2 μL, 8.70 μmol) were added to the solution at room temperature. After 100 minutes, the reaction mixture was diluted with hexanes. The crude mixture was purified by flash column chromatography on silica gel (eluent: 30% diethyl ether in hexanes) to provide carbamate 9 (16.7 mg, 67%) as a white solid. ¹H NMR (500 MHz, CD₃CN, 25° C.): δ 8.26 (s, 1H), 8.16 (d, J=9.3 Hz, 2H), 7.62 (d, J=9.3 Hz, 2H), 5.32 (tq, J=5.1, 2.2 Hz, 1H), 2.35-2.24 (m, 1H), 2.24-2.15 (m, 2H), 2.08-1.99 (m, 1H), 1.94-1.86 (m, 2H), 1.86-1.75 (m, 1H), 1.73-1.63 (m, 2H), 1.64-1.52 (m, 1H). ¹³C NMR (126 MHz, CD₃CN, 25° C.): δ 153.5, 146.1, 143.8, 126.0, 118.8, 103.2, 91.6, 68.7, 42.5, 35.0, 30.4, 26.9, 21.1. FTIR (thin film) cm⁻¹: 3321 (m), 2930 (m), 1722 (s), 1566 (s), 1510 (s), 1327 (s), 1226 (s), 1055 (s). HRMS (ESI) (m/z): calc'd for C₁₅H₁₇N₂O₄ [M+H]⁺: 289.1183, found: 289.1188. TLC (50% DCM in hexanes), Rf: 0.23 (KMnO₄).

### Example 13: Synthesis of (E)-N,N-diethylcyclooct-1-en-1-amine oxide (S7)

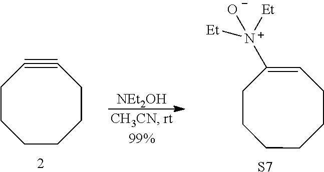 [SMILES: C1#CCCCCCC1 ; CC[N+]([O-])(CC)/C1=C/CCCCCC1 ; "S7"]

N,N-Diethylhydroxylamine (28.4 μL, 276 μmol) was added via syringe to a solution of cyclooctyne 2 (Fairbanks et al., Macromolecules 43(9):4113-4119 (2010)) (19.9 mg, 184 μmol) in acetonitrile (1.8 mL) at room temperature. After 10 minutes, the reaction mixture was concentrated under reduced pressure. The crude mixture was purified by flash column chromatography on silica gel (eluent: 15→30% CMA in chloroform) to provide enamine N-oxide S7 (36.0 mg, 99%) as a clear thin film. ¹H NMR (500 MHz, CDCl₃, 25° C.): δ 6.65 (t, J=8.8 Hz, 1H), 3.40-3.09 (m, 4H), 2.41-2.27 (m, 2H), 2.19-2.09 (m, 2H), 1.67-1.39 (m, 8H), 1.16 (t, J=7.1 Hz, 6H). ¹³C NMR (126 MHz, CDCl₃, 25° C.): δ 146.8, 125.6, 61.8, 29.7, 28.3, 26.1, 26.0, 25.6, 25.3, 8.8. FTIR (thin film) cm⁻¹: 3340 (br), 2926 (s), 2855 (m), 1655 (w), 1466 (m), 956 (s). HRMS (ESI) (m/z): calc'd for C₁₂H₂₄NO [M+H]⁺: 198.1852, found: 198.1853. TLC (50% CMA in chloroform), Rf: 0.38 (KMnO₄).

### Example 14: Synthesis of (1R,8S,9S,E)-N,N-diethyl-9-(hydroxymethyl)bicyclo[6.1.0]non-4-en-4-amine oxide (S8)

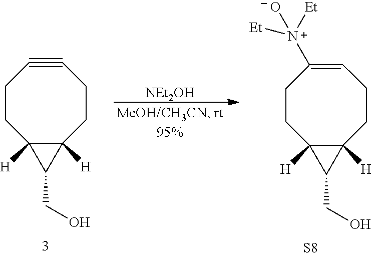 [SMILES: OC[C@H]1[C@H]2CCC#CCC[C@@H]12 ; CC[N+]([O-])(CC)/C1=C/CC[C@@H]2[C@H](CO)[C@@H]2CC1 ; "S8"]

N,N-Diethylhydroxylamine (30.8 μL, 300 μmol) was added via syringe to a solution of cyclooctyne 3 (30.0 mg, 200 μmol) in MeOH (500 μL) and acetonitrile (2.0 mL) at room temperature. After 10 minutes, the reaction mixture was concentrated under reduced pressure. The crude mixture was purified by flash column chromatography on silica gel (eluent: 20-40% CMA in chloroform) to provide enamine N-oxide S8 (45.6 mg, 95%) as a clear, colorless oil. ¹H NMR (500 MHz, CD₃OD, 25° C.): δ 6.65 (t, J=8.4 Hz, 1H), 3.72-3.60 (m, 2H), 3.61-3.42 (m, 2H), 3.40-3.29 (m, 2H), 2.63 (dt, J=16.5, 5.9 Hz, 1H), 2.55-2.40 (m, 2H), 2.26-2.12 (m, 2H), 2.15-2.02 (m, 1H), 1.74-1.57 (m, 2H), 1.21 (td, J=7.1, 2.5 Hz, 6H), 1.19-1.10 (m, 1H), 1.11-1.01 (m, 2H). 13C NMR (126 MHz, CD₃OD, 25° C.): δ 148.4, 127.5, 62.8, 62.7, 59.7, 27.4, 25.9, 24.9, 24.7, 22.6, 20.9, 19.5, 8.9, 8.8. FTIR (thin film) cm⁻¹: 3235 (br), 2986 (m), 2937 (m), 2866 (m), 1461 (m), 1375 (m), 1033 (s). HRMS (ESI) (m/z): calc'd for C₁₄H₂₆NO₂ [M+H]⁺: 240.1958, found: 240.1957. TLC (50% CMA in chloroform), Rf: 0.16 (KMnO₄).

### Example 15: Synthesis of (E)-N,N-diethyl-3-hydroxycyclooct-1-en-1-amine oxide (S9)

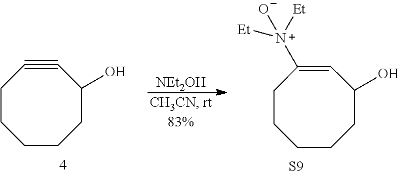 [SMILES: OC1C#CCCCCC1 ; CC[N+]([O-])(CC)/C1=C/C(O)CCCCC1 ; "S9"]

N,N-Diethylhydroxylamine (30.8 μL, 300 μmol) was added via syringe to a solution of cyclooctyne 3 (24.8 mg, 200 μmol) in acetonitrile (2.0 mL) at room temperature. After 5 minutes, the reaction mixture was concentrated under reduced pressure. The crude mixture was purified by flash column chromatography on silica gel (eluent: 20→40% CMA in chloroform) to provide enamine N-oxide S9 (35.4 mg, 83%) as a clear, colorless oil. ¹H NMR (500 MHz, CD₃OD, 25° C.): δ 6.40 (d, J=7.3 Hz, 1H), 4.55-4.44 (m, 1H), 3.66-3.55 (m, 1H), 3.53-3.42 (m, 1H), 3.41-3.31 (m, 2H), 2.58-2.48 (m, 1H), 2.48-2.36 (m, 1H), 2.00-1.92 (m, 1H), 1.88-1.76 (m, 1H), 1.76-1.43 (m, 6H), 1.31 (t, J=7.1 Hz, 3H), 1.18 (t, J=7.1 Hz, 3H). ¹³C NMR (126 MHz, CD₃OD, 25° C.): δ 146.1, 132.0, 69.9, 63.5, 61.8, 39.0, 30.9, 27.4, 27.0, 25.1, 9.1, 9.0. FTIR (thin film) cm⁻¹: 3310 (br), 2930 (s), 2490 (br), 2065 (w), 1454 (s), 1062 (s), 984 (s). HRMS (ESI) (m/z): calc'd for C₁₂H₂₄NO₂ [M+H]⁺: 214.1802, found: 214.1801. TLC (50% CMA in CHCl₃), Rf: 0.14 (KMnO₄).

### Example 16: Synthesis of (E)-N,N-diethyl-1,4-dioxaspiro[4.7]dodec-6-en-7-amine oxide (S10)

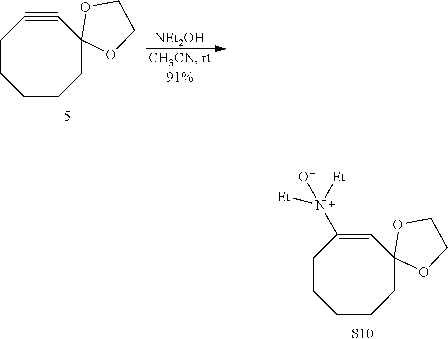 [SMILES: C1#CC2(CCCCC1)OCCO2 ; CC[N+]([O-])(CC)/C1=C/C2(CCCCC1)OCCO2 ; "S10"]

N,N-Diethylhydroxylamine (17.0 μL, 165 μmol) was added via syringe to a solution of cyclooctyne 7 (18.3 mg, 110 μmol) in acetonitrile (1.0 mL) at room temperature. After 10 minutes, the reaction mixture was concentrated under reduced pressure. The crude mixture was purified by flash column chromatography on silica gel (eluent: 15-30% CMA in chloroform) to provide enamine N-oxide S10 (25.6 mg, 91%) as a clear thin film. ¹H NMR (500 MHz, CD₃OD, 25° C.): δ 6.67 (s, 1H), 3.99-3.88 (m, 4H), 3.55-3.33 (m, 4H), 2.78 (t, J=6.7 Hz, 2H), 2.01-1.87 (m, 2H), 1.80-1.61 (m, 6H), 1.21 (t, J=7.1 Hz, 6H). ¹³C NMR (126 MHz, CD₃OD, 25° C.): δ 148.9, 131.7, 110.0, 65.4, 63.1, 40.6, 29.9, 26.0, 24.1, 23.6, 9.0. FTIR (thin film) cm⁻¹: 3355 (br), 2933 (m), 1677 (w), 1454 (m), 1081 (s), 1029 (s), 954 (s). HRMS (ESI) (m/z): calc'd for C₁₄H₂₆NO₃ [M+H]⁺: 256.1907, found: 256.1906. TLC (50% CMA in chloroform), Rf: 0.35 (KMnO₄).

### Example 17: Synthesis of (E)-3-acetoxy-N,N-diethylcyclooct-1-en-1-amine oxide (S11l)

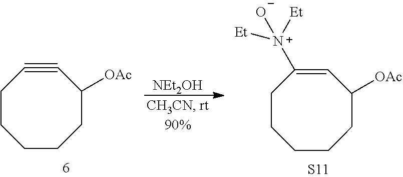 [SMILES: CC(=O)OC1C#CCCCCC1 ; CC[N+]([O-])(CC)/C1=C/C(OC(C)=O)CCCCC1 ; "S11"]

N,N-Diethylhydroxylamine (30.8 μL, 300 μmol) was added via syringe to a solution of cyclooctyne 8 (33.2 mg, 200 μmol) in acetonitrile (2.0 mL) at room temperature. After 5 minutes, the reaction mixture was concentrated under reduced pressure. The crude mixture was purified by flash column chromatography on silica gel (eluent: 10-30% CMA in chloroform) to provide enamine N-oxide S11 (46.1 mg, 90%) as a clear, colorless oil. ¹H NMR (500 MHz, CD₃OD, 25° C.): δ 6.40 (d, J=7.8 Hz, 1H), 5.47 (ddd, J=11.7, 7.7, 4.8 Hz, 1H), 3.62 (dq, J=12.5, 7.1 Hz, 1H), 3.48 (dq, J=12.4, 7.2 Hz, 1H), 3.42-3.28 (m, 2H), 2.58-2.45 (m, 2H), 2.05 (s, 3H), 2.02-1.93 (m, 1H), 1.93-1.84 (m, 1H), 1.82-1.68 (m, 3H), 1.68-1.60 (m, 1H), 1.60-1.46 (m, 2H), 1.31 (t, J=7.1 Hz, 3H), 1.12 (t, J=7.1 Hz, 3H). ¹³C NMR (126 MHz, CD₃OD, 25° C.): δ 172.2, 147.8, 128.4, 73.2, 63.6, 62.1, 35.3, 30.6, 27.3, 27.3, 24.6, 21.1, 9.1, 8.8. FTIR (thin film) cm⁻¹: 3235 (br), 2933 (w), 1730 (m), 1454 (w), 1368 (w), 1238 (s), 1029 (m). HRMS (ESI) (m/z): calc'd for C₁₄H₂₆NO₃ [M+H]⁺: 256.1907, found: 256.1905. TLC (50% CMA in chlorofrom), Rf: 0.16 (KMnO₄).

### Example 18: Synthesis of (E)-N,N-diethyl-3-fluorocyclooct-1-en-1-amine oxide (S12)

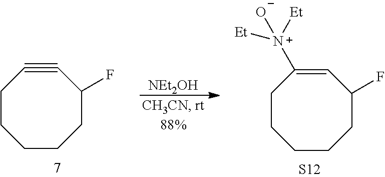 [SMILES: FC1C#CCCCCC1 ; CC[N+]([O-])(CC)/C1=C/C(F)CCCCC1 ; "S12"]

N,N-Diethylhydroxylamine (6.72 μL, 65.4 μmol) was added via syringe to a solution of cyclooctyne 7 (5.5 mg, 43.6 μmol) in acetonitrile (500 μL) at room temperature. After 10 minutes, the reaction mixture was concentrated under reduced pressure. The crude mixture was purified by flash column chromatography on silica gel (eluent: 15→30% CMA in chloroform) to provide enamine N-oxide S12 (8.3 mg, 88%) as a clear thin film. ¹H NMR (500 MHz, CD₃OD, 25° C.): δ 6.64 (dd, J=19.8, 6.5 Hz, 1H), 5.47-5.29 (m, 1H), 3.69-3.44 (m, 2H), 3.43-3.32 (m, 2H), 2.60-2.49 (m, 1H), 2.48-2.36 (m, 1H), 2.20-2.06 (m, 1H), 1.85-1.75 (m, 2H), 1.73-1.55 (m, 5H), 1.30 (t, J=7.1 Hz, 3H), 1.17 (t, J=7.1 Hz, 3H). ¹³C NMR (126 MHz, CD₃OD, 25° C.): δ 147.34 (d, J=13.4 Hz), 128.83 (d, J=33.4 Hz), 92.09 (d, J=161.6 Hz), 63.69, 62.13, 36.82 (d, J=21.9 Hz), 30.47, 26.87, 26.60, 23.98 (d, J=12.9 Hz), 8.99, 8.85. ¹⁹F NMR (471 MHz, CD₃OD, 25° C.): δ −172.0. FTIR (thin film) cm⁻¹: 3373 (br), 2937 (s), 2863 (m), 1595 (m), 1454 (m), 1379 (m), 958 (s). HRMS (ESI) (m/z): calc'd for C₁₂H₂₃FNO [M+H]⁺: 216.1758, found: 216.1758. TLC (30% CMA in chloroform), Rf: 0.13 (KMnO₄).

### Example 19: Synthesis of (E)-N,N-diethyl-3-(((4-nitrophenyl)carbamoyl)oxy)cyclooct-1-en-1-amine oxide (S13)

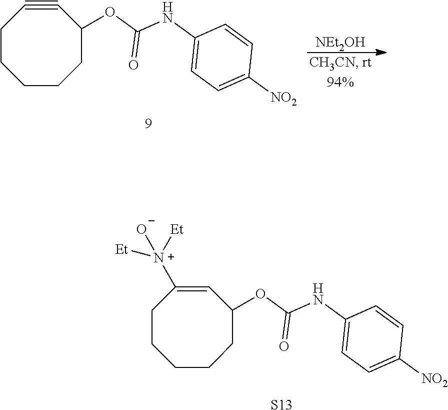 [SMILES: O=C(Nc1ccc([N+](=O)[O-])cc1)OC1C#CCCCCC1 ; CC[N+]([O-])(CC)/C1=C/C(OC(=O)Nc2ccc([N+](=O)[O-])cc2)CCCCC1 ; "S13"]

N,N-Diethylhydroxylamine (7.1 μL, 69.2 μmol) was added via syringe to a solution of cyclooctyne 9 (13.3 mg, 46.1 μmol) in acetonitrile (1.0 mL) at room temperature. After 10 minutes, the reaction mixture was concentrated under reduced pressure. The crude mixture was purified by flash column chromatography on silica gel (eluent: 30% CMA in chloroform) to provide enamine N-oxide S13 (16.3 mg, 94%) as a clear thin film. ¹H NMR (500 MHz, CD₃OD, 25° C.): δ 8.16 (d, J=9.3 Hz, 2H), 7.63 (d, J=9.3 Hz, 2H), 6.53 (d, J=7.6 Hz, 1H), 5.53 (ddd, J=11.9, 7.7, 4.9 Hz, 1H), 3.69-3.57 (m, 1H), 3.55-3.30 (m, 3H), 2.61-2.49 (m, 2H), 2.14-2.03 (m, 1H), 1.94-1.86 (m, 1H), 1.86-1.71 (m, 3H), 1.69-1.49 (m, 3H), 1.33 (t, J=7.1 Hz, 3H), 1.15 (t, J=7.2 Hz, 3H). ¹³C NMR (126 MHz, CD₃OD, 25° C.): δ 154.6, 148.0, 146.9, 143.9, 128.4, 126.0, 119.0, 74.1, 63.6, 62.2, 35.5, 30.6, 27.3, 27.3, 24.6, 9.1, 8.9. FTIR (thin film) cm⁻¹: 3198 (br), 2933 (w), 1726 (w), 1516 (m), 1327 (m), 1223 (s), 1044 (m). HRMS (ESI) (m/z): calc'd for C₁₉H₂₈N₃O₅ [M+H]⁺: 378.2023, found: 378.2021. TLC (30% CMA in chloroform), Rf: 0.26 (KMnO₄).

### Example 20: Synthesis of (E)-N,N-diethyl-3,3-difluorocyclooct-1-en-1-amine oxide

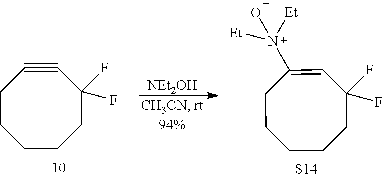

N,N-Diethylhydroxylamine (30.8 μL, 300 μmol) was added via syringe to a solution of cyclooctyne 10 (Madea et al., Chem. Commun. 52:12901-12904 (2016)) (28.8 mg, 200 μmol) in acetonitrile (1.84 mL) at room temperature. After 5 minutes, the reaction mixture was concentrated under reduced pressure. The crude mixture was purified by flash column chromatography on silica gel (eluent: 10→30% CMA in chloroform) to provide enamine N-oxide S14 (43.8 mg, 94%) as a clear thin film. ¹H NMR (500 MHz, CD₃OD, 25° C.): δ 7.02 (t, J=11.7 Hz, 1H), 3.61-3.50 (m, 2H), 3.48-3.36 (m, 2H), 2.73 (t, J=6.9 Hz, 2H), 2.27 (tt, J=15.6, 6.5 Hz, 2H), 1.85-1.61 (m, 6H), 1.21 (t, J=7.1 Hz, 6H). ¹³C NMR (126 MHz, CD₃OD, 25° C.): δ 151.9 (t, J=11.7 Hz), 126.8 (t, J=35.0 Hz), 123.7 (t, J=233.7 Hz), 63.3, 38.2, 28.3, 24.9, 24.5, 22.2, 8.8. ¹⁹F NMR (471 MHz, CD₃OD, 25° C.): 6-83.4. FTIR (thin film) cm⁻¹: 3232 (br), 2937 (m), 1692 (m), 1457 (m), 1316 (m), 988 (s). HRMS (ESI) (m/z): calc'd for C₁₂H₂₂F₂NO [M+H]⁺: 234.1664, found: 234.1664. TLC (30% CMA in chloroform), Rf: 0.059 (KMnO₄).

### Example 21: Synthesis of tert-butyl (2-(2-hydroxy(methylamino)ethoxy)ethylcarbamate (12)

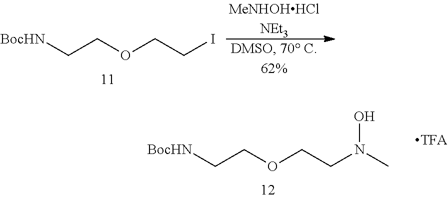 [SMILES: CC(C)(C)OC(=O)NCCOCCI ; CN(O)CCOCCNC(=O)OC(C)(C)C]

Triethylamine (1.34 mL, 9.59 mmol) was added to a solution of iodoalkane 11 (Heller et al., Angew. Chem., Int. Ed. 54(35):10327-10330 (2015)) (756 mg, 2.40 mmol) and N-methylhydroxylamine hydrochloride (401 mg, 4.80 mmol) in dimethyl sulfoxide (2.4 mL) at room temperature. The reaction mixture was then heated to 70° C. After 1.5 hours, the solution was cooled to room temperature, diluted with water, and purified by automated C₁s reverse phase column chromatography (30 g C₁s silica gel, 25 μm spherical particles, eluent: H₂O+0.1% TFA (2 CV), gradient 0→100% CH₃CN/H₂O+0.1% TFA (10 to 15 CV)) to provide hydroxylamine 12 (348 mg, 62%) as a white solid. ¹H NMR (500 MHz, CDCl₃, 25° C.) δ 3.82 (ddd, J=11.1, 7.3, 3.9 Hz, 1H), 3.63 (dt, J=11.0, 4.2 Hz, 1H), 3.52-3.35 (m, 4H), 3.32-3.18 (m, 2H), 3.07 (s, 3H), 1.38 (s, 9H). ¹³C NMR (126 MHz, CDCl₃, 25° C.) δ 164.1 (q, J=37.5 Hz), 156.7, 116.5 (q, J=289.2 Hz), 79.5, 70.9, 63.6, 60.2, 46.5, 40.4, 28.5. ¹⁹F NMR (471 MHz, CDCl₃, 25° C.) 6-75.47. FTIR (thin film) cm⁻¹: 3351 (br), 2945 (w), 2900 (w), 2236 (s), 1361 (m), 1290 (m), 1185 (s), 1129 (s), 1085 (s). HRMS (ESI) (m/z): calc'd for C₁₀H₂₃N₂O₄ [M+H]⁺: 235.1652, found: 235.1650. TLC (40% CMA in chloroform), Rf: 0.58 (I₂).

### Example 22: Synthesis of 3′,6′-bis(dimethylamino)-N-(2-(2-(hydroxy(methyl)amino)ethoxy)ethyl)-3-oxo-3H-spiro[isobenzofuran-1,9′-xanthene]-6-carboxamide (13)

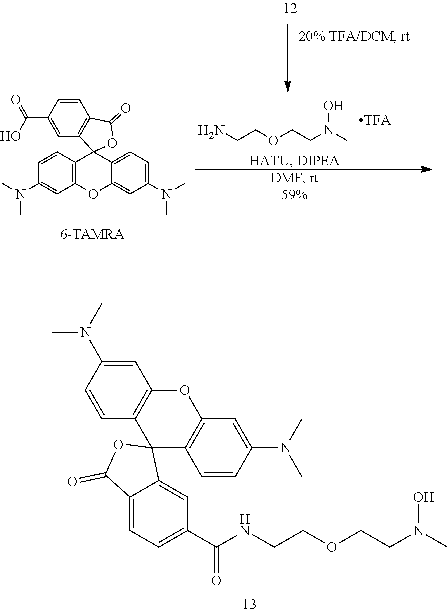

N,N-Diisopropylethylamine (DIPEA, 49.2 μL, 282 μmol) was added to a solution of 6-carboxytetramethylrhodamine (6-TAMRA, 30.4 mg, 70.6 μmol) and 1-[bis(dimethylamino)methylene]-1H-1,2,3-triazolo[4,5-b]pyridinium 3-oxid hexafluorophosphate (HATU, 29.5 mg, 77.7 μmol) in N,N-dimethylformamide (DMF, 700 μL) at room temperature. In a separate vial, trifluoroacetic acid (100 μL) was added to a solution of hydroxylamine 12 (61.5 mg, 177 μmol) in DCM (400 μL). The resulting solution stirred at room temperature for 1 hour then concentrated under reduced pressure. The resulting residue was dissolved in N,N-dimethylformamide (500 μL) then added to the reaction mixture containing 6-TAMRA using a pipette. An additional portion of N,N-dimethylformamide (200 μL) was used to quantitatively transfer the hydroxylamine solution to the reaction mixture. The reaction mixture stirred at room temperature for 4 hours. An additional portion of HATU (29.5 mg, 77.7 μmol) and DIPEA (49.2 μL, 282 μmol) was added to the reaction mixture. The solution was then stirred for another 2.5 hours. The resulting mixture was diluted with water and purified by automated C₁₈ reverse phase column chromatography (30 g C₁₈ silica gel, 25 μm spherical particles, eluent: H₂O+0.1% TFA (2 CV), gradient 0→100% CH₃CN/H₂O+0.1% TFA (10 to 15 CV)) and flash column chromatography on silica gel (eluent: 70% CMA in CHCl₃) to provide TAMRA-hydroxylamine 13 (22.8 mg, 59%) as a violet solid. ¹H NMR (500 MHz, D₂O, 25° C.) δ 7.99 (d, J=8.3 Hz, 1H), 7.87 (d, J=8.1 Hz, 1H), 7.67-7.59 (m, 1H), 7.07 (d, J=9.5 Hz, 2H), 6.73 (dd, J=9.5, 2.4 Hz, 2H), 6.41 (d, J=2.3 Hz, 2H), 3.84-3.63 (m, 4H), 3.53 (t, J=5.4 Hz, 2H), 3.11-2.89 (m, 14H), 2.71 (s, 3H). ¹³C NMR (126 MHz, D₂O, 25° C.) δ 173.2, 168.0, 157.6, 156.7, 156.6, 143.0, 133.5, 130.9, 130.6, 129.1, 128.9, 128.1, 113.6, 112.7, 96.1, 68.8, 66.6, 60.2, 47.5, 34.0, 39.7. FTIR (thin film) cm⁻¹: 3280 (br), 2926 (w), 1648 (w) 1595 (s), 1491 (m), 1409 (m), 1349 (m), 1189 (m). HRMS (ESI) (m/z): calc'd for C₃₀H₃₅N₄O₆ [M+H]⁺: 547.2551, found: 547.2544. TLC (100% CMA), Rf: 0.37 (visual).

### Example 23: Synthesis of 3-(((cyclooct-2-yn-1-yloxy)carbonyl)amino)propanoic acid

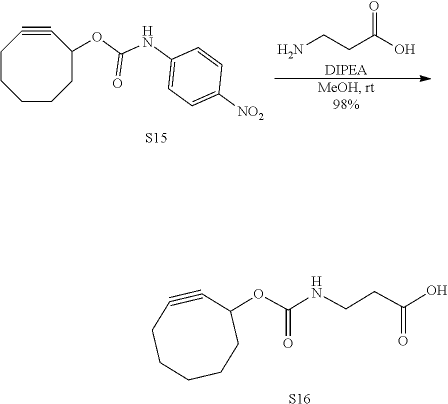 [SMILES: O=C(Nc1ccc([N+](=O)[O-])cc1)OC1C#CCCCCC1 ; NCCC(=O)O ; "S15" ; O=C(O)CCNC(=O)OC1C#CCCCCC1 ; "S16"]

3-Aminopropanoic acid (18.5 mg, 207 μmol) was added to a solution of carbonate S15 (Plass, et al., Angew. Chem., Int. Ed. 50(17):3878-3881 (2011)) (50.0 mg, 173 μmol) in MeOH (2.0 mL) at room temperature. N,N-Diisopropylethylamine (90.3 μL, 519 μmol) was then added to the solution. After 1 hour, the reaction mixture was concentrated under reduced pressure. The crude mixture was purified by flash column chromatography on silica gel (eluent: hexanes/ethyl acetate/acetic acid, v/v/v=65:30:5) to provide carbamate S16 (40.4 mg, 98%) as a clear, colorless oil. ¹H NMR (500 MHz, CD₃OD, 25° C.): δ 5.23-5.10 (m, 1H), 3.37-3.31 (m, 2H), 2.49 (t, J=6.9 Hz, 3H), 2.29-2.20 (m, 1H), 2.21-2.07 (m, 2H), 2.03-1.94 (m, 1H), 1.95-1.85 (m, 2H), 1.86-1.75 (m, 1H), 1.73-1.60 (m, 2H), 1.60-1.50 (m, 1H). ¹³C NMR (126 MHz, CD₃OD, 25° C.): δ 175.5, 158.2, 102.0, 92.4, 68.2, 43.1, 37.9, 35.5, 35.3, 31.0, 27.4, 21.3. FTIR (thin film) cm⁻¹: 2930 (m), 1700 (s), 1528 (m), 1252 (m), 1137 (w). HRMS (ESI) (m/z): calc'd for C₁₂H₁₈NO₄ [M+H]+: 240.1230, found: 240.1229. TLC (5% MeOH in DCM+0.1% acetic acid), Rf: 0.19 (I₂).

### Example 24: Synthesis of 2,5-dioxopyrrolidin-1-yl 3-(((cyclooct-2-yn-1-yloxy)carbonyl)amino)propanoate (14)

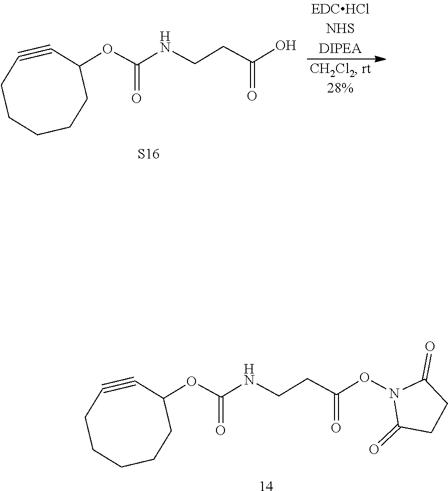 [SMILES: O=C(O)CCNC(=O)OC1C#CCCCCC1 ; "S16" ; O=C(CCNC(=O)OC1C#CCCCCC1)ON1C(=O)CCC1=O ; "14"]

N-Hydroxysuccinimide (NHS, 22.0 mg, 191 μmol), ethylcarbodiimide hydrochloride (EDC HCl, 36.7 mg, 191 μmol), and N,N-diisopropylethylamine (53.3 μL, 306 μmol) were sequentially added to a solution of carboxylic acid S16 (18.3 mg, 76.5 μmol) in DCM (1.0 mL) at room temperature. After 12 hours, additional NHS (44.0 mg, 382 μmol) and EDC HCl (73.4 mg, 382 μmol) were added to the reaction mixture. After 4 hours, the reaction mixture was concentrated under reduced pressure. The crude mixture was purified by flash column chromatography on silica gel (eluent: 30% acetone in hexanes) to provide NHS-ester 14 (7.1 mg, 28%) as a clear, colorless oil. ¹H NMR (500 MHz, CDCl₃, 25° C.): δ 5.45-5.19 (m, 2H), 3.63-3.47 (m, 2H), 2.94-2.67 (m, 6H), 2.33-2.20 (m, 1H), 2.21-2.08 (m, 2H), 2.04-1.94 (m, 1H), 1.95-1.81 (m, 2H), 1.81-1.71 (m, 1H), 1.70-1.58 (m, 2H), 1.58-1.45 (m, 2H). ¹³C NMR (126 MHz, CDCl₃, 25f° C.): δ 169.2, 167.7, 155.7, 101.9, 91.1, 67.6, 42.0, 36.6, 34.4, 32.3, 29.8, 26.4, 25.8, 20.9. FTIR (thin film) cm⁻¹: 3358 (w), 2930 (w), 1782 (w), 1733 (s), 1517 (m), 1245 (m), 1200 (s). HRMS (ESI) (m/z): calc'd for C₁₆H₂₁N₂O₆ [M+H]⁺: 337.1394, found: 337.1391. TLC (50% ethyl acetate in hexanes), Rf: 0.25 (I₂).

### Example 25: Kinetics Studies

All kinetics experiments were carried out at room temperature in CD₃CN. Reactions were monitored via NMR spectroscopy using an internal standard. Second order kinetics were performed by combining cyclooctynes and N,N-diethylhydroxylamine in a 1:1 ratio. Table 1 shows the experimental conditions for each cyclooctyne. The reported errors for rate constants are based on the standard deviation of the mean for experiments performed in triplicate.

TABLE 1
Kinetic Study of Cyclooctynes

| Com- | | | Concen- | |
|---|---|---|---|---|
| pound | Method | Internal standard | tration | k₂ (M⁻¹s⁻¹) |
| 2 | ¹H NMR | 1,3,5- | 7.1 mM | 0.0325 ± 0.0004 |
| | | trimethoxybenzene | | |
| 3 | ¹H NMR | benzotrifluoride | 5.6 mM | 0.217 ± 0.006 |
| 4 | ¹H NMR | benzotrifluoride | 3.3 mM | 1.19 ± 0.15 |
| 5 | ¹H NMR | 1,3,5- | 6.5 mM | 1.20 ± 0.09 |
| | | trimethoxybenzene | | |
| 6 | ¹H NMR | benzotrifluoride | 5.0 mM | 2.13 ± 0.03 |
| 7 | ¹⁹F NMR | benzotrifluoride | 8.0 mM | 2.71 ± 0.43 |
| 8 | ¹H NMR | 1,3,5- | 7.4 mM | 2.77 ± 0.13 |
| | | trimethoxybenzene | | |
| 9 | ¹H NMR | benzotrifluoride | 5.0 mM | 3.87 ± 0.55 |

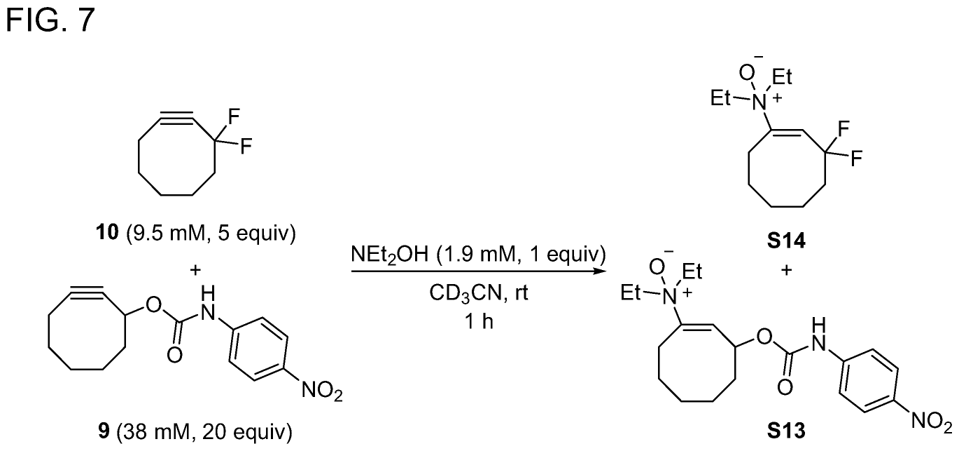

The second order rate constant for difluorocyclooctyne 10 was determined using a competition experiment with carbamate 9. N,N-diethylhydroxylamine (1 equiv; 1.9 mM final concentration) was added to a solution containing a 1:4 ratio of difluorocyclooctyne 10 (5 equiv; 9.5 mM final concentration) and cyclooctyne carbamate 9 (20 equiv; 38 mM final concentration) in CD₃CN at room temperature (FIG. 7). The solution was transferred to an NMR tube and the product ratio (S14:S13) was determined by ¹HNMR spectroscopy using 1,3,5-trimethoxylbenzene as an internal standard. The second order rate constant (k₂) of difluorocyclooctyne 10 was calculated by multiplying the observed product ratio with the second order rate constant (k₂) of carbamate 9 to give 83.6±14.9 M-'s⁻¹. The reported error for the rate constant is the standard deviation of the mean for experiments performed in triplicate.

### Example 26: Protein Labeling Experiments

Synthesis of lysozyme-COT 15

Lysozyme (CAS 12650-88-3, 50 mg/mL in deionized H₂O) was diluted into phosphate-buffered saline (PBS, pH 7.4) to a final concentration of 10 mg/mL. A solution of cyclooctyne NHS-ester 13 (65 μL, 8.5 mM in DMSO) and DMSO (10 μL) were added to the lysozyme solution (250 μL, 10 mg/mL). The reaction solution was incubated for 1 hour at room temperature. Excess cyclooctyne NHS-ester 13 was removed by spin filtration (3 kDa MWCO, 5×1:5 dilution). The concentration of lysozyme was determined by A₂₈₀ measurement in denaturing buffer (pH 7.0, 6 M guanidinium, 30 mM MOPS) on a UV-vis spectrophotometer. The solution was diluted with PBS (pH 7.4) to a final concentration of 0.15 mg/mL or 0.60 mg/mL for labeling experiments. The protein solution were snap frozen under liquid nitrogen and stored at −20° C.

Concentration-dependent protein labeling experiments

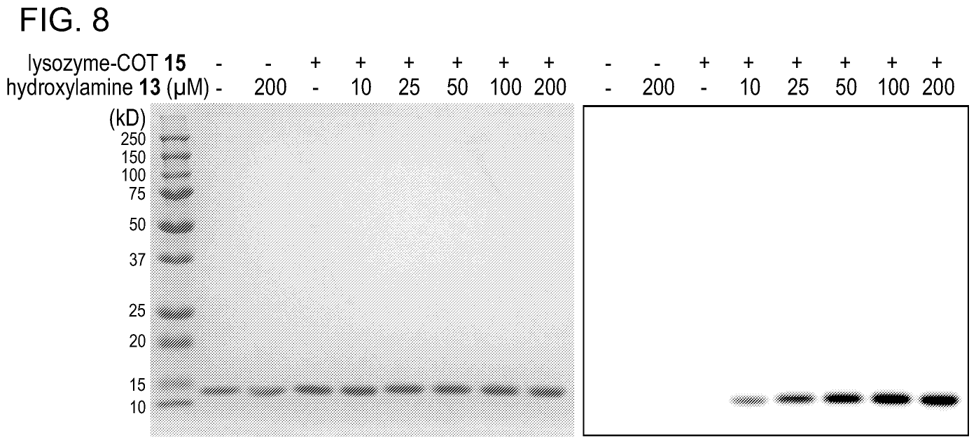

A solution of lysozyme-COT 15 (5.0 μL, 0.15 mg/mL) was aliquoted into 6 samples. An aqueous solution of hydroxylamine 13 (0.21 μL; 0.25, 0.625, 1.25, 2.5, and 5 mM in deionized water; final concentrations of 10, 25, 50, 100, and 200 μM) was added to each of 5 aliquoted samples. Deionized water (0.21 μL) was added to one sample instead of hydroxylamine as the vehicle control. Unmodified lysozyme was treated with hydroxylamine 13 (0.21 μL, 5 mM in water; 200 μM final concentration) or deionized water (0.21 μL) in control samples requiring conditions with no lysozyme-COT 15. The reaction mixtures were incubated for 2 hours at room temperature in the dark. The reaction mixtures were quenched with 5×sodium dodecyl sulfate (SDS) sample loading buffer (1.30 μL). Each solution (5 μL) was loaded onto a 15-well 12% SDS-polyacrylamide gel electrophoresis (SDS-PAGE) gel. The gel was run at room temperature and at 175 V for 50 minutes. In-gel fluorescence was imaged with a Typhoon™ FLA 9500 (GE) at 532 nm with a photomultiplier tube (PMT) setting of 500 V. The experiment was carried out in triplicate (FIG. 8).

### Time-Dependent Protein Labeling Experiments

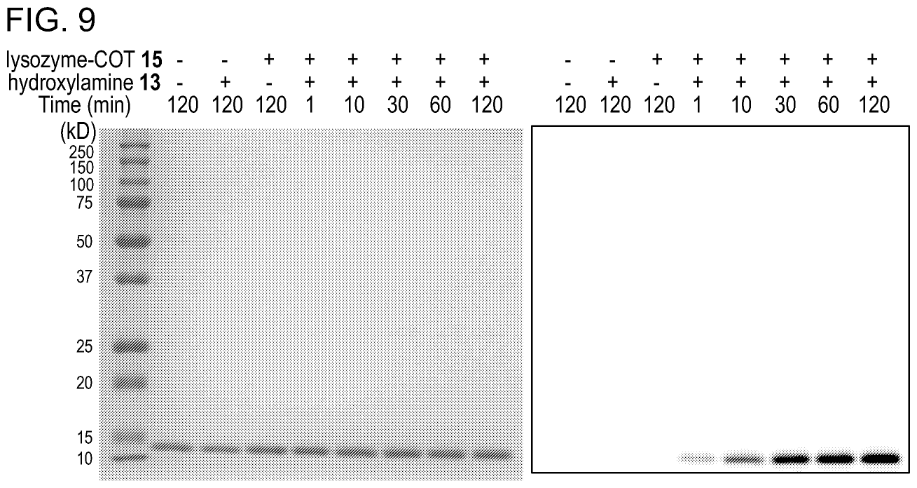

A solution of lysozyme-COT 15 (5.0 μL, 0.15 mg/mL) was aliquoted each into 6 samples. An aqueous solution of hydroxylamine 13 (0.21 μL, 5 mM in deionized water; 200 μM final concentration) was added to each of 5 aliquoted samples. Deionized water (0.21 μL) was added instead of hydroxylamine 13 to one sample for the vehicle control. Unmodified lysozyme was treated with hydroxylamine 13 (0.21 μL, 5 mM in water; 200 μM final concentration) or deionized water (0.21 μL) in control samples requiring conditions with no lysozyme-COT 15. The reaction mixtures were incubated at room temperature in the dark and quenched by adding N,N-diethylhydroxylamine (1.30 μL, 100 mM in deionized H₂O; 20 mM final concentration) followed by 5×SDS sample loading buffer (1.63 uL) at each indicated time point. Samples were snap frozen under liquid nitrogen until all samples were ready to be loaded on the gel. After 2 hours, all reactions had been quenched. All samples were thawed and each solution (5 μL) was loaded onto a 15-well 12% SDS-PAGE gel. The gel was run at room temperature and at 175 V for 50 minutes. In-gel fluorescence was imaged with a Typhoon™ FLA 9500 (GE) at 532 nm with a photomultiplier tube (PMT) setting of 500 V. The experiment was carried out in triplicate (FIG. 9).

### Intact Mass Spectrometry Analysis

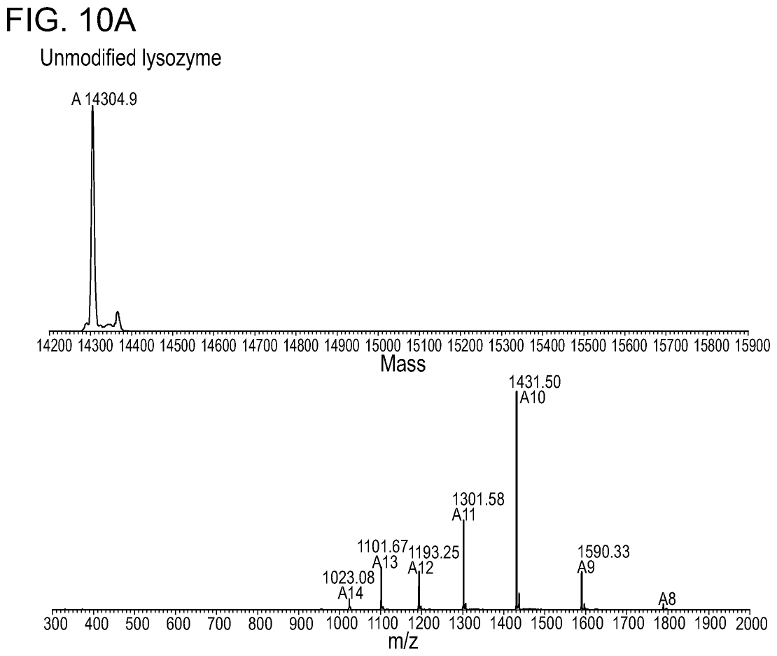

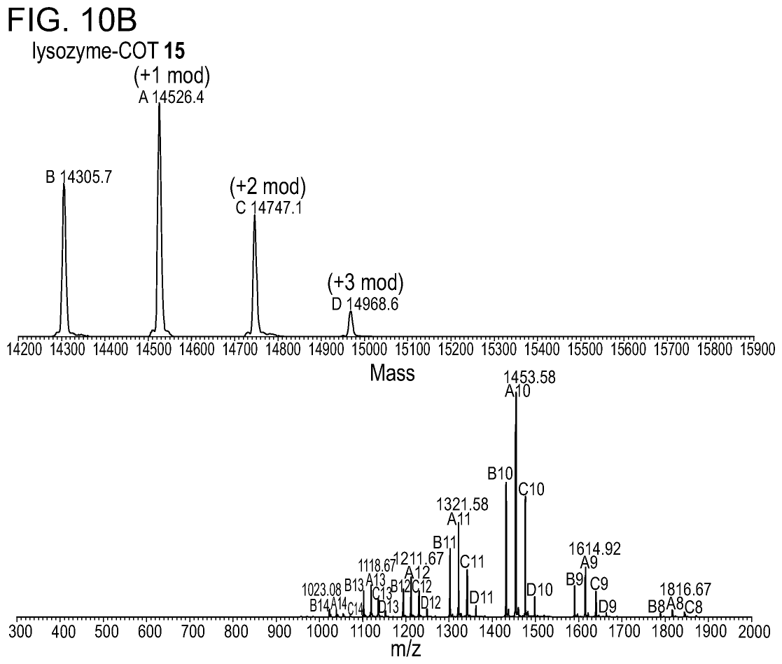

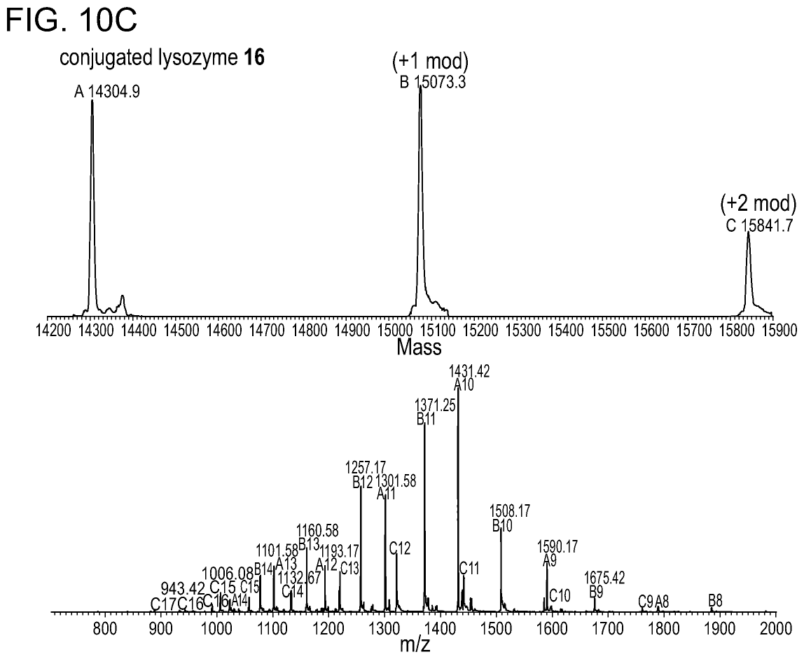

A solution of hydroxylamine 13 (0.83 μL, 5 mM in deionized water) was added to a solution of lysozyme-COT 15 (20 μL, 0.60 mg/mL in deionized water) to generate the reaction sample. Deionized H₂O (0.83 μL) was added to lysozyme-COT 15 (20 μL, 0.60 mg/mL in deionized water) to generate the vehicle control. Unmodified lysozyme (20 μL, 0.60 mg/mL in deionized water) was added to deionized water (0.83 μL) to generate the blank background sample. Reactions were incubated at room temperature for 6 hours in the dark. The samples were snap frozen using liquid nitrogen and stored at −80° C. until further analysis. Electrospray ionization mass spectrometry (ESI-MS) analysis was performed on an LTQ XL™ ion trap mass spectrometer (ThermoFisher Scientific™, San Jose, CA) (FIG. 10).

### Example 27: Protein Labeling Experiments in the Presence of Cell Lysate

Cell Culture: HEK-293T cells were cultured in Dulbecco's Modified Eagle Media (DMEM, Corning®) containing 10% fetal bovine serum (FBS, Sigma), 100 units/mL penicillin, and 0.1 mg/mL streptomycin (Sigma) in a humidified chamber at 37° C. with 5% CO₂. Cells were passaged and dissociated with 0.25% trypsin, 0.1% ethylenediaminetetraacetic acid (EDTA) in Hanks' balanced salt solution (HBSS) (Corning®). Cells tested negative for mycobacteria by the MycoAlert™ PLUS Mycoplasma Detection Kit (Lonza) following the manufacturer's protocol.

Cell Lysate: Cell culture media was aspirated prior to lysis. Cells (10 cm dish, ˜80% confluency) was lysed by adding lysis buffer (1.0 mL, 4° C.; 150 mM NaCl, 50 mM Tris (pH 8.0), 1% triton X-100). After centrifugation (13,000×g) at 4° C., the supernatant was transferred to a clean tube, and the protein concentration was determined by BCA (bicinchoninic acid) protein assay (Pierce™ BCA Protein Assay Kit). Aliquots of the cell lysate (7.1 mg/mL) were snap frozen under liquid nitrogen and stored at −20° C.

Protein Labeling Experiments: Lysozyme-COT 15 (0.75 μg, 5.0 μL, 0.15 mg/mL in deionized water) was aliquoted to form 4 samples. Cell lysate (20 μg, 2.83 μL, 7.1 mg/mL) was added to 2 samples, and deionized water (2.83 μL) was added to the remaining 2 samples. A fifth control sample lacking lysozyme-COT 15 was prepared by adding cell lysate (20 μg, 2.83 μL, 7.1 mg/mL) to deionized water (5.0 μL). Then, either hydroxylamine 13 (0.33 μL, 5 mM in deionized water) or deionized water (0.33 μL) were added to the samples according to the conditions laid out in FIG. 5B. The reaction mixtures were incubated for 2 hours at room temperature in the dark. The reaction mixtures were quenched with 5×SDS sample loading buffer (1.30 uL). Each solution (5 μL) was loaded onto a 15-well 12% SDS-PAGE gel. The gel was run at room temperature and at 175 V for 50 min. In-gel fluorescence was imaged with a Typhoon™ FLA 9500 (GE) at 532 nm with a photomultiplier tube (PMT) setting of 500 V. The experiment was carried out in triplicate.

### Example 28: Stability Studies

All reactions were monitored by HPLC at 0, 1, 2, 4, 8, and 24 hour time points.

HPLC analysis: Reactions with enamine N-oxide 17 were analyzed by HPLC (Pursuit 200 Å C₁₈, 4.6×150 mm, 10 μm particles, 1 mL/min flow rate, eluent: isocratic 0% MeCN/H₂O+0.1% TFA (1 min), gradient 0→100% MeCN/H₂O+0.1% TFA (14 minutes), isocratic 100% MeCN/H₂O+0.1% TFA (1 minute)) and quantified using its absorbance at 280 nm. Reactions with hydroxylamine 13 were analyzed by HPLC (Pursuit 200 Å C₁₈, 4.6×150 mm, 10 μm particles, 1 mL/min flow rate, eluent: isocratic 0% MeCN/H₂O+0.1% TFA (1 minute), gradient 0-20% MeCN/H₂O+0.1% TFA (1 minute), gradient 20-50% MeCN/H₂O+0.1% TFA (16 minutes), gradient 50→100% MeCN/H₂O+0.1% TFA (2 minutes), isocratic 100% MeCN/H₂O+0.1% TFA (1 minute)) and quantified using its absorbance at 254 nm.

Stability in PBS: Enamine N-oxide 17 (4 μL, 15 mM in 25% v/v MeOH/PBS, pH 7.4) was added to PBS (116 μL, pH 7.4; 500 μM final concentration). Separately, hydroxylamine 13 (3 μL, 20 mM in deionized H₂O) was added to PBS (117 μL, pH 7.4; 500 μM final concentration). Each solution was monitored by HPLC at each time point.

Stability in the presence of glutathione, sodium ascorbate, and cell lysate: The reactions were conducted as described above in the Stability in PBS section except the PBS solution was first supplemented with glutathione (5 mM final concentration), sodium ascorbate (5 mM final concentration), or HEK293T cell lysate (1 mg/mL final concentration) and pH adjusted to 7.4 before adding enamine N-oxide 17 (500 μM final concentration) or hydroxylamine 13 (500 μM final concentration).

Microsomal assay I: A solution of human liver microsomes (8 μL, 20 mg/mL in phosphate buffer, pH 7.4, Corning®; 200 μg/mL final concentration) and a solution of NADPH (13.4 μL, 60 mM in 10 mM NaOH solution) were sequentially added to PBS (751.9 μL, pH 7.4) in a 2.0 mL microcentrifuge tube. The solution was incubated for 1 hour at room temperature to provide solution A. A solution of enamine N-oxide 17 (26.7 μL, 15 mM in 25% MeOH/PBS, pH 7.4; 500 μM final concentration) was added to solution A. The cap of the microcentrifuge tube was pierced with a 16G needle to maintain an aerobic system. The reaction was incubated at room temperature in the dark. At each time point, 100 μL of the sample was transferred to a clean 2.0 mL microcentrifuge tube, and the reaction was quenched with acetonitrile (100 μL). The mixture was centrifuged (13,000×g) at 4° C. for 5 minutes, then the supernatant was transferred to a clean HPLC vial for analysis.

Microsomal assay II: A solution of human liver microsomes (8 μL, 20 mg/mL in phosphate buffer, pH 7.4, Corning®; 200 μg/mL final concentration) and a solution of NADPH (13.4 μL, 60 mM in 10 mM NaOH solution; 1 mM final concentration) were sequentially added to PBS (358.6 μL, pH 7.4) and incubated at room temperature for 1 hour to provide solution B. A solution of sodium ascorbate (400 μL, 10 mM in PBS, pH 7.4) and a solution of hydroxylamine 13 (20 μL, 20 mM in deionized H₂O) were added to solution B. The cap of the microcentrifuge tube was pierced with a 16G needle to maintain an aerobic system. The reaction was incubated at room temperature in the dark. At each time point, 100 μL of the sample was transferred to a clean 2.0 mL microcentrifuge tube, and the reaction was quenched with acetonitrile (100 μL). The mixture was centrifuged (13,000×g) at 4° C. for 5 minutes and the supernatant was transferred to a clean HPLC vial for analysis.

### Example 29: Cross Reactivity Studies

Cyclooctynes with Me-tetrazine: A solution of cyclooctyne 5, 9, or 10 (125 μL, 20 mM in CD₃CN, 1 equiv; 5 mM final concentration) was each added to a separate NMR tube. A solution containing the internal standard 1,3,5-trimethoxybenzene (TMB, 50 μL, 50 mM in CD₃CN; 5 mM final concentration) was then added via syringe to each of the samples. CD₃CN (275 μL) was added to each tube to bring the volume of each solution to 450 μL. The tubes were inverted three times to mix the solutions, and reference spectra were obtained by ¹H NMR spectroscopy. A solution of tert-butyl-(4-(6-methyl-1,2,4,5-tetrazin-3-yl)benzyl)carbamate (18, 50 μL, 50 mM in CD₃CN, 2.00 equiv; 10 mM final concentration) was added to bring the final volume to 500 μL. The tubes were immediately inverted three times to mix the reaction solutions then incubated for 1 hour at room temperature. The reaction mixtures were analyzed by ¹H NMR spectroscopy. No change in the spectra was observed.

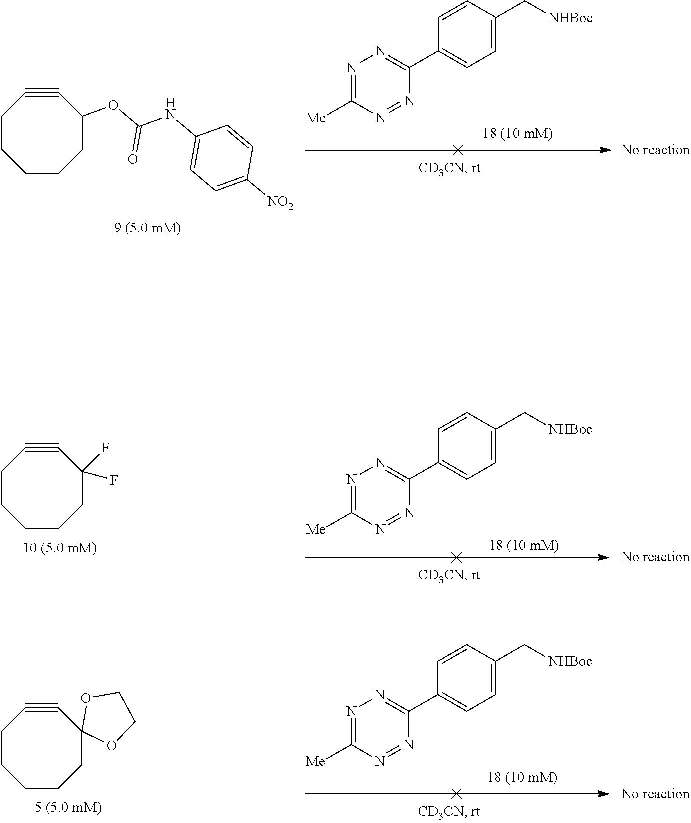 [SMILES: O=C(Nc1ccc([N+](=O)[O-])cc1)OC1C#CCCCCC1 ; Cc1nnc(-c2ccc(CNC(=O)OC(C)(C)C)cc2)nn1 ; FC1(F)C#CCCCCC1 ; Cc1nnc(-c2ccc(CNC(=O)OC(C)(C)C)cc2)nn1 ; C1#CC2(CCCCC1)OCCO2 ; Cc1nnc(-c2ccc(CNC(=O)OC(C)(C)C)cc2)nn1]

Hydroxylamines with various components: A solution of (E)-cyclooct-4-en-1-yl (3-aminopropyl)carbamate (19, 16 mM in 6.2% D₂O in CD₃CN; 5 mM final concentration), 4-methylbenzaldehyde (20, 100 mM in CD₃CN; 5 mM final concentration), (2-methylcycloprop-2-en-1-yl)methyl isopropylcarbamate (21, 50 mM in CD₃CN; 5 mM final concentration), or hex-5-ynoic acid (22, 50 mM in CD₃CN; 5 mM final concentration) was each added to a separate NMR tube. A solution containing the internal standard 1,3,5-trimethoxybenzene (TMB, 50 μL, 50 mM in CD₃CN; 5 mM final concentration) was then added via syringe to each of the samples. CD₃CN was added to each tube to bring the volume of each solution to 475 μL. The tubes were inverted three times to mix the solutions, and reference spectra were obtained by ¹H NMR spectroscopy. For each reaction, a solution of N,N-diethylhydroxylamine (25 μL, 100 mM in CD₃CN, 2.00 equiv; 10 mM final concentration) was added to bring the final volume to 500 μL. The tubes were immediately inverted three times to mix the reaction solutions then incubated for 1 hour at room temperature. The reaction mixtures were analyzed by ¹H NMR spectroscopy. No change in the spectra was observed.

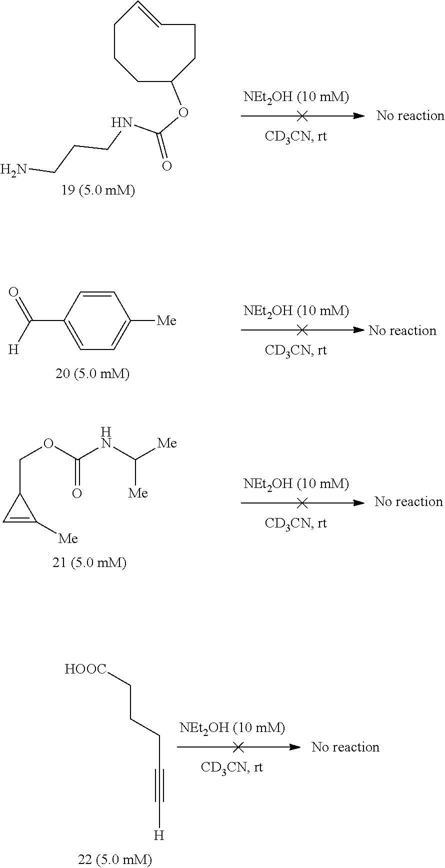 [SMILES: NCCCNC(=O)OC1CC/C=C/CCC1 ; Cc1ccc(C=O)cc1 ; CC1=CC1COC(=O)NC(C)C ; C#CCCCC(=O)O]

Cu-catalyzed azide-alkyne cycloaddition (CuAAC): A 2.0 mM microcentrifuge tube was sequentially charged with solutions of 3′,6′-bis(dimethylamino)-3-oxo-N-(prop-2-yn-1-yl)-3H-spiro[isobenzofuran-1,9′-xanthene]-5-carboxamide (S17, Click Chemistry Tools 1255-5, 8 μL, 5 mM in DMSO), 3,3′,3‘ ’-(4,4′,4‘ ’-(nitrilotris(methylene))tris(1H-1,2,3-triazole-4,1-diyl))tris(propan-1-ol) (THPTA, 0.6 μL, 100 mM in DMSO), CuSO₄·H₂O (0.4 μL, 50 mM in PBS, pH 7.4), sodium ascorbate (10 μL, 100 mM in PBS, pH 7.4), and PBS (180.2 μL, pH 7.4) at room temperature. A solution of N,N-diethylhydroxylamine (0.8 μL, 100 mM in PBS, pH 7.4) was added to this mixture to bring the total volume to 200 μL. The solution was immediately transferred to a clean HPLC vial to monitor the reaction progress and incubated at room temperature in the dark. The reaction was analyzed by HPLC (Pursuit 200 Å C₁₈, 4.6×150 mm, 10 μm particles, 1 mL/min flow rate, eluent: isocratic 0% MeCN/H₂O+0.1% TFA (1 minute), gradient 0→100% MeCN/H₂O+0.1% TFA (14 minutes), isocratic 100% MeCN/H₂O+0.1% TFA (1 minute)) at each time point (0, 1, 2, and 4 hours) and quantified using its absorbance at 280 nm. No reaction was observed.

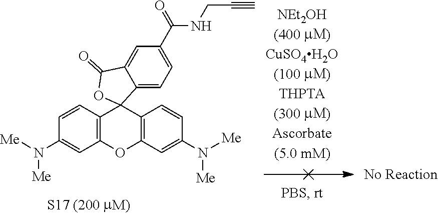 [SMILES: C#CCNC(=O)c1ccc2c(c1)C(=O)OC21c2ccc(N(C)C)cc2Oc2cc(N(C)C)ccc21]

### Example 30: Computational Details

All calculations were conducted with Gaussian 09 software (Frisch et al., Gaussian 16, Revision C.01, Gaussian, Inc., Wallingford CT, (2019)). Geometry optimization of all species was performed using the M06-2X functional (Zhao etal., Theor. Chem. Acc. 120:215-241 (2008)) with the 6-31G(d) basis set. Frequency analysis was carried out to ensure the stationary point was either a minimum or a transition state. Intrinsic reaction coordinates were computed for all transition states. Single-point calculations were carried out using the M06-2X functional with the 6-311G(2d,p) basis set. For each cyclooctyne, at least three different conformers were analyzed via geometry optimization and the most stable conformation was adapted to locate the transition states. The 3D image in FIG. 2B was generated by using CYLview (CYLview, 1.0b; Legault, C. Y., Universite de Sherbrooke, 2009 (http://www.cylview.org)).

Cartesian coordinates of optimized structures (Å)

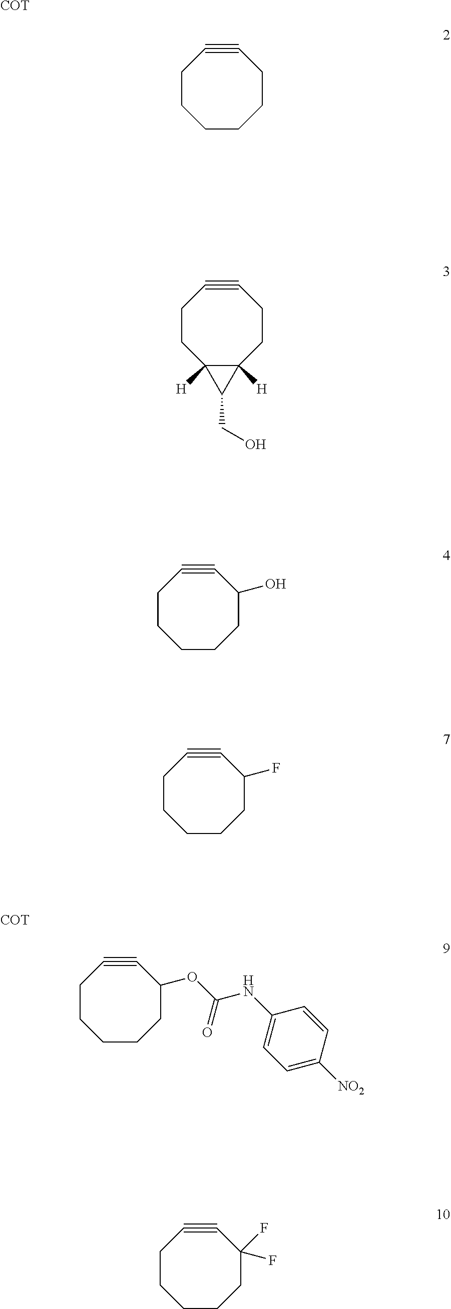

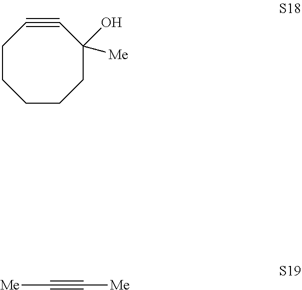

2

Lowest three frequencies (cm⁻¹): 110.03, 162.71, 241.84

E(RM062X)=−311.941223533

| | | | |
|---|---|---|---|
| C | −0.68292200 | 1.34395100 | −0.37416000 |
| C | 0.68329400 | 1.34396600 | 0.37395400 |
| C | −0.60303500 | −1.45391500 | −0.03049400 |
| C | 1.85549200 | 0.58004700 | −0.28167000 |
| C | 0.60265200 | −1.45397600 | 0.02990500 |
| C | 1.95689300 | −0.90982700 | 0.12276300 |
| H | −0.54219700 | 0.96955700 | −1.39589000 |
| H | 0.54250900 | 0.96977100 | 1.39573900 |
| H | 1.75673900 | 0.64014800 | −1.37149500 |
| H | −1.00069900 | 2.38614400 | −0.48058600 |
| H | 1.00126400 | 2.38613400 | 0.48016800 |
| H | 2.80150000 | 1.06939900 | −0.02463200 |
| H | 2.32183800 | −0.99477600 | 1.15300500 |
| C | −1.85519100 | 0.58044700 | 0.28178400 |
| H | −2.80118600 | 1.06994000 | 0.02495600 |
| H | −1.75621700 | 0.64055900 | 1.37159300 |
| C | −1.95726000 | −0.90951700 | −0.12239500 |
| H | −2.32324900 | −0.99453800 | −1.15225700 |
| H | −2.67291700 | −1.43432800 | 0.51762800 |
| H | 2.67307900 | −1.43506700 | −0.51634600 |

2-TS

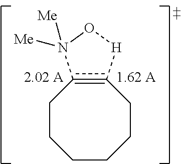

Lowest three frequencies (cm⁻¹): −210.84, 54.59, 63.58

E(RM062X)=−597.454128075

| | | | |
|---|---|---|---|
| C | −2.66445400 | −1.33159700 | 0.25384500 |
| C | −2.70860600 | 0.11134700 | 0.77854500 |
| C | 0.23405900 | −0.01687700 | −0.10192300 |
| C | −2.62269200 | 1.20627700 | −0.30455500 |
| C | −0.10056100 | 1.18988900 | −0.16109100 |
| C | −1.32770700 | 2.03024300 | −0.20765300 |
| H | −3.59000700 | −1.53272000 | −0.30043300 |
| H | −1.89403400 | 0.25541200 | 1.49723700 |
| H | −2.67496500 | 0.75304900 | −1.30180100 |
| H | −2.67237900 | −2.01222300 | 1.11501700 |
| H | −3.63774000 | 0.22238000 | 1.34753100 |
| H | −3.48153200 | 1.88284300 | −0.23173900 |
| H | −1.35646000 | 2.64314400 | 0.70289600 |
| C | −1.49155500 | −1.71260800 | −0.65786900 |
| H | −1.56283600 | −1.17719800 | −1.61096600 |
| H | −1.58407100 | −2.77893300 | −0.89383500 |
| C | −0.08708600 | −1.46293400 | −0.08011000 |
| H | 0.64290000 | −2.03036700 | −0.66907100 |
| H | −0.03127100 | −1.85344000 | 0.94630500 |
| H | −1.26789200 | 2.74388800 | −1.03715400 |
| H | 1.42967300 | 1.69564600 | −0.00213800 |
| N | 2.24740000 | 0.04409500 | 0.06739800 |
| C | 2.67445500 | −0.55729400 | 1.32517900 |
| H | 2.47108700 | −1.63102300 | 1.29984500 |
| H | 3.74417800 | −0.38817100 | 1.49393400 |
| H | 2.10026100 | −0.09070700 | 2.12625200 |
| C | 2.91198200 | −0.49989900 | −1.11211100 |
| H | 3.98469800 | −0.27849500 | −1.08307300 |
| H | 2.76495000 | −1.58165900 | −1.15333500 |
| H | 2.46586200 | −0.03045300 | −1.98961400 |
| O | 2.43884600 | 1.39263400 | 0.10985100 |

3

Lowest three frequencies (cm⁻¹): 76.84, 119.98, 150.60

E(RM062X)=−464.533804304

| | | | |
|---|---|---|---|
| C | −0.01283500 | −1.00254400 | −0.80085300 |
| C | 0.37899600 | 0.45714600 | −0.91815300 |
| C | −2.50601300 | −0.06311900 | 0.26412800 |
| C | 0.17822400 | 1.53774600 | 0.13058700 |
| C | −2.17464500 | 1.09620100 | 0.18957400 |
| C | −1.22089500 | 2.19913000 | 0.04245500 |
| H | 0.29307700 | 1.12838000 | 1.13911500 |
| H | 0.96102000 | 2.29404100 | 0.00591900 |
| H | −1.33393400 | 2.96063000 | 0.82004300 |
| H | −1.34715300 | 2.70086200 | −0.92366200 |
| C | −0.72754500 | −1.66251700 | 0.36754800 |
| H | −0.40894300 | −1.23089700 | 1.32086800 |
| H | −0.46644200 | −2.72809700 | 0.39078900 |
| C | −2.26809700 | −1.50908400 | 0.28252000 |
| H | −2.74729900 | −2.00550200 | 1.13185500 |
| H | −2.65066000 | −1.98299900 | −0.62864600 |
| C | 1.44406000 | −0.60117600 | −0.78352100 |
| H | 2.02067800 | −0.81919500 | −1.67846400 |
| H | −0.33231800 | −1.42929600 | −1.75073000 |
| H | 0.26603600 | 0.84783700 | −1.92828500 |
| C | 2.27399100 | −0.66775500 | 0.46982800 |
| H | 2.81936200 | −1.62295200 | 0.49704300 |
| H | 1.63764000 | −0.63371900 | 1.36438300 |
| O | 3.16866000 | 0.43112800 | 0.44543400 |
| H | 3.74821600 | 0.36770100 | 1.21162500 |

3-TS

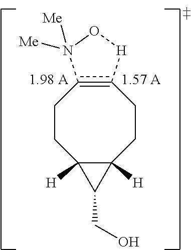

Lowest three frequencies (cm⁻¹): −258.31, 39.54, 58.07

E(RM062X)=−674.832028183

| | | | |
|---|---|---|---|
| C | 1.85584600 | 1.06409700 | 0.78212800 |
| C | 1.62694400 | −0.41961300 | 0.91973700 |
| C | −1.04885100 | 1.27280900 | −0.19321700 |
| C | 0.98383100 | −1.26256600 | −0.15990500 |
| C | −1.14074600 | 0.02091800 | −0.10622200 |
| C | −0.54380500 | −1.33232900 | −0.00964000 |
| H | 1.22044700 | −0.87404600 | −1.15497900 |
| H | 1.40065900 | −2.27447500 | −0.11594200 |
| H | −0.95309500 | −1.99803500 | −0.77924300 |
| H | −0.78749500 | −1.78685000 | 0.96036800 |
| C | 1.41279300 | 1.86504500 | −0.42404200 |
| H | 1.48590100 | 1.27450200 | −1.34237400 |
| H | 2.08124200 | 2.72700200 | −0.54972500 |
| C | −0.03588500 | 2.35807800 | −0.28617200 |
| H | −0.27924100 | 3.01464000 | −1.12973000 |
| H | −0.12165500 | 2.99189500 | 0.60659000 |
| C | 3.02875000 | 0.11200100 | 0.77360300 |
| H | 3.63480200 | 0.09044100 | 1.67524700 |
| H | 1.74518500 | 1.62357000 | 1.70963600 |
| H | 1.36736400 | −0.75253000 | 1.92312200 |
| C | 3.82436500 | −0.17914900 | −0.46937700 |
| H | 4.72265600 | 0.45584100 | −0.48417000 |
| H | 3.24564900 | 0.06069500 | −1.37137600 |
| O | 4.17246200 | −1.55384200 | −0.44345500 |
| H | 4.74174300 | −1.73555900 | −1.19835200 |
| H | −2.60870700 | 1.47160700 | −0.14425100 |
| N | −3.09562800 | −0.30309800 | −0.01817100 |
| C | −3.56241300 | −1.04259200 | −1.18780100 |
| H | −3.19063300 | −2.06896000 | −1.14779700 |
| H | −4.65713500 | −1.04908900 | −1.22039300 |
| H | −3.17289900 | −0.53892700 | −2.07311300 |
| C | −3.47059100 | −0.90818400 | 1.25669400 |
| H | −4.56019000 | −0.91712300 | 1.36816900 |
| H | −3.08753900 | −1.93024900 | 1.30381600 |
| H | −3.02642200 | −0.30672500 | 2.05058300 |
| O | −3.56154500 | 0.97296200 | −0.07324700 |

4

Lowest three frequencies (cm⁻¹): 88.35, 145.59, 170.29

E(RM062X)=−387.155738469

| | | | |
|---|---|---|---|
| C | −1.40444800 | 1.16077100 | −0.45525300 |
| C | −0.15369900 | 1.58367700 | 0.37115600 |
| C | −0.55528900 | −1.47041500 | 0.07093300 |
| C | 1.22920300 | 1.15210200 | −0.15836200 |
| C | 0.60308800 | −1.14617500 | 0.18041400 |
| C | 1.72348700 | −0.20838500 | 0.36079200 |
| H | −1.08845000 | 0.80277200 | −1.44282500 |
| H | −0.26482200 | 1.23800300 | 1.40698100 |
| H | 1.21662800 | 1.08668700 | −1.25184400 |
| H | −1.99693200 | 2.06216200 | −0.64089700 |
| H | −0.14809000 | 2.67654700 | 0.42585000 |
| H | 1.98863200 | 1.89489700 | 0.10777200 |
| H | 1.93435700 | −0.12372800 | 1.43908100 |
| C | −2.35600600 | 0.12050400 | 0.17627500 |
| H | −3.37963800 | 0.29968800 | −0.17009600 |
| H | −2.36437800 | 0.25488400 | 1.26382800 |
| C | −1.99639300 | −1.35254800 | −0.13627700 |
| H | −2.23863400 | −1.58396400 | −1.17979900 |
| O | 2.90095600 | −0.52443600 | −0.35070800 |
| H | −2.57669200 | −2.03460500 | 0.49195500 |
| H | 3.17071800 | −1.41504200 | −0.10241500 |

4-TS

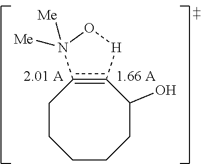

Lowest three frequencies (cm⁻¹): −182.60, 48.39, 53.20

E(RM062X)=−597.454857190

| | | | |
|---|---|---|---|
| C | 2.08255600 | −1.79648500 | 0.50416900 |
| C | 3.00686600 | −0.60652000 | 0.17522800 |
| C | −0.39646100 | −0.30639400 | −0.25881200 |
| C | 2.53293300 | 0.81360200 | 0.50680400 |
| C | 0.17386700 | 0.80649400 | −0.30114300 |
| C | 1.50508500 | 1.44094700 | −0.44640700 |
| H | 1.54248400 | −1.58854500 | 1.43749800 |
| H | 3.29050300 | −0.65447100 | −0.88525800 |
| H | 2.12658000 | 0.84187300 | 1.52703400 |
| H | 2.73013100 | −2.65278000 | 0.71845400 |
| H | 3.93735500 | −0.75727000 | 0.73362400 |
| H | 3.40093100 | 1.48157800 | 0.49285600 |
| H | 1.85519100 | 1.29840300 | −1.47741300 |
| C | 1.08112500 | −2.23005700 | −0.58633000 |
| H | 1.07999600 | −3.32178900 | −0.67386800 |
| H | 1.40009400 | −1.83936800 | −1.55966400 |
| C | −0.36287800 | −1.77946800 | −0.32834500 |
| H | −0.71887500 | −2.22049600 | 0.61249200 |
| O | 1.47488800 | 2.85025700 | −0.26426300 |
| H | −1.01750800 | −2.15439800 | −1.12448300 |
| H | 0.92877100 | 3.01758800 | 0.51218300 |
| H | −1.25869700 | 1.61496200 | −0.05276500 |
| N | −2.33096700 | 0.12430200 | 0.11897700 |
| C | −2.73729100 | −0.36679100 | 1.43113600 |
| H | −3.71547700 | 0.04094000 | 1.70898000 |
| H | −2.78670300 | −1.45794900 | 1.41167400 |
| H | −1.98711300 | −0.04247600 | 2.15367600 |
| C | −3.19730300 | −0.30361200 | −0.97438400 |
| H | −3.23588700 | −1.39513400 | −1.00105200 |
| H | −4.20854100 | 0.09728100 | −0.84384400 |
| H | −2.77091100 | 0.07593100 | −1.90368900 |
| O | −2.27570700 | 1.48920700 | 0.14541600 |

7

Lowest three frequencies (cm⁻¹): 88.56, 145.94, 170.90

E(RM062X)=−411.178599922

| | | | |
|---|---|---|---|
| C | −1.38416000 | 1.17714000 | −0.44941600 |
| C | −0.12115600 | 1.58443800 | 0.36548100 |
| C | −0.56314300 | −1.46906000 | 0.06275800 |
| C | 1.25205200 | 1.12418600 | −0.16592300 |
| C | 0.60291400 | −1.17376500 | 0.16709000 |
| C | 1.71425700 | −0.24071200 | 0.36059400 |
| H | −1.08281100 | 0.82163800 | −1.44247400 |
| H | −0.23343700 | 1.25285000 | 1.40583200 |
| H | 1.23256600 | 1.05238500 | −1.25883900 |
| H | −1.96802900 | 2.08619500 | −0.62363400 |
| H | −0.09415300 | 2.67726600 | 0.40855400 |
| H | 2.02438100 | 1.85539600 | 0.09452300 |
| H | 1.96673200 | −0.17605900 | 1.42663400 |
| C | −2.34093300 | 0.14522000 | 0.18740100 |
| H | −3.36537300 | 0.33795000 | −0.14849400 |
| H | −2.33777300 | 0.27564000 | 1.27541600 |
| C | −2.00307400 | −1.33077300 | −0.13423800 |
| H | −2.25340600 | −1.55450000 | −1.17742400 |
| H | −2.58721000 | −2.00872000 | 0.49456300 |
| F | 2.86199800 | −0.61334300 | −0.31301600 |

7-TS

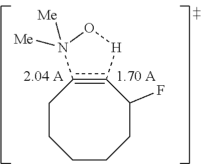

Lowest three frequencies (cm⁻¹): −148.74, 47.84, 53.77

E(RM062X)=−621.4807673

| | | | |
|---|---|---|---|
| C | 2.09957800 | −1.77336700 | 0.50460200 |
| C | 3.01413400 | −0.58224700 | 0.14693100 |
| C | −0.38454600 | −0.30961600 | −0.25940900 |
| C | 2.54526100 | 0.83632400 | 0.49176100 |
| C | 0.18142700 | 0.80059500 | −0.29535300 |
| C | 1.49617300 | 1.43441900 | −0.44107000 |
| H | 1.56875600 | −1.55399600 | 1.44030900 |
| H | 3.27029300 | −0.63251200 | −0.92038700 |
| H | 2.15375100 | 0.86941500 | 1.51495300 |
| H | 2.75601900 | −2.62099800 | 0.72545600 |
| H | 3.95914000 | −0.72898600 | 0.68068600 |
| H | 3.40828000 | 1.51022900 | 0.45742300 |
| H | 1.83260100 | 1.34662500 | −1.48390800 |
| C | 1.08899200 | −2.23525000 | −0.56665100 |
| H | 1.09042100 | −3.32878100 | −0.62654600 |
| H | 1.39955200 | −1.86880400 | −1.55210600 |
| C | −0.35524900 | −1.78211700 | −0.30890700 |
| H | −0.70688800 | −2.20615300 | 0.64103300 |
| H | −1.01480600 | −2.16802100 | −1.09549000 |
| F | 1.42927700 | 2.79903800 | −0.16126700 |
| H | −1.27552600 | 1.62894100 | −0.00695500 |
| N | −2.34687500 | 0.12478000 | 0.11474500 |
| C | −2.77268100 | −0.38105100 | 1.41444700 |
| H | −3.75140100 | 0.02912900 | 1.68799100 |
| H | −2.82990700 | −1.47157600 | 1.37792800 |
| H | −2.02964300 | −0.07311500 | 2.15154900 |
| C | −3.20662000 | −0.27556600 | −0.99287400 |
| H | −3.25266800 | −1.36634100 | −1.03924600 |
| H | −4.21700100 | 0.12969700 | −0.86620700 |
| H | −2.76937400 | 0.11616800 | −1.91213100 |
| O | −2.28322100 | 1.49144100 | 0.16412200 |

9

Lowest three frequencies (cm⁻¹): 18.78, 27.04, 38.19

E(RM062X)=−991.375222368

| | | | |
|---|---|---|---|
| C | −5.69733200 | −0.78406700 | 1.08409800 |
| C | −4.49468400 | −1.57956700 | 0.49576700 |
| C | −4.90559600 | 1.25176100 | −0.68990800 |
| C | −3.08468900 | −0.97571000 | 0.66702000 |
| C | −3.75135300 | 0.89924200 | −0.70423300 |
| C | −2.65813600 | −0.04137300 | −0.47976300 |
| H | −5.32793700 | −0.00240700 | 1.75949200 |
| H | −4.67113200 | −1.78095800 | −0.56835000 |
| H | −3.03321400 | −0.39610900 | 1.59560800 |
| H | −6.26973200 | −1.47314900 | 1.71251300 |
| H | −4.47688100 | −2.56057400 | 0.97935300 |
| H | −2.33689600 | −1.77207600 | 0.73007000 |
| H | −2.45311400 | −0.62634500 | −1.38224900 |
| C | −6.69320500 | −0.15646100 | 0.08371400 |
| H | −7.69278000 | −0.12580200 | 0.52990600 |
| H | −6.76450600 | −0.79655900 | −0.80266900 |
| C | −6.33006000 | 1.27840900 | −0.37078700 |
| H | −6.50965600 | 1.98750400 | 0.44513800 |
| O | −1.45648400 | 0.66859500 | −0.12703200 |
| C | −0.31126200 | −0.01446800 | −0.31068200 |
| O | −0.23782400 | −1.14473000 | −0.73518700 |
| N | 0.73863700 | 0.78883300 | 0.06172300 |
| H | 0.47560600 | 1.70972100 | 0.38282900 |
| C | 2.09962300 | 0.47965600 | 0.03985300 |
| C | 2.98476800 | 1.47608600 | 0.48070200 |
| C | 2.59995000 | −0.75667900 | −0.39452300 |
| C | 4.34940400 | 1.25241800 | 0.49287500 |
| H | 2.59305700 | 2.43196600 | 0.81553600 |
| C | 3.96933600 | −0.97923300 | −0.38116000 |
| H | 1.92005900 | −1.52329900 | −0.73570100 |
| C | 4.82453600 | 0.02090500 | 0.05969100 |
| H | 5.04783300 | 2.00812600 | 0.82919500 |
| H | 4.38446200 | −1.92356700 | −0.71065800 |
| N | 6.26742300 | −0.22515200 | 0.06949500 |
| O | 6.65732000 | −1.31206600 | −0.31685700 |
| O | 6.98895800 | 0.67374800 | 0.46426200 |
| H | −6.95114700 | 1.58788100 | −1.21601000 |

9-TS

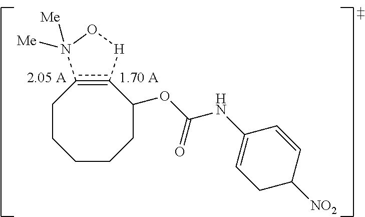

Lowest three frequencies (cm⁻¹): −136.00, 17.92, 22.89

E(RM062X)=−1201.67730053

| | | | |
|---|---|---|---|
| C | −4.38614700 | 2.68512900 | −0.67086600 |
| C | −2.88110800 | 2.98010600 | −0.49513100 |
| C | −4.09571100 | −0.12491600 | 0.27992600 |
| C | −1.86372600 | 1.90456800 | −0.89809500 |
| C | −2.85392700 | −0.12269900 | 0.17417500 |
| C | −1.69063300 | 0.76355400 | 0.10627800 |
| H | −4.53137800 | 2.04868700 | −1.55375500 |
| H | −2.68832400 | 3.28969400 | 0.54075400 |
| H | −2.12962700 | 1.47280000 | −1.86988900 |
| H | −4.86909900 | 3.63810800 | −0.90835000 |
| H | −2.64975400 | 3.85708500 | −1.10865800 |
| H | −0.88620600 | 2.38615700 | −1.00372100 |
| H | −1.46421700 | 1.17942000 | 1.09441900 |
| C | −5.13253100 | 2.07538200 | 0.53407600 |
| H | −6.10218100 | 2.56965900 | 0.65462900 |
| H | −4.56881100 | 2.26626000 | 1.45456200 |
| C | −5.38960300 | 0.56596900 | 0.41967200 |
| H | −6.02529900 | 0.36844200 | −0.45350400 |
| O | −0.53538500 | −0.03216900 | −0.29095400 |
| C | 0.65991600 | 0.45066300 | 0.07262600 |
| O | 0.84589000 | 1.48127600 | 0.68100800 |
| N | 1.63495200 | −0.42194700 | −0.35835600 |
| H | 1.28657500 | −1.24012000 | −0.83732900 |
| C | 3.01459300 | −0.30970000 | −0.19798900 |
| C | 3.80378600 | −1.34559900 | −0.72429700 |
| C | 3.62871900 | 0.77246800 | 0.45118000 |
| C | 5.18136100 | −1.31190400 | −0.61096800 |
| H | 3.32526600 | −2.18188600 | −1.22518500 |
| C | 5.01089500 | 0.80452700 | 0.56335000 |
| H | 3.02282800 | 1.56854200 | 0.85795300 |
| C | 5.76921400 | −0.23101600 | 0.03456900 |
| H | 5.80525900 | −2.10147300 | −1.01020400 |
| H | 5.51122800 | 1.62714100 | 1.05896000 |
| N | 7.22602500 | −0.18622400 | 0.15859800 |
| O | 7.71587600 | 0.77341500 | 0.72662900 |
| O | 7.86089200 | −1.11260300 | −0.31478100 |
| H | −5.93643400 | 0.21108500 | 1.30129300 |
| H | −2.79706000 | −1.81377500 | 0.01691000 |
| N | −4.61898000 | −2.10080200 | 0.13768600 |
| O | −3.38016400 | −2.66315800 | −0.01822100 |
| C | −5.41715200 | −2.36268800 | −1.05441900 |
| H | −5.51837400 | −3.44057600 | −1.22410100 |
| H | −6.40710800 | −1.91697500 | −0.93208200 |
| H | −4.90784700 | −1.90597200 | −1.90426600 |
| C | −5.21534100 | −2.59001600 | 1.37553900 |
| H | −6.20194700 | −2.13879000 | 1.50480600 |
| H | −5.31082500 | −3.68152000 | 1.35510500 |
| H | −4.56313600 | −2.29623800 | 2.19895800 |

Lowest three frequencies (cm⁻¹): 80.24, 118.25, 176.11

E(RM062X)=−510.431597287

| | | | |
|---|---|---|---|
| C | −1.67436000 | 1.18196600 | −0.40845200 |
| C | −0.32963800 | 1.58007400 | 0.26692400 |
| C | −0.76385600 | −1.45652300 | −0.06369900 |
| C | 0.97268800 | 1.16247200 | −0.44240900 |
| C | 0.40989900 | −1.17745400 | −0.07532500 |
| C | 1.50492800 | −0.20197000 | −0.01613700 |
| H | −1.48927800 | 0.85541900 | −1.43969700 |
| H | −0.29796000 | 1.20361600 | 1.29604200 |
| H | 0.83584900 | 1.13304100 | −1.52780100 |
| H | −2.28308900 | 2.08742900 | −0.48968400 |
| H | −0.30818000 | 2.67019500 | 0.35093800 |
| H | 1.77580700 | 1.87555500 | −0.22891000 |
| C | −2.53922700 | 0.12193600 | 0.30852200 |
| H | −3.59733800 | 0.30229800 | 0.09245900 |
| H | −2.41556400 | 0.22915700 | 1.39161500 |
| C | −2.21744000 | −1.34098700 | −0.08372900 |
| H | −2.58554300 | −1.55164200 | −1.09394800 |
| H | −2.70466700 | −2.04469400 | 0.59673400 |
| F | 2.56186500 | −0.54323100 | −0.79918400 |
| F | 1.98169000 | −0.12093100 | 1.25897000 |

10-TS

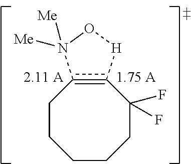

Lowest three frequencies (cm⁻¹): −118.45, 41.71, 43.87

E(RM062X)=−720.735442633

| | | | |
|---|---|---|---|
| C | 1.78908300 | 2.06449700 | −0.55671000 |
| C | 2.80860800 | 0.92348400 | −0.35069600 |
| C | −0.50780500 | 0.35230800 | 0.19602900 |
| C | 2.43944300 | −0.48862700 | −0.81765600 |
| C | 0.11225500 | −0.71562400 | 0.08518500 |
| C | 1.48323600 | −1.24875200 | 0.08740400 |
| H | 1.21974200 | 1.88579300 | −1.47867600 |
| H | 3.10090400 | 0.87636100 | 0.70444100 |
| H | 2.00889400 | −0.47065700 | −1.82418900 |
| H | 2.36682200 | 2.97628200 | −0.73679600 |
| H | 3.71590500 | 1.19352500 | −0.90078000 |
| H | 3.34797400 | −1.09898300 | −0.86117500 |
| C | 0.81552500 | 2.35434000 | 0.60613300 |
| H | 0.73506000 | 3.43529200 | 0.76082500 |
| H | 1.21375400 | 1.93061600 | 1.53481800 |
| C | −0.60349100 | 1.80550400 | 0.38972900 |
| H | −1.05521300 | 2.27659000 | −0.49268600 |
| H | −1.24088300 | 2.04720600 | 1.24793800 |
| F | 1.50294900 | −2.56302200 | −0.29587800 |
| F | 1.97960900 | −1.23328700 | 1.36473800 |
| H | −1.35352700 | −1.62930600 | −0.20414600 |
| N | −2.52977300 | −0.19823900 | −0.10633400 |
| C | −3.09078800 | 0.39299700 | −1.31369900 |
| H | −4.05539200 | −0.06475100 | −1.56362100 |
| H | −3.22560600 | 1.46665000 | −1.16027000 |
| H | −2.38702600 | 0.22259600 | −2.13003100 |
| C | −3.33511700 | 0.00951500 | 1.09001700 |
| H | −3.46900600 | 1.08199900 | 1.25228800 |
| H | −4.31576800 | −0.47100900 | 0.99103200 |
| H | −2.80052700 | −0.42733400 | 1.93478700 |
| O | −2.36330300 | −1.54678400 | −0.30544600 |

S18

Lowest three frequencies (cm⁻¹): 84.47, 121.18, 175.30

E(RM062X)=−426.4666974

| | | | |
|---|---|---|---|
| C | −1.73300600 | 1.15677900 | −0.40840600 |
| C | −0.39525900 | 1.58806600 | 0.26177300 |
| C | −0.77146900 | −1.46130700 | −0.02376100 |
| C | 0.91711500 | 1.18749600 | −0.44448100 |
| C | 0.38566000 | −1.11568600 | −0.05106700 |
| C | 1.52888700 | −0.18167200 | −0.04273400 |
| H | −1.53921300 | 0.81449200 | −1.43285300 |
| H | −0.37469900 | 1.22600200 | 1.29685900 |
| H | 0.74712800 | 1.16148600 | −1.52885700 |
| H | −2.35709700 | 2.05052600 | −0.50681200 |
| H | −0.39754400 | 2.68012500 | 0.33422100 |
| H | 1.69150100 | 1.94323700 | −0.26824300 |
| C | −2.58026300 | 0.09325400 | 0.32395600 |
| H | −3.64180700 | 0.25361900 | 0.10641900 |
| H | −2.45875700 | 0.22077400 | 1.40552800 |
| C | −2.22969500 | −1.36821100 | −0.04388700 |
| H | −2.59858000 | −1.59961000 | −1.04984000 |
| O | 2.56155000 | −0.53459400 | −0.94648900 |
| H | −2.71061100 | −2.06817000 | 0.64567400 |
| H | 2.15405200 | −0.70694600 | −1.80337500 |
| C | 2.19440900 | −0.12560300 | 1.32862000 |
| H | 2.57399000 | −1.11348200 | 1.59485200 |
| H | 3.03266300 | 0.57451000 | 1.28390600 |
| H | 1.48830100 | 0.20148200 | 2.09435500 |

S18-TS

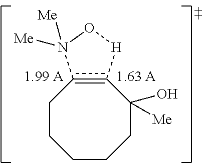

Lowest three frequencies (cm⁻¹): −187.48, 46.07, 52.59

E(RM062X)=−636.7643389

| | | | |
|---|---|---|---|
| C | −1.68999200 | 2.12745800 | 0.56967000 |
| C | −2.76121700 | 1.02803400 | 0.43710300 |
| C | 0.57426400 | 0.33503100 | −0.19723100 |
| C | −2.40082400 | −0.40642100 | 0.84145200 |
| C | −0.09183900 | −0.71852300 | −0.05941600 |
| C | −1.48388400 | −1.24394500 | −0.07345400 |
| H | −1.08494100 | 1.93637800 | 1.46608800 |
| H | −3.16652600 | 1.03940200 | −0.58272300 |
| H | −1.94869800 | −0.39256900 | 1.84298000 |
| H | −2.21837700 | 3.06736000 | 0.75927000 |
| H | −3.59875000 | 1.31587700 | 1.08282000 |
| H | −3.32944800 | −0.98282100 | 0.92764200 |
| C | −0.76060900 | 2.34614900 | −0.64197000 |
| H | −0.67171200 | 3.41719000 | −0.85285800 |
| H | −1.20070000 | 1.89040700 | −1.53654500 |
| C | 0.65516700 | 1.78760700 | −0.45027300 |
| H | 1.13354100 | 2.30102300 | 0.39472000 |
| O | −1.53729100 | −2.59462700 | 0.39416400 |
| H | 1.26309400 | 2.00192000 | −1.33781200 |
| H | −0.99826600 | −2.63590400 | 1.19205000 |
| H | 1.24989700 | −1.61226700 | 0.19765200 |
| N | 2.46289100 | −0.22552300 | 0.10719100 |
| C | 3.00775000 | 0.38523900 | 1.31591200 |
| H | 3.96444100 | −0.07785400 | 1.58021200 |
| H | 3.15075200 | 1.45568700 | 1.15187600 |
| H | 2.28880300 | 0.22519700 | 2.12049300 |
| C | 3.27620300 | −0.01588000 | −1.08692000 |
| H | 3.40465800 | 1.05565200 | −1.25646700 |
| H | 4.25628400 | −0.49082000 | −0.97042500 |
| H | 2.74939000 | −0.46587000 | −1.92912400 |
| O | 2.29614000 | −1.56352200 | 0.31329600 |
| C | −2.01316100 | −1.31791900 | −1.50474700 |
| H | −1.37752400 | −1.98744400 | −2.08708300 |
| H | −3.02967900 | −1.72302000 | −1.49520100 |
| H | −2.01841000 | −0.33464900 | −1.97834500 |

S19

Lowest three frequencies (cm⁻¹): 29.25, 209.73, 209.75

E(RM062X)=−155.9439042

| | | | |
|---|---|---|---|
| C | 0.00000000 | 0.00000000 | 0.60299000 |
| C | 0.00000000 | 0.00000000 | −0.60299000 |
| C | 0.00000000 | 0.00000000 | 2.06570800 |
| H | −0.51002300 | 0.88438900 | 2.45675800 |
| H | −0.51089200 | −0.88388800 | 2.45675800 |
| H | 1.02091500 | −0.00050100 | 2.45675800 |
| C | 0.00000000 | 0.00000000 | −2.06570800 |
| H | −1.02091500 | −0.00050100 | −2.45675800 |
| H | 0.51002300 | 0.88438900 | −2.45675800 |
| H | 0.51089200 | −0.88388800 | −2.45675800 |

S19-TS

Lowest three frequencies (cm⁻¹): −356.05, 62.60, 95.08

E(RM062X)=−366.217519

| | | | |
|---|---|---|---|
| C | 1.55364700 | −0.29356100 | −0.00038600 |
| C | 0.70913100 | 0.63801200 | −0.00196200 |
| C | 3.01745300 | −0.55067300 | 0.00020500 |
| N | −1.02763500 | −0.30894300 | 0.00011900 |
| C | −1.77383500 | −0.03174000 | 1.22452700 |
| H | −2.10624700 | 1.00897600 | 1.22606200 |
| H | −2.64230900 | −0.69483200 | 1.29783000 |
| C | −1.77405100 | −0.03744700 | −1.22548900 |
| H | −2.10383100 | 1.00408500 | −1.23355100 |
| H | −2.64433000 | −0.69869700 | −1.29412600 |
| O | −0.60849600 | −1.60041300 | 0.00312600 |
| H | 0.45876100 | −1.40079900 | 0.00222100 |
| H | −1.10531100 | −0.22433000 | −2.06630500 |
| H | −1.10413800 | −0.21152800 | 2.06617700 |
| H | 3.30073400 | −1.13740500 | 0.87915300 |
| H | 3.61902700 | 0.36485400 | −0.00495500 |
| H | 3.30037700 | −1.14716800 | −0.87224100 |
| C | 0.32711400 | 2.06639100 | −0.00094900 |
| H | 1.23858300 | 2.67128300 | −0.00490500 |
| H | −0.25716800 | 2.34274900 | −0.88438300 |
| H | −0.24949300 | 2.34282600 | 0.88750400 |

NMe₂OH

Lowest three frequencies (cm⁻¹): 251.20, 290.61, 315.61

E(RM062X)=−210.302452064

| | | | |
|---|---|---|---|
| N | 0.00000000 | 0.02490000 | −0.41697400 |
| C | −1.19858000 | −0.64238700 | 0.06738400 |
| H | −1.22931400 | −1.65128600 | −0.35177800 |
| H | −1.22122900 | −0.70365200 | 1.16601500 |
| C | 1.19858300 | −0.64238200 | 0.06738400 |
| H | 1.22932400 | −1.65127900 | −0.35178200 |
| H | 1.22123100 | −0.70365100 | 1.16601500 |
| O | −0.00000300 | 1.31390800 | 0.19839700 |
| H | −0.00000300 | 1.91418700 | −0.55581600 |
| H | 2.07548200 | −0.09062800 | −0.27480500 |
| H | −2.07548200 | −0.09063900 | −0.27480900 |

### Example 31: Bioorthogonal Reactions of Linear Alkynes

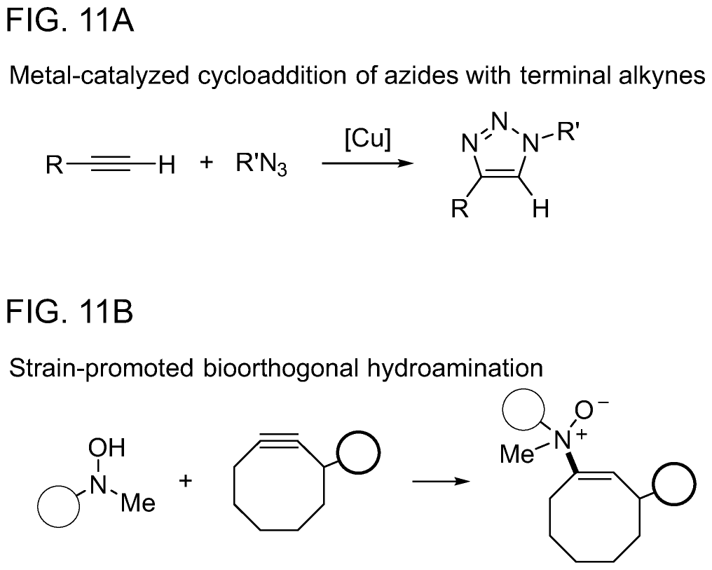

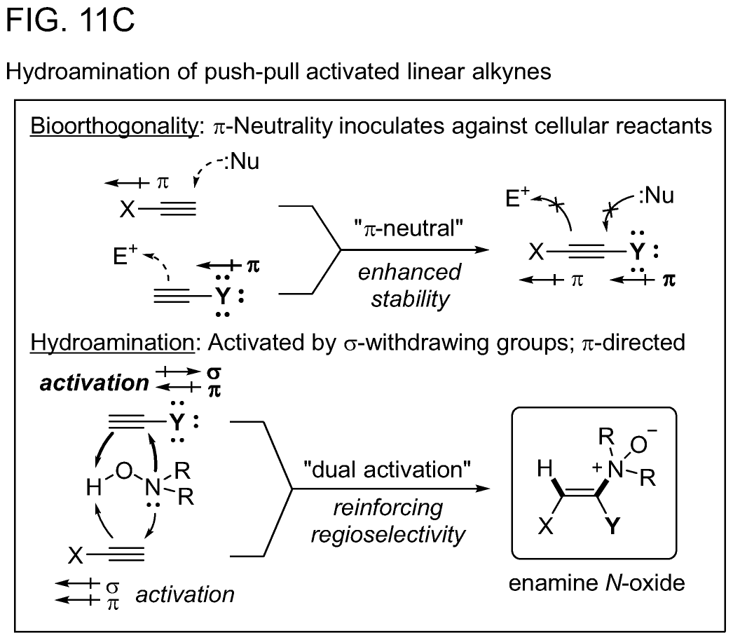

Modes of activation of the terminal alkyne are shown in FIG. 11. Polarized alkenes and alkynes lack chemoselectivity in biological contexts (Agard et al., J. Am. Chem. Soc. 126(46):15046-15047 (2004); McGrath et al., Chem. Sci. 3:3237-3240 (2012); Algar et al., Chemoselective and Bioorthogonal Ligation Reactions: Concepts and Applications, Wiley-VCH: Weinheim, 2017). A few enamine N-oxide products derived by intermolecular retro-Cope elimination have been reported, and of these few, each save one was the product of a reaction with a strong Michael acceptor (O'Neil et al., Chem. Commun. 50:7336-7339 (2014)).

The impact of inductively withdrawing halogen and chalcogen substituents at the propargylic and terminal positions of the alkyne were evaluated. Retro-Cope elimination reaction between propargyl ether 1′ and N,N-diethylhydroxylamine (2′) was completed in 18 hours whereas hydroamination of terminal alkyne 4′ was incomplete, even after 10 days. Terminal halogenation also had a similar accelerating effect as the conversion of 6-chlorohex-5-yn-1-ol (6′) was nearly complete by 24 hours. The regioselectivities induced by the propargyl and terminal alkyne substituents appeared to be reinforcing. Hydroamination of unactivated alkyne 4′ resulted in the preferential formation of the Markovnikov adduct, but this preference was overturned in favor of the anti-Markovnikov products for both propargyl ether 1′ and chloroalkyne 6′. These data intimated synergy should the propargylic and terminal substituent effects be combined (Scheme 1).

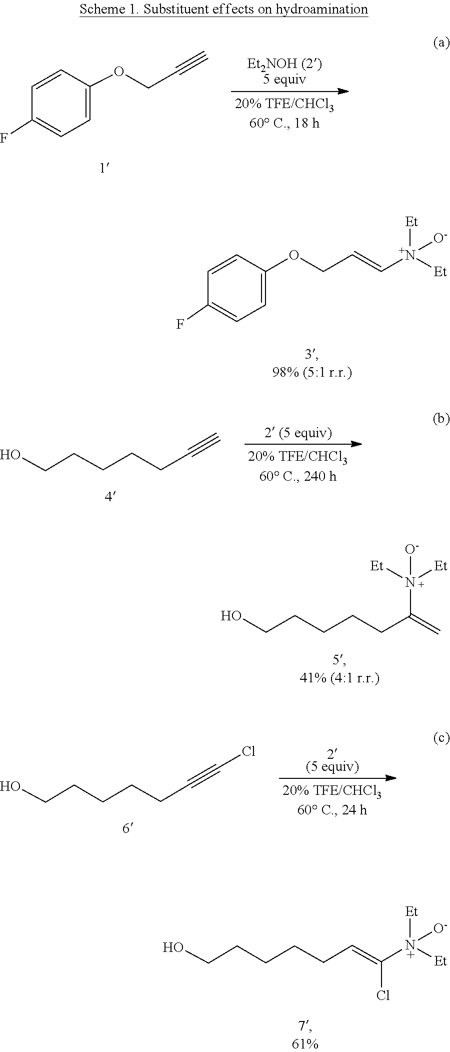

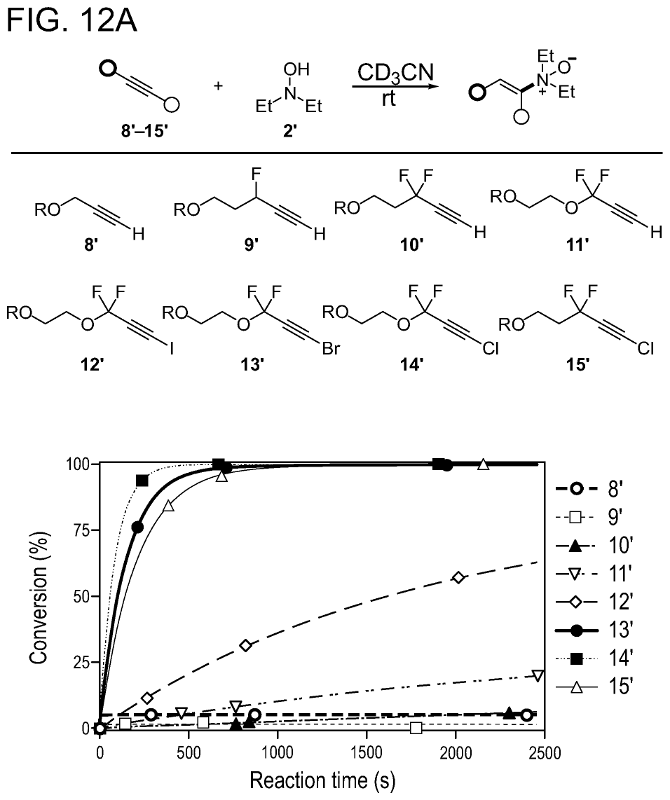

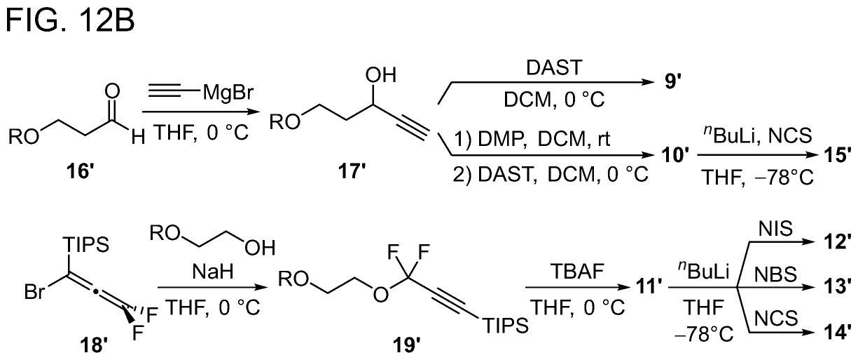

Substrates 8′-15′ were synthesized to examine regioselectivity (FIG. 12A). Grignard addition of ethynylmagnesium bromide into aldehyde 16′ provided propargyl alcohol 17′, which was readily converted to either propargyl fluoride 9′ using diethylaminosulfur trifluoride (DAST) or to propargyl difluoride 10′ by sequential oxidation and difluorination with Dess-Martin periodinane and DAST, respectively. Chloroalkyne 15′ was obtained by halogenation of the corresponding acetylide (FIG. 12B). Difluoropropargyl ethers 11′-14′ were accessed by S_{N}2′ addition of a sodium alkoxide into bromodifluoroallene 18′ (Xu et al., Angew. Chem., Int. Ed. 44(1):7404-7407 (2005)), desilylation, and acetylide halogenation (FIG. 12B).

With the desired alkynes in hand, their reactivity toward N,N-diethylhydroxylamine (2′) was examined. Alkynes 4′-11′ were each incubated with 5 equivalents of hydroxylamine 12′ in CD₃CN at room temperature, and the reaction conversions were monitored by ¹H (alkyne 8′) or ¹⁹F (alkynes 9′-15′) NMR (FIG. 12A). Robust reactivity toward N,N-dialkylhydroxylamines was observed with halogenated alkynes 13′-15′ while alkynes 11′ and 12′ exhibited moderate reactivity. Difluoroalkyne 10′ underwent partial conversion over 48 hours; however, no conversion was observed for propargyl fluoride 9′ and propargyl ether 8′ over the same period. Their conversion to the corresponding enamine N-oxides could only be achieved upon heating to 60° C. (Example 59). The data indicated that the rate of hydroamination correlated positively with the addition of electronegative substituents to the propargylic position as well as to alkyne termini.

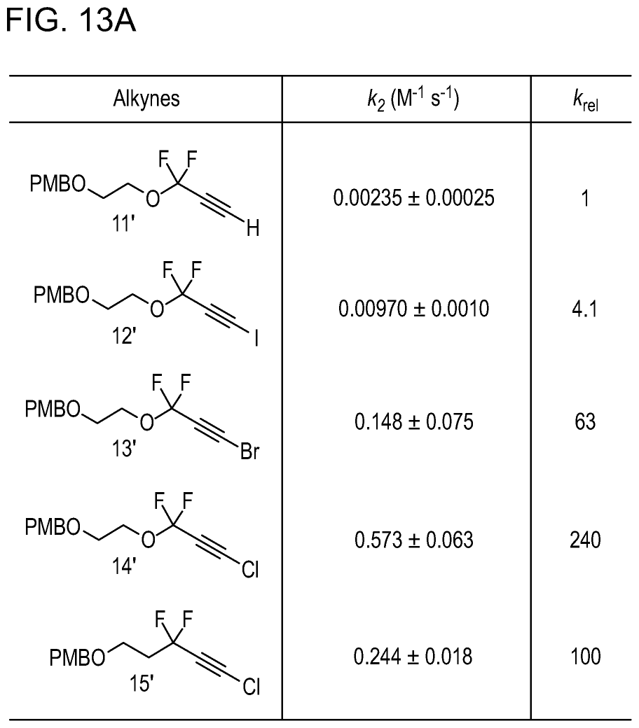

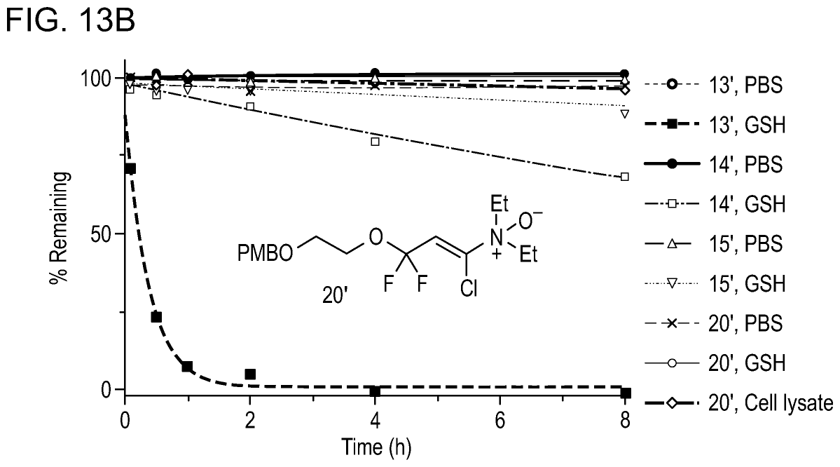

Second-order rate constants were determined for moderately reactive substrates 11′ and 12′ under pseudo first-order conditions using excess hydroxylamine. Rate constants for the most reactive substrates 13′-15′ were determined by reaction with equimolar hydroxylamine (FIG. 13A). Rate accelerations of 4.1, 63, and 240-fold were achieved over the parent difluoroether 11′ by addition of an iodine, bromine, or chlorine atom, respectively, to the alkyne terminus. Removal of a propargylic oxygen from chloroalkyne 14′ produced chloroalkyne 15′, which was still marginally faster than bromoalkyne 13′ which possesses the propargylic ether. With absolute rate constants on the order of 0.1-1 M-s⁻¹, the rate of hydroamination of alkynes 13′-15′ was comparable to the fastest bioorthogonal strain-promoted azide-alkyne cycloaddition reactions yet reported.

The stability of alkynes 13′-15′ under biological conditions was evaluated (FIG. 13B). Alkynes 14′ and 15′ showed no degradation by ¹⁹F NMR over 7 days in 50% CD₃CN/phosphate-buffered saline (PBS), pH 7.0 at room temperature while they displayed half-lives of 30 hours and 82 hours, respectively, in the added presence of 2 mM glutathione. These stabilities compared favorably with contemporary bioorthogonal transformations (Oliveira et al., Chem. Soc. Rev. 46(16):4895-4950 (2017); Tian et al., ACS Chem. Biol. 14(12):2489-2496 (2019)). Notably, bromoalkyne 13′, which was less reactive toward hydroamination than alkynes 14′ and 15′, proved unacceptably sensitive to thiols, degrading in <10 minutes under identical conditions. This observation was consistent with σ-withdrawing/n-donating alkyne substituents, such as halogens, simultaneously promoting hydroamination and attenuating conjugate addition by cellular nucleophiles.

The enamine N-oxide products were remarkably stable, especially in aqueous solutions. Enamine N-oxide 20′ showed no observable degradation over 24 hours in cell lysate at room temperature.

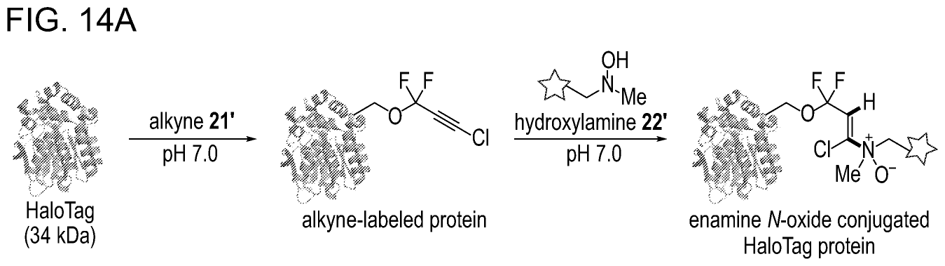

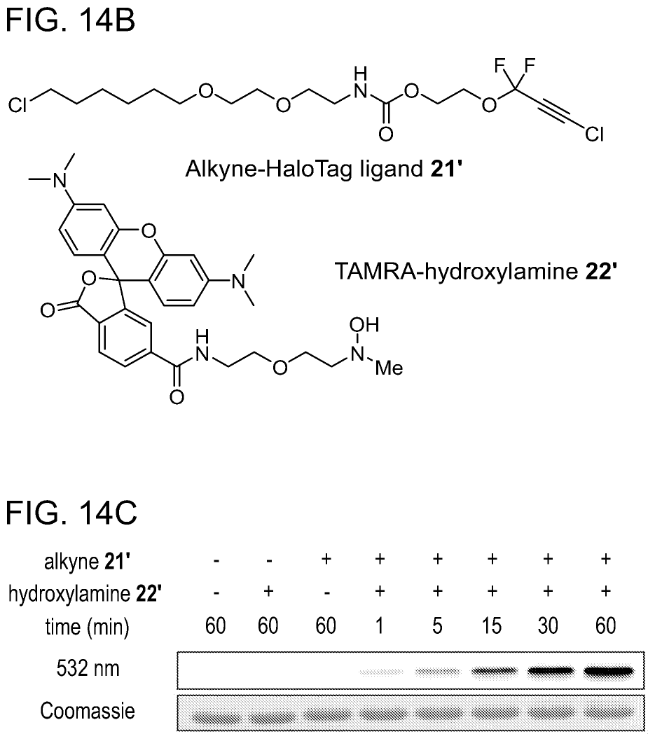

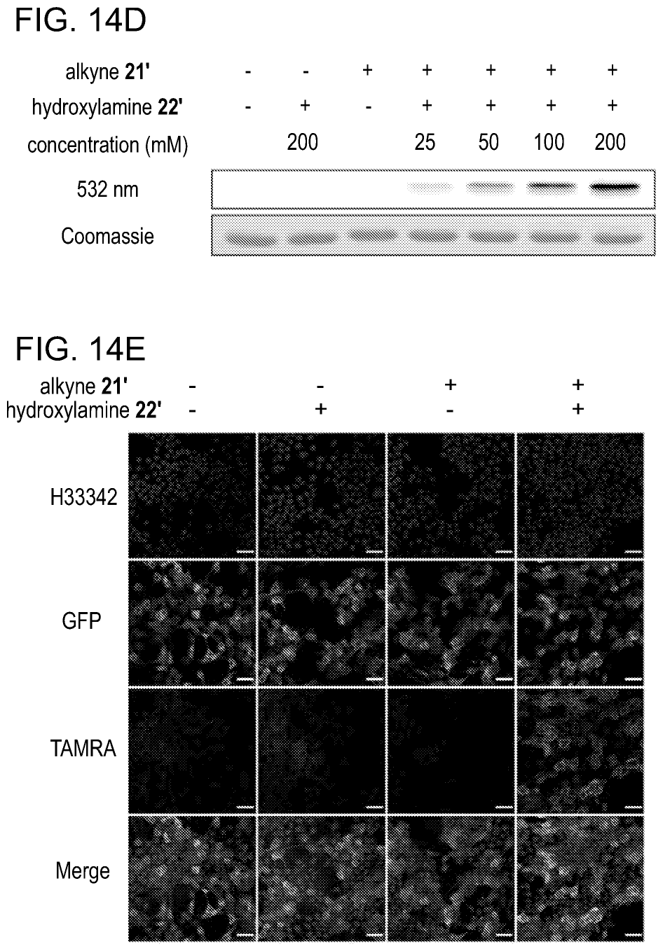

The viability of the reaction was evaluated both in vitro and in cells (FIG. 14A-FIG. 14B). Purified recombinant HaloTag protein (Los et al., ACS Chem. Biol. 3(6):373-382 (2008); Murrey et al., J. Am. Chem. Soc. 137(35):11461-11475 (2015)) was incubated with HaloTag linker-conjugated difluoropropargyl ether 21′ for 10 minutes at room temperature in pH 7.0 phosphate buffer to provide alkyne-conjugated protein. This protein was then treated with 200 μM TAMRA-N-methylhydroxylamine 22′ and analyzed at various time points by in-gel fluorescence. Fluorophore-labeled protein was observed within 1 minute, and the experiment demonstrated time-dependent labeling over 1 hour (FIG. 14C). Labeling also proved concentration-dependent across a range of concentrations up to 200 μM (FIG. 14D). No labeling was observed in the absences of either the alkyne or hydroxylamine.

Live cell labeling by hydroamination was explored. HEK293T cells were transiently transfected with a cell surface HaloTag-GFP construct, treated with 10 μM HaloTag linker-conjugated difluoropropargyl ether 21′, washed, and incubated with 10 μM TAMRA-conjugated hydroxylamine 22′ for 1 hour. The cells were then fixed and visualized by confocal microscopy. TAMRA signal from cells treated with alkyne 21′ and hydroxylamine 22′ localized at the cell surface and co-localized with the GFP signal of transfected cells. Importantly, negative controls lacking the alkyne and/or hydroxylamine did not exhibit labeling. These experiments demonstrated the specificity and efficacy of the reaction in cellular ligation applications.

The role of rehybridization energy in driving the hydroamination of electronically modified linear alkynes was examined. Underwhelming improvement in reactivity was noted from bromoalkyne 13′ to chloroalkyne 14′. Deviation of the atomic orbitals from canonical hybridization schemes in accordance with Bent's rule was expected to result in significant ground state destabilization of haloalkynes—instability (Hanamoto et al., Angew. Chem., Int. Ed. 43(27):3582-3584 (2004); Alabugin et al., J. Comput. Chem. 28(1):373-390 (2007)), which the reaction was expected to alleviate.

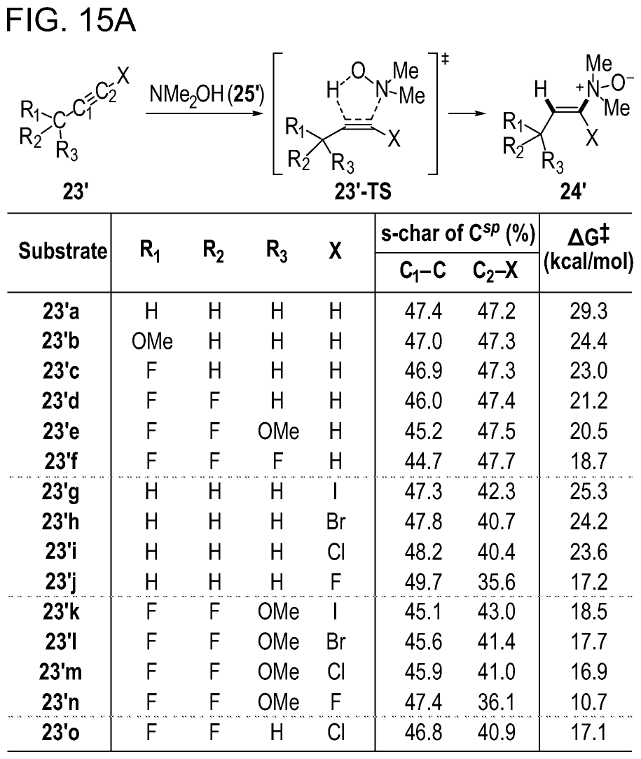

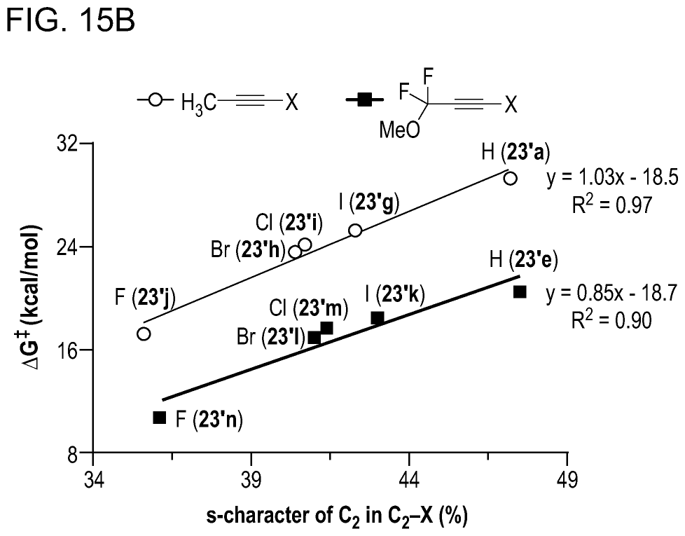

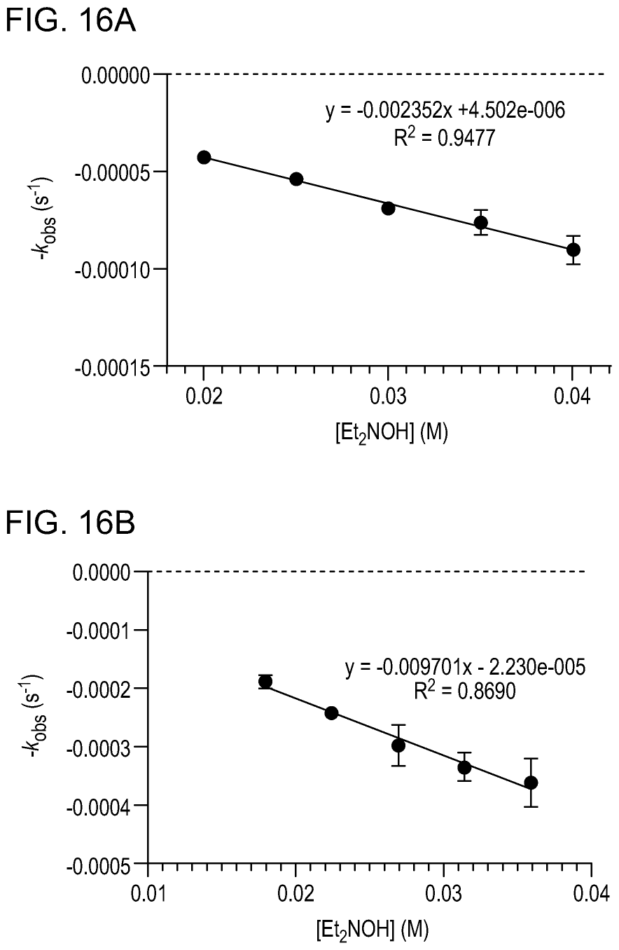

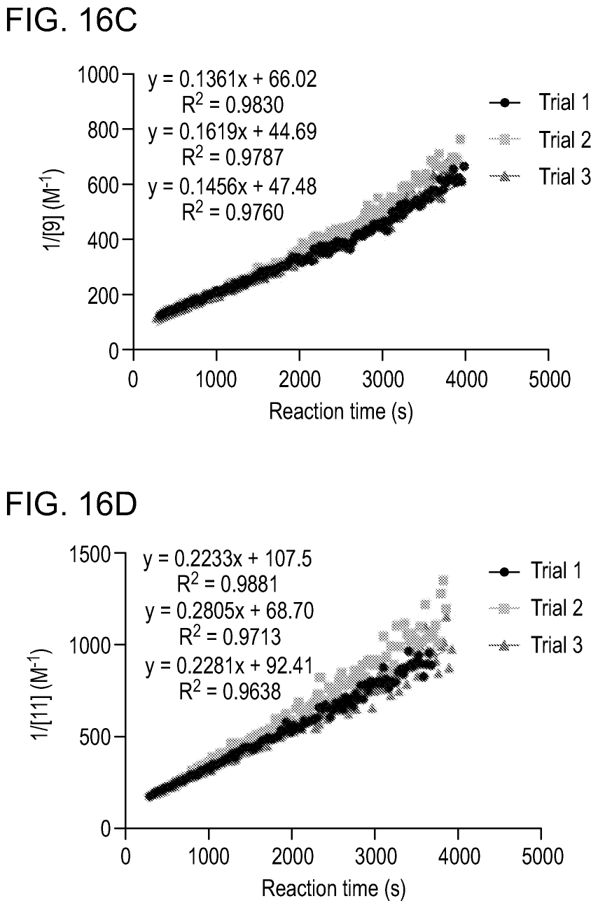

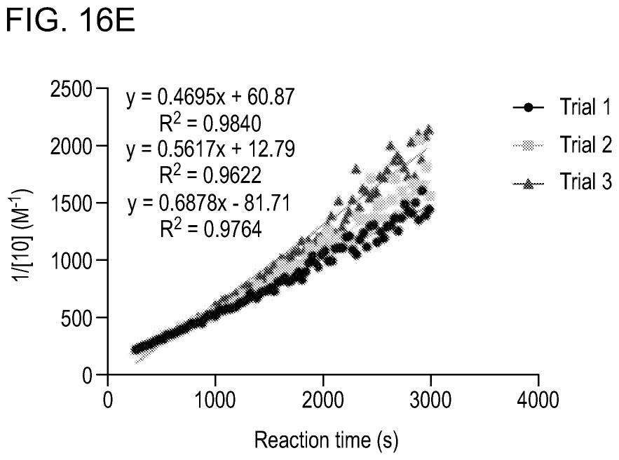

DFT calculations performed at the M06-2X (Zhao et al., Theor. Chem. Acc. 120:215-241 (2008)) level of theory for reactions between N,N-dimethylhydroxylamine (25′) and model alkynes 23′a-23′o produced activation energies (ΔG^{‡}) that accurately recapitulated the reactivity trends observed experimentally (FIG. 15A). To obtain insight pertinent to the influence of rehybridization energy, natural bond orbital analysis was performed. In ground state calculations of the alkyne component, electronegative atoms, whether at the propargylic or terminal position, reduced the s-character in the bonding orbital of the most proximal sp-hybridized carbon consistent with Bent's rule. Notably, the effect of terminal halogenation was much more pronounced, and the s-character of C1 in the C1-X bond deviated significantly (Cl, 40%; F, 35%) from the canonical 50%.

A plot of the reaction free energies against the percent s-character of C1 in the C1-X bond of terminally functionalized propynes or of difluoropropargyl ethers revealed a strong positive linear relationship consistent with other reactions that exhibit rate enhancements deriving from substantial rehybridization effects (FIG. 15B). NBO analysis also revealed that propargylic modifications had a much more muted effect on alkyne rehybridization despite its more significant impact on reducing the activation free energy. In combination with the divergent regioselectivities imparted by each modification, it is plausible that the accelerating effects of propargylic halogen substituents can be imputed more heavily on their stereoelectronic rather than inductive effects, while the opposite may be true for their terminal counterparts. Nonetheless, alkyne halogenation at either site or in combination provides an effective alternative to strain-activation in the context of the retro-Cope elimination reaction.

A bioorthogonal reaction between N,N-dialkylhydroxylamines and halogenated alkynes was described. The electronic effects, when competing mesomeric and inductive factors are properly balanced, sufficiently activate a linear alkyne in the uncatalyzed conjugative retro-Cope elimination reaction while adequately inoculating it against cellular nucleophiles. This design preserved the low-profile of an alkyne and paired it with a comparably unobtrusive hydroxylamine. The kinetics were on par with those of the fastest strain-promoted azide-alkyne cycloaddition reactions, the products regioselectively formed, the components sufficiently stable and easily installed, and the reaction was suitable for cellular labeling.

### Example 32: Synthesis of 7-chlorohept-6-yn-1-ol (6′)

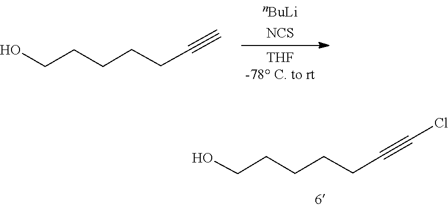

n-Butyllithium (3.34 mL, 8.36 mmol, 2.5 M in hexanes) was added dropwise to a solution of hept-6-yn-1-ol (500 μL, 3.98 mmol) in THE (40 mL) at −78° C. The reaction mixture stirred at −78° C. for 30 minutes. N-chlorosuccinimide (796 mg, 5.96 mmol) was then added to the reaction mixture. The ice bath was immediately removed, and the solution was allowed to warm to room temperature. After 2 hours, the reaction mixture was diluted with diethyl ether (100 mL) and washed with water (100 mL). The resulting organic layer was dried over anhydrous magnesium sulfate, filtered, and concentrated under reduced pressure. The crude mixture was purified by flash column chromatography on silica gel (eluent: 30% ethyl acetate in hexanes) to provide chloroalkyne 6′ (156 mg, 27%) as a yellow oil. ¹H NMR (500 MHz, CDCl₃) δ 3.63 (t, J=6.6 Hz, 2H), 2.17 (t, J=6.9 Hz, 2H), 1.60-1.48 (m, 4H), 1.47-1.40 (m, 2H). ¹³C NMR (126 MHz, CDCl₃) δ 69.6, 63.0, 57.4, 32.4, 28.3, 25.2, 18.9. FTIR (thin film) cm⁻¹: 3306 (br), 2937 (s), 2863 (m), 1055 (s), 999 (m). HRMS (ESI) (m/z): calc'd for C₇H₁₂ClO [M+H]⁺: 147.0571, found: 147.0573.

### Example 33: Synthesis of (Z)-1-chloro-N,N-diethyl-7-hydroxyhept-1-en-1-amine oxide (7′)

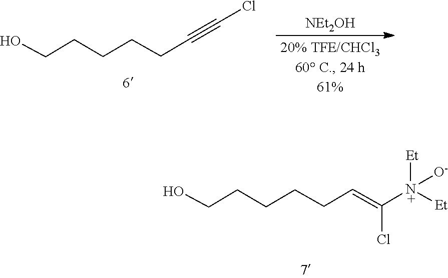 [SMILES: OCCCCCC#CCl ; CC[N+]([O-])(CC)/C(Cl)=C/CCCCCO ; "NEt2OH"]

N,N-Diethylhydroxylamine (175 μL, 1.71 mmol) was added to a solution of chloroalkyne 6′ (50.0 mg, 341 μmol) in 20% 2,2,2-trifluoroethanol/chloroform (v/v, 1.54 mL). The reaction mixture stirred at 60° C. for 24 hours. The reaction mixture was diluted with chloroform and purified by flash column chromatography on silica gel (eluent: 30% CMA in chloroform). To provide enamine N-oxide 7′ (48.8 mg, 61%) as a yellow oil. ¹H NMR (500 MHz, CD₃OD) δ 7.00 (t, J=7.6 Hz, 1H), 3.84-3.72 (m, 2H), 3.55 (t, J=6.6 Hz, 2H), 3.38-3.31 (m, 2H), 2.31 (q, J=7.4 Hz, 2H), 1.64-1.48 (m, 4H), 1.45-1.39 (m, 2H), 1.21 (t, J=7.1 Hz, 6H). ¹³C NMR (126 MHz, CD₃OD) δ 134.7, 130.2, 64.6, 62.9, 33.4, 29.1, 28.8, 26.7, 8.3. FTIR (thin film) cm⁻¹: 3273 (br), 2933 (s), 2859 (m), 1655 (w), 1454 (m), 1375 (m). HRMS (ESI) (m/z): calc'd for C₁₁H₂₃C₁NO₂ [M+H]⁺: 236.1412, found: 236.1412.

### Example 34: Synthesis of (E)-N,N-diethyl-3-((4-methoxybenzyl)oxy)prop-1-en-1-amine oxide (S1′)

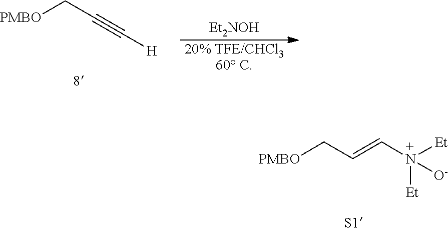

N,N-diethylhydroxylamine (165 μL, 1.60 mmol) was added via syringe to a solution of 1-methoxy-4-((prop-2-yn-1-yloxy)methyl)benzene (8′, 51.2 mg, 320 μmol) (Kramer et al., Adv. Synth. Catal. 350:1131-1148 (2008)) in 20% 2,2,2-trifluoroethanol/chloroform (v/v, 1.5 mL). The reaction mixture was then heated to 60° C. and stirred for 17 hours. The reaction mixture was concentrated under reduced pressure. The crude mixture was purified by flash column chromatography on silica gel (eluent: 30% CMA in chloroform) to provide enamine N-oxide S1′ (65.3 mg, 87%) as a colorless oil. ¹H NMR (500 MHz, CD₃OD) δ 7.27 (d, J=8.2 Hz, 2H), 6.90 (d, J=8.4 Hz, 2H), 6.47 (dt, J=13.1, 5.0 Hz, 1H), 6.22 (dt, J=13.2, 1.8 Hz, 1H), 4.48 (s, 2H), 4.14 (dd, J=5.0, 1.8 Hz, 2H), 3.78 (s, 3H), 3.37-3.33 (m, 4H), 1.25 (t, J=7.1 Hz, 6H). ¹³C NMR (126 MHz, CD₃OD) δ 161.1, 139.3, 131.4, 130.7, 127.7, 115.0, 73.6, 67.4, 65.2, 55.8, 8.7. FTIR (thin film) cm⁻¹: 3228 (br), 2982 (w), 2941 (w), 1655 (w), 1610 (m), 1513 (s), 1245 (s), 1029 (s), 816 (s). HRMS (ESI) (m/z): calc'd for C₁₅H₂₄NO₃ [M+H]⁺: 266.1751, found: 266.1748.

### Example 35: Synthesis of 5-((4-methoxybenzyl)oxy)pent-1-yn-3-ol (17′)

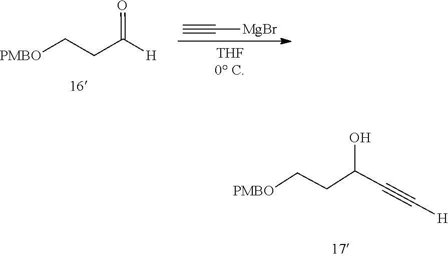

Ethynylmagnesium bromide (0.5 M in THF, 24.8 mL, 12.4 mmol) was added dropwise via syringe to a solution of 3-((4-methoxybenzyl)oxy)propanal (S2′, 2.00 g, 10.3 mmol) (Arikan et al., Org. Lett. 10(16):3521-3524 (2008)) in THF (50 mL) at 0° C. After 2 hours, the reaction mixture was diluted with ethyl acetate (100 mL), and washed sequentially with saturated aqueous ammonium chloride solution (100 mL) and brine (100 mL). The resulting organic layer was dried over anhydrous magnesium sulfate, filtered, and concentrated under reduced pressure. The crude mixture was purified by flash column chromatography on silica gel (eluent: 30% ethyl acetate in hexanes) to afford the desired alcohol 17′ (1.74 g, 77%) as a light yellow oil. Rf: 0.48 (30% ethyl acetate in hexanes). ¹H NMR (500 MHz, CDCl₃) δ 7.23 (d, J=8.7 Hz, 2H), 6.86 (d, J=8.6 Hz, 2H), 4.58 (ddd, J=6.6, 4.4, 2.1 Hz, 1H), 4.49-4.39 (m, 2H), 3.86-3.75 (m, 4H), 3.69-3.58 (m, 1H), 2.43 (d, J=2.1 Hz, 1H), 2.11-2.00 (m, 1H), 1.96-1.84 (m, 1H). ¹³C NMR (126 MHz, CDCl₃) δ 159.5, 130.0, 129.6, 114.0, 84.5, 73.3, 73.1, 67.5, 61.6, 55.5, 36.7. FTIR (thin film) cm⁻¹: 3399 (br), 3283 (w), 2930 (w), 2863 (w), 1610 (m), 1513 (s), 1245 (s). HRMS (ESI) (m/z): calc'd for C₁₃H₁₆NaO₃ [M+Na]⁺: 243.0992, found: 243.0989.

### Example 36: Synthesis of 1-(((3-fluoropent-4-yn-1-yl)oxy)methyl)-4-methoxybenzene (9′)

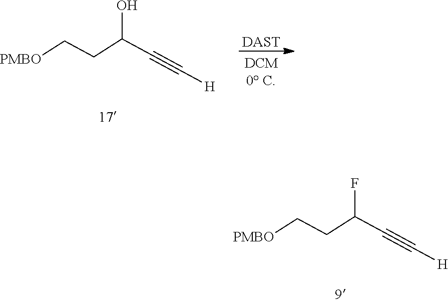

Diethylaminosulfur trifluoride (DAST, 504 μL, 3.81 mmol) was added dropwise via syringe to a solution of 17′ (800 mg, 3.63 mmol) in DCM (25 mL) at 0° C. After 80 minutes, the reaction mixture was concentrated under reduced pressure. The crude mixture was purified by flash column chromatography on silica gel (eluent: hexane and 5% ethyl acetate in hexanes) to provide fluoroalkyne 9′ (379 mg, 47%) as a light yellow oil. Rf: 0.29 (5% ethyl acetate in hexanes). ¹H NMR (500 MHz, CDCl₃) δ 7.24 (d, J=7.9 Hz, 2H), 6.87 (d, J=7.8 Hz, 3H), 5.41-5.15 (m, 1H), 4.43 (s, 2H), 3.79 (s, 3H), 3.66-3.55 (m, 2H), 2.66 (dt, J=5.6, 1.9 Hz, 1H), 2.34-1.96 (m, 2H). ¹³C NMR (126 MHz, CDCl₃) δ 159.5, 130.4, 129.5, 114.0, 80.3 (d, J=25.5 Hz), 80.1 (d, J=166.9 Hz), 76.7 (d, J=10.7 Hz), 73.0, 65.1 (d, J=4.9 Hz), 55.5, 36.5 (d, J=22.7 Hz). ¹⁹F NMR (471 MHz, CDCl₃) δ −178.6. FTIR (thin film) cm⁻¹: 3295 (w), 2937 (w), 2866 (w), 1610 (m), 1588 (w), 1513 (S), 1245 (s), 1174 (m), 1088 (s), 1033 (s). HRMS (GC-MS) (m/z): calc'd for C₁₃H₁₅FO₂ [M]⁺: 222.1051, found: 222.1049.

### Example 37: Synthesis of (E)-N,N-diethyl-3-fluoro-5-((4-methoxybenzyl)oxy)pent-1-en-1-amine oxide (S2′)

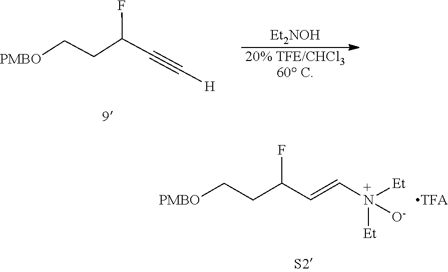

N,N-Diethylhydroxylamine (103 μL, 1.00 mmol) was added to a solution of fluoroalkyne 9′ (44.5 mg, 200 μmol) in 20% 2,2,2-trifluoroethanol/chloroform (v/v, 1 mL). The reaction mixture was then heated to 60° C. and stirred for 12 hours. Upon completion of the reaction as determined by TLC, the reaction mixture was diluted with chloroform and purified by flash column chromatography on silica gel (eluent: 20% CMA in chloroform). The material was re-purified by preparatory high-performance liquid chromatography (HPLC) using a C₁s reverse phase column (250×21.2 mm, 5 μm particle size, 20 mL/min flow rate, eluent: 40% MeCN/H₂O+0.1% TFA (2 min), gradient 40→100% MeCN/H₂O+0.1% TFA (20 min), t_{R}=12.83 min) to provide enamine N-oxide S2′ (27.5 mg, 44%) as a colorless oil. Rf: 0.22 (40% CMA in chloroform). ¹H NMR (500 MHz, CD₃OD) δ 7.27 (d, J=8.6 Hz, 2H), 6.90 (d, J=8.7 Hz, 2H), 6.59 (ddd, J=18.5, 13.4, 4.7 Hz, 1H), 6.40 (d, J=13.4 Hz, 1H), 5.51-5.24 (m, 1H), 4.44 (s, 2H), 3.83-3.71 (m, 7H), 3.67-3.56 (m, 2H), 2.16-1.97 (m, 2H), 1.33 (td, J=7.1, 1.3 Hz, 6H). ¹³C NMR (126 MHz, CD₃OD) δ 161.1, 133.8 (d, J=14.7 Hz), 132.9 (d, J=18.1 Hz), 131.6, 130.8, 114.9, 89.0 (d, J=172.8 Hz), 74.0, 66.1 (d, J=5.5 Hz), 65.3 (d, J=3.8 Hz), 55.8, 36.5 (d, J=21.2 Hz), 8.3. ¹⁹F NMR (471 MHz, CD₃OD) δ −77.3, −185.4. FTIR (thin film) cm⁻¹: 3220 (br), 2940 (w), 2870 (w), 1610 (m), 1513 (s), 1461 (m), 1245 (s), 1092 (s), 1033 (s). HRMS (ESI) (m/z): calc'd for C₁₇H₂₇FNO₃ [M+H]⁺: 312.1969, found: 312.1967.

### Example 38: Synthesis of 5-((4-methoxybenzyl)oxy)pent-1-yn-3-one (S3′)

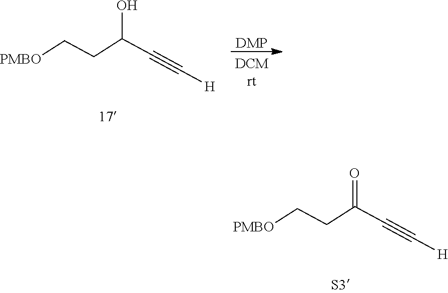

Dess-Martin periodinane (DMP, 4.64 g, 10.9 mmol) was added to a solution of alcohol 17′ (2.19 g, 9.94 mmol) in DCM (100 mL) at room temperature. After 80 minutes, the reaction was quenched with 50% saturated aqueous sodium thiosulfate solution/saturated sodium aqueous bicarbonate solution (v/v, 100 mL). The resulting organic layer was separated, dried over anhydrous sodium sulfate, filtered, and concentrated under reduced pressure. The crude mixture was purified by flash column chromatography on silica gel (eluent: 20% ethyl acetate in hexanes) to provide ynone S3′ (1.42 g, 65%) as a clear yellow oil. Rf: 0.50 (20% ethyl acetate in hexanes). ¹H NMR (500 MHz, CDCl₃) δ 7.23 (d, J=8.6 Hz, 2H), 6.85 (d, J=8.6 Hz, 2H), 4.44 (s, 2H), 3.93-3.50 (m, 5H), 3.21 (s, 1H), 2.83 (t, J=6.1 Hz, 2H). ¹³C NMR (126 MHz, CDCl₃) δ 185.3, 159.5, 130.1, 129.6, 114.0, 81.4, 79.1, 73.1, 64.4, 55.5, 45.7. FTIR (thin film) cm⁻¹: 3358 (br), 3257 (w), 2907 (w), 2866 (w), 2091 (m), 1681 (s), 1610 (m), 1513 (s), 1245 (s), 1174 (m), 1092 (s), 1033 (s), 813 (m). HRMS (ESI) (m/z): calc'd for C₁₃H₁₄KO₃ [M+K]⁺: 257.0575, found: 257.0572.

### Example 39: Synthesis of 1-(((3,3-difluoropent-4-yn-1-yl)oxy)methyl)-4-methoxybenzene (10′)

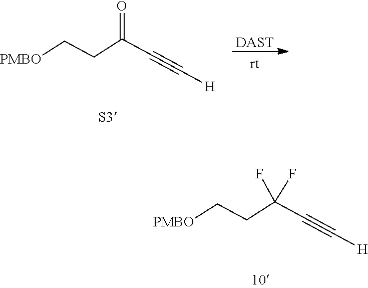

Diethylaminosulfur trifluoride (DAST, 2.18 mL, 16.5 mmol) was added via syringe to a vial containing neat ynone S3′ (1.20 g, 5.50 mmol) at room temperature. After 23 hours, the reaction mixture was diluted with DCM (60 mL) and washed with saturated aqueous sodium bicarbonate solution (60 mL). The aqueous layer was extracted with DCM (3×60 mL), dried over anhydrous sodium sulfate, filtered, and concentrated under reduced pressure. The residue was purified by flash column chromatography on silica gel (eluent: 7.5% ethyl acetate in hexanes) to provide difluoroalkyne 10′ (765 mg, 58%) as a yellowish oil. Rf: 0.53 (10% ethyl acetate in hexanes). ¹H NMR (500 MHz, CDCl₃) δ 7.25 (d, J=8.7 Hz, 2H), 6.87 (d, J=8.6 Hz, 2H), 4.45 (s, 2H), 3.78 (s, 3H), 3.68 (t, J=6.9 Hz, 2H), 2.76 (t, J=5.0 Hz, 1H), 2.39 (tt, J=14.8, 6.9 Hz, 2H). ¹³C NMR (126 MHz, CDCl₃) δ 159.5, 130.1, 129.5, 114.0, 113.1 (t, J=233.3 Hz), 76.4 (t, J=40.4 Hz), 75.8 (t, J=6.9 Hz), 73.0, 63.9 (t, J=4.8 Hz), 55.4, 39.5 (t, J=25.6 Hz). ¹⁹F NMR (471 MHz, CDCl₃) δ −82.7. FTIR (thin film) cm⁻¹: 3299 (w), 2937 (w), 2874 (w), 2132 (m), 1614 (m), 1513 (s), 1245 (s), 1096 (s), 1033 (s). HRMS (ESI) (m/z): calc'd for C₁₃H₁₅F₂O₂ [M+H]⁺: 263.0854, found: 263.0853.

### Example 40: Synthesis of (E)-N,N-diethyl-3,3-difluoro-5-((4-methoxybenzyl)oxy)pent-1-en-1-amine oxide (S4′)

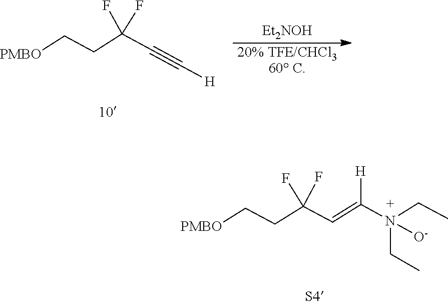

N,N-Diethylhydroxylamine (41.1 μL, 400 μmol) was added to a solution of difluoroalkyne 10′ (48.0 mg, 200 μmol) in 20% 2,2,2-trifluoroethanol/chloroform (v/v, 1 mL). The reaction mixture stirred at 60° C. for 7 hours. Upon completion of the reaction as determined by TLC, the reaction mixture was diluted with chloroform and purified by flash column chromatography on silica gel (eluent: 20% CMA in chloroform) to provide enamine N-oxide S4′ (43.1 mg, 65%) as a colorless oil. Rf: 0.08 (30% CMA in chloroform). ¹H NMR (500 MHz, CD₃OD) δ 7.27 (d, J=8.6 Hz, 2H), 6.90 (d, J=8.6 Hz, 2H), 6.68-6.56 (m, 2H), 4.43 (s, 2H), 3.78 (s, 3H), 3.62 (t, J=6.4 Hz, 2H), 3.48-3.28 (m, 4H), 2.55-2.20 (m, 2H), 1.21 (t, J=7.1 Hz, 6H). ¹³C NMR (126 MHz, CD₃OD) δ 161.0, 143.5, 131.5, 130.8, 126.6 (t, J=26.9 Hz), 121.8 (t, J=239.0 Hz), 114.9, 73.9, 65.3, 64.7 (t, J=5.9 Hz), 55.8, 39.0 (t, J=26.0 Hz), 8.6. ¹⁹F NMR (471 MHz, CD₃OD) δ −94.5. FTIR (thin film) cm⁻¹: 3358 (br), 3075 (w) 2941 (m), 2874 (w), 1689 (w), 1610 (m), 1513 (s), 1245 (s). HRMS (ESI) (m/z): calc'd for C₁₇H₂₅F₂NNaO₃ [M+Na]⁺: 352.1695, found: 352.1689.

### Example 41: Synthesis of 1-(((5-chloro-3,3-difluoropent-4-yn-1-yl)oxy)methyl)-4-methoxybenzene (15′)

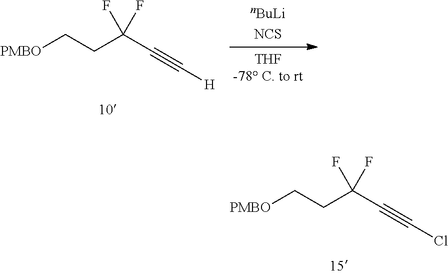

n-Butyllithium (638 μL, 1.50 mmol, 2.5 M in hexanes) was added dropwise via syringe to a solution of difluoroalkyne 10′ (300 mg, 1.25 mmol) in THE (10 mL) at −78° C. After 1 hour, N-chlorosuccinimide (250 mg, 1.88 mmol) was added. The ice bath was immediately removed, and the solution was allowed to warm to room temperature. After 1 hour, the reaction was quenched by the addition of water (1 mL) and saturated aqueous ammonium chloride solution (15 mL). The solution was extracted with ethyl acetate (3×30 mL). The combined organic layers were dried over anhydrous magnesium sulfate, filtered, and concentrated under reduced pressure. The crude mixture was purified by flash column chromatography on silica gel (eluent: with 5% ethyl acetate in hexanes) to provide chloroalkyne 15′ (303 mg, 88%) as a colorless oil. Rf: 0.45 (10% ethyl acetate in hexanes). ¹H NMR (500 MHz, CDCl₃) δ 7.25 (d, J=8.5 Hz, 2H), 6.87 (d, J=8.7 Hz, 2H), 4.44 (s, 2H), 3.79 (s, 3H), 3.66 (t, J=6.8 Hz, 2H), 2.38 (tt, J=14.5, 6.8 Hz, 2H). ¹³C NMR (126 MHz, CDCl₃) δ 159.5, 130.1, 129.5, 114.1, 113.5 (t, J=234.1 Hz), 73.1, 68.3 (t, J=8.1 Hz), 63.9, 62.8 (t, J=42.2 Hz), 55.5, 39.7 (t, J=26.0 Hz). ¹⁹F NMR (471 MHz, CDCl₃) δ −80.9. FTIR (thin film) cm⁻¹: 2937 (w), 2870 (w), 2236 (m), 1614 (m), 1513 (s), 1245 (s), 1096 (s), 1033 (s), 820 (s). HIRMS (ESI) (m/z): calc'd for C₁₃H₁₃ClF₂NaO₂ [M+Na]⁺: 297.0464, found: 297.0463.

### Example 42: Synthesis of (Z)-1-chloro-N,N-diethyl-3,3-difluoro-5-((4-methoxybenzyl)oxy)pent-1-en-1-amine oxide (S5′)

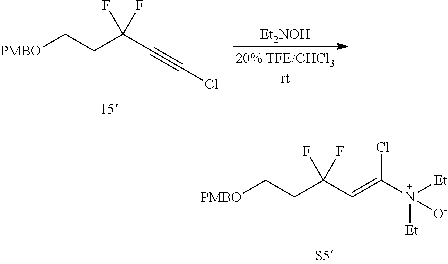

N,N-Diethylhydroxylamine (20.6 μL, 200 μmol) was added to a solution of chloroalkyne 15′ (27.5 mg, 100 μmol) in 20% 2,2,2-trifluoroethanol/chloroform (v/v, 0.5 mL). The reaction mixture stirred at room temperature for 3 hours. Upon completion of the reaction as determined by TLC, the reaction mixture was diluted with chloroform and purified by flash column chromatography on silica gel (eluent: 20% CMA in chloroform) to provide enamine N-oxide S5′ (36.9 mg, quantitative) as a colorless oil. Rf: 0.33 (40% CMA in chloroform). ¹H NMR (500 MHz, CD₃OD) δ 7.44 (t, J=12.3 Hz, 1H), 7.26 (d, J=8.6 Hz, 2H), 6.89 (d, J=8.7 Hz, 2H), 4.43 (s, 2H), 3.92-3.73 (m, 5H), 3.61 (t, J=6.3 Hz, 2H), 3.36 (dq, J=12.4, 7.2 Hz, 2H), 2.43 (tt, J=15.4, 6.3 Hz, 2H), 1.19 (t, J=7.1 Hz, 6H). ¹³C NMR (126 MHz, CD₃OD) δ 161.1, 141.6, 131.4, 130.8, 126.7 (t, J=31.1 Hz), 121.5 (t, J=240.0 Hz), 114.9, 74.0, 64.9, 64.4 (t, J=5.9 Hz), 55.8, 38.6 (t, J=25.8 Hz), 8.2. ¹⁹F NMR (471 MHz, CD₃OD) δ −92.5. FTIR (thin film) cm⁻¹: 3332 (br), 3063 (w), 2941 (w), 2874 (w), 1670 (m), 1610 (m), 1513 (s), 1245 (s), 1103 (s), 1029 (s), 813 (s). HRMS (ESI) (m/z): calc'd for C₁₇H₂₅ClF₂NO₃ [M+H]⁺: 364.1486, found: 364.1481.

### Example 43: Synthesis of (3,3-difluoro-3-(2-((4-methoxybenzyl)oxy)ethoxy)prop-1-yn-1-yl)triisopropylsilane (19′)

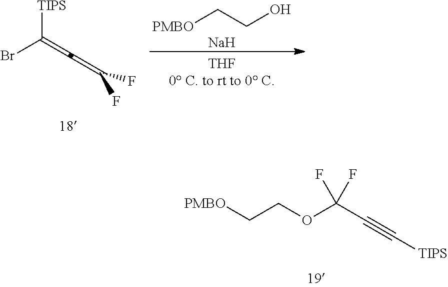

Sodium hydride (228 mg, 9.48 mmol) was added to a solution of allene 18′ (1.23 g, 3.95 mmol) (Xu et al., Angew. Chem., Int. Ed. 44(45):7404-7407 (2005)) in THE (30 mL) at 0° C. The ice-bath was immediately removed, and the reaction mixture stirred at room temperature for 1 hour. The solution was then cooled to 0° C. in a water-ice bath, and a solution of 2-((4-methoxybenzyl)oxy)ethan-1-ol (865 mg, 4.74 mmol) in THE (10 mL) was added dropwise via cannula. After 2 hours, the reaction mixture was quenched with saturated aqueous ammonium chloride solution (1 mL), diluted with diethyl ether (50 mL), and washed sequentially with water (50 mL) and brine (50 mL). The resulting organic layer was dried over anhydrous magnesium sulfate, filtered, and concentrated under reduced pressure. The crude mixture was purified by flash column chromatography on silica gel (eluent: 1% ethyl acetate in hexanes) to provide alkyne 19′ (1.37 g, 84%) as a colorless oil. Rf: 0.39 (5% ethyl acetate in hexanes). ¹H NMR (500 MHz, CDCl₃) δ 7.26 (d, J=8.6 Hz, 2H), 6.87 (d, J=8.7 Hz, 2H), 4.51 (s, 2H), 4.10-4.00 (m, 2H), 3.79 (s, 3H), 3.70-3.58 (m, 2H), 1.09 (d, J=4.1 Hz, 21H). ¹³C NMR (126 MHz, CDCl₃) δ 159.5, 130.2, 129.5, 114.0, 113.8 (t, J=242.7 Hz), 95.5 (t, J=51.8 Hz), 88.7 (t, J=5.2 Hz), 73.1, 67.7, 65.2 (t, J=3.2 Hz), 55.4, 18.6, 11.1. ¹⁹F NMR (471 MHz, CDCl₃) δ −54.8. FTIR (thin film) cm⁻¹: 2945 (m), 2866 (s), 1610 (w), 1513 (m), 1249 (s), 1263 (s), 1021 (s), 883 (m), 816 (m). HRMS (ESI) (m/z): calc'd for C₂₂H₃₄F₂NaO₃Si [M+Na]⁺: 435.2137, found: 435.2132.

### Example 44: Synthesis of 1-((2-((1,1-difluoroprop-2-yn-1-yl)oxy)ethoxy)methyl)-4-methoxybenzene (11′)

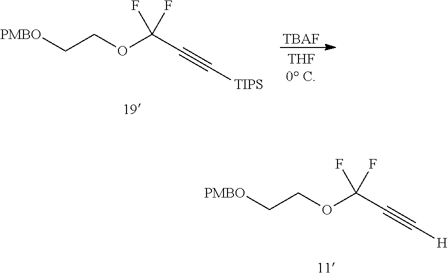

Tetrabutylammonium fluoride (TBAF, 1.70 mL, 1.70 mmol, 1 M in THF) was added dropwise via syringe to a solution of alkyne 19′ (700 mg, 1.70 mmol) in THE (15 mL) at 0° C. After 45 minutes, the reaction was quenched with saturated aqueous ammonium chloride solution (1 mL), diluted with diethyl ether (50 mL), and washed sequentially with water (50 mL) and brine (50 mL). The resulting organic layer was dried over anhydrous magnesium sulfate, filtered, and concentrated under reduced pressure. The residue was purified by flash column chromatography on silica gel (eluent: 7% ethyl acetate in hexanes) to provide alkyne 11′ (310 mg, 71%) as a colorless oil. Rf: 0.32 (10% ethyl acetate in hexanes). ¹H NMR (500 MHz, CDCl₃) δ 7.26 (d, J=8.5 Hz, 2H), 6.87 (d, J=8.6 Hz, 2H), 4.50 (s, 2H), 4.05-4.03 (m, 2H), 3.79 (s, 3H), 3.71-3.59 (m, 2H), 2.72 (t, J=3.4 Hz, 1H). ¹³C NMR (126 MHz, CDCl₃) δ 159.5, 130.1, 129.6, 114.0, 113.8 (t, J=244.0 Hz), 73.7, 73.4 (t, J=6.2 Hz), 73.1, 67.6, 65.0 (t, J=3.9 Hz), 55.5. ¹⁹F NMR (471 MHz, CDCl₃) δ −56.3. FTIR (thin film) cm⁻¹: 3273 (w), 2960 (w), 2904 (w), 2140 (m), 1614 (m), 1513 (s), 1249 (s), 1163 (s), 1103 (s), 1029 (s), 816 (m). HRMS (ESI) (m/z): calc'd for C₁₃H₁₄F₂NaO₃ [M+Na]⁺: 279.0803, found: 279.0802.

### Example 45: Synthesis of (E)-N,N-diethyl-3,3-difluoro-3-(2-((4-methoxybenzyl)oxy)ethoxy)prop-1-en-1-amine oxide (S6′)

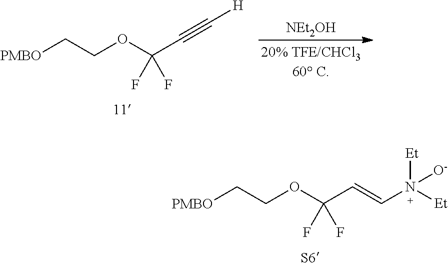

N,N-Diethylhydroxylamine (20.5 μL, 200 μmol) was added to a solution of alkyne 11′ (25.6 mg, 100 μmol) in 20% 2,2,2-trifluoroethanol/chloroform (v/v, 0.5 mL). The reaction mixture was then heated to 60° C. and stirred for 40 minutes. Upon completion of the reaction as determined by TLC, the reaction mixture was diluted with chloroform and purified by flash column chromatography on silica gel (eluent: 20% CMA in chloroform) to provide enamine N-oxide S6′ (37.4 mg, 100%) as a colorless oil. Rf: 0.23 (30% CMA in chloroform). ¹H NMR (500 MHz, CD₃OD) δ 7.27 (d, J=8.7 Hz, 2H), 6.94-6.85 (m, 3H), 6.62 (dt, J=13.1, 6.0 Hz, 1H), 4.49 (s, 2H), 4.12-4.05 (m, 2H), 3.78 (s, 3H), 3.71-3.67 (m, 2H), 3.48-3.36 (m, 4H), 1.25 (t, J=7.2 Hz, 6H). ¹³C NMR (126 MHz, CD₃OD) δ 161.1, 146.0 (t, J=6.9 Hz), 131.4, 130.7, 123.7 (t, J=36.5 Hz), 122.7 (t, J=254.7 Hz), 114.9, 73.9, 69.2, 65.5, 64.8 (t, J=5.6 Hz), 55.8, 8.5. ¹⁹F NMR (471 MHz, CD₃OD) δ −69.9. FTIR (thin film) cm⁻¹: 3320 (br), 3083 (w), 2945 (w), 2870 (w), 1700 (w), 1610 (m) 1513 (s), 1312 (s), 1249 (s), 1103 (s), 1029 (s), 977 (m). HRMS (ESI) (m/z): calc'd for C₁₇H₂₆F₂NO₄ [M+H]⁺: 346.1824, found: 346.1821.

### Example 46: Synthesis of 1-((2-((1,1-difluoro-3-iodoprop-2-yn-1-yl)oxy)ethoxy)methyl)-4-methoxybenzene (12′)

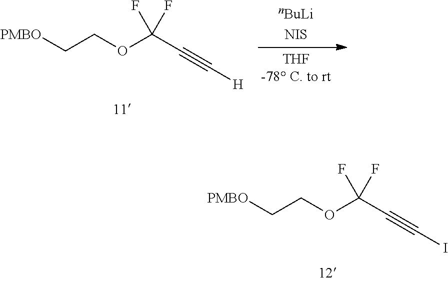

n-Butyllithium (131 μL, 328 μmol, 2.5 M in hexanes) was added dropwise via syringe to a solution of alkyne 11′ (70.0 mg, 273 μmol) in THE (4 mL) at −78° C. After 1 hour, N-iodosuccinimide (92.1 mg, 410 μmol) was added, the dry ice bath was removed, and the solution was allowed to warm to room temperature. After 1 hour, the reaction was quenched with saturated aqueous ammonium chloride solution (1 mL), diluted with diethyl ether (30 mL), washed with water (30 mL). The resulting organic layer was dried over anhydrous magnesium sulfate, filtered, and concentrated under reduced pressure. The crude mixture was purified by flash column chromatography on silica gel (eluent: 5% ethyl acetate in hexanes) to provide iodoalkyne 12′ (70.2 mg, 68%) as a colorless oil. Rf: 0.25 (10% ethyl acetate in hexanes). ¹H NMR (500 MHz, CDCl₃) δ 7.25 (d, J=8.6 Hz, 2H), 6.87 (d, J=8.6 Hz, 2H), 4.49 (s, 2H), 4.06-3.98 (m, 2H), 3.79 (s, 3H), 3.67-3.60 (m, 2H). ¹³C NMR (126 MHz, CDCl₃) δ 159.5, 130.0, 129.6, 114.1, 113.7 (t, J=245.0 Hz), 85.0 (t, J=54.5 Hz), 73.1, 67.6, 65.0 (t, J=3.8 Hz), 55.5, 9.8 (t, J=8.0 Hz). ¹⁹F NMR (471 MHz, CDCl₃) δ −55.3. FTIR (thin film) cm⁻¹: 2915 (w), 2859 (w), 2195 (w), 1610 (w) 1513 (m), 1252 (s), 1163 (s), 1100 (s), 1025 (s), 813 (m). HRMS (ESI) (m/z): calc'd for C₁₃H₁₃F₂IO₃ [M]: 381.9872, found: 381.9872.

### Example 47: Synthesis of (Z)-N,N-diethyl-3,3-difluoro-1-iodo-3-(2-((4-methoxybenzyl)oxy)ethoxy)prop-1-en-1-amine oxide (S7)

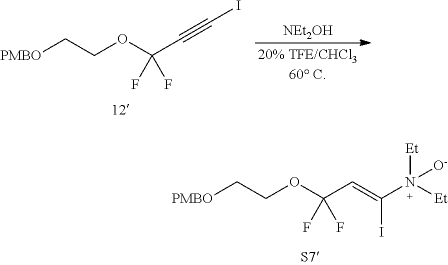

N,N-Diethylhydroxylamine (16.4 μL, 159 μmol) was added to a solution of iodoalkyne 12′ (30.4 mg, 79.5 μmol) in 20% 2,2,2-trifluoroethanol/chloroform (v/v 0.4 mL). The reaction mixture was then heated to 60° C. and stirred for 30 minutes. Upon completion of the reaction as determined by TLC, the reaction mixture was diluted with chloroform and purified by flash column chromatography on silica gel (eluent: 10% CMA in chloroform) to provide enamine N-oxide S7′ (37.5 mg, 99%) as a white solid. Rf: 0.16 (30% CMA in chloroform). ¹H NMR (500 MHz, CD₃OD) δ 7.92 (t, J=7.5 Hz, 1H), 7.27 (d, J=8.6 Hz, 2H), 6.89 (d, J=8.6 Hz, 2H), 4.50 (s, 2H), 4.13-4.07 (m, 2H), 3.93 (dq, J=12.3, 7.0 Hz, 2H), 3.78 (s, 3H), 3.75-3.69 (m, 2H), 3.40-3.33 (m, 2H), 1.18 (t, J=7.0 Hz, 6H). ¹³C NMR (126 MHz, CD₃OD) δ 161.0, 134.4 (t, J=38.5 Hz), 131.5, 130.7, 122.4 (t, J=256.1 Hz), 114.9, 112.8 (t, J=5.3 Hz), 73.9, 69.0, 65.9, 64.8 (t, J=5.5 Hz), 55.8, 8.1. ¹⁹F NMR (471 MHz, CD₃OD) δ −67.2. FTIR (thin film) cm⁻¹: 3176 (br), 3649 (w), 2926 (s), 2855 (m), 1610 (m), 1513 (s), 1297 (s), 1245 (s), 1100 (s), 1029 (s), 820 (m). HRMS (ESI) (m/z): calc'd for C₁₇H₂₅F₂INO₄ [M+H]⁺: 472.0791, found: 472.0786.

### Example 48: Synthesis of 1-((2-((3-bromo-1,1-difluoroprop-2-yn-1-yl)oxy)ethoxy)methyl)-4-methoxybenzene (13′)

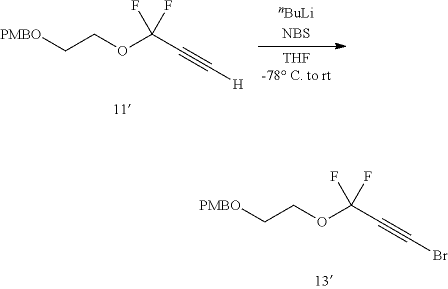

n-Butyllithium (93.6 μL, 234 μmol, 2.5 M in hexanes) was added dropwise via syringe to a solution of alkyne 11′ (50.0 mg, 195 μmol) in THE (2 mL) at −78° C. After 1 hour, N-bromosuccinimide (NBS, 52.2 mg, 293 μmol) was added, the dry ice bath was removed, and the solution was allowed to warm to room temperature. After 1 hour, the reaction was quenched with saturated aqueous ammonium chloride solution (1 mL), diluted with diethyl ether (30 mL), and washed with water (30 mL). The resulting organic layer was dried over anhydrous magnesium sulfate, filtered, and concentrated under reduced pressure. The crude mixture was purified by flash column chromatography on silica gel (eluent: 5% ethyl acetate in hexanes) to provide bromoalkyne 13′ (41.1 mg, 61%) as a colorless oil. Rf: 0.32 (7% ethyl acetate in hexanes). ¹H NMR (500 MHz, CDCl₃) δ 7.25 (d, J=8.6 Hz, 2H), 6.87 (d, J=8.7 Hz, 2H), 4.50 (s, 2H), 4.04-4.01 (m, 2H), 3.79 (s, 3H), 3.65-3.62 (m, 2H). ¹³C NMR (126 MHz, CDCl₃) δ 159.5, 130.0, 129.6, 114.1 (t, J=244.5 Hz), 114.0, 73.1, 71.1 (t, J=55.3 Hz), 67.6, 65.0, 55.4, 50.1 (t, J=7.6 Hz). ¹⁹F NMR (471 MHz, CDCl₃) δ −55.2. FTIR (thin film) cm⁻¹: 2956 (w), 2903 (w), 2866 (w), 2229 (m), 1610 (m), 1513 (s), 1267 (s), 1162 (s), 1103 (s), 1029 (s), 820 (m). HRMS (ESI) (m/z): calc'd for C₁₃H₁₃BrF₂NaO₃ [M+Na]⁺: 356.9908, found: 356.9906.

### Example 49: Synthesis of (Z)-1-bromo-N,N-diethyl-3,3-difluoro-3-(2-((4-methoxybenzyl)oxy)ethoxy)prop-1-en-1-amine oxide (S8′)

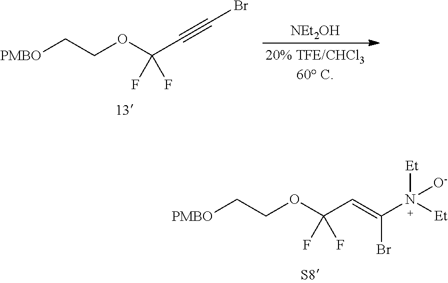

N,N-Diethylhydroxylamine (15.3 μL, 149 μmol) was added to a solution of bromoalkyne 13′ (25.0 mg, 74.6 μmol) in 20% 2,2,2-trifluoroethanol/chloroform (v/v, 375 μL). The reaction mixture was then heated to 60° C. and stirred for 15 minutes. Upon completion of the reaction as determined by TLC, the reaction mixture was diluted with chloroform and purified by flash column chromatography on silica gel (eluent: 20% CMA in chloroform) to provide enamine N-oxide S8′ (30.3 mg, 95%) as a white solid. Rf: 0.13 (30% CMA in chloroform). ¹H NMR (500 MHz, CD₃OD) δ 7.74 (t, J=7.4 Hz, 1H), 7.27 (d, J=8.6 Hz, 2H), 6.89 (d, J=8.6 Hz, 2H), 4.50 (s, 2H), 4.15-4.08 (m, 2H), 3.90 (dq, J=12.3, 7.0 Hz, 2H), 3.78 (s, 3H), 3.72-3.67 (m, 2H), 3.39 (dq, J=12.4, 7.2 Hz, 2H), 1.22 (t, J=7.1 Hz, 6H). ¹³C NMR (126 MHz, CD₃OD) δ 161.0, 135.8, 131.5, 130.7, 128.0 (t, J=38.5 Hz), 122.4 (t, J=256.4 Hz), 114.9, 73.9, 69.0, 65.5, 64.9 (t, J=5.5 Hz), 55.8, 8.1. ¹⁹F NMR (471 MHz, CD₃OD) δ −67.8. FTIR (thin film) cm⁻¹: 3220 (br), 3064 (w), 2941 (w), 2855 (w), 1662 (m), 1610 (m), 1513 (m), 1301 (s), 1249 (s), 1167 (m), 1103 (s), 1029 (s), 820 (m). HRMS (ESI) (m/z): calc'd for C₁₇H₂₅BrF₂NO₄ [M+H]⁺: 424.0930, found: 424.0924.

### Example 50: Synthesis of 1-((2-((3-chloro-1,1-difluoroprop-2-yn-1-yl)oxy)ethoxy)methyl)-4-methoxybenzene (10′)

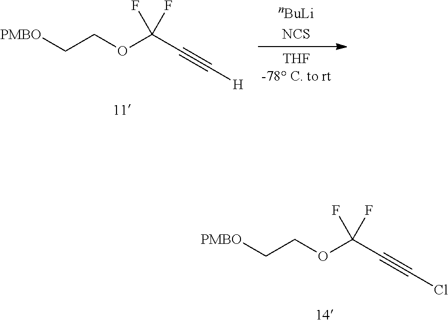

n-Butyllithium (150 μL, 374 μmol, 2.5 M in hexanes) was added dropwise via syringe to a solution of 11′ (80.0 mg, 312 μmol) in THF (4 ml) at −78° C. After 30 minutes, N-chlorosuccinimide (62.5 mg, 468 μmol) was added, the dry ice bath was removed, and the solution was allowed to warm to room temperature. After 1 hour and 45 minutes, the reaction was quenched with saturated aqueous ammonium chloride solution (1 mL), diluted with diethyl ether (30 mL), and washed with water (30 mL). The resulting organic layer was dried over anhydrous magnesium sulfate, filtered, and concentrated under reduced pressure. The crude mixture was purified by flash column chromatography on silica gel (eluent: 5% ethyl acetate in hexanes) to provide chloroalkyne 14′ (85.6 mg, 94%) as a colorless oil. Rf: 0.40 (10% ethyl acetate in hexanes). ¹H NMR (500 MHz, CDCl₃) δ 7.26 (d, J=8.7 Hz, 2H), 6.87 (d, J=8.6 Hz, 2H), 4.50 (s, 2H), 4.08-3.97 (m, 2H), 3.79 (s, 3H), 3.70-3.56 (m, 2H). ¹³C NMR (126 MHz, CD₃OD) δ 161.0, 131.3, 130.7, 115.5 (t, J=242.6 Hz), 114.9, 73.9, 68.8, 67.7 (t, J=7.3 Hz), 66.4 (t, J=3.8 Hz), 61.1 (t, J=56.5 Hz), 55.8. ¹⁹F NMR (471 MHz, CDCl₃) δ −54.8. FTIR (thin film) cm⁻¹: 2960 (w), 2866 (w), 2248 (m), 1614 (m), 1513 (s), 1282 (s), 1249 (s), 1170 (s), 1107 (s), 1033 (s). HRMS (ESI) (m/z): calc'd for C₁₃H₁₃ClF₂NaO₃ [M+Na]⁺: 313.0413, found: 313.0413.

### Example 51: Synthesis of (Z)-1-chloro-N,N-diethyl-3,3-difluoro-3-(2-((4-methoxybenzyl)oxy)ethoxy)prop-1-en-1-amine oxide (20′)

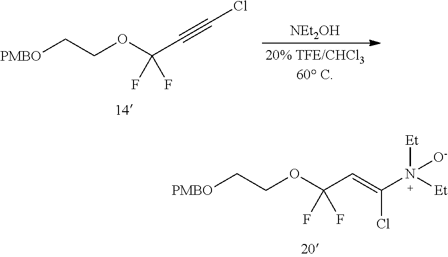

N,N-Diethylhydroxylamine (20.5 μL, 200 μmol) was added to a solution of chloroalkyne 14′ (29.1 mg, 100 μmol) in 20% trifluoroethanol/chloroform (v/v, 0.5 mL). The reaction mixture was then heated to 60° C. and stirred for 10 minutes. Upon completion of the reaction as determined by TLC, the reaction mixture was diluted with chloroform and purified by flash column chromatography on silica gel (eluent: 10% CMA in chloroform) to provide enamine N-oxide 20′ (16.2 mg, 43%) as a white solid. Rf: 0.33 (30% CMA in chloroform). ¹H NMR (500 MHz, CD₃OD) δ 7.43 (t, J=7.4 Hz, 1H), 7.26 (d, J=8.7 Hz, 2H), 6.89 (d, J=8.7 Hz, 2H), 4.49 (s, 2H), 4.19-4.06 (m, 2H), 3.84 (dq, J=12.3, 7.0 Hz, 2H), 3.78 (s, 3H), 3.71-3.67 (m, 2H), 3.41 (dq, J=12.3, 7.2 Hz, 2H), 1.23 (t, J=7.1 Hz, 6H). ¹³C NMR (126 MHz, CD₃OD) δ 161.0, 144.6, 131.5, 130.6, 124.0 (t, J=38.2 Hz), 122.3 (t, J=256.4 Hz), 114.9, 73.9, 69.0, 65.0, 64.9 (t, J=5.5 Hz), 55.8, 8.1. ¹⁹F NMR (471 MHz, CD₃OD) δ −68.0. FTIR (thin film) cm⁻¹: 3332 (br), 3079 (w), 2941 (W), 2859 (w), 1674 (w), 1610 (w), 1513 (m), 1305 (s), 1245 (s), 1170 (m), 1103 (s), 1029 (s), 816 (m). HRMS (ESI) (m/z): calc'd for C₁₇H₂₅ClF₂NO₄ [M+H]⁺: 380.1435, found: 380.1432.

### Example 52: Synthesis of 2-((3-chloro-1,1-difluoroprop-2-yn-1-yl)oxy)ethan-1-ol (S9′)

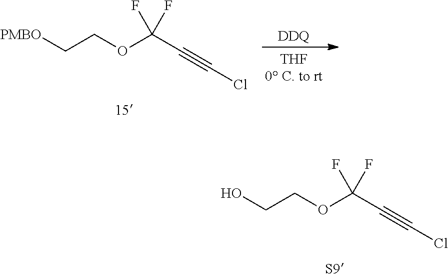

2,3-Dichloro-5,6-dicyano-p-benzoquinone (DDQ, 71.3 mg, 314 μmol) was added to a solution of chloroalkyne 15′ (83.0 mg, 285 μmol) in 5% water/DCM (v/v, 3.15 mL) at 0° C. After 1 hour, the ice bath was removed, and the solution was allowed to warm to room temperature. After 2 hours, upon completion of the reaction as determined by TLC, the reaction mixture was diluted with diethyl ether (30 mL) and washed sequentially with water (30 mL) and brine (30 mL). The resulting organic layer was dried over anhydrous magnesium sulfate, filtered, and concentrated under reduced pressure. The residue was purified by flash column chromatography on silica gel (eluent: 20% diethyl ether in pentane) to provide alcohol S9′ (44.9 mg, 91%) as a colorless oil. Trace amounts of diethyl ether and pentane could not be removed completely due to the volatility of the compound and were present in the ¹H and ¹³C NMR. Rf: 0.17 (30% diethyl ether in pentane). ¹H NMR (500 MHz, CDCl₃) δ 4.00 (t, J=4.3 Hz, 2H), 3.81 (t, J=4.5 Hz, 2H). ¹³C NMR (126 MHz, CDCl₃) δ 114.1 (t, J=244.8 Hz), 67.1 (t, J=3.3 Hz), 66.8 (t, J=7.2 Hz), 61.1, 60.2 (t, J=55.6 Hz). ¹⁹F NMR (471 MHz, CDCl₃) δ −54.7. FTIR (thin film) cm⁻¹: 3340 (br), 2960 (w), 2244 (m), 1279 (s), 1159 (s), 1029 (s), 936 (m). HRMS (ESI) (m/z): calc'd for C₅H₅ClF₂NaO₂ [M+Na]⁺: 192.9838, found: 192.9839.

### Example 53: Synthesis of 2-((3-chloro-1.1-difluoroprop-2-yn-1-yl)oxy)ethyl (4-nitrophenyl) carbonate (S10′)

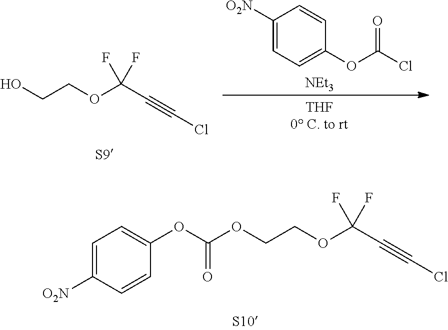 [SMILES: OCCOC(F)(F)C#CCl ; O=C(Cl)Oc1ccc([N+](=O)[O-])cc1 ; O=C(OCCOC(F)(F)C#CCl)Oc1ccc([N+](=O)[O-])cc1 ; "NEt3"]

Triethylamine (508 μmol, 70.9 μL) was added via syringe to a solution of chloroalkyne S9′ (42.0 mg, 254 μmol) in THE (2 mL) at 0° C. A solution of 4-nitrophenyl chloroformate (267 μmol, 53.8 mg) in THE (2 mL) was then added dropwise via cannula to the resulting solution. After 2 hours, the reaction was quenched by adding ice flakes, diluted with diethyl ether (30 mL), and sequentially washed with water (30 mL) and brine (30 mL). The resulting organic layer was dried over anhydrous magnesium sulfate, filtered, and concentrated under reduced pressure. The crude mixture was purified by flash column chromatography on silica gel (eluent: 40% DCM in hexanes) to provide carbonate S10′ (39.0 mg, 48%). Rf: 0.14 (40% DCM in hexanes). ¹H NMR (500 MHz, CDCl₃) δ 8.27 (d, J=9.1 Hz, 2H), 7.37 (d, J=9.1 Hz, 2H), 4.50-4.44 (m, 2H), 4.22-4.16 (m, 2H). ¹³C NMR (126 MHz, CDCl₃) δ 155.5, 152.5, 145.7, 125.6, 122.0, 113.9 (t, J=246.0 Hz), 67.1 (t, J=7.0 Hz), 66.7, 62.8 (t, J=4.3 Hz), 59.9 (t, J=54.6 Hz). ¹⁹F NMR (471 MHz, CDCl₃) δ −55.3. FTIR (thin film) cm⁻¹: 2244 (w), 1771 (m), 1666 (w), 1528 (m), 1349 (m), 1219 (s), 1163 (m). HRMS (ESI) (m/z): calc'd for C₁₂H₈ClF₂NNaO₆ [M+Na]⁺: 357.9900, found: 357.9895.

### Example 54: Synthesis of 2-((3-chloro-1,1-difluoroprop-2-yn-1-yl)oxy)ethyl (2-(2-((6-chlorohexyl)oxy)ethoxy)ethyl)carbamate (21′)

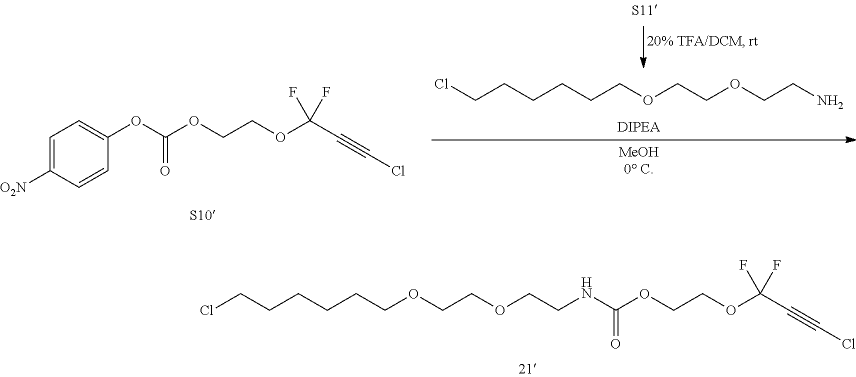 [SMILES: O=C(OCCOC(F)(F)C#CCl)Oc1ccc([N+](=O)[O-])cc1 ; NCCOCCOCCCCCCCl ; O=C(NCCOCCOCCCCCCCl)OCCOC(F)(F)C#CCl]

DCM (2.4 mL) and trifluoroacetic acid (0.6 mL) were sequentially added to a 10 mL pear-shaped flask charged with Boc-amine S11′ (Foley et al., ACS Chem. Biol. 15(1):290-295 (2020)) (33.8 mg, 104 μmol) at room temperature. After 30 minutes, the solution was concentrated under reduced pressure. The resulting amine was dissolved in methanol (0.5 mL) and N,N-diisopropylethylamine (32.6 μL, 187 μmol) was added via syringe. The solution was cooled to 0° C. in an ice-water bath, and a solution of nitrophenyl carbonate S10′ (31.8 mg, 93.7 μmol) was added to the solution. An additional portion of N,N-diisopropylethylamine (32.6 μL, 187 μmol) was added to the reaction mixture. After 6.5 hours, the reaction mixture was diluted with ethyl acetate (30 mL) and sequentially washed with water (30 mL) and brine (30 mL). The resulting organic layer was dried over anhydrous magnesium sulfate, filtered, and concentrated under reduced pressure. The residue was purified by flash column chromatography on silica gel (eluent: 35→40% ethyl acetate in hexanes) to afford the title compound (25.0 mg, 63%) as a colorless oil. Rf: 0.25 (30% ethyl acetate in hexanes). ¹H NMR (500 MHz, CDCl₃) δ 5.31 (s, 1H), 4.25 (t, J=4.7 Hz, 2H), 4.08-4.02 (m, 2H), 3.60-3.57 (m, 2H), 3.56-3.49 (m, 6H), 3.44 (t, J=6.7 Hz, 2H), 3.36 (q, J=5.3 Hz, 2H), 1.80-1.69 (m, 2H), 1.63-1.56 (m, 2H), 1.49-1.31 (m, 4H). ¹³C NMR (126 MHz, CDCl₃) δ 156.2, 114.0 (t, J=245.2 Hz), 71.5, 70.6, 70.2, 70.1, 66.7 (t, J=7.1 Hz), 64.0 (t, J=4.0 Hz), 62.6, 60.2 (t, J=55.2 Hz), 45.3, 41.1, 32.7, 29.6, 26.9, 25.6. ¹⁹F NMR (471 MHz, CDCl₃) δ −55.0. FTIR (thin film) cm⁻¹: 3340 (br), 2937 (m), 2863 (m), 2244 (m), 1722 (s), 1521 (m), 1279 (s), 1234 (s), 1141 (s), 1101 (s), 1029 (s), 943 (m). HRMS (ESI) (m/z): calc'd for C₁₆H₂₅Cl₂F₂NNaO₅ [M+Na]⁺: 442.0970, found: 442.0960.

### Example 55: Synthesis of tert-butyl (2-(2-(hydroxy(methyl)amino)ethoxy)ethyl)carbamate (S13′)

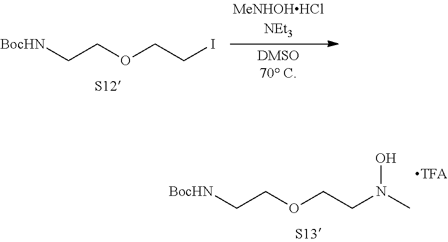 [SMILES: CC(C)(C)OC(=O)NCCOCCI ; CN(O)CCOCCNC(=O)OC(C)(C)C]

Triethylamine (1.34 mL, 9.59 mmol) was added to a solution of alkyl iodide S12′ (Heller et al., Angew. Chem., Int. Ed. 54(35):10327-10330 (2015)) (756 mg, 2.40 mmol) and N-methylhydroxylamine hydrochloride (401 mg, 4.80 mmol) in dimethyl sulfoxide (2.4 mL). The reaction mixture was then heated to 70° C. After 1.5 hours, the solution was cooled to room temperature, diluted with water, and purified by automated C₁₈ reverse phase column chromatography (30 g C₁₈ silica gel, 25 μm spherical particles, eluent: H₂O+0.1% TFA (5 CV), gradient 0→100% MeCN/H₂O+0.1% TFA (15 CV)) to provide hydroxylamine S13′ (348 mg, 62%) as a white solid. ¹H NMR (500 MHz, CDCl₃) δ 3.82 (ddd, J=11.1, 7.3, 3.9 Hz, 1H), 3.63 (dt, J=11.0, 4.2 Hz, 1H), 3.52-3.35 (m, 4H), 3.32-3.18 (m, 2H), 3.07 (s, 3H), 1.38 (s, 9H). ¹³C NMR (126 MHz, CDCl₃) δ 164.1 (q, J=37.5 Hz), 156.7, 116.5 (q, J=289.2 Hz), 79.5, 70.9, 63.6, 60.2, 46.5, 40.4, 28.5. ¹⁹F NMR (471 MHz, CDCl₃) δ −75.5. FTIR (thin film) cm⁻¹: 3351 (br), 2945 (w), 2900 (w), 2236 (s), 1361 (m), 1290 (m), 1185 (s), 1129 (s), 1085 (s). HRMS (ESI) (m/z): calc'd for C₁₀H₂₃N₂O₄ [M+H]⁺: 235.1652, found: 235.1650.

### Example 56: Synthesis of 3′,6′-bis(dimethylamino)-N-(2-(2-(hydroxy(methyl)amino)ethoxy)ethyl)-3-oxo-3H-spiro[isobenzofuran-1,9′-xanthene]-6-carboxamide (22′)

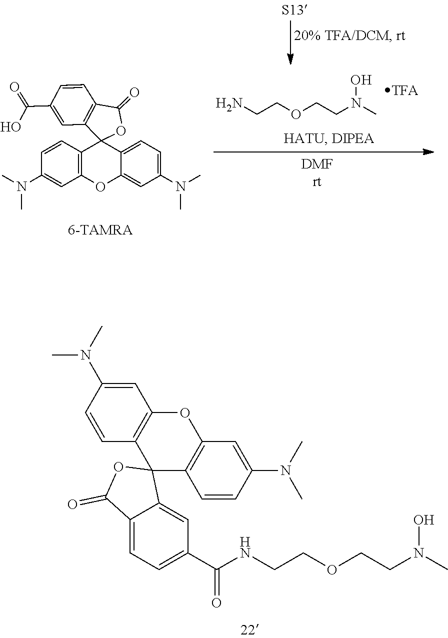

N,N-Diisopropylethylamine (49.2 μL, 282 μmol) was added to a solution of 6-carboxytetramethylrhodamine (6-TAMRA, 30.4 mg, 70.6 μmol) and 1-[bis(dimethylamino)methylene]-1H-1,2,3-triazolo[4,5-b]pyridinium 3-oxid hexafluorophosphate (HATU, 29.5 mg, 77.7 μmol) in N,N-dimethylformamide (0.7 mL) at room temperature. In a separate vial, trifluoroacetic acid (0.1 mL) was added to a solution of hydroxylamine S13′ (61.5 mg, 177 μmol) in DCM (0.4 mL) at room temperature. After 1 hour, the hydroxylamine-containing solution was concentrated under reduced pressure, redissolved in N,N-dimethylformamide (0.5 mL), and then added to the reaction solution containing 6-TAMRA using a pipette. An additional portion of N,N-dimethylformamide (0.2 mL) was used to quantitatively transfer the hydroxylamine. After 4 hours, additional portions of HATU (HATU, 29.5 mg, 77.7 μmol) and N,N-diisopropylethylamine (49.2 μL, 282 μmol) were sequentially added to the reaction mixture. After 2.5 hours, the resulting mixture was diluted with water and purified by automated C is reverse phase column chromatography (30 g C₁₈ silica gel, 25 μm spherical particles, eluent: H₂O+0.1% TFA (5 CV), gradient 0→100% MeCN/H₂O+0.1% TFA (15 CV)). The resulting residue was then purified by flash column chromatography on silica gel (eluent: 70% CMA in chloroform) to provide TAMRA-hydroxylamine 22′ (22.8 mg, 59%) as a violet solid. ¹H NMR (500 MHz, D₂O) δ 7.99 (d, J=8.3 Hz, 1H), 7.87 (d, J=8.1 Hz, 1H), 7.67-7.59 (m, 1H), 7.07 (d, J=9.5 Hz, 2H), 6.73 (dd, J=9.5, 2.4 Hz, 2H), 6.41 (d, J=2.3 Hz, 2H), 3.84-3.63 (m, 4H), 3.53 (t, J=5.4 Hz, 2H), 3.11-2.89 (m, 14H), 2.71 (s, 3H). ¹³C NMR (126 MHz, D₂O) δ 173.2, 168.0, 157.6, 156.7, 156.6, 143.0, 133.5, 130.9, 130.6, 129.1, 128.9, 128.1, 113.6, 112.7, 96.1, 68.8, 66.6, 60.2, 47.5, 34.0, 39.7. FTIR (thin film) cm⁻¹: 3280 (br), 2926 (w), 1648 (w) 1595 (s), 1491 (m), 1409 (m), 1349 (m), 1189 (m). HRMS (ESI) (m/z): calc'd for C₃₀H₃₅N₄O₆ [M+H]⁺: 547.2551, found: 547.2544.

### Example 57: Synthesis of di-tert-butyl (18-chloro-3,6,9,12-tetraoxaoctadecyl)iminodicarbonate (S14′)

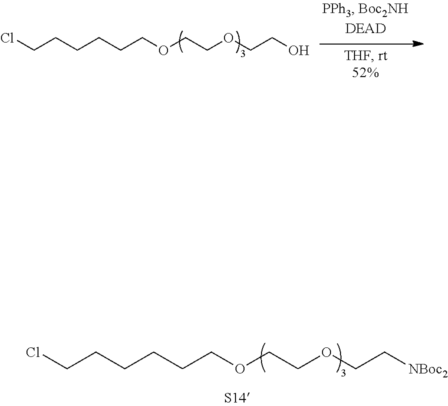

Diethyl azodicarboxylate (DEAD, 40% in toluene, 233 μL, 512 μmol) was added dropwise via syringe to a solution of 18-chloro-3,6,9,12-tetraoxaoctadecan-1-ol (80.0 mg, 256 μmol), triphenylphosphine (131 mg, 512 μmol), and di-tert-butyl iminodicarbonate (111 mg, 512 μmol) in THF (5 mL) at room temperature. After 2.5 hours, the reaction was concentrated under reduced pressure. The crude mixture was purified by flash column chromatography on silica gel (eluent: 20% acetone in hexanes) to provide chloroalkane S14′ (67.7 mg, 52%) as a colorless oil. n¹H NMR (500 MHz, CDCl₃) δ 3.74 (t, J=6.3 Hz, 2H), 3.63-3.51 (m, 14H), 3.48 (t, J=6.7 Hz, 2H), 3.41 (t, J=6.6 Hz, 2H), 1.76-1.68 (m, 2H), 1.59-1.50 (m, 2H), 1.45 (s, 18H), 1.43-1.28 (m, 4H). ¹³C NMR (126 MHz, CDCl₃) δ 152.8, 82.4, 71.4, 70.8, 70.8, 70.7, 70.7, 70.4, 70.2, 69.4, 45.3, 45.2, 32.7, 29.6, 28.2, 26.8, 25.6. FTIR (thin film) cm⁻¹: 2863 (w), 1696 (m), 1349 (m), 1118 (s), 854 (w). HRMS (ESI) (m/z): calc'd for C₂₄H₄₆ClNNaO₈[M+Na]⁺: 534.2804, found: 534.2811.

### Example 58: Synthesis of 2-((3-chloro-1,1-difluoroprop-2-yn-1-yl)oxy)ethyl (18-chloro-3,6,9,12-tetraoxaoctadecyl)carbamate (S15)

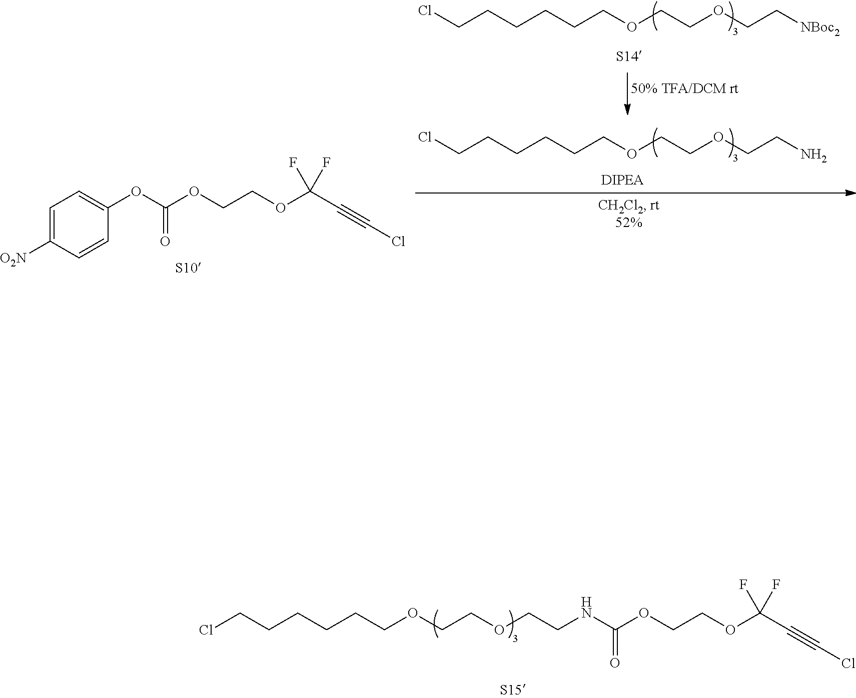

Trifluoroacetic acid (TFA, 200 μL) was added via syringe to a solution of chloroalkane S14′ (36.6 mg, 71.5 μmol) in DCM (200 μL) at room temperature. After 1 hour, the reaction was concentrated under reduced pressure then diluted with DCM (2.5 mL). N,N-Diisopropylethylamine (DIPEA, 51.9 μL, 298 μmol) and nitrophenyl carbonate S10′ (20.0 mg, 59.6 μmol) were sequentially added to the solution at room temperature. After 17 hours, the reaction mixture was purified by flash column chromatography on silica gel (eluent: 50% ethyl acetate in hexanes) to provide HaloTag ligand S15′ (15.7 mg, 52%) as a colorless oil. ¹H NMR (500 MHz, CDCl₃) δ 5.45 (s, 1H), 4.34-4.19 (m, 2H), 4.10-3.99 (m, 2H), 3.66-3.58 (m, 10H), 3.57-3.48 (m, 6H), 3.43 (t, J=6.7 Hz, 2H), 3.35 (q, J=5.4 Hz, 2H), 1.79-1.71 (m, 2H), 1.61-1.53 (m, 2H), 1.48-1.30 (m, 4H). ¹³C NMR (126 MHz, CDCl₃) δ 156.3, 113.96 (t, J=244.8 Hz), 71.4, 70.8, 70.8, 70.8, 70.8, 70.5, 70.3, 70.2, 66.7, 64.05 (t, J=4.1 Hz), 62.6, 60.19 (t, J=55.5 Hz), 45.3, 41.1, 32.8, 29.7, 26.9, 25.6. ¹⁹F NMR (471 MHz, CDCl₃) δ −55.0. FTIR (thin film) cm⁻¹: 2922 (m), 2862 (m), 2244 (w), 1722 (s), 1528 (m), 1103 (s). HRMS (ESI) (m/z): calc'd for C₂₀H₃₄C₁₂F₂NO₇ [M+H]⁺: 508.1675, found: 508.1677.

### Example 59: Reactivity Screening

Each experiment was performed with 10 mM alkyne (8′-15′) and 50 mM hydroxylamine 22′ in CD₃CN at room temperature. The consumption of starting material was monitored via ¹H (8′) or ¹⁹F (9′-15′) NMR spectroscopy using α,α,α-benzotrifluoride as an internal standard (FIG. 12A).

### Example 60: Kinetics Study

All kinetics experiments were carried out at room temperature in CD₃CN. Reactions were monitored via ¹⁹F NMR spectroscopy using α,α,α-benzotrifluoride as an internal standard. Pseudo first-order kinetics studies were performed for compounds 11′ and 12′ using 2 mM alkyne and varying concentrations of N,N-diethylhydroxylamine (2′). Second-order kinetics were performed by combining alkynes 13′-14′ and hydroxylamine 2′ in a 1:1 molar ratio to achieve a final concentration of 10 mM for each component. The reported errors for rate constants represent the standard deviation of the mean for triplicate experiments (FIG. 13).

### Example 61: Stability Study

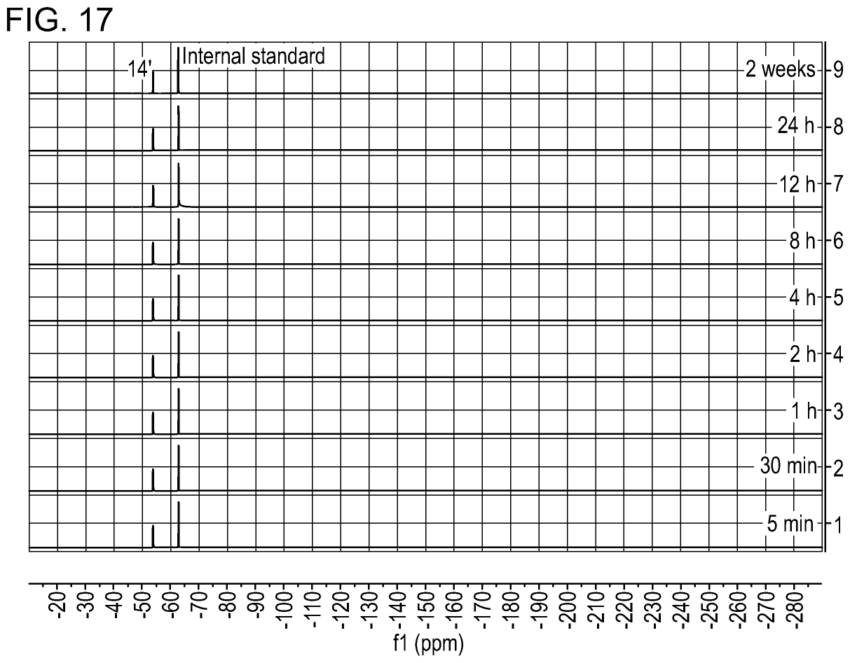

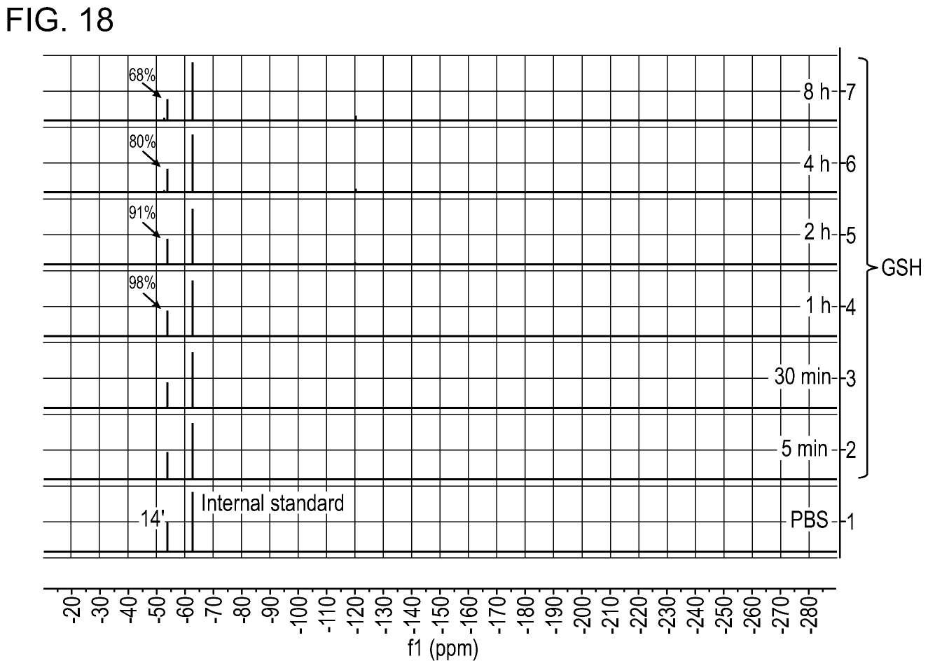

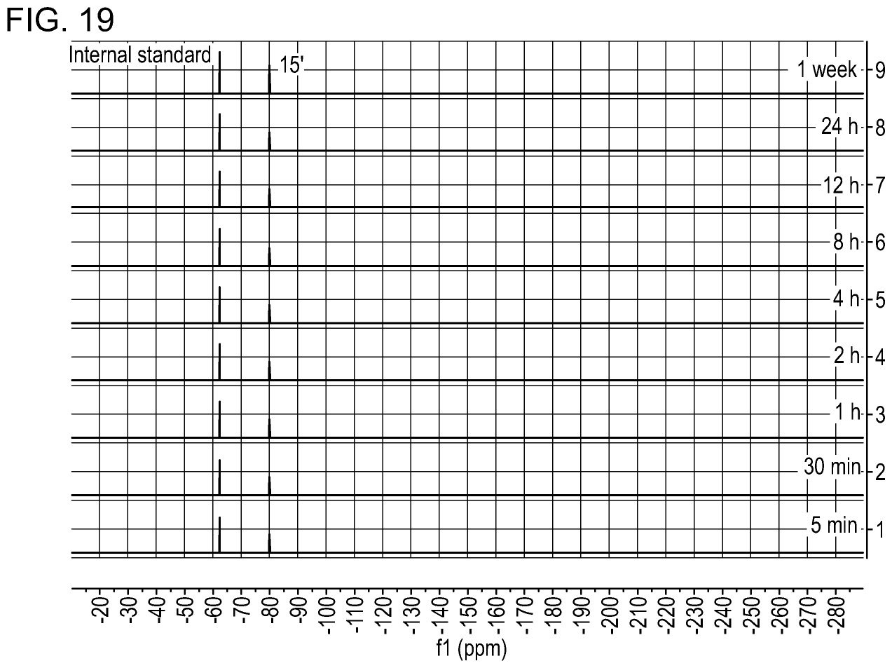

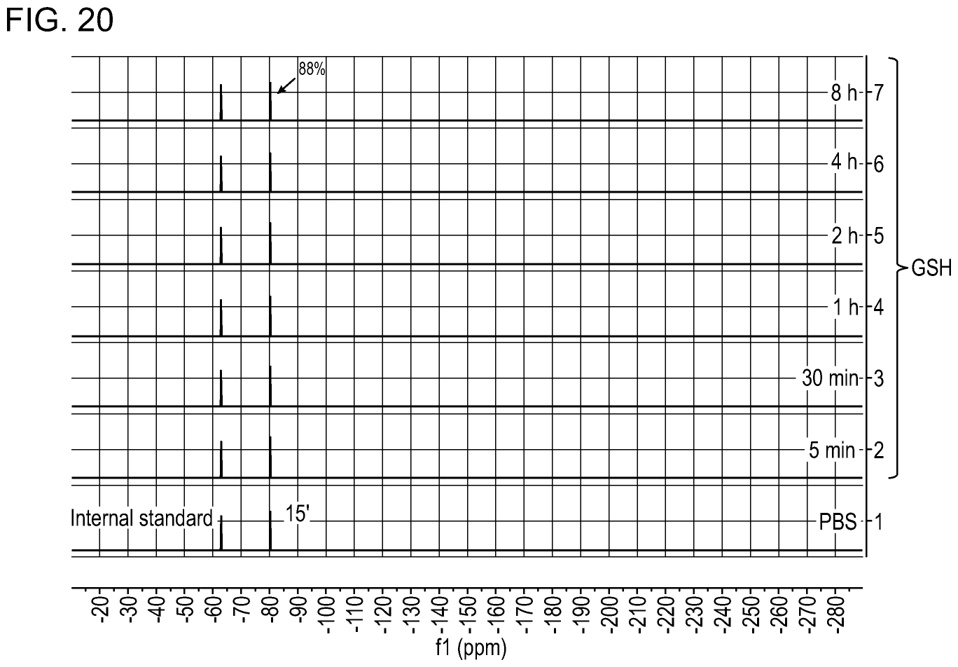

All stability experiments were carried out at room temperature in 50% CD₃CN in PBS (pH 7.0) with or without equimolar glutathione. pH was adjusted after the addition of glutathione. Reactions were monitored via ¹⁹F NMR spectroscopy using α,α,α-benzotrifluoride as an internal standard (FIG. 17-FIG. 20).

### Example 62: HaloTag-His₆ Protein Expression and Purification

pET28b-HaloTag-His6 gene (SEQ ID NO: 1):
. . . AATAATTTTGTTTAACTTTAAGAAGGAGATATACCCTCGAGATG
GGATCCGAAATCGGTACTGGCTTTCCATTCGACCCCCATTATGTGGAAGT
CCTGGGCGAGCGCATGCACTACGTCGATGTTGGTCCGCGCGATGGCACCC
CTGTGCTGTTCCTGCACGGTAACCCGACCTCCTCCTACGTGTGGCGCAAC
ATCATCCCGCATGTTGCACCGACCCATCGCTGCATTGCTCCAGACCTGAT
CGGTATGGGCAAATCCGACAAACCAGACCTGGGTTATTTCTTCGACGACC
ACGTCCGCTTCATGGATGCCTTCATCGAAGCCCTGGGTCTGGAAGAGGTC
GTCCTGGTCATTCACGACTGGGGCTCCGCTCTGGGTTTCCACTGGGCCAA
GCGCAATCCAGAGCGCGTCAAAGGTATTGCATTTATGGAGTTCATCCGCC
CTATCCCGACCTGGGACGAATGGCCAGAATTTGCCCGCGAGACCTTCCAG
GCCTTCCGCACCACCGACGTCGGCCGCAAGCTGATCATCGATCAGAACGT
TTTTATCGAGGGTACGCTGCCGATGGGTGTCGTCCGCCCGCTGACTGAAG
TCGAGATGGACCATTACCGCGAGCCGTTCCTGAATCCTGTTGACCGCGAG
CCACTGTGGCGCTTCCCAAACGAGCTGCCAATCGCCGGTGAGCCAGCGAA
CATCGTCGCGCTGGTCGAAGAATACATGGACTGGCTGCACCAGTCCCCTG
TCCCGAAGCTGCTGTTCTGGGGCACCCCAGGCGTTCTGATCCCACCGGCC
GAAGCCGCTCGCCTGGCCAAAAGCCTGCCTAACTGCAAGGCTGTGGACAT
CGGCCCGGGTCTGAATCTGCTGCAAGAAGACAACCCGGACCTGATCGGCA
GCGAGATCGCGCGCTGGCTGTCTACTCTGGAGATTTCCGGTGAGCACCAC
CACCACCACCACTGAGATCCGGCTGCTAACA . . .

Cloning for pET28b-HaloTag-His₆: HaloTag sequence (underlined) was obtained from pHTC CMV-neo vector (Promega™) and inserted into a pET28b vector. The coding region is bolded.

pET28b-HaloTag-His₆ was transformed into chemically competent DH5a cells and selected on kanamycin LB/agar plates. A single colony was selected and used to inoculate LB media (200 mL) containing 50 μg/mL kanamycin. The starter culture was grown to saturation overnight. 4×1 L LB broth with 50 μg/mL kanamycin were inoculated with the starter culture (50 mL) and grown to OD₆₀₀ ˜0.8. Protein expression was induced with 0.2 mM IPTG, and the culture was incubated for 3 hours at 37° C. The cells were pelleted by centrifugation (20 min, 7000×g) at 4° C. Lysis buffer (150 mL, pH 8.0, 50 mM Tris, 20 mM NaCl, 10 mM imidazole, 50 μg/mL DNAse) was added to the cell pellet at 0° C. and homogenized by passing through an 18G needle. The cells were sonicated on ice (12×(10 seconds on, 30 seconds off), ½″ tip, 70% amplitude), and the lysate was centrifuged at 15,000×g for 30 minutes at 4° C. The clarified lysate was loaded onto a Ni-NTA column (GE HisTrap™ FF Crude, 5 mL), washed with wash buffer (36 mL, pH 8.0, 50 mM Tris, 20 mM NaCl, 17.6 mM imidazole), and eluted directly onto an ion-exchange column (GE HiTrap® Q FF, 1 mL) with elution buffer (32 mL, pH 8.0, 50 mM Tris, 20 mM NaCl, 105 mM imidazole). The column was washed with wash buffer (10 mL, pH 7.0, 50 mM Tris, 20 mM NaCl) and eluted with a gradient elution buffer (48 mL, pH 7.0, 50 mM Tris, 20 mM-1 M NaCl). Fractions containing protein were concentrated with a 10 kDa molecular weight cutoff filter (Amicon®) to a volume of ˜300 μL. The concentrated solution was loaded onto a second ion-exchange column (GE HiTrap® Q FF, 1 mL), washed with wash buffer (10 mL, pH 5.0, 20 mM NaOAc), and eluted with a gradient elution buffer (40 mL, pH 5.0, 20→608 mM NaOAc). Fractions with protein were concentrated with a 10 kDa molecular weight cutoff filter (Amicon®) to a volume of ˜200 μL. The protein was then purified by size exclusion column chromatography (BioRad ENrich™ SEC 70 10×300 column, 25 mL) on an FPLC with elution buffer (pH 7.0, 50 mM NaH₂PO₄, 20 mM NaCl). Pure fractions were collected and concentrated with a 10 kDa molecular weight cutoff filter (Amicon®) to a volume of −1 mL. The protein concentration was determined by A₂₈₀ measurements in denaturing buffer (pH 7.0, 6 M guanidinium, 30 mM MOPS) on a spectrophotometer. The protein solution was stored at 4° C.

### Example 63: Protein Labeling Experiment: In-Gel Fluorescence Analysis

Buffer A: pH 7.0, 50 mM NaH₂PO₄, 20 mM NaCl

### Time-Dependent Labeling

A stock solution of HaloTag® protein (15.3 μL, 2.01 mg/mL in buffer A) was added to 29.7 μL of buffer A to prepare a HaloTag® working solution (solution A, 19.7 μM, 45 μL). Solution A was aliquoted as follows:
Reaction A: 5 μL of solution A (blank)
Reaction B: 5 μL of solution A (control for the absence of alkyne 21′)
Reaction C: 5 μL of solution A (control for the absence of hydroxylamine 22′)
Reaction D: 25 μL of solution A

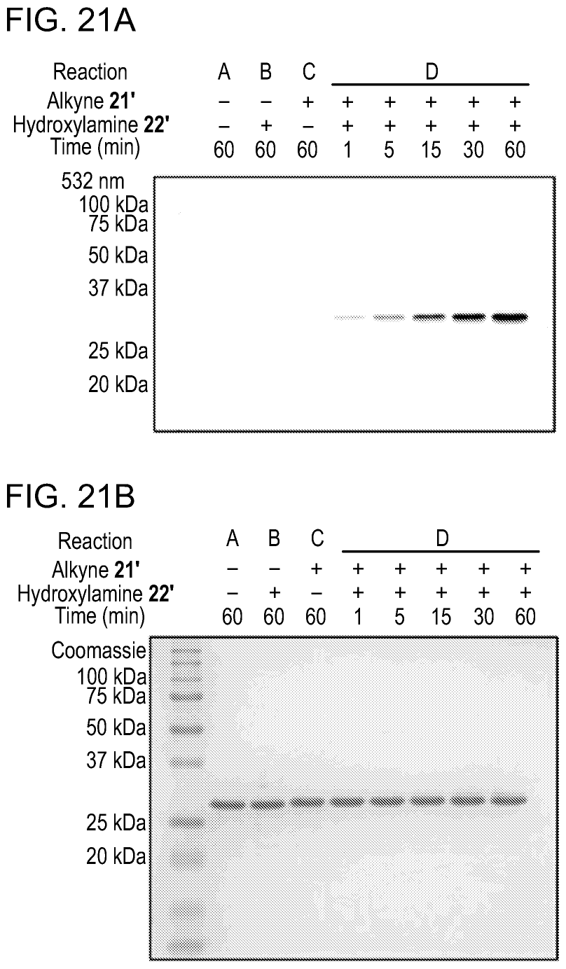

A solution of alkyne 21′ in DMSO (700 μM) was added to reactions C (0.22 μL) and D (1.10 μL). DMSO (0.22 μL) was added to reactions A and B as a vehicle control. The reaction mixtures were incubated at room temperature for 10 minutes. Then, an aqueous solution of hydroxylamine 22′ (5 mM) was added to reactions B (0.22 μL) and D (1.10 μL) at a final concentration of 200 μM. Deionized water (0.22 μL) was added to reactions A and C as a vehicle control. The reaction mixtures were incubated at room temperature in the dark. At each time point (1, 5, 15, 30, and 60 minutes), an aliquot of reaction D was removed (5.44 μL), quenched with 5×SDS sample loading buffer (1.36 μL), flash frozen in liquid nitrogen, and stored at −20° C. All remaining samples were eventually quenched with 5×SDS sample loading buffer (1.36 μL) and flash frozen after incubation for 60 minutes. Each solution (6.2 μL) was loaded onto a 12-well 12% SDS-PAGE gel. The gel was run at 0° C. and at 100 V for 2 hours. In-gel fluorescence was imaged with a Typhoon™ FLA 9500 (GE) at 532 nm with a photomultiplier tube (PMT) setting of 500 V. The experiment was carried out in triplicate (FIG. 21A-FIG. 21B).

### Concentration-Dependent Labeling

A stock solution of HaloTag® protein (15.3 μL, 2.01 mg/mL in buffer A) was added to 29.7 μL of buffer A to prepare a HaloTag® working solution (solution A, 19.7 μM, 45 μL). Solution A was aliquoted as follows:
Reaction A: 5 μL of solution A (blank)
Reaction B: 5 μL of solution A (control for the absence of alkyne 21′)
Reaction C: 5 μL of solution A (control for the absence of hydroxylamine 22′)
Reaction D: 20 μL of solution A

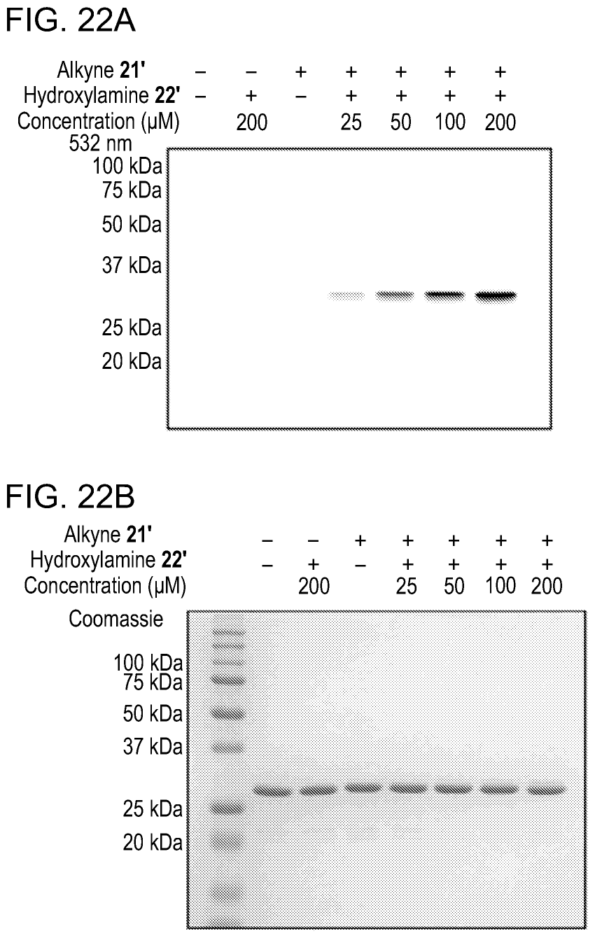

A solution of alkyne 21′ in DMSO (700 μM) was added to reactions C (0.22 μL) and D (0.88 μL). DMSO (0.22 μL) was added to reactions A and B as a vehicle control. The reaction mixtures were incubated for 10 minutes at room temperature. Reaction D was aliquoted (5.22 μL) to prepare 4 samples. Then, an aqueous solution of hydroxylamine 22′ (0.625, 1.25, 2.5, and 5 mM, 0.22 μL) was added to each reaction D aliquot at a final concentration 25, 50, 100, and 200 μM. An aqueous solution of hydroxylamine 22′ (5 mM, 0.22 μL) was also added to reaction B at a final concentration 200 μM. Deionized water (0.22 μL) was added to reactions A and C as a vehicle control. The reaction mixtures were incubated for 60 minutes at room temperature in the dark. The reaction mixtures were quenched with 5×SDS sample loading buffer (1.36 μL). Each solution (6.2 μL) was loaded onto a 12-well 12% SDS-PAGE gel. The gel was run at 0° C. and at 100 V for 2 hours. In-gel fluorescence was imaged with a Typhoon™ FLA 9500 (GE) at 532 nm with a photomultiplier tube (PMT) setting of 500 V. The experiment was carried out in triplicate (FIG. 22A-FIG. 22B).

### Example 64: Intact Mass Spectrometry Analysis

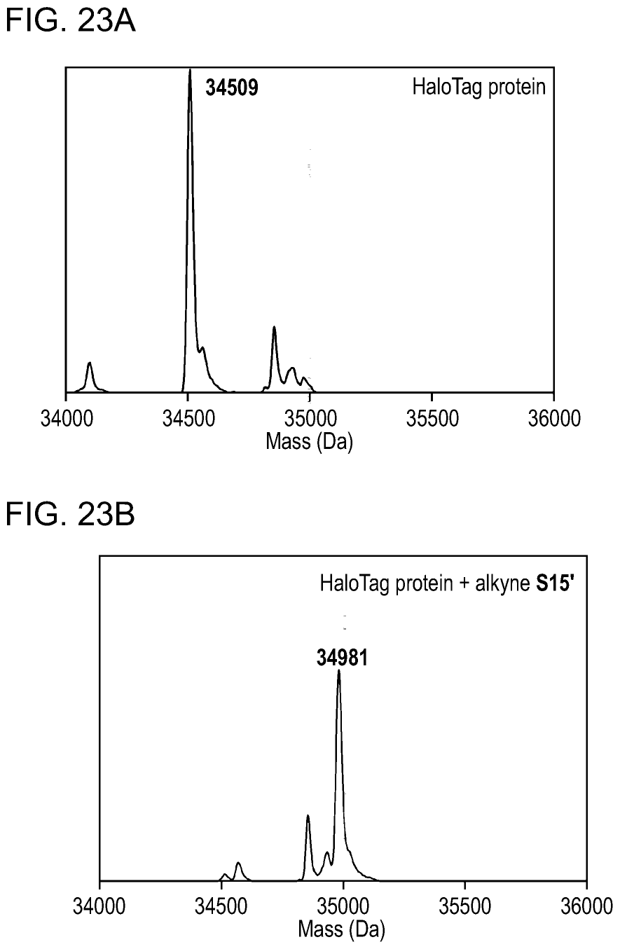

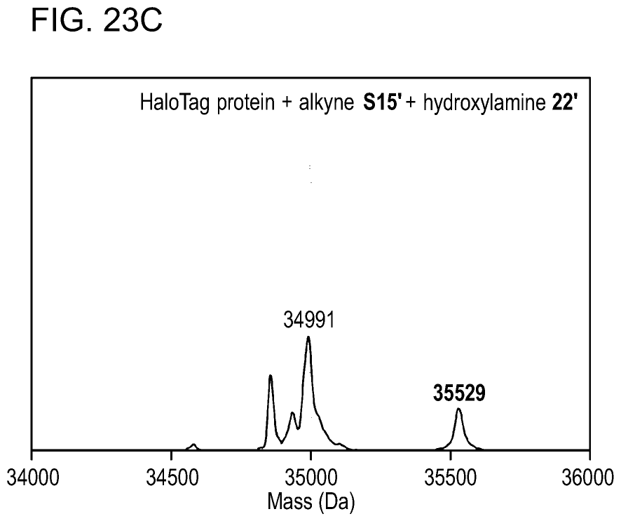

A solution of alkyne S15′ in DMSO (700 μM, 2.20 μL) was added to a solution of HaloTag® protein (19.7 μM in buffer A, 50 μL, 1 equiv; Buffer A: pH 7.0, 50 mM NaH₂PO₄, 20 mM NaCl) to make solution A. After 10 minutes, hydroxylamine 22′ (5 mM, 1.74 μL) was added to solution A (15.7 μL) at a final concentration of 500 μM. The reaction solution was incubated at room temperature for 8 hours in the dark. The sample was then snap frozen using liquid nitrogen and stored at −80° C. until further analysis. ESI-MS analysis was performed on an LTQ XL™ ion trap mass spectrometer (ThermoFisher Scientific™) (FIG. 23).

### Example 65: Live Cell Labeling Experiment

SignalSeq-HaloTag-PDGFR Gene (SEQ ID NO: 2):
ACTATAGGGCTAGCGCCACCATGGAGACAGACACACTCCTGCTATGGGTACTGCTG
CTCTGGGTTCCAGGTTCCACTGGTGACTATCCATATGATGTTCCAGATTATGCTGGAT
CCGAAATCGGTACTGGCTTTCCATTCGACCCCCATTATGTGGAAGTCCTGGGCGAGC
GCATGCACTACGTCGATGTTGGTCCGCGCGATGGCACCCCTGTGCTGTTCCTGCACG
GTAACCCGACCTCCTCCTACGTGTGGCGCAACATCATCCCGCATGTTGCACCGACCC
ATCGCTGCATTGCTCCAGACCTGATCGGTATGGGCAAATCCGACAAACCAGACCTG
GGTTATTTCTTCGACGACCACGTCCGCTTCATGGATGCCTTCATCGAAGCCCTGGGTC
TGGAAGAGGTCGTCCTGGTCATTCACGACTGGGGCTCCGCTCTGGGTTTCCACTGGG
CCAAGCGCAATCCAGAGCGCGTCAAAGGTATTGCATTTATGGAGTTCATCCGCCCTA
TCCCGACCTGGGACGAATGGCCAGAATTTGCCCGCGAGACCTTCCAGGCCTTCCGCA
CCACCGACGTCGGCCGCAAGCTGATCATCGATCAGAACGTTTTTATCGAGGGTACGC
TGCCGATGGGTGTCGTCCGCCCGCTGACTGAAGTCGAGATGGACCATTACCGCGAG
CCGTTCCTGAATCCTGTTGACCGCGAGCCACTGTGGCGCTTCCCAAACGAGCTGCCA
ATCGCCGGTGAGCCAGCGAACATCGTCGCGCTGGTCGAAGAATACATGGACTGGCT
GCACCAGTCCCCTGTCCCGAAGCTGCTGTTCTGGGGCACCCCAGGCGTTCTGATCCC
ACCGGCCGAAGCCGCTCGCCTGGCCAAAAGCCTGCCTAACTGCAAGGCTGTGGACA
TCGGCCCGGGTCTGAATCTGCTGCAAGAAGACAACCCGGACCTGATCGGCAGCGAG
ATCGCGCGCTGGCTGTCTACTCTGGAGATTTCCGGTGAAAACCTGTACTTCCAATCC
GCTGTGGGCCAGGACACGCAGGAGGTCATCGTGGTGCCACACTCCTTGCCCTTTAAG
GTGGTGGTGATCTCAGCCATCCTGGCCCTGGTGGTGCTCACCATCATCTCCCTTATCA
TCCTCATCATGCTTTGGCAGAAGAAGCCACGTGGTGGTTCTGGTATGGTTAGC.
sfGFP Gene (SEQ ID NO: 3):
ATGGTTAGCAAAGGTGAAGAACTGTTTACCGGCGTTGTGCCGATTCTGGTGGAACTG
GATGGTGATGTGAATGGCCATAAATTTAGCGTTCGTGGCGAAGGCGAAGGTGATGC
GACCAACGGTAAACTGACCCTGAAATTTATTTGCACCACCGGTAAACTGCCGGTTCC
GTGGCCGACCCTGGTGACCACCCTGACCTATGGCGTTCAGTGCTTTAGCCGCTATCC
GGATCATATGAAACGCCATGATTTCTTTAAAAGCGCGATGCCGGAAGGCTATGTGCA
GGAACGTACCATTAGCTTCAAAGATGATGGCACCTATAAAACCCGTGCGGAAGTTA
AATTTGAAGGCGATACCCTGGTGAACCGCATTGAACTGAAAGGTATTGATTTTAAAG
AAGATGGCAACATTCTGGGTCATAAACTGGAATATAATTTCAACAGCCATGCGGTGT
ATATTACCGCCGATAAACAGAAAAATGGCATCAAAGCGAACTTTAAAATCCGTCAC
AACGTGGAAGATGGTAGCGTGCAGCTGGCGGATCATTATCAGCAGAATACCCCGAT
TGGTGATGGCCCGGTGCTGCTGCCGGATAATCATTATCTGAGCACCCAGAGCGTTCT
GAGCAAAGATCCGAATGAAAAACGTGATCATATGGTGCTGCTGGAATTTGTTACCG
CCGCGGGCATTACCCACGGTATGGATGAACTGTATAAAGGCAGCTAA.

pHTC-HaloTag-sfGFP Plasmid: sfGFP gene is inserted into pHTC CMV-neo vector (Promega™)

Cloning for pHTC-Igx chain leader Seq-HaloTag-PDGFR-sfGFP: The Igx chain leader Seq-HaloTag-PDGFR was amplified from SignalSeq-HaloTag-PDGFR gene with primers SignalSeq-HaloTag-PDGFR-Gibson-F and SignalSeq-HaloTag-PDGFR-Gibson-R and gel purified. The pHTC-sfGFP vector was amplified from pHTC-HaloTag-sfGFP plasmid with primers pHTC-sfGFP-Gibson-F and pHTC-sfGFP-Gibson-R and gel purified. The vector (25 ng) was combined with the SignalSeq-HaloTag-PDGFR PCR product in a 1:2 molar ratio and assembled by Gibson Assembly® using the NEBuilder® HiFi DNA Assembly master mix (15 minutes, 50° C.). The assembly mixture (0.5 μL) was transformed into chemically competent DH5α cells and selected on ampicillin LB/agar plates. Several of the resulting colonies were used to inoculate 5 mL of LB media with 50 μg/mL ampicillin. Plasmids were isolated using a miniprep kit and sequence verified. The verified plasmid used in cell transfection were prepared with a midiprep kit.

Primers:

SignalSeq-HaloTag-PDGFR-Gibson-F:
(SEQ ID NO: 4)
5′-ACTATAGGGCTAGCGCCAC-3′
SignalSeq-HaloTag-PDGFR-Gibson-R:
(SEQ ID NO: 5)
5′-GCTAACCATACCAGAACCACC-3′
pHTC-sfGFP-Gibson-F:
(SEQ ID NO: 6)
5′-GGTGGTTCTGGTATGGTTAGCAAAGG-3′
pHTC-sfGFP-Gibson-R:
(SEQ ID NO: 7)
5′-GGTGGCGCTAGCCCTATAGTG-3′

Cell Culture: HEK-293T cells were cultured in Dulbecco's Modified Eagle Medium (DMEM, Corning®) containing 10% fetal bovine serum (FBS, Sigma), 100 units/mL penicillin, and 0.1 mg/mL streptomycin (Sigma) in a humidified chamber at 37° C. with 5% CO₂. Cells were passaged and dissociated with 0.25% trypsin, 0.1% EDTA in HBSS (Corning®). Cells tested negative for mycobacteria by the MycoAlert PLUS Mycoplasma Detection Kit (Lonza) following the manufacturer's protocol.

Preparation for 12-Well Plate: Coverslips (15 mm circle, thickness 0.13-0.17 mm) were acid-washed according to the Cold Spring Harbor protocol (Fischer et al., Cold Spring Harb. Protoc. 2008:pdb.prot4988 (2008)). A coverslip was added to each well in a 12-well plate followed by an aqueous solution of poly-D-lysine (0.1 mg/mL, 30-70 kDa, 800 μL). The plate was gently shaken to distribute the solution evenly, incubated at room temperature for 1 hour, washed using 1 mL of autoclaved deionized water for each well, and dried overnight.

Live Cell Labeling Experiment: HEK293T cells were seeded at a density of 200,000 cells per well in 1 mL of DMEM with 10% FBS (Sigma), 100 units/mL penicillin, and 0.1 mg/mL streptomycin (Sigma). Cells were then grown in a humidified chamber at 37° C. with 5% CO₂. Upon reaching ˜80% confluency, the cells in each well were transfected with Mirus Bio™ TransIT™-293 transfection reagent (3 μL) using plasmid pHTC-Igx chain leader Seq-HaloTag-PDGFR-sfGFP (1 μg) in OptiMEM I reduced serum medium (100 μL). After 36 hours, cells were washed with PBS supplemented with Mg²⁺ and Ca²⁺(3×1 mL). A solution of alkyne 21′ in serum-free DMEM (10 μM, 400 μL) was added to each well requiring alkyne. Serum-free DMEM (400 μL) was added as a vehicle control to control wells lacking alkyne. The plate was incubated at room temperature for 5 minutes in the dark. Then, a solution of hydroxylamine 22′ in serum-free DMEM (50 μM, 400 μL) was added to the appropriate wells requiring the hydroxylamine. Serum-free DMEM (400 μL) was added as a vehicle control to control wells lacking hydroxylamine. The plate was incubated at room temperature for 1 hour in the dark. After the incubation, each well was aspirated and gently washed with PBS supplemented with Mg²⁺ and Ca²⁺(1 mL). Paraformaldehyde solution (4% w/v in H₂O, 1 mL) was then added to each well and incubated at room temperature for 20 minutes to fix the cells. Each well was aspirated and gently washed with PBS supplemented with Mg²⁺ and Ca²⁺(3×1 mL). An aqueous solution of Hoechst 33342 (1 μg/mL, 500 μL) was added to the wells for nuclear staining and incubated at room temperature for 10 minutes. After gently washing the cells with PBS supplemented with Mg²⁺ and Ca²⁺(3×1 mL), each coverslip was lifted, washed by dipping twice into deionized water in a 200 mL beaker, and mounted on a microscopy slide using aqueous mounting media (20 mM Tris pH 8.0, 0.5% N-propyl gallate, 90% glycerol).

Confocal microscopy experiment: Slides were imaged at the Confocal and Light Microscopy Core at Dana-Farber Cancer Institute using a Leica SP5 laser scanning confocal microscope. Images were acquired with a HCX PL APO lambda blue 63×/1.4 oil objective at 2048×2048 px. Hoechst 33342 was imaged with a 405 nm laser and a 441.5/71 filter and false-colored blue; GFP was imaged with a 488 nm laser and a 521/30 filter and false-colored green; and TAMRA was imaged with a 561 nm laser and a 626/60 filter and false-colored red. All images presented in a single panel were imaged with the same master gain and laser power and displayed with the same contrast and brightness settings. Images were processed with Fiji ImageJ software.

### Example 66: Computational Details

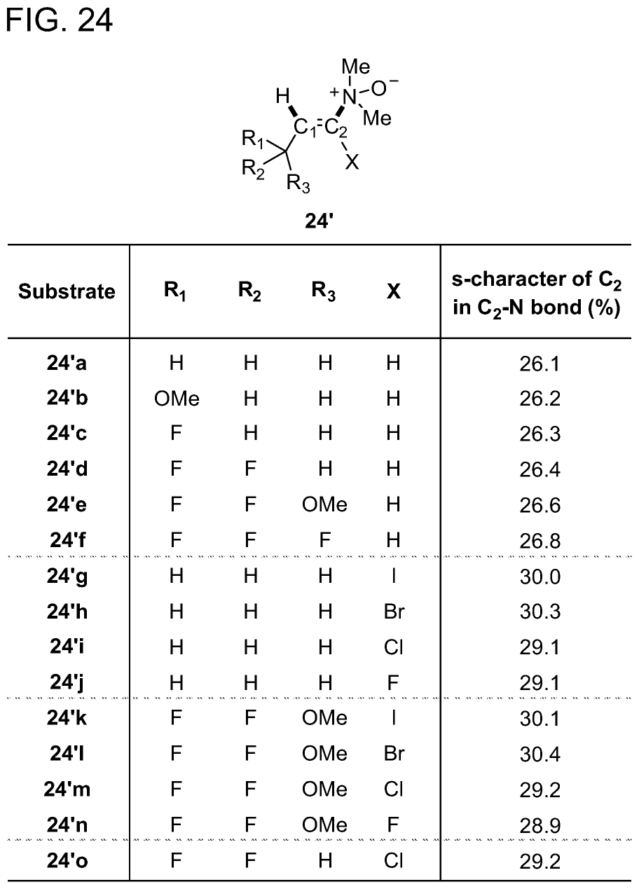

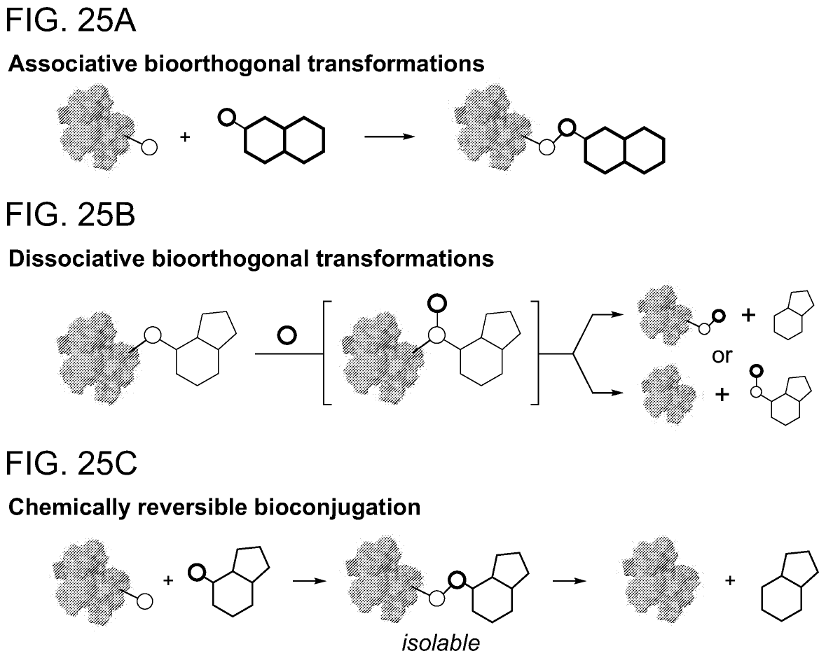

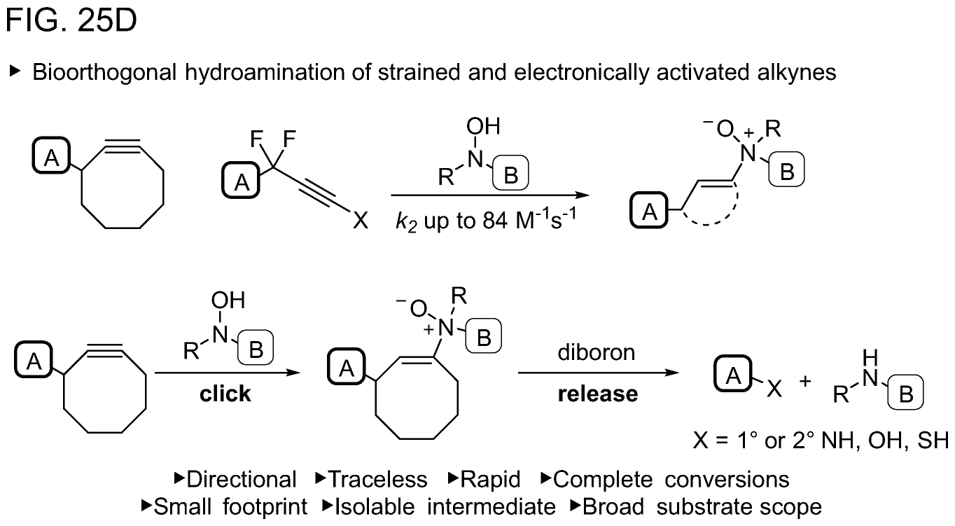

All calculations were conducted with Gaussian 09 software (Frisch et al., Gaussian 16, Revision C.01, Gaussian, Inc., Wallingford CT, 2019). Geometry optimizations for all species were performed using the M06-2X functional (Zhao et al., Theor. Chem. Acc. 120:215-241 (2008)). The LANL2DZ basis set with ECP (Wadt et al., The Journal of Chemical Physics 82:284 (1985)) was employed for Br and I, and the 6-31G(d) basis set was used for other atoms. Frequency analysis was carried out to ensure the stationary point was a minimum or a transition state, and intrinsic reaction coordinates were computed for all transition states. The single-point calculation was carried out by using M06-2X with a mixed basis set (def2qzvp (Weigend et al., Phys. Chem. Chem. Phys. 7:3297-3305 (2005)) for Br and I and 6-311G(2d,p) for the other atoms). Hybridization was analyzed using natural bond orbital (NBO) (Glendening et al., NBO Version 3.1; Reed et al., Chem. Rev. 88(6):899-926 (1988)) analysis implemented in Gaussian (FIG. 24).

Cartesian coordinates of optimized structures (1)

23′a

Lowest three frequencies(cm⁻¹): 359.3374, 360.2208, 678.1376

E(RM062X)=−116.631296046

| | | | |
|---|---|---|---|
| C | −0.21905400 | 0.00001000 | 0.00002300 |
| C | −1.42333600 | −0.00000700 | −0.00000500 |
| H | −2.48916200 | 0.00001100 | −0.00003400 |
| C | 1.24234900 | 0.00000000 | −0.00000300 |
| H | 1.62983200 | 0.99676200 | 0.22460000 |
| H | 1.62980100 | −0.69292800 | 0.75089100 |
| H | 1.62977900 | −0.30386900 | −0.97555300 |

23′a-TS

Lowest three frequencies(cm⁻¹): −323.4525, 75.2458, 94.5023

E(RM062X)=−326.907889023

| | | | |
|---|---|---|---|
| C | −1.60799700 | −0.00013500 | 0.14244300 |
| C | −0.67288500 | −0.00006200 | 0.97237800 |
| C | −3.07118900 | 0.00010000 | −0.09279700 |
| N | 0.99148700 | 0.00000400 | −0.05073200 |
| C | 1.72994700 | 1.22482600 | 0.22442800 |
| H | 1.98898200 | 1.25292900 | 1.28706000 |
| H | 2.64248600 | 1.27708800 | −0.37964700 |
| C | 1.73065000 | −1.22441000 | 0.22428400 |
| H | 1.99047900 | −1.25211100 | 1.28673500 |
| H | 2.64277700 | −1.27640100 | −0.38042600 |
| O | 0.51468000 | −0.00009600 | −1.32311900 |
| H | −0.52028900 | −0.00011900 | −1.08897000 |
| H | 1.07857800 | −2.06374800 | −0.01613100 |
| H | 1.07762600 | 2.06376500 | −0.01666500 |
| H | −0.23035500 | −0.00177000 | 1.95509400 |
| H | −3.35985100 | 0.87210000 | −0.68692700 |
| H | −3.65840000 | 0.00654200 | 0.83069500 |
| H | −3.36103500 | −0.87944400 | −0.67516400 |

24′a

Lowest three frequencies(cm⁻¹): 104.5983, 181.0617, 209.2961

E(RM062X)=−326.958575934

| | | | |
|---|---|---|---|
| C | 1.55265493 | 0.00002839 | 0.29892655 |
| C | 0.51114004 | −0.00001356 | −0.51473244 |
| C | 2.97670072 | −0.00001859 | −0.15860643 |
| N | −0.86025188 | 0.00000146 | 0.05387361 |
| C | −1.56540225 | 1.22222732 | −0.43438145 |
| H | −1.58865368 | 1.26332860 | −1.52729872 |
| H | −2.57053294 | 1.18592797 | −0.01662578 |
| C | −1.56539915 | −1.22227647 | −0.43426319 |
| H | −1.58865445 | −1.26348079 | −1.52717660 |
| H | −2.57052981 | −1.18595222 | −0.01650844 |
| O | −0.87517145 | 0.00007000 | 1.40546076 |
| H | 1.30790800 | 0.00009024 | 1.35887390 |
| H | −1.02129235 | −2.06753588 | −0.01794227 |
| H | −1.02130492 | 2.06753067 | −0.01813632 |
| H | 0.55262773 | −0.00010416 | −1.60113193 |
| H | 3.50360778 | 0.87875551 | 0.22566603 |
| H | 3.05807887 | −0.00027367 | −1.24827464 |
| H | 3.50371471 | −0.87853894 | 0.22609520 |

23′b

Lowest three frequencies(cm⁻¹): 93.8046, 179.0552, 236.2805

E(RM062X)=−231.127878821

| | | | |
|---|---|---|---|
| C | 1.42213200 | 0.11695800 | 0.00004700 |
| C | 2.57042700 | −0.24223600 | −0.00001000 |
| H | 3.58468200 | −0.57093800 | −0.00007300 |
| C | 0.03521900 | 0.58587600 | −0.00001200 |
| H | −0.13621500 | 1.21952600 | 0.88565800 |
| H | −0.13617300 | 1.21946200 | −0.88573500 |
| O | −0.84232600 | −0.51217800 | 0.00000400 |
| C | −2.18325600 | −0.09295600 | −0.00000300 |
| H | −2.41868300 | 0.50667300 | −0.89164900 |
| H | −2.80340500 | −0.98972400 | −0.00007200 |
| H | −2.41872400 | 0.50657000 | 0.89170100 |

23′b-TS

Lowest three frequencies(cm⁻¹): −354.2930, 69.8033, 98.9867

E(RM062X)=−441.413268021

| | | | |
|---|---|---|---|
| C | 0.54007600 | 0.68588300 | 0.00037300 |
| C | −0.57326200 | 1.25028900 | 0.00093200 |
| C | 2.00287100 | 0.77293300 | 0.00028000 |
| N | −1.92636300 | −0.14069500 | −0.00003900 |
| C | −2.71151700 | −0.05822700 | 1.22474800 |
| H | −3.22416600 | 0.90776700 | 1.25315900 |
| H | −3.44684400 | −0.86861600 | 1.27367500 |
| C | −2.71120000 | −0.05625700 | −1.22492200 |
| H | −3.22379400 | 0.90980800 | −1.25197200 |
| H | −3.44655400 | −0.86653200 | −1.27532800 |
| O | −1.16389000 | −1.26971900 | −0.00080700 |
| H | −0.21859700 | −0.83167500 | −0.00059900 |
| H | −2.02025400 | −0.12999000 | −2.06433700 |
| H | −2.02080400 | −0.13324000 | 2.06424400 |
| H | −1.23844300 | 2.09849400 | 0.00152600 |
| H | 2.34669600 | 1.33335600 | −0.88572400 |
| H | 2.34681700 | 1.33282100 | 0.88656900 |
| O | 2.58341400 | −0.51706400 | −0.00014100 |
| C | 3.98297100 | −0.45124500 | −0.00019600 |
| H | 4.36183600 | −1.47438000 | −0.00058400 |
| H | 4.36636600 | 0.07084900 | −0.89064500 |
| H | 4.36646200 | 0.07021300 | 0.8905830 |

24′b

Lowest three frequencies(cm⁻¹): 48.4210, 85.1016, 93.1584

E(RM062X)=−441.459145504

| | | | |
|---|---|---|---|
| C | −0.51736900 | 0.22884500 | −0.30043400 |
| C | 0.53480400 | 0.26931500 | 0.49662700 |
| C | −1.89792700 | 0.58218900 | 0.15139000 |
| N | 1.87526200 | −0.05787600 | −0.04570500 |
| C | 2.76559300 | 1.12137800 | 0.17344000 |
| H | 2.82307100 | 1.39193400 | 1.23181500 |
| H | 3.74254100 | 0.84422300 | −0.21977200 |
| C | 2.39942200 | −1.23916500 | 0.70472100 |
| H | 2.44373700 | −1.04535200 | 1.78037300 |
| H | 3.38769900 | −1.44650400 | 0.29703300 |
| O | 1.85573000 | −0.35115200 | −1.36348800 |
| H | −0.33860800 | −0.05842700 | −1.33317600 |
| H | 1.72378400 | −2.06118100 | 0.47735000 |
| H | 2.34377300 | 1.92959600 | −0.42065800 |
| H | 0.51922500 | 0.52372700 | 1.55360400 |
| H | −1.92584600 | 0.76154800 | 1.23957600 |
| H | −2.21534400 | 1.51918400 | −0.33912900 |
| O | −2.77003600 | −0.46229300 | −0.20394500 |
| C | −4.10385300 | −0.15957700 | 0.11066300 |
| H | −4.71465600 | −1.00887700 | −0.19802800 |
| H | −4.23750800 | 0.00353100 | 1.19057600 |
| H | −4.44827900 | 0.74137100 | −0.41860700 |

23′c

Lowest three frequencies(cm⁻¹): 181.5180, 223.3463, 269.2025

E(RM062X)=−255.169752458

| | | | |
|---|---|---|---|
| C | 1.02317483 | 0.01539317 | 0.11271702 |
| C | 2.20106871 | 0.12598969 | −0.10900903 |
| C | −0.41627936 | −0.09093325 | 0.38885905 |
| F | −0.91657532 | −1.23692017 | −0.20520780 |
| H | 3.24469790 | 0.21955313 | −0.30753847 |
| C | −1.18989506 | 1.10411492 | −0.14168998 |
| H | −0.83925456 | 2.02444594 | 0.32919266 |
| H | −1.04691335 | 1.18218642 | −1.22088374 |
| H | −2.25268118 | 0.97301536 | 0.07039284 |
| H | −0.56508558 | −0.19430641 | 1.47044460 |

23′c-TS

Lowest three frequencies(cm⁻¹): −218.5957, 42.4795, 71.4697

E(RM062X)=−465.457353742

| | | | |
|---|---|---|---|
| C | 0.78615988 | 0.11617977 | −0.40298223 |
| C | −0.19646667 | 0.78729298 | −0.77404053 |
| C | 2.23282620 | −0.12344932 | −0.40008988 |
| F | 2.54555253 | −1.25053261 | 0.36192225 |
| N | −1.78603379 | −0.00602209 | 0.05927278 |
| C | −2.40041289 | 0.95808210 | 0.96227668 |
| H | −2.73578785 | 1.82298230 | 0.38259405 |
| H | −3.25192138 | 0.51445531 | 1.48975278 |
| C | −2.67776367 | −0.51957640 | −0.97213125 |
| H | −3.01672008 | 0.31359906 | −1.59466297 |
| H | −3.54273681 | −1.02394391 | −0.52762409 |
| O | −1.23728656 | −1.03489055 | 0.76673933 |
| H | −0.24142003 | −0.89990628 | 0.51958233 |
| H | −2.11002242 | −1.22473573 | −1.57883402 |
| H | −1.64160817 | 1.26862155 | 1.68029530 |
| H | −0.70252247 | 1.57424866 | −1.30779302 |
| C | 3.01413969 | 1.04548216 | 0.18138395 |
| H | 2.87498826 | 1.93753248 | −0.43195069 |
| H | 2.66029999 | 1.25428431 | 1.19304015 |
| H | 4.07635822 | 0.79533767 | 0.22422440 |
| H | 2.57075382 | −0.34047066 | −1.42124893 |

24′c

Lowest three frequencies(cm⁻¹): 38.6587, 97.4610, 132.5120

E(RM062X)=−465.501893812

| | | | |
|---|---|---|---|
| C | −0.73225805 | −0.09729830 | −0.18717441 |
| C | 0.34547459 | −0.01241759 | 0.57088933 |
| C | −2.12357322 | −0.07778442 | 0.36469569 |
| F | −2.80089930 | −1.19122807 | −0.11427276 |
| N | 1.68364472 | 0.01231620 | −0.06608503 |
| C | 2.37008233 | 1.27448253 | 0.34375001 |
| H | 2.45084362 | 1.35420655 | 1.43166881 |
| H | 3.35153790 | 1.25505720 | −0.12756299 |
| C | 2.45146957 | −1.17046577 | 0.42998744 |
| H | 2.53265045 | −1.16866111 | 1.52075466 |
| H | 3.43148902 | −1.11798430 | −0.04177666 |
| O | 1.62642141 | −0.03675281 | −1.41361108 |
| H | −0.57277408 | −0.14957208 | −1.26229493 |
| H | 1.91418745 | −2.04680027 | 0.07326574 |
| H | 1.77697590 | 2.08692838 | −0.07143196 |
| H | 0.35336997 | 0.03832872 | 1.65711305 |
| C | −2.89083568 | 1.15777344 | −0.07097371 |
| H | −2.41678321 | 2.06043092 | 0.32091415 |
| H | −2.90840251 | 1.21710210 | −1.16192101 |
| H | −3.91873733 | 1.10571047 | 0.29430479 |
| H | −2.10530505 | −0.15162419 | 1.45985883 |

23′d

Lowest three frequencies(cm⁻¹): 178.3136, 185.5486, 256.0556

E(RM062X)=−354.419951628

| | | | |
|---|---|---|---|
| C | 1.15360611 | 0.02719644 | 0.00013653 |
| C | 2.35093939 | 0.13416524 | 0.00026999 |
| C | −0.31933908 | −0.04857065 | −0.00002840 |
| F | −0.72603731 | −0.75368907 | −1.09135357 |
| F | −0.72629950 | −0.75399254 | 1.09099629 |
| H | 3.41464267 | 0.21693102 | 0.00013323 |
| C | −0.99512129 | 1.30199456 | 0.00007999 |
| H | −0.70482138 | 1.85961674 | 0.89095245 |
| H | −0.70430302 | 1.86005671 | −0.89035444 |
| H | −2.07499774 | 1.14381640 | −0.00026435 |

23′d-TS

Lowest three frequencies(cm⁻¹): −189.4483, 30.9094, 61.2334

E(RM062X)=−564.709736873

| | | | |
|---|---|---|---|
| C | 0.54870157 | −0.06028759 | −0.27217244 |
| C | −0.39465570 | −0.03520563 | −1.07605472 |
| C | 1.99418931 | −0.04681651 | −0.00436292 |
| F | 2.25909627 | −0.63510785 | 1.20442248 |
| F | 2.66960968 | −0.80089058 | −0.92572030 |
| N | −2.09134424 | 0.01387983 | 0.02972068 |
| C | −2.82084136 | 1.24867655 | −0.21893712 |
| H | −3.14342500 | 1.26537094 | −1.26391664 |
| H | −3.69519859 | 1.33093320 | 0.43682847 |
| C | −2.86382434 | −1.20097910 | −0.18918434 |
| H | −3.18514570 | −1.23302749 | −1.23420248 |
| H | −3.74114907 | −1.23483678 | 0.46681391 |
| O | −1.57151628 | 0.02068401 | 1.29402223 |
| H | −0.57223908 | −0.01898116 | 1.07650022 |
| H | −2.21431742 | −2.05098200 | 0.02043037 |
| H | −2.14211087 | 2.08101880 | −0.03174142 |
| H | −0.89032104 | −0.03757582 | −2.03004142 |
| C | 2.57980564 | 1.34846898 | 0.00480230 |
| H | 2.44714909 | 1.81058602 | −0.97373852 |
| H | 2.08559515 | 1.95457283 | 0.76473492 |
| H | 3.64409826 | 1.27113630 | 0.23524596 |

24′d

Lowest three frequencies(cm⁻¹): 11.0771, 63.1956, 75.0997

E(RM062X)=−564.758917799

| | | | |
|---|---|---|---|
| C | 0.48136634 | 0.04446264 | 0.42864490 |
| C | −0.51570688 | −0.07550130 | −0.42602678 |
| C | 1.91456936 | −0.04096888 | 0.00375842 |
| F | 2.51484299 | −1.05611486 | 0.69295583 |
| F | 1.98694106 | −0.37974749 | −1.32029802 |
| N | −1.90308638 | 0.03527021 | 0.07804335 |
| C | −2.57781135 | 1.14160135 | −0.66656038 |
| H | −2.56201583 | 0.96797071 | −1.74615923 |
| H | −3.59662658 | 1.18767793 | −0.28524200 |
| C | −2.59494103 | −1.26174821 | −0.19594322 |
| H | −2.57793794 | −1.50899947 | −1.26101655 |
| H | −3.61376777 | −1.14576863 | 0.17036984 |
| O | −1.96419119 | 0.29556635 | 1.40114125 |
| H | 0.23066268 | 0.22529888 | 1.47031359 |
| H | −2.07148352 | −2.01133914 | 0.39400750 |
| H | −2.04315198 | 2.05182964 | −0.40222238 |
| H | −0.41149047 | −0.25939802 | −1.49036210 |
| C | 2.70438543 | 1.22725827 | 0.22438341 |
| H | 2.27268661 | 2.03924521 | −0.36238559 |
| H | 2.68452158 | 1.49707038 | 1.28129225 |
| H | 3.73650981 | 1.05712822 | −0.08748715 |

23′e

Lowest three frequencies(cm⁻¹): 58.1385, 158.2871, 161.5802

E(RM062X)=−429.632123202

| | | | |
|---|---|---|---|
| C | −1.48961400 | −0.24496500 | −0.00008900 |
| C | −2.64408400 | −0.57388700 | −0.00003300 |
| C | −0.08045900 | 0.17523000 | −0.00001700 |
| O | 0.72286100 | −0.91172300 | −0.00046000 |
| C | 2.11967800 | −0.60846100 | −0.00004200 |
| H | 2.38813600 | −0.04438300 | 0.89560800 |
| H | 2.62845600 | −1.57024600 | −0.00136400 |
| H | 2.38815900 | −0.04194100 | −0.89414200 |
| F | 0.16924400 | 0.96344700 | −1.07864700 |
| F | 0.16939600 | 0.96267500 | 1.07913700 |
| H | −3.66852500 | −0.87225100 | 0.00024500 |

23′e-TS

Lowest three frequencies(cm⁻¹): −185.3547, 18.0896, 55.7181

E(RM062X)=−639.922859192

| | | | |
|---|---|---|---|
| C | 0.17234200 | −0.32063800 | 0.26525700 |
| C | −0.81677500 | −0.68319000 | 0.91201300 |
| C | 1.62185200 | −0.30700100 | 0.04276000 |
| O | 2.07698700 | 0.97348400 | 0.03209900 |
| C | 3.49025500 | 1.09126300 | −0.11472800 |
| H | 3.82896200 | 0.56907300 | −1.01285100 |
| H | 3.69115400 | 2.15701200 | −0.21006900 |
| H | 4.00420500 | 0.69121700 | 0.76199800 |
| F | 2.30190500 | −1.03201200 | 0.98018100 |
| F | 1.91624000 | −0.92584200 | −1.13748800 |
| N | −2.44593600 | 0.12421100 | −0.03257900 |
| C | −3.29639700 | −0.93086800 | −0.56364300 |
| H | −3.69993700 | −1.51367300 | 0.26934400 |
| H | −4.12106700 | −0.51561400 | −1.15460400 |
| C | −3.12357500 | 1.06527100 | 0.84704200 |
| H | −3.52227100 | 0.52135600 | 1.70821600 |
| H | −3.94096200 | 1.57709800 | 0.32582000 |
| O | −1.83411100 | 0.79443700 | −1.05591300 |
| H | −0.85763500 | 0.57538800 | −0.85965300 |
| H | −2.38845900 | 1.79653200 | 1.18387800 |
| H | −2.67966100 | −1.57180200 | −1.19408500 |
| H | −1.38529800 | −1.16776800 | 1.68413100 |

24′e

Lowest three frequencies(cm⁻¹): 35.1100, 59.5559, 86.8622

E(RM062X)=−639.973929364

| | | | |
|---|---|---|---|
| C | 0.10901958 | −0.02415733 | −0.44138775 |
| C | −0.88800788 | −0.09598628 | 0.41771754 |
| C | 1.52722521 | −0.21820483 | −0.01580967 |
| O | 2.26071373 | 0.87294151 | −0.33951856 |
| C | 3.65221055 | 0.76273992 | −0.04126150 |
| H | 4.11037919 | −0.03029490 | −0.63602195 |
| H | 4.08966579 | 1.72422922 | −0.30337327 |
| H | 3.80221278 | 0.56393322 | 1.02251672 |
| F | 1.61253790 | −0.46944753 | 1.32478409 |
| F | 2.04384419 | −1.33047297 | −0.61047222 |
| N | −2.26762077 | 0.09979448 | −0.07764772 |
| C | −3.04670422 | −1.13779290 | 0.23480682 |
| H | −3.03876167 | −1.35862136 | 1.30580229 |
| H | −4.05754833 | −0.95855198 | −0.12789060 |
| C | −2.85268505 | 1.27039221 | 0.64603972 |
| H | −2.83885443 | 1.12161865 | 1.72935475 |
| H | −3.86917540 | 1.38033452 | 0.27165797 |
| O | −2.32259165 | 0.33095799 | −1.40560899 |
| H | −0.11666295 | 0.17921604 | −1.48292369 |
| H | −2.25549960 | 2.13224993 | 0.35505408 |
| H | −2.58352805 | −1.93740419 | −0.33982110 |
| H | −0.78764649 | −0.28912666 | 1.48076140 |

23′f

Lowest three frequencies(cm⁻¹): 183.8674, 184.4368, 464.8412

E(RM062X)=−414.359346599

| | | | |
|---|---|---|---|
| C | 1.12939945 | −0.00075114 | −0.00016555 |
| C | 2.32966176 | −0.00030874 | −0.00007923 |
| C | −0.33983472 | −0.00006294 | 0.00000813 |
| F | −0.81828106 | 1.09633152 | −0.59336705 |
| F | −0.81982523 | −1.06176147 | −0.65236873 |
| H | 3.39711944 | −0.00003197 | 0.00013313 |
| F | −0.81883576 | −0.03381795 | 1.24587875 |

23′f-TS

Lowest three frequencies(cm⁻¹): −177.6883, 20.6223, 57.0280

E(RM062X)=−624.652385677

| | | | |
|---|---|---|---|
| C | −0.58633100 | −0.00563300 | 0.32269700 |
| C | 0.35343300 | −0.00058800 | 1.12105200 |
| C | −2.01529100 | −0.00095800 | 0.00219400 |
| F | −2.37178600 | 1.15846300 | −0.56973100 |
| F | −2.32661400 | −0.98519200 | −0.85488800 |
| N | 2.09492600 | −0.00087400 | −0.02146600 |
| C | 2.84881800 | 1.22611100 | 0.19030900 |
| H | 3.19621900 | 1.25375100 | 1.22695400 |
| H | 3.70945900 | 1.28517600 | −0.48630600 |
| C | 2.85317500 | −1.22342800 | 0.20007200 |
| H | 3.19936500 | −1.24220800 | 1.23733200 |
| H | 3.71491900 | −1.28426700 | −0.47498200 |
| O | 1.55986400 | −0.00679200 | −1.28345800 |
| H | 0.57150000 | −0.00716200 | −1.06975500 |
| H | 2.18816000 | −2.06875300 | 0.02099400 |
| H | 2.18042600 | 2.06763200 | 0.00592000 |
| H | 0.87845200 | 0.00258300 | 2.05743800 |
| F | −2.80212400 | −0.16430500 | 1.07821800 |

24′f

Lowest three frequencies(cm⁻¹): 39.4176, 98.2763, 142.2168

E(RM062X)=−624.702123419

| | | | |
|---|---|---|---|
| C | −0.49092715 | 0.00013286 | −0.44219660 |
| C | 0.49649247 | −0.00015038 | 0.43142259 |
| C | −1.91743417 | 0.00000541 | −0.00961916 |
| F | −2.56273221 | 1.07839973 | −0.47325834 |
| F | −2.56282384 | −1.07786842 | −0.47436192 |
| N | 1.88475293 | 0.00001199 | −0.07580626 |
| C | 2.56375780 | 1.22623658 | 0.44754458 |
| H | 2.54396124 | 1.26146592 | 1.54023795 |
| H | 3.58355699 | 1.19092565 | 0.06806258 |
| C | 2.56382900 | −1.22628185 | 0.44724171 |
| H | 2.54406584 | −1.26177634 | 1.53992886 |
| H | 3.58362249 | −1.19084130 | 0.06775437 |
| O | 1.94936863 | 0.00018459 | −1.42280479 |
| H | −0.24522229 | 0.00043143 | −1.49966413 |
| H | 2.03609067 | −2.07207373 | 0.01101553 |
| H | 2.03596086 | 2.07208008 | 0.01149578 |
| H | 0.38244381 | −0.00049315 | 1.51077505 |
| F | −2.04633360 | −0.00063519 | 1.32729997 |

23′g

Lowest three frequencies(cm⁻¹): 155.6285, 156.2428, 382.7313

E(RM062X)=−413.661714997

| | | | |
|---|---|---|---|
| C | 2.20887300 | 0.00005300 | 0.00005000 |
| C | 1.00276600 | 0.00027800 | −0.00003100 |
| I | −1.00853800 | −0.00002000 | 0.00000000 |
| C | 3.66918800 | −0.00009500 | 0.00000000 |
| H | 4.05593900 | 0.77190100 | 0.66973600 |
| H | 4.05574000 | −0.96621400 | 0.33362700 |
| H | 4.05585400 | 0.19394000 | −1.00348200 |

23′g-TS

Lowest three frequencies(cm⁻¹): −244.9092, 62.7725, 69.2259

E(RM062X)=−623.944112471

| | | | |
|---|---|---|---|
| C | −1.10927000 | 1.70863600 | 0.00014900 |
| C | −0.38798200 | 0.70337100 | 0.00009000 |
| N | −1.56390400 | −0.95278600 | −0.00003200 |
| C | −1.39314100 | −1.71952800 | −1.22731300 |
| H | −0.39032300 | −2.15127600 | −1.24179600 |
| H | −2.14305000 | −2.51587000 | −1.28935400 |
| C | −1.39344600 | −1.71885500 | 1.22775600 |
| H | −0.39069200 | −2.15075900 | 1.24269800 |
| H | −2.14347900 | −2.51505100 | 1.29014300 |
| O | −2.78002700 | −0.33477300 | −0.00041300 |
| H | −2.46268400 | 0.64497300 | −0.00028800 |
| H | −1.51234400 | −1.03244700 | 2.06645700 |
| H | −1.51199800 | −1.03363600 | −2.06644600 |
| I | 1.55695800 | −0.06107500 | −0.00001300 |
| C | −1.42073000 | 3.14990100 | −0.00007500 |
| H | −2.01377700 | 3.41517800 | −0.87981300 |
| H | −2.01821200 | 3.41489000 | 0.87673200 |
| H | −0.51727100 | 3.76753400 | 0.00225600 |

24′g

Lowest three frequencies(cm⁻¹): 65.0037, 90.6528, 175.8901

E(RM062X)=−623.990813043

| | | | |
|---|---|---|---|
| C | −0.65633631 | 1.87051942 | −0.00023924 |
| C | −0.52448726 | 0.55338517 | −0.00021773 |
| N | −1.76323276 | −0.30028617 | 0.00000874 |
| C | −1.79135717 | −1.15168718 | −1.22943599 |
| H | −0.94743344 | −1.84202026 | −1.26730582 |
| H | −2.74135695 | −1.68159194 | −1.19404169 |
| C | −1.79136170 | −1.15140081 | 1.22964789 |
| H | −0.94769957 | −1.84205631 | 1.26747243 |
| H | −2.74158675 | −1.68091822 | 1.19456184 |
| O | −2.87089422 | 0.47257417 | −0.00010772 |
| H | −1.70237948 | 2.17266180 | 0.00033664 |
| H | −1.78107438 | −0.46238181 | 2.07193578 |
| H | −1.78151778 | −0.46278149 | −2.07181718 |
| I | 1.35863289 | −0.44596647 | 0.00000560 |
| C | 0.41515629 | 2.90777100 | −0.00002302 |
| H | 0.30383325 | 3.55547044 | −0.87488853 |
| H | 0.31198762 | 3.54642990 | 0.88254241 |
| H | 1.41978433 | 2.48329503 | −0.00668368 |

23′h

Lowest three frequencies(cm⁻¹): 157.8453, 158.5314, 336.5966

E(RM062X)=−2690.27976725

| | | | |
|---|---|---|---|
| C | 1.77726300 | 0.00010200 | −0.00079200 |
| C | 0.57300700 | 0.00104300 | −0.00027000 |
| Br | −1.26873400 | −0.00011100 | 0.00006800 |
| C | 3.23848500 | −0.00029600 | 0.00032600 |
| H | 3.62527800 | 0.69038100 | −0.75281500 |
| H | 3.62365800 | 0.30678800 | 0.97563300 |
| H | 3.62421100 | −0.99838400 | −0.22077700 |

23′h-TS

Lowest three frequencies(cm⁻¹): −234.0393, 58.3141, 65.5530

E(RM062X)=−2900.56401868

| | | | |
|---|---|---|---|
| C | −0.62475700 | 1.74717700 | 0.00004500 |
| C | 0.02847300 | 0.70282800 | 0.00001200 |
| N | −1.20464900 | −0.91775000 | 0.00000900 |
| C | −1.06509400 | −1.69197700 | −1.22702100 |
| H | −0.07705300 | −2.15567500 | −1.24271400 |
| H | −1.84213100 | −2.46230300 | −1.28710000 |
| C | −1.06501000 | −1.69220600 | 1.22688900 |
| H | −0.07704900 | −2.15608700 | 1.24235800 |
| H | −1.84217400 | −2.46240400 | 1.28695800 |
| O | −2.40217400 | −0.25614400 | 0.00009600 |
| H | −2.05797500 | 0.70359200 | 0.00007700 |
| H | −1.16207500 | −1.00429700 | 2.06722700 |
| H | −1.16241100 | −1.00394100 | −2.06722600 |
| Br | 1.74614300 | −0.14771600 | −0.00000100 |
| C | −0.95426300 | 3.18280200 | 0.00001400 |
| H | −1.55151900 | 3.44153300 | 0.87877100 |
| H | −0.05758900 | 3.80973100 | 0.00019100 |
| H | −1.55116900 | 3.44154500 | −0.87897800 |

24′h

Lowest three frequencies(cm⁻¹): 67.5984, 100.2293, 175.9725

E(RM062X)=−2900.61404253

| | | | |
|---|---|---|---|
| C | −0.27803928 | 1.77322943 | −0.00028109 |
| C | 0.04812420 | 0.49229022 | −0.00023433 |
| N | 1.48108386 | 0.07095462 | −0.00007628 |
| C | 1.77179652 | −0.73008605 | 1.23050655 |
| H | 1.17798770 | −1.64415686 | 1.26978339 |
| H | 2.83842625 | −0.94292433 | 1.19232303 |
| C | 1.77179116 | −0.73119136 | −1.22999487 |
| H | 1.17825284 | −1.64551851 | −1.26829461 |
| H | 2.83850798 | −0.94347864 | −1.19173163 |
| O | 2.28483549 | 1.15726166 | −0.00036553 |
| H | 0.59847548 | 2.41732799 | −0.00019087 |
| H | 1.55385671 | −0.07682608 | −2.07173115 |
| H | 1.55435506 | −0.07468224 | 2.07156315 |
| Br | −1.26818130 | −0.95534128 | 0.00005382 |
| C | −1.64884710 | 2.36119400 | −0.00001086 |
| H | −1.77992898 | 2.99811752 | −0.87987841 |
| H | −2.43027957 | 1.60090981 | 0.00171417 |
| H | −1.77853180 | 3.00078313 | 0.87810493 |

23′i

Lowest three frequencies(cm⁻¹): 192.9577, 193.2793, 370.0489

E(RM062X)=−576.222606833

| | | | |
|---|---|---|---|
| C | 1.09037400 | −0.00001500 | −0.00009000 |
| C | −0.11343400 | −0.00026800 | −0.00014000 |
| Cl | −1.76384800 | 0.00006000 | 0.00004000 |
| C | 2.55156800 | 0.00007000 | 0.00006200 |
| H | 2.93795900 | 0.12518800 | 1.01447700 |
| H | 2.93826400 | −0.94093100 | −0.39873200 |
| H | 2.93814900 | 0.81600900 | −0.61541100 |

23′i-TS

Lowest three frequencies(cm⁻¹): −253.4726, 45.4070, 67.0878

E(RM062X)=−786.508236040

| | | | |
|---|---|---|---|
| C | 1.62516297 | −0.42528770 | −0.00006317 |
| C | 0.70607279 | 0.40318170 | 0.00020530 |
| Cl | 0.16431329 | 2.02102369 | 0.00002805 |
| N | −1.05556982 | −0.57687371 | 0.00002540 |
| C | −1.80269541 | −0.31910273 | 1.22640891 |
| H | −2.11024913 | 0.72811199 | 1.24307179 |
| H | −2.68296908 | −0.96890286 | 1.28365388 |
| C | −1.80207481 | −0.31913949 | −1.22677127 |
| H | −2.10955487 | 0.72808673 | −1.24371113 |
| H | −2.68237460 | −0.96887525 | −1.28439194 |
| O | −0.58885611 | −1.86012498 | 0.00014650 |
| H | 0.42177844 | −1.66176792 | 0.00026081 |
| H | −1.13882053 | −0.52353193 | −2.06762019 |
| H | −1.13986215 | −0.52332697 | 2.06763634 |
| C | 3.03058832 | −0.87609137 | 0.00002762 |
| H | 3.23588717 | −1.49347433 | 0.87908363 |
| H | 3.23515077 | −1.49703262 | −0.87668526 |
| H | 3.73520257 | −0.03893625 | −0.00196899 |

24′i

Lowest three frequencies(cm⁻¹): 72.4928, 116.5398, 179.3935

E(RM062X)=−786.560658839

| | | | |
|---|---|---|---|
| C | −1.23436690 | −1.06433711 | 0.00001335 |
| C | −0.37444988 | −0.05850611 | −0.00003220 |
| Cl | −0.82132077 | 1.62699979 | 0.00000747 |
| N | 1.09531682 | −0.33108555 | 0.00001261 |
| C | 1.71147220 | 0.25988926 | −1.22977206 |
| H | 1.57966946 | 1.34202581 | −1.27166748 |
| H | 2.76330034 | −0.01674284 | −1.18980187 |
| C | 1.71161261 | 0.25996816 | 1.22968599 |
| H | 1.58024789 | 1.34216520 | 1.27128840 |
| H | 2.76330570 | −0.01716929 | 1.18978885 |
| O | 1.33438517 | −1.66050484 | 0.00007527 |
| H | −0.74254442 | −2.03356940 | −0.00027212 |
| H | 1.23066759 | −0.23523608 | 2.07099951 |
| H | 1.23074469 | −0.23578067 | −2.07092938 |
| C | −2.72266433 | −0.95668127 | 0.00004656 |
| H | −3.13438093 | −1.46144322 | −0.87905420 |
| H | −3.13441478 | −1.46214951 | 0.87872918 |
| H | −3.06606379 | 0.07854364 | 0.00045189 |

23′j

Lowest three frequencies(cm⁻¹): 241.6304, 242.2611, 428.5901

E(RM062X)=−215.846441310

| | | | |
|---|---|---|---|
| C | 0.50821100 | 0.00003600 | −0.00000800 |
| C | −0.69038800 | −0.00006200 | 0.00000500 |
| C | 1.97130000 | −0.00000100 | −0.00000300 |
| H | 2.35861700 | −0.91182300 | −0.46064000 |
| H | 2.35868000 | 0.85485100 | −0.55929400 |
| H | 2.35863300 | 0.05695400 | 1.01997300 |
| F | −1.97896300 | 0.00002100 | 0.00000000 |

23′j-TS

Lowest three frequencies(cm⁻¹): −228.7575, 36.9601, 64.4327

E(RM062X)=−426.141643225

| | | | |
|---|---|---|---|
| C | 1.68403900 | 0.01122900 | −0.00003100 |
| C | 0.69612200 | 0.73900300 | 0.00012700 |
| N | −1.01199400 | −0.30319200 | −0.00002900 |
| C | −1.77152600 | −0.06987800 | 1.22326900 |
| H | −2.09198400 | 0.97343000 | 1.24272300 |
| H | −2.64360000 | −0.73192200 | 1.27579100 |
| C | −1.77223300 | −0.06841800 | −1.22260300 |
| H | −2.09373000 | 0.97459700 | −1.24005300 |
| H | −2.64366800 | −0.73123500 | −1.27591600 |
| O | −0.51508200 | −1.58560700 | −0.00096800 |
| H | 0.47316300 | −1.36997300 | −0.00082100 |
| H | −1.11180600 | −0.26423300 | −2.06778100 |
| H | −1.11091500 | −0.26785200 | 2.06779300 |
| C | 3.08195700 | −0.45823700 | −0.00000800 |
| H | 3.28307600 | −1.07945900 | 0.87727500 |
| H | 3.28437200 | −1.07460300 | −0.88041500 |
| H | 3.79550800 | 0.37124900 | 0.00276800 |
| F | 0.06156100 | 1.89833400 | 0.00022900 |

24′j

Lowest three frequencies(cm⁻¹): 84.9082, 156.7781, 179.7476

E(RM062X)=−426.201123752

| | | | |
|---|---|---|---|
| C | 1.47072700 | −0.64788200 | −0.00009700 |
| C | 0.45628900 | 0.19253600 | 0.00003500 |
| N | −0.95319400 | −0.22670900 | 0.00001600 |
| C | −1.60916500 | 0.32562700 | 1.22789200 |
| H | −1.52302400 | 1.41279400 | 1.27091300 |
| H | −2.64739600 | 0.00181400 | 1.18360400 |
| C | −1.60890600 | 0.32526500 | −1.22812600 |
| H | −1.52261500 | 1.41241300 | −1.27141200 |
| H | −2.64717800 | 0.00156200 | −1.18400800 |
| O | −1.06538200 | −1.57325500 | 0.00023200 |
| H | 1.17763500 | −1.69112600 | 0.00015800 |
| H | −1.10850700 | −0.15272600 | −2.06798200 |
| H | −1.10893200 | −0.15220700 | 2.06793300 |
| C | 2.90207000 | −0.21449400 | 0.00008200 |
| H | 3.42143800 | −0.60650700 | 0.87921900 |
| H | 3.42085700 | −0.60329300 | −0.88084500 |
| H | 2.98989100 | 0.87325500 | 0.00195100 |
| F | 0.56412800 | 1.53230200 | −0.00002400 |

23′k

Lowest three frequencies(cm⁻¹): 63.1933, 70.5718, 93.1439

E(RM062X)=−7349.28733923

| | | | |
|---|---|---|---|
| C | −0.81224500 | −0.14889700 | 0.00012000 |
| C | 0.38846400 | −0.07010600 | 0.00010300 |
| C | −2.27804400 | −0.26014200 | 0.00012000 |
| O | −2.83130300 | 0.97303900 | −0.00032800 |
| C | −4.26096600 | 0.97421400 | −0.00003900 |
| H | −4.64339700 | 0.48016100 | 0.89553400 |
| H | −4.55309200 | 2.02231700 | −0.00118400 |
| H | −4.64377200 | 0.47812200 | −0.89432400 |
| F | −2.68917800 | −0.97727000 | −1.07829300 |
| F | −2.68925200 | −0.97651400 | 1.07900100 |
| I | 2.39006200 | 0.07261800 | −0.00010600 |

23′k-TS

Lowest three frequencies(cm⁻¹): −142.9341, 17.8359, 46.2952

E(RM062X)=−7559.58157091

| | | | |
|---|---|---|---|
| C | 1.02765800 | 0.01702900 | −0.09010000 |
| C | −0.19851200 | −0.03329600 | −0.03141800 |
| C | 2.42813900 | −0.40540900 | −0.16983400 |
| O | 3.21177500 | 0.42544200 | 0.56750000 |
| C | 4.59023000 | 0.05698200 | 0.60169300 |
| H | 4.99115600 | −0.02405100 | −0.41134200 |
| H | 5.09777600 | 0.85711300 | 1.13739700 |
| H | 4.72135000 | −0.88940800 | 1.13043100 |
| F | 2.59387700 | −1.69201400 | 0.24650900 |
| F | 2.83288600 | −0.40621600 | −1.47103600 |
| N | −0.90561600 | 1.96072700 | −0.01082800 |
| C | −1.67294500 | 2.26665300 | −1.20986400 |
| H | −2.60912800 | 1.70466400 | −1.18499200 |
| H | −1.88685600 | 3.34002200 | −1.27183300 |
| C | −1.57778200 | 2.27974600 | 1.24092100 |
| H | −2.51388400 | 1.71979900 | 1.29481700 |
| H | −1.78450500 | 3.35419700 | 1.30817400 |
| O | 0.30185800 | 2.60746800 | −0.06061100 |
| H | 0.94107800 | 1.83018000 | −0.07317000 |
| H | −0.92244300 | 1.98053700 | 2.05984200 |
| H | −1.08260700 | 1.95966200 | −2.07404200 |
| I | −1.94610000 | −1.13259000 | 0.05807100 |

24′k

Lowest three frequencies(cm⁻¹): 34.9384, 55.4428, 66.6192

E(RM062X)=−937.001378035

| | | | |
|---|---|---|---|
| C | −0.49641762 | 1.35617970 | 0.06769505 |
| C | 0.57723945 | 0.58943824 | 0.00250124 |
| C | −1.91103358 | 0.87227013 | 0.19351599 |
| O | −2.27044294 | 0.16132510 | −0.90081019 |
| C | −3.58443119 | −0.39382615 | −0.83943845 |
| H | −4.32169665 | 0.38611667 | −0.63554344 |
| H | −3.76824722 | −0.83013350 | −1.81945119 |
| H | −3.63547565 | −1.16715380 | −0.06988494 |
| F | −2.08210241 | 0.13381444 | 1.32484701 |
| F | −2.72963428 | 1.94718271 | 0.35415435 |
| N | 1.90949057 | 1.28003636 | −0.12632632 |
| C | 2.76846559 | 0.93296214 | 1.04987349 |
| H | 2.98418224 | −0.13517052 | 1.09686481 |
| H | 3.67580297 | 1.52080958 | 0.92579187 |
| C | 2.56575257 | 0.86147737 | −1.40541593 |
| H | 2.77702959 | −0.20838141 | −1.42448184 |
| H | 3.47769878 | 1.45210979 | −1.46626507 |
| O | 1.74872067 | 2.61752598 | −0.15187964 |
| H | −0.30505854 | 2.42469625 | 0.02113450 |
| H | 1.88287327 | 1.14879447 | −2.20273538 |
| H | 2.22491620 | 1.27063659 | 1.93029506 |
| I | 0.55855657 | −1.53006359 | 0.06465430 |

23′1

Lowest three frequencies(cm⁻¹): 62.6283, 76.1155, 92.5372

E(RM062X)=−3002.72421290

| | | | |
|---|---|---|---|
| C | −0.25247500 | −0.12619800 | 0.00000500 |
| C | 0.94577100 | −0.03955900 | −0.00000500 |
| C | −1.71762000 | −0.25761300 | 0.00001800 |
| O | −2.28643000 | 0.96761800 | −0.00043800 |
| C | −3.71639000 | 0.95066900 | −0.00008500 |
| H | −4.09206200 | 0.45190600 | 0.89568600 |
| H | −4.02135100 | 1.99503600 | −0.00138500 |
| H | −4.09249200 | 0.44957700 | −0.89437500 |
| F | −2.11704800 | −0.98036800 | −1.07850900 |
| F | −2.11712200 | −0.97959100 | 1.07903300 |
| Br | 2.77283300 | 0.10966800 | −0.00002100 |

23′1-TS

Lowest three frequencies(cm⁻¹): −148.3298, 19.3857, 44.9796

E(RM062X)=−3213.02014902

| | | | |
|---|---|---|---|
| C | 0.72624600 | −0.21410700 | 0.05377800 |
| C | −0.49012000 | −0.33919300 | 0.00629500 |
| C | 2.17940900 | −0.37535400 | 0.11660300 |
| O | 2.79801300 | 0.72433000 | −0.39443800 |
| C | 4.22309700 | 0.64092400 | −0.42223100 |
| H | 4.54732000 | −0.14004900 | −1.11308200 |
| H | 4.57013600 | 1.61281200 | −0.76822700 |
| H | 4.61484100 | 0.44055200 | 0.57760700 |
| F | 2.56458000 | −0.57398900 | 1.40732500 |
| F | 2.59023300 | −1.48879000 | −0.54979000 |
| N | −1.40614500 | 1.54528800 | 0.02107000 |
| C | −2.13441700 | 1.80464300 | −1.21434500 |
| H | −2.99981900 | 1.14078600 | −1.26027000 |
| H | −2.46276000 | 2.84947200 | −1.25895000 |
| C | −2.18479700 | 1.75067600 | 1.23554800 |
| H | −3.04909100 | 1.08388400 | 1.21721200 |
| H | −2.51835700 | 2.79204700 | 1.31175900 |
| O | −0.28300600 | 2.33832700 | 0.06147700 |
| H | 0.44758000 | 1.65930600 | 0.05368100 |
| H | −1.54952900 | 1.50586400 | 2.08760400 |
| H | −1.46524400 | 1.59502600 | −2.04971200 |
| Br | −2.02062700 | −1.45424300 | −0.07580400 |

24′1

Lowest three frequencies(cm⁻¹): 36.6408, 57.5368, 67.7356

E(RM062X)=−3213.62376294

| | | | |
|---|---|---|---|
| C | 0.34377770 | 1.12585910 | 0.05706589 |
| C | −0.67746153 | 0.29207411 | 0.00949437 |
| C | 1.78169408 | 0.71851036 | 0.19275920 |
| O | 2.18720423 | 0.04037046 | −0.90580373 |
| C | 3.53634677 | −0.42428723 | −0.84751335 |
| H | 3.64561780 | −1.17976857 | −0.06657480 |
| H | 3.74104712 | −0.86308107 | −1.82219569 |
| H | 4.22081377 | 0.40679967 | −0.66267774 |
| F | 2.53774421 | 1.83576005 | 0.37187829 |
| F | 1.98109723 | −0.02150317 | 1.31709686 |
| N | −2.05899799 | 0.84392445 | −0.13441776 |
| C | −2.66883866 | 0.33087642 | −1.40333253 |
| H | −2.76676246 | −0.75520805 | −1.39646560 |
| H | −3.63629256 | 0.82378443 | −1.47519714 |
| C | −2.88222755 | 0.44171838 | 1.05094643 |
| H | −2.98386751 | −0.64142506 | 1.12567327 |
| H | −3.84467578 | 0.92946292 | 0.90995108 |
| O | −2.01812941 | 2.19024581 | −0.19162675 |
| H | 0.08815266 | 2.17816025 | −0.01475623 |
| H | −2.37912740 | 0.85896977 | 1.92116728 |
| H | −2.01857878 | 0.67021437 | −2.20737881 |
| Br | −0.52215021 | −1.64048939 | 0.11004360 |

23′m

Lowest three frequencies(cm⁻¹): 64.0556, 83.0180, 98.3108

E(RM062X)=−889.223270082

| | | | |
|---|---|---|---|
| C | 0.50936600 | −0.07950100 | −0.00000800 |
| C | 1.70452000 | 0.03915700 | −0.00003300 |
| C | −0.95074100 | −0.24997400 | 0.00001600 |
| O | −1.55325700 | 0.95980000 | −0.00044600 |
| C | −2.98208500 | 0.90398500 | −0.00010400 |
| H | −3.34433100 | 0.39514800 | 0.89555800 |
| H | −3.31552500 | 1.93964600 | −0.00132200 |
| H | −3.34468700 | 0.39295200 | −0.89437200 |
| F | −1.33191800 | −0.98323200 | −1.07838200 |
| F | −1.33199600 | −0.98243700 | 1.07892200 |
| Cl | 3.33643900 | 0.21193300 | −0.00002300 |

23′m-TS

Lowest three frequencies(cm⁻¹): −158.7631, 12.3495, 45.7192

E(RM062X)=−1099.51974357

| | | | |
|---|---|---|---|
| C | −0.43324200 | 0.29725100 | −0.05525400 |
| C | 0.70489800 | 0.74613400 | 0.03948900 |
| C | −1.89254500 | 0.22698300 | −0.15605400 |
| O | −2.35885100 | −0.85100500 | 0.52866400 |
| C | −3.78134000 | −0.96657300 | 0.54545800 |
| H | −4.17898600 | −0.96234300 | −0.47209300 |
| H | −3.99558400 | −1.92137600 | 1.02216700 |
| H | −4.22564800 | −0.15238200 | 1.12172600 |
| F | −2.48880900 | 1.36270300 | 0.30377100 |
| F | −2.25826000 | 0.14945400 | −1.46662100 |
| Cl | 1.81935800 | 2.00085600 | 0.20179500 |
| N | 2.03466000 | −0.87987700 | −0.01939200 |
| C | 2.86837500 | −0.84312100 | −1.21456200 |
| H | 3.54770300 | 0.00904400 | −1.14620100 |
| H | 3.44403400 | −1.77040100 | −1.31704100 |
| C | 2.77730500 | −1.01698300 | 1.22753200 |
| H | 3.45917500 | −0.17028800 | 1.32899500 |
| H | 3.34463500 | −1.95485900 | 1.24156400 |
| O | 1.12924600 | −1.90701900 | −0.12610400 |
| H | 0.26019400 | −1.40457400 | −0.11927100 |
| H | 2.05964100 | −1.01253300 | 2.04878100 |
| H | 2.21288000 | −0.72306500 | −2.07789700 |

24′m

Lowest three frequencies(cm⁻¹): 26.5794, 57.4169, 60.9363

E(RM062X)=−1099.56967962

| | | | |
|---|---|---|---|
| C | 0.21379788 | −0.83991969 | 0.01480283 |
| C | −0.75508834 | 0.05733543 | 0.03896218 |
| C | 1.66524284 | −0.50150017 | 0.17689413 |
| O | 2.10234610 | 0.22257970 | −0.88109252 |
| C | 3.47888042 | 0.59670989 | −0.81171227 |
| H | 4.11372410 | −0.28947779 | −0.74292340 |
| H | 3.68772963 | 1.13152725 | −1.73626205 |
| H | 3.65478384 | 1.24979686 | 0.04570160 |
| F | 1.88616537 | 0.17249245 | 1.33992494 |
| F | 2.37983873 | −1.65239680 | 0.29790398 |
| Cl | −0.53511118 | 1.75822446 | 0.26619200 |
| N | −2.16480865 | −0.40473098 | −0.14461594 |
| C | −2.96255163 | −0.04717017 | 1.07248971 |
| H | −2.99114107 | 1.03046902 | 1.23837049 |
| H | −3.95570045 | −0.45479033 | 0.89465116 |
| C | −2.73964905 | 0.24910496 | −1.36449632 |
| H | −2.76315136 | 1.33555714 | −1.27099936 |
| H | −3.73868684 | −0.16904526 | −1.47005087 |
| O | −2.20879528 | −1.74160298 | −0.31114502 |
| H | −0.09633499 | −1.86707935 | −0.14155804 |
| H | −2.11515593 | −0.06964387 | −2.19696467 |
| H | −2.49175256 | −0.57004867 | 1.90288112 |

23′n

Lowest three frequencies(cm⁻¹): 68.7266, 96.3219, 110.8543

E(RM062X)=−528.848717355

| | | | |
|---|---|---|---|
| C | −0.99504805 | 0.01095008 | 0.00000737 |
| C | −2.17753932 | −0.16589477 | −0.00002607 |
| C | 0.45491618 | 0.23846193 | 0.00002339 |
| O | 1.10666518 | −0.94656876 | −0.00045197 |
| C | 2.53150828 | −0.83196461 | −0.00012135 |
| H | 2.87294466 | −0.30864656 | 0.89544405 |
| H | 2.90782699 | −1.85292444 | −0.00135577 |
| H | 2.87319459 | −0.30641317 | −0.89428269 |
| F | 0.80990404 | 0.98607394 | −1.07804855 |
| F | 0.81002929 | 0.98526412 | 1.07861039 |
| F | −3.44107892 | −0.35675823 | −0.00006072 |

23′n-TS

Lowest three frequencies(cm⁻¹): −149.4606, 15.7767, 43.8617

E(RM062X)=−739.154654274

| | | | |
|---|---|---|---|
| C | 0.32665391 | 0.63876298 | 0.00823511 |
| C | −0.83151300 | 1.02016352 | 0.02950457 |
| C | 1.75685458 | 0.35779243 | −0.01595984 |
| O | 1.98113231 | −0.98746245 | 0.06379906 |
| C | 3.35836836 | −1.36068927 | 0.05991031 |
| H | 3.87055957 | −0.94523152 | 0.93050795 |
| H | 3.37225266 | −2.44830494 | 0.10459655 |
| H | 3.84553043 | −1.01964208 | −0.85632937 |
| F | 2.32548640 | 0.85101428 | −1.15108774 |
| F | 2.40010741 | 0.98899747 | 1.00527762 |
| N | −2.17446768 | −0.47541462 | −0.00671651 |
| C | −2.96903551 | −0.50118211 | 1.21809727 |
| H | −3.57219572 | 0.40739973 | 1.26201441 |
| H | −3.61713180 | −1.38445922 | 1.24351159 |
| C | −2.97732501 | −0.43260135 | −1.22560808 |
| H | −3.58051131 | 0.47698762 | −1.21431566 |
| H | −3.62582954 | −1.31312099 | −1.29613990 |
| O | −1.36454311 | −1.59528722 | −0.04094317 |
| H | −0.45755205 | −1.19644494 | −0.03083845 |
| H | −2.29506195 | −0.41254109 | −2.07600110 |
| H | −2.28110716 | −0.52869315 | 2.06370585 |
| F | −1.76497303 | 1.94004081 | 0.06674129 |

24′n

Lowest three frequencies(cm⁻¹): 11.0771, 63.1956, 75.0997

E(RM062X)=−739.211327539

| | | | |
|---|---|---|---|
| C | −0.16473087 | −0.70080097 | 0.00003922 |
| C | 0.77927339 | 0.21806194 | −0.00002233 |
| C | −1.63522043 | −0.45135100 | −0.00000652 |
| O | −1.93898200 | 0.86603424 | 0.00026896 |
| C | −3.33842117 | 1.14707909 | 0.00012814 |
| H | −3.81131061 | 0.73517523 | 0.89420426 |
| H | −3.42311035 | 2.23207114 | 0.00055919 |
| H | −3.81098509 | 0.73589964 | −0.89445121 |
| F | −2.19444099 | −1.07243378 | −1.07778255 |
| F | −2.19458379 | −1.07290591 | 1.07741471 |
| N | 2.20718725 | −0.15412404 | 0.00004716 |
| C | 2.83573122 | 0.42704719 | −1.23106066 |
| H | 2.71033862 | 1.51021643 | −1.27049379 |
| H | 3.88469025 | 0.14030704 | −1.18933467 |
| C | 2.83572655 | 0.42729075 | 1.23102274 |
| H | 2.71004353 | 1.51042757 | 1.27041591 |
| H | 3.88476866 | 0.14086313 | 1.18920506 |
| O | 2.37401446 | −1.49179250 | 0.00018569 |
| H | 0.19474781 | −1.72281723 | 0.00009474 |
| H | 2.35178229 | −0.06711011 | 2.07123173 |
| H | 2.35155328 | −0.06723744 | −2.07120446 |
| F | 0.62822023 | 1.53791490 | −0.00016524 |

23′o

Lowest three frequencies(cm⁻¹): 100.9680, 101.7185, 252.4116

E(RM062X)=−814.011105378

| | | | |
|---|---|---|---|
| C | −0.11214000 | −0.05046900 | 0.00002200 |
| C | −1.31394400 | −0.00648000 | 0.00003000 |
| C | 1.36280400 | −0.04451800 | −0.00000300 |
| F | 1.80695600 | −0.72652400 | 1.09082900 |
| F | 1.80693100 | −0.72650800 | −1.09085100 |
| Cl | −2.95600500 | 0.03698000 | −0.00000700 |
| C | 1.96231500 | 1.34158400 | 0.00000200 |
| H | 1.64182200 | 1.88246900 | −0.89085700 |
| H | 1.64180900 | 1.88247500 | 0.89085200 |
| H | 3.04925900 | 1.24298900 | 0.00000800 |

23′o-TS

Lowest three frequencies(cm⁻¹): −159.2235, 13.2230, 49.9152

E(RM062X)=−1024.30712998

| | | | |
|---|---|---|---|
| C | −0.77310600 | 0.10704500 | −0.05300700 |
| C | 0.30884300 | 0.69130200 | −0.02731800 |
| C | −2.22248700 | −0.12671100 | −0.04366100 |
| F | −2.82111100 | 0.59767300 | −1.03348900 |
| F | −2.48957900 | −1.43954300 | −0.33956500 |
| Cl | 1.25592700 | 2.09200900 | −0.02194000 |
| N | 1.80795100 | −0.74410000 | 0.02399500 |
| C | 2.62081700 | −0.70606900 | −1.18654900 |
| H | 3.19273900 | 0.22381100 | −1.20071700 |
| H | 3.30216000 | −1.56367400 | −1.22272900 |
| C | 2.57273900 | −0.68196200 | 1.26375000 |
| H | 3.14310300 | 0.24880700 | 1.28226600 |
| H | 3.25320700 | −1.53739500 | 1.34374600 |
| O | 1.03625500 | −1.87904900 | 0.02092100 |
| H | 0.11148200 | −1.48515300 | −0.02711800 |
| H | 2.71004353 | 1.51042757 | 1.27041591 |
| H | 1.94714200 | −0.73757600 | −2.04356400 |
| C | −2.87493200 | 0.20768400 | 1.27886800 |
| H | −2.43074800 | −0.39190100 | 2.07410700 |
| H | −2.73728600 | 1.26638300 | 1.49952600 |
| H | −3.94005500 | −0.01891100 | 1.20227000 |

24′o

Lowest three frequencies(cm⁻¹): 48.6881, 62.0755, 122.8034

E(RM062X)=−1024.35499954

| | | | |
|---|---|---|---|
| C | −0.57463215 | −0.72760824 | −0.00919078 |
| C | 0.46700107 | 0.08507537 | −0.01106991 |
| C | −2.01660832 | −0.30259883 | −0.03183878 |
| F | −2.21211656 | 0.66889053 | −0.96792076 |
| F | −2.75447586 | −1.37692295 | −0.43424528 |
| Cl | 0.39271908 | 1.81729605 | −0.05933101 |
| N | 1.84171927 | −0.50560291 | 0.03171678 |
| C | 2.57560046 | −0.12320439 | −1.21757785 |
| H | 2.68033451 | 0.95848909 | −1.31241678 |
| H | 3.54265625 | −0.61732910 | −1.14746344 |
| C | 2.56324173 | 0.00056476 | 1.24321826 |
| H | 2.68144008 | 1.08457267 | 1.22529972 |
| H | 3.52409887 | −0.51001160 | 1.23866441 |
| O | 1.77991368 | −1.85029170 | 0.10142407 |
| H | −0.32487289 | −1.78328718 | 0.04068768 |
| H | 1.97659277 | −0.32662827 | 2.09969894 |
| H | 2.00292192 | −0.54573469 | −2.04093235 |
| C | −2.54278014 | 0.17981897 | 1.29963147 |
| H | −2.43661465 | −0.60820337 | 2.04661378 |
| H | −1.98733121 | 1.06255076 | 1.62048420 |
| H | −3.59839845 | 0.43310871 | 1.18504094 |

25′ (NMe₂OH)

Lowest three frequencies(cm⁻¹): 251.1972, 290.6079, 315.6114

E(RM062X)=−210.302452064

| | | | |
|---|---|---|---|
| N | 0.00000000 | 0.02490000 | −0.41697400 |
| C | −1.19858000 | −0.64238700 | 0.06738400 |
| H | −1.22931400 | −1.65128600 | −0.35177800 |
| H | −1.22122900 | −0.70365200 | 1.16601500 |
| C | 1.19858300 | −0.64238200 | 0.06738400 |
| H | 1.22932400 | −1.65127900 | −0.35178200 |
| H | 1.22123100 | −0.70365100 | 1.16601500 |
| O | −0.00000300 | 1.31390800 | 0.19839700 |
| H | −0.00000300 | 1.91418700 | −0.55581600 |
| H | 2.07548200 | −0.09062800 | −0.27480500 |
| H | −2.07548200 | −0.09063900 | −0.27480900 |

### Example 67: Bioorthogonal Click and Release

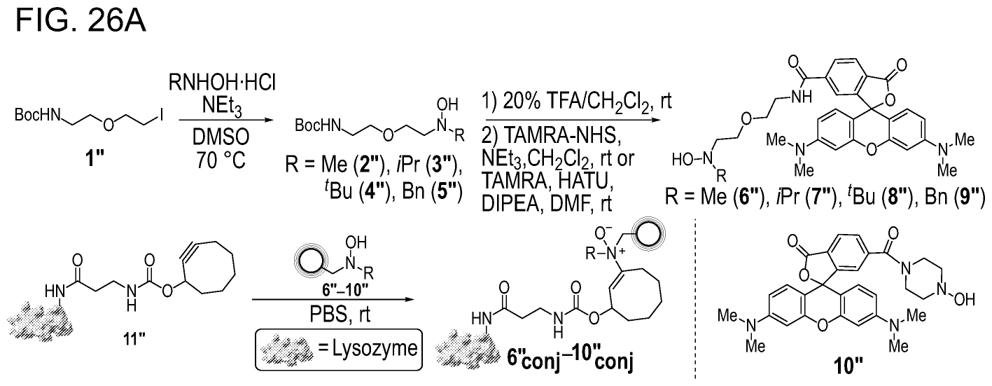

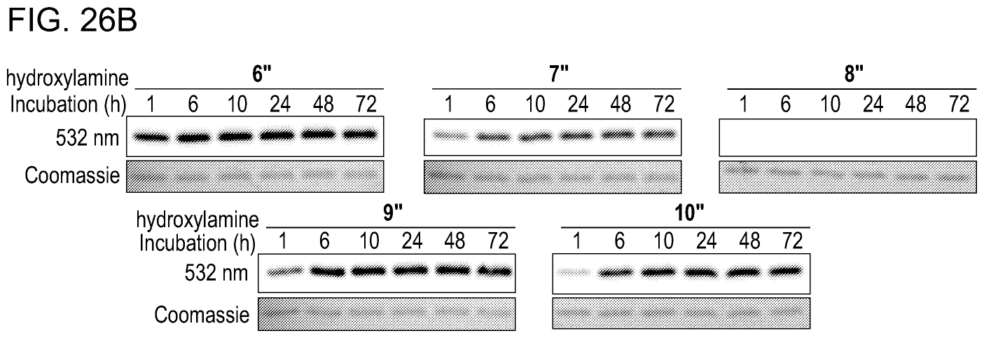

A series of N,N-dialkylhydroxylamine probes were synthesized by nucleophilic displacement of alkyl iodide 1″ with N-methyl, benzyl, isopropyl, and tert-butyl hydroxylamine hydrochloride and triethylamine in DMSO at 70° C. Trifluoroacetic acid-mediated Boc deprotection and HATU coupling of 6-carboxytetramethylrhodamine (TAMRA) or coupling of TAMRA-NHS ester to the resulting amine produced TAMRA-hydroxylamine conjugates 6″-9″ (FIG. 26A). A cyclic N-hydroxypiperazinyl-TAMRA conjugate 10″ was also prepared. Separately, lysozyme-cyclooctyne conjugate (Lys-COT) 11″ was produced by acylation of lysine residues with cyclooctyne-modified 3-aminopropanoic acid via the corresponding N-hydroxysuccinimide (NHS) ester.

The relative rates of hydroamination between TAMRA-hydroxylamines 6″-10″ (200 M) and Lys-COT 11″ (10 μM) were evaluated by monitoring the extent of lysozyme labeling over 1-72 h in phosphate-buffered saline (PBS) at room temperature by in-gel fluorescence (FIG. 26B). N-Methylhydroxylamine 6″ displayed the fastest reaction rate, nearly reaching complete conversion in 1 h. The benzyl and piperazinyl variants 9″ and 10″ demonstrated a marginally slower, yet still rapid, retro-Cope elimination reaction, reaching completion within 6 and 10 h, respectively. For all three of these substrates, the labeling proved durable over 72 h, indicating the general stability of the resulting enamine N-oxides. The more sterically encumbered hydroxylamines 7″ and 8″ featuring α-branching exhibited poor labeling.

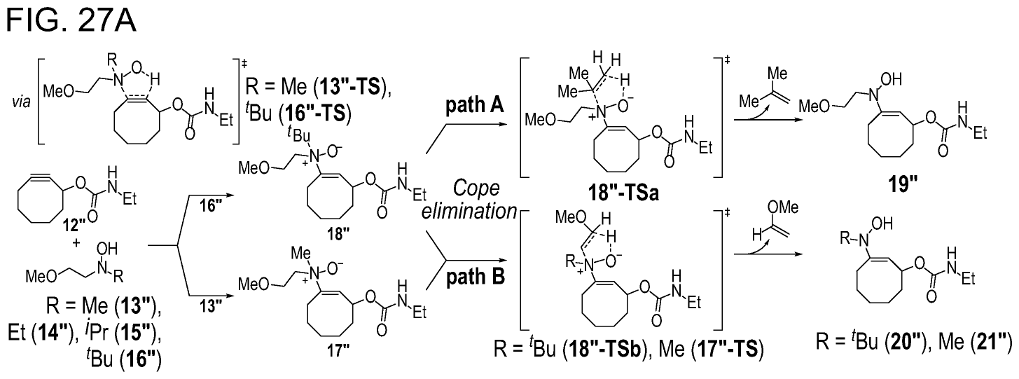

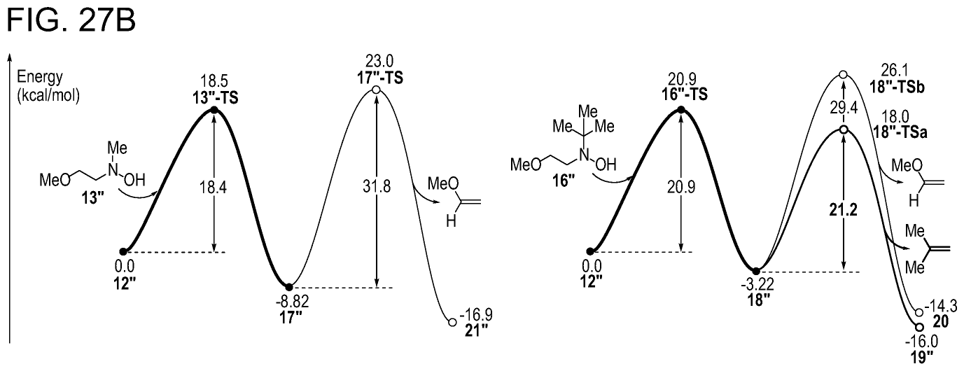

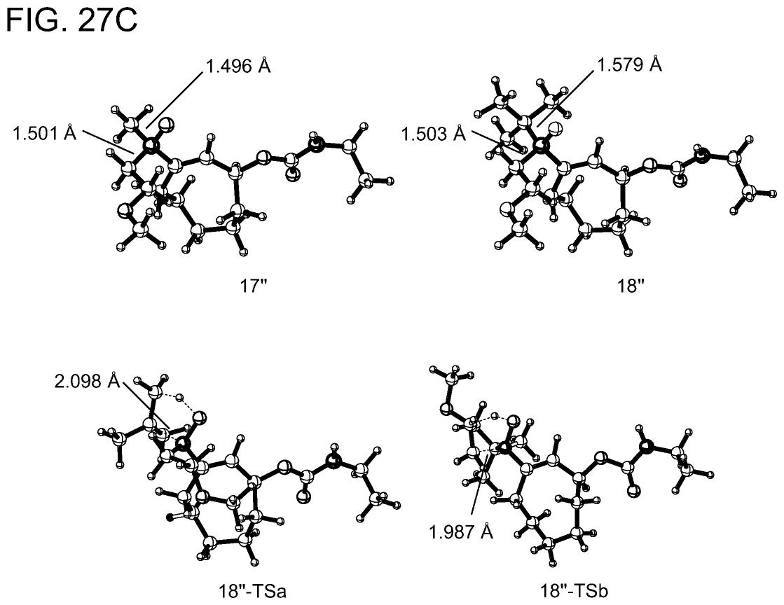

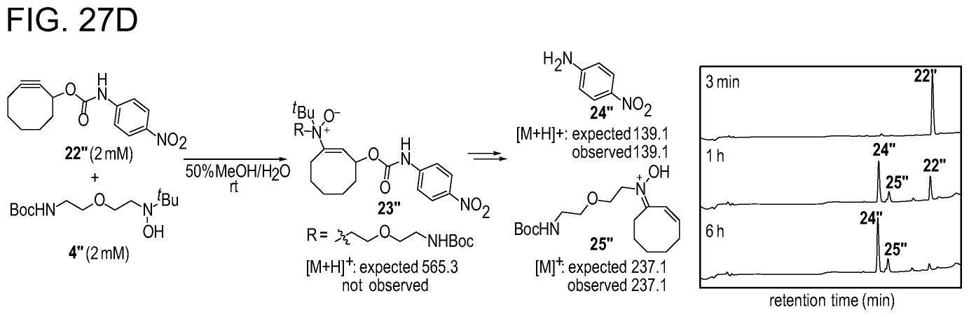

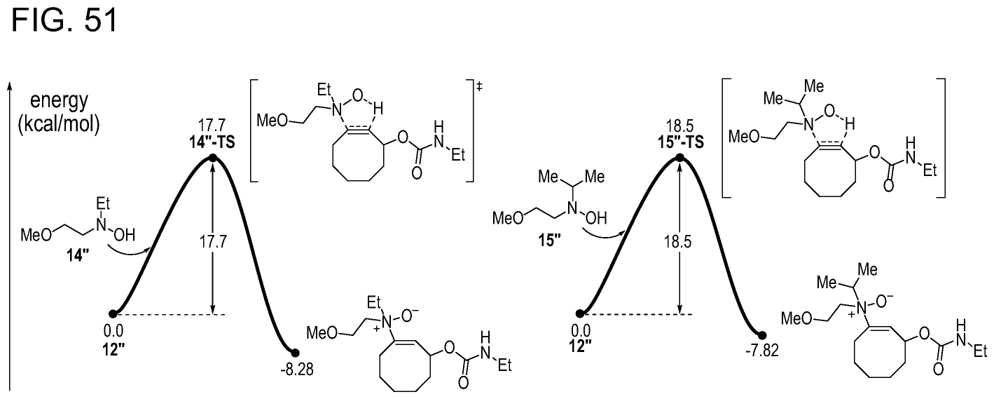

To determine whether the poor labeling was the result of retarded hydroamination or enamine N-oxide product instability, these processes were explored computationally. Density functional theory (DFT) calculations performed at the M06-2X/6-31G(d,p) level of theory yielded activation free energies of 17.7-20.9 kcal/mol for the initial ligation step between N,N-dialkylhydroxylamines 13″-16″ and cyclooctyne ethylcarbamate 12″ (FIG. 27A, FIG. 27B, FIG. 51). Increasing steric load on the hydroxylamine reagent was met with just a modest increase in activation barrier across the range of substrates examined (R=Me, Et, ⁱPr, ⁱBu), indicating that even the most sterically demanding tert-butylhydroxylamine 16″ should undergo rapid hydroamination at room temperature.

In contrast, hydroxylamine sterics appeared to hold dramatically more sway over product stability. Two different degradation pathways were evaluated, one involving loss of the variable alkyl substituent via Cope elimination (Path A), the other involving loss of the methoxyethylene mock linker (Path B). In each case where Path B was evaluated, it appeared inoperative at room temperature, size of the alkyl substituent notwithstanding. The activation free energy (ΔG^{‡}) for this pathway proved consistently high across substrates, 31.8 kcal/mol for the N-methyl substrate to 29.4 kcal/mol for the tert-butyl (FIG. 27B). In contrast, Path A proved more sensitive to steric environment, featuring ΔGI as low as 21.2 kcal/mol for the most sterically hindered tert-butyl substrate 18″. With an activation energy comparable to that for hydroamination, the tert-butyl substrate is likely to undergo rapid Cope elimination even at room temperature.

Consistent with the calculated Gibbs free energies, the computed ground state structure of the N-tert-butyl enamine N-oxide 18″ exhibited a significantly elongated C-N bond between the tert-butyl substituent and the N-oxide. The 1.579 Å bond length is >5% longer than the C-N bond involving the methoxyethylene appendage or either of the two N-alkyl substituents in the less sterically hindered N-methyl enamine N-oxide 17″. The long C N distance of the dissolving C-N bond in Path A is particularly notable when juxtaposed against the analogous C N distance for Path B (FIG. 27C). In aggregate, the calculations suggest that while Cope elimination is not problematic for sterically unencumbered unbranched linkers, increasing the steric environment around the enamine N-oxide significantly facilitates Cope elimination favoring loss of the larger substituent provided that a β-hydrogen is available and accessible.

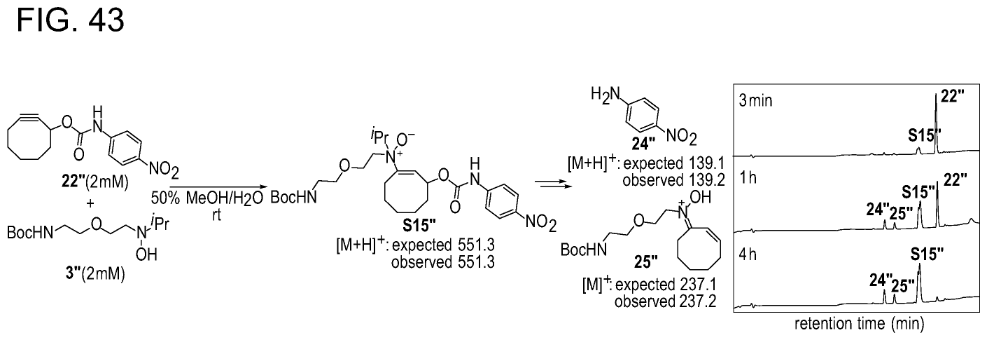

To validate these computational observations experimentally, two analogous reactions were monitored by LCMS (FIG. 27D, FIG. 43). Hydroxylamines 3″ and 4″ (2 mM) were introduced to p-nitroaniline cyclooctyne carbamate 22″ (Kang, et al., J. Am. Chem. Soc. 143:5616-5621 (2021)) (2 mM) in 50% MeOH/H₂O, and in each case, the cyclooctyne was fully and rapidly consumed over the course of 6 h. Specifically in the case of N-isopropylhydroxylamine 3″, LCMS analysis indicated the fast formation, plateauing, and persistence of the desired enamine N-oxide along with the released p-nitroaniline and the corresponding Cope elimination byproduct over that time period. Distinctly, in the case of tert-butylhydroxylamine 4″, enamine N-oxide product 23″ could not be detected by LCMS as consumption of starting material was accompanied by immediate and concomitant formation of p-nitroaniline (24″) and corresponding Cope elimination byproduct 25″. Consistent with the computational studies, Path A-based byproducts were exclusively observed for reactions involving both isopropyl and tert-butyl compounds.

The instability of enamine N-oxides 23″ and S15″ was consistent with the prior report that the hydroamination of dibenzoazacyclooctyne (DIBAC) by N,N-diethylhydroxylamine generates an unstable adduct that evades isolation (Kang, et al., J. Am. Chem. Soc. 143:5616-5621 (2021)). In contrast to the hydroamination of terminal alkynes, the bioorthogonal strain-promoted variant introduced geminal substitution on the resulting olefin with attendant A(1,2)-like strain. Consequently, cyclooctyne hydroamination appears to be much more sensitive to steric crowding around the N-oxide moiety, and degradation is significantly enhanced by alkyl branching and/or cyclooctyne arylation.

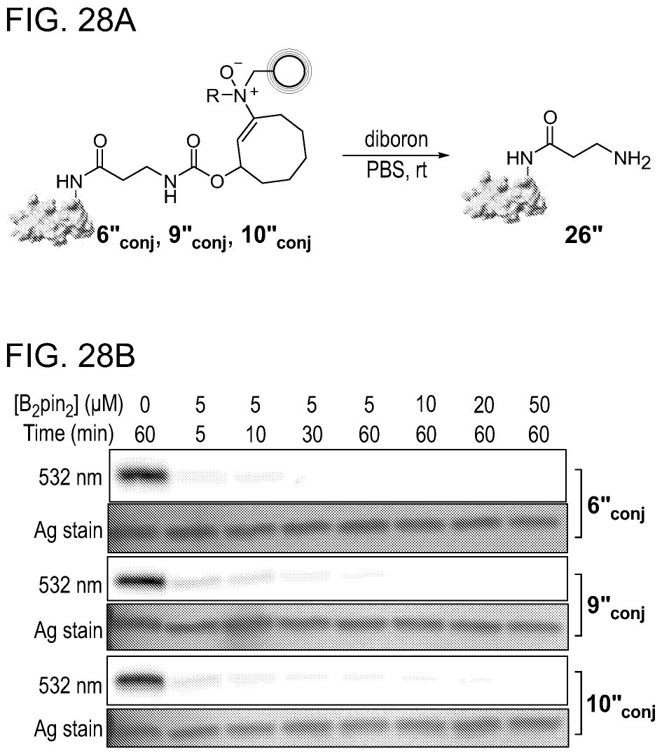

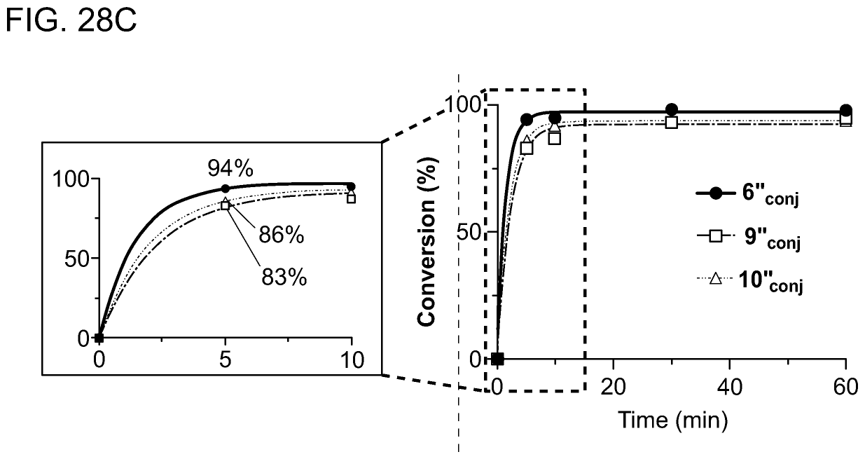

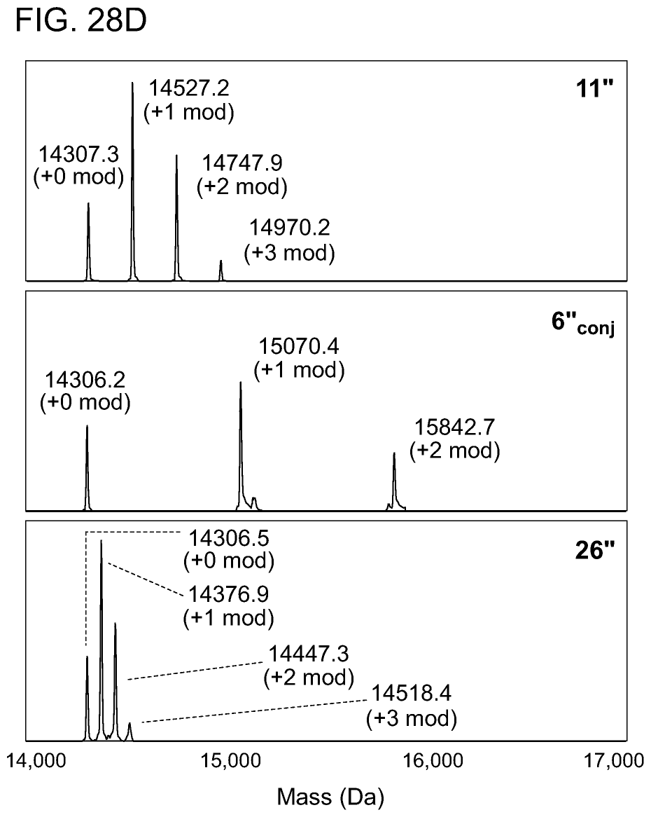

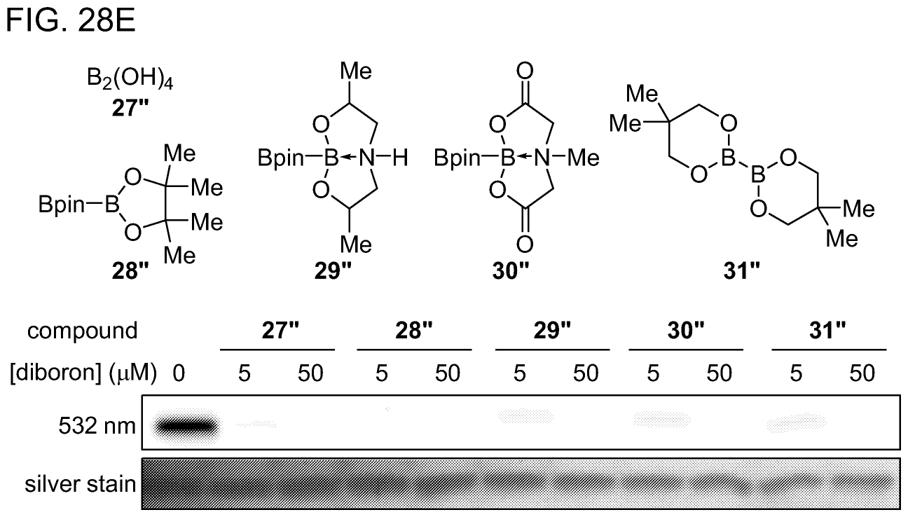

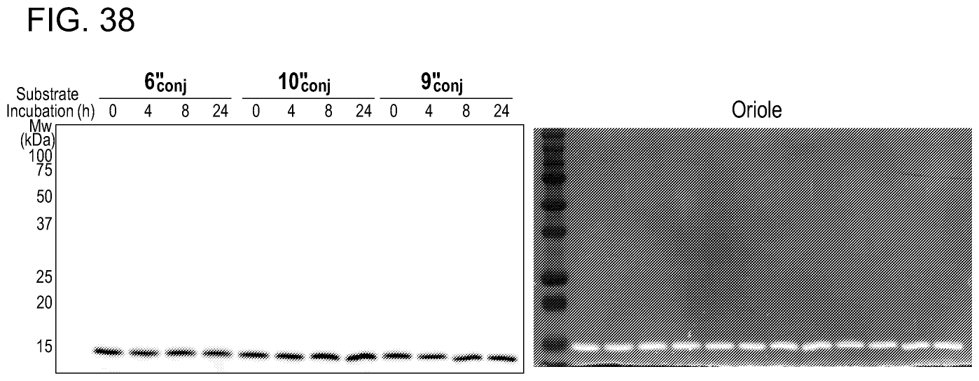

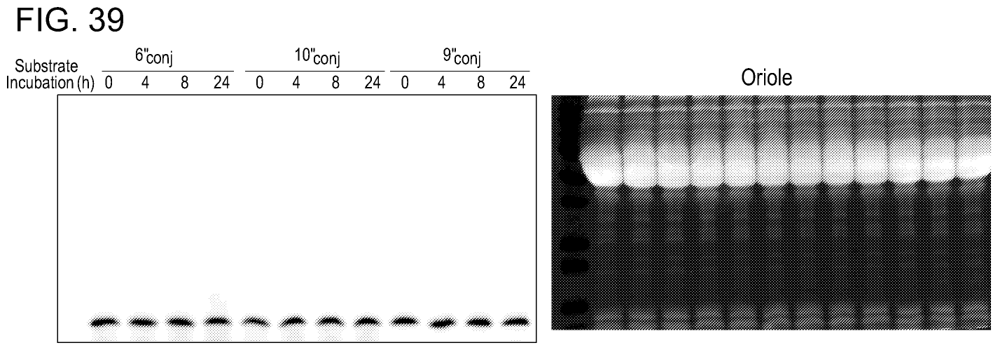

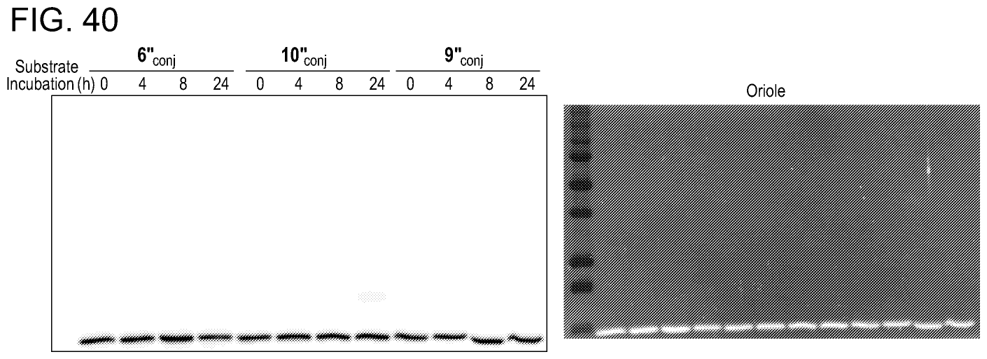

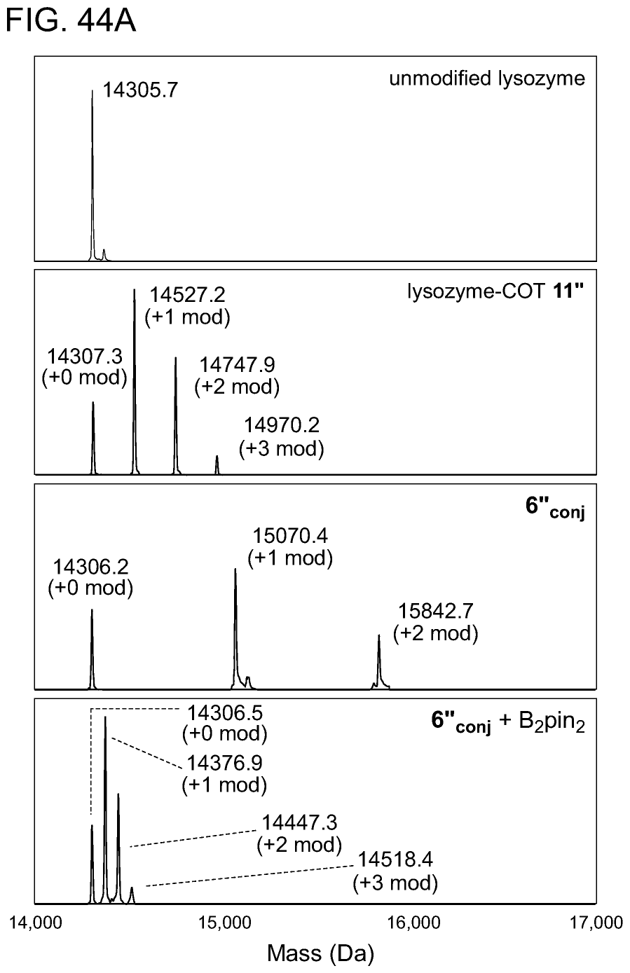

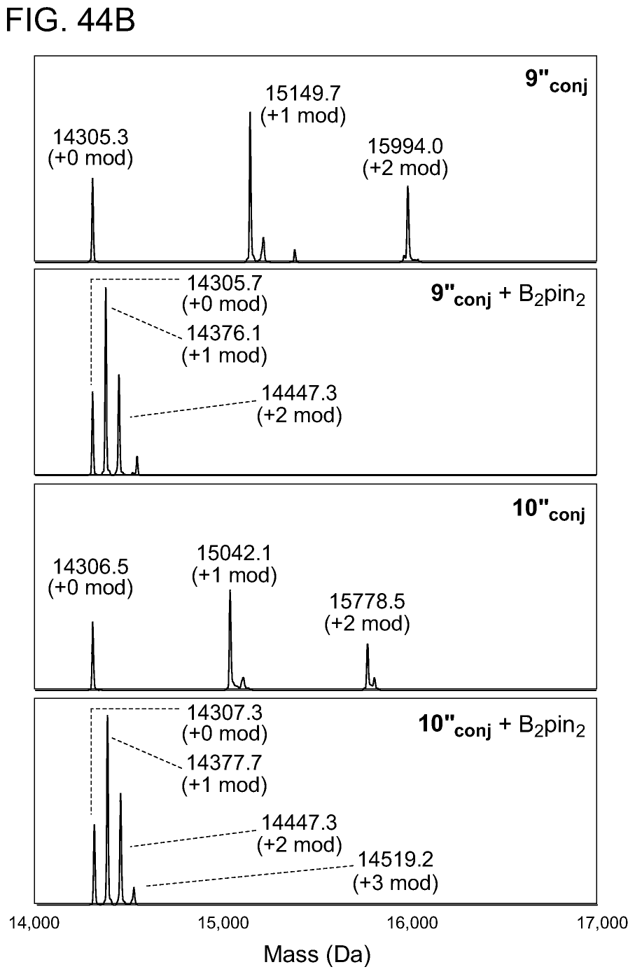

Based on these insights, the influence of substituent effects on bioorthogonal release was evaluated with reagents featuring methyl, benzyl, and piperazinyl substituents, which for lack or inaccessibility of β-hydrogens proved stable (FIG. 28A, FIG. 38-FIG. 40). Lysozyme-TAMRA conjugates 6″_{conj}, 9″_{conj}, and 10″_{conj} (480 nM) were each treated with a range of bis(pinacolato)diboron (B₂pin₂) concentrations (5-50 μM) in PBS for 1 h (Zhu, et al., Org. Lett. 14:3494-3497 (2012); Kokatla, et al., J. Org. Chem. 76:7842-7848 (2011); Carter, et al., Bifunctional Lewis Acid Reactivity of Diol-Derived Diboron Reagents. In Group 13 Chemistry/from Fundamentals to Applications; Shapiro, P. J.; Atwood, D. A., Eds.; ACS Symposium Series 822; American Chemical Society: Washington, DC; pp 70 (2002)). Complete reductive removal was observed for each substrate and concentration as indicated by the complete loss of signal in the in-gel fluorescence experiment. Reaction kinetics were then monitored by quenching the reaction at various time points with excess trimethylamine N-oxide. The efficacy of reductive cleavage was fairly general and broadly tolerant of structure (FIG. 28B). Employing 5 μM diboron, all reactions displayed >80% completion by 5 min and were observably complete by 30 min with methyl-substituted enamine N-oxide 6″_{conj} cleaving the fastest. It displayed 94% cleavage by the first time point (FIG. 28C). The quantitative nature of both click and release operations were then characterized by intact protein mass spectrometry. Cyclooctyne-lysozyme conjugate 11″, possessing 0-3 cyclooctyne linker modifications, was treated with 200 μM hydroxylamine 6″ for 6 h then cleaved with 25 μM B₂pin₂ for 30 min. ESI-MS of each transformation showed clean and complete bioorthogonal hydroamination and cleavage (FIG. 28D, FIG. 44).

The impact of boron ligands on the dissociation reaction was also explored. Ligands play a significant role in determining the physicochemical properties of boron reagents and is expected to influence the pharmacokinetics and pharmacodynamics properties of these molecules in in vivo settings. Five different diboron reagents were evaluated including unliganded tetrahydroxydiboron, diol liganded bis(pinacolato)diboron, and two mixed ligand diboron structures featuring bis(2-hydroxypropyl)amine (Gao, et al., Org. Lett. 11:3478-3481 (2009)) or methyliminodiacetic acid (Yoshida, et al., ACS Omega 2:5911-5916 (2017)) (FIG. 28E). Lysozyme-TAMRA conjugate 6″_{conj} was treated with 5 or 50 μM of each diboron reagent in PBS and analyzed by in-gel fluorescence after 1 h. It was discovered that the diboron-induced cleavage of enamine N-oxides is relatively agnostic to the ligand, tolerating even the most sterically demanding bidentate and tridentate ligands. Complete cleavage was observed for all reagents at 50 μM concentrations. Tetrahydroxydiboron and bis(pinacolato)diboron stood out amongst the five, displaying the greatest reactivities and allowing the reaction to reach completion by 1 h even at 5 μM concentrations. While just perceptibly incomplete, the other three reactions were still rapid, exhibiting >95% completion at the same concentration in 1 h.

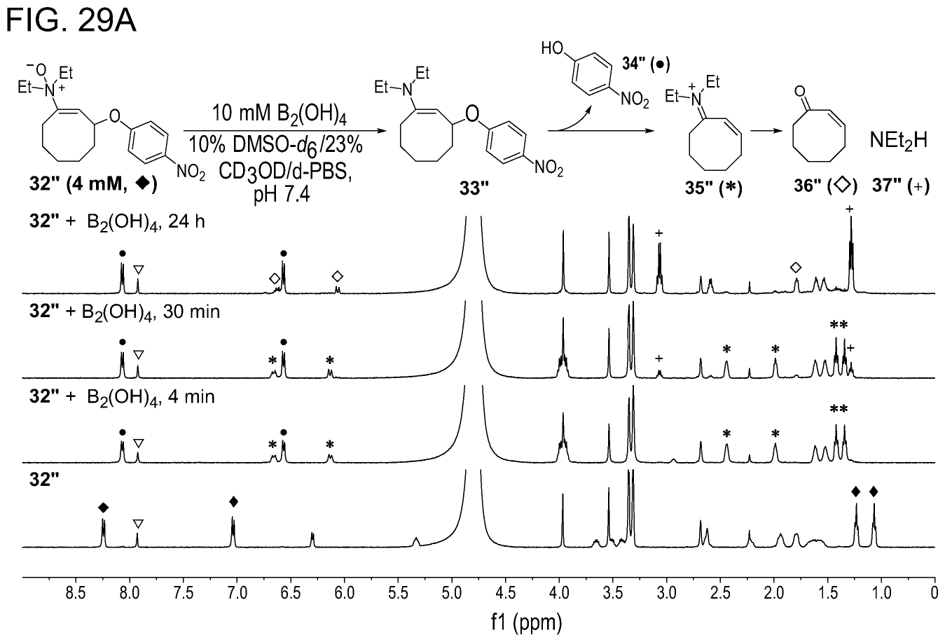

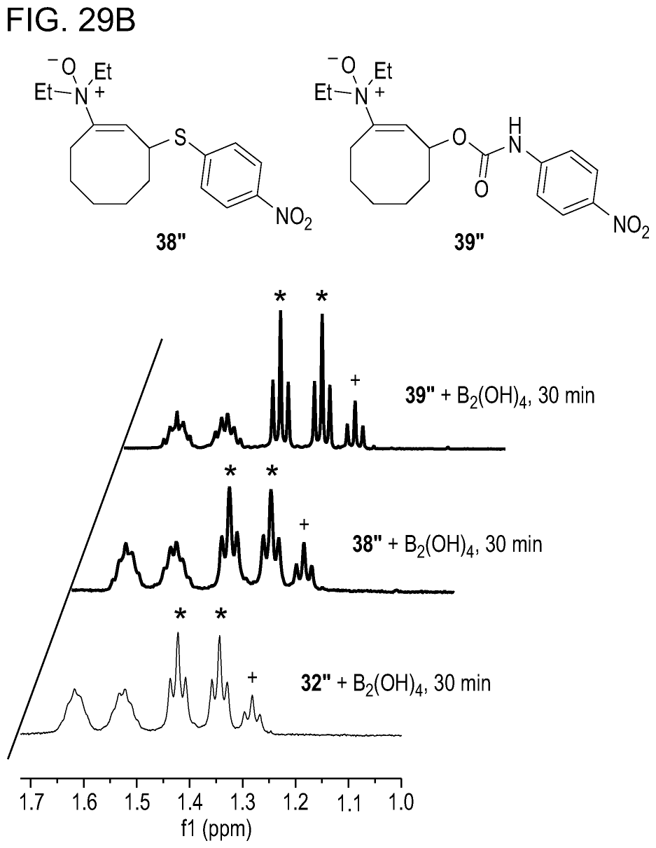

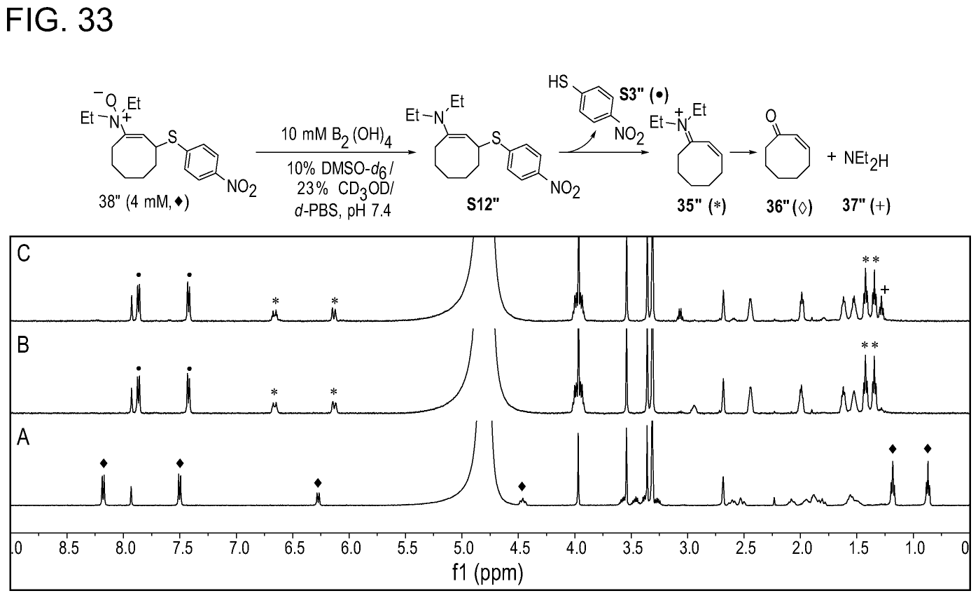

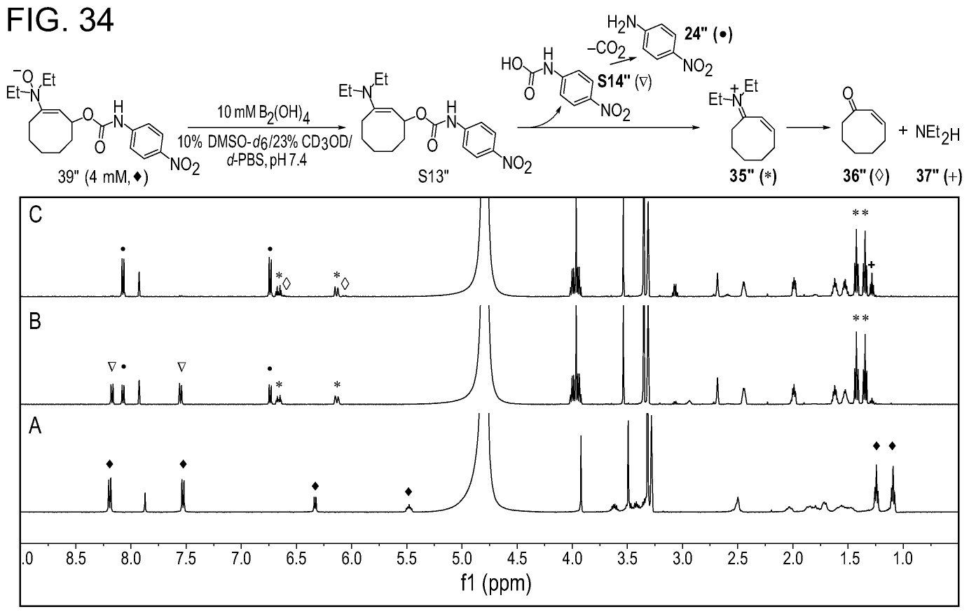

Diboron reagents and enamine N-oxide structures in hand, the reaction kinetics were characterized and the substrate scope for the cleavage reaction was explored. A series of chromogenic probes 32″, 38″, 39″ featuring a representative series of nitrogen, oxygen, and sulfur-bearing leaving groups were obtained. Each probe was synthesized from cyclooct-2-ynol either by Mitsunobu reaction or carbamoylation with the corresponding p-nitro(thio)phenol or p-nitrophenyl isocyanate followed by hydroamination with N,N-diethylhydroxylamine. The formation of the intended products was verified by ¹H NMR spectroscopy with caffeine as an internal standard (FIG. 29A, FIG. 33, FIG. 34). When p-nitrophenyl ether 32″ was treated with 10 mM B₂(OH)₄ in 10% DMSO-d₆/23% CD₃OD/d-PBS, pH 7.4, the first spectrum obtained at 4 min demonstrated both complete reduction of the N-oxide as well as quantitative formation of the released payload p-nitrophenol (34″) together with the α,β-unsaturated iminium ion 35″. Over the course of 24 h, iminium ion 35″ hydrolyzed to cyclooctenone 36″ and diethylamine 37″. The other substrates reacted analogously with the same quantitative formation of iminium ion 35″ at the initial time point (FIG. 29B). Importantly, while it could not be ascertained whether reduction or elimination were rate limiting under these conditions, the experiment placed an upper limit on the half-life of enamine 33″ at a few minutes.

The chromogenic probes that were prepared were intended for stopped flow kinetics experiments using UV-vis spectroscopy; however, the NMR studies precluded the use of one of these compounds for this purpose. Specifically, carbamate 39″ resulted in release of 4-nitrophenylcarbamic acid, which persisted as a discrete intermediate on a timescale longer than that for either reduction or release before undergoing decarboxylation. Since a change in UV absorbance is contingent on p-nitroaniline formation, it could not be used or any analogous carbamate-based chromogenic or fluorogenic outputs as accurate proxies for product release. Instead, a fluorescence polarization assay was employed. Fluorescence polarization measurements are directly responsive to the presence or absence of a direct interaction between protein and small molecule and are capable of reporting on a bond cleavage event with great fidelity. Furthermore, performing the cleavage reaction on proteins would provide an accurate representation of the reaction kinetics in a biological setting.

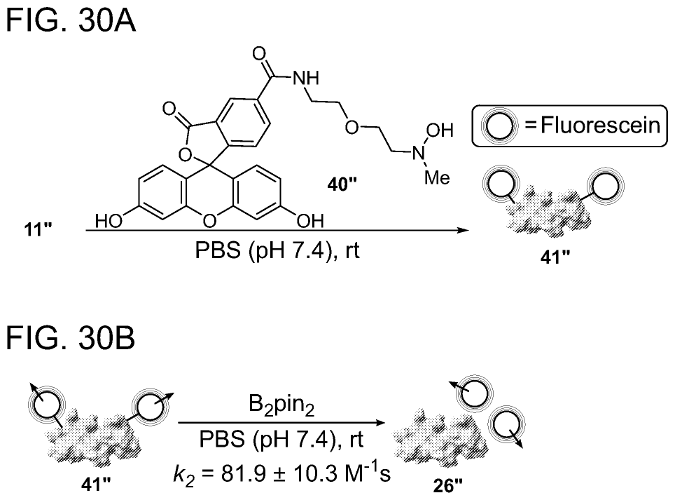

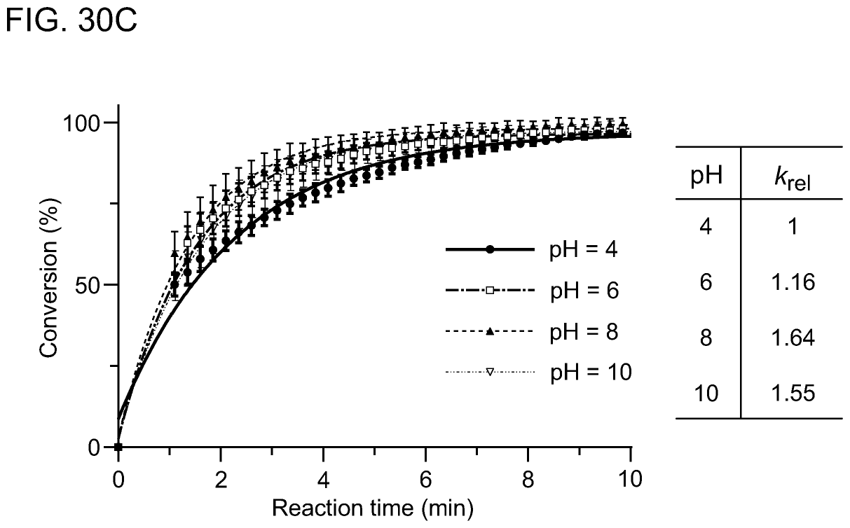

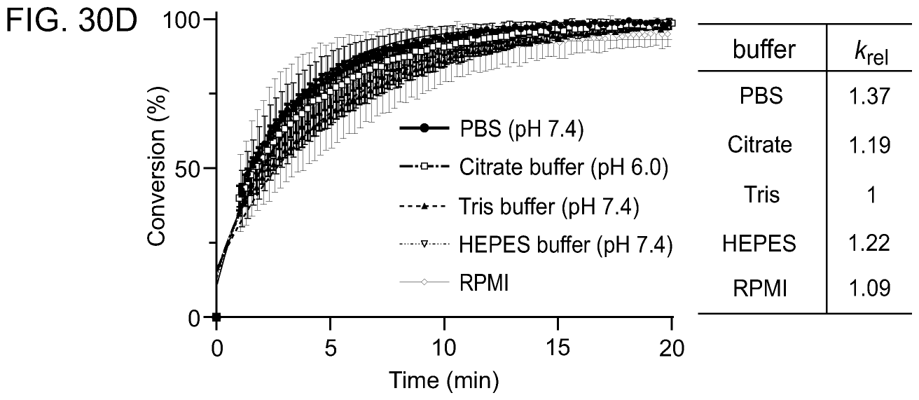

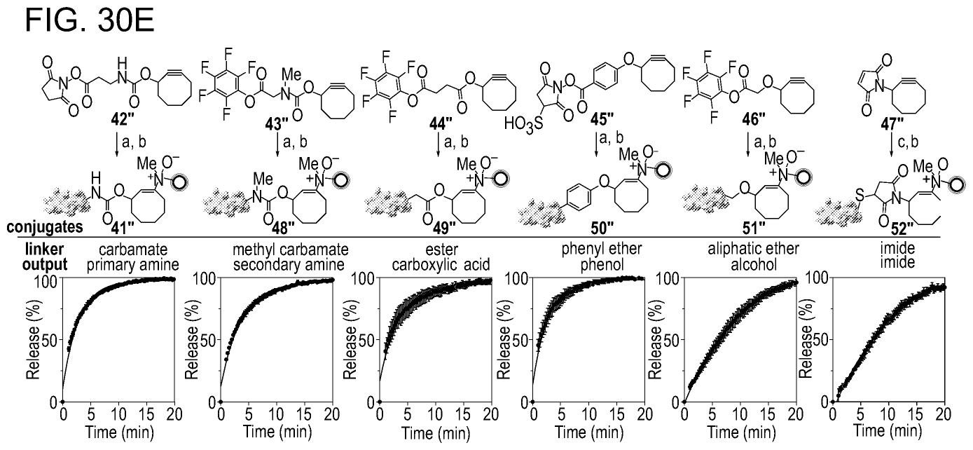

To this end, hydroxylamine-linked fluorescein 40″ was synthesized and used it to functionalize cyclooctyne-lysozyme conjugate 11″ by retro-Cope elimination (FIG. 30A). 500 nM fluorescein-lysozyme conjugate 41″ was then treated with excess B₂pin₂ (25-200 μM) in PBS, pH 7.4, at room temperature, and determined that the reaction rate is first order in diboron reagent at this concentration range. The second order rate constant for this reaction was found to be 81.9 M⁻¹s⁻¹ (FIG. 30B). The kinetics of the reaction at various pH's was examined to determine whether N-oxide protonation under acidic conditions or diboronate formation under basic conditions would adversely impact reactivity. Fortunately, the reaction was found to be relatively insensitive to solvent pH in the examined range and the reaction to be only marginally faster at pH 10 than at pH 4 (FIG. 30C). The diboron-mediated cleavage is also compatible with a full range of common aqueous buffers including PBS (pH 7.4), citrate buffer (pH 6.0), Tris buffer (pH 7.4), HEPES buffer (pH 7.4), and RPMI growth medium. Importantly, buffer content had minimal impact on reactivity, testifying to the generality of this method. Irrespective of the specific solvent conditions, all reactions were >99% complete within 5-20 min when 50 μM B₂pin₂ was employed (FIG. 30D).

To further demonstrate the versatility of the diboron-mediated dissociative transformation, the bond cleavage kinetics for products linked by different functional groups was evaluated. Cyclooctynes 42″-47″ featuring primary and secondary amine carbamates, an ester, phenyl and alkyl ethers, and an imide as leaving groups were synthesized (FIG. 30E). With the exception of imide 47″ (Hagendorn, et al., Eur. J. Org. Chem. 2014:1280-1286 (2014)), which was conjugated to a cysteine residue by Michael addition, each of these compounds were attached to lysine residues on lysozyme via activated (sulfo)NHS or pentafluorophenyl esters. Fluorescein hydroxylamine 40″ was subsequently ligated onto these cyclooctynes by hydroamination to generate enamine N-oxide-linked adducts, which were then reduced using 50 μM B₂pin₂ in PBS, pH 7.4. Progress of payload release was monitored by fluorescence polarization. The reaction profiles were nearly identical for substrates 41″, 48″-50″. It was already determined above that N-oxide reduction is rate limiting for the primary amine carbamate 41″ at these diboron concentrations; this is likely true of the other three substrates containing activated leaving groups. The reaction kinetics were rapid with each reaction achieving >90% product release within 10 min and full conversion being obtained by 20 min. In contrast, it was observed that the less activated alkyl alcohol and imide leaving groups displayed a markedly different reaction profile reflective of their slower release rates and likely shifted in the rate determining step. Nonetheless, despite the slower elimination rates, alkyl alcohols and imides also exhibited >96% and 92% product release within 20 min, respectively.

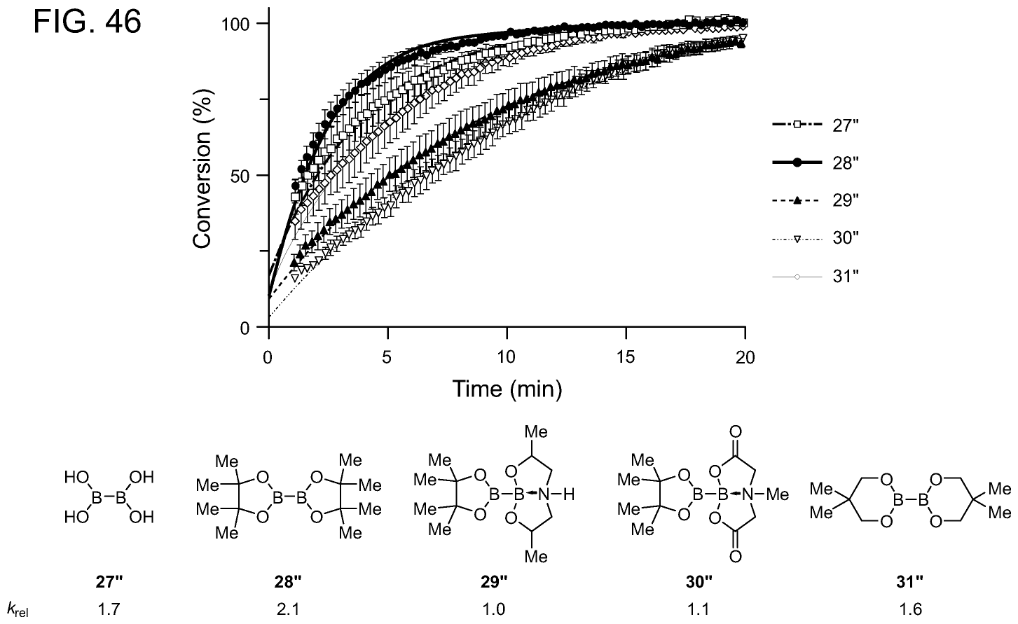

Finally, the influence of diboron structure on the rate of enamine N-oxide reduction for diboron reagents 27″-31″ was corroborated using the same fluorescence polarization-based kinetics assay (FIG. 46).

Having developed the independent components of click and release, the components were integrated in an application involving the ligation and cleavage of a small molecule from a protein. Antibody-drug conjugates presented the perfect platform as dissociative reactions provide a chemically induced mechanism of drug release independent of cellular catabolic processes. The work highlights how both parts of the reactions can be used for forming and cleaving these conjugates.

The work commenced with the synthesis of hydroxylamine-bearing antibodies. 1-Hydroxypiperazine 54″ was rapidly synthesized by alkylation of N-Boc-piperazine (53″) with acrylonitrile in methanol followed by N-oxidation and Cope elimination of the resulting N-oxide.

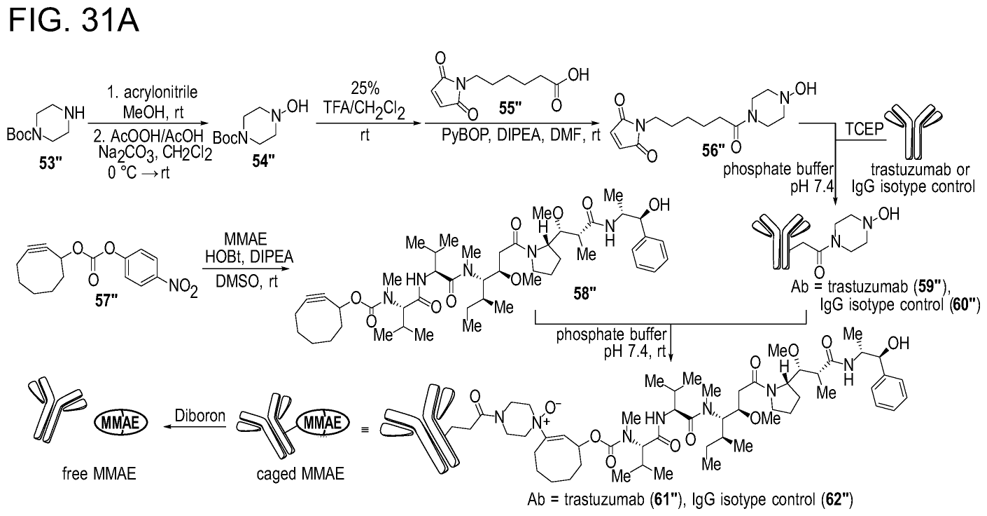

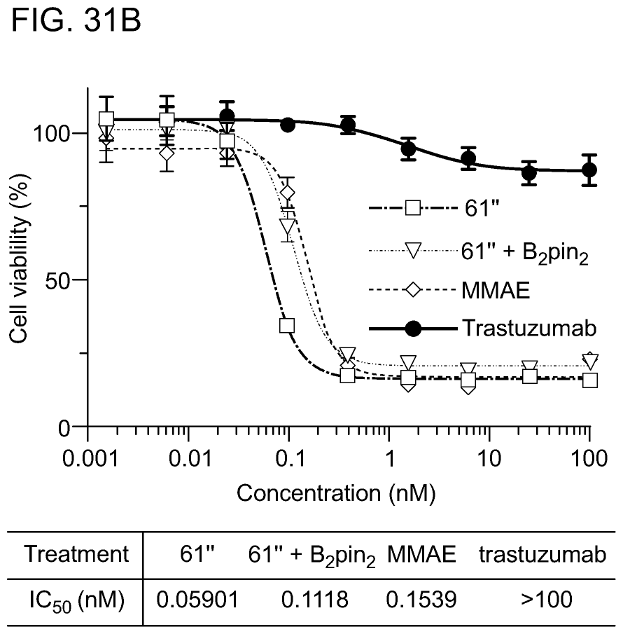

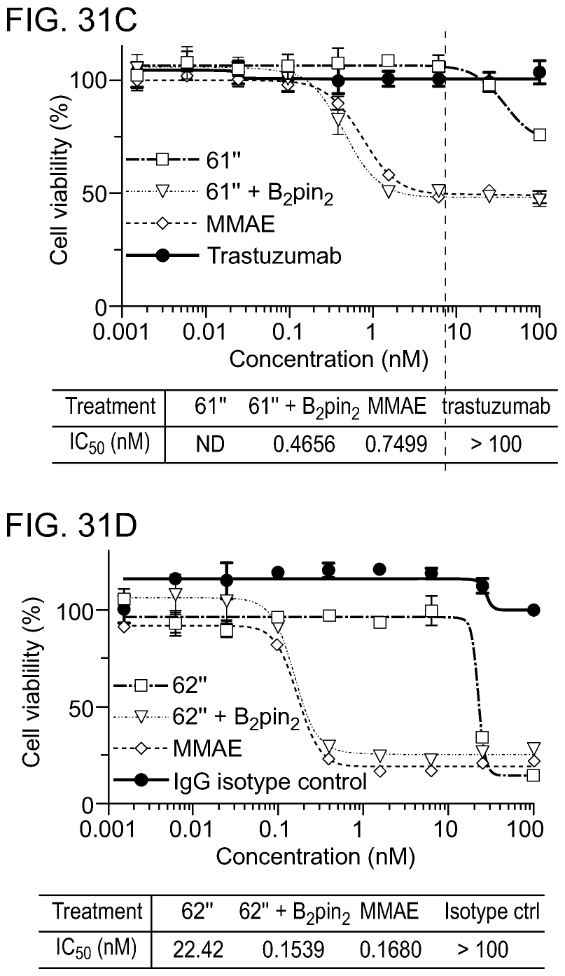

Trifluoroacetic acid-mediated Boc deprotection and PyBOP-mediated amide coupling with 6-maleimidohexanoic acid (55″) produced maleimide 56″, which could be appended to either trastuzumab or human IgG isotype control antibodies by conjugate addition of cysteine residues generated by TCEP-mediated reduction of the hinge disulfides on IgG. Separately, cyclooctyne-modified cytotoxin monomethyl auristatin E (MMAE-OCT, 58″) was obtained by carbamylation of MMAE with cyclooctynyl p-nitrophenyl carbonate 57″. With each component in hand, the ligation of MMAE-OCT onto either 1-hydroxypiperazine-modified trastuzumab 59″ or IgG isotype control 60″ was carried out by bioorthogonal hydroamination in PBS at room temperature to afford ADCs 61″ and 62″, respectively (FIG. 31A).

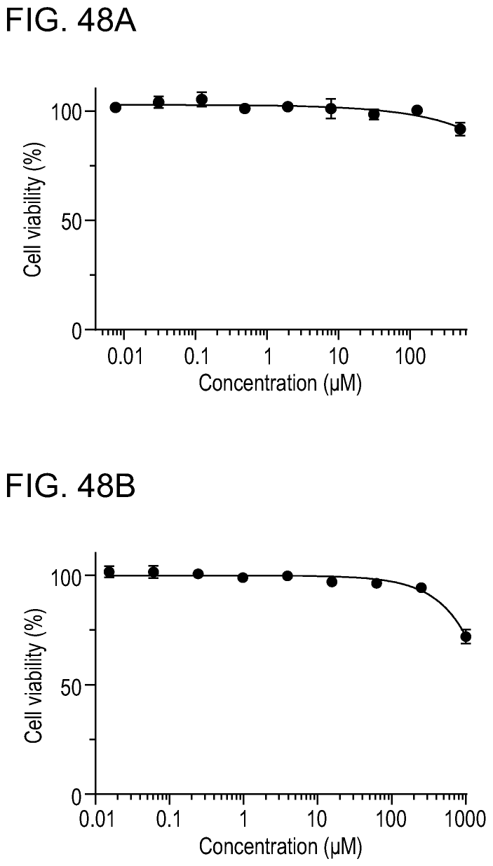

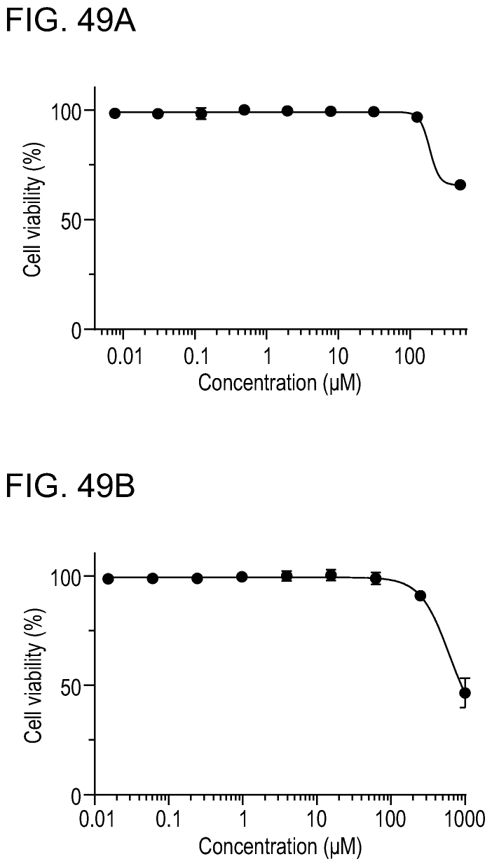

After confirming the cellular compatibility of our diboron reagents (IC₅₀>500 μM, FIG. 48, FIG. 49) and verifying the stability of piperazinyl enamine N-oxide-linked IgG conjugates in both RPMI+5% human serum (Table 3) and conditioned media from SK-BR-3 breast cancer cell lines (<3% release over 42 h, Table 4), the efficacy of the chemistry in inducing drug release from ADCs was evaluated by measuring its impact on cell viability. SK-BR-3 cells were treated with 1.5 μM-100 nM trastuzumab-MMAE 61″ in the presence or absence of 50 μM B₂pin₂, cultured for 72 h, and assayed using the CellTiter-Glo® cell viability assay. The IC₅₀ of the ADC was insensitive to the presence or absence of diboron and recapitulated the IC₅₀ of MMAE alone. The IC₅₀ of ADC 61″ alone (0.05901 nM) was comparable to that of ADC with diboron (0.1118 nM). Each recapitulated the toxicity of MMAE alone (IC₅₀=0.1539 nM). Unmodified trastuzumab was found to have little activity at these concentrations (FIG. 31B).

In contrast, when the same experiment was performed on the triple negative breast cancer cell line MDA-MB-231 lacking HER2 amplification, a marked difference was observed in the cell viability curves after 96 h for trastuzumab-MMAE 61″ alone or in combination with 50 μM diboron (IC₅₀=0.4656 nM). Only when diboron was employed did the ADC reproduce the toxicity of MMAE (IC₅₀=0.7499 nM) (FIG. 31C). As further control, trastuzumab was replaced with a human IgG isotype control, employing ADC 62″. Unable to undergo receptor-mediated internalization and drug release in the SK-BR-3 cell line, the ADC exhibited a 145-fold enhancement in cell toxicity when used in combination with the diboron reagent versus without. Importantly, the diboron-induced drug release mechanism exhibited identical effects on cell viability as MMAE alone consistent with complete release of drug (FIG. 31D). This N-oxide-based drug delivery platform provides a convenient mechanism for loading drugs onto antibodies as well as an appealing alternative to existing methods for the fast and complete release of drug molecules from their carriers.

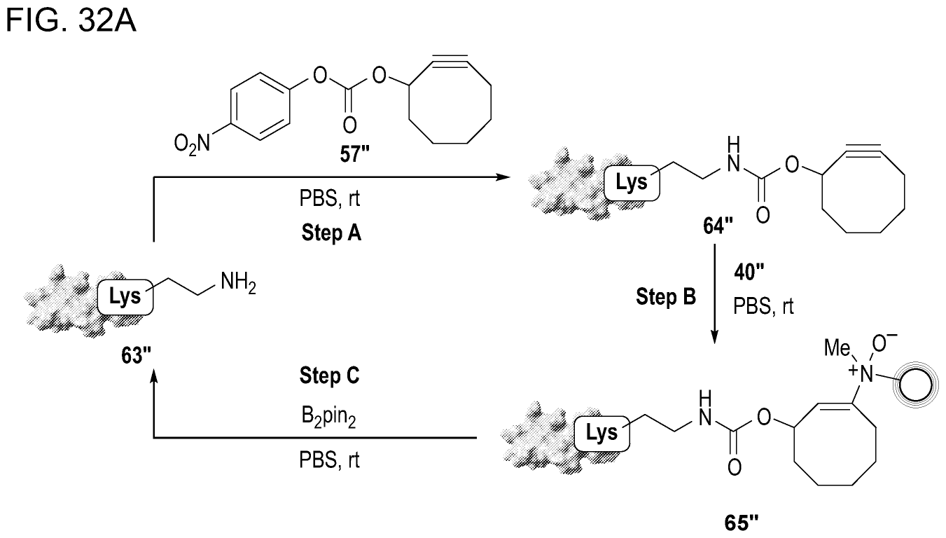

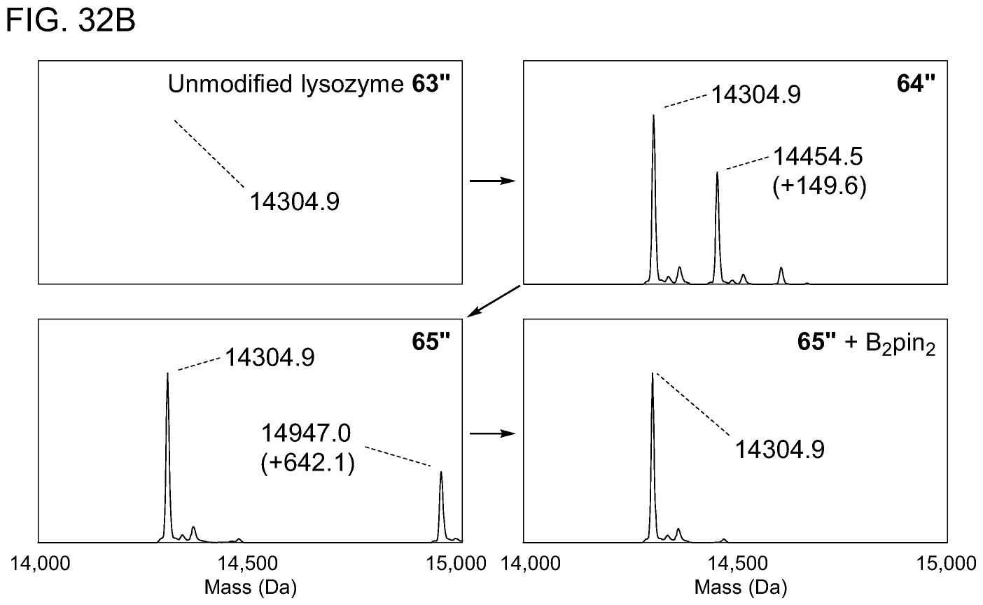

The chemically reversible bioorthogonal reaction that has been described is both directional and traceless. In the antibody-drug conjugate application, the quantitative release of the small molecule MMAE was demonstrated. There, the drug was released in its native form without derivatization. When the traceless modification of a protein is desired instead, it is possible to easily reverse the polarity of the chemical handles to remove any residual modifications on the protein. This powerful feature is demonstrated through the reversible functionalization of lysozyme (FIG. 32A).

Lysozyme was first modified with a cyclooctyne using cylooctynyl p-nitrophenylcarbonate 57″ to afford the cyclooctyne-modified protein 64″, which was suitable for bioconjugation. In this proof-of-principle experiment, fluorescein was conjugated via the corresponding hydroxylamine 40″. Finally, both the fluorescein and cyclooctyne handle could be removed completely using 25 μM B₂pin₂ in PBS to restore the original lysine residue. The traceless sequence of chemical operations was verified by ESI-MS (FIG. 32B). Although lysozyme was modified by reaction with carbonate 57″ in this particular example, the described method of traceless click and release is agnostic to the method of cyclooctyne incorporation. When combined with existing methods of site-specific incorporation such as with unnatural amino acids (Nikic, et al., Nat. Protoc. 10:780-791 (2015)), this bioorthogonal reaction sequence can potently enable the precise modification and manipulation of proteins.

### Example 68: Synthesis of tert-Butyl (2-(2-(hydroxy(isopropyl)amino)ethoxy)ethyl)carbamate (3″)

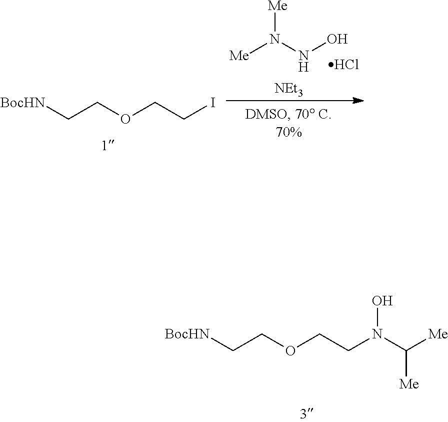 [SMILES: CN(C)NO ; Cl ; CC(C)(C)OC(=O)NCCOCCI ; CC(C)N(O)CCOCCNC(=O)OC(C)(C)C ; "NEt3"]

N-Isopropylhydroxylamine hydrochloride (354 mg, 3.17 mmol) and triethylamine (884 μL, 6.35 mmol) were added sequentially to a solution of alkyl iodide 1″ (Kang, et al., J. Am. Chem. Soc. 143:5616-5621 (2021)) (500 mg, 1.59 mmol) in dimethylsulfoxide (1.59 mL). The reaction mixture was stirred at 70° C. for 1.5 h, then the resulting mixture was diluted with water and purified by automated C₁₈ reverse phase column chromatography (30 g C₁₈ silica gel, 25 μm spherical particles, eluent: H₂O+0.1% TFA (5 CV), gradient 0→100% MeCN/H₂O+0.1% TFA (10 CV)). Fractions containing the desired product were collected and concentrated under reduced pressure. The resulting residue was then purified by flash column chromatography on silica gel (eluent: 5% CMA in chloroform) to afford the title compound as a colorless oil (342 mg, 70%). TLC (10% CMA in chloroform), Rf: 0.17 (I₂). ¹H NMR (500 MHz, CD₃OD, 25° C.) δ 3.64 (t, J=5.7 Hz, 2H), 3.49 (t, J=5.5 Hz, 2H), 3.23 (t, J=5.5 Hz, 2H), 2.92-2.77 (m, 3H), 1.44 (s, 9H), 1.10 (d, J=6.5 Hz, 6H). ¹³C NMR (126 MHz, CD₃OD, 25° C.) δ 158.4, 80.0, 71.1, 69.5, 59.2, 56.9, 41.4, 29.0, 18.9. FTIR (thin film) cm⁻¹: 3355 (br), 2974 (m), 2933 (w), 2874 (w), 1692 (s), 1521 (m), 1390 (m), 1274 (m), 1249 (m), 1170 (s), 1122 (s). HRMS (ESI) (m/z): calc'd for C₁₂H₂₇N₂O₄ [M+H]⁺: 263.1965, found: 263.1964.

### Example 69: Synthesis of 3′,6′-Bis(dimethylamino)-N-(2-(2-(hydroxy(isopropyl)amino)ethoxy)ethyl)-3-oxo-3H-spiro[isobenzofuran-1,9′-xanthene]-6-carboxamide (7″)

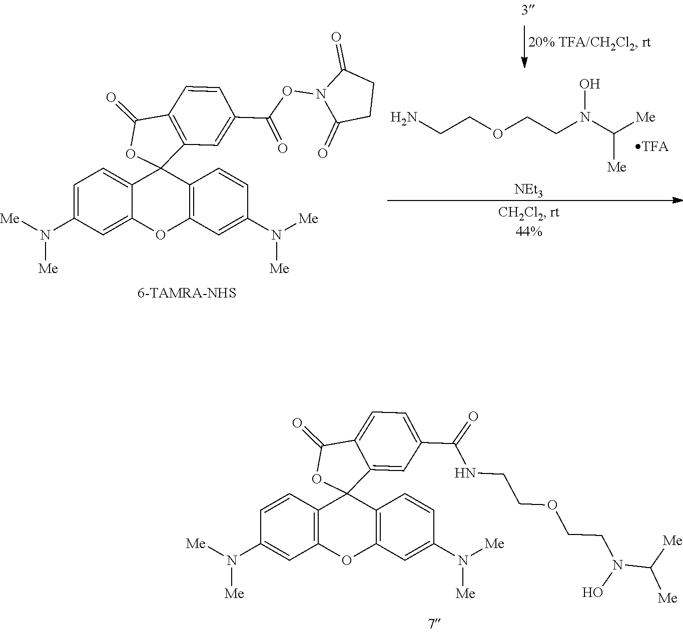

Trifluoroacetic acid (200 μL) was added to a solution of hydroxylamine 3″ (11.4 mg, 43.5 μmol) in dichloromethane (800 μL). The resulting solution was stirred at room temperature for 30 min then concentrated under reduced pressure. The resulting residue was dissolved in dichloromethane (1.0 mL), and triethylamine (20.2 μL, 145 μmol) and 6-carboxytetramethylrhodamine N-succinimidyl ester (6-TAMRA-NHS, 15.3 mg, 29.0 μmol) were sequentially added to the solution. The reaction mixture was stirred at room temperature for 4 h, concentrated under reduced pressure, and purified by automated C₁₈ reverse phase column chromatography (30 g C₁₈ silica gel, 25 μm spherical particles, eluent: H₂O+0.1% TFA (5 CV), gradient 0→100% MeCN/H₂O+0.1% TFA (15 CV)) and flash column chromatography on silica gel (eluent: 70% CMA in chloroform) to afford the title compound as a violet solid (7.3 mg, 44%). TLC (70% CMA in chloroform), Rf: 0.35 (UV). ¹H NMR (500 MHz, CD₃OD, 25° C.) δ 8.15 (d, J=8.1 Hz, 1H), 8.09 (dd, J=8.1, 1.8 Hz, 1H), 7.73 (d, J=1.8 Hz, 1H), 7.22 (d, J=9.4 Hz, 2H), 7.01 (dd, J=9.5, 2.5 Hz, 2H), 6.92 (d, J=2.5 Hz, 2H), 3.66 (t, J=5.5 Hz, 2H), 3.60 (t, J=5.2 Hz, 2H), 3.54 (t, J=5.2 Hz, 2H), 3.28 (s, 12H), 2.92-2.83 (m, 3H), 1.02 (d, J=6.4 Hz, 6H). ¹³C NMR (126 MHz, CD₃OD, 25° C.) δ 172.5, 168.8, 162.0, 159.2, 158.9, 143.8, 136.8, 134.3, 132.8, 131.4, 129.8, 129.7, 115.2, 115.1, 97.5, 70.5, 69.1, 59.5, 56.9, 41.2, 41.0, 18.5. FTIR (thin film) cm⁻¹: 3283 (br) 2930 (w), 1648 (m), 1592 (s), 1491 (m), 1349 (s), 1189 (s). HRMS (ESI) (m/z): calc'd for C₃₂H₃₉N₄O₆ [M+H]⁺: 575.2864, found: 575.2855.

### Example 70: Synthesis of tert-Butyl (2-(2-(tert-butyl(hydroxy)amino)ethoxy)ethyl)carbamate (4″)

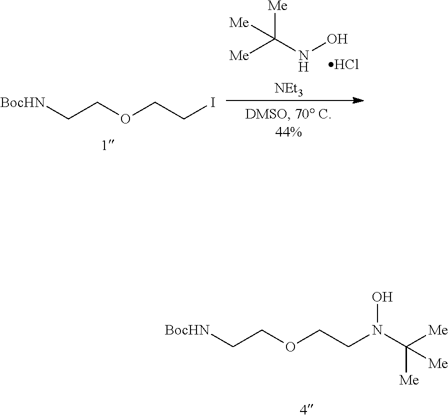 [SMILES: CC(C)(C)NO ; Cl ; CC(C)(C)OC(=O)NCCOCCI ; CC(C)(C)OC(=O)NCCOCCN(O)C(C)(C)C ; "NEt3"]

N-(tert-Butyl)hydroxylamine hydrochloride (398 mg, 3.17 mmol) and triethylamine (884 μL, 6.35 mmol) were added sequentially to a solution of alkyl iodide 1″ (500 mg, 1.59 mmol) in dimethylsulfoxide (1.59 mL). The reaction mixture was stirred at 70° C. for 1.5 h, then the resulting mixture was diluted with water and purified by automated C₁s reverse phase column chromatography (30 g C₁s silica gel, 25 μm spherical particles, eluent: H₂O+0.1% TFA (5 CV), gradient 0→100% MeCN/H₂O+0.1% TFA (10 CV)). Fractions containing the desired product were collected and concentrated under reduced pressure. The resulting residue was then purified by flash column chromatography on silica gel (eluent: 5% CMA in chloroform) to afford the title compound as a colorless oil (193 mg, 44%). TLC (10% CMA in chloroform), Rf: 0.30 (UV, I₂). ¹H NMR (500 MHz, CD₃OD, 25° C.) δ 3.64 (t, J=5.7 Hz, 2H), 3.50 (t, J=5.5 Hz, 2H), 3.23 (t, J=5.5 Hz, 2H), 2.82 (t, J=5.9 Hz, 2H), 1.44 (s, 9H), 1.11 (s, 9H). ¹³C NMR (126 MHz, CD₃OD, 25° C.) δ 158.5, 80.1, 71.0, 70.2, 59.8, 52.9, 41.4, 29.0, 25.6. FTIR (thin film) cm⁻¹: 3362 (br), 2974 (m), 1692 (s), 1513 (m), 1390 (m), 1249 (m), 1170 (s), 1118 (s). HRMS (ESI) (m/z): calc'd for C₁₃H₂₉N₂O₄ [M+H]⁺: 277.2122, found: 277.2120.

### Example 71: Synthesis of N-(2-(2-(tert-Butyl(hydroxy)amino)ethoxy)ethyl)-3′,6′-bis(dimethylamino)-3-oxo-3H-spiro[isobenzofuran-1,9′-xanthene]-6-carboxamide (8″)

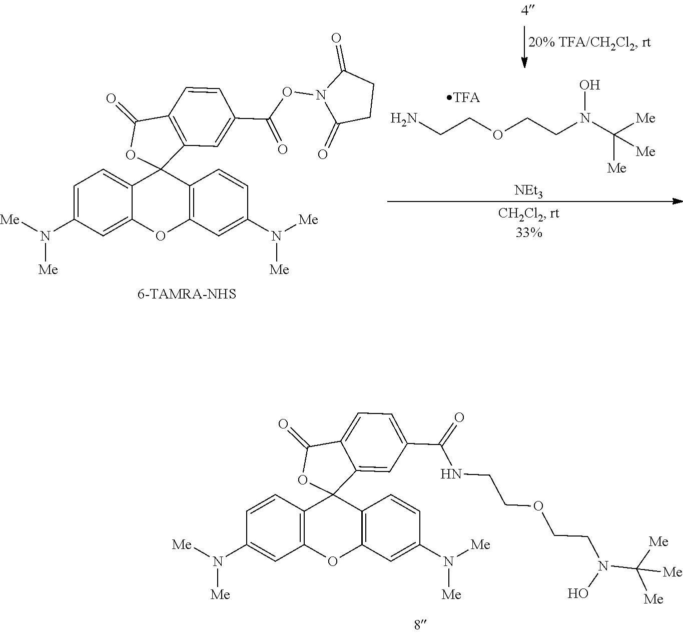

Trifluoroacetic acid (200 μL) was added to a solution of hydroxylamine 4″ (15.6 mg, 53.6 μmol) in dichloromethane (800 μL). The resulting solution was stirred at room temperature for 45 min then concentrated under reduced pressure. The resulting residue was dissolved in dichloromethane (1.0 mL), and triethylamine (26.1 μL, 188 μmol) and 6-TAMRA-NHS (19.8 mg, 37.5 μmol) were sequentially added to the solution. The reaction mixture was stirred at room temperature for 1.5 h, concentrated under reduced pressure, and purified by automated C₁₈ reverse phase column chromatography (30 g C₁₈ silica gel, 25 μm spherical particles, eluent: H₂O+0.1% TFA (5 CV), gradient 0→100% MeCN/H₂O+0.1% TFA (15 CV)) and flash column chromatography on silica gel (eluent: 70% CMA in chloroform) to afford the title compound as a violet solid (7.3 mg, 33%). TLC (70% CMA in chloroform), Rf: 0.24 (UV). ¹H NMR (500 MHz, CD₃OD, 25° C.) δ 8.14 (d, J=8.1 Hz, 1H), 8.10 (dd, J=8.1, 1.8 Hz, 1H), 7.73 (d, J=1.8 Hz, 1H), 7.24 (d, J=9.5 Hz, 2H), 7.01 (dd, J=9.5, 2.5 Hz, 2H), 6.92 (d, J=2.5 Hz, 2H), 3.67-3.59 (m, 4H), 3.56 (t, J=5.1 Hz, 2H), 3.28 (s, 12H), 2.83 (s, 2H), 1.03 (s, 9H). ¹³C NMR (126 MHz, CD₃OD, 25° C.) δ 172.6, 168.9, 162.1, 159.2, 158.9, 144.3, 136.6, 134.2, 132.9, 131.3, 129.8, 129.7, 115.2, 115.1, 97.5, 70.4, 70.0, 60.4, 53.1, 41.3, 41.0, 25.4. FTIR (thin film) cm⁻¹: 3288 (br), 2971 (w), 1648 (w), 1595 (s), 1491 (m), 1349 (s), 1189 (s). HRMS (ESI) (m/z): calc'd for C₃₃H₄₁N₄O₆ [M+H]⁺: 589.3021, found: 589.3008.

### Example 72: Synthesis of tert-Butyl (2-(2-(benzyl(hydroxy)amino)ethoxy)ethyl)carbamate (5″)

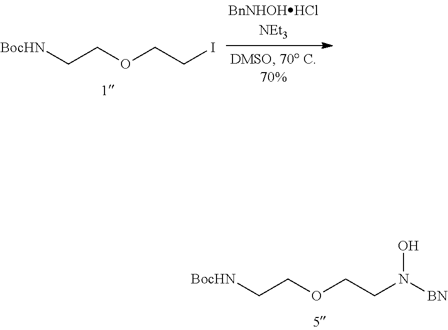

Triethylamine (884 uL, 6.35 mmol) was added to a solution of alkyl iodide 1″ (500 mg, 1.59 mmol) and N-benzylhydroxylamine hydrochloride (506 mg, 3.17 mmol) in dimethylsulfoxide (1.59 mL) at room temperature. The reaction mixture was then heated to 70° C. After 1.5 h, the solution was cooled to room temperature, diluted with water, and purified by automated C₁s reverse phase column chromatography (30 g C₁s silica gel, 25 μm spherical particles, eluent: H₂O+0.1% TFA (5 CV), gradient 0→100% MeCN/H₂O+0.1% TFA (15 CV)). Fractions containing the desired product were collected and concentrated under reduced pressure. The resulting residue was then purified by flash column chromatography on silica gel (eluent: 5% CMA in chloroform) to afford the title compound as a colorless oil (342 mg, 70%). TLC (10% CMA in chloroform), Rf 0.33 (I₂). ¹H NMR (500 MHz, CD₃OD, 25° C.) δ 7.41-7.34 (m, 2H), 7.33-7.27 (m, 2H), 7.27-7.20 (m, 1H), 3.83 (s, 2H), 3.64 (t, J=5.6 Hz, 2H), 3.47 (t, J=5.5 Hz, 2H), 3.21 (t, J=5.4 Hz, 2H), 2.87 (t, J=5.6 Hz, 2H), 1.43 (s, 9H). ¹³C NMR (126 MHz, CD₃OD, 25° C.) δ 158.3, 138.8, 130.9, 129.2, 128.3, 80.1, 71.0, 69.2, 66.3, 60.5, 41.3, 28.9. FTIR (thin film) cm⁻¹: 3355 (br), 2974 (w), 2929 (w), 2870 (w), 1692 (s), 1513 (m), 1249 (m), 1167 (s), 1118 (s). HIRMS (ESI) (m/z): calc'd for C₁₆H₂₇N₂O₄ [M+H]⁺: 311.1965, found: 311.1961.

### Example 73: Synthesis of N-(2-(2-(Benzyl(hydroxy)amino)ethoxy)ethyl)-3′,6′-bis(dimethylamino)-3-oxo-3H-spiro[isobenzofuran-1,9′-xanthene]-6-carboxamide (9″)

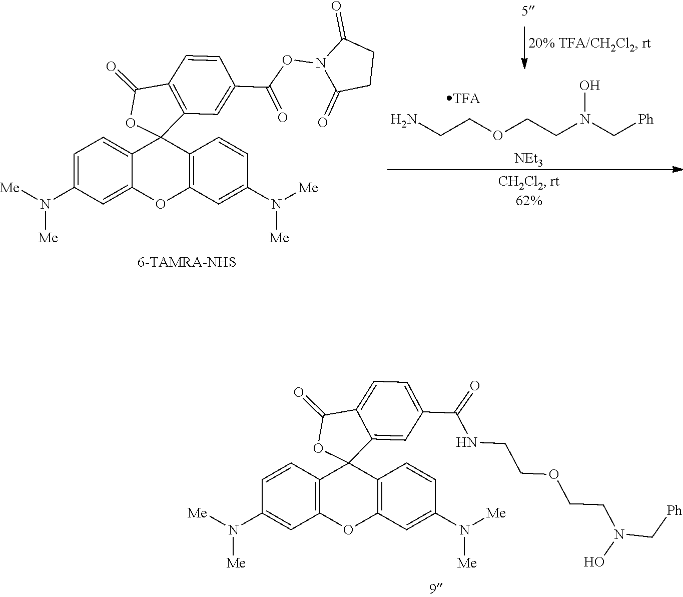 [SMILES: CN(C)c1ccc2c(c1)Oc1cc(N(C)C)ccc1C21OC(=O)c2ccc(C(=O)ON3C(=O)CCC3=O)cc21 ; NCCOCCN(O)Cc1ccccc1 ; CN(C)c1ccc2c(c1)Oc1cc(N(C)C)ccc1C21OC(=O)c2ccc(C(=O)NCCOCCN(O)Cc3ccccc3)cc21 ; "NEt3"]

Trifluoroacetic acid (200 μL) was added to a solution of hydroxylamine 5″ (16.0 mg, 51.5 μmol) in dichloromethane (800 μL). The resulting solution was stirred at room temperature for 45 min and then concentrated under reduced pressure. The resulting residue was dissolved in dichloromethane (1.0 mL). Triethylamine (23.7 μL, 172 μmol) and 6-TAMRA-NHS (18.1 mg, 34.3 μmol) were then sequentially added to the solution. The reaction mixture was stirred at room temperature for 2.5 h, concentrated under reduced pressure, and purified by automated C₁₈ reverse phase column chromatography (30 g C₁₈ silica gel, 25 μm spherical particles, eluent: H₂O+0.1% TFA (5 CV), gradient 0-100% MeCN/H₂O+0.1% TFA (15 CV)) and flash column chromatography on silica gel (eluent: 60% CMA in chloroform) to afford the title compound as a violet solid (13.2 mg, 62%). TLC (70% CMA in chloroform), Rf: 0.30 (UV). ¹H NMR (500 MHz, CD₃OD, 25° C.) δ 8.19 (d, J=8.1 Hz, 1H), 8.11 (dd, J=8.1, 1.8 Hz, 1H), 7.74 (d, J=1.8 Hz, 1H), 7.29-7.26 (m, 2H), 7.26-7.17 (m, 5H), 6.99-6.94 (m, 2H), 6.90 (dd, J=2.6, 1.1 Hz, 2H), 3.77 (s, 2H), 3.68 (t, J=5.5 Hz, 2H), 3.62 (t, J=5.1 Hz, 2H), 3.56 (t, J=5.1 Hz, 2H), 3.26 (s, 12H), 2.86 (t, J=5.4 Hz, 2H). ¹³C NMR (126 MHz, CD₃OD, 25° C.) δ 171.4, 168.7, 161.9, 159.2, 158.9, 142.1, 138.5, 137.3, 134.5, 132.7, 131.7, 130.9, 129.9, 129.9, 129.3, 128.5, 115.3, 115.1, 97.5, 70.5, 69.1, 66.2, 60.6, 41.2, 41.0. FTIR (thin film) cm⁻¹: 3228 (br), 1674 (m), 1595 (s), 1491 (m), 1349 (s), 1185 (s), 1133 (m). HRMS (ESI) (m/z): calc'd for C₃₆H₃₉N₄O₆ [M+H]⁺: 623.2864, found: 623.2849.

### Example 74: Synthesis of tert-Butyl 4-hydroxypiperazine-1-carboxylate (54″)

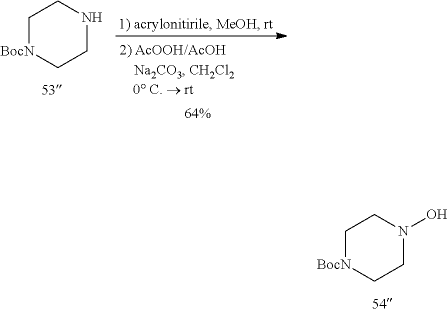 [SMILES: CC(C)(C)OC(=O)N1CCNCC1 ; CC(C)(C)OC(=O)N1CCN(O)CC1]

Acrylonitrile (1.57 mL, 24.0 mmol) was added via syringe to a solution of N-Boc-piperazine (53″, 3.73 g, 20.0 mmol) in methanol (70 mL) at room temperature. After 30 min, the reaction mixture was concentrated and used without further purification. The crude product was dissolved in dichloromethane (200 mL) and solid sodium carbonate (6.36 g, 60.0 mmol) was added in one portion. After the resultant suspension was cooled to 0° C. in an ice-water bath, 39% peracetic acid/acetic acid (3.39 mL, 20.0 mmol) was added via syringe. The ice-water bath was immediately removed and the reaction mixture was allowed to warm to room temperature. After 3 h, the reaction mixture was filtered and methanol (2 mL) was added. The reaction mixture was loaded directly onto a silica gel column. The reaction mixture was purified by flash column chromatography on silica gel (eluent: 4→5% methanol in dichloromethane) to afford the title compound as a white solid (2.59 g, 64%). TLC (5% methanol in dichloromethane), Rf: 0.41 (I₂). ¹H NMR (500 MHz, CDCl₃, 25° C.) δ 3.97 (m, 2H), 3.14 (d, J=10.6 Hz, 2H), 3.06-2.84 (m, 2H), 2.56 (td, J=11.4, 11.0, 3.4 Hz, 2H), 1.44 (s, 9H). ¹³C NMR (126 MHz, CDCl₃, 25° C.) δ 154.7, 80.3, 57.5, 42.2, 28.5. FTIR (thin film) cm⁻¹: 3381 (br), 2974 (w), 2933 (w), 2851 (w), 1670 (m), 1416 (m), 1364 (m), 1249 (s), 1166 (s), 1129 (s), 1036 (m). HRMS (ESI) (m/z): calc'd for C₉H₁₉N₂O₃ [M+H]⁺: 203.1390, found: 203.1388.

### Example 75: Synthesis of 3′,6′-Bis(dimethylamino)-6-(4-hydroxypiperazine-1-carbonyl)-3H-spiro[isobenzofuran-1,9′-xanthen]-3-one (10″)

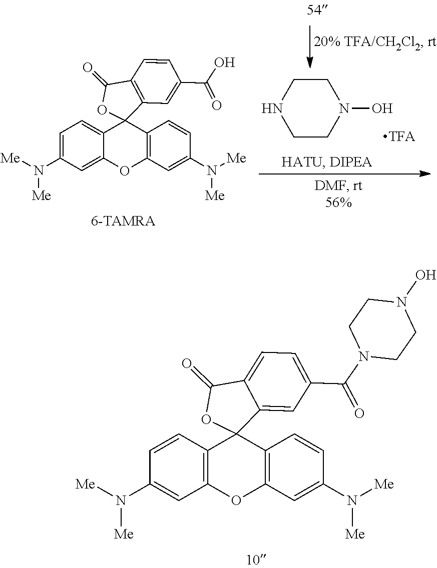

Trifluoroacetic acid (200 μL) was added to a solution of hydroxylamine 54″ (20.7 mg, 102 μmol) in dichloromethane (800 μL). The resulting solution was stirred at room temperature for 45 min then concentrated under reduced pressure. 6-TAMRA (40.0 mg, 92.9 μmol) was added and the mixture was dissolved in N,N-dimethylformamide (1.0 mL). N,N-Diisopropylethylamine (80.9 μL, 465 μmol) and 1-[Bis(dimethylamino)methylene]-1H-1,2,3-triazolo[4,5-b]pyridinium 3-oxide hexafluorophosphate (HATU, 38.9 mg, 102 μmol) were then sequentially added to the solution. The reaction mixture was stirred at room temperature for 2 h, diluted with water, and purified by automated C₁₈ reverse phase column chromatography (30 g C₁₈ silica gel, 25 μm spherical particles, eluent: H₂O+0.1% TFA (5 CV), gradient 0→100% MeCN/H₂O+0.1% TFA (15 CV)) and flash column chromatography on silica gel (eluent: 70% CMA in chloroform) to afford the title compound as a dark violet solid (26.6 mg, 56%). TLC (70% CMA in chloroform), Rf: 0.16 (UV). ¹H NMR (500 MHz, CD₃OD/CDCl₃ [1/1, v/v], 25° C.) δ 8.19 (d, J=7.9 Hz, 1H), 7.66 (dd, J=7.9, 1.7 Hz, 1H), 7.27 (d, J=9.4 Hz, 3H), 6.91 (dd, J=9.4, 2.5 Hz, 2H), 6.80 (s, 2H), 4.45 (s, 1H), 3.78 (s, 1H), 3.31-3.10 (m, 16H), 2.60 (d, J=30.1 Hz, 2H). ¹³C NMR (126 MHz, CD₃OD/CDCl₃ [1/1, v/v], 25° C.) δ 171.1, 170.2, 159.6, 158.4, 157.9, 142.1, 137.0, 134.4, 132.3, 131.0, 128.9, 128.4, 114.4, 114.3, 97.1, 58.2, 57.8, 46.9, 41.4, 41.0. FTIR (thin film) cm⁻¹: 3370 (br), 2930 (w), 1588 (s), 1491 (m), 1409 (m), 1346 (s), 1189 (s). HRMS (ESI) (m/z): calc'd for C₂₉H₃₁N₄O₅ [M+H]⁺: 515.2289, found: 515.2278.

### Example 76: Synthesis of 3-(4-Nitrophenoxy)cyclooct-1-yne (S2″)

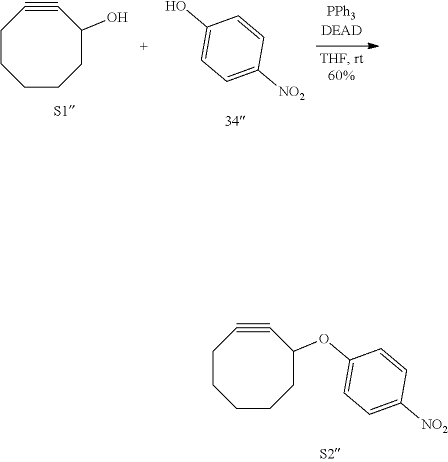 [SMILES: OC1C#CCCCCC1 ; O=[N+]([O-])c1ccc(O)cc1 ; O=[N+]([O-])c1ccc(OC2C#CCCCCC2)cc1]

p-Nitrophenol (67.2 mg, 483 μmol), triphenylphosphine (127 mg, 483 μmol), and diethyl azodicarboxylate (DEAD, 40% in toluene, 220 μL, 483 μmol) were sequentially added to a solution of cyclooct-2-yn-1-ol (Kang, et al., J. Am. Chem. Soc. 143:5616-5621 (2021)) (S1″, 50.0 mg, 403 μmol) in tetrahydrofuran (4.0 mL). The reaction mixture was stirred at room temperature. After 1 h, the solution was concentrated under reduced pressure, and purified by flash column chromatography on silica gel (eluent: 20% dichloromethane in hexanes) to afford the title compound as a white solid (59.7 mg, 60%). TLC (20% dichloromethane in hexanes), Rf: 0.31 (UV, KMnO₄). ¹H NMR (500 MHz, CDCl₃, 25° C.) δ 8.16 (d, J=9.3 Hz, 2H), 6.96 (d, J=9.3 Hz, 2H), 4.82 (tt, J=5.8, 2.1 Hz, 1H), 2.30-2.12 (m, 4H), 1.94-1.83 (m, 3H), 1.78-1.69 (m, 1H), 1.69-1.58 (m, 2H). ¹³C NMR (126 MHz, CDCl₃, 25° C.) δ 163.0, 141.8, 125.9, 115.5, 103.2, 90.4, 71.0, 42.3, 34.3, 29.8, 26.2, 20.8. FTIR (thin film) cm⁻¹: 2930 (m), 2855 (w), 1592 (s), 1495 (s), 1446 (m), 1342 (s), 1249 (S), 1170 (m). HRMS (ESI) (m/z): calc'd for C₁₄H₁₆NO₃ [M+H]⁺: 246.1125, found: 246.1123.

### Example 77: Synthesis of (E)-N,N-Diethyl-3-(4-nitrophenoxy)cyclooct-1-en-1-amine oxide (32″)

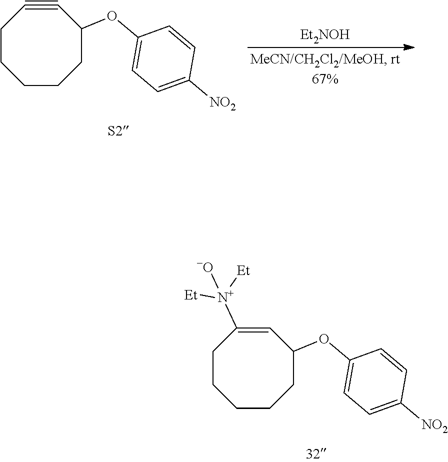

N,N-Diethylhydroxylamine (19.1 μL, 186 μmol) was added to a solution of cyclooctyne S2″ (30.4 mg, 124 μmol) in acetonitrile/dichloromethane/methanol (2/2/1, v/v/v, 3.0 mL). The reaction mixture was stirred at room temperature for 10 min, concentrated under reduced pressure, and purified by flash column chromatography on silica gel (eluent: 30% CMA in chloroform) to afford the title compound as a yellow film (41.5 mg, 67%). TLC (30% CMA in chloroform), Rf 0.25 (UV). ¹H NMR (500 MHz, CD₃OD, 25° C.) δ 8.13 (d, J=9.3 Hz, 2H), 6.95 (d, J=9.3 Hz, 2H), 6.42 (d, J=7.5 Hz, 1H), 5.25 (ddd, J=11.9, 7.5, 4.7 Hz, 1H), 3.67-3.57 (m, 1H), 3.52-3.42 (m, 1H), 3.36-3.30 (m, 1H), 3.31-3.25 (m, 1H), 2.67-2.60 (m, 2H), 2.23-2.13 (m, 1H), 1.94-1.84 (m, 2H), 1.80-1.51 (m, 5H), 1.25 (t, J=7.1 Hz, 3H), 1.07 (t, J=7.1 Hz, 3H). ¹³C NMR (126 MHz, CD₃OD, 25° C.) δ 164.4, 149.6, 143.0, 128.8, 126.9, 117.1, 77.1, 63.7, 62.2, 36.4, 30.9, 27.4, 27.3, 24.5, 9.2, 9.1. FTIR (thin film) cm⁻¹: 3179 (br), 2933 (w), 1588 (m), 1510 (m), 1454 (w), 1338 (s), 1252 (s). HRMS (ESI) (m/z): calc'd for C₁₈H₂₇N₂O₄ [M+H]⁺: 335.1965, found: 335.1962.

### Example 78: Synthesis of Cyclooct-2-yn-1-yl(4-nitrophenyl)sulfane (S4″)

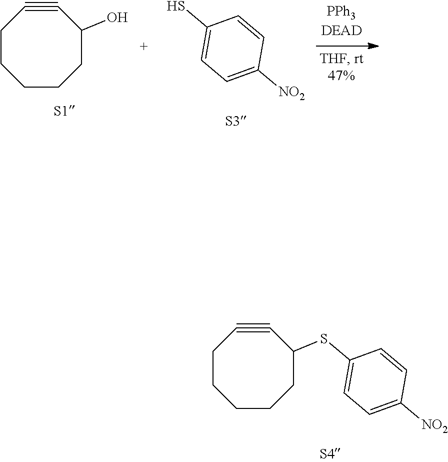 [SMILES: OC1C#CCCCCC1 ; O=[N+]([O-])c1ccc(S)cc1 ; O=[N+]([O-])c1ccc(SC2C#CCCCCC2)cc1]

p-Nitrothiophenol (75.0 mg, 483 μmol) and triphenylphosphine (127 mg, 483 μmol) were sequentially added to a solution of cyclooct-2-yn-1-ol (50.0 mg, 403 μmol) in tetrahydrofuran (4.0 mL) at room temperature. A solution of diethyl azodicarboxylate (DEAD, 40% in toluene, 220 μL, 483 μmol) was then added dropwise via syringe, and the reaction mixture was stirred at room temperature. After 1.5 h, the solution was concentrated under reduced pressure and purified by flash column chromatography on silica gel (eluent: 20% dichloromethane in hexanes) to afford the title compound as a pale yellow solid (49.6 mg, 47%). TLC (15% dichloromethane in hexanes), Rf: 0.14 (UV, KMnO₄). ¹H NMR (500 MHz, CDCl₃, 25° C.) δ 8.11 (d, J=9.0 Hz, 2H), 7.40 (d, J=9.2 Hz, 2H), 4.12 (tq, J=6.9, 2.4 Hz, 1H), 2.33 (ddd, J=13.9, 8.6, 5.6 Hz, 1H), 2.27-2.15 (m, 2H), 2.08-1.98 (m, 1H), 1.94-1.83 (m, 3H), 1.70-1.63 (m, 3H). ¹³C NMR (126 MHz, CDCl₃, 25° C.) δ 146.6, 145.6, 127.7, 124.0, 99.0, 91.7, 41.6, 39.1, 34.5, 29.6, 28.2, 21.0. FTIR (thin film) cm⁻¹: 2930 (m), 2859 (W), 1577 (m), 1506 (s), 1446 (m), 1338 (s), 1182 (s). HRMS (ESI) (m/z): calc'd for C₁₄H₁₆NO₂S [M+H]⁺: 262.0896, found: 262.0895.

### Example 79: Synthesis of (E)-N,N-Diethyl-3-((4-nitrophenyl)thio)cyclooct-1-en-1-amine oxide (38″)

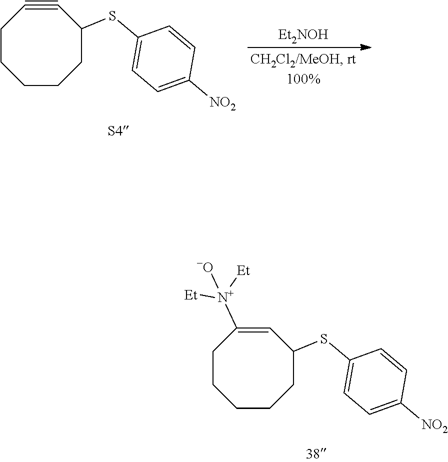

N,N-Diethylhydroxylamine (13.6 mg, 132 μmol) was added to a solution of cyclooctyne S4″ (23.0 mg, 88.0 μmol) in dichloromethane/methanol (1/1, v/v, 1.0 mL). The reaction mixture was stirred at room temperature for 10 min, concentrated under reduced pressure, and purified by flash column chromatography on silica gel (eluent: 25% CMA in chloroform) to afford the title compound as a red film (30.7 mg, 100%). TLC (30% CMA in chloroform), Rf: 0.29 (UV). ¹H NMR (500 MHz, CD₃OD, 25° C.) δ 8.12 (d, J=9.0 Hz, 2H), 7.47 (d, J=9.0 Hz, 2H), 6.45 (d, J=9.3 Hz, 1H), 4.45 (ddd, J=12.7, 9.4, 4.5 Hz, 1H), 3.57 (dd, J=12.7, 7.2 Hz, 1H), 3.46 (dd, J=12.5, 7.2 Hz, 1H), 3.36-3.30 (m, 1H), 3.25 (dd, J=12.7, 7.2 Hz, 1H), 2.71-2.49 (m, 2H), 2.17-2.01 (m, 1H), 1.99-1.74 (m, 4H), 1.65-1.51 (m, 3H), 1.24 (t, J=7.1 Hz, 3H), 0.90 (t, J=7.2 Hz, 3H). ¹³C NMR (126 MHz, CD₃OD, 25° C.) δ 149.7, 147.5, 147.2, 130.1, 129.9, 125.1, 63.7, 62.0, 44.3, 35.7, 30.9, 27.5, 27.3, 26.5, 9.1, 9.0. FTIR (thin film) cm⁻¹: 3183 (br), 2933 (w), 2855 (w), 1577 (m), 1510 (m), 1334 (s), 1096 (w). HRMS (ESI) (m/z): calc'd for C₁₈H₂₇N₂O₃S [M+H]⁺: 351.1737, found: 351.1733.

### Example 80: Synthesis of 3′,6′-Dihydroxy-N-(2-(2-(hydroxy(methyl)amino)ethoxy)ethyl)-3-oxo-3H-spiro[isobenzofuran-1,9′-xanthene]-5-carboxamide (40″)

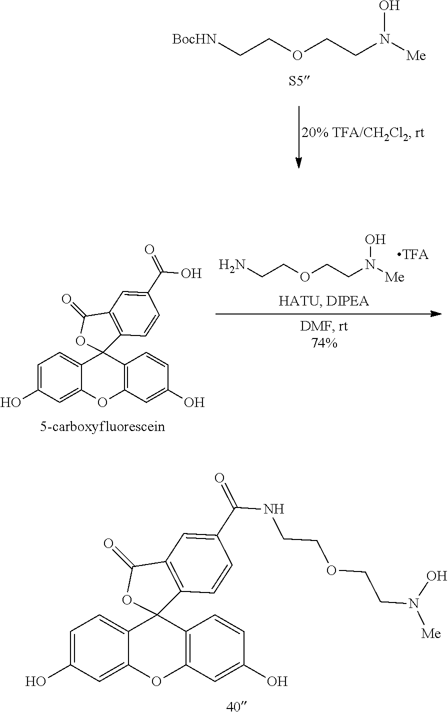 [SMILES: CN(O)CCOCCNC(=O)OC(C)(C)C ; O=C(O)c1ccc2c(c1)C(=O)OC21c2ccc(O)cc2Oc2cc(O)ccc21 ; CN(O)CCOCCN ; CN(O)CCOCCNC(=O)c1ccc2c(c1)C(=O)OC21c2ccc(O)cc2Oc2cc(O)ccc21]

Trifluoroacetic acid (200 μL) was added to a solution of tert-butyl (2-(2-(hydroxy(methyl)amino)ethoxy)ethyl)carbamate (Kang, et al., J. Am. Chem. Soc. 143:5616-5621 (2021)) (S5″, 29.0 mg, 83.3 μmol) in dichloromethane (800 μL). The resulting solution was stirred at room temperature for 30 min then concentrated under reduced pressure. Separately, N,N-diisopropylethylamine (72.5 μL, 417 μmol) was added to a solution of 5-carboxyfluorescein (34.5 mg, 91.6 μmol) in N,N-dimethylformamide (500 μL). The resulting solution was transferred via cannula to the vial containing the hydroxylamine intermediate. An additional portion of N,N-dimethylformamide (500 μL) was used to complete the transfer of the 5-carboxyfluorescein solution. HATU (34.8 mg, 91.6 μmol) was then added to the solution. The reaction mixture was stirred at room temperature for 1 h, concentrated under reduced pressure, and purified by flash column chromatography on silica gel (eluent: 10% methanol in dichloromethane) and automated C₁₈ reverse phase column chromatography (30 g C₁₈ silica gel, 25 μm spherical particles, eluent: H₂O+0.1% TFA (5 CV), gradient 0→100% MeCN/H₂O+0.1% TFA (15 CV)) to afford the title compound as a yellow oil (30.4 mg, 74%). TLC (15% methanol in dichloromethane), Rf: 0.27 (UV). ¹H NMR (500 MHz, CD₃OD, 25° C.) δ 8.52 (d, J=2.0 Hz, 1H), 8.24 (dd, J=8.0, 1.8 Hz, 1H), 7.36 (d, J=8.1 Hz, 1H), 6.84 (d, J=2.4 Hz, 2H), 6.77 (d, J=8.9 Hz, 2H), 6.67 (dd, J=8.9, 2.4 Hz, 2H), 3.93 (ddd, J=11.6, 8.7, 3.0 Hz, 1H), 3.81 (dt, J=11.4, 3.9 Hz, 1H), 3.78-3.60 (m, 5H), 3.55 (ddd, J=13.6, 4.6, 3.0 Hz, 1H), 3.23 (s, 3H). ¹³C NMR (126 MHz, CD₃OD, 25° C.) δ 168.4, 167.3, 162.5, 160.1, 154.1, 151.2, 136.4, 133.5, 129.5, 128.1, 125.6, 124.9, 114.0, 110.9, 102.2, 69.7, 63.2, 59.8, 45.6, 39.4. FTIR (thin film) cm⁻¹: 3289 (br), 1681 (m), 1640 (m), 1592 (s), 1465 (m), 1208 (s), 1182 (s), 1133 (s). HRMS (ESI) (m/z): calc'd for C₂₆H₂₅N₂O₈[M+H]⁺: 493.1605, found: 493.1596.

### Example 81: Synthesis of N-((Cyclooct-2-yn-1-yloxy)carbonyl)-N-methylglycine (S6″)

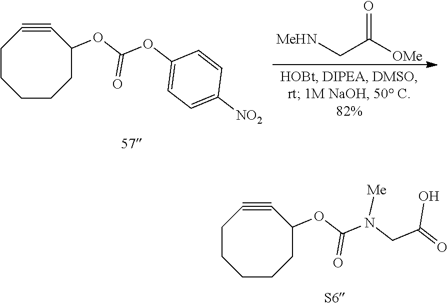 [SMILES: O=C(Oc1ccc([N+](=O)[O-])cc1)OC1C#CCCCCC1 ; CNCC(=O)OC ; CN(CC(=O)O)C(=O)OC1C#CCCCCC1]

Dimethylsulfoxide (674 μL) was added to a vial charged with 4-nitrophenyl carbonate 57″ (Plass, et al., Angew. Chem., Int. Ed. 50:3878-3881 (2011)) (19.5 mg, 67.4 μmol), N-methylglycine methyl ester hydrochloride (18.8 mg, 135 μmol), and 1-hydroxybenzotriazole hydrate (20% H₂O w/w, 11.4 mg, 67.4 μmol) at room temperature. N,N-Diisopropylethylamine (35.2 μL, 202 μmol) was then added via syringe. After 2.5 h, an aqueous solution of sodium hydroxide (1 M, 700 μL) was added to the reaction mixture, and the solution was heated to 50° C. After 2 h, the resulting mixture was cooled to room temperature, diluted with ethyl acetate (15 mL), and acidified with an aqueous solution of hydrochloric acid (1 M, 15 mL). The organic layer was washed with brine (10 mL), dried over anhydrous sodium sulfate, filtered, and concentrated under reduced pressure. The resulting crude residue was purified by flash column chromatography on silica gel (eluent: 50% ethyl acetate in hexanes then 5% methanol in dichloromethane) to afford the title compound as a colorless film (13.2 mg, 82%). TLC (7.5% methanol in dichloromethane), Rf: 0.16 (I₂). ¹H NMR (500 MHz, DMSO-d₆, 1:0.91 mixture of rotamers, 25° C.) δ 12.73 (br s, 1H), 5.26-4.98 (m, 1H), 4.02-3.78 (m, 2H), 2.84 (two s, 3H), 2.32-2.19 (m, 1H), 2.19-1.98 (m, 2H), 1.97-1.78 (m, 3H), 1.77-1.39 (m, 4H). ¹H NMR (500 MHz, DMSO-d₆, 75° C.) δ 5.26-5.01 (m, 1H), 4.05-3.79 (m, 2H), 2.86 (s, 3H), 2.31-2.20 (m, 1H), 2.16 (dtd, J=16.9, 6.2, 2.5 Hz, 1H), 2.13-2.02 (m, 1H), 2.00-1.79 (m, 3H), 1.78-1.67 (m, 1H), 1.69-1.44 (m, 3H). ¹³C NMR (126 MHz, DMSO-d₆, 1:0.91 mixture of rotamers, 25° C.) δ 170.8, 155.1, 154.8, 101.3, 91.5, 91.4, 67.0, 66.9, 50.0, 49.8, 41.5, 41.5, 35.5, 34.8, 33.8, 33.8, 29.2, 29.1, 25.7, 25.6, 20.0, 20.0. FTIR (thin film) cm⁻¹: 2930 (m), 2855 (w), 1700 (s), 1484 (m), 1450 (m), 1401 (m), 1301 (w), 1342 (w), 1223 (m), 1152 (s). HRMS (ESI) (m/z): calc'd for C₁₂H₁₈NO₄ [M+H]⁺: 240.1230, found: 240.1229.

### Example 82: Synthesis of Perfluorophenyl N-((cyclooct-2-yn-1-yloxy)carbonyl)-N-methylglycinate (43″)

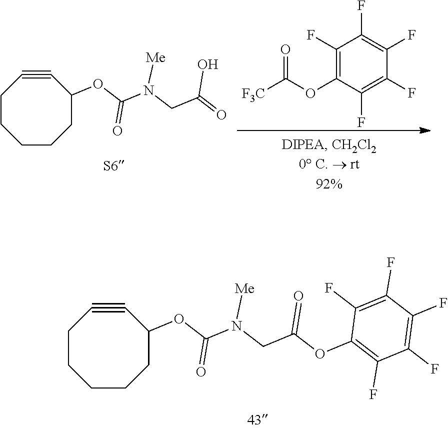

N,N-Diisopropylethylamine (11.0 μL, 61.9 μmol) and pentafluorophenyl trifluoroacetate (5.30 μL, 31.0 μmol) were sequentially added to a solution of acid S6″ (3.70 mg, 15.5 μmol) in dichloromethane (500 μL) at room temperature. After 1 h, the resulting mixture was diluted with hexanes and directly purified by flash column chromatography on silica gel (eluent: 10% ethyl acetate in hexanes) to afford the title compound as a colorless oil (14.2 mg, 92%). TLC (20% ethyl acetate in hexane), Rf: 0.47 (UV, I₂). ¹H NMR (500 MHz, CDCl₃, −1:1 mixture of rotamers, 25° C.) δ 5.37-5.08 (m, 1H), 4.49 (dd, J=18.3, 4.5 Hz, 1H), 4.28 (d, J=18.1 Hz, 0.5H), 4.15 (d, J=18.4 Hz, 0.5H), 3.04 (s, 1.5H), 3.03 (s, 1.5H), 2.33-2.22 (m, 1H), 2.21-2.08 (m, 2H), 2.08-1.94 (m, 1H), 1.94-1.69 (m, 3H), 1.69-1.57 (m, 2H), 1.58-1.44 (m, 1H). ¹HNMR (500 MHz, CDCl₃, −1:1 mixture of rotamers, 50° C.) δ 5.31 (m, 1H), 4.47 (d, J=18.3 Hz, 1H), 4.28 (d, J=17.8 Hz, 0.5H), 4.16 (d, J=18.4 Hz, 0.5H), 3.04 (s, 3H), 2.31-2.22 (m, 1H), 2.16 (dd, J=16.9, 6.4 Hz, 2H), 2.08-1.96 (m, 1H), 1.96-1.82 (m, 2H), 1.83-1.70 (m, 1H), 1.71-1.60 (m, 2H), 1.59-1.49 (m, 1H). ¹³C NMR (126 MHz, CDCl₃, ˜1:1 mixture of rotamers, 25° C.) δ 166.0, 156.1, 155.2, 102.1, 91.1, 90.9, 68.7, 68.7, 50.2, 50.2, 42.0, 41.9, 36.2, 35.4, 34.4, 34.4, 29.8, 29.8, 26.4, 26.3, 20.9, 20.9. ¹⁹F NMR (470 MHz, CDCl₃, −1:1 mixture of rotamers, 25° C.) 6-152.0-−152.3 (m), −152.3-−152.6 (m), −157.2 (t, J=21.6 Hz), −157.4 (t, J=21.7 Hz), −161.72-−161.91 (m), −161.91-−162.09 (m). FTIR (thin film) cm⁻¹: 2930 (w), 2855 (w), 1804 (w), 1711 (m), 1521 (s), 1454 (w), 1398 (w), 1234 (w), 1156 (w), 1107 (m), 999 (m). HRMS (ESI) (m/z): calc'd for C₁₈H₁₇F₅NO₄ [M+H]⁺: 406.1072, found: 406.1067.

### Example 83: Synthesis of 4-(Cyclooct-2-yn-1-yloxy)-4-oxobutanoic acid (S7″)

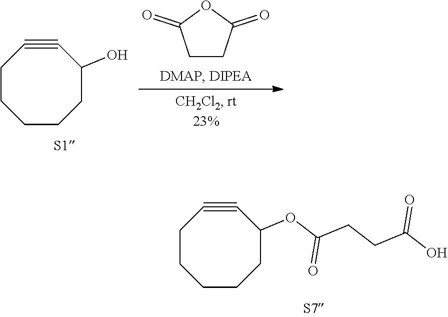 [SMILES: OC1C#CCCCCC1 ; O=C1CCC(=O)O1 ; O=C(O)CCC(=O)OC1C#CCCCCC1]

Succinic anhydride (32.0 mg, 320 μmol), N,N-dimethylaminopyridine (DMAP, 2.60 mg, 21.3 μmol), and N,N-diisopropylethylamine (55.8 μL, 320 μmol) were added sequentially to a solution of cyclooct-2-yn-1-ol (26.5 mg, 213 μmol) in dichloromethane (2.10 mL) at room temperature. After 2.5 h, the reaction mixture was directly purified by flash column chromatography on silica gel (eluent: 2.5% methanol in dichloromethane) to afford the title compound as a clear foam (10.8 mg, 23%). TLC (2.5% methanol in dichloromethane), Rf: 0.14 (I₂). ¹H NMR (500 MHz, CD₃OD, 25° C.) δ 5.38-5.21 (m, 1H), 2.57 (s, 4H), 2.26 (dtd, J=16.8, 6.4, 1.8 Hz, 1H), 2.17 (dtd, J=16.9, 6.3, 3.1 Hz, 1H), 2.15-2.08 (m, 1H), 2.01 (dddd, J=13.9, 8.9, 6.2, 1.1 Hz, 1H), 1.96-1.86 (m, 2H), 1.85-1.76 (m, 1H), 1.76-1.68 (m, 1H), 1.68-1.62 (m, 1H), 1.62-1.52 (m, 1H). ¹³C NMR (126 MHz, CD₃OD, 25° C.) δ 176.1, 173.5, 102.6, 91.8, 68.0, 42.7, 35.4, 30.9, 30.3, 29.9, 27.4, 21.3. FTIR (thin film) cm⁻¹: 3414 (br), 2930 (m), 2855 (w), 1737 (s), 1439 (w), 1342 (w), 1163 (m). HRMS (ESI) (m/z): calc'd for C₁₂H₁₅O₄[M−H]⁻: 223.0976, found: 223.0973.

### Example 84: Synthesis of Cyclooct-2-yn-1-yl (perfluorophenyl) succinate (44″)

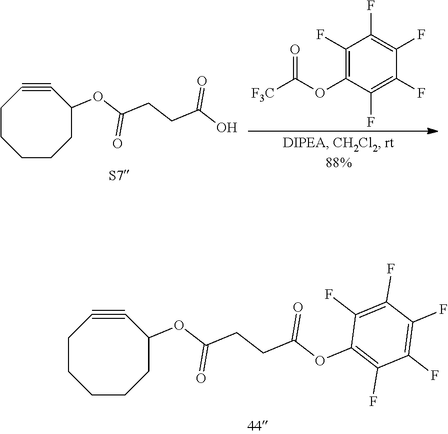 [SMILES: O=C(O)CCC(=O)OC1C#CCCCCC1 ; O=C(Oc1c(F)c(F)c(F)c(F)c1F)C(F)(F)F ; O=C(CCC(=O)OC1C#CCCCCC1)Oc1c(F)c(F)c(F)c(F)c1F]

N,N-Diisopropylethylamine (31.8 μL, 182 μmol) and pentafluorophenyl trifluoroacetate (15.7 μL, 91.2 μmol) were sequentially added via syringe to a solution of acid S7″ (15.7 mg, 60.8 μmol) in dichloromethane (1.00 mL) at room temperature. After 1 h, the resulting mixture was diluted with hexanes (1 mL) and directly purified by flash column chromatography on silica gel (eluent: 30% dichloromethane in hexanes) to afford the title compound as a colorless oil (22.6 mg, 88%). TLC (50% dichloromethane in hexane), Rf: 0.33 (UV, I₂). ¹H NMR (500 MHz, CDCl₃, 25° C.) δ 5.48-5.21 (m, 1H), 3.05-2.91 (m, 2H), 2.75 (t, J=6.9 Hz, 2H), 2.26 (dtd, J=16.9, 6.4, 1.9 Hz, 1H), 2.22-2.08 (m, 2H), 2.06-1.95 (m, 1H), 1.96-1.82 (m, 2H), 1.82-1.73 (m, 1H), 1.73-1.58 (m, 2H), 1.57-1.46 (m, 1H). ¹³C NMR (126 MHz, CDCl₃, 25° C.) δ 170.7, 168.5, 102.6, 90.4, 67.5, 41.6, 34.3, 29.8, 29.2, 28.6, 26.3, 20.9. ¹⁹F NMR (470 MHz, CDCl₃) δ −152.2-−152.7 (m), −157.9 (t, J=21.6 Hz), −161.8-−162.9 (m). FTIR (thin film) cm⁻¹: 2933 (w), 2855 (w), 1789 (m), 1741 (m), 1521 (s), 1454 (w), 1181 (w), 1107 (m), 999 (m). HRMS (ESI) (m/z): calc'd for C₁₈H₁₆F₅O₄ [M+H]⁺: 391.0963, found: 391.0958.

### Example 85: Synthesis of 1-((4-(Cyclooct-2-yn-1-yloxy)benzoyl)oxy)-2,5-dioxopyrrolidine-3-sulfonic acid (45″)

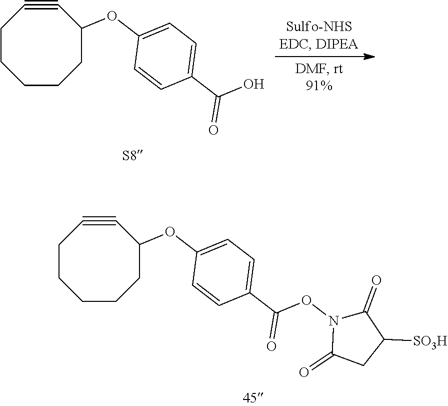 [SMILES: O=C(O)c1ccc(OC2C#CCCCCC2)cc1 ; O=C(ON1C(=O)CC(S(=O)(=O)O)C1=O)c1ccc(OC2C#CCCCCC2)cc1]

N,N-dimethylformamide (500 μL) and N,N-diisopropylethylamine (32.0 μL, 184 μmol) were sequentially added via syringe to a vial charged with benzoic acid S8″ (Hagendorn, et al., Eur. J. Org. Chem. 2014:1280-1286 (2014)) (7.50 mg, 30.7 μmol) and N-hydroxysulfosuccinimide sodium salt (26.7 mg, 123 μmol) at room temperature. 1-Ethyl-3-(3-dimethylaminopropyl)carbodiimide hydrochloride (11.8 mg, 61.4 μmol) was then added to the reaction mixture. After 36 h, the solution was cooled to 0° C., and acetic acid (17.6 uL, 307 μmol) was added. The solution was then diluted with water and purified by automated C₁s reverse phase column chromatography (30 g C₁s silica gel, 25 μm spherical particles, eluent: H₂O+0.1% TFA (5 CV), gradient 0→100% MeCN/H₂O+0.1% TFA (10 CV)). Fractions containing the desired product were collected and concentrated under reduced pressure to a volume of −1 mL. This solution was purified again by automated C₁s reverse phase column chromatography (30 g C₁s silica gel, 25 μm spherical particles, eluent: H₂O (5 CV), gradient 0→100% MeCN/H₂O (10 CV)) to afford the title compound as a colorless film (11.8 mg, 91%). ¹H NMR (500 MHz, CD₃OD, 25° C.) δ 8.12-7.96 (m, 2H), 7.14-7.00 (m, 2H), 4.98 (td, J=5.4, 2.4 Hz, 1H), 4.29 (dd, J=8.4, 3.5 Hz, 1H), 3.35-3.23 (m, 1H), 3.23-3.14 (m, 1H), 2.36-2.14 (m, 4H), 1.99-1.85 (m, 3H), 1.84-1.74 (m, 1H), 1.74-1.62 (m, 2H). ¹³C NMR (126 MHz, CD₃OD, 25° C.) δ 170.0, 166.8, 164.9, 162.8, 133.6, 118.7, 117.0, 103.3, 91.9, 71.7, 58.1, 43.4, 35.4, 31.7, 31.0, 27.3, 21.3. FTIR (thin film) cm⁻¹: 3474 (br), 3209 (br), 2930 (w), 2855 (w), 1767 (m), 1737 (s), 1603 (s), 1357 (w), 1245 (s), 1174 (m), 1081 (m), 1044 (m), 988 (m). HRMS (ESI) (m/z): calc'd for C₁₉H₁₈NO₈S [M−H]⁻: 420.0759, found: 420.0762.

### Example 86: Synthesis of Methyl 2-(cyclooct-2-yn-1-yloxy)acetate (S9″)

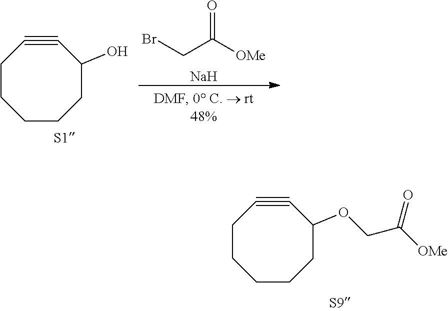

Sodium hydride (60% w/w dispersion in mineral oil, 16.0 mg, 400.0 μmol) was added to a solution of cyclooct-2-yn-1-ol (24.8 mg, 200 μmol) in N,N-dimethylformamide (500 μL) at 0° C. in an ice-water bath. After 5 min, methyl bromoacetate (38.0 μL, 400 μmol) was added to the reaction mixture via syringe, the ice-water bath was removed, and the resulting mixture was allowed to warm to room temperature. After 1.5 h, the reaction mixture was quenched with saturated aqueous ammonium chloride solution (4 mL) and extracted with ethyl acetate (3×4 mL). The combined organic layers were washed with brine (2×10 mL), dried over anhydrous magnesium sulfate, filtered, and concentrated under reduced pressure. The resulting crude residue was purified by flash column chromatography on silica gel (eluent: 9% ethyl acetate in hexanes) to afford the title compound as a colorless oil (18.9 mg, 48%). TLC (20% ethyl acetate in hexane), Rf: 0.46 (I₂). ¹H NMR (500 MHz, CDCl₃, 25° C.) δ 4.37 (ddt, J=7.2, 5.3, 2.1 Hz, 1H), 4.19 (d, J=16.4 Hz, 1H), 4.06 (d, J=16.3 Hz, 1H), 3.72 (s, 3H), 2.23 (dddd, J=16.8, 7.9, 6.1, 1.9 Hz, 1H), 2.19-2.10 (m, 2H), 2.02 (dddd, J=13.7, 9.3, 6.8, 1.1 Hz, 1H), 1.97-1.86 (m, 1H), 1.86-1.75 (m, 2H), 1.70-1.56 (m, 2H), 1.49-1.37 (m, 1H). ¹³C NMR (126 MHz, CDCl₃, 25° C.) δ 171.0, 101.6, 91.7, 73.1, 66.4, 52.0, 42.4, 34.5, 29.9, 26.5, 20.9. FTIR (thin film) cm⁻¹: 2930 (s), 2855 (w), 2210 (w), 1756 (s), 1439 (w), 1208 (m), 1126 (s). HRMS (ESI) (m/z): calc'd for C₁₁H₁₇O₃[M+H]⁺: 197.1172, found: 197.1171.

### Example 87: Synthesis of Perfluorophenyl 2-(cyclooct-2-yn-1-yloxy)acetate (46″)

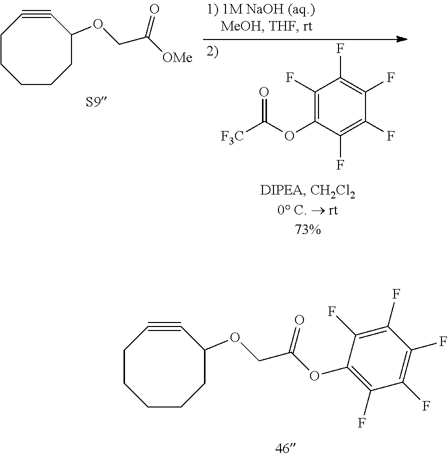 [SMILES: COC(=O)COC1C#CCCCCC1 ; O=C(Oc1c(F)c(F)c(F)c(F)c1F)C(F)(F)F ; O=C(COC1C#CCCCCC1)Oc1c(F)c(F)c(F)c(F)c1F]

An aqueous solution of sodium hydroxide (1 M, 300 μL) was added to a solution of methyl ester S9″ (3.7 mg, 18.8 μmol) in tetrahydrofuran (300 μL) and methanol (300 μL) at room temperature. After 3 h, the solution was acidified with an aqueous solution of hydrochloric acid (1 M, 1.00 mL) and extracted with ethyl acetate (3×2 mL). The combined organic layers were dried over anhydrous magnesium sulfate, filtered, and concentrated under reduced pressure. The resulting crude residue was dissolved in dichloromethane (500 μL), and N,N-diisopropylethylamine (13.0 μL, 75.2 μmol) and pentafluorophenyl trifluoroacetate (6.50 μL, 37.7 μmol) were sequentially added via syringe at room temperature. After 1.5 h, the resulting mixture was diluted with hexanes and directly purified by flash column chromatography on silica gel (eluent: 5% ethyl acetate in hexanes) to afford the title compound as a colorless oil (4.80 mg, 73%). TLC (20% dichloromethane in hexane), Rf: 0.31 (UV, I₂). ¹H NMR (500 MHz, CDCl₃, 25° C.) δ 4.54 (d, J=17.3 Hz, 1H), 4.49-4.39 (m, 1H), 4.43 (d, J=17.3 Hz, 1H), 2.27 (dddd, J=16.8, 7.7, 6.1, 1.8 Hz, 1H), 2.22-2.12 (m, 2H), 2.06 (dddd, J=13.7, 9.1, 6.5, 1.1 Hz, 1H), 1.98-1.89 (m, 1H), 1.89-1.75 (m, 2H), 1.71-1.60 (m, 2H), 1.51-1.44 (m, 1H). ¹³C NMR (126 MHz, CDCl₃, 25° C.) δ 166.7, 102.6, 91.0, 73.6, 65.5, 42.5, 34.5, 29.8, 26.3, 20.9. ¹⁹F NMR (470 MHz, CDCl₃, 25° C.) 6-151.5-−153.1 (m), −157.5 (t, J=21.6 Hz), −160.7-−162.4 (m). FTIR (thin film) cm⁻¹: 2930 (w), 2855 (w), 1808 (w), 1521 (s), 1450 (w), 1144 (w), 1096 (m), 999 (m). HRMS (ESI) (m/z): calc'd for C₁₆H₁₄F₅O₃ [M+H]⁺: 349.0858, found: 349.0856.

### Example 88: Synthesis of 6-(2,5-Dioxo-2,5-dihydro-1H-pyrrol-1-yl)-N-(2-(2-(hydroxy(methyl)amino)ethoxy)ethyl)hexanamide (S10″)

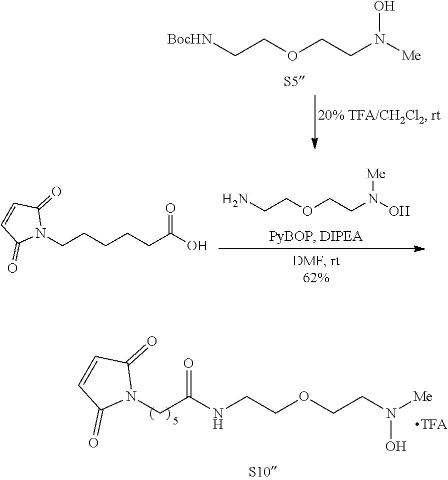

Trifluoroacetic acid (100 μL) was added to a solution of hydroxylamine S5″ (35.1 mg, 150 μmol) in dichloromethane (400 μL). The resulting solution was stirred at room temperature for 2 h then concentrated under reduced pressure. The resulting crude residue was dissolved in N,N-dimethylformamide (1.00 mL). N,N-Diisopropylethylamine (105 μL, 600 μmol), 6-maleimidohexanoic acid (21.1 mg, 100 μmol), and benzotriazol-1-yloxytripyrrolidinophosphonium hexafluorophosphate (PyBOP, 62.4 mg, 120 μmol) were then sequentially added to the reaction mixture. After 50 min, the resulting mixture was diluted with water and purified by automated C₁₈ reverse phase column chromatography (30 g C₁₈ silica gel, 25 μm spherical particles, eluent: H₂O+0.1% TFA (5 CV), gradient 0→100% MeCN/H₂O+0.1% TFA (10 CV)). Fractions containing the desired product were collected and concentrated under reduced pressure to afford the title compound as a colorless oil (27.5 mg, 62%). ¹H NMR (500 MHz, CD₃OD, 25° C.) δ 6.80 (s, 2H), 3.87 (ddd, J=11.7, 8.8, 3.0 Hz, 1H), 3.76 (ddd, J=11.4, 4.5, 3.3 Hz, 1H), 3.62 (ddd, J=13.6, 8.8, 3.4 Hz, 1H), 3.46-3.39 (m, 2H), 3.53 (ddd, J=13.7, 4.6, 3.1 Hz, 1H), 3.49 (t, J=7.1 Hz, 2H), 3.46-3.39 (m, 1H), 3.35 (dt, J=14.2, 5.3 Hz, 1H), 3.22 (s, 3H), 2.20 (t, J=7.4 Hz, 2H), 1.72-1.49 (m, 4H), 1.34-1.25 (m, 2H). ¹³C NMR (126 MHz, CD₃OD, 25° C.) δ 176.7, 172.7, 135.5, 71.4, 64.7, 61.3, 47.2, 40.1, 38.5, 37.0, 29.4, 27.4, 26.5. ¹⁹F NMR (470 MHz, CD₃OD) δ −77.3. FTIR (thin film) cm⁻¹: 3317 (br), 3097 (w), 2937 (w), 2870 (w), 1703 (s), 1550 (w), 1442 (w), 1409 (w), 1200 (s), 1137 (s). HRMS (ESI) (m/z): calc'd for C₁₅H₂₆N₃O₅ [M+H]⁺: 328.1867, found: 328.1864.

### Example 89: Synthesis of N-(2-(2-(Benzyl(hydroxy)amino)ethoxy)ethyl)-6-(2,5-dioxo-2,5-dihydro-1H-pyrrol-1-yl)hexanamide (S11″)

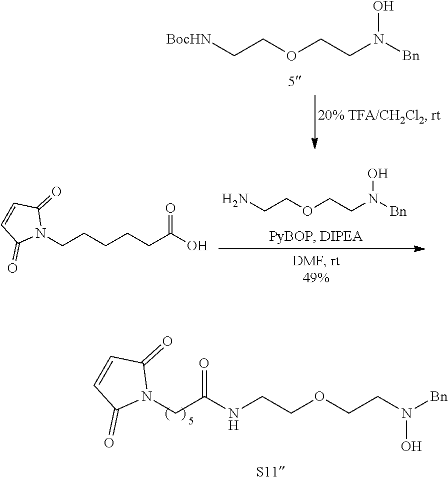

Trifluoroacetic acid (150 μL) was added to a solution of hydroxylamine 5″ (67.5 mg, 218 μmol) in dichloromethane (600 μL). The resulting solution was stirred at room temperature for 2 h and then concentrated under reduced pressure. The resulting crude residue was dissolved in N,N-dimethylformamide (1.40 mL). N,N-Diisopropylethylamine (150 μL, 870 μmol), 6-maleimidohexanoic acid (30.6 mg, 145 μmol), and PyBOP (90.5 mg, 174 μmol) were then sequentially added to the reaction mixture. After 1 h, the resulting mixture was diluted with water and purified by automated C₁₈ reverse phase column chromatography (30 g C₁₈ silica gel, 25 μm spherical particles, eluent: H₂O+0.1% TFA (5 CV), gradient 0→100% MeCN/H₂O+0.1% TFA (10 CV)). Fractions containing the desired product were collected and concentrated under reduced pressure. The resulting residue was then purified by flash column chromatography on silica gel (eluent: 2.5% methanol in dichloromethane) to afford the title compound as a colorless oil (28.8 mg, 49%). TLC (2.5% methanol in dichloromethane), Rf: 0.21 (UV, I₂). ¹H NMR (500 MHz, CD₃OD, 25° C.) δ 7.50-7.43 (m, 2H), 7.43-7.35 (m, 3H), 6.79 (s, 2H), 4.26 (s, 2H), 3.77 (t, J=5.2 Hz, 2H), 3.54 (t, J=5.4 Hz, 2H), 3.47 (t, J=7.1 Hz, 2H), 3.36 (t, J=5.7 Hz, 2H), 3.28-3.20 (m, 2H), 2.17 (t, J=7.5 Hz, 2H), 1.73-1.42 (m, 4H), 1.39-1.16 (m, 2H). ¹³C NMR (126 MHz, CD₃OD, 25° C.) δ 176.4, 172.7, 135.5, 132.2, 130.1, 129.8, 71.1, 66.7, 65.3, 59.8, 40.2, 38.5, 37.0, 29.4, 27.5, 27.4, 26.5. FTIR (thin film) cm⁻¹: 3332 (br), 3094 (w), 2937 (w), 2870 (w), 1703 (s), 1651 (m), 1543 (w), 1409 (m), 1192 (s), 1137 (s). HRMS (ESI) (m/z): calc'd for C₂₁H₃₀N₃O₅ [M+H]⁺: 404.2180, found: 404.2172.

### Example 90: Synthesis of 1-(6-(4-Hydroxypiperazin-1-yl)-6-oxohexyl)-1H-pyrrole-2,5-dione (56″)

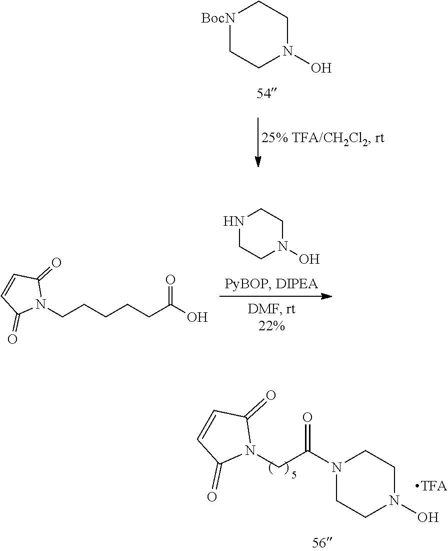

Trifluoroacetic acid (200 μL) was added to a solution of hydroxylamine 54″ (60.6 mg, 300 μmol) in dichloromethane (600 μL). The resulting solution was stirred at room temperature for 1.5 h then concentrated under reduced pressure. The resulting crude residue was dissolved in N,N-dimethylformamide (2.00 mL). N,N-Diisopropylethylamine (200 μL, 1.20 mmol), 6-maleimidohexanoic acid (42.2 mg, 200 μmol), and PyBOP (124.8 mg, 240 μmol) were then sequentially added to the reaction mixture. After 1.5 h, the resulting mixture was diluted with water and purified by automated C₁₈ reverse phase column chromatography (30 g C₁₈ silica gel, 25 μm spherical particles, eluent: H₂O+0.1% TFA (5 CV), gradient 0→100% MeCN/H₂O+0.1% TFA (10 CV)). Fractions containing the desired product were collected and concentrated under reduced pressure to afford the title compound as a yellow oil (17.8 mg, 22%). ¹H NMR (500 MHz, CD₃OD, 25° C.) δ 6.80 (s, 2H), 4.27-4.13 (m, 1H), 4.13-4.01 (m, 1H), 3.79-3.57 (m, 4H), 3.50 (t, J=7.0 Hz, 2H), 3.44-3.22 (m, 2H), 2.44 (t, J=7.5 Hz, 2H), 1.72-1.54 (m, 4H), 1.43-1.22 (m, 2H). ¹³C NMR (126 MHz, CD₃OD, 25° C.) δ 174.1, 172.8, 135.5, 56.6, 56.5, 42.0, 38.4, 38.1, 33.5, 29.4, 27.4, 25.7. ¹⁹F NMR (471 MHz, CD₃OD, 25° C.) 6-77.4. FTIR (thin film) cm⁻¹: 3422 (br), 2945 (w), 2866 (w), 1703 (s), 1442 (m), 1413 (m), 1196 (s), 1141 (s). HRMS (ESI) (m/z): calc'd for C₁₄H₂₂N₃O₄ [M+H]⁺: 296.1605, found: 296.1602.

### Example 91: Synthesis of Monomethyl Auristatin E Cyclooctynyl Carbamate (MMAE-COT 58″

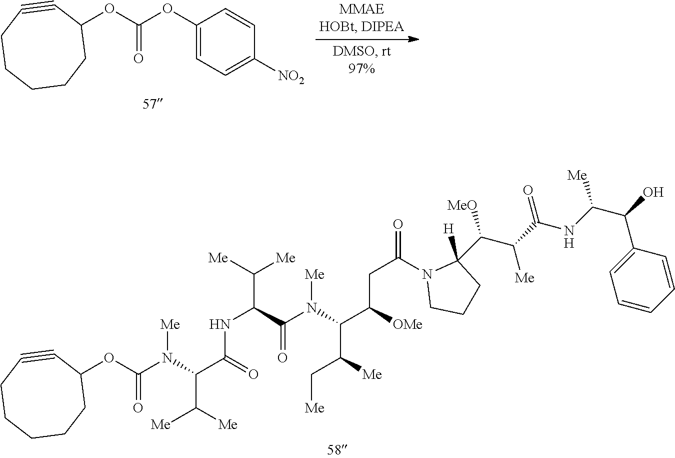 [SMILES: O=C(Oc1ccc([N+](=O)[O-])cc1)OC1C#CCCCCC1 ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC1C#CCCCCC1)C(C)C]

N,N-Diisopropylethylamine (2.4 μL, 19.4 μmol) was added to a solution of monomethyl auristatin E (MMAE, 9.30 mg, 13.0 μmol), cyclooctynyl p-nitrophenylcarbonate 57″ (5.6 mg, 19.4 μmol), and 1-hydroxybenzotriazole hydrate (HOBt, 2.2 mg, 13.0 μmol; 20% H₂O w/w) in DMSO (400 μL). The reaction mixture was stirred at room temperature for 7 h, diluted with H₂O, and purified by automated C₁₈ reverse phase column chromatography (30 g C₁₈ silica gel, 25 μm spherical particles, eluent: H₂O+0.1% TFA (5 CV), gradient 0→100% MeCN/H₂O+0.1% TFA (10 CV)). Fractions containing the desired product were collected and concentrated under reduced pressure to afford the title compound as a clear film (10.9 mg, 97%). ¹H NMR (500 MHz, DMSO-d₆, 25° C.) δ 7.31 (d, J=7.6 Hz, 2H), 7.26 (t, J=7.6 Hz, 2H), 7.17 (t, J=7.2 Hz, 1H), 5.21-5.10 (m, 1H), 4.46 (dd, J=28.3, 6.3 Hz, 1H), 4.26-4.11 (m, 1H), 4.05-3.91 (m, 2H), 3.63-3.52 (m, 1H), 3.32-3.10 (m, 10H), 3.01-2.95 (m, 1H), 2.83 (d, J=17.7 Hz, 3H), 2.50 (s, 2H), 2.40 (d, J=15.0 Hz, 1H), 2.31-2.20 (m, 1H), 2.18-2.02 (m, 4H), 1.87-1.70 (m, 6H), 1.63-1.45 (m, 5H), 1.35-1.18 (m, 2H), 1.10-0.94 (m, 7H), 0.91-0.74 (m, 20H). ¹³C NMR (126 MHz, DMSO-d₆, 25° C.) δ 172.4, 172.3, 172.2, 169.7, 168.7, 158.4, 158.1, 155.3, 155.0, 143.6, 127.8, 127.7, 126.7, 126.6, 126.5, 126.4, 101.3, 101.1, 100.9, 100.5, 91.9, 91.6, 91.5, 85.4, 81.6, 77.7, 77.0, 74.7, 67.1, 67.0, 67.0, 63.3, 63.2, 60.9, 60.3, 58.7, 58.2, 57.1, 57.1, 55.0, 54.1, 49.7, 49.6, 49.1, 47.2, 46.2, 43.7, 43.2, 41.6, 41.6, 41.1, 41.0, 37.2, 35.1, 33.9, 32.0, 31.8, 31.5, 30.0, 29.6, 29.2, 29.2, 29.0, 27.0, 26.6, 26.0, 25.9, 25.8, 25.7, 25.3, 24.4, 24.3, 23.1, 20.0, 20.0, 19.0, 18.9, 18.6, 18.4, 18.2, 15.8, 15.6, 15.4, 15.3, 15.2, 15.2, 15.0, 10.4, 10.3. FTIR (thin film) cm⁻¹: 3317 (br), 2933(w), 1782 (w), 1625 (m), 1543 (w), 1450 (m), 1156 (s), 1100 (s). HRMS (ESI) (m/z): calc'd for C₄₈H₇₈N₅O₉ [M+H]⁺: 868.5794, found: 868.5778.

### Example 92: Cleavage Reaction Progress Monitoring by ¹H NMR Spectroscopy

A solution of enamine N-oxide 32″, 38″, or 39″ (Kang, et al., J. Am. Chem. Soc. 143:5616-5621 (2021)) (280 μL, 10 mM in 25% CD₃OD/d-PBS, pH 7.4; 4 mM final concentration) and a solution of tetrahydroxydiboron (70 μL, 100 mM in DMSO-d₆; 10 mM final concentration) were sequentially added to a solution of caffeine (140 μL, 10 mM in 25% CD₃OD/d-PBS; 2 mM final concentration) in CD₃OD/d-PBS (25% v/v, 210 μL) at room temperature to bring the total volume to 700 μL. The progress of the reaction was monitored by ¹H NMR spectroscopy, and the amount of each species was quantified against the caffeine internal standard. Complete conversion of enamine N-oxide 38″ was observed within 4 min (FIG. 33B). Complete release of a proposed carbamic acid intermediate S14″ occurred within 5 min of diboron treatment (FIG. 34B), and complete formation of p-nitroaniline (24″) was observed within 30 min (FIG. 34C).

### Example 93: Hydroamination and Cleavage Reaction Progress Monitoring by In-Gel Fluorescence

Synthesis of Lys-COT 11″: Lysozyme containing cyclooctyne (Lys-COT 11″) was prepared as previously reported (Kang, et al., J. Am. Chem. Soc. 143:5616-5621 (2021)). Lysozyme (CAS 12650-88-3, 50 mg/mL in deionized H₂O) was diluted into phosphate-buffered saline (PBS, pH 7.4) to a final concentration of 10 mg/mL. A solution of cyclooctyne NHS-ester 42″ (65 μL, 8.5 mM in DMSO) and DMSO (10 μL) were added to the lysozyme solution (250 μL, 10 mg/mL). The reaction solution was incubated for 1 h at room temperature. Excess cyclooctyne NHS-ester 42″ was removed by spin filtration (3 kDa MWCO, 5×1:5 dilution). The concentration of lysozyme was determined by A₂₈₀ measurement in denaturing buffer (pH 7.0, 6 M guanidinium, 30 mM MOPS) on a UV-vis spectrophotometer. The solution was diluted with PBS (pH 7.4) to a final concentration of 0.15 mg/mL or 0.60 mg/mL for labeling experiments. The protein solutions were snap frozen under liquid nitrogen and stored at −20° C.

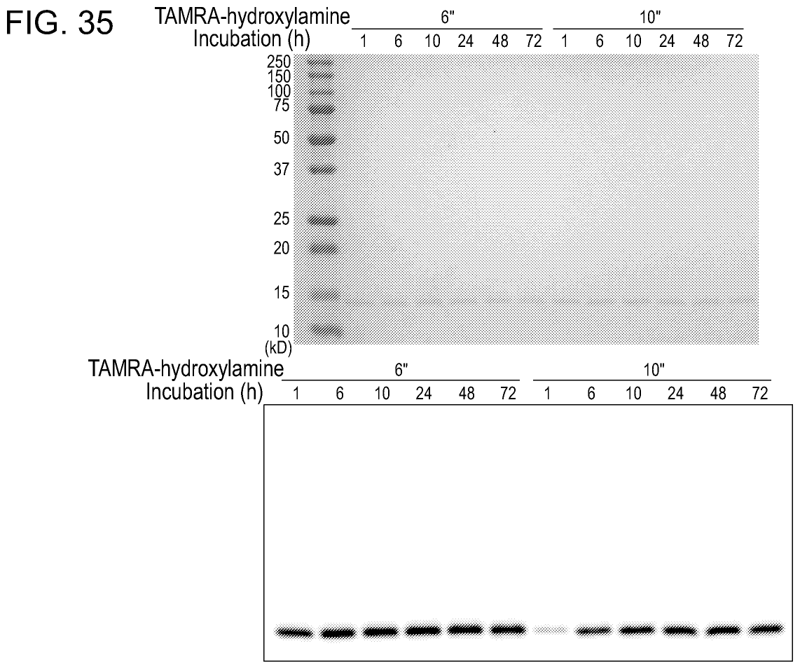

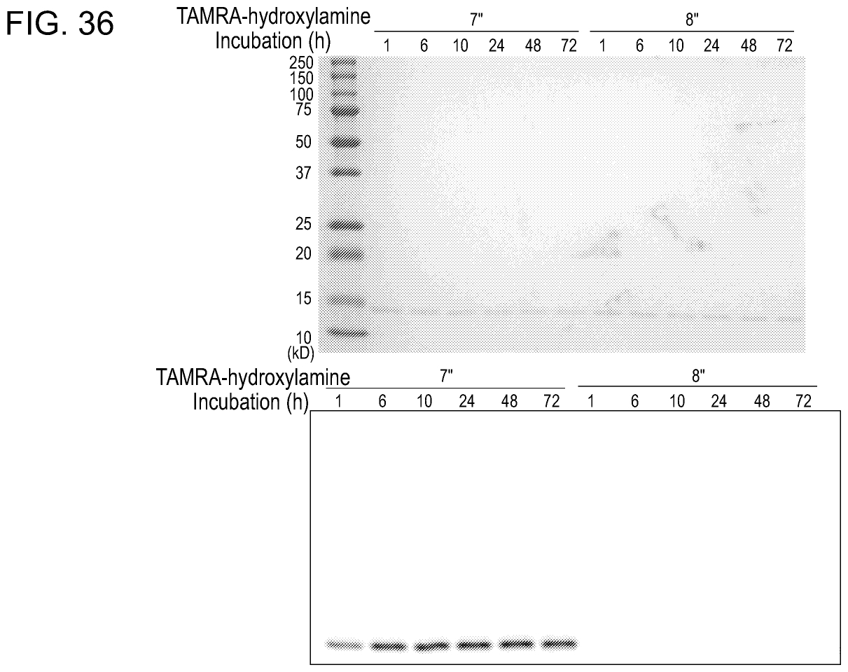

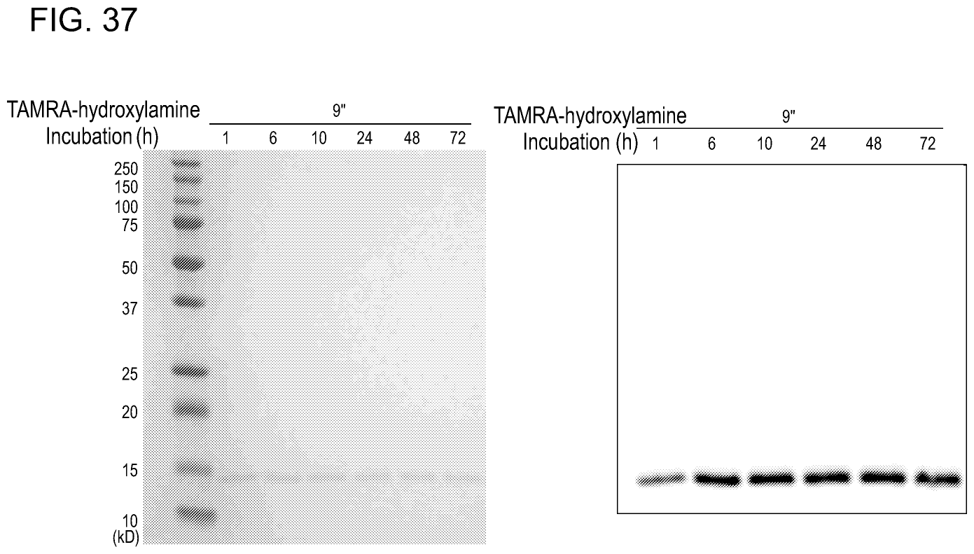

Time-dependent protein labeling experiments: A solution of TAMRA-hydroxylamine 6″ (Kang, et al., J. Am. Chem. Soc. 143:5616-5621 (2021))-8″ and 10″ (1.26 μL, 5 mM in H₂O; 200 μM final concentration) or 9″ (1.26 μL, 5 mM in 50% DMSO/H₂O; 200 μM final concentration) was added to a solution of lysozyme-COT 11″ (30.0 μL, 0.15 mg/mL in PBS, pH 7.4). The reaction mixtures were incubated at room temperature in the dark. At each time point, an aliquot of the reaction mixture (3 uL) was removed, quenched by adding 1×SDS sample buffer (19.5 μL) and N,N-diethylhydroxylamine (7.5 uL, 100 mM in H₂O; 25 mM final concentration), snap frozen in liquid nitrogen, and stored at −80° C. until all samples were ready to be loaded on the gel. After 72 h, all reactions had been quenched. All samples were thawed, and each solution (6 μL) was loaded onto a 15-well 12% SDS-PAGE gel. The gel was run at room temperature and at 175 V for 50 min. In-gel fluorescence was imaged with a Typhoon™ FLA 9500 (GE) at 532 nm with a photomultiplier tube (PMT) setting of 500 V (FIG. 35-FIG. 37).

### Example 94: Stability studies of enamine N-oxide conjugates

A solution of TAMRA-hydroxylamine 6″ and 10″ (2.52 μL, 5 mM in H₂O; 200 μM final concentration) or 9″ (2.52 μL, 5 mM in 50% DMSO/H₂O; 200 μM final concentration) was added to a solution of lysozyme-COT 11″ (60.0 μL, 0.15 mg/mL in PBS, pH 7.4). The reaction mixtures were incubated at room temperature in the dark for 6 h (6″) or 24 h (9″, 10″). An aliquot (2.61 μL) of each reaction mixture was diluted with PBS (pH 7.4), RPMI, or RPMI supplemented with 10% fetal bovine serum (47.4 μL). An aliquot was made for each time point and reaction condition, then all of the reaction mixtures were incubated at room temperature in the dark. At each time point, an aliquot of each solution was snap frozen in liquid nitrogen and stored at −80° C. until all samples were ready to be loaded on the gel. After 24 h, all samples were thawed, and an aliquot of each sample (9.21 μL) was diluted with 5×SDS sample buffer (2.3 μL) and 1×SDS sample buffer (3.5 μL). Each sample (15 μL) was loaded onto a 15-well 12% SDS-PAGE gel. The gel was run at room temperature and at 175 V for 50 min. In-gel fluorescence was imaged with a Typhoon™ FLA 9500 (GE) at 532 nm with a photomultiplier tube (PMT) setting of 500 V (FIG. 38-FIG. 40).

### Example 95: Purification of enamine N-oxide conjugates

Prior to performing the diboron cleavage experiments, enamine N-oxide conjugates 6″_{conj}, 9″_{conj}, and 10″_{conj} were prepared by reaction between lysozyme-COT (100 μL, 0.60 mg/mL) and TAMRA-hydroxylamine (4.17 μL; 5 mM in H₂O stock solution for 6″ and 10″; 5 mM in 50% DMSO/H₂O for 9″). The lysozyme-fluorophore conjugates were purified by gel filtration (PD SpinTrap™ G-25, Cytiva™), and their concentrations were determined based on the A553 absorbance of the TAMRA fluorophore using a UV-vis spectrophotometer.

### Example 96: Screening of diboron derivatives

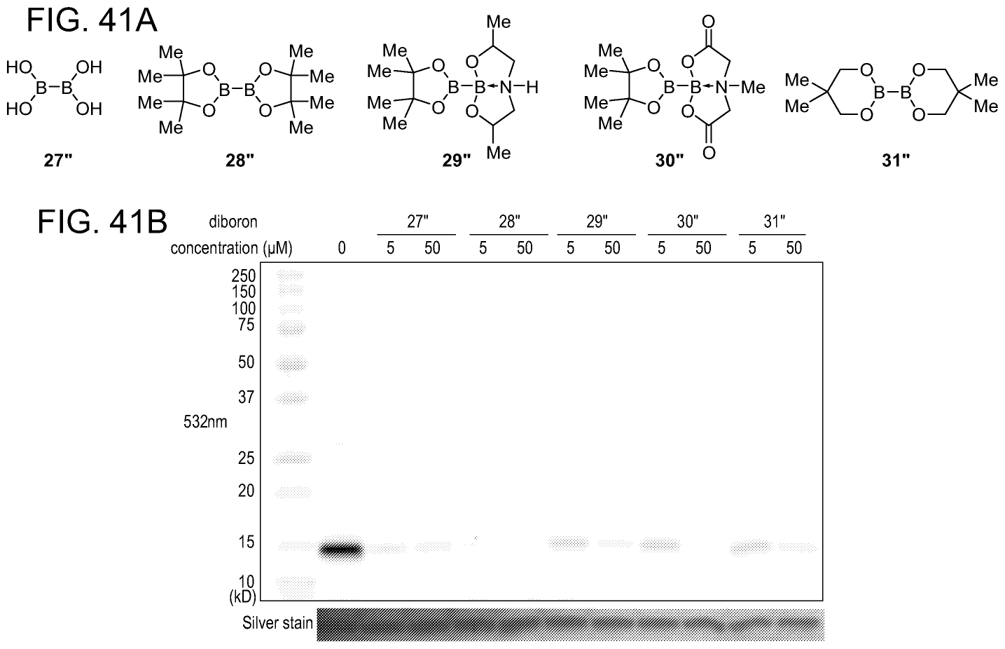

A solution of diborons 27″-31″ (0.34 μL, 125 μM or 1.25 mM in DMSO; 5 or 50 μM final concentration) was added to a solution of lysozyme-fluorophore conjugate 6″_{conj} (8 μL, 0.50 μM in PBS, pH 7.4). The reaction mixtures were incubated at room temperature in the dark. After 60 min, each reaction mixture was quenched by adding trimethylamine N-oxide (0.93 uL, 100 mM in H₂O; 10 mM final concentration) and diluted with 1.6×SDS sample buffer (14.32 μL). Each solution (10 μL) was loaded onto a 15-well 12% SDS-PAGE gel. The gel was run at room temperature and at 175 V for 50 min. In-gel fluorescence was imaged with a Typhoon™ FLA 9500 (GE) at 532 nm with a photomultiplier tube (PMT) setting of 500 V (FIG. 41).

### Example 97: Time and concentration-dependent diboron cleavage on protein

To evaluate the concentration dependence of diboron-mediated cleavage, solutions of bis(pinacolato)diboron (0.34 μL, 125 μM, 250 μM, 500 μM, and 1 mM in DMSO; 5, 10, 20, and 50 μM final concentrations) were independently added to a solution of enamine N-oxide-linked lysozyme-fluorophore conjugate 6″_{conj}, 9″_{conj}, and 10″_{conj} (8 μL, 0.5 μM in PBS, pH 7.4). After 1 h, all samples were quenched with trimethylamine N-oxide (0.93 uL, 100 mM in H₂O; 10 mM final concentration), snap frozen in liquid nitrogen, and stored at −80° C. until all samples were ready to be loaded on the gel.

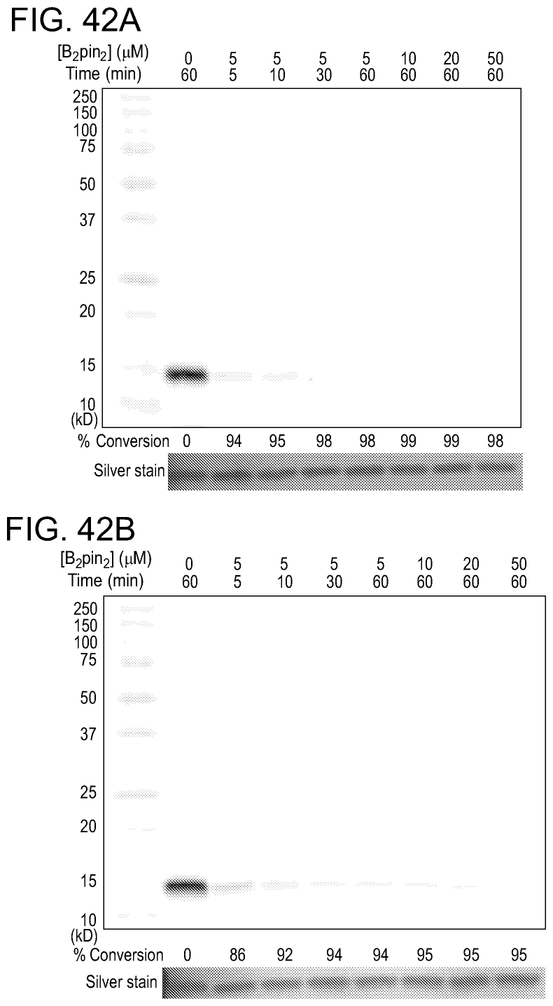

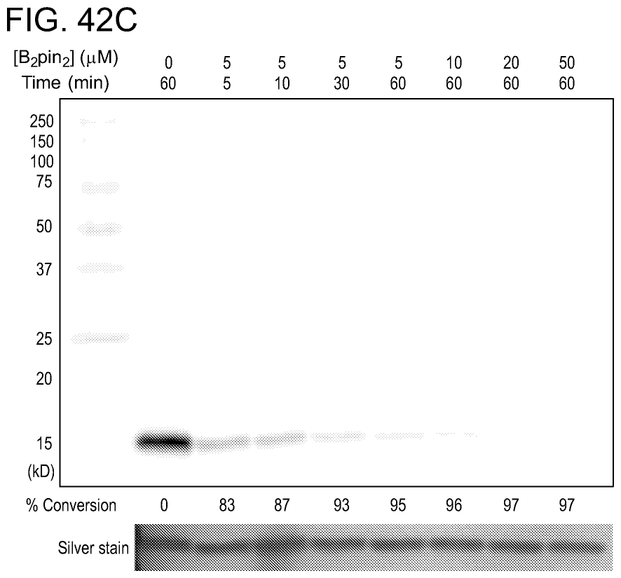

To evaluate the time dependence of diboron-mediated cleavage, bis(pinacolato)diboron (0.34 μL, 125 μM in DMSO; 5 μM final concentration) was added to a solution of enamine N-oxide-linked lysozyme-fluorophore conjugate 6″_{conj}, 9″_{conj}, and 10″_{conj} (8 μL, 0.5 μM in PBS, pH 7.4) in quadruplicate. At each time point, the reaction was quenched with trimethylamine N-oxide (0.93 uL, 100 mM in H₂O; 10 mM final concentration), snap frozen in liquid nitrogen, and stored at −80° C. at each time point (5-60 min) until all samples were ready to be loaded on the gel. All samples were thawed and diluted with 5×SDS sample buffer (2.3 μL) and 1×SDS sample buffer (12.0 μL). Each solution (10 μL) was loaded onto a 15-well 12% SDS-PAGE gel. The gel was run at room temperature and at 175 V for 50 min. In-gel fluorescence was imaged with a Typhoon™ FLA 9500 (GE) at 532 nm with a photomultiplier tube (PMT) setting of 500 V and quantified by ImageJ (FIG. 42).

### Example 98: Reaction monitoring by LCMS

A solution of cyclooctyne 22″ (40 μL, 10 mM in MeOH; 2 mM final concentration) was added to a solution of ⁱPr-NOH 3″ (40 μL, 10 mM in MeOH; 2 mM final concentration) or ⁱBu-NOH 4″ (40 μL, 10 mM in MeOH; 2 mM final concentration) in H₂O (100 μL) containing MeOH (20 μL; 50% v/v MeOH/H₂O final composition). The reaction was monitored by LC-MS analysis (Agilent 1260 Infinity II system, C₁₈ column, 4.6×50 mm, 2.7 μm particle size, 1 mL/min flow rate, eluent: 100% H₂O+0.1% TFA (2 min), gradient 0→100% MeCN/H₂O+0.1% TFA (5 min), 100% MeCN+0.1% TFA (1 min), 100% H₂O+0.1% TFA (1 min)) (FIG. 43).

### Example 99: Intact Mass Spectrometry Analysis

A solution of hydroxylamines 6″ and 10″ (0.42 μL, 5 mM in H₂O) or 9″ (0.42 μL, 5 mM in 50% DMSO/H₂O) was added to a solution of lysozyme-cyclooctyne conjugate 11″ (10 μL, 0.60 mg/mL in PBS, pH 7.4) in duplicate for diboron-mediated cleavage. Deionized H₂O (0.42 μL) was added to lysozyme-cyclooctyne conjugate 11″ (10 μL, 0.60 mg/mL in PBS, pH 7.4) to generate the vehicle control. Unmodified lysozyme (10 μL, 0.60 mg/mL in PBS, pH 7.4) was added to deionized water (0.42 μL) to generate the blank background sample. Reactions were incubated at room temperature for 6 h (6″_{conj}) or 24 h (9″_{conj}, 10″_{conj}) in the dark and diluted with PBS (29.6 μL). Then, a solution of B₂pin₂ (0.40 μL, 2.5 mM in DMSO) or DMSO vehicle control (0.40 μL) was added to the solutions of 6″_{conj}, 9″_{conj}, and 10″_{conj}. DMSO (0.40 μL) was added to the vehicle and blank background samples. The reaction mixtures were incubated at room temperature for 30 min in the dark, snap frozen using liquid nitrogen, and stored at −80° C. until further analysis. ESI-MS analysis was performed on an LTQ XL™ ion trap mass spectrometer (Thermo Scientific™, San Jose, CA) (FIG. 44A-FIG. 44H).

### Example 100: Kinetic Studies

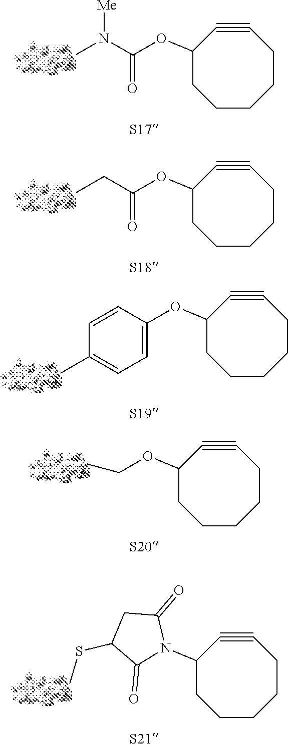 [SMILES: CN(C)C(=O)OC1C#CCCCCC1 ; CCC(=O)OC1C#CCCCCC1 ; Cc1ccc(OC2C#CCCCCC2)cc1 ; CCOC1C#CCCCCC1 ; CSC1CC(=O)N(C2C#CCCCCC2)C1=O]

Synthesis of COT-Lys S17″-S20″ via lysine conjugation: Lysozyme (CAS 12650-88-3, 50 mg/mL in deionized H₂O) was diluted into phosphate-buffered saline (PBS, pH 7.4) to a final concentration of 5 mg/mL. A solution of cyclooctynes 43″-46″ (42.0 μL, 10 mM in DMSO) was added to the lysozyme solution (120 μL, 5 mg/mL). The reaction solution was incubated for 1 h at room temperature. Excess cyclooctynes were removed by gel filtration (PD MidiTrap™ G-25). The concentration of lysozyme was determined by A280 on a NanoDrop™ 8000 spectrophotometer (Thermo Scientific™). The solution was diluted with PBS (pH 7.4) to a final concentration of 0.15 mg/mL or 0.60 mg/mL for further experiments. The protein solutions were snap frozen under liquid nitrogen and stored at −20° C.

Synthesis of COT-Lys S21″ via cysteine conjugation: Lysozyme (CAS 12650-88-3, 50 mg/mL in deionized H₂O) was diluted into phosphate-buffered saline (PBS, pH 7.4) to a final concentration of 5 mg/mL. Lysozyme (100 μL, 5 mg/mL) was reduced in the presence of TCEP (17.5 μL, 3.0 mM; 20 mM in PBS) at room temperature for 2 h, treated with maleimide-cyclooctyne 47″ (35 μL; 10 mM in DMSO), and purified by gel filtration (PD MidiTrap™ G-25) using buffer A. The concentration of lysozyme was determined by A280 on a NanoDrop™ 8000 spectrophotometer (Thermo Scientific™). The solution was diluted with PBS (pH 7.4) to a final concentration of 0.15 mg/mL or 0.60 mg/mL for further experiments. The protein solutions were snap frozen under liquid nitrogen and stored at −20° C.

Synthesis of enamine N-oxide-linked lysozyme-fluorescein conjugate: A solution of fluorescein hydroxylamine 40″ (2.56 μL, 10 mM in deionized water; final concentration 250 μM) was added to a solution of lysozyme-cyclooctyne conjugates 11″ and S17″-S21″ (100 μL, 0.60 mg/mL in PBS, pH 7.4). The reaction mixture was incubated at room temperature in the dark. After 6 h, the product was purified by gel filtration (PD SpinTrap™ G-25) following the manufacturer's protocol to provide enamine N-oxide-linked lysozyme-fluorescein conjugates 41″ and 48″-52″. The concentration of the conjugate was determined based on the A493 absorbance of fluorescein using a UV-vis spectrophotometer.

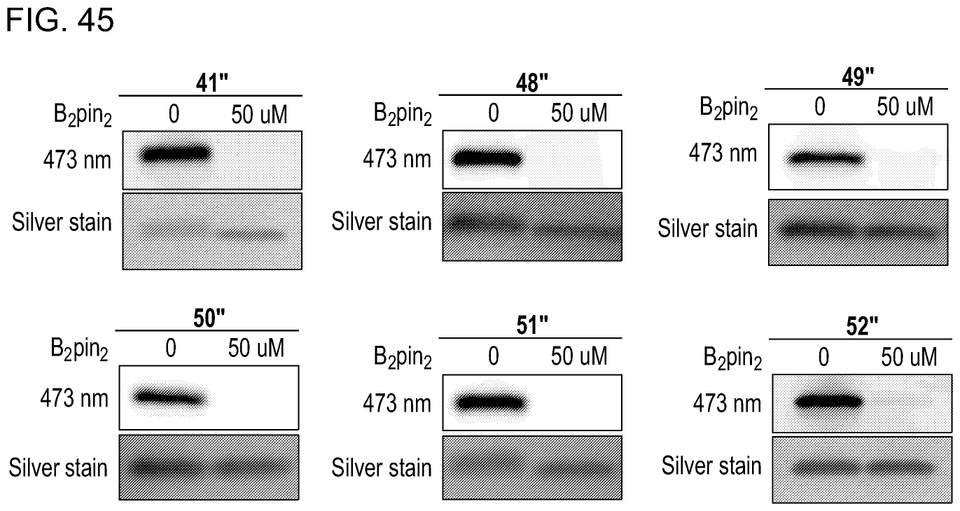

Diboron-mediated cleavage reaction: Enamine N-oxide-linked conjugates 41″ and 48″-52″ (8 μL, 0.50 μM in PBS, pH 7.4) was treated with B₂pin₂ (0.34 μL, 1.25 mM in DMSO; 50 μM final concentration) or DMSO (0.34 μL) as a vehicle. The reaction mixtures were incubated at room temperature in the dark. After 60 min, the reactions were quenched with trimethylamine N-oxide (0.93 μL, 100 mM in deionized water; 10 mM final concentration), 5×SDS sample buffer (2.31 μL) was added, and the samples were diluted with 1×SDS sample buffer (12.0 μL). Each solution (10 μL) was loaded onto a 15-well 12% SDS-PAGE gel. The gel was run at room temperature and at 175 V for 45 min. In-gel fluorescence was imaged with a Typhoon™ FLA 9500 (GE) at 473 nm with a photomultiplier tube (PMT) setting of 500 V (FIG. 45).

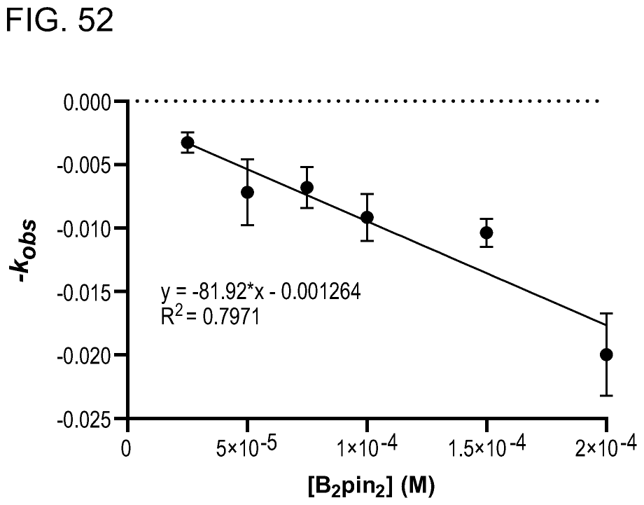

Kinetics assay (pseudo-first order kinetics model): The kinetics assay was performed using a microplate reader (Clariostar Plus, BMG Labtech) with a filter setting of Ex 482-16/LP 504/Em 530-40. First, the parameters for the fluorescence polarization experiments had to be determined. Enamine N-oxide-linked lysozyme-fluorescein conjugate 41″ (20 μL, 0.50 μM in PBS, pH 7.4) were added to separate wells of a 384-well plate. Either B₂pin₂ (2.02 μL, 0.25-2.0 mM in DMSO, Table 2) or DMSO vehicle control (2.02 μL) was added into each well containing lysozyme-fluorescein conjugates 41″. The plate was incubated at room temperature until there was no change in fluorescence polarization to determine the end point of each reaction. The mP was set to 100 based on the end point experiment, and the gain was adjusted prior to the kinetics measurement. For the kinetics measurements, B₂pin₂ (2.02 μL, 0.25-2.0 mM in DMSO, Table 2) was added into each well containing lysozyme-fluorescein conjugate, the gains were adjusted, and the plate was shaken for 10 sec at 300 rpm prior to measurement. Fluorescence polarization was measured every 15 sec. Each assay was performed in triplicate (FIG. 52).

TABLE 2
Kinetic Assay

| Stock concentration | | Final | |
|---|---|---|---|
| of B₂pin₂ | | concentration | |
| 0.25 | mM | 25 | μM |
| 0.50 | mM | 50 | μM |
| 0.75 | mM | 75 | μM |
| 1.0 | mM | 100 | μM |
| 1.5 | mM | 150 | μM |
| 2.0 | mM | 200 | μM |

Kinetics under different pHs: The kinetics assay was performed using a microplate reader (Clariostar Plus, BMG Labtech) with a filter setting of Ex 482-16/LP 504/Em 530-40. First, the parameters for the fluorescence polarization experiments had to be determined. Enamine N-oxide-linked lysozyme-fluorescein conjugate 41″ (5.94 μL, 10.6 μM in PBS, pH 7.4) was diluted with PBS (120 μL, pH=4, 6, 8, or 10) to adjust the pH. Each solution (20.0 μL) was added to separate wells of a 384-well plate. Either B₂pin₂ (2.02 μL, 8.0 mM in DMSO; 800 μM final concentration) or DMSO vehicle control (2.02 μL) was added into each well containing lysozyme-fluorescein conjugate 41″. The plate was incubated at room temperature until there was no further change in fluorescence polarization to determine the end point of each reaction. The mP was set to 100 based on the end point experiment, and the gain was adjusted prior to the kinetics measurement. To conduct the kinetics measurements, B₂pin₂ (2.02 μL, 1.0 mM in DMSO; 100 μM final concentration) was added to each well containing conjugate 41″, gains were adjusted, and the plate was shaken for 10 sec at 300 rpm prior to measurement. Fluorescence polarization was measured every 15 sec. Each assay was performed in triplicate.

Kinetics in different buffer systems: The kinetics assay was performed using a microplate reader (Clariostar Plus, BMG Labtech) with a filter setting of Ex 482-16/LP 504/Em 530-40. First, the parameters for the fluorescence polarization experiments had to be determined. Enamine N-oxide-linked lysozyme-fluorescein conjugate 41″ (10 μL, 11.6 μM in PBS, pH 7.4) was diluted with citrate buffer (222 μL, pH 6.0, 10 mM citric acid), tris buffer (222 μL, pH 7.4, 50 mM tris), HEPES buffer (222 μL, pH 7.4, 50 mM HEPES), or RPMI (222 μL) at a final concentration of 500 nM. Each solution (20.0 μL) was added to separate wells of a 384-well plate. Either B₂pin₂ (2.02 μL, 0.5 mM in DMSO; 50 μM final concentration) or DMSO vehicle control (2.02 μL) was added into each well containing lysozyme-fluorescein conjugate 41″. The plate was incubated at room temperature until there was no further change in fluorescence polarization to determine the end point of each reaction. The mP was set to 100 based on the end point experiment, and the gain was adjusted prior to the kinetics measurement. For the kinetics measurements, B₂pin₂ (2.02 μL, 0.5 mM in DMSO; 50 μM final concentration) was added to each well containing conjugate 41″, gains were adjusted, and the plate was shaken for 10 sec at 300 rpm prior to measurement. Fluorescence polarization was measured every 15 sec. Each assay was performed in triplicate.

Kinetics studies with different diborons: The kinetics assay was performed using a microplate reader (Clariostar Plus, BMG Labtech) with a filter setting of Ex 482-16/LP 504/Em 530-40. First, the parameters for the fluorescence polarization experiments had to be determined. Enamine N-oxide-linked lysozyme-fluorescein conjugate 41″ (20 μL, 0.50 μM in PBS, pH 7.4) were added to separate wells of a 384-well plate. Either diboron 27″-31″ (2.02 μL, 0.50 mM in DMSO, Table 3) or DMSO vehicle control (2.02 μL) was added into each well containing lysozyme-fluorescein conjugates 41″. The plate was incubated at room temperature until there was no change in fluorescence polarization to determine the end point of each reaction. The mP was set to 100 based on the end point experiment, and the gain was adjusted prior to the kinetics measurement. For the kinetics measurements, diborons 27″-31″ (2.02 μL, 0.50 mM in DMSO; 50 μM final concentration) was added to each well containing lysozyme-fluorescein conjugate, the gains were adjusted, and the plate was shaken for 10 sec at 300 rpm prior to measurement. Fluorescence polarization was measured every 15 sec. Each assay was performed in triplicate. (FIG. 46)

Kinetics studies of enamine N-oxide derivatives: The kinetics assay was performed using a microplate reader (Clariostar Plus, BMG Labtech) with a filter setting of Ex 482-16/LP 504/Em 530-40. First, the parameters for the fluorescence polarization experiments had to be determined. Enamine N-oxide-linked lysozyme-fluorescein conjugates 41″ and 48″-52″ were diluted with PBS (pH 7.4) to a final concentration of 500 nM. Each solution (20.0 μL) was added to separate wells of a 384-well plate. Either B₂pin₂ (2.02 μL, 0.5 mM in DMSO; 50 μM final concentration) or DMSO vehicle control (2.02 μL) was added into each well containing lysozyme-fluorescein conjugate. The plate was incubated at room temperature until there was no further change in fluorescence polarization to determine the end point of each reaction. The mP was set to 100 based on the end point experiment, and the gain was adjusted prior to the kinetics measurement. For the kinetics measurements, B₂pin₂ (2.02 μL, 0.5 mM in DMSO; 50 μM final concentration) was added to each well containing conjugate, gains were adjusted, and the plate was shaken for 10 sec at 300 rpm prior to measurement. Fluorescence polarization was measured every 15 sec. Each assay was performed in triplicate.

### Example 101: Stability of Enamine N-Oxide Antibody Conjugates

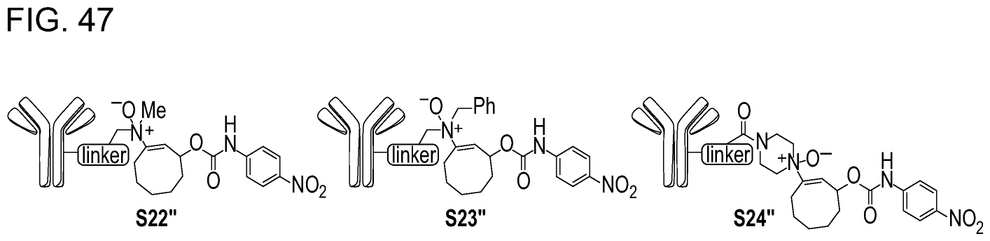

Synthesis of antibody-nitroaniline conjugates S22″-S24″: Maleimide-hydroxylamines S10″, S11″, or 56″ (50 μL, 10 mM) were added to a solution of cyclooctynyl p-nitrophenyl carbonate (57″, 150 μL, 10 mM in DMSO) in deionized water (50 μL). The reaction mixtures were incubated at room temperature for 12 h to form enamine N-oxide products. Human IgG isotype control (Invitrogen 02-7102, 5 mg/mL in PBS, pH 7.4) was diluted into buffer A (100 mM phosphate, 5 mM EDTA, pH 7.4) at a final concentration of 3.3 mg/mL. The antibody (1.60 mL, 3.3 mg/mL) was reduced in the presence of TCEP (41 μL, 20 mM in buffer A; 500 μM final concentration) at 37° C. for 1 h. Each enamine N-oxide-containing solution (173 μL; 500 μM final concentration of enamine N-oxides) was then added to the solution of reduced antibody (520 μL). The reaction mixtures were incubated at room temperature for 2 h and purified by gel filtration (PD MidiTrap™ G-25) and spin filtration (Amicon Ultra 4, UFC801024, 10 kDa MWCO) using PBS (pH 7.4) to provide antibody-nitroaniline conjugates S22″-S24″ (FIG. 47). The concentration and loading of each conjugate were determined by A324 on a NanoDrop™ 8000 spectrophotometer (Thermo Scientific™)

Antibody conjugate stability assay: Each antibody-p-nitroaniline conjugate (S22″ 18.80 μM, S23″ 16.50 μM, and S24″ 22.28 μM) was diluted into RPMI supplemented with 5% of heat-inactivated human serum (Sigma) to a final volume of 300 μL and a final concentration of conjugate at 3.30 μM. Each solution of antibody-p-nitroaniline conjugate (50 μL, 3.30 μM) was added into separate wells of a 96-well plate. The plate was incubated at 37° C. under ambient atmosphere with 5% CO₂. At each time point, a solution of 4-nitronaphthylamine (50 μL, 10 μM in acetonitrile; internal standard for HPLC analysis) was added to the well and transferred to a microcentrifuge tube. Samples were centrifuged at 20,000×g for 10 min at 4° C. The supernatant was transferred to a vial for HPLC analysis (Agilent 1260 Infinity system, C₁₈ column, 4.6×250 mm, 5 μm particle size, 1 mL/min flow rate, eluent: 100% H₂O+0.11% TFA (1 min), gradient 0→100% MeCN/H₂O+0.1% TFA (4 min), 100% MeCN+0.1% TFA (1 min), 100% H₂O+0.1% TFA (1 min)), and the amount of p-nitroaniline was quantified based on the relative area under the curve in the UV chromatogram at 381 nm compared to 4-nitronaphthylamine.

TABLE 3
Stability study of enamine N-oxide antibody conjugates.

| | % released | | |
|---|---|---|---|
| | Incubation time | | |
| Substrate | 18 h | 40 h | 70 h |
| S22″ | 1.7 | 3.0 | 4.1 |
| S23″ | 10 | 16 | 20 |
| S24″ | 0.53 | 1.4 | 1.6 |

Antibody conjugate stability assay in the presence of cells: SK-BR-3 cell were seeded at a density of 10,000 cells per well in media [100 μL, RPMI supplemented with 5% heat-inactivated human serum (Sigma), penicillin (100 units/mL), streptomycin (0.1 mg/mL)] in a 96-well plate. PBS (100 μL) was added to the edge wells. The cells were incubated at 37° C. under ambient atmosphere with 5% CO₂. After 24 h, the media was aspirated and replaced with media [50 μL, RPMI supplemented with 5% heat-inactivated human serum (Sigma), penicillin (100 units/mL), streptomycin (0.1 mg/mL)] containing antibody-p-nitroaniline conjugate S24″ (3.90 μM). The plate was incubated at 37° C. under ambient atmosphere with 5% CO₂. At each time point, a solution of 4-nitronaphthylamine (50 μL, 10 μM in acetonitrile, internal standard for HPLC analysis) was added to the well and transferred to a microcentrifuge tube. Samples were centrifuged at 20,000×g for 10 min at 4° C. The supernatant was transferred to a vial for HPLC analysis (Agilent 1260 Infinity system, C₁₈ column, 4.6×250 mm, 5 μm particle size, 1 mL/min flow rate, eluent: 100% H₂O+0.1% TFA (1 min), gradient 0→100% MeCN/H₂O+0.1% TFA (4 min), 100% MeCN+0.1% TFA (1 min), 100% H₂O+0.1% TFA (1 min)), and the amount of p-nitroaniline was quantified based on the relative area under the curve in the UV chromatogram at 381 nm compared to 4-nitronaphthylamine.

TABLE 4
Stability study of enamine N-oxide antibody conjugate S24″.

| | % released | | |
|---|---|---|---|
| | Incubation time | | |
| Conjugate | 2 h | 18 h | 42 h |
| S24″ | 0.14 | 1.0 | 2.2 |

### Example 102: Synthesis of Antibody-Drug Conjugates

General procedure for the synthesis of antibody-drug conjugates: Antibody (Human IgG isotype control: Invitrogen 02-7102, 5 mg/mL in PBS, pH 7.4; Trastuzumab: Biosynth FT65040, 20 mg/mL in PBS) was diluted into buffer A (100 mM phosphate, 5 mM EDTA, pH 7.4) at a final concentration of 3.3 mg/mL. The antibody (3.3 mg/mL) was reduced with TCEP (20 mM in buffer A stock solution; 500 μM final concentration) at 37° C. for 1 h, treated with maleimide-hydroxylamine 56″ (10.0 equiv), and purified by gel filtration (PD MidiTrap™ G-25) using buffer A. The resulting hydroxylamine-modified antibodies were then treated with cyclooctyne-MMAE 58″ (10 mM in DMSO stock solution; 300 μM final concentration). The reaction mixtures were incubated at room temperature for 12 h and purified by gel filtration (PD MidiTrap™ G-25) and spin filtration (Amicon Ultra 4, UFC801024, 10 kDa MWCO) using PBS (pH 7.4). The concentration of antibody-drug conjugate was determined at A280 on a NanoDrop™ 8000 spectrophotometer (Thermo Scientific™)

Determination of drug antibody ratio (DAR): Purified antibody-drug conjugate (25 μL) was treated with B₂pin₂ (2.78 μL, 10 mM in DMSO; 1 mM final concentration) or DMSO vehicle (2.78 μL). The reaction mixtures were incubated at room temperature. After 30 min, acetonitrile (27.8 μL) was added to precipitate the antibodies. The resulting solutions were centrifuged at 20,000×g for 10 min at 4° C. The supernatant was transferred to a vial and analyzed by HPLC (Agilent 1260 Infinity system, C₁₈ column, 4.6×250 mm, 5 μm particle size, 1 mL/min flow rate, eluent: 100% H₂O+0.11% TFA (1 min), gradient 0→100% MeCN/H₂O+0.1% TFA (4 min), 100% MeCN+0.1% TFA (1 min), 100% H₂O+0.1% TFA (1 min)). The amount of MMAE was quantified based on the calibration curve obtained at 220 nm and used to determine the DAR (1.55 for human IgG isotype control-based ADC 62″ and 2.42 for trastuzumab-MMAE 61″).

### Example 103: Cell Viability Assay

Cell culture: Cells were cultured in RPMI (SK-BR-3) or DMEM (MDA-MB-231) containing 10% FBS (Sigma), 100 units/mL penicillin, and 0.1 mg/mL streptomycin (Sigma) in a humidified chamber at 37° C. under an ambient atmosphere with 5% CO₂. Cells were passaged and dissociated with 0.25% trypsin, 0.1% EDTA in HBSS (Corning). All cells tested negative for mycobacteria with the MycoAlert™ PLUS Mycoplasma Detection Kit (Lonza) following the manufacturer's protocol.

Cell viability assay: SK-BR-3 or MDA-MB-231 cell were seeded at a density of 5,000-10,000 cells per well in media [100 μL, RPMI (SK-BR-3) or DMEM (MDA-MB-231) supplemented with 5% heat-inactivated human serum (Sigma), penicillin (100 units/mL), streptomycin (0.1 mg/mL)] in an opaque 96-well plate. PBS (100 μL) was added to the edge wells. The cells were incubated at 37° C. under ambient atmosphere with 5% CO₂. After 24 h, the media was aspirated and replaced with media [100 μL, RPMI (SK-BR-3) or DMEM (MDA-MB-231) supplemented with 5% heat-inactivated human serum (Sigma), penicillin (100 units/mL), streptomycin (0.1 mg/mL)] containing various treatments [Human IgG isotype control, trastuzumab, ADCs 61″ and 62″, and MMAE starting at 100 nM with 4-fold serial dilution across nine wells; ADCs 61″ and 62″ starting at 100 nM with 4-fold serial dilution across 9 wells each combined with 50 μM B₂pin₂; or diboron reagents starting at 500 μM (B₂pin₂) or 1 mM (B₂(OH)₄) with 4-fold serial dilution across nine wells]. Vehicle controls corresponding to the treatment in parentheses consisted of 0.7% v/v PBS (Human IgG isotype control, trastuzumab, ADC), 0.7% v/v PBS with 0.5% v/v DMSO (ADC+B₂pin₂), or 0.5% v/v DMSO (diboron reagents). The plates were incubated at 37° C. with 5% CO₂ for 72 h (SK-BR-3) or 96 h (MDA-MB-231). After the plates were equilibrated at room temperature, CellTiter-Glo™ 2.0 reagent (50 μL, Promega™) was added to each well and mixed gently. The plates were incubated at room temperature for 10 min to stabilize the luminescence signal and analyzed by a microplate reader (Clariostar Plus, BMG Labtech) (FIG. 48 and FIG. 49).

### Example 104: Traceless protein modification

Synthesis of Lys-COT 64″: Lysozyme (CAS 12650-88-3, 50 mg/mL in deionized H₂O) was diluted into phosphate-buffered saline (PBS, pH 7.4) to a final concentration of 5 mg/mL. A solution of cyclooctyne 57″ (42.0 μL, 10 mM in DMSO) was added to the lysozyme solution (120 μL, 5 mg/mL). The reaction solution was incubated for 1 h at room temperature. Excess cyclooctyne was removed by gel filtration (PD MidiTrap™ G-25). The concentration of lysozyme was determined by A280 on a NanoDrop™ 8000 spectrophotometer (Thermo Scientific™). The solution was diluted with PBS (pH 7.4) to a final concentration of 0.15 mg/mL or 0.60 mg/mL for further experiments. The protein solutions were snap frozen under liquid nitrogen and stored at −20° C.

Synthesis of enamine N-oxide-linked lysozyme-fluorescein conjugate 65″: A solution of fluorescein hydroxylamine 40″ (2.56 μL, 10 mM in deionized water; final concentration 250 μM) was added to a solution of lysozyme-cyclooctyne conjugate 64″ (100 μL, 0.60 mg/mL in PBS, pH 7.4). The reaction mixture was incubated at room temperature in the dark. After 6 h, the product was purified by gel filtration (PD SpinTrap™ G-25) following the manufacturer's protocol to provide enamine N-oxide-linked lysozyme-fluorescein conjugate 65″. The concentration of the conjugate was determined based on the A493 absorbance of fluorescein using a UV-vis spectrophotometer.

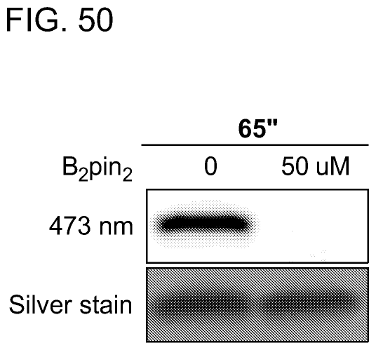

Diboron cleavage and in-gel fluorescence analysis: Enamine N-oxide-linked conjugate 65″ (8 μL, 0.50 μM in PBS, pH 7.4) were treated with B₂pin₂ (0.34 μL, 1.25 mM in DMSO; 50 μM final concentration) or DMSO (0.34 μL) as a vehicle. The reaction mixtures were incubated at room temperature in the dark. After 60 min, the reactions were quenched with trimethylamine N-oxide (0.93 μL, 100 mM in deionized water; 10 mM final concentration), 5×SDS sample buffer (2.31 μL) was added, and the samples were diluted with 1×SDS sample buffer (12.0 μL). Each solution (10 μL) was loaded onto a 15-well 12% SDS-PAGE gel. The gel was run at room temperature and at 175 V for 45 min. In-gel fluorescence was imaged with a Typhoon™ FLA 9500 (GE) at 473 nm with a photomultiplier tube (PMT) setting of 500 V(FIG. 50).

### Example 105: Intact Mass Spectrometry Analysis

A solution of fluorescein hydroxylamine 40″ (0.42 μL, 5 mM in H₂O) was added to a solution of lysozyme-cyclooctyne conjugate 64″ (10 μL, 0.60 mg/mL in PBS, pH 7.4) in duplicate for diboron-mediated cleavage. Deionized H₂O (0.42 μL) was added to lysozyme-cyclooctyne conjugate 64″ (10 μL, 0.60 mg/mL in PBS, pH 7.4) to generate the vehicle control. Unmodified lysozyme (10 μL, 0.60 mg/mL in PBS, pH 7.4) was added to deionized water (0.42 μL) to generate the blank background sample. Reaction was incubated at room temperature for 6 h in the dark and diluted with PBS (29.6 μL). Then, a solution of B₂pin₂ (0.40 μL, 2.5 mM in DMSO) or DMSO vehicle control (0.40 μL) was added to the solutions of enamine N-oxide-linked conjugate 65″. DMSO (0.40 μL) was added to the vehicle and blank background samples. The reaction mixtures were incubated at room temperature for 30 min in the dark, snap frozen using liquid nitrogen, and stored at −80° C. until further analysis. ESI-MS analysis was performed on an LTQ XL™ ion trap mass spectrometer (Thermo Scientific™, San Jose, CA).

### Example 106: Computational Details

All calculations were conducted with Gaussian 09 software (Frisch, et al., Gaussian 16 Rev. C.01, Wallingford, CT (2019)). Geometry optimization of all species was performed using the M06-2X functional (Zhao, et al., Theor. Chem. Acc. 120:215-241 (2008)) with the 6-31G(d,p) basis set. Frequency analysis was carried out to ensure the stationary point was either a minimum or a transition state. Intrinsic reaction coordinates were computed for all transition states. Single-point calculations were carried out using the M06-2X functional with the 6-311G(2d,p) basis set. The 3D image in FIG. 3C was generated by using CYLview (Cylview, 1.0b, Legault, C. Y. Universite de Sherbrooke (2009)).
Cartesian coordinates of optimized structures (4)

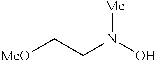

| | | | |
|---|---|---|---|
| H | 1.71683400 | 1.92976600 | 0.41213900 |
| N | 1.52542200 | 0.04201000 | 0.37652400 |
| O | 1.67161300 | 1.29004300 | −0.30754300 |
| C | 0.32620800 | −0.57557300 | −0.17485500 |
| H | 0.37254600 | −0.63332600 | −1.27363600 |
| H | 0.26444300 | −1.59377700 | 0.22162700 |
| C | −0.91486000 | 0.18945900 | 0.24319900 |
| H | −0.95116600 | 0.26279800 | 1.34255200 |
| H | −0.87822400 | 1.21108000 | −0.16397500 |
| O | −2.03103400 | −0.50706000 | −0.25218600 |
| C | −3.23644600 | 0.14139600 | 0.05395400 |
| H | −3.27278600 | 1.15067600 | −0.38202500 |
| H | −4.04862500 | −0.45554300 | −0.36397300 |
| H | −3.38136400 | 0.23119500 | 1.14085800 |
| C | 2.71215400 | −0.73299100 | 0.04353400 |
| H | 3.59229800 | −0.21358300 | 0.42571000 |
| H | 2.63845900 | −1.70874500 | 0.52966300 |
| H | 2.82267000 | −0.87221900 | −1.04177400 |

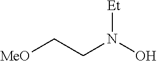

| | | | |
|---|---|---|---|
| H | 1.11320000 | 2.27278200 | −0.12082100 |
| N | 1.05557400 | 0.48558200 | 0.51053900 |
| O | 1.19750600 | 1.42042100 | −0.56297000 |
| C | −0.01383700 | −0.42194300 | 0.11344200 |
| H | 0.11525200 | −0.79018900 | −0.91384200 |
| H | 0.00498100 | −1.28015800 | 0.79336500 |
| C | 2.34976500 | −0.18407800 | 0.66420100 |
| H | 3.06648500 | 0.59391400 | 0.94370900 |
| C | 2.85185200 | −0.94030500 | −0.56344900 |
| H | 2.23295900 | −1.81403300 | −0.78358400 |
| H | 2.84598100 | −0.28167100 | −1.43473800 |
| H | 3.87446500 | −1.28906700 | −0.39916600 |
| C | −1.35675500 | 0.27441600 | 0.22583500 |
| H | −1.51205000 | 0.61901800 | 1.26103100 |
| H | −1.36788400 | 1.15870600 | −0.43025500 |
| O | −2.35080200 | −0.64356600 | −0.15474900 |
| C | −3.63524600 | −0.08262500 | −0.10473200 |
| H | −3.72440800 | 0.78024600 | −0.78123600 |
| H | −4.34463500 | −0.85159000 | −0.41479800 |
| H | −3.89079800 | 0.25038400 | 0.91224000 |
| H | 2.24911800 | −0.85504200 | 1.52429000 |

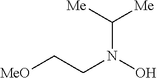

| | | | |
|---|---|---|---|
| H | 0.85725000 | −1.95031800 | 0.87929300 |
| N | 0.72726800 | −0.40623900 | −0.21682900 |
| O | 0.84708100 | −1.00689200 | 1.07638900 |
| C | −0.42662900 | 0.47981200 | −0.13612600 |
| H | −0.40779000 | 1.10267500 | 0.76882200 |
| H | −0.40754900 | 1.13892000 | −1.01026500 |
| C | 1.98997800 | 0.30265800 | −0.49321200 |
| H | 1.83064000 | 0.77024000 | −1.47310700 |
| C | 2.33763800 | 1.37903500 | 0.53661800 |
| H | 3.31697800 | 1.81135200 | 0.31463400 |
| H | 1.60735300 | 2.19217800 | 0.54217200 |
| H | 2.37234000 | 0.93572700 | 1.53517400 |
| C | 3.10844300 | −0.72482200 | −0.62033200 |
| H | 3.30439200 | −1.18760100 | 0.35067300 |
| H | 2.83059700 | −1.50545100 | −1.33328800 |
| H | 4.03059700 | −0.24916600 | −0.96328100 |
| C | −1.70838100 | −0.33195000 | −0.15159700 |
| H | −1.75823400 | −0.93253400 | −1.07420300 |
| H | −1.71461500 | −1.02534700 | 0.70388500 |
| O | −2.78721100 | 0.56590200 | −0.07533000 |
| C | −4.02441900 | −0.09448000 | −0.06181900 |
| H | −4.11124900 | −0.76920100 | 0.80269900 |
| H | −4.80480100 | 0.66559000 | 0.00136400 |
| H | −4.17552100 | −0.68699000 | −0.97643200 |

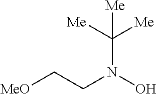

| | | | |
|---|---|---|---|
| N | −0.52290000 | 0.36394600 | −0.34087200 |
| O | −0.50489800 | 1.68315300 | 0.21684300 |
| C | 0.60832400 | −0.31480500 | 0.28200700 |
| H | 0.61523300 | −0.18833700 | 1.37358800 |
| H | 0.54757300 | −1.38163400 | 0.05624600 |
| C | −1.85365000 | −0.22894500 | −0.04051500 |
| C | −2.13251000 | −0.32056200 | 1.46351600 |
| H | −3.16115300 | −0.65020500 | 1.63646000 |
| H | −1.46678800 | −1.03794200 | 1.95240500 |
| H | −1.99705500 | 0.65815000 | 1.93071200 |
| C | −2.89768300 | 0.66801700 | −0.71098000 |
| H | −2.91640200 | 1.65639100 | −0.24805800 |
| H | −2.67055800 | 0.77776400 | −1.77604500 |
| H | −3.89202400 | 0.22429900 | −0.61243900 |
| C | 1.91024700 | 0.21904500 | −0.28790800 |
| H | 1.91275200 | 0.09505600 | −1.38316600 |
| H | 2.00213700 | 1.29345300 | −0.06796400 |
| O | 2.96188900 | −0.50336300 | 0.30319900 |
| C | 4.21825100 | −0.06460400 | −0.13908300 |
| H | 4.38391000 | 0.99508100 | 0.10540000 |
| H | 4.97573000 | −0.66755600 | 0.36432700 |
| H | 4.32864100 | −0.18728000 | −1.22687900 |
| C | −1.90996300 | −1.61418000 | −0.69117500 |
| H | −2.94004600 | −1.98055600 | −0.67554700 |
| H | −1.57653400 | −1.55831700 | −1.73143500 |
| H | −1.29535700 | −2.34806600 | −0.16418300 |
| H | −0.50379400 | 2.24995800 | −0.56283100 |

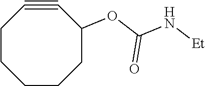 [SMILES: CCNC(=O)OC1C#CCCCCC1]

| | | | |
|---|---|---|---|
| C | 3.25358900 | 1.22836300 | 0.58818700 |
| C | 2.04246500 | 1.59947800 | −0.31776800 |
| C | 2.58391200 | −1.43832100 | −0.02117700 |
| C | 0.64947300 | 1.08846000 | 0.10572200 |
| C | 1.42043900 | −1.17699200 | −0.20801300 |
| C | 0.28274300 | −0.29589400 | −0.46191900 |
| H | 2.89019100 | 0.84018100 | 1.54781400 |
| H | 2.24209300 | 1.27992800 | −1.34839100 |
| H | 0.59020400 | 1.01758800 | 1.19787700 |
| H | 3.78375700 | 2.15629000 | 0.82442300 |
| H | 1.97999600 | 2.69109500 | −0.35735800 |
| H | −0.12760700 | 1.78388700 | −0.22539000 |
| H | 0.08624200 | −0.21383500 | −1.53595700 |
| C | 4.29926200 | 0.24774100 | 0.01240900 |
| H | 5.28660100 | 0.47596400 | 0.42817200 |
| H | 4.37280500 | 0.39604200 | −1.07078100 |
| C | 3.99863600 | −1.24570900 | 0.28682300 |
| H | 4.17924800 | −1.47649600 | 1.34290700 |
| O | −0.89699800 | −0.80373100 | 0.17372500 |
| C | −2.06282900 | −0.33106600 | −0.32770200 |
| O | −2.14674700 | 0.46843700 | −1.23859400 |
| N | −3.11643300 | −0.88903300 | 0.31820700 |
| H | −2.91685100 | −1.44694300 | 1.13359200 |
| H | 4.65648800 | −1.88907100 | −0.30457600 |
| C | −4.46448400 | −0.41690900 | 0.05262600 |
| H | −4.56410200 | −0.31733100 | −1.03078600 |
| H | −5.15881400 | −1.19469400 | 0.38069300 |
| C | −4.77026800 | 0.91578300 | 0.72876400 |
| H | −4.07209400 | 1.67560000 | 0.37008400 |
| H | −5.78716600 | 1.24280900 | 0.49653400 |
| H | −4.67363500 | 0.83497900 | 1.81494000 |

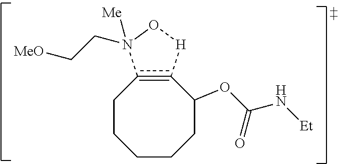

| | | | |
|---|---|---|---|
| C | 0.94134400 | 2.96568500 | 1.10918300 |
| C | −0.59112700 | 2.79093000 | 1.13668900 |
| C | 1.33010600 | 0.76019500 | −0.86632800 |
| C | −1.17836800 | 1.37552200 | 1.08045700 |
| C | 0.16926700 | 0.31638500 | −0.73200400 |
| C | −1.17618400 | 0.71288600 | −0.30025400 |
| H | 1.41205300 | 2.13651300 | 1.65513600 |
| H | −1.04039300 | 3.39863200 | 0.33981000 |
| H | −0.65192300 | 0.71625500 | 1.78144400 |
| H | 1.16809300 | 3.86765900 | 1.68652900 |
| H | −0.94914600 | 3.22937000 | 2.07443100 |
| H | −2.22448200 | 1.43325600 | 1.39717400 |
| H | −1.63338400 | 1.39373200 | −1.02707700 |
| C | 1.60845900 | 3.11841000 | −0.27367300 |
| H | 2.34973000 | 3.92370000 | −0.23889400 |
| H | 0.85889300 | 3.41639600 | −1.01599900 |
| C | 2.31724400 | 1.85178700 | −0.77240000 |
| H | 3.12105400 | 1.58325300 | −0.07238700 |
| O | −2.00545600 | −0.47510000 | −0.24744800 |
| C | −3.33187900 | −0.26202300 | −0.36071700 |
| O | −3.85325800 | 0.83079100 | −0.48024400 |
| N | −4.00888300 | −1.44049500 | −0.34157400 |
| H | −3.48348300 | −2.26913500 | −0.11094700 |
| H | 2.79075500 | 2.04513800 | −1.74250900 |
| H | 0.62982000 | −1.22221600 | −1.18655800 |
| N | 2.41742400 | −0.83432000 | −1.46295900 |
| O | 1.44756200 | −1.79935400 | −1.47473900 |
| C | 3.47144200 | −1.19809700 | −0.51322200 |
| H | 3.92131800 | −2.15563200 | −0.80619600 |
| H | 4.24746200 | −0.42734600 | −0.55422400 |
| C | 2.93441300 | −1.31346300 | 0.90253300 |
| H | 2.52666000 | −0.34840400 | 1.24427300 |
| H | 2.11679300 | −2.04740200 | 0.92844900 |
| O | 4.01454200 | −1.71637800 | 1.70730600 |
| C | 3.63642400 | −1.88090200 | 3.04934600 |
| H | 2.85446300 | −2.64648000 | 3.15521500 |
| H | 4.52117700 | −2.19552400 | 3.60474300 |
| H | 3.25890500 | −0.94111800 | 3.47857800 |
| C | −5.45944900 | −1.44338100 | −0.28324800 |
| H | −5.80420800 | −2.42506100 | −0.61881000 |
| H | −5.81279000 | −0.70236200 | −1.00413600 |
| C | −5.99988700 | −1.11590100 | 1.10541800 |
| H | −5.65363800 | −0.12432900 | 1.40606700 |
| H | −7.09317600 | −1.11608800 | 1.10624700 |
| H | −5.65457700 | −1.84661300 | 1.84213500 |
| C | 2.90724500 | −0.63343200 | −2.82343300 |
| H | 2.06115000 | −0.32263400 | −3.43739500 |
| H | 3.67269200 | 0.14596100 | −2.82178400 |
| H | 3.32898100 | −1.56311400 | −3.22064800 |

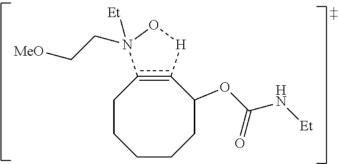

| | | | |
|---|---|---|---|
| C | −0.56699100 | 3.34530000 | 0.10484300 |
| C | 0.95487600 | 3.09126900 | 0.09244400 |
| C | −1.19727800 | 0.55975400 | 0.94949700 |
| C | 1.48009000 | 1.74320300 | −0.41647900 |
| C | −0.04885700 | 0.13149800 | 0.71001400 |
| C | 1.34348000 | 0.56891900 | 0.55720400 |
| H | −1.02615500 | 2.84934900 | −0.76141500 |
| H | 1.36268000 | 3.28112400 | 1.09437800 |
| H | 0.98419000 | 1.47107000 | −1.35633400 |
| H | −0.71042200 | 4.41836500 | −0.05733200 |
| H | 1.40386400 | 3.85318200 | −0.55380600 |
| H | 2.54919100 | 1.85522300 | −0.62264400 |
| H | 1.77304500 | 0.85035300 | 1.52518500 |
| C | −1.33437100 | 2.96207300 | 1.38738900 |
| H | −2.03798000 | 3.75931100 | 1.64925500 |
| H | −0.63359000 | 2.87694000 | 2.22602400 |
| C | −2.12944400 | 1.65351000 | 1.27839600 |
| H | −2.89766900 | 1.75272800 | 0.49853900 |
| 0 | 2.11931200 | −0.54675500 | 0.05292500 |
| C | 3.44195700 | −0.49686500 | 0.31004700 |
| 0 | 4.00149600 | 0.40517100 | 0.90459000 |
| N | 4.06480300 | −1.60483800 | −0.17152000 |
| H | 3.52172900 | −2.21974800 | −0.75708500 |
| H | −2.65547000 | 1.45741200 | 2.22020400 |
| H | −0.60569800 | −1.42363800 | 0.45484400 |
| N | −2.40136600 | −1.06540200 | 0.72778200 |
| O | −1.46316600 | −2.01102000 | 0.41369700 |
| C | −3.34380500 | −0.91345000 | −0.38423100 |
| H | −3.80357600 | −1.87832600 | −0.62814700 |
| H | −4.13388500 | −0.22383500 | −0.06937400 |
| C | −3.02074000 | −1.39666800 | 2.01881100 |
| H | −2.20442900 | −1.37036900 | 2.74613900 |
| C | −3.71822300 | −2.75084400 | 2.03783600 |
| H | −4.59678900 | −2.76425600 | 1.38774300 |
| H | −3.02394100 | −3.52565500 | 1.70787000 |
| H | −4.04991400 | −2.98431400 | 3.05233000 |
| C | −2.65475900 | −0.38611800 | −1.63174700 |
| H | −2.28294500 | 0.63909100 | −1.47619700 |
| H | −1.78838400 | −1.02099000 | −1.86696800 |
| O | −3.61236000 | −0.41912000 | −2.66078200 |
| C | −3.08730300 | 0.03480100 | −3.88095500 |
| H | −2.24140600 | −0.58581700 | −4.20982900 |
| H | −3.88330500 | −0.02420800 | −4.62473700 |
| H | −2.74191100 | 1.07666100 | −3.80744900 |
| C | 5.51429000 | −1.68601800 | −0.15689900 |
| H | 5.78967500 | −2.73946400 | −0.25458700 |
| H | 5.84681400 | −1.34498100 | 0.82635100 |
| C | 6.16833400 | −0.84309900 | −1.24755500 |
| H | 5.88865400 | 0.20455000 | −1.11434100 |
| H | 7.25769800 | −0.92012300 | −1.19665100 |
| H | 5.84580200 | −1.16907300 | −2.24034800 |
| H | −3.71972300 | −0.58868300 | 2.25943100 |

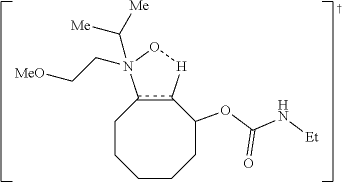

| | | | |
|---|---|---|---|
| C | −0.41285900 | −3.18809300 | −1.24167100 |
| C | 1.09850300 | −2.88234700 | −1.19159900 |
| C | −1.16069400 | −0.31189400 | −0.94820300 |
| C | 1.60892000 | −1.79543200 | −0.23731500 |
| C | −0.01023400 | 0.05826900 | −0.62887100 |
| C | 1.39673700 | −0.35234500 | −0.70446200 |
| H | −0.83863700 | −3.08609900 | −0.23386200 |
| H | 1.45914900 | −2.66763100 | −2.20650600 |
| H | 1.14879600 | −1.90940900 | 0.75212600 |
| H | −0.51223400 | −4.24751500 | −1.49891000 |
| H | 1.60336700 | −3.80740200 | −0.89296900 |
| H | 2.68883600 | −1.93110300 | −0.12159300 |
| H | 1.78149500 | −0.24301600 | −1.72468300 |
| C | −1.26396900 | −2.38461700 | −2.24683500 |
| H | −1.96314600 | −3.05562600 | −2.75684800 |
| H | −0.61684300 | −1.96016700 | −3.02332600 |
| C | −2.08155600 | −1.24954400 | −1.61548600 |
| H | −2.79846500 | −1.66674900 | −0.89403500 |
| O | 2.17416200 | 0.53050300 | 0.14264900 |
| C | 3.48260800 | 0.62824200 | −0.16770500 |
| O | 4.02932200 | 0.02798600 | −1.07384000 |
| N | 4.10659900 | 1.50610900 | 0.66149400 |
| H | 3.58194700 | 1.84263500 | 1.45359100 |
| H | −2.67041000 | −0.73774700 | −2.38743500 |
| H | −0.59357000 | 1.40828100 | 0.16165300 |
| N | −2.39442500 | 1.04512200 | −0.07794300 |
| O | −1.46590200 | 1.89341300 | 0.46082800 |
| C | −3.15731200 | 0.41233900 | 1.00564900 |
| H | −3.57807100 | 1.18042700 | 1.66362400 |
| H | −3.98302800 | −0.15218200 | 0.55995000 |
| C | −3.23637200 | 1.76458200 | −1.06723400 |
| H | −3.91399400 | 1.00172700 | −1.47148400 |
| C | −4.05264200 | 2.87867700 | −0.41251600 |
| H | −4.60152100 | 3.43339900 | −1.17765300 |
| H | −4.77976400 | 2.49363600 | 0.30604600 |
| H | −3.37814300 | 3.56736500 | 0.10273700 |
| C | −2.35993400 | 2.31337200 | −2.18521300 |
| H | −1.65691400 | 3.04285200 | −1.77747600 |
| H | −1.78617900 | 1.52340000 | −2.67360300 |
| H | −2.98675600 | 2.80785000 | −2.93115300 |
| C | −2.29248900 | −0.50218000 | 1.85840300 |
| H | −1.98437200 | −1.40022600 | 1.30083400 |
| H | −1.37866600 | 0.03094600 | 2.15914200 |
| O | −3.07330300 | −0.85546300 | 2.97366700 |
| C | −2.38093800 | −1.70643000 | 3.84927600 |
| H | −1.46729700 | −1.23018500 | 4.23320200 |
| H | −3.04491700 | −1.93036600 | 4.68555000 |
| H | −2.09684700 | −2.64743600 | 3.35516700 |
| C | 5.55149000 | 1.64173100 | 0.61939400 |
| H | 5.81001100 | 2.59562100 | 1.08667400 |
| H | 5.83992400 | 1.69773300 | −0.43290100 |
| C | 6.27778900 | 0.48312100 | 1.29609100 |
| H | 6.01272900 | −0.45259900 | 0.79839300 |
| H | 7.36130300 | 0.61442000 | 1.23281500 |
| H | 6.00049100 | 0.41033100 | 2.35143800 |

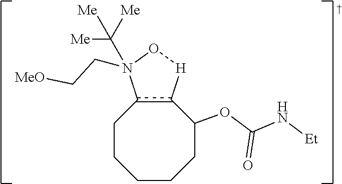

| | | | |
|---|---|---|---|
| C | −0.17559100 | 2.88889100 | 1.76677400 |
| C | 1.33196200 | 2.67089700 | 1.54507600 |
| C | −0.99588100 | 0.11067100 | 0.94020000 |
| C | 1.78125800 | 1.72697400 | 0.42549500 |
| C | 0.14836300 | −0.15943000 | 0.49492300 |
| C | 1.55353100 | 0.23251600 | 0.68016800 |
| H | −0.67976500 | 2.95723800 | 0.79305900 |
| H | 1.79343600 | 2.35107400 | 2.48890100 |
| H | 1.29180900 | 1.99541100 | −0.51907600 |
| H | −0.29140900 | 3.87390500 | 2.23020600 |
| H | 1.77393900 | 3.64715400 | 1.31715800 |
| H | 2.85922400 | 1.86152700 | 0.29302000 |
| H | 1.91374800 | −0.02368700 | 1.68284900 |
| C | −0.89961100 | 1.85778800 | 2.65691500 |
| H | −1.51272600 | 2.37604300 | 3.40162500 |
| H | −0.16378600 | 1.26922700 | 3.21755000 |
| C | −1.81382700 | 0.89009100 | 1.89215000 |
| H | −2.59788300 | 1.45590000 | 1.36901700 |
| O | 2.35461700 | −0.52339700 | −0.26401500 |
| C | 3.65553500 | −0.66084700 | 0.05922600 |
| O | 4.18008300 | −0.18995700 | 1.05117100 |
| N | 4.30190900 | −1.41646900 | −0.86833000 |
| H | 3.79759800 | −1.63509800 | −1.71330300 |
| H | −2.31884500 | 0.23071300 | 2.60749900 |
| H | −0.41749700 | −1.11096700 | −0.63065800 |
| N | −2.23048000 | −0.91825200 | −0.24983000 |
| O | −1.30687300 | −1.49509400 | −1.07354900 |
| C | −3.04053800 | 0.03411400 | −1.02972700 |
| H | −3.60233000 | −0.49787600 | −1.80615600 |
| H | −3.75345500 | 0.52097200 | −0.36138800 |
| C | −3.01890400 | −2.01871800 | 0.42399500 |
| C | −3.46875600 | −3.04523300 | −0.62311600 |
| H | −4.07366400 | −3.81255300 | −0.13127700 |
| H | −4.08420800 | −2.58535800 | −1.40132100 |
| H | −2.60639300 | −3.51679100 | −1.09357600 |
| C | −2.09164100 | −2.69616700 | 1.43236400 |
| H | −1.15265700 | −2.97386900 | 0.94970600 |
| H | −1.86588300 | −2.03996500 | 2.27653300 |
| H | −2.57298900 | −3.60033900 | 1.81511900 |
| C | −2.19006800 | 1.09641900 | −1.70757500 |
| H | −1.71113200 | 1.75142400 | −0.96394100 |
| H | −1.39452000 | 0.62234000 | −2.29849500 |
| O | −3.06894800 | 1.83279300 | −2.52376400 |
| C | −2.40675800 | 2.86581000 | −3.20544300 |
| H | −1.61468600 | 2.47289000 | −3.85901800 |
| H | −3.14678800 | 3.38634000 | −3.81517100 |
| H | −1.95122400 | 3.58189300 | −2.50526600 |
| C | 5.74674900 | −1.54820500 | −0.81567700 |
| H | 6.02251400 | −2.42859100 | −1.40221800 |
| H | 6.01468700 | −1.74455800 | 0.22511000 |
| C | 6.47679900 | −0.30457700 | −1.31400000 |
| H | 6.19506500 | 0.55336300 | −0.69910100 |
| H | 7.55992800 | −0.43762800 | −1.24760900 |
| H | 6.21920900 | −0.09017800 | −2.35499100 |
| C | −4.25498400 | −1.45277800 | 1.12819300 |
| H | −4.68632500 | −2.24262000 | 1.74887600 |
| H | −4.01749500 | −0.60950600 | 1.77932100 |
| H | −5.02236700 | −1.13884200 | 0.41574800 |

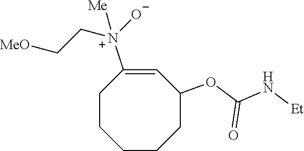 [SMILES: CCNC(=O)OC1/C=C(/[N+](C)([O-])CCOC)CCCCC1]

| | | | |
|---|---|---|---|
| C | 0.01529800 | 3.11130800 | 1.27132100 |
| C | −1.21997000 | 2.23834200 | 1.49833800 |
| C | 1.41096700 | 0.50295700 | −0.76681100 |
| C | −1.02003900 | 0.74430900 | 1.24314700 |
| C | 0.23306800 | −0.10102400 | −0.83562700 |
| C | −1.06465200 | 0.37350100 | −0.25564200 |
| H | 0.73723300 | 2.93511900 | 2.08015600 |
| H | −2.04742200 | 2.57415900 | 0.86241400 |
| H | −0.08153900 | 0.39167500 | 1.68905000 |
| H | −0.29135700 | 4.15861200 | 1.36591800 |
| H | −1.55005200 | 2.38329800 | 2.53249400 |
| H | −1.82814600 | 0.19442200 | 1.73207800 |
| H | −1.45658400 | 1.22909300 | −0.81741700 |
| C | 0.75022500 | 2.93610300 | −0.06706100 |
| H | 1.28685800 | 3.86822100 | −0.26725200 |
| H | 0.03908400 | 2.82424500 | −0.89298500 |
| C | 1.80087900 | 1.80182800 | −0.10496400 |
| H | 2.13951100 | 1.59099300 | 0.92071100 |
| O | −1.98308900 | −0.71447300 | −0.47445200 |
| C | −3.28761900 | −0.37368700 | −0.39503700 |
| O | −3.69027300 | 0.74539000 | −0.13805400 |
| N | −4.07945500 | −1.44326700 | −0.65466200 |
| H | −3.63032000 | −2.34119800 | −0.74447500 |
| H | 2.68585700 | 2.19251400 | −0.61464200 |
| H | 0.24228300 | −1.05380500 | −1.36022500 |
| N | 2.54108200 | −0.26389400 | −1.43836800 |
| O | 2.16993000 | −1.49406200 | −1.86344200 |
| C | 3.68554300 | −0.42759000 | −0.48098900 |
| H | 4.48488100 | −0.89402800 | −1.05823100 |
| H | 4.02527100 | 0.54054100 | −0.10369000 |
| C | 3.28543900 | −1.33696300 | 0.66479600 |
| H | 2.39471900 | −0.94386500 | 1.18365100 |
| H | 3.03221200 | −2.32388800 | 0.26200400 |
| O | 4.39042900 | −1.38170200 | 1.53554500 |
| C | 4.17766800 | −2.26779400 | 2.60355800 |
| H | 4.00413000 | −3.29269800 | 2.24638100 |
| H | 5.07265500 | −2.25319000 | 3.22746100 |
| H | 3.31247600 | −1.96388800 | 3.21129600 |
| C | −5.51787600 | −1.34502700 | −0.47800200 |
| H | −5.97835600 | −2.15286700 | −1.05242600 |
| H | −5.83485000 | −0.39837000 | −0.92199900 |
| C | −5.94021900 | −1.40065900 | 0.98676200 |
| H | −5.47764500 | −0.57413700 | 1.53123600 |
| H | −7.02576100 | −1.31202900 | 1.08014100 |
| H | −5.63009100 | −2.34235000 | 1.44786700 |
| C | 2.99006200 | 0.49954100 | −2.64389200 |
| H | 3.76116100 | −0.11025800 | −3.11116400 |
| H | 2.12144500 | 0.55980500 | −3.29669400 |
| H | 3.37232900 | 1.49191100 | −2.39661100 |

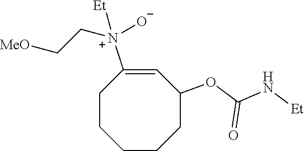 [SMILES: CCNC(=O)OC1/C=C(/[N+]([O-])(CC)CCOC)CCCCC1]

| | | | |
|---|---|---|---|
| C | −0.32359600 | 3.10280900 | 1.49767000 |
| C | −1.56323600 | 2.21066700 | 1.58875300 |
| C | 1.27084400 | 0.53740000 | −0.47421600 |
| C | −1.32238500 | 0.72542200 | 1.31409200 |
| C | 0.10445700 | −0.05829200 | −0.68072300 |
| C | −1.23944600 | 0.39098600 | −0.19184800 |
| H | 0.31415300 | 2.92903200 | 2.37488100 |
| H | −2.33314100 | 2.55387400 | 0.88797400 |
| H | −0.42105900 | 0.36979500 | 1.82889500 |
| H | −0.65193800 | 4.14548600 | 1.56722300 |
| H | −1.98935100 | 2.32492200 | 2.59110600 |
| H | −2.16149300 | 0.15485600 | 1.71996000 |
| H | −1.59263000 | 1.25730700 | −0.76340000 |
| C | 0.54654000 | 2.94357400 | 0.24174600 |
| H | 1.10744800 | 3.87388200 | 0.11214200 |
| H | −0.07569600 | 2.84851500 | −0.65499100 |
| C | 1.58260200 | 1.79524800 | 0.30067100 |
| H | 1.76694500 | 1.52828300 | 1.35205100 |
| O | −2.12709800 | −0.69813000 | −0.51059900 |
| C | −3.43688600 | −0.36898100 | −0.53039000 |
| O | −3.86965400 | 0.74078600 | −0.28218800 |
| N | −4.19504100 | −1.43821700 | −0.87730000 |
| H | −3.73176900 | −2.33049000 | −0.94989200 |
| H | 2.53603300 | 2.19375600 | −0.05366900 |
| H | 0.15658500 | −0.97734100 | −1.25922600 |
| N | 2.46059800 | −0.18779900 | −1.08391100 |
| O | 2.12945900 | −1.38930200 | −1.60771800 |
| C | 3.49983600 | −0.40998000 | −0.02136900 |
| H | 4.32867100 | −0.91525900 | −0.51489200 |
| H | 3.84531100 | 0.54040400 | 0.39476700 |
| C | 2.99622500 | 0.67749600 | −2.21098900 |
| H | 2.13838100 | 0.75572000 | −2.88022300 |
| C | 4.16978300 | 0.02500400 | −2.91513000 |
| H | 5.09442900 | 0.09366700 | −2.33709600 |
| H | 3.92103200 | −1.02128200 | −3.09506300 |
| H | 4.33748900 | 0.52869600 | −3.86979300 |
| C | 2.95722400 | −1.31823600 | 1.06595800 |
| H | 2.11138800 | −0.84862200 | 1.59572700 |
| H | 2.59626300 | −2.24083100 | 0.59619500 |
| O | 4.02548100 | −1.55134900 | 1.95199600 |
| C | 3.66857100 | −2.44118100 | 2.97752700 |
| H | 3.35857200 | −3.41534600 | 2.57375200 |
| H | 4.54443200 | −2.57983400 | 3.61319200 |
| H | 2.84373000 | −2.04254200 | 3.58674700 |
| C | −5.64394000 | −1.35484900 | −0.81520700 |
| H | −6.04888800 | −2.15597000 | −1.43877300 |
| H | −5.93357000 | −0.40296500 | −1.26669200 |
| C | −6.18192500 | −1.44052900 | 0.60964500 |
| H | −5.77265000 | −0.62012300 | 1.20379800 |
| H | −7.27228800 | −1.36269200 | 0.61681900 |
| H | −5.90063700 | −2.38784000 | 1.07771100 |
| H | 3.25182100 | 1.66967600 | −1.82983600 |

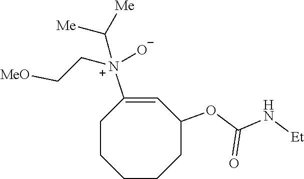 [SMILES: CCNC(=O)OC1/C=C(/[N+]([O-])(CCOC)C(C)C)CCCCC1]

| | | | |
|---|---|---|---|
| C | 0.43248800 | 1.70511700 | −3.05767900 |
| C | 1.68494200 | 0.96122000 | −2.58923600 |
| C | −1.23799000 | 0.57214500 | −0.05801300 |
| C | 1.44766500 | −0.13983500 | −1.55345500 |
| C | −0.07556700 | 0.25028100 | 0.49288000 |
| C | 1.28694900 | 0.40002400 | −0.11498100 |
| H | −0.14524000 | 1.05749300 | −3.73081900 |
| H | 2.40767700 | 1.66456500 | −2.15951600 |
| H | 0.58282600 | −0.75848200 | −1.82346000 |
| H | 0.75265200 | 2.55935200 | −3.66388400 |
| H | 2.16949700 | 0.52568300 | −3.46948300 |
| H | 2.31649500 | −0.80262700 | −1.54679800 |
| H | 1.60405500 | 1.44971800 | −0.09713000 |
| C | −0.51399600 | 2.20634400 | −1.95706000 |
| H | −1.10265500 | 3.02801900 | −2.37549000 |
| H | 0.05393500 | 2.64384500 | −1.12783000 |
| C | −1.51311900 | 1.14957300 | −1.42539800 |
| H | −1.58976000 | 0.32610600 | −2.15087400 |
| O | 2.16564300 | −0.30670100 | 0.78224400 |
| C | 3.46975500 | 0.02735800 | 0.67138400 |
| O | 3.90601600 | 0.83513600 | −0.12710900 |
| N | 4.21703400 | −0.64912700 | 1.57806400 |
| H | 3.75761400 | −1.36930300 | 2.11303400 |
| H | −2.50541200 | 1.60514200 | −1.41924400 |
| H | −0.14153400 | −0.16276000 | 1.49609600 |
| N | −2.45550000 | 0.25914500 | 0.79730200 |
| O | −2.12621200 | −0.32397100 | 1.96937600 |
| C | −3.32672700 | −0.69332500 | 0.02185000 |
| H | −4.15656700 | −0.94200800 | 0.68026400 |
| H | −3.70457600 | −0.21537900 | −0.88702800 |
| C | −3.22621400 | 1.56491600 | 1.09566000 |
| H | −3.59538300 | 1.95387800 | 0.14194000 |
| C | −4.39970400 | 1.25671500 | 2.01425800 |
| H | −4.83919300 | 2.19805200 | 2.35224800 |
| H | −5.18747700 | 0.68776400 | 1.51635700 |
| H | −4.03023200 | 0.69152700 | 2.87070100 |
| C | −2.25888300 | 2.54223000 | 1.74426900 |
| H | −1.75365600 | 2.02276500 | 2.56196000 |
| H | −1.50986000 | 2.91362200 | 1.04121900 |
| H | −2.81165300 | 3.39644300 | 2.14087700 |
| C | −2.57798700 | −1.97555300 | −0.29144600 |
| H | −1.79746200 | −1.81602000 | −1.05355000 |
| H | −2.09606600 | −2.32147400 | 0.63196100 |
| O | −3.54140400 | −2.88692500 | −0.76268300 |
| C | −2.97847600 | −4.14103700 | −1.04731000 |
| H | −2.51506700 | −4.58452800 | −0.15469600 |
| H | −3.78256800 | −4.79210400 | −1.39366600 |
| H | −2.21240200 | −4.06905300 | −1.83373400 |
| C | 5.66626800 | −0.55716500 | 1.54264000 |
| H | 6.04205800 | −0.87007300 | 2.52011600 |
| H | 5.92139300 | 0.49694300 | 1.40990000 |
| C | 6.28539200 | −1.38786900 | 0.42299100 |
| H | 5.90333200 | −1.04309700 | −0.54072000 |
| H | 7.37364100 | −1.28439200 | 0.42094900 |
| H | 6.03972100 | −2.44689500 | 0.53997800 |

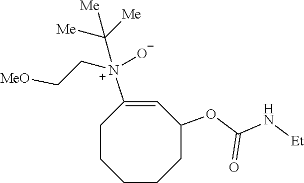 [SMILES: CCNC(=O)OC1/C=C(/[N+]([O-])(CCOC)C(C)(C)C)CCCCC1]

| | | | |
|---|---|---|---|
| C | 0.53785400 | 0.06577100 | 3.47621400 |
| C | 1.82047800 | 0.45628600 | 2.73509500 |
| C | −1.07343800 | −0.27912400 | 0.22696900 |
| C | 1.64475000 | 0.96075600 | 1.29891800 |
| C | 0.10935700 | −0.29753500 | −0.37569500 |
| C | 1.45917000 | −0.17582200 | 0.26902600 |
| H | 0.00561800 | 0.97322900 | 3.79088500 |
| H | 2.50756800 | −0.39707900 | 2.69387700 |
| H | 0.82651600 | 1.68581900 | 1.22024100 |
| H | 0.82280600 | −0.45179200 | 4.39851400 |
| H | 2.32974400 | 1.22748000 | 3.32257200 |
| H | 2.55621700 | 1.49146400 | 1.01344000 |
| H | 1.73477800 | −1.12442700 | 0.74689800 |
| C | −0.44781700 | −0.80977800 | 2.69131600 |
| H | −1.10886600 | −1.30533100 | 3.40986800 |
| H | 0.08501500 | −1.61619300 | 2.17346600 |
| C | −1.33148400 | −0.01753400 | 1.69398700 |
| H | −1.20525400 | 1.05808400 | 1.87643800 |
| O | 2.37125700 | 0.00387400 | −0.83276400 |
| C | 3.65819000 | −0.29574700 | −0.55279200 |
| O | 4.05577500 | −0.66754800 | 0.53540500 |
| N | 4.43714100 | −0.13878700 | −1.65158400 |
| H | 4.01285600 | 0.28165400 | −2.46352900 |
| H | −2.37372800 | −0.21498900 | 1.93163600 |
| H | 0.08279300 | −0.43664800 | −1.45267500 |
| N | −2.26363600 | −0.45883500 | −0.70448000 |
| O | −1.90539900 | −0.31206700 | −2.00384000 |
| C | −3.27343200 | 0.61711500 | −0.41830000 |
| H | −4.11553000 | 0.43300600 | −1.08108700 |
| H | −3.61679300 | 0.59198300 | 0.61719100 |
| C | −2.87690900 | −1.90547800 | −0.54583800 |
| C | −4.06972100 | −1.99205500 | −1.49930300 |
| H | −4.31465300 | −3.04542800 | −1.65683800 |
| H | −4.96220000 | −1.50799000 | −1.09301100 |
| H | −3.79574000 | −1.53749100 | −2.45185400 |
| C | −1.79550400 | −2.88361300 | −0.99644800 |
| H | −1.44704100 | −2.60231900 | −1.99001500 |
| H | −0.94849300 | −2.89454400 | −0.30657100 |
| H | −2.22428400 | −3.88872000 | −1.02493400 |
| C | −2.69290300 | 1.98463000 | −0.73886700 |
| H | −1.74214700 | 2.15132100 | −0.20655700 |
| H | −2.48995500 | 2.04480100 | −1.81341500 |
| O | −3.66263100 | 2.92035500 | −0.32880200 |
| C | −3.29035300 | 4.23400700 | −0.65388700 |
| H | −3.15786900 | 4.35773800 | −1.73811500 |
| H | −4.08730300 | 4.89695900 | −0.31330000 |
| H | −2.35074600 | 4.51988700 | −0.15801400 |
| C | 5.88077200 | −0.25866800 | −1.54413300 |
| H | 6.27362500 | −0.44468000 | −2.54698900 |
| H | 6.08792600 | −1.14260600 | −0.93624100 |
| C | 6.53241200 | 0.96946500 | −0.91640600 |
| H | 6.13215800 | 1.12107700 | 0.08879000 |
| H | 7.61498000 | 0.83737300 | −0.84155200 |
| H | 6.33457600 | 1.86479900 | −1.51210600 |
| C | −3.32401700 | −2.24284400 | 0.87598700 |
| H | −3.83257300 | −3.21080800 | 0.83642800 |
| H | −2.48351200 | −2.34721900 | 1.56636100 |
| H | −4.04147300 | −1.52095200 | 1.27759400 |

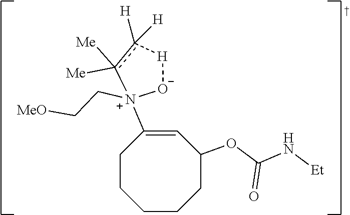

| | | | |
|---|---|---|---|
| C | 0.29490300 | 1.26618700 | 3.13720900 |
| C | 1.63867300 | 1.24411700 | 2.40455900 |
| C | −1.23953700 | −0.02534900 | 0.10160000 |
| C | 1.54052200 | 1.19399000 | 0.87812500 |
| C | −0.04941800 | −0.41970600 | −0.35078000 |
| C | 1.27538700 | −0.22650600 | 0.32977100 |
| H | −0.14927400 | 2.26678800 | 3.05316000 |
| H | 2.23451000 | 0.38156300 | 2.72469500 |
| H | 0.77000000 | 1.88171800 | 0.50933500 |
| H | 0.48374400 | 1.10706100 | 4.20451800 |
| H | 2.20500800 | 2.13349500 | 2.70082800 |
| H | 2.49220900 | 1.52547800 | 0.45405300 |
| H | 1.41996500 | −0.95101100 | 1.14016400 |
| C | −0.74763400 | 0.24682400 | 2.65595100 |
| H | −1.46707300 | 0.10148200 | 3.46861900 |
| H | −0.28375600 | −0.73373400 | 2.49759600 |
| C | −1.54532300 | 0.68123500 | 1.40255700 |
| H | −1.43739100 | 1.76037300 | 1.23972600 |
| O | 2.24884700 | −0.55453800 | −0.68496700 |
| C | 3.48374800 | −0.82965100 | −0.21587700 |
| O | 3.79961100 | −0.79256400 | 0.95827600 |
| N | 4.31856700 | −1.16800500 | −1.23168600 |
| H | 3.97573800 | −1.04480900 | −2.17147400 |
| H | −2.60673500 | 0.52671300 | 1.60894600 |
| H | −0.02534900 | −0.91610400 | −1.31380200 |
| N | −2.40811100 | −0.27421400 | −0.73740900 |
| O | −2.11546500 | −0.86057700 | −1.90090200 |
| C | −3.18696200 | 0.95932400 | −1.02829000 |
| H | −4.04919400 | 0.62280600 | −1.60825800 |
| H | −3.53494700 | 1.43011600 | −0.10603700 |
| C | −3.66474600 | −1.73000000 | 0.10231000 |
| C | −3.86654100 | −2.46649100 | −1.10202300 |
| H | −3.49344800 | −3.48982600 | −1.10104700 |
| H | −4.84058800 | −2.35827600 | −1.57730700 |
| H | −2.99808700 | −1.82961600 | −1.79461200 |
| C | −2.73252200 | −2.34666000 | 1.11374700 |
| H | −1.79632200 | −2.65086300 | 0.63729000 |
| H | −2.50191100 | −1.67978400 | 1.94744700 |
| H | −3.21114900 | −3.24231800 | 1.52618500 |
| C | −2.37098100 | 1.94566200 | −1.84489500 |
| H | −1.78414500 | 1.37093800 | −2.57357800 |
| H | −3.03998800 | 2.62748000 | −2.39210300 |
| O | −1.53097600 | 2.68556500 | −0.98372400 |
| C | −0.42830700 | 3.23250500 | −1.66277300 |
| H | −0.74530400 | 3.86755000 | −2.50242700 |
| H | 0.12833900 | 3.84339300 | −0.94937300 |
| H | 0.22880500 | 2.43819500 | −2.04651500 |
| C | 5.73722000 | −1.34682700 | −0.97750200 |
| H | 6.15130400 | −1.93251900 | −1.80235500 |
| H | 5.82958100 | −1.94317500 | −0.06673300 |
| C | 6.48188700 | −0.02570300 | −0.81200700 |
| H | 6.05695200 | 0.52916300 | 0.02771000 |
| H | 7.54188700 | −0.20109900 | −0.61023400 |
| H | 6.39947000 | 0.58572200 | −1.71488000 |
| C | −4.81330200 | −0.93450600 | 0.66756600 |
| H | −5.47143600 | −1.62990200 | 1.20309600 |
| H | −4.50309700 | −0.16954100 | 1.38431100 |
| H | −5.41004900 | −0.46567700 | −0.11821900 |

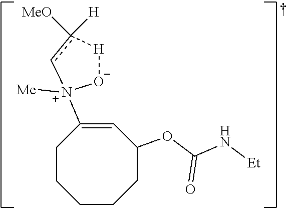

| | | | |
|---|---|---|---|
| C | 0.78474600 | 3.57538900 | −0.31234800 |
| C | 1.96343300 | 2.64461000 | −0.61298600 |
| C | −1.21205600 | 0.59449300 | 0.28724000 |
| C | 1.60993500 | 1.20552300 | −1.00493400 |
| C | −0.11506800 | −0.15203300 | 0.39660700 |
| C | 1.30733800 | 0.29364500 | 0.20396200 |
| H | 0.30355100 | 3.87170300 | −1.25402700 |
| H | 2.63060000 | 2.58864900 | 0.25536100 |
| H | 0.78608900 | 1.17066800 | −1.72624900 |
| H | 1.18340100 | 4.49698200 | 0.12520400 |
| H | 2.55325800 | 3.09549000 | −1.41794100 |
| H | 2.47841000 | 0.77222000 | −1.50666200 |
| H | 1.66877700 | 0.79320300 | 1.11183600 |
| C | −0.29833000 | 3.00624600 | 0.61280400 |
| H | −0.86759900 | 3.84363400 | 1.02713500 |
| H | 0.15915600 | 2.50214900 | 1.47199100 |
| C | −1.28923200 | 2.05399900 | −0.09892500 |
| H | −1.14800000 | 2.13432900 | −1.18407700 |
| O | 2.05428700 | −0.93506700 | 0.09139800 |
| C | 3.37397700 | −0.81833600 | 0.35812600 |
| O | 3.92525100 | 0.22528300 | 0.65206600 |
| N | 3.98973300 | −2.02342200 | 0.26691700 |
| H | 3.45100800 | −2.79053700 | −0.10381500 |
| H | −2.31048300 | 2.40411100 | 0.06765000 |
| H | −0.24798600 | −1.18972800 | 0.68096800 |
| N | −2.50432400 | −0.03613700 | 0.51482000 |
| O | −2.46289800 | −1.36810700 | 0.60236200 |
| C | −3.63032200 | 0.21681000 | −1.07065400 |
| H | −4.23319500 | 1.02886700 | −0.66978900 |
| H | −2.86935500 | 0.50785600 | −1.78942600 |
| C | −4.18507300 | −1.06769100 | −1.18956900 |
| H | −4.07397900 | −1.59543000 | −2.14033300 |
| H | −3.23155100 | −1.58537900 | −0.32531900 |
| O | −5.44529900 | −1.20983900 | −0.57897500 |
| C | −5.64251900 | −2.50272300 | −0.06071000 |
| H | −4.88852800 | −2.74551200 | 0.70049900 |
| H | −6.63681300 | −2.52690600 | 0.39053400 |
| H | −5.59662200 | −3.26609400 | −0.85101600 |
| C | 5.43724200 | −2.10913300 | 0.35649500 |
| H | 5.69342200 | −3.14187100 | 0.60650200 |
| H | 5.74704900 | −1.47764500 | 1.19245500 |
| C | 6.13827600 | −1.66177900 | −0.92231000 |
| H | 5.87701300 | −0.62301200 | −1.13748300 |
| H | 7.22375200 | −1.72981800 | −0.81250400 |
| H | 5.83815200 | −2.28257400 | −1.77095800 |
| C | −3.26030100 | 0.49421000 | 1.66358900 |
| H | −4.25308500 | 0.04440000 | 1.61682500 |
| H | −2.75251600 | 0.19901000 | 2.58469600 |
| H | −3.34479400 | 1.58059500 | 1.61266500 |

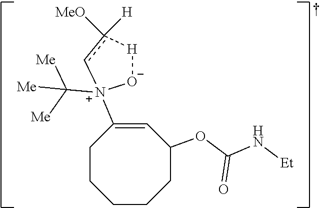

| | | | |
|---|---|---|---|
| C | 1.18481300 | 3.49638700 | −0.85387100 |
| C | 2.39585800 | 2.56684200 | −0.98461800 |
| C | −0.80680200 | 0.53307600 | −0.08690500 |
| C | 2.10477300 | 1.08750100 | −1.26624200 |
| C | 0.29968800 | −0.17053300 | 0.15012800 |
| C | 1.72606100 | 0.27982900 | −0.00589400 |
| H | 0.76947000 | 3.69993900 | −1.84983200 |
| H | 2.99863900 | 2.60625500 | −0.06934600 |
| H | 1.35136900 | 0.95737700 | −2.05015100 |
| H | 1.53746100 | 4.46017000 | −0.47116400 |
| H | 3.03567300 | 2.95693500 | −1.78314400 |
| H | 3.02498700 | 0.63889400 | −1.64771400 |
| H | 2.02520400 | 0.86125000 | 0.87601000 |
| C | 0.04686100 | 2.98251800 | 0.03612100 |
| H | −0.57673700 | 3.83526500 | 0.32414000 |
| H | 0.45128000 | 2.57682300 | 0.97185900 |
| C | −0.85032700 | 1.93194600 | −0.66251400 |
| H | −0.56514700 | 1.87436400 | −1.71991500 |
| O | 2.49136400 | −0.94268400 | 0.03620900 |
| C | 3.78678400 | −0.78473300 | 0.38735800 |
| O | 4.30455000 | 0.28697000 | 0.63811900 |
| N | 4.42015000 | −1.98328400 | 0.43500300 |
| H | 3.91864800 | −2.78477900 | 0.08532300 |
| H | −1.88260300 | 2.27971700 | −0.67430200 |
| H | 0.17564500 | −1.17491100 | 0.53993400 |
| N | −2.09992500 | −0.11067200 | 0.18060300 |
| O | −2.00648100 | −1.44334600 | 0.21071900 |
| C | −3.20161100 | 0.13189800 | −1.45490100 |
| H | −3.92677100 | 0.85016000 | −1.08396100 |
| H | −2.41987100 | 0.54570400 | −2.08221700 |
| C | −2.80011700 | 0.35115500 | 1.45973100 |
| C | −4.12360400 | −0.40866600 | 1.56054000 |
| H | −4.66558000 | −0.05032700 | 2.44011300 |
| H | −4.75338700 | −0.26754200 | 0.67913800 |
| H | −3.93750200 | −1.47680300 | 1.67622800 |
| C | −1.90948300 | −0.01747900 | 2.64916100 |
| H | −1.66687400 | −1.08126400 | 2.61562300 |
| H | −0.98000900 | 0.55840300 | 2.64789500 |
| H | −2.44417500 | 0.19573900 | 3.57924700 |
| C | −3.60175300 | −1.19272700 | −1.71126300 |
| H | −3.28045600 | −1.64150800 | −2.65579500 |
| H | −2.74318300 | −1.66777200 | −0.76505600 |
| O | −4.93095400 | −1.49178400 | −1.36254400 |
| C | −5.08993100 | −2.83342200 | −0.97446900 |
| H | −4.45932400 | −3.07757700 | −0.10810700 |
| H | −6.14006400 | −2.97766200 | −0.71184900 |
| H | −4.83223500 | −3.52179700 | −1.79255800 |
| C | 5.85828700 | −2.03767600 | 0.63403200 |
| H | 6.10768900 | −3.04528900 | 0.97630400 |
| H | 6.09930000 | −1.34267800 | 1.44181500 |
| C | 6.64498900 | −1.67151100 | −0.62055600 |
| H | 6.38732200 | −0.65564100 | −0.92911900 |
| H | 7.72036400 | −1.71255500 | −0.42864100 |
| H | 6.41486600 | −2.35700300 | −1.44081000 |
| C | −3.06624400 | 1.85701000 | 1.47666600 |
| H | −3.62599800 | 2.08600100 | 2.38752300 |
| H | −2.14362200 | 2.44150800 | 1.50933300 |
| H | −3.67325300 | 2.18656700 | 0.62891200 |

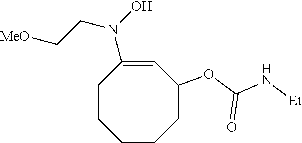 [SMILES: CCNC(=O)OC1/C=C(/N(O)CCOC)CCCCC1]

| | | | |
|---|---|---|---|
| C | 0.07672000 | 3.22876300 | 0.93214200 |
| C | −1.11895800 | 2.33695100 | 1.26886200 |
| C | 1.51825000 | 0.50026400 | −0.96841100 |
| C | −0.87103900 | 0.83861100 | 1.09891400 |
| C | 0.33760700 | −0.12364500 | −0.96724000 |
| C | −0.95006000 | 0.37336300 | −0.37220300 |
| H | 0.83162100 | 3.13687200 | 1.72491000 |
| H | −1.98130100 | 2.60233400 | 0.64619400 |
| H | 0.09676900 | 0.55029700 | 1.52744100 |
| H | −0.26033400 | 4.27083400 | 0.95788000 |
| H | −1.41391200 | 2.53886900 | 2.30425400 |
| H | −1.63809100 | 0.29049800 | 1.65278600 |
| H | −1.38990700 | 1.17900700 | −0.97106300 |
| C | 0.77086100 | 2.96949300 | −0.41406800 |
| H | 1.27460700 | 3.89752400 | −0.70079500 |
| H | 0.03652500 | 2.78111100 | −1.20481200 |
| C | 1.85468000 | 1.86888300 | −0.40963300 |
| H | 2.25146200 | 1.75948100 | 0.61046500 |
| O | −1.85085800 | −0.75033900 | −0.48587700 |
| C | −3.15883400 | −0.44153800 | −0.37126400 |
| O | −3.58950000 | 0.67974100 | −0.17973000 |
| N | −3.92873000 | −1.55170200 | −0.51652900 |
| H | −3.45345800 | −2.44070600 | −0.52428300 |
| H | 2.69337000 | 2.22815900 | −1.01431700 |
| H | 0.27892700 | −1.09974100 | −1.43233300 |
| N | 2.66508600 | −0.07923800 | −1.60976900 |
| O | 2.36766800 | −1.35352700 | −2.14964800 |
| C | 3.81937400 | −0.27254800 | −0.72659800 |
| H | 4.62311400 | −0.70026000 | −1.32943100 |
| H | 4.15642300 | 0.70186800 | −0.36562500 |
| H | 2.36107600 | −1.19120000 | −3.10068000 |
| C | 3.53493100 | −1.18467700 | 0.45931000 |
| H | 2.74524400 | −0.75110400 | 1.09681700 |
| H | 3.17231100 | −2.16047000 | 0.10264200 |
| O | 4.73738400 | −1.31815500 | 1.17553500 |
| C | 4.59855400 | −2.15794800 | 2.29125400 |
| H | 4.29078400 | −3.17157100 | 1.99588300 |
| H | 5.56877500 | −2.21111200 | 2.78781100 |
| H | 3.85445400 | −1.76571100 | 3.00018000 |
| C | −5.35493600 | −1.48234500 | −0.24963300 |
| H | −5.82512500 | −2.34440500 | −0.72999700 |
| H | −5.73100600 | −0.58178600 | −0.74053600 |
| C | −5.67969100 | −1.43717300 | 1.24031800 |
| H | −5.21111900 | −0.55807400 | 1.68894700 |
| H | −6.75929700 | −1.37336300 | 1.39985600 |
| H | −5.30871100 | −2.33125400 | 1.74915800 |

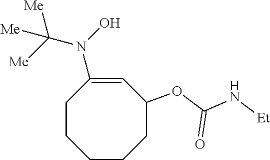 [SMILES: CCNC(=O)OC1/C=C(/N(O)C(C)(C)C)CCCCC1]

| | | | |
|---|---|---|---|
| C | −1.15352700 | −3.47073000 | 0.87783200 |
| C | −2.40816700 | −2.70500300 | 0.44605700 |
| C | 0.80428600 | −0.55606400 | −0.11909300 |
| C | −2.18783400 | −1.53441500 | −0.51801900 |
| C | −0.29233600 | 0.20007300 | −0.07426000 |
| C | −1.70467700 | −0.24614600 | 0.18252700 |
| H | −0.83138900 | −4.13491900 | 0.06485600 |
| H | −2.92545800 | −2.30306000 | 1.32521400 |
| H | −1.51088000 | −1.80104900 | −1.33731100 |
| H | −1.42477900 | −4.12600000 | 1.71249900 |
| H | −3.10121200 | −3.41786600 | −0.01295900 |
| H | −3.14947500 | −1.29400700 | −0.97770000 |
| H | −1.87226000 | −0.35534300 | 1.26168600 |
| C | 0.04727900 | −2.60658600 | 1.28335600 |
| H | 0.72108800 | −3.22211000 | 1.88761900 |
| H | −0.27392800 | −1.78745900 | 1.93695300 |
| C | 0.85491800 | −2.05625300 | 0.07923300 |
| H | 0.51469400 | −2.56000300 | −0.83350200 |
| O | −2.51058000 | 0.87303900 | −0.24189300 |
| C | −3.75287800 | 0.90580100 | 0.28867100 |
| O | −4.19711900 | 0.06734500 | 1.04960800 |
| N | −4.42557100 | 2.00581300 | −0.13266600 |
| H | −3.99652500 | 2.56099400 | −0.85633200 |
| H | 1.89786400 | −2.35017200 | 0.19747400 |
| H | −0.16060600 | 1.27063400 | −0.17935200 |
| N | 2.09722100 | 0.12243000 | −0.28835900 |
| O | 2.00293500 | 1.44118000 | −0.09925600 |
| C | 2.81332700 | −0.11787900 | −1.61825600 |
| C | 3.17362400 | −0.38890000 | 1.30632600 |
| H | 2.38159800 | −0.91157100 | 1.83394500 |
| H | 3.91787300 | −1.02333200 | 0.83428600 |
| C | 4.09467900 | 0.71876700 | −1.59973800 |
| H | 4.71481800 | 0.49743000 | −0.72662500 |
| H | 4.66952700 | 0.49832100 | −2.50338900 |
| H | 3.85534600 | 1.78189600 | −1.58680900 |
| C | 1.89071200 | 0.36827800 | −2.73793000 |
| H | 0.99083200 | −0.24975500 | −2.80944300 |
| H | 1.59226700 | 1.40132800 | −2.54884800 |
| H | 2.42006300 | 0.31880700 | −3.69391700 |
| C | 3.55400000 | 0.87620900 | 1.79092600 |
| H | 3.20985200 | 1.15858900 | 2.79011900 |
| H | 2.71646400 | 1.49908400 | 0.92161700 |
| O | 4.88710700 | 1.23712400 | 1.52405900 |
| C | 5.04021900 | 2.62573300 | 1.36443400 |
| H | 6.09466000 | 2.81817000 | 1.15559300 |
| H | 4.42790800 | 3.00302500 | 0.53348100 |
| H | 4.75554800 | 3.16887500 | 2.27725400 |
| C | −5.83454700 | 2.16216900 | 0.18498100 |
| H | −5.95878400 | 1.90270700 | 1.23894500 |
| H | −6.08496200 | 3.21995000 | 0.07073900 |
| C | −6.73760700 | 1.28585000 | −0.67728200 |
| H | −6.47474800 | 0.23563500 | −0.53045100 |
| H | −7.78659100 | 1.42171700 | −0.40137200 |
| H | −6.62538200 | 1.53134700 | −1.73692800 |
| C | 3.17752000 | −1.58513600 | −1.85706300 |
| H | 3.78771800 | −2.00383600 | −1.05269800 |
| H | 2.30291300 | −2.21831700 | −2.01017200 |
| H | 3.77168900 | −1.63153200 | −2.77365000 |

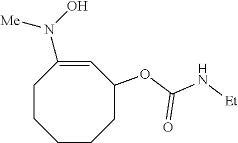 [SMILES: CCNC(=O)OC1/C=C(/N(C)O)CCCCC1]

| | | | |
|---|---|---|---|
| C | 1.36798100 | 3.05599000 | −0.00428300 |
| C | −0.07280900 | 2.59701900 | 0.23838200 |
| C | 2.35249200 | −0.44675900 | 0.21700400 |
| C | −0.24459000 | 1.22915200 | 0.90869300 |
| C | 1.08786600 | −0.83612300 | 0.04172200 |
| C | −0.12832900 | 0.04086400 | −0.07051900 |
| H | 1.81244100 | 3.38039400 | 0.94621600 |
| H | −0.62252900 | 2.56425000 | −0.70989200 |
| H | 0.44521000 | 1.09928500 | 1.74960300 |
| H | 1.33809800 | 3.94570900 | −0.64281900 |
| H | −0.57189800 | 3.35627100 | 0.84991200 |
| H | −1.25357000 | 1.18694200 | 1.32612500 |
| H | −0.22956100 | 0.42623500 | −1.09293500 |
| C | 2.30247300 | 2.01387200 | −0.63134300 |
| H | 3.15318400 | 2.54168800 | −1.07335900 |
| H | 1.80131400 | 1.50581300 | −1.46381300 |
| C | 2.84629600 | 0.97463700 | 0.37528100 |
| H | 2.62110200 | 1.30064600 | 1.39804400 |
| O | −1.24029600 | −0.86242600 | 0.12856400 |
| C | −2.42040000 | −0.42172100 | −0.35505900 |
| O | −2.58518200 | 0.65257000 | −0.90129800 |
| N | −3.39483900 | −1.34834300 | −0.16182500 |
| H | −3.16828600 | −2.13917800 | 0.42059500 |
| H | 3.93820100 | 0.95340600 | 0.33513400 |
| H | 0.89562200 | −1.89764300 | −0.06358700 |
| N | 3.42245800 | −1.39600200 | 0.35173200 |
| O | 2.92456700 | −2.71783200 | 0.47030800 |
| H | 2.97498900 | −2.88653300 | 1.41902000 |
| C | −4.77904900 | −1.01429800 | −0.44797200 |
| H | −5.33196400 | −1.95134700 | −0.55374000 |
| H | −4.79504900 | −0.50751400 | −1.41568200 |
| C | −5.40749800 | −0.12129000 | 0.61741500 |
| H | −4.84441400 | 0.81223700 | 0.68864800 |
| H | −6.44304200 | 0.11774600 | 0.36125100 |
| H | −5.39891200 | −0.61221200 | 1.59456300 |
| C | 4.28900300 | −1.43975700 | −0.82650400 |
| H | 5.13602300 | −2.09307400 | −0.61371000 |
| H | 3.73951600 | −1.82316500 | −1.69622600 |
| H | 4.65685800 | −0.43756200 | −1.05019100 |

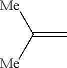

| | | | |
|---|---|---|---|
| C | −0.13448400 | 0.45888800 | 0.00002200 |
| H | −0.16905000 | 1.54766300 | −0.00008500 |
| C | −1.27570900 | −0.22235700 | −0.00004700 |
| H | −1.28354100 | −1.30928400 | 0.00000200 |
| H | −2.23849300 | 0.27733000 | 0.00019100 |
| C | 1.22995400 | −0.16280000 | −0.00000900 |
| H | 1.16566800 | −1.25359300 | −0.00065800 |
| H | 1.80368200 | 0.14831000 | −0.87915100 |
| H | 1.80316300 | 0.14718900 | 0.87990500 |

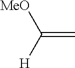

| | | | |
|---|---|---|---|
| C | −1.86585900 | −0.07072000 | 0.03506300 |
| H | −2.09454700 | −1.12824900 | 0.08720700 |
| H | −2.67711400 | 0.64420000 | 0.03213300 |
| C | −0.60863900 | 0.35157600 | −0.03320200 |
| H | −0.35113700 | 1.41028100 | −0.08480700 |
| O | 0.44398700 | −0.50497900 | −0.06097900 |
| C | 1.71042300 | 0.10968600 | 0.04099500 |
| H | 1.83239700 | 0.61751000 | 1.00530100 |
| H | 2.45496800 | −0.68185700 | −0.04186200 |
| H | 1.86798200 | 0.83469200 | −0.76728000 |

### Example 107: Synthesis of hydroxylamine-functionalized beads

Hydroxylamine-functionalized magnetic agarose beads

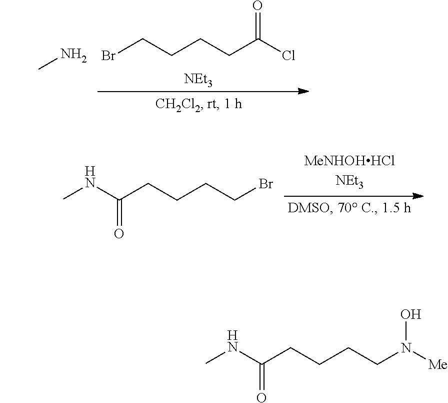

A 2 mL microcentrifuge tube was charged with a suspension of amine functionalized magnetic agarose beads in aqueous buffer (25% v/v, 1 mL, PureCube Amino MagBeads). The beads were washed with isopropanol (3×1 mL) and dichloromethane (3×1 mL) then resuspended in dichloromethane (1 mL). Triethylamine (13.9 μL, 100 μmol) and 5-bromovaleryl chloride (6.7 μL, 50.0 μmol) were sequentially added to the suspension at room temperature. The microcentrifuge tube was capped and the solution was rotated end over end. After 1 h, the beads were washed with dichloromethane (3×1 mL). N-methylhydroxylamine hydrochloride (20.9 mg, 250 μmol), dimethylsulfoxide (1.67 mL), and triethylamine (69.7 μL, 500 μmol) were then sequentially added to the beads. The tube was purged with nitrogen, capped, manually agitated, then heated to 70° C. After 1.5 h, the solution was allowed to cool to room temperature, and the beads were washed with methanol (3×1 mL). The beads were then resuspended in water (1 mL). The suspension was stored frozen at −20° C.

Bead loading was determined by charging a 1.6 mL microcentrifuge tube with the aqueous suspension of magnetic agarose beads (10 μL). A solution of Fmoc-Lys(cyclooct-2-yn-1-yloxycarbonyl)-OH (75 μL, 50 mM in 50% v/v methanol/dichloromethane) was added to the solution. After 30 min, the beads were washed with methanol (2×1 mL). A solution of piperidine in N,N-dimethylformamide (20% v/v, 1 mL) was then added to the beads. UV-vis absorbance (k=301 nm) of this solution was then measured, and the hydroxylamine loading was calculated.

Hydroxylamine-functionalized crosslinked agarose beads

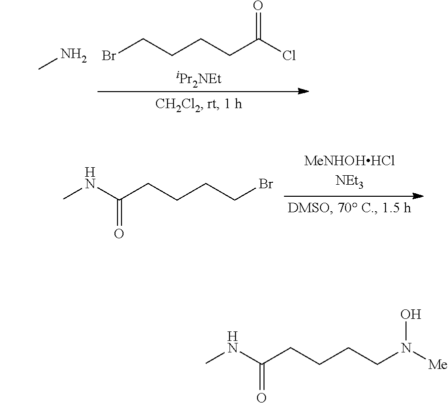

A fritted syringe was charged with a suspension of amino terminal crosslinked agarose gel with a 6-atom hydrophilic arm in aqueous buffer (15 μmol/mL, 20 mL, BioRad Affi-Gel 102). The beads were washed with methanol (3×10 mL) and isopropanol (3×10 mL). Separately, 5-bromovaleryl chloride (134 μL, 1.00 mmol) was added to a solution of N,N-diisopropylethylamine (209 μL, 1.20 mmol) in N,N-dimethylformamide (10 mL). This solution was added to the beads, and the syringe was plugged and rotated end over end at room temperature. After 1 h, the solution was drained, and the beads were washed with isopropanol (3×10 mL) and methanol (3×10 mL). The beads were then transferred to a vial and resuspended in dimethylsulfoxide (10 mL). Triethylamine (418 μL, 3.00 mmol) and N-methylhydroxylamine hydrochloride (125 mg, 1.50 mmol) were then sequentially added to the suspension. The vial was purged with nitrogen, capped, manually agitated, and then heated to 70° C. After 1.5 h, the solution was allowed to cool to room temperature, and the beads were washed with isopropanol (3×10 mL), methanol (3×10 mL), water (3×10 mL), and methanol (3×10 mL). The beads were then dried under reduced pressure and stored as a dry solid.

Bead loading was determined by charging a microcentrifuge spin filter with hydroxylamine-functionalized beads (11.2 mg). A solution of Fmoc-Lys(cyclooct-2-yn-1-yloxycarbonyl)-OH (4 mg, 7.71 μmol) in 50% v/v methanol/dichloromethane (200 μL) was added to the solution. After 30 min, the beads were drained and washed with methanol (5×500 μL) and spin dried in a microcentrifuge (10,000×g, 2 min). A solution of piperidine in N,N-dimethylformamide (20% v/v, 500 μL) was then added to the beads. After 20 min, the solution was collected by centrifugation (10,000×g, 30 s), and another volume of piperidine in N,N-dimethylformamide (20% v/v, 500 μL) was added to the beads then combined with the previous volume by centrifugation (10,000×g, 30 s). UV-vis absorbance (k=301 nm) of this solution was then measured, and the hydroxylamine loading was calculated.

Hydroxylamine-functionalized Tentagel beads

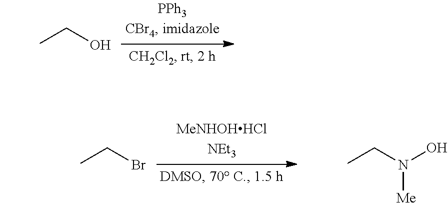

A vial was charged with Tentagel-S-OH beads (0.27 equiv/g, 250 mg, 67.5 μmol), triphenylphosphine (88.5 mg, 338 μmol), carbon tetrabromide (112 mg, 338 μmol), and imidazole (46.0 mg, 675 μmol). Dichloromethane (2 mL) was added, then the vial was capped and rotated end over end at room temperature. After 2 h, the beads were transferred to a filtered syringe and washed with dichloromethane (5×5 mL) and methanol (5×5 mL). The beads were dried under reduced pressure. The beads were then transferred to a vial, and N-methylhydroxylamine hydrochloride (28.2 mg, 338 μmol), dimethylsulfoxide (2 mL), and triethylamine (263 μL, 675 μmol) were sequentially added. The vial was purged with nitrogen, capped, manually agitated, then heated to 70° C. After 1.5 h, the solution was allowed to cool to room temperature, and the bead suspension was transferred to a syringe filter. The beads were washed with methanol (5×5 mL), dichloromethane (5×5 mL), and methanol (5×5 mL) then dried under reduced pressure.

Bead loading was determined by charging a microcentrifuge spin filter with hydroxylamine-functionalized beads (11.2 mg). A solution of Fmoc-Lys(cyclooct-2-yn-1-yloxycarbonyl)-OH (4 mg, 7.71 μmol) in 50% v/v methanol/dichloromethane (200 μL) was added to the solution. After 30 min, the beads were drained and washed with methanol (5×500 μL) and spin dried in a microcentrifuge (10,000×g, 2 min). A solution of piperidine in N,N-dimethylformamide (20% v/v, 500 μL) was then added to the beads. After 20 min, the solution was collected by centrifugation (10,000×g, 30 s), and another volume of piperidine in N,N-dimethylformamide (20% v/v, 500 μL) was added to the beads then combined with the previous volume by centrifugation (10,000×g, 30 s). UV-vis absorbance (k=301 nm) of this solution was then measured, and the hydroxylamine loading was calculated.

Synthesis of Fmoc-Lys(cyclooct-2-yn-1-yloxycarbonyl)-OH

Triethylamine (36.1 μL, 207 μmol) was added via syringe to a solution of Fmoc-Lys-OH (50.9 mg, 138 μmol) and cyclooct-2-yn-1-yl (4-nitrophenyl) carbonate (20.0 mg, 69.1 μmol) in N,N-dimethylformamide (1.5 mL) at room temperature. After 4 h, the solution was diluted with water and directly purified by automated C₁₈ reverse phase column chromatography (30 g C₁₈ silica gel, 25 μm spherical particles, eluent: H₂O+0.1% TFA (5 CV), gradient 0→100% MeCN/H₂O+0.1% TFA (10 CV)).

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A compound having a structure represented by formula I: 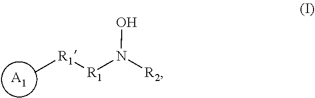
or a pharmaceutically acceptable salt or stereoisomer thereof,
wherein:
R₁′ is a linking group;
R₁ is absent, or
R₁ and R₂, together with the nitrogen atom to which they are attached, form a heterocyclyl;
R₂ is optionally substituted (C₁-C₈) alkyl, —C(O)R″, —C(O)OR″, —C(O)NR″R″, —S(O)R″, —S(O)₂R″, (C₃-C₁₀) carbocyclyl, 4- or 7-membered heterocyclyl, or a substituted polyethylene glycol chain, wherein each R″ is independently hydrogen, (C₁-C₆) alkyl, (C₃-C₁₀) carbocyclyl, 4- or 7-membered heterocyclyl, and wherein said alkyl, carbocyclyl, or heterocyclyl is optionally substituted; and
A₁ is an active moiety.

2.-31. (canceled)

32. A compound having a structure represented by formula II or III: 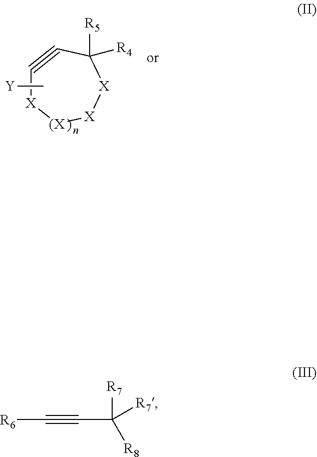
or a pharmaceutically acceptable salt or stereoisomer thereof,
wherein:
each X is independently CR₉R₉′, NR₉, O, S, C(O), S(O), or SO₂, wherein the ring system contains 0-3 heteroatoms;
R₉ and R₉′ are independently hydrogen or a substituent;
Y is absent or 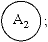
A₂ is an active moiety;
R₄ is hydrogen, a substituent or a linking group bound to an 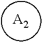
group, or
R₄ and R₅, together with the carbon atom to which they are attached, form a carbocyclyl or a heterocyclyl, wherein R₄ is also bound to an 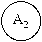
group;
R₅ is hydrogen or an electron withdrawing group;
R₆ is hydrogen, a π-electron donor group, or a linking group bound to an 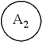
group;
R₇ and R₇′ are independently hydrogen or an electron withdrawing group, or
R₇ and R₇′, together with the carbon atom to which they are attached, form C(O);
R₈ is hydrogen, a substituent, or a linking group bound to an 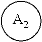
group; and
n is 1, 2, or 3,
provided that the compound contains an 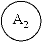
group.

33.-89. (canceled)

90. A compound having a structure represented by formula IV or V: 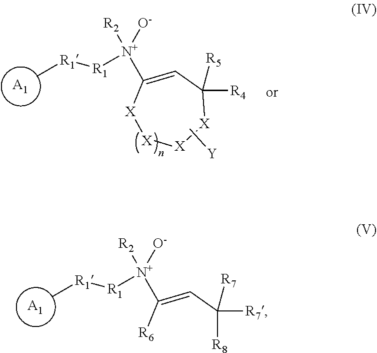
or a pharmaceutically acceptable salt or stereoisomer thereof,
wherein:
R₁′ is a linking group;
R₁ is absent, or
R₁ and R₂, together with the nitrogen atom to which they are attached, form a heterocyclyl;
R₂ is optionally substituted (C₁-C₈) alkyl, —C(O)R″, —C(O)OR″, —C(O)NR″R″, —S(O)R″, —S(O)₂R″, (C₃-C₁₀) carbocyclyl, 4- or 7-membered heterocyclyl, or a substituted polyethylene glycol chain, wherein each R″ is independently hydrogen, (C₁-C₆) alkyl, (C₃-C₁₀) carbocyclyl, 4- or 7-membered heterocyclyl, and wherein said alkyl, carbocyclyl, or heterocyclyl is optionally substituted;
each X is independently CR₉R₉′, NR₉, O, S, C(O), S(O), or SO₂, wherein the ring system contains 0-3 heteroatoms;
R₉ and R₉′ are independently hydrogen or a substituent;
A₁ is an active moiety;
Y is absent or 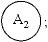
A₂ is an active moiety;
R₄ is hydrogen, a substituent or a linking group bound to 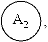
or
R₄ and R₅, together with the carbon atom to which they are attached, form a carbocyclyl or a heterocyclyl, wherein R₄ is also bound to 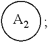
R₅ is hydrogen or an electron withdrawing group;
R₆ is hydrogen, a π-electron donor group, or a linking group bound to 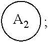
R₇ and R₇′ are independently hydrogen or an electron withdrawing group, or
R₇ and R₇′, together with the carbon atom to which they are attached, form C(O);
R₈ is hydrogen, a substituent, or a linking group bound to 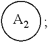
and
n is 1, 2, or 3;
provided that the compound contains at least one 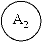
group.

91. The compound of claim 90, wherein R₁ is an alkylene chain, which may be interrupted by, and/or terminate (at either or both termini) in at least one of —O—, —O—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different, or
R₁ is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁2 carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different, or
wherein R₁ and R₂, together with the nitrogen atom to which they are attached, form a 5- to −10-membered heterocyclyl containing 1-3 heteroatoms selected from N, O, and S, or wherein R₂ is methyl, ethyl, isopropyl, or t-butyl.

92. The compound of claim 91, wherein the alkylene chain is a C₁-C₁₂ alkylene chain, or
wherein the polyethylene glycol chain has 1 to 20 —(CH₂CH₂—O—)— units.

93.-96. (canceled)

97. The compound of claim 90, wherein A₁ is a binding moiety, a therapeutic moiety, or a diagnostic moiety.

98. The compound of claim 97, wherein the binding moiety is a small molecule, a short amino acid sequence, a protein, or an antibody or a fragment thereof that binds a predetermined target, or
wherein the therapeutic moiety is a non-targeted cancer agent, a targeted anti-cancer agent, an anti-bacterial agent, a non-steroidal anti-inflammatory drug (NSAID), a corticosteroid, or a disease-modifying antirheumatic drug (DMARD), or
wherein the diagnostic moiety is a fluorophore, a chromogenic agent, a positron emission tomography (PET) tracer, or a magnetic resonance imaging (MRI) contrast agent.

99. The compound of claim 98, wherein the antibody is a monoclonal antibody or a binding fragment thereof, or
wherein the small molecule is biotin or a derivative thereof, a small molecule that binds an E3 ligase, or a small molecule that binds a cellular protein, or
wherein the targeted anti-cancer agent is a kinase inhibitor.

100.-108. (canceled)

109. The compound of claim 90, wherein n is 2, or
wherein X is CR₉R₉′, or
wherein R₄ is a linking group bound to 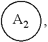
or
wherein R₄ and R₅, together with the carbon atom to which they are attached, form a 5- to −10-membered carbocyclyl or 5- to 10-membered heterocyclyl containing 1-3 heteroatoms selected from N, O, and S, or
R₅ is hydrogen, an inductive electron withdrawing group, or a π-electron withdrawing group.

110. (canceled)

111. The compound of claim 109, wherein R₉ and R₉′ are each hydrogen, or
wherein R₄ is O, S, NR₁₁, OPh, OC(O), or OC(O)NR₁₁, wherein R₁₁ is hydrogen or (C₁-C₆) alkyl, or
R₄ is an alkylene chain, which may be interrupted by, and/or terminate (at either or both termini) in at least one of —O—, —O—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different, or
R₄ is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁2 carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different, or
wherein R₄ and R₅, together with the carbon atom to which they are attached, form a 5-membered heterocyclyl containing 2-oxygen atoms, or
R₅ is an inductive electron withdrawing group, wherein the inductive electron withdrawing group is halogen, OR_{5′}, SR_{5′}, or NR_{5′}R_{5′}, wherein each R_{5′} is independently hydrogen, C₁-C₆ alkyl, C₆-C₁₂ aryl, 5- to 10-membered heteroaryl, carbonyl, sulfonyl, sulfinyl, or phosphoryl, or
R₅ is a π-electron withdrawing group, wherein the π-electron withdrawing group is —C(O)R_{5″}, —C(O)NR_{5″}R_{5″}, —C(O)NR_{5″}R_{5″}, —C(O)OR_{5″}, NO₂, CN, N₃, —S(O)R_{5″}, —S(O)₂R₅″, —S(O)OR_{5″}, —S(O)₂OR_{5″}, —S(O)NR_{5″}R_{5″}, —S(O)₂NR_{5″}R_{5″}, —OP(O)OR_{5″}OR_{5″}, —P(O)NR_{5″}R_{5″}NR_{5″}R_{5″}, wherein each R₅″ is independently hydrogen, C₁-C₆ alkyl, C₆-C₁₂ aryl, 5- to 10-membered heteroaryl.

112.-120. (canceled)

121. The compound of claim 111, wherein the alkylene chain is a C₁-C₁₂ alkylene chain, or
wherein the polyethylene glycol chain has 1 to 20 —(CH₂CH₂—O)— units.

122.-131. (canceled)

132. The compound claim 90, wherein R₆ is hydrogen or a π-electron donor group, or
wherein R₇ and R₇′ are independently hydrogen or an inductive electron withdrawing group, or
R₇ and R₇′ are independently hydrogen or a π-electron withdrawing group.

133. (canceled)

134. The compound of claim 132, wherein R₆ is OR₁₂, SR₁₂, NR₁₂NR₁₂, or a cyclic or acyclic amide, wherein each R₁₂ is independently hydrogen, (C₁-C₆) alkyl, (C₃-C₁₀) carbocyclyl, 4- or 7-membered heterocyclyl, wherein said alkyl, carbocyclyl, or heterocyclyl is optionally substituted, or
wherein the inductive electron withdrawing group is halogen, OR_{5′}, SR_{5′}, or NR₅′R_{5′}, wherein each R_{5′} is independently hydrogen, C₁-C₆ alkyl, C₆-C₁2 aryl, 5- to 10-membered heteroaryl, carbonyl, sulfonyl, sulfinyl, or phosphoryl, or
wherein the π-electron withdrawing group is —C(O)R_{5″}, —C(O)NR_{5″}R_{5″}, —C(O)NR_{5″}R_{5″}, —C(O)OR_{5″}, NO₂, CN, N₃, —S(O)R_{5″}, —S(O)₂R_{5″}, —S(O)OR_{5″}, —S(O)₂OR_{5″}, —S(O)NR_{5″}R_{5″}, —S(O)₂NR_{5″}R_{5″}, —OP(O)OR_{5″}OR_{5″}, —P(O)NR_{5″}R_{5″}NR_{5″}R_{5″}, wherein each R_{5″} is independently hydrogen, C₁-C₆ alkyl, C₆-C₁₂ aryl, 5- to 10-membered heteroaryl.

135.-138. (canceled)

139. The compound of claim 90, wherein R₈ is a linking group bound to

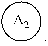

140. The compound of claim 139, wherein R₈ is CH₂, aryl, or O, or R₈ is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁2 carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different, or
R₈ is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

141.-143. (canceled)

144. The compound of claim 140, wherein the alkylene chain is a C₁-C₁₂ alkylene chain or wherein the polyethylene glycol chain has 1 to 10 —(CH₂CH₂—O)— units.

145. (canceled)

146. (canceled)

147. The compound of claim 90, wherein A₂ is a binding moiety, a therapeutic moiety, or a diagnostic moiety.

148. The compound of claim 147, wherein the binding moiety is a small molecule, a short amino acid sequence, a protein, or an antibody or a fragment thereof that binds a predetermined target, or
wherein the therapeutic moiety is a non-targeted cancer agent, a targeted anti-cancer agent, an anti-bacterial agent, a non-steroidal anti-inflammatory drug (NSAID), a corticosteroid, or a disease-modifying antirheumatic drug (DMARD), or
wherein the diagnostic moiety is a fluorophore, a chromogenic agent, a positron emission tomography (PET) tracer, or a magnetic resonance imaging (MRI) contrast agent.

149. The compound of claim 148, wherein the antibody is a monoclonal antibody or a binding fragment thereof, or
wherein the small molecule is biotin or a derivative thereof, a small molecule that binds an E3 ligase, or a small molecule that binds a cellular protein, or
wherein the targeted anti-cancer agent is a kinase inhibitor.

150.-158. (canceled)

159. The compound of claim 90, which is 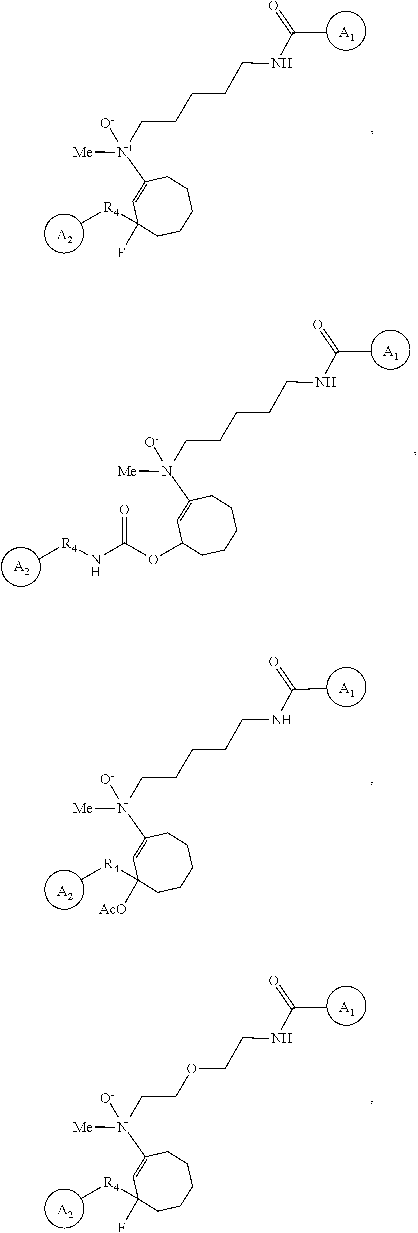 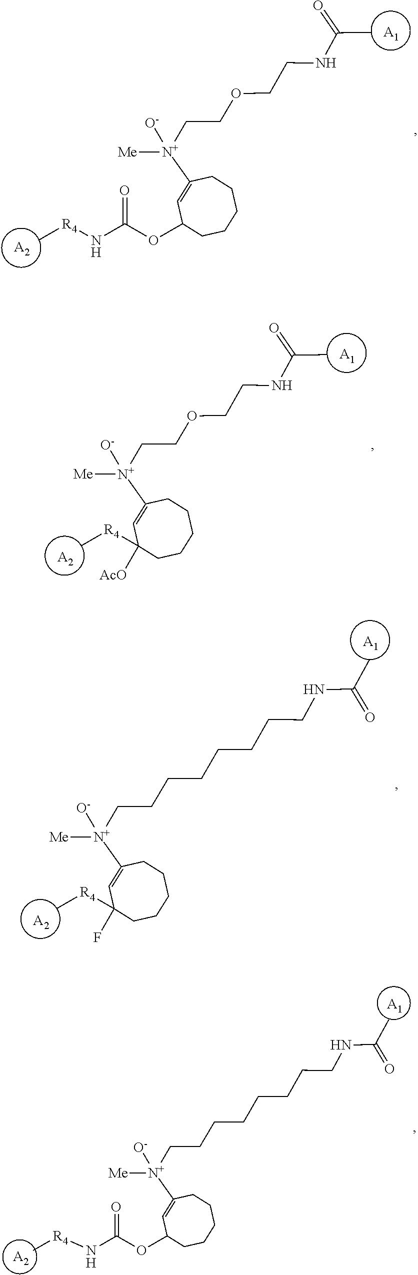 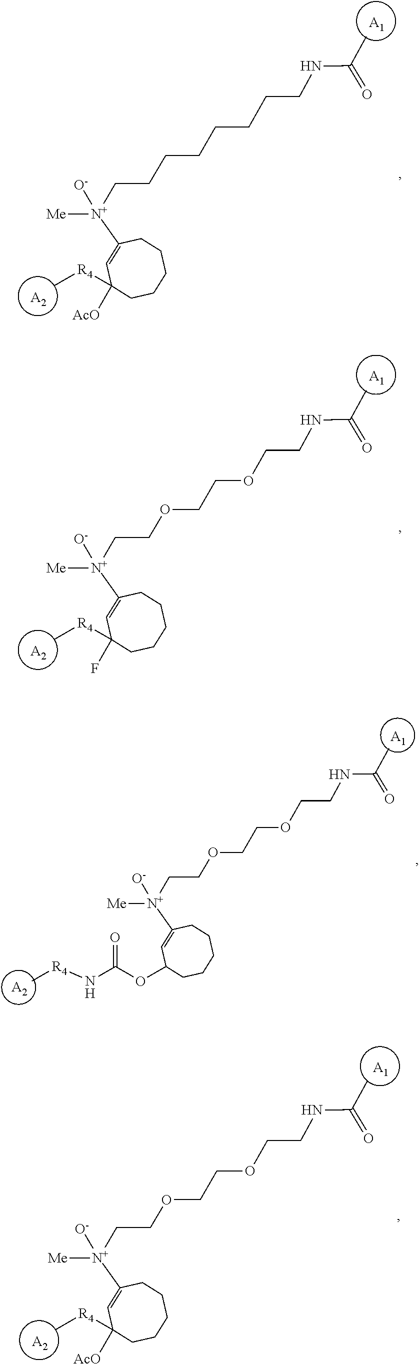 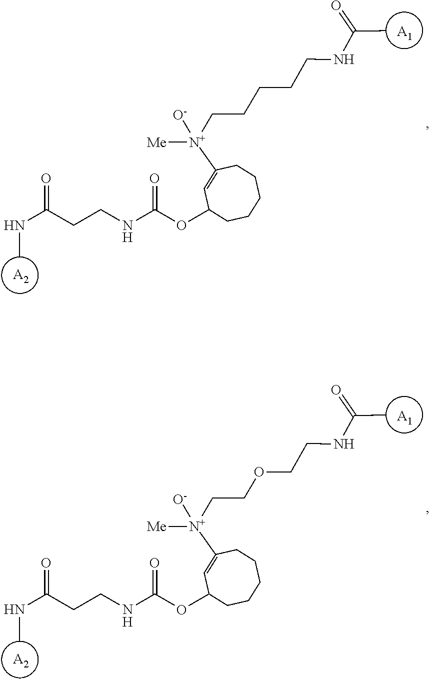
or a pharmaceutically acceptable salt or stereoisomer thereof, or 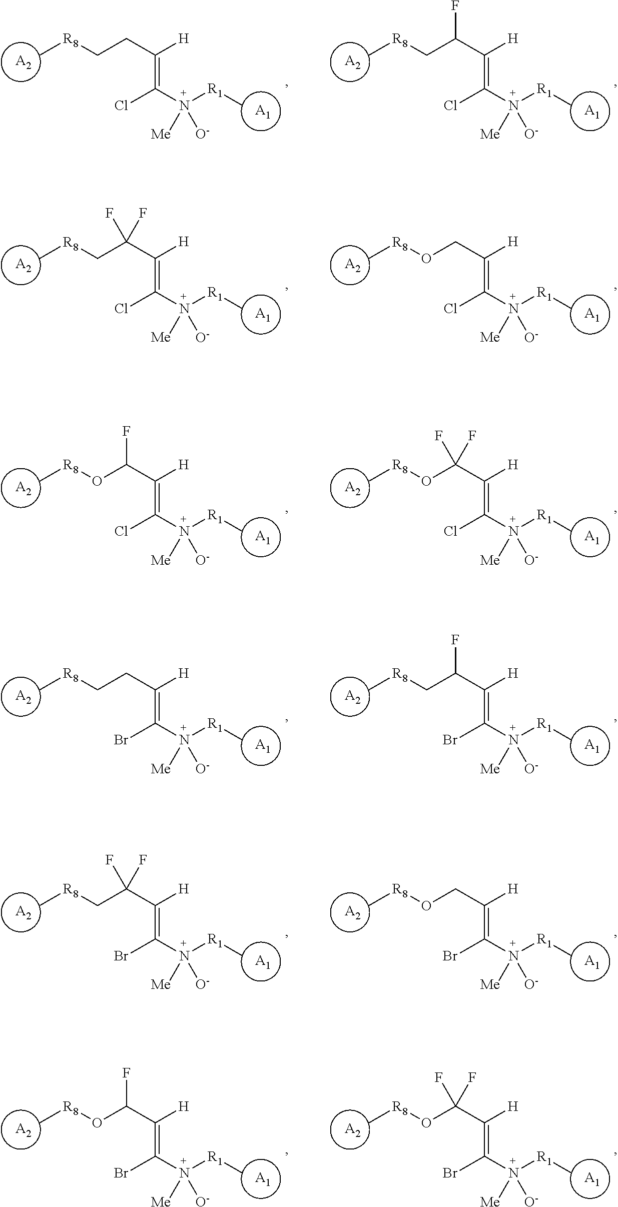 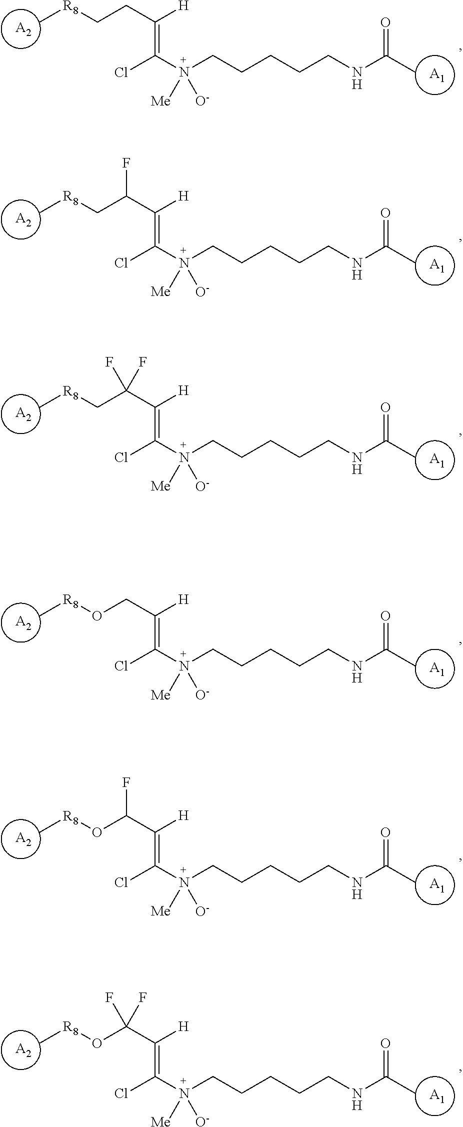 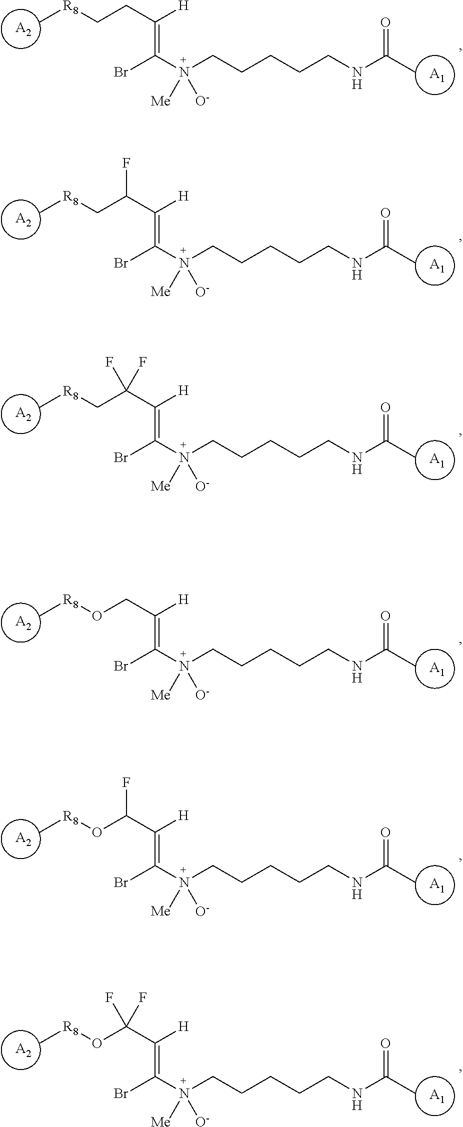 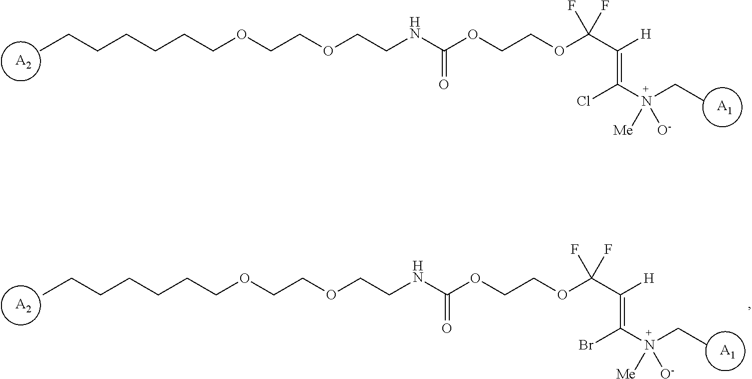
or a pharmaceutically acceptable salt or stereoisomer thereof.

160. The compound of claim 159, which is 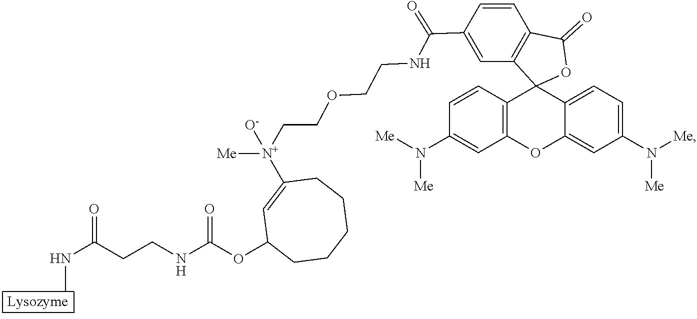 [SMILES: CNC(=O)CCNC(=O)OC1/C=C(/[N+](C)([O-])CCOCCNC(=O)c2ccc3c(c2)C2(OC3=O)c3ccc(N(C)C)cc3Oc3cc(N(C)C)ccc32)CCCCC1]
or a pharmaceutically acceptable salt or stereoisomer thereof, or 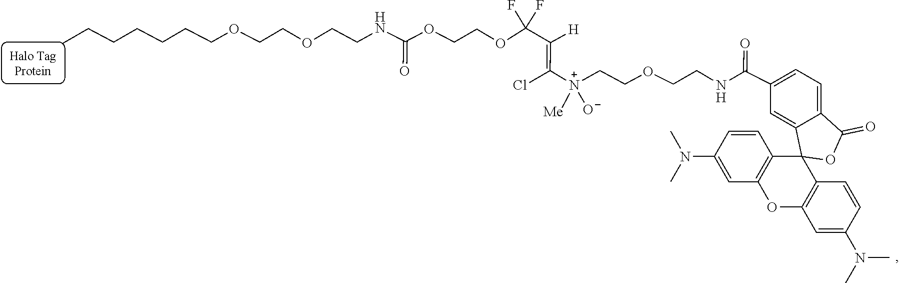 [SMILES: CCCCCCCOCCOCCNC(=O)OCCOC(F)(F)/C=C(\Cl)[N+](C)(O)CCOCCNC(=O)c1ccc2c(c1)C1(OC2=O)c2ccc(N(C)C)cc2Oc2cc(N(C)C)ccc21]
or a pharmaceutically acceptable salt or stereoisomer thereof.

161. (canceled)

162. (canceled)

163. The compound of claim 90, wherein one of 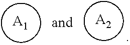
and is a diagnostic agent and the other is a therapeutic agent, and the compound is in the form of a theranostic agent, or
wherein one of 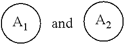
is an antibody or binding fragment thereof and the other is a therapeutic agent, and the compound is in the form of an antibody-drug conjugate, or
wherein both of 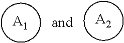
and are binding agents, and the compound is a degrader.

164. (canceled)

165. (canceled)

166. The compound of claim 90, wherein the compound of formula IV is of formula IVa′, IVb′, or IVc′: 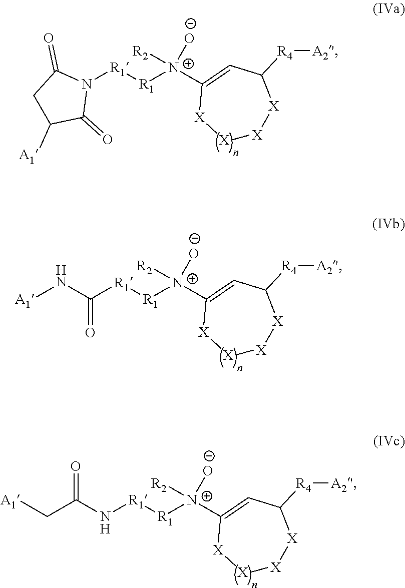
or pharmaceutically acceptable salt or stereoisomer thereof.

167. The compound of claim 166, wherein, the antibody is a monoclonal antibody, R₁ and R₂, together with the nitrogen atom to which they are attached, form a piperazinyl, and has a structure represented by formula IVa′1: 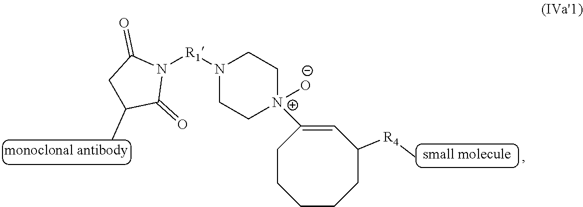
or a pharmaceutically acceptable salt or stereoisomer thereof, or
wherein the antibody is a monoclonal antibody, R₁ is absent and R₂ is methyl, and has a structure represented by formula IVa′2: 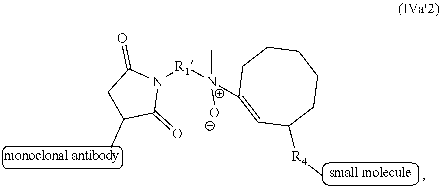
or a pharmaceutically acceptable salt or stereoisomer thereof.

168. (canceled)

169. A pharmaceutical composition, comprising a therapeutically effective amount of the compound or pharmaceutically acceptable salt or stereoisomer of claim 90, and a pharmaceutically acceptable carrier.

170. A method of treating cancer, comprising administering to a subject in need thereof a therapeutically effective amounts of the compound or pharmaceutically acceptable salt or stereoisomer of claim 166 and a diboron reagent.

171. (canceled)

172. (canceled)

173. A method of treating a disease or disorder, comprising administering to a subject in need thereof a therapeutically effective amount of the compound or pharmaceutically acceptable salt or stereoisomer of claim 90, which is therapeutic.

174.-176. (canceled)

177. A method of protein labeling, comprising administering the compound or pharmaceutically acceptable salt or stereoisomer of claim 90, wherein one active moiety binds a protein and the other active moiety is a diagnostic moiety (label).

178. (canceled)

179. A process of preparing a compound of formula IV: 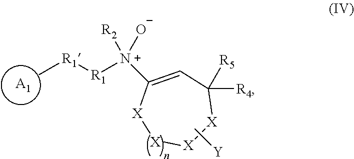
comprising reacting a compound of formula I: 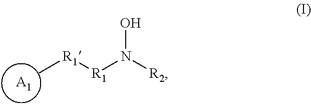
with a compound of formula II: 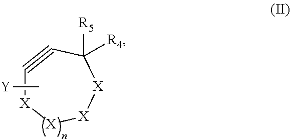
or
a process of preparing a compound of formula V: 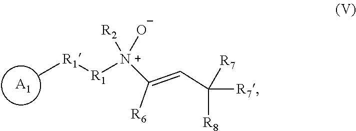
comprising reacting a compound of formula I: 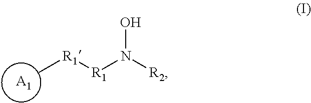
with a compound of formula III:

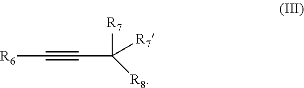

180.-202. (canceled)


SEQUENCE LISTING (7 sequences)

SEQ ID NO: 1 - DNA, 975 bp, Artificial Sequence
  Synthetic Polynucleotide
aataattttgtttaactttaagaaggagatataccctcgagatgggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggtgagcaccaccaccaccaccactgagatccggctgctaaca

SEQ ID NO: 2 - DNA, 1190 bp, Artificial Sequence
  SignalSeq-HaloTag-PDGFR Gene
actatagggctagcgccaccatggagacagacacactcctgctatgggtactgctgctctgggttccaggttccactggtgactatccatatgatgttccagattatgctggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggtgaaaacctgtacttccaatccgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtggtggttctggtatggttagc

SEQ ID NO: 3 - DNA, 726 bp, Artificial Sequence
  sfGFP Gene
atggttagcaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccatgcggtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagctaa

SEQ ID NO: 4 - DNA, 19 bp, Artificial Sequence
  SignalSeq-HaloTag-PDGFR-Gibson-F
actatagggctagcgccac

SEQ ID NO: 5 - DNA, 21 bp, Artificial Sequence
  SignalSeq-HaloTag-PDGFR-Gibson-R
gctaaccataccagaaccacc

SEQ ID NO: 6 - DNA, 26 bp, Artificial Sequence
  pHTC-sfGFP-Gibson-F
ggtggttctggtatggttagcaaagg

SEQ ID NO: 7 - DNA, 21 bp, Artificial Sequence
  pHTC-sfGFP-Gibson-R
ggtggcgctagccctatagtg